(19) United States
(12) Patent Application Publication
(10) Pub. No.: US 2016/0046616 A1
(43) Pub. Date: Feb. 18, 2016

(54) NRF2 SMALL MOLECULE INHIBITORS FOR CANCER THERAPY

(71) Applicants: THE JOHNS HOPKINS UNIVERSITY, Baltimore, MD (US); The National Institutes of Health, Bethesda, MD (US)

(72) Inventors: SHYAM S. BISWAL, BALTIMORE, MD (US); ANJU SINGH, BALTIMORE, MD (US); FRAYDOON RASTINEJAD, ORLANDO, FL (US); MIN SHEN, ROCKVILLE, MD (US); MATTHEW B. BOXER, ROCKVILLE, MD (US); YA-QIN ZHANG, ROCKVILLE, MD (US); JASON M. ROHDE, ROCKVILLE, MD (US); KYU OH, BALTIMORE, MD (US); SREEDHAR VENKANNAGARI, BALTIMORE, MD (US)

(73) Assignees: THE JOHNS HOPKINS UNIVERSITY, BALTIMORE, MD (US); THE NATIONAL INSTITUTES OF HEALTH, BETHESDA, MD (US); SANFORD BURNHAM PREBYS MEDICAL DISCOVERY INSTITUTE, La Jolla, CA (US)

(21) Appl. No.: 14/774,956

(22) PCT Filed: Mar. 17, 2014
(86) PCT No.: PCT/US14/30442

§ 371 Date: Sep. 11, 2015

Related U.S. Application Data
(60) Provisional application No. 61/798,843, filed on Mar. 15, 2013.

(51) Int. Cl. C07D 417/12; C07D 277/46; C07D 307/68; C07D 405/12; C07D 417/04; C07D 471/08; C07D 401/04; C07D 213/75; C07D 471/04; C07D 403/04; C07D 495/04; C07D 217/04; C07D 417/14; C07D 405/14

(52) U.S. Cl. CPC C07D 417/12; C07D 417/14; C07D 277/46; C07D 307/68; C07D 405/12; C07D 417/04; C07D 405/14; C07D 401/04; C07D 213/75; C07D 471/04; C07D 403/04; C07D 495/04; C07D 217/04; C07D 471/08

(57) ABSTRACT

Small molecule inhibitors of Nrf2 and methods of their use are provided for treating or preventing a disease, disorder or condition associated with an Nrf2-regulated pathway. The compound can be administered as a single agent or can be administered to enhance the efficacy of a chemotherapeutic drug and/or radiation therapy.

### CROSS-REFERENCE TO RELATED APPLICATION

This application claims the benefit of U.S. Provisional Application No. 61/798,843, filed Mar. 15, 2013, which is incorporated herein by reference in its entirety.

### FEDERALLY SPONSORED RESEARCH OR DEVELOPMENT

This invention was made with government support under R01CA140492, P50CA058184, and R03MH092170, awarded by the National Institutes of Health (NIH). The government has certain rights in the invention.

### BACKGROUND

Resistance to chemotherapy and radiotherapy remains a major obstacle in the successful treatment of cancer. Resistance may occur during cancer treatment because of many reasons, such as some of the cancer cells that are not killed can mutate and become resistant, gene amplification resulting in the overexpression of a protein that renders the treatment ineffective may occur, or cancer cells may develop a mechanism to inactivate the treatment.

Nuclear factor erythroid-2 related factor-2 (Nrf2) is a redox-sensitive transcription factor that regulates the expression of electrophile and xenobiotic detoxification enzymes and efflux proteins, which confer cytoprotection against oxidative stress and apoptosis in normal cells.

Cancer cells show greater expression of drug detoxification enzymes and efflux pumps. This characteristic can result in cancer therapeutic resistance due to the ability of a cancer cell to eliminate a toxic drug, such as a chemotherapeutic drug, from the cell. Further, a gain of Nrf2 in cancer can cause an increased expression of drug detoxification enzymes and efflux pumps. Without wishing to be bound to any one particular theory, is thought that the gain of Nrf2 occurs in various cancers is due to activating mutation in Nrf2 or mutation in the inhibitor kelch-like ECH-associated protein 1 (Keap1), as well as activation by many mechanisms as a result of activation of several oncogenes.

### SUMMARY

The presently disclosed subject matter provides compositions and methods that improve the efficacy of chemotherapy and radiotherapy leading to improved overall survival of a subject afflicted with cancer. Specifically, compositions and methods involving Nrf2 inhibitors are provided that can be used as single therapeutic agents or in combination with conventional chemotherapeutic drugs or along with ionizing radiation to make cancer cells less resistant to chemotherapy and/or radiation treatment.

Accordingly, in one aspect, the presently disclosed subject matter provides compound selected from the group consisting:

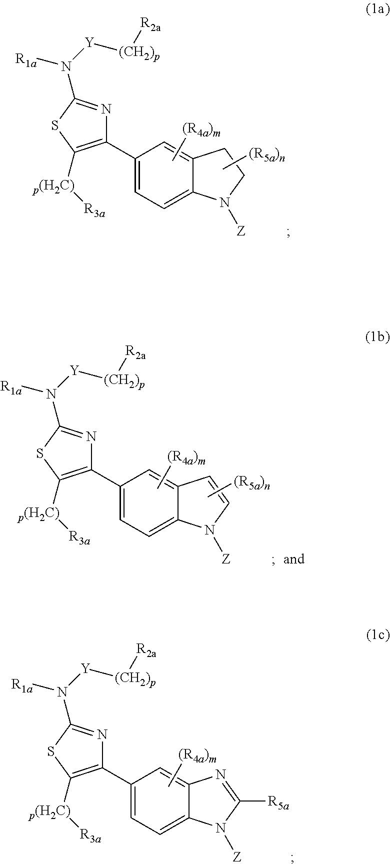

wherein:

m is an integer selected from the group consisting of 0, 1, 2, and 3;

n is an integer selected from the group consisting of 0, 1, and 2;

each p is independently an integer selected from the group consisting of 0, 1, and 2;

R₁ₐ is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R₂ₐ is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

R₃ₐ is selected from the group consisting of H and substituted or unsubstituted straight-chain or branched alkyl;

each R₄ₐ and R₅ₐ is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R₆ₐ is selected from the group consisting of H, substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

Y is —(C═O)— or —S(═O)₂—; and

Z is selected from the group consisting of R₆ₐ, —C(═O)—(CH₂)ₚ—R₆ₐ, and —S(═O)₂—R₆ₐ; p is an integer selected from the group consisting of 0, 1, and 2;

or an enantiomer, diastereomer, racemate or pharmaceutically acceptable salt, prodrug, or solvate thereof;
under the proviso that the compound of formula (1a) is not a compound selected from the group consisting of:

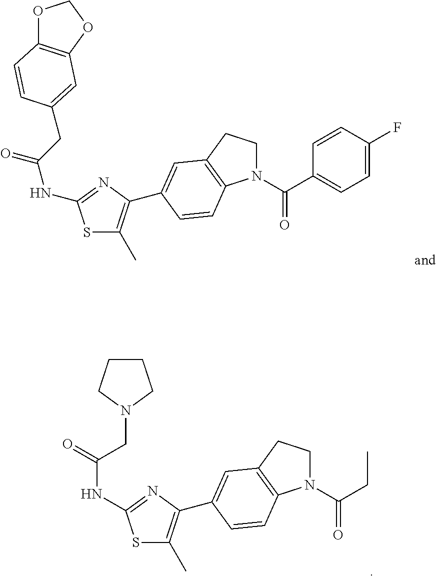 [SMILES: Cc1sc(NC(=O)Cc2ccc3c(c2)OCO3)nc1-c1ccc2c(c1)CCN2C(=O)c1ccc(F)cc1 ; CCC(=O)N1CCc2cc(-c3nc(NC(=O)CN4CCCC4)sc3C)ccc21]

In yet other aspects, the presently disclosed subject matter provides a compound selected from the group consisting of:

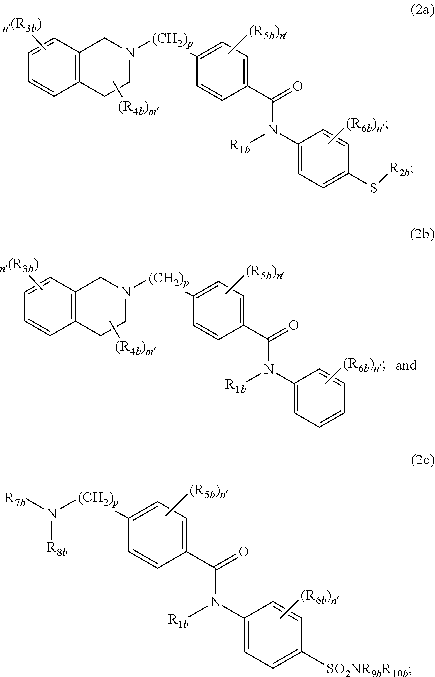

wherein:

m′ is an integer selected from the group consisting of 0, 1, 2, and 3;

n′ is an integer selected from the group consisting of 0, 1, 2, 3, and 4;

each p is independently an integer selected from the group consisting of 0, 1, and 2;

R_{1b} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R_{2b} is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

each R_{3b}, R_{4b}, R_{5b}, or R_{b6} is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

or for compounds of formula (2b) at least one R_{6b} is selected from the group consisting of H, substituted or unsubstituted cycloheteroalkyl, —(CH₂)ₚ—R_{6b}, and —C(═O)—R_{6b}; p is an integer selected from the group consisting of 0, 1, and 2;

R_{7b} and R_{8b} are each independently selected from the group consisting of substituted or unsubstituted straight-chain or branched alkyl and substituted or unsubstituted cycloheteroalkyl, or R_{7b} and R_{8b} can together form a substituted or unsubstituted heterocyclic ring;

R_{9b} and R_{10b} are each independently selected from the group consisting of substituted or unsubstituted straight-chain or branched alkyl and substituted or unsubstituted cycloheteroalkyl, or R_{9b} and R_{10b} can together form a substituted or unsubstituted heterocyclic ring;

or an enantiomer, diastereomer, racemate or pharmaceutically acceptable salt, prodrug, or solvate thereof;

under the proviso that if the compound is a compound of formula (2a), R_{2b} cannot be —CH₃ or —(O)₂OH.

In yet other aspects, the presently disclosed subject matter provides a compound of formula (3):

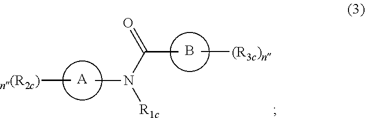

wherein:

each n″ is an integer independently selected from the group consisting of 0, 1, 2, 3, 4, 5, and 6, depending on the maximum available atoms on ring A and ring B;

A is a ring structure selected from the group consisting of:

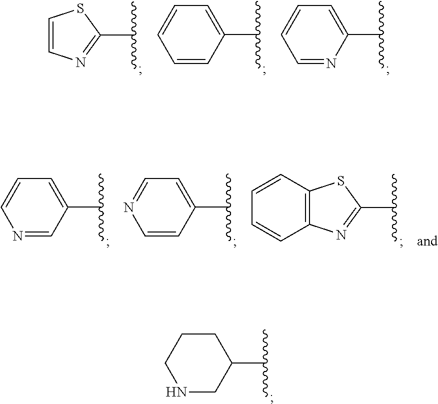

and

B is —(CH₂)ₙ— or a ring structure selected from the group consisting of:

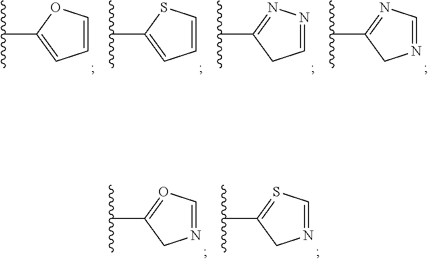

wherein the ring structure A and ring structure B are connected via an amide linkage represented by —NR_{1c}C(═O)—;

R_{1c} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R_{2c} and R_{3c} are each independently selected from the group consisting of substituted or unsubstituted straight-chain or branched alkyl and —(CH₂)ₚ—Cy,

wherein p is an integer selected from the group consisting of 0, 1, and 2; and Cy is selected from the group consisting of substituted or unsubstituted cycloheteroalkyl, substituted or unsubstituted aryl, and substituted or unsubstituted heteroaryl;

or an enantiomer, diastereomer, racemate or pharmaceutically acceptable salt, prodrug, or solvate thereof.

In other aspects, the presently disclosed subject matter provides a method for treating or preventing a disease, disorder or condition associated with an Nrf2-regulated pathway, the method comprising administering at least one presently disclosed compound of formula (1), formula (2), or formula (3) to a subject in an amount effective to decrease Nrf2 expression, thereby treating or preventing the disease, disorder, or condition.

In another aspect, the presently disclosed subject matter provides a method for treating or preventing a disease, disorder or condition associated with an Nrf2-regulated pathway, the method comprising administering at least one presently disclosed compound of formula (1), formula (2), or formula (3) to a subject in an amount effective to decrease Nrf2 expression, and wherein the compound is administered before, during, or after administration of a chemotherapeutic drug and/or a radiation therapy to the subject.

In still another aspect, the presently disclosed subject matter provides a method for downregulating at least one chemoresistant gene or radioresistant gene, the method comprising administering at least one presently disclosed compound of formula (1), formula (2), or formula (3) to a subject in an amount effective to downregulate at least one chemoresistant gene or radioresistant gene.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows a representative screening strategy for identifying small molecule inhibitors of Nrf2;

FIG. 2 shows the structure of compound 1, as well as results from real time PCR based validation assays, fluorescence polarization assays, and clonogenic assays;

FIG. 3 shows the structure of compound 4, as well as results from real time PCR based validation assays, fluorescence polarization assays, and clonogenic assays;

FIG. 4 shows the structure of compound 3, as well as results from real time PCR based validation assays and fluorescence polarization assays;

FIG. 5 shows the structure, real time PCR based validation assays, and pharmacokinetic plasma profile in CD1 mice of compound 3;

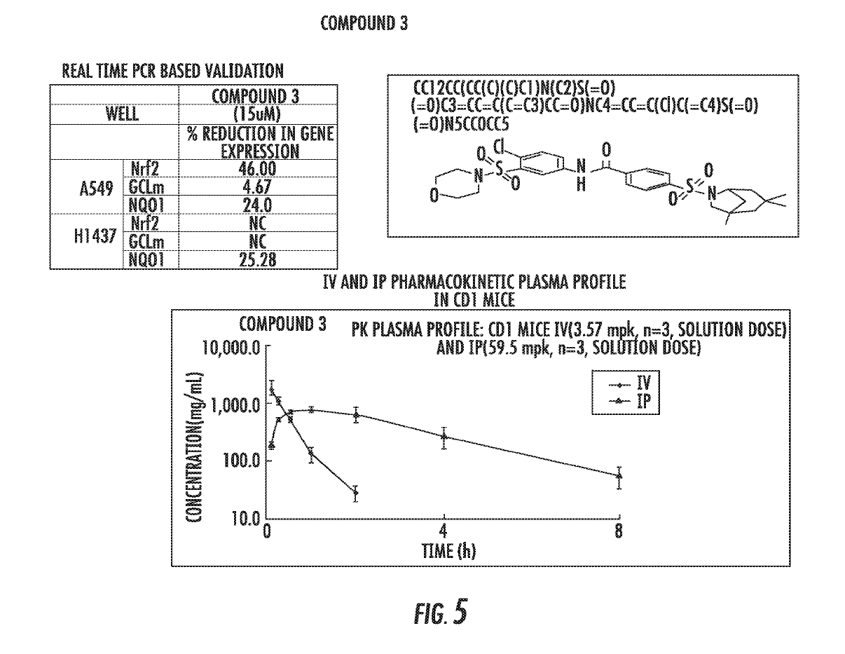

FIG. 6 shows the effect of compound 3 in combination with chemotherapeutic drugs etoposide, cisplatin, and carboplatin, in A549 lung cancer cells;

FIG. 7 shows the effect of compound 3 in combination with chemotherapeutic drugs etoposide, cisplatin, and carboplatin, in H460 lung cancer cells;

FIG. 8 shows the effect of compound 3 on the growth of A549 and H460 xenograft tumors in vivo alone or in combination with the chemotherapeutic drug carboplatin;

FIG. 9 shows the structure, real time PCR based validation assays, and clonogenic assays of compound 4;

FIG. 10 shows the effect of compound 4 on the cytotoxicity of the chemotherapeutic drug paclitaxel in H460 lung cancer cells;

FIG. 11 shows the pharmacokinetic plasma profile of compound 4 in CD1 mice, as well as its effect on the growth of A549 xenograft tumors in vivo as a single agent and in combination with the chemotherapeutic drug carboplatin;

FIG. 12 shows the structure, real time PCR based validation assays, and clonogenic assays of compound 1;

FIG. 13 shows the pharmacokinetic plasma profile of compound 1 in CD1 mice, as well as its effect on the growth of A549 xenograft tumors in vivo as a single agent and in combination with the chemotherapeutic drug carboplatin;

FIG. 14 shows the suppression of growth of rhabdomyosarcoma cells by compounds 3, 4, and 1 as single agents in a clonogenic assay;

FIG. 15 shows the suppression of growth of osteosarcoma cells by compounds 3, 4, and 1 as single agents and in combination with the chemotherapeutic drug doxorubicin in a clonogenic assay;

FIG. 16 shows the suppression of growth of pancreatic cancer cells (Panc1) by compounds 3, 4, and 1 as single agents and in combination with the chemotherapeutic drug gemcitabine in a clonogenic assay;

FIG. 17 shows the suppression of growth of pancreatic cancer cells (MiaPaCa) by compounds 3, 4, and 1 as single agents and in combination with the chemotherapeutic drug gemcitabine in a clonogenic assay;

FIGS. 18A and 18B demonstrate that compounds 1 and 3 inhibit NRF2 signaling in lung cancer cells. (A-B) A549 cells were treated with compound 1 (A) or compound 3 (B) for 48 h and fold change in mRNA was measured by real time RT-PCR. Data represent ±SD;

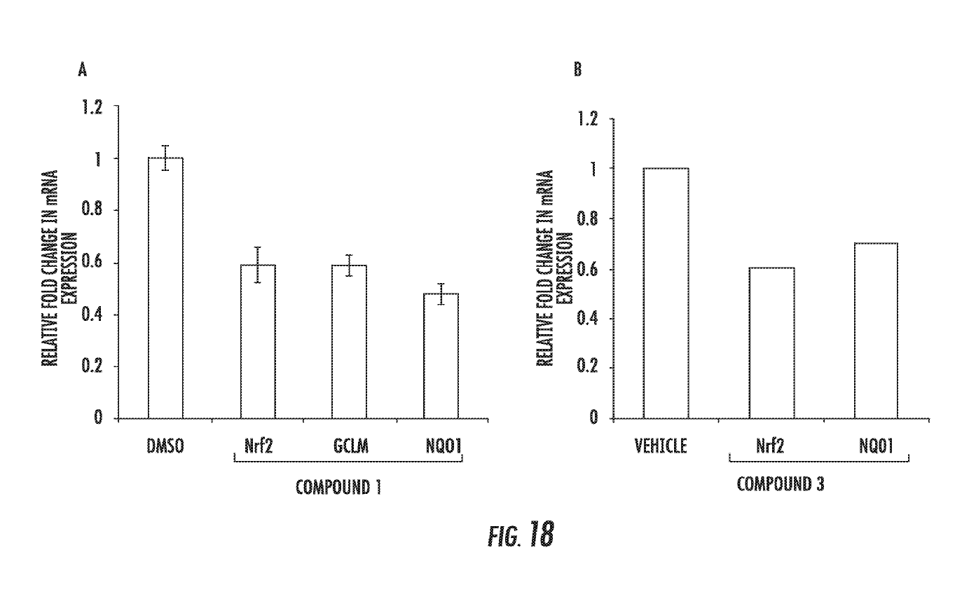

FIGS. 19A and 19B demonstrate that compound 4 inhibits NRF2 signaling in lung cancer cells. (A) A549 cells were treated with different concentrations of ML385 for 72 h and fold change in mRNA was measured by real time RT-PCR. Data represent ±s.e.m. (B) Time dependent reduction in NRF2 and its target genes following treatment with ML385 (5 μM);

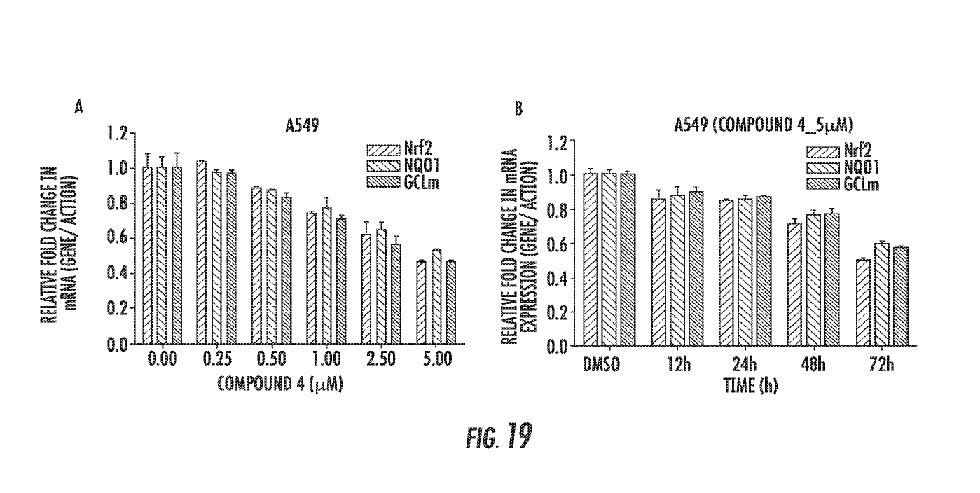

FIGS. 20A-20D demonstrate that compound 4 is selectively toxic to cells with KEAP1 mutations and potentiates the toxicity of standard care chemotherapy drugs in NSCLC cells with KEAP1 mutations. (A) H460, a NSCLC with a point mutation in KEAP1, is more sensitive to compound 4 than H460-KEAP1 Knock-in H460 cells expressing WT KEAP1. The cells were incubated with the inhibitor for 48 h. Colonies were stained with crystal violet staining and manually counted. (B-C) A549 and H460 cells were treated with different concentrations of paclitaxel, doxorubicin and carboplatin singly or in combination with compound 4 for 72 h. At the end of treatment, regular growth media was added and cells were further incubated for 8-10 days and stained with crystal violet. (D) H460 cells treated with a combination of compound 4 and chemo drug showed increased Caspase 3/7 activity, a marker of apoptosis. Cells treated with Chemo drug alone or a combination of compound 4 and chemo drug were incubated with luminogenic caspase substrate and change in luminescence was measured. Caspase activity was normalized to the number of viable cells using CellTiter-Blue assay. These data demonstrate the selectivity of compound 4, in that it is toxic to cancer cells, but less toxic to normal cells;

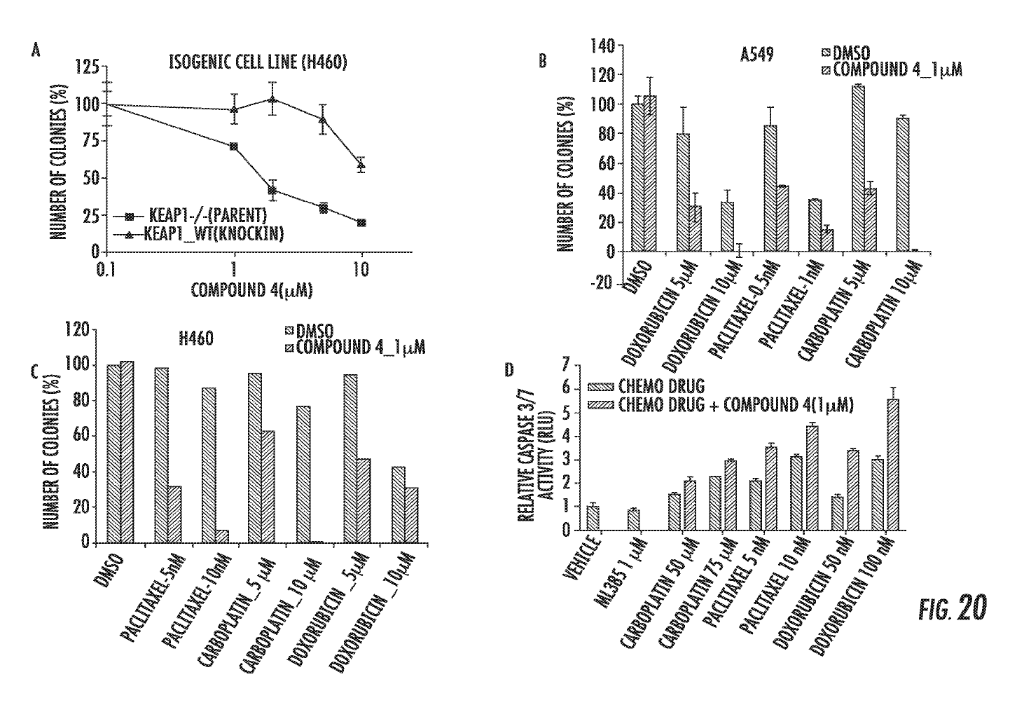

FIGS. 21A-21C show fluorescence spectroscopy (with tyrosine excitation). (A-B) Fluorescence spectrum of purified-NRF2 protein treated with the indicated concentration of compound 1 and 4 were measured. (excitation wavelength; 274 nm). (C) The peak heights of each curves were plotted on compound #1 and 4 concentration and EC₅₀ was calculated with non-linear curve fitting (R²>0.99);

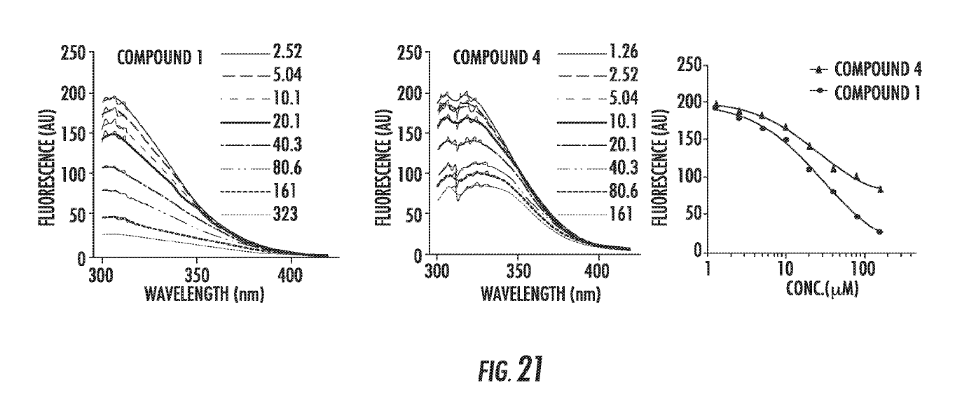

FIGS. 22A and 22B demonstrate that compound 4 shows significant growth inhibitory activity as single agent and further potentiates the cytotoxicity of standard care chemotherapy drugs (doxorubicin and etoposide) in sarcoma cells. (A) Ewing sarcoma cells (A4573) cells were on soft agar and treated with doxorubicin (2 nM) singly or in combination with compound 4 (10 μM) for 72 h. At the end of treatment, regular growth media was added and cells were further incubated for 8-10 days and colonies were stained with crystal violet and counted. (B) Rhabdomyosarcoma cells (Rh30) cells were on soft agar and treated with doxorubicin (2 nM) and etoposide (5-nM) singly or in combination with compound 4 (10 μM) for 72 h. At the end of treatment, regular growth media was added and cells were further incubated for 8-10 days and colonies were stained with crystal violet and counted;

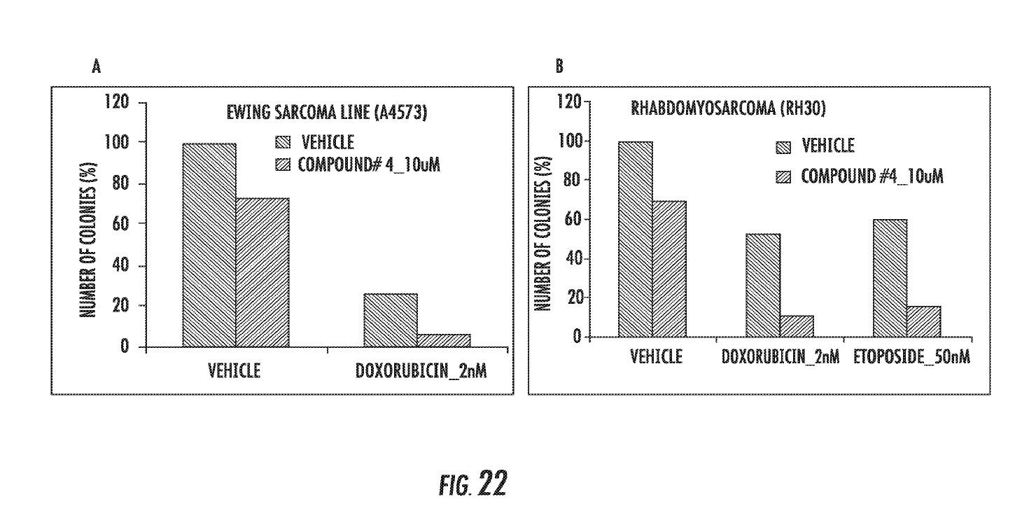

FIGS. 23A-23C demonstrate that NRF2 binds to biotin labeled compound 4. (A) Biotin labeled compound 4 (para and meta position) inhibits NRF2 promoter fig.construct (A549-ARE_Luciferase) was treated with different concentrations of compound 4 (5-10 μM) or biotin labeled compound 4 (5-20 μM) for 48 h. Relative luciferase activity was measured at 48 hr post treatment. Firefly luciferase activity was normalized to viable cells using CellTiter-Blue assay. Data represent ±SD. (B) Time dependent reduction in NRF2 and its target genes following treatment with compound 4 (5 μM) or biotin labeled analogs of compound 4 (10 μM). (C) Affinity purified Histidine tagged NRF2 protein (using Nickel resin) was incubated with DMSO (vehicle), free biotin, parent compound 4 (50 μM) or biotin labeled analogs of compound 4 (50 μM) for 2 hr and then washed with phosphate buffer. Chemiluminiscence was measured using streptavidin HRP antibody;

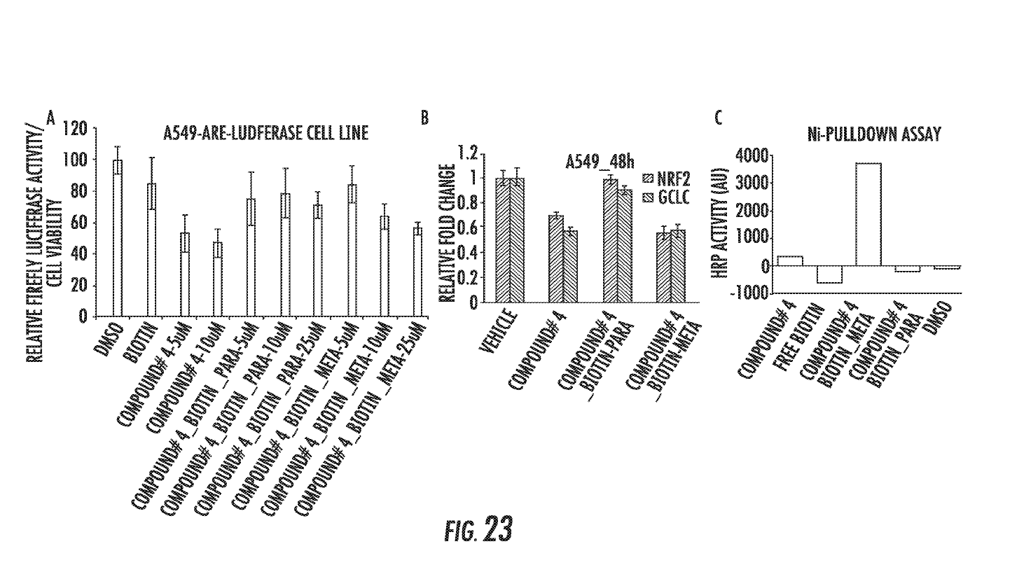

FIGS. 24A-24C demonstrate the therapeutic efficacy of compound 4 as a single agent and in combination with carboplatin in NSCLC lung tumors xenografts (subcutaneous and orthotopic model). (A-B) Compound 4 shows anti-tumor activity as a single agent and sensitized A549 lung tumors to carboplatin therapy. A549 cells were injected in the flanks of athymic nude mice and once tumor volume reached 50-100 mm³, treatment was initiated. Vehicle, carboplatin (5 mg/kg daily Monday to Friday), compound 4 (30 mg/kg daily Monday to Friday) or a combination of compound 4 and carboplatin was administered for three weeks. Values represent tumor volume ±s.e.m. for all groups (A). Treatment with ML385 or ML385+carboplatin significantly reduced tumor weight as compared to vehicle group. Efficacy of ML385 alone was comparable to carboplatin. Tumors were excised and weight at the end of treatment period. (C) Compound 4 shows anti-tumor efficacy as a single agent and in combination with carboplatin in an orthotopic lung tumor model. Bar graph showing tumor free lung volume in the carboplatin, compound 4 and combination therapy (carboplatin and compound 4) group at three weeks post-treatment suggesting that combination therapy is more effective in reducing tumor growth. For each group, pretreatment available lung volume was defined as 100% and compared with post-treatment lung volumes. Mice in the vehicle treated group died at 3 weeks;

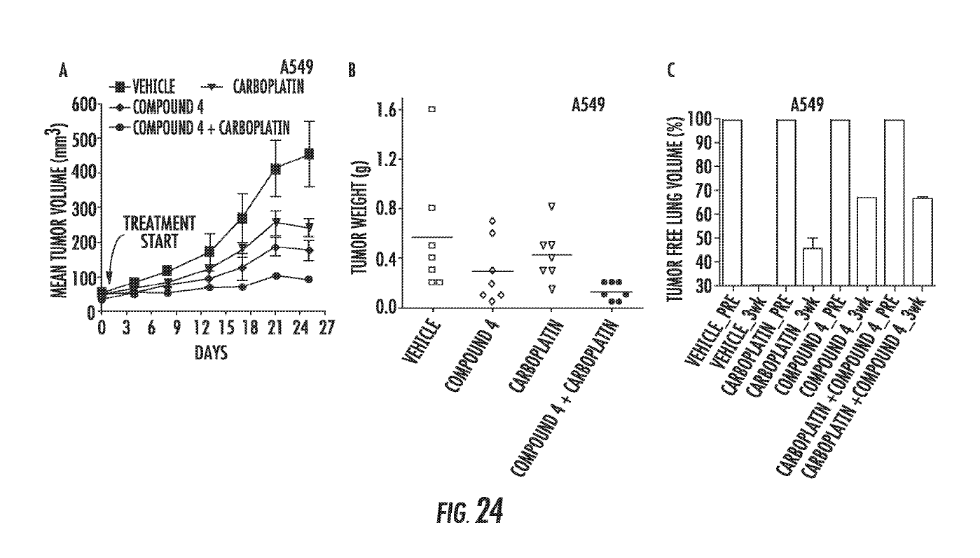

FIGS. 25A-25C demonstrate that compound 4 inhibits the growth of NSCLC lung tumors xenografts in both subcutaneous and orthotopic model (large cell model). (A-B) Compound 4 sensitized H460 lung tumors to carboplatin drug therapy. Groups of H460 tumors treated with ML385 or ML385+carboplatin showed significant reduction in tumor volume and weight as compared to the vehicle group. Efficacy of ML385 alone was comparable to carboplatin. (C) Compound 4 shows anti-tumor efficacy as a single agent and in combination with carboplatin in an orthotopic lung tumor model. Bar graph showing tumor free lung volume in the carboplatin, compound 4 and combination therapy (carboplatin and compound 4) group at two weeks post-treatment suggesting that combination therapy is more effective in reducing tumor growth. For each group, pretreatment available lung volume was defined as 100% and compared with post-treatment lung volumes. Mice in the vehicle treated group did not survive for 2 weeks;

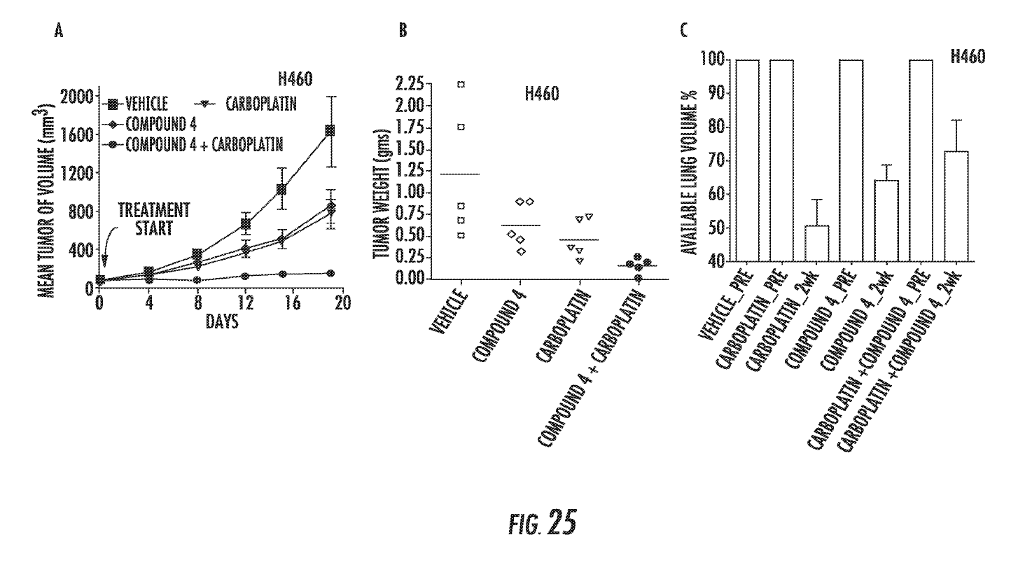

FIGS. 26A-26C demonstrate that compound 1 inhibits the growth of NSCLC lung tumors xenografts. (A-B) Compound 1 shows anti-tumor activity as a single agent and sensitized A549 lung tumors to carboplatin therapy. A549 cells were injected in the flanks of athymic nude mice and once tumor volume reached 50-100 mm³, treatment was initiated. Vehicle, carboplatin (20 mg/kg; 2 days/week), compound#1 (20 mg/kg 4 days/week) or a combination of compound 1 and carboplatin was administered for four weeks. Values represent tumor volume ±s.e.m. for all groups. (B) Treatment with compound 1 or compound 1+carboplatin significantly reduced tumor weight as compared to vehicle group. Efficacy of compound#1 alone was comparable to carboplatin. Tumors were excised and weight at the end of treatment period. (C) In H460 cells, Compound 1 shows anti-tumor efficacy in combination with carboplatin in an orthotopic lung tumor model. H460 cells were injected in the flanks of athymic nude mice and once tumor volume reached 50-100 mm³, treatment was initiated. Vehicle, carboplatin (10 mg/kg; 5 days/week), compound 1 (30 mg/kg 5 days/week) or a combination of compound 1 and carboplatin was administered for three weeks. Values represent tumor volume ±s.e.m.;

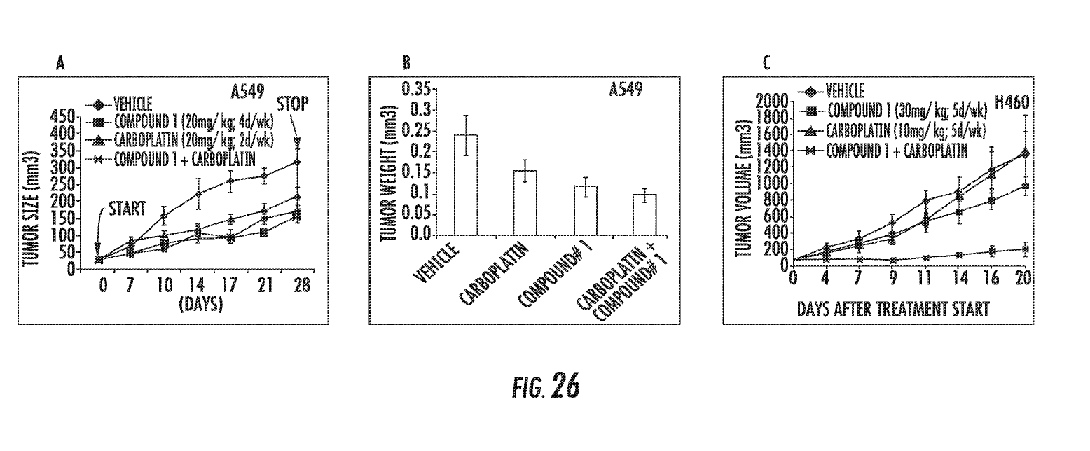

FIGS. 27A and 27B demonstrate that compound 3 inhibits the growth of NSCLC lung tumors xenografts. (A-B) Compound 3 shows anti-tumor activity as a single agent and sensitized A549 lung tumors to carboplatin therapy. A549 cells were injected in the flanks of athymic nude mice and once tumor volume reached 50-100 mm³, treatment was initiated. Vehicle, carboplatin (10 mg/kg; 5 days/week), compound 3 (60 mg/kg 4 days/week) or a combination of compound 3 and carboplatin was administered for 15 days. Tumor growth was monitored till 28 days. Values represent tumor volume ±s.e.m. for all groups. Treatment with compound 3 or compound 3+carboplatin significantly attenuated tumor growth till 28 days even though drug treatment was stopped at 14 days. Efficacy of compound 3 alone was comparable to carboplatin. (B) At Day 43, 4 weeks from the end of treatment period, A549 tumor group treated with combination of Compound 3 and carboplatin showed significant growth retardation as compared to group treated with single agent. Values represent tumor volume ±s.e.m.;

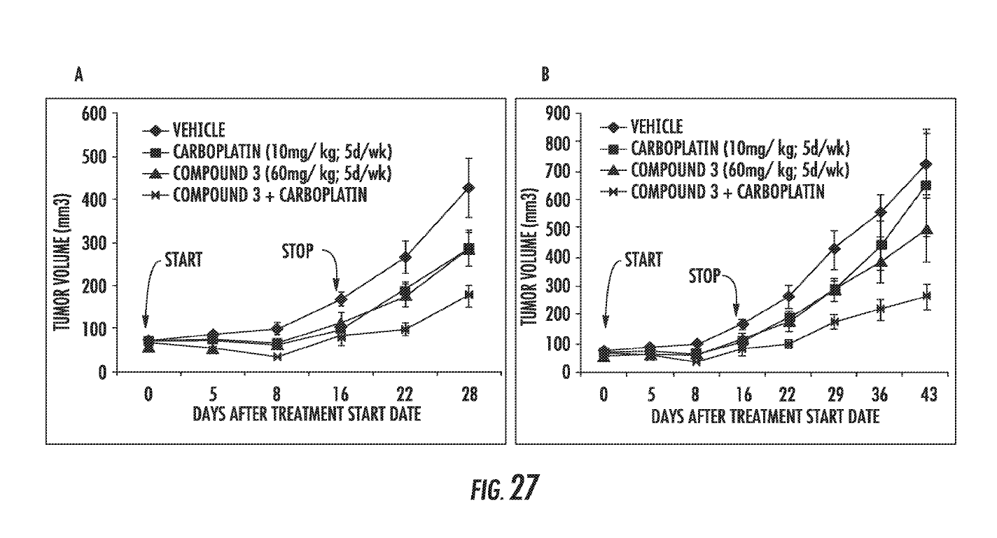

FIGS. 28A and 28B demonstrate that compound 1 potentiates the cytotoxicity of standard care chemotherapy drugs (gemcitabine) in Pancreatic cancer cells. (A-B) Panc1 and MiaPaCa cells were treated gemcitabine (10 nM) singly or in combination with compound 1 for 72 h. At the end of treatment, regular growth media was added and cells were further incubated for 8-10 days and stained with crystal violet;

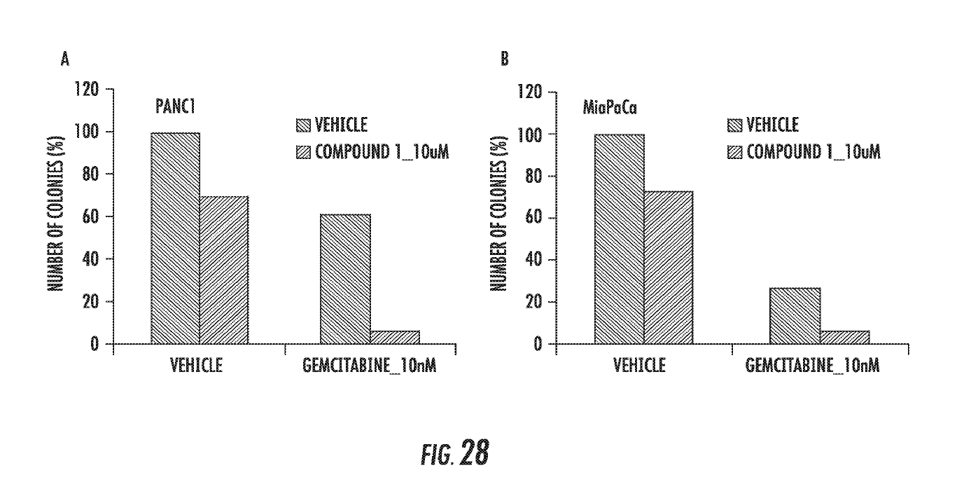

FIGS. 29A and 29B demonstrate that compound 4 shows significant growth inhibitory activity as single agent and further potentiates the cytotoxicity of standard care chemotherapy drugs (gemcitabine) in Pancreatic cancer cells. (A-B) Panel and MiaPaCa cells were treated with gemcitabine (10 nM) singly or in combination with compound 4 for 72 h. At the end of treatment, regular growth media was added and cells were further incubated for 8-10 days and stained with crystal violet; and

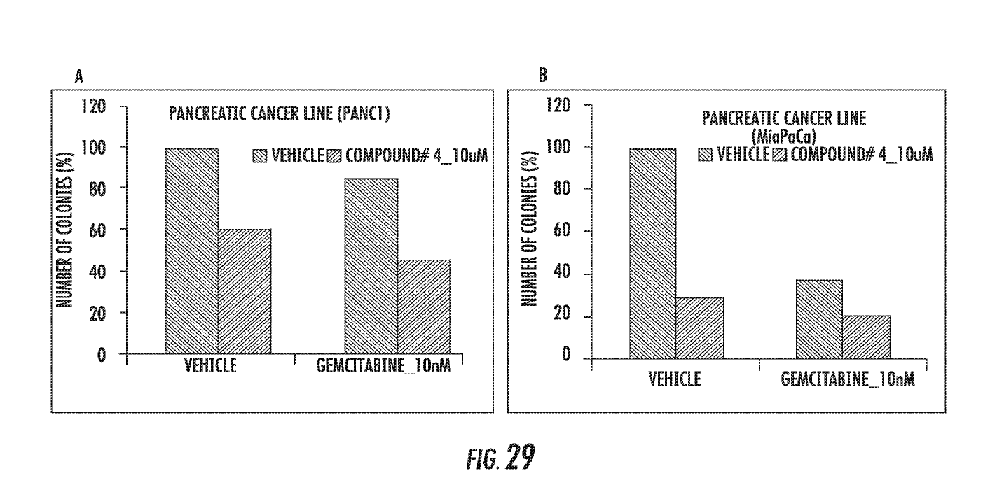

FIGS. 30A and 30B demonstrate that compound 3 shows significant growth inhibitory effect as a single agent in pancreatic cancer cells. (A-B) Panel and MiaPaCa cells were treated with compound 3 (10 μM) for 72 h. At the end of treatment, regular growth media was added and cells were further incubated for 8-10 days and stained with crystal violet.

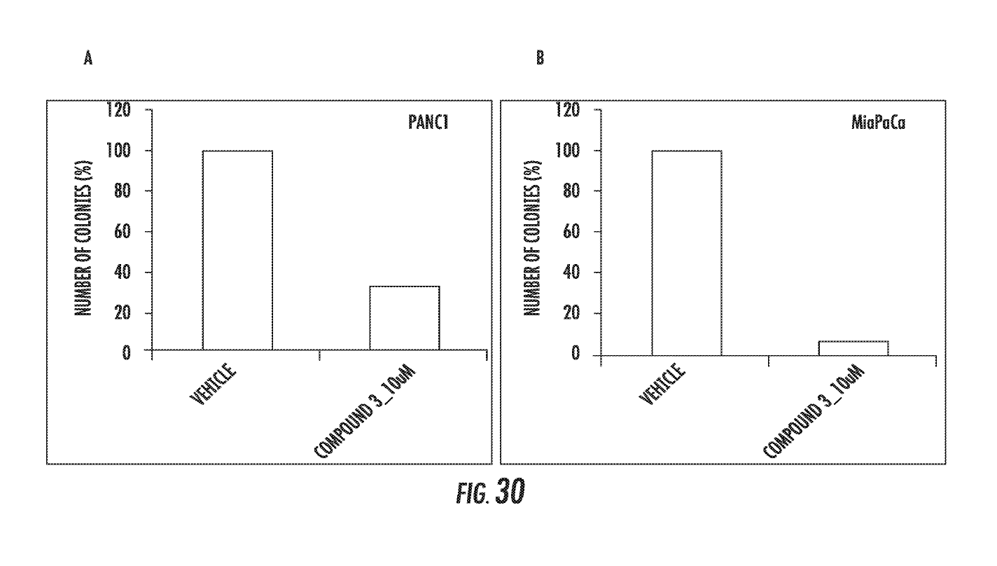

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Inhibition of Nuclear Factor Erythroid-2 Related Factor-2 (Nrf2)

Nuclear factor erythroid-2 related factor-2 (Nrf2) is a redox-sensitive transcription factor that regulates the expression of electrophile and xenobiotic detoxification enzymes and efflux proteins, which confer cytoprotection against oxidative stress and apoptosis in normal cells. Nrf2-mediated activation of antioxidant response element (ARE) is a central part of molecular mechanisms governing the protective function of phase II detoxification and antioxidant enzymes against oxidative stress and inflammation. By “Nrf2 polypeptide” is meant a protein or protein variant, or fragment thereof, that comprises an amino acid sequence substantially identical to at least a portion of GenBank Accession No. NPJ306164 (human nuclear factor (erythroid-derived 2)-like 2) and that has an Nrf2 biological activity (e.g., activation of target genes through binding to antioxidant response element (ARE), regulation of expression of antioxidants and xenobiotic metabolism genes).

Nrf2 is sequestered in the cytoplasm by its repressor, Keap1. The Keap1-Nrf2 system is the major regulatory pathway of cytoprotective gene expression against oxidative and/or electrophilic stresses. Upon activation in response to inflammatory stimuli, environmental toxicants, or oxidative and electrophilic stress, Nrf2 detaches from its cytosolic inhibitor, Kelch-like ECH-associated protein 1 (Keap1), and translocates to the nucleus and binds to the antioxidant response element (ARE) of target genes along with other binding partners leading to their transcriptional induction (Kensler et al., 2007; Rangasamy et al., 2005; Sussan et al., 2009).

Keap1 acts as a stress sensor protein in this system. While Keap1 constitutively suppresses Nrf2 activity under unstressed conditions, oxidants or electrophiles provoke the repression of Keap1 activity, thereby inducing the Nrf2 activation (Misra et al., 2007; Surh et al., 2008; Singh et al., 2008). Gain of Nrf2 function resulting from inactivating mutations in Keap1 or activating mutations in Nrf2 promotes tumorigenesis and confers therapeutic resistance.

In addition to Keap1 the activation of protein kinases, such as protein kinase C (PKC), extracellular signal-regulated kinases (ERK), p38 mitogen-activated protein kinase (MAPK), phosphatidylinositol 3-kinase (PI3K) and protein kinase RNA-like endoplasmic reticulum kinase (PERK), has been shown to activate Nrf2 (Niture et al., 2009; Cheng et al., 2010; Nioi et al., 2003; Chartoumpekis et al., 2010; Cullinan et al., 2004).

Modification of cysteine residues in Keap1 by a variety of inducers, specifically Michael acceptors, results in a conformational change that renders Keap1 to dissociate from Nrf2, thereby inducing translocation of Nrf2 to the nucleus. By “Keap1 polypeptide” is meant a polypeptide comprising an amino acid sequence having at least 85% identity to GenBank Accession No. AAH21957. By “Keap1 nucleic acid molecule” is meant a nucleic acid molecule that encodes a Keap1 polypeptide or fragment thereof.

Representative Nrf2-regulated gene functions are summarized in Table 1.

TABLE 1
NRF2-regulated Gene Functions

| Target Genes | Functions |
|---|---|
| Heme oxygenase-1, Ferritin, | Direct antioxidants |
| NQO1, SOD1 | |
| GCLM, GCLC, GCS, GSR | Increase the levels of GSH synthesis and regeneration |
| G6PD, malic enzyme, PGD | Stimulate NADPH synthesis |
| GSTs, UGTs | Encode enzymes that directly inactivate oxidants or |
| | electrophiles |
| GPX2, peroxiredoxin, | Increases detoxification of H₂O₂, peroxynitrite, and |
| catalase, sulfiredoxin | oxidative damage by products (4HNE, lipid |
| | hydroperoxides); |
| | Enhance the recognition and repair and removal of |
| | damaged DNA |
| Heat shock proteins (HSP | Chaperone activity; Enhance the recognition, repair, |
| 70), Proteosome members | and removal of damaged proteins |
| MRP1, MRP2, MRP3, | Enhance drug/toxin efflux via the multidrug response |
| MRP4, MRP10, ABCG2 | transporters |
| Leukotriene B4 12- | Inhibits cytokine mediated inflammation |
| hydroxydehydrogenase | |
| CD36, MARCO (scavenger | i) Enhances phagocytosis of bacteria |
| receptors) | ii) Maintenance of tissue homeostasis and resolution |
| | of inflammatory lesions by clearance of apoptotic |
| | cells |
| Suppress NF-KB signaling | Regulates redox dependent innate immune, as well as |
| | adaptive immune response |

Cancer cells show greater expression of drug detoxification enzymes and efflux pumps. This characteristic can result in cancer therapeutic resistance due to the ability of a cancer cell to eliminate a toxic drug, such as a chemotherapeutic drug, from the cell. It has been found that a gain in Nrf2 function can be a major factor for cancer therapeutic resistance in various cancers. Further, it has been shown that a decrease of Nrf2 expression leads to sensitization of cells against ionizing radiation, such as the type of radiation used in radiation therapy.

In some embodiments, the presently disclosed subject matter provides compositions and methods to modulate Nrf2 expression using small molecule Nrf2 inhibitors. In particular embodiments, the presently disclosed subject matter provides compositions and methods to decrease Nrf2 expression using small molecule Nrf2 inhibitors.

In further embodiments, the presently disclosed subject matter provides methods using combination therapy of the presently disclosed Nrf2 inhibitors and commonly used chemotherapeutic drugs to effectively downregulate expression of chemoresistance genes and reduce drug resistance in cancers, which remains one of the greatest challenges in improving the efficacy of cancer therapy. The presently disclosed Nrf2 inhibitors also can be used as an adjuvant or in combination with radiation therapy.

Accordingly, the Nrf2 inhibitors of the presently disclosed subject matter can be exploited to combat chemoresistance and radioresistance and used as adjuvant or in combination with chemotherapeutic drugs and/or radiation. In addition, the presently disclosed inhibitors also can be used as single agent adjuvants, such as single agent adjuvant post surgery in management of patients with early stage cancer.

In some embodiments, the presently disclosed subject matter provides Nrf2 inhibitors that decrease Nrf2 transcription, translation, and/or biological activity. As provided in more detail herein below, the presently disclosed compounds can be used for treating or preventing diseases, disorders, or conditions associated with Nrf2-regulated pathways, including, but not limited to an autoimmune disease, comorbidity associated with diabetes, such as retinopathy and nephropathy, bone marrow transplant for leukemia and related cancers, bone marrow deficiencies, inborn errors of metabolism, and other immune disorders, oxidative stress, respiratory infection, ischemia, neurodegenerative disorders, radiation injury, neutropenia caused by chemotherapy, autoimmunity, congenital neutropenic disorders, and cancer.

These Nrf2 inhibitors can be used with different kinds of chemotherapeutic drugs to combat chemoresistance and radioresistance and can be used as adjuvant or in combination with chemotherapeutic drugs or radiation therapy. Therefore, the presently disclosed subject matter is particularly applicable to diseases, disorders, or conditions that use chemotherapeutic drugs and/or radiation therapies as a treatment method.

By “in combination with” is meant the administration of a presently disclosed compound with one or more therapeutic agents either simultaneously, sequentially, or a combination thereof. Therefore, a cell or a subject administered a combination of a presently disclosed compound can receive a another type of presently disclosed compound and one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where a presently disclosed compound and one or more therapeutic agents are administered simultaneously, they can be administered to the cell or administered to the subject as separate pharmaceutical compositions, each comprising either a presently disclosed compound or one or more therapeutic agents, or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

The term “ionizing radiation” refers to radiation composed of particles that individually carry enough energy to liberate an electron from an atom or molecule without raising the bulk material to ionization temperature. Ionizing radiation is used in radiation therapy, which is the medical use of ionizing radiation, generally as part of a treatment to control or kill malignant cells.

The term “chemotherapy” refers to the treatment of disease by the use of chemical substances. For example, these diseases, disorders, or conditions generally include various cancers. Accordingly, in a particular embodiment, the presently disclosed subject matter can be used for many different types of cancer, such as common cancers like lung, ovarian, breast, prostate, head and neck, skin, renal and brain, hematological malignancies (leukemia, lymphoma, myeloma) as well as orphan cancers such as sarcoma, gall bladder, liver, and pancreatic cancers.

“Cancer” is defined herein as a disease caused by an uncontrolled division of abnormal cells in a part of the body. Over time, cancer cells become more resistant to chemotherapy and radiation treatments. The presently disclosed compounds and methods aid in making cancer cells less resistant to chemotherapy and/or radiation therapy.

Chemotherapeutic drugs include alkylating agents, antimetabolites, anthracyclines, plant alkoids, topoisomerase inhibitors, and other antitumor agents. These drugs affect DNA synthesis, DNA function, or cell division in some way. Examples of chemotherapeutic drugs include cisplatin (cisplatinum, cis-diamminedichloroplatinum (II)), carboplatin (1,1-cyclobutanedicarboxylato)-platinum(II)), oxaliplatin, mechlorethamine, cyclophosphamide, chlorambucil, ifosfamide, azathioprine, mercaptopurine, vincristine, vinblastine, vinorelbine, vindesine, etoposide (etoposide phosphate, VP-16), teniposide, paclitaxel (taxane), docetaxel, irinotecan, topotecan, amsacrine, actinomycin, doxorubicin, daunorubicin, valrubicin, idarubicin, epirubicin, bleomycin, plicamycin, lapatinib, sorafenib, gemcitabine, and mitomycin.

“Adjuvant” herein refers to an agent that modifies the effect of other agents. Therefore, in some embodiment, the compositions and methods of the presently disclosed subject matter can be used as adjuvants along with chemotherapeutic drugs or radiation therapy to make diseased cells in a subject less resistant to the drugs or radiation.

A. Compositions Comprising the Presently Disclosed Nrf2 Inhibitors

Compositions comprising the presently disclosed Nrf2 inhibitors can effectively downregulate expression of chemoresistance and radioresistance genes and reduce drug and radiation resistance in a subject.

Accordingly, in some embodiments, the presently disclosed subject matter provides compound selected from the group consisting:

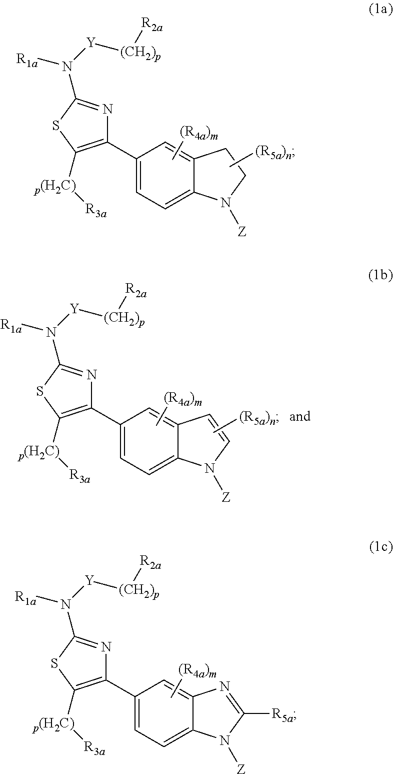

wherein:

m is an integer selected from the group consisting of 0, 1, 2, and 3;

n is an integer selected from the group consisting of 0, 1, and 2;

each p is independently an integer selected from the group consisting of 0, 1, and 2;

R₁ₐ is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R₂ₐ is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

R₃ₐ is selected from the group consisting of H and substituted or unsubstituted straight-chain or branched alkyl;

each R₄ₐ and R₅ₐ is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R₆ₐ is selected from the group consisting of H, substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

Y is —(C═O)— or —S(═O)₂—; and

Z is selected from the group consisting of R₆ₐ, —C(═O)—(CH₂)ₚ—R₆ₐ, and —S(═O)₂—R₆ₐ; p is an integer selected from the group consisting of 0, 1, and 2;

or an enantiomer, diastereomer, racemate or pharmaceutically acceptable salt, prodrug, or solvate thereof;

under the proviso that the compound of formula (1a) is not a compound selected from the group consisting of:

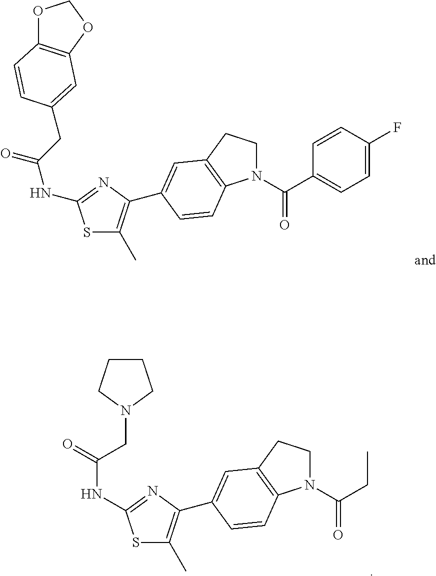 [SMILES: Cc1sc(NC(=O)Cc2ccc3c(c2)OCO3)nc1-c1ccc2c(c1)CCN2C(=O)c1ccc(F)cc1 ; CCC(=O)N1CCc2cc(-c3nc(NC(=O)CN4CCCC4)sc3C)ccc21]

Accordingly, the presently disclosed compounds are subject to the proviso that they do not include compounds disclosed in U.S. Patent Application Publication No. US2009/0163545 for METHOD FOR ALTERING THE LIFESPAN OF EUKARYOTIC ORGANISMS, to Goldfarb, published Jun. 25, 2009; International PCT Patent Application Publication No. WO2009/086303 for METHOD FOR ALTERING THE LIFESPAN OF EUKARYOTIC ORGANISMS, to Goldfarb, published Jul. 7, 2009; International PCT Patent Application Publication No. WO2011/062964 for COMPOUNDS AND METHODS FOR ALTERING LIFESPAN OF EUKARYOTIC ORGANISMS, to Goldfarb, published May 26, 2011; U.S. Patent Application Publication No. 20130096175 for COMPOUNDS AND METHODS FOR ALTERING LIFESPAN OF EUKARYOTIC ORGANISMS, to Goldfarb, published Apr. 18, 2013; and International PCT Patent Application Publication No. WO2007/076055 for COMPOSITIONS AND METHODS COMPRISING PROTEINASE ACTIVATED RECEPTOR ANTAGONISTS to Hembrough et al., published Jul. 5, 2007, each of which is incorporated herein in its entirety.

In some embodiments, the compound is a compound of Formula (1a′):

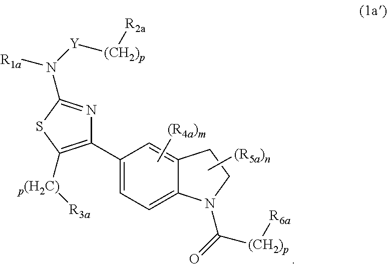

In particular embodiments, the compound of formula (1a′) is selected from the group consisting of:

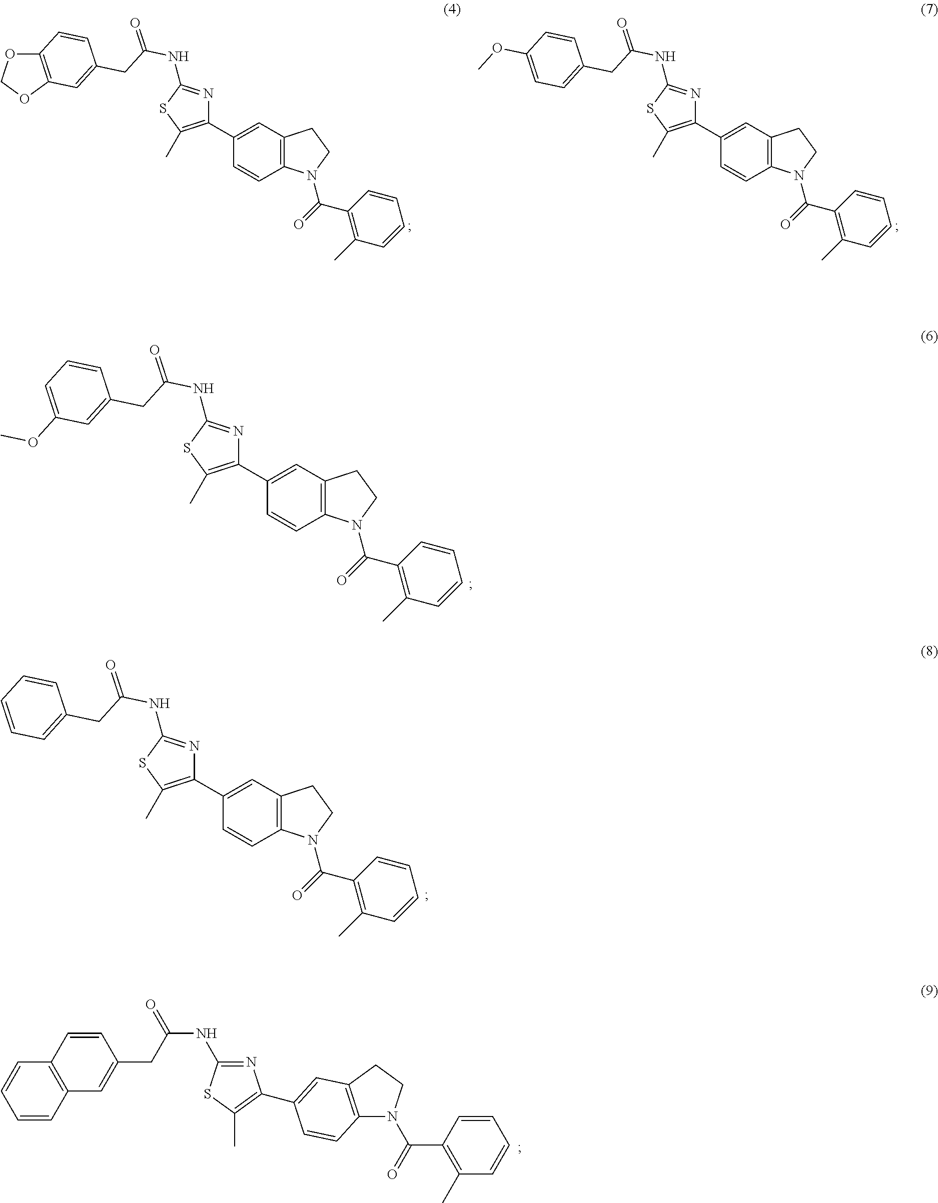 [SMILES: Cc1ccccc1C(=O)N1CCc2cc(-c3nc(NC(=O)Cc4ccc5c(c4)OCO5)sc3C)ccc21 ; COc1ccc(CC(=O)Nc2nc(-c3ccc4c(c3)CCN4C(=O)c3ccccc3C)c(C)s2)cc1 ; COc1cccc(CC(=O)Nc2nc(-c3ccc4c(c3)CCN4C(=O)c3ccccc3C)c(C)s2)c1 ; Cc1ccccc1C(=O)N1CCc2cc(-c3nc(NC(=O)Cc4ccccc4)sc3C)ccc21 ; Cc1ccccc1C(=O)N1CCc2cc(-c3nc(NC(=O)Cc4ccc5ccccc5c4)sc3C)ccc21]

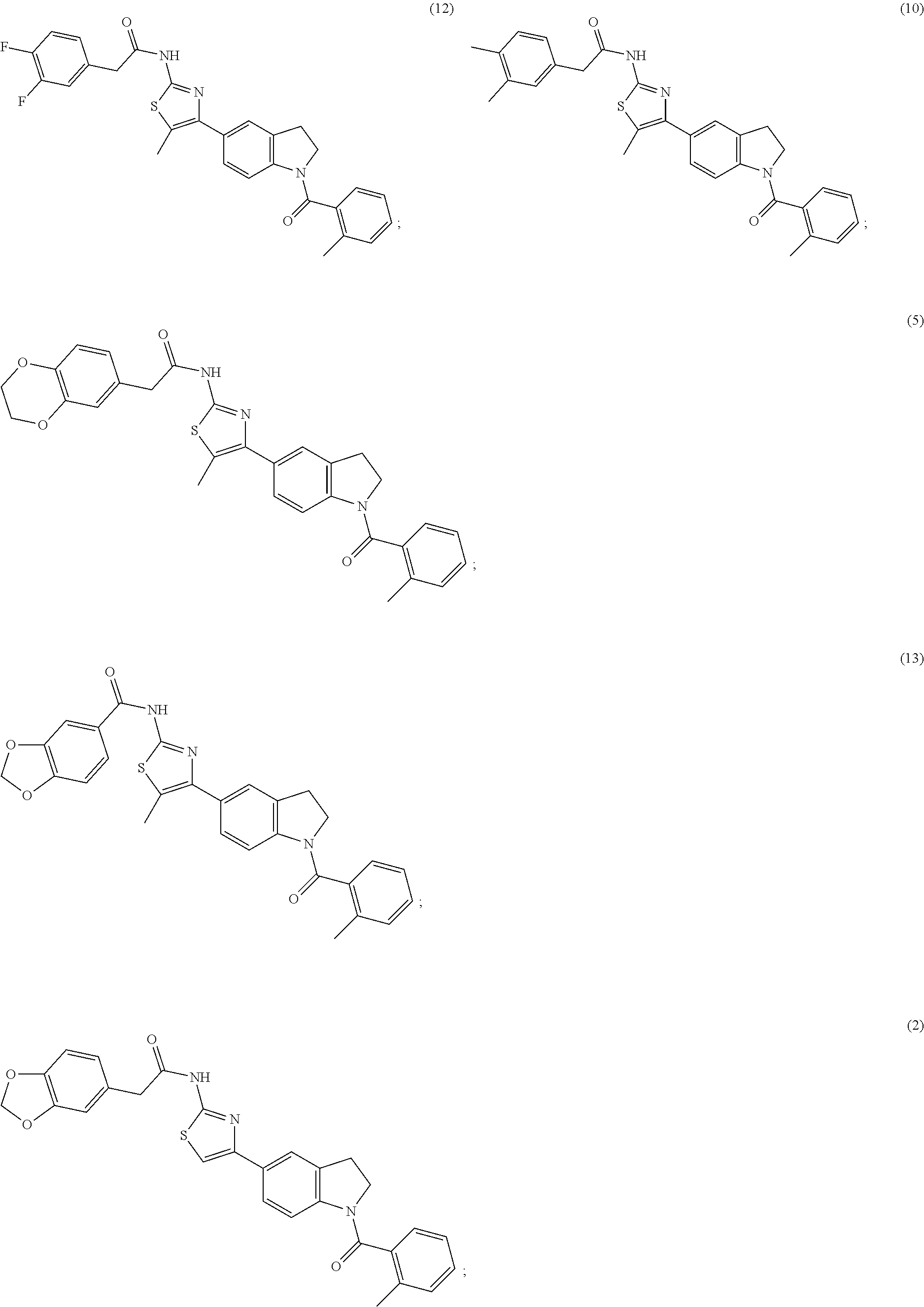 [SMILES: Cc1ccccc1C(=O)N1CCc2cc(-c3nc(NC(=O)Cc4ccc(F)c(F)c4)sc3C)ccc21 ; Cc1ccc(CC(=O)Nc2nc(-c3ccc4c(c3)CCN4C(=O)c3ccccc3C)c(C)s2)cc1C ; Cc1ccccc1C(=O)N1CCc2cc(-c3nc(NC(=O)Cc4ccc5c(c4)OCCO5)sc3C)ccc21 ; Cc1ccccc1C(=O)N1CCc2cc(-c3nc(NC(=O)c4ccc5c(c4)OCO5)sc3C)ccc21 ; Cc1ccccc1C(=O)N1CCc2cc(-c3csc(NC(=O)Cc4ccc5c(c4)OCO5)n3)ccc21]

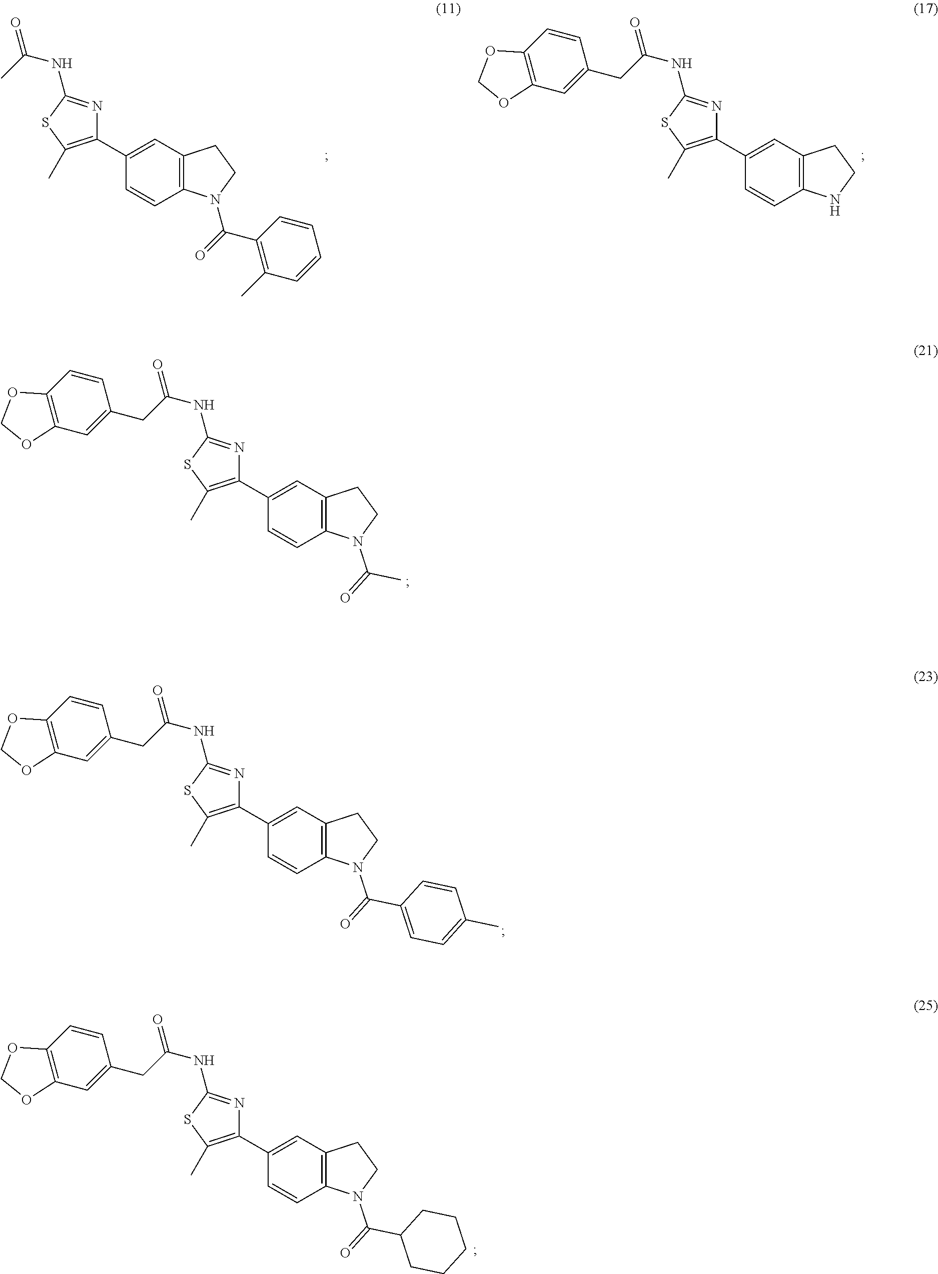 [SMILES: CC(=O)Nc1nc(-c2ccc3c(c2)CCN3C(=O)c2ccccc2C)c(C)s1 ; Cc1sc(NC(=O)Cc2ccc3c(c2)OCO3)nc1-c1ccc2c(c1)CCN2 ; CC(=O)N1CCc2cc(-c3nc(NC(=O)Cc4ccc5c(c4)OCO5)sc3C)ccc21 ; Cc1ccc(C(=O)N2CCc3cc(-c4nc(NC(=O)Cc5ccc6c(c5)OCO6)sc4C)ccc32)cc1 ; Cc1sc(NC(=O)Cc2ccc3c(c2)OCO3)nc1-c1ccc2c(c1)CCN2C(=O)C1CCCCC1]

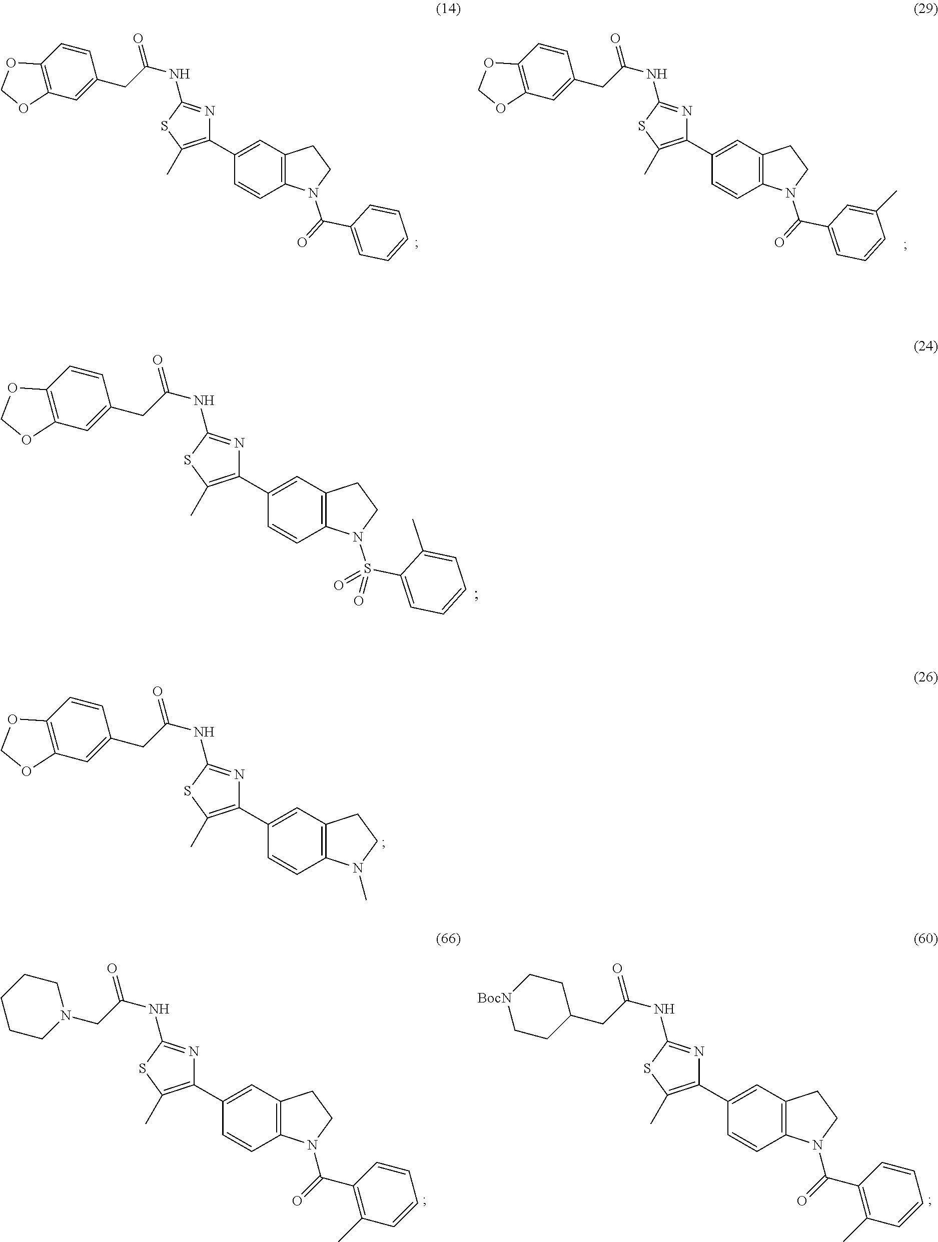 [SMILES: Cc1sc(NC(=O)Cc2ccc3c(c2)OCO3)nc1-c1ccc2c(c1)CCN2C(=O)c1ccccc1 ; Cc1cccc(C(=O)N2CCc3cc(-c4nc(NC(=O)Cc5ccc6c(c5)OCO6)sc4C)ccc32)c1 ; Cc1ccccc1S(=O)(=O)N1CCc2cc(-c3nc(NC(=O)Cc4ccc5c(c4)OCO5)sc3C)ccc21 ; Cc1sc(NC(=O)Cc2ccc3c(c2)OCO3)nc1-c1ccc2c(c1)CCN2C ; Cc1ccccc1C(=O)N1CCc2cc(-c3nc(NC(=O)CN4CCCCC4)sc3C)ccc21 ; Cc1ccccc1C(=O)N1CCc2cc(-c3nc(NC(=O)CC4CCN(C(=O)OC(C)(C)C)CC4)sc3C)ccc21]

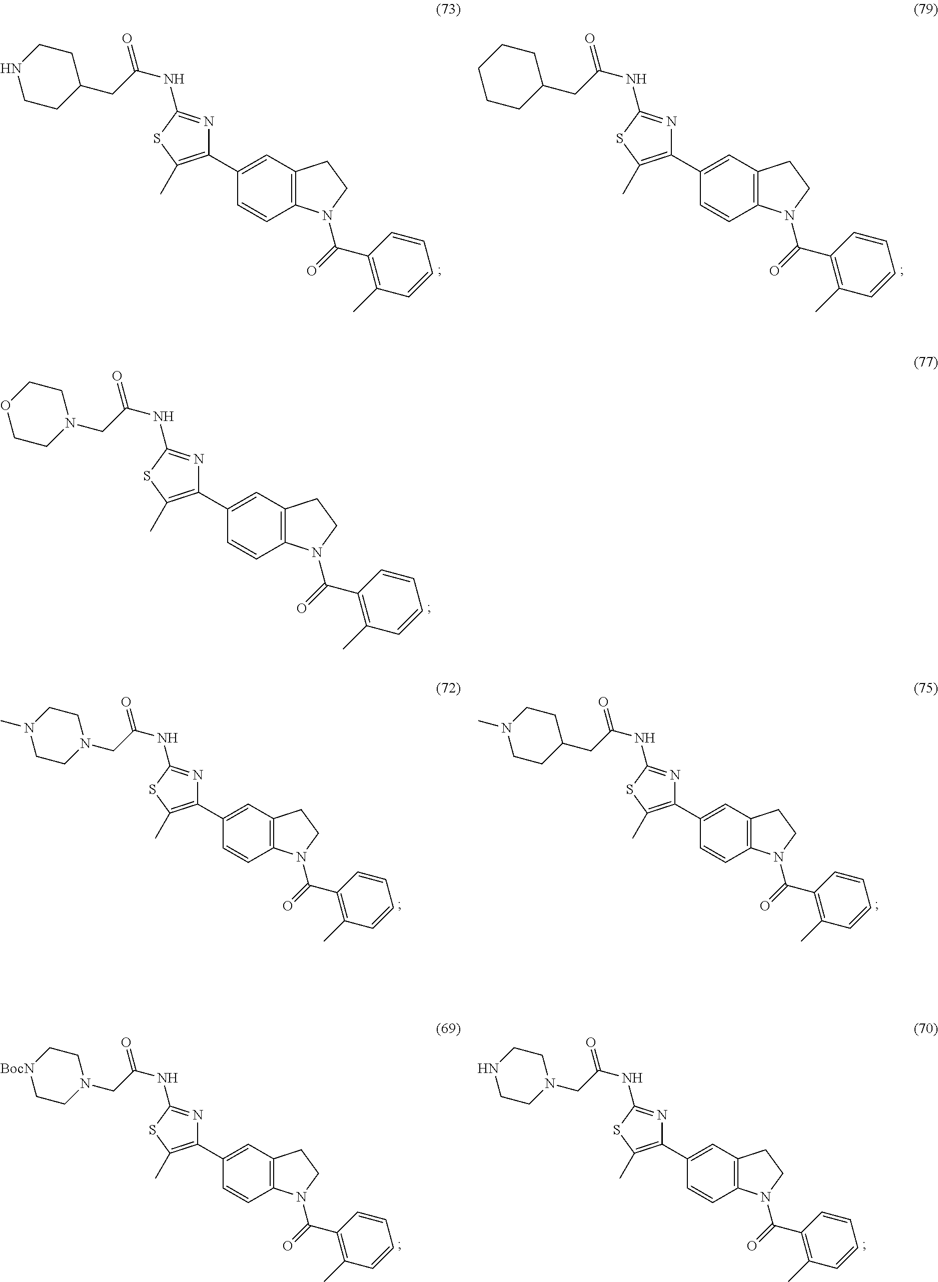

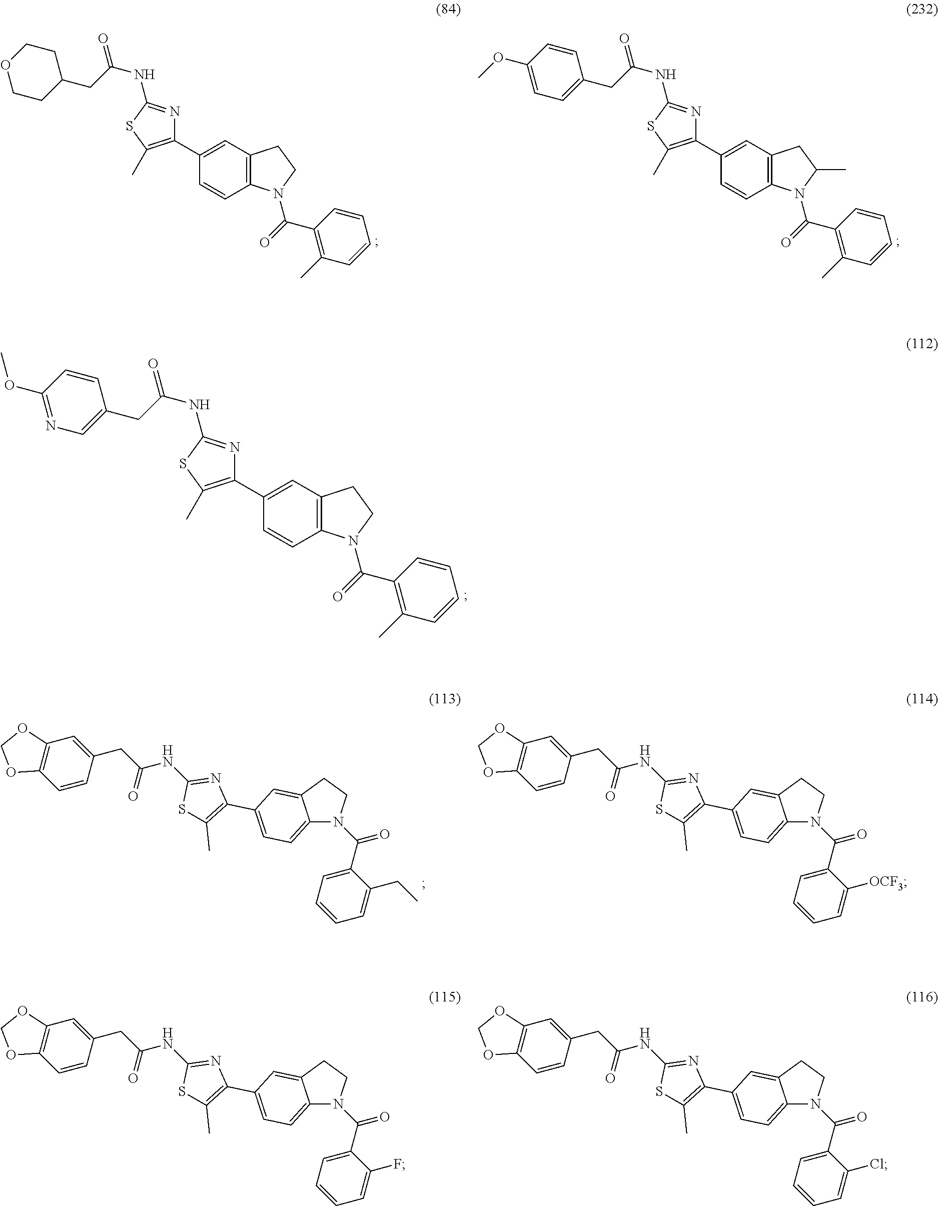

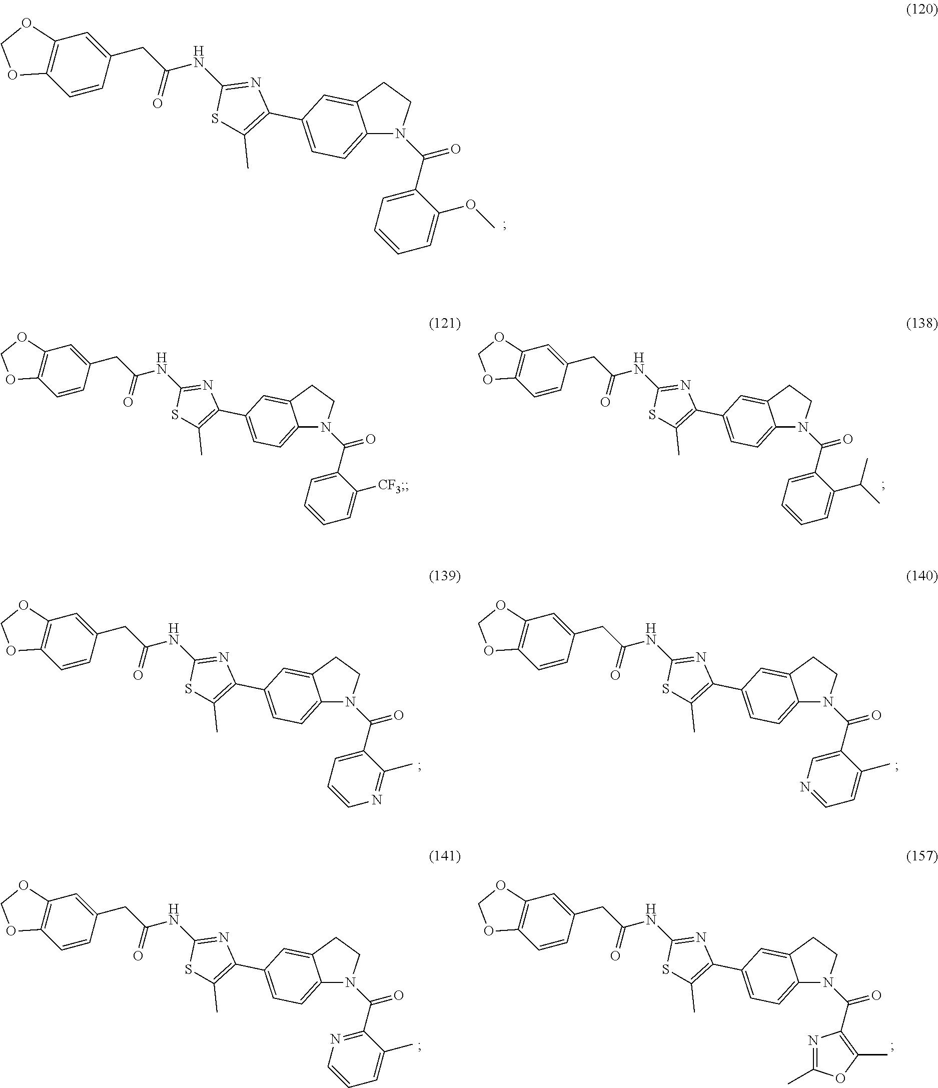

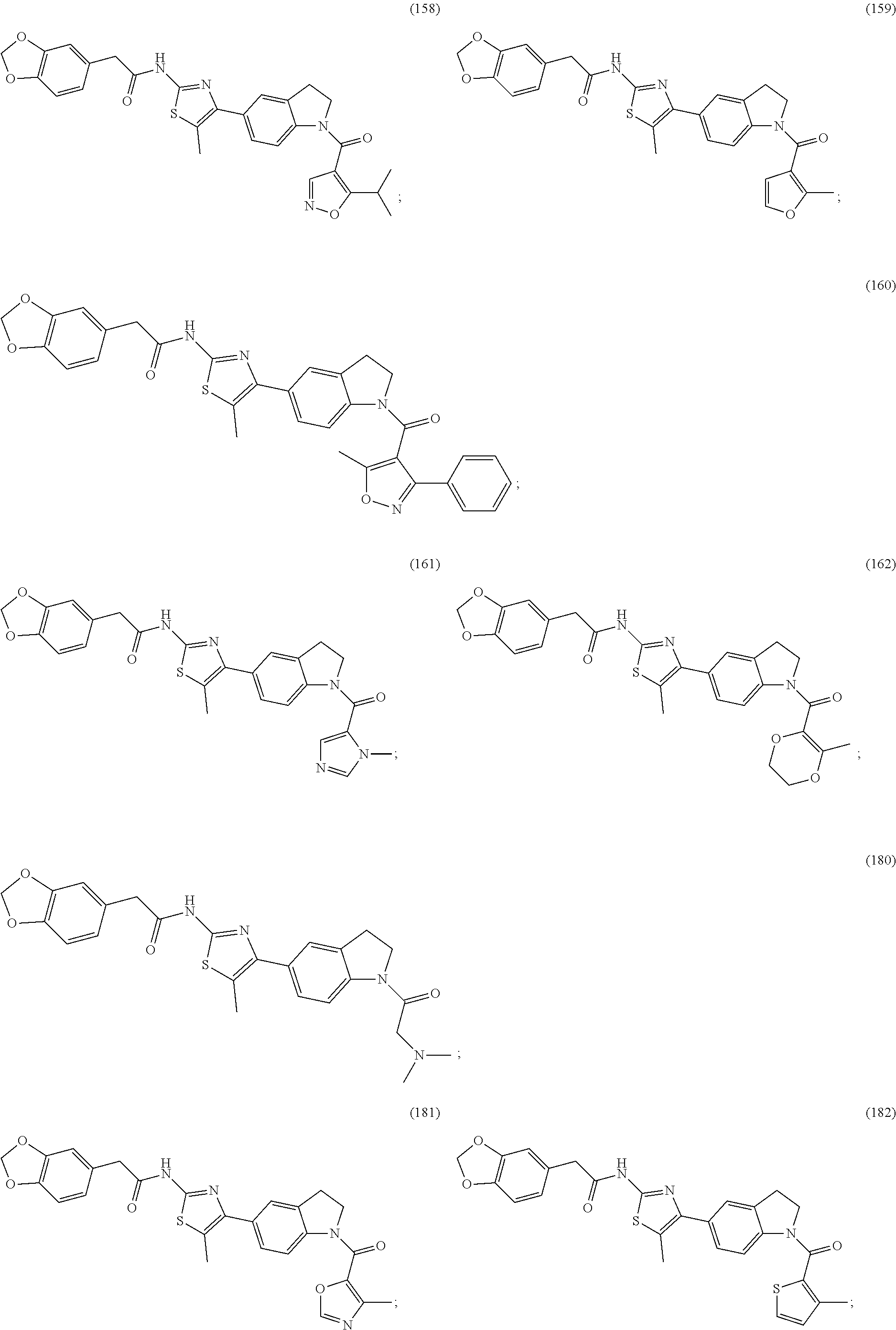

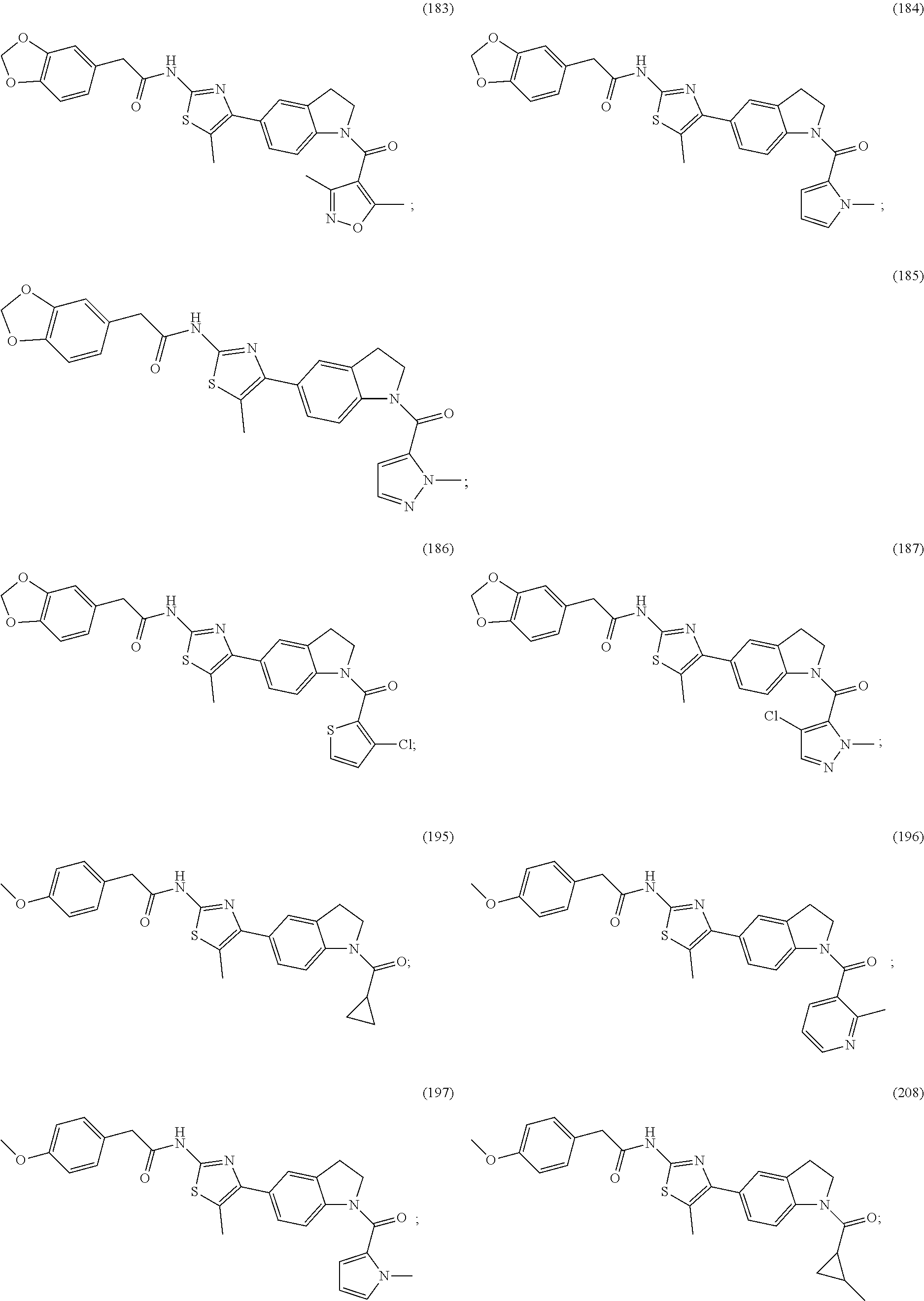

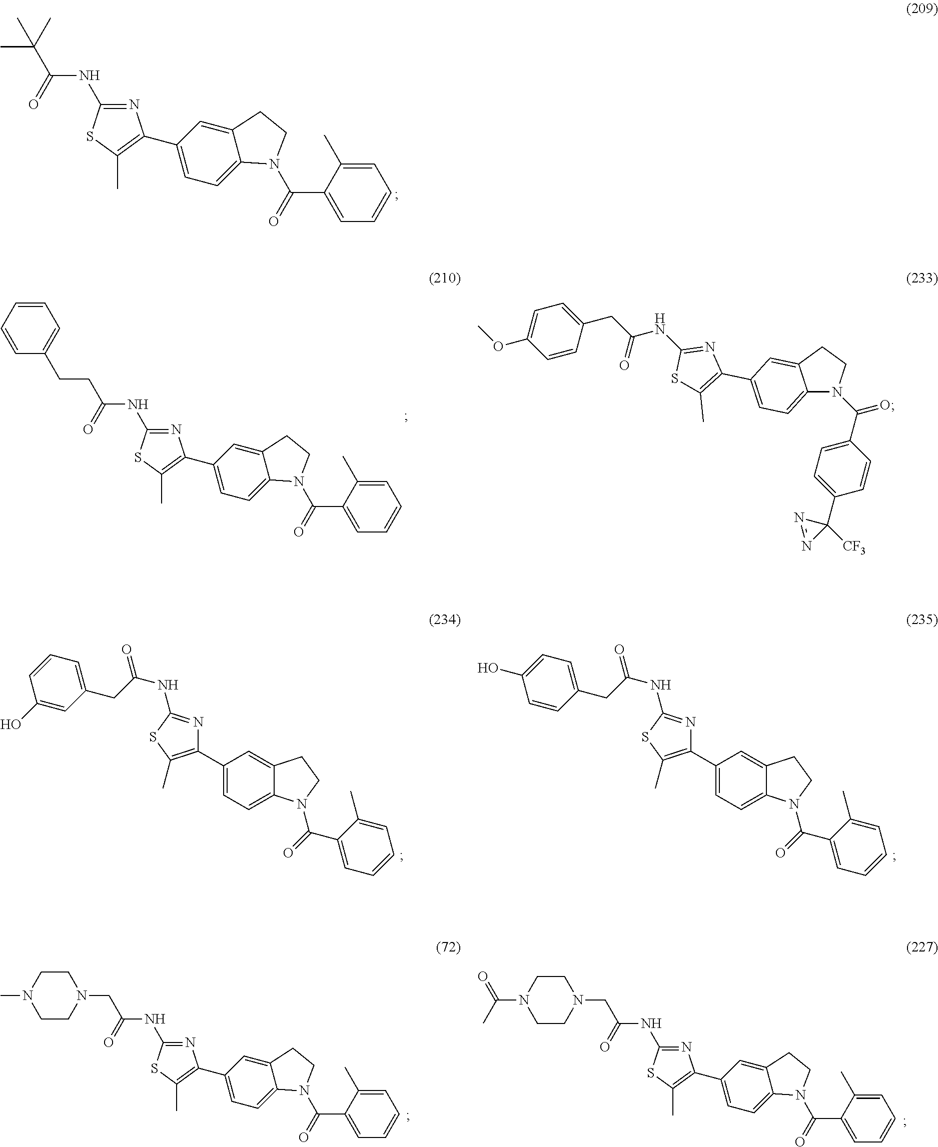

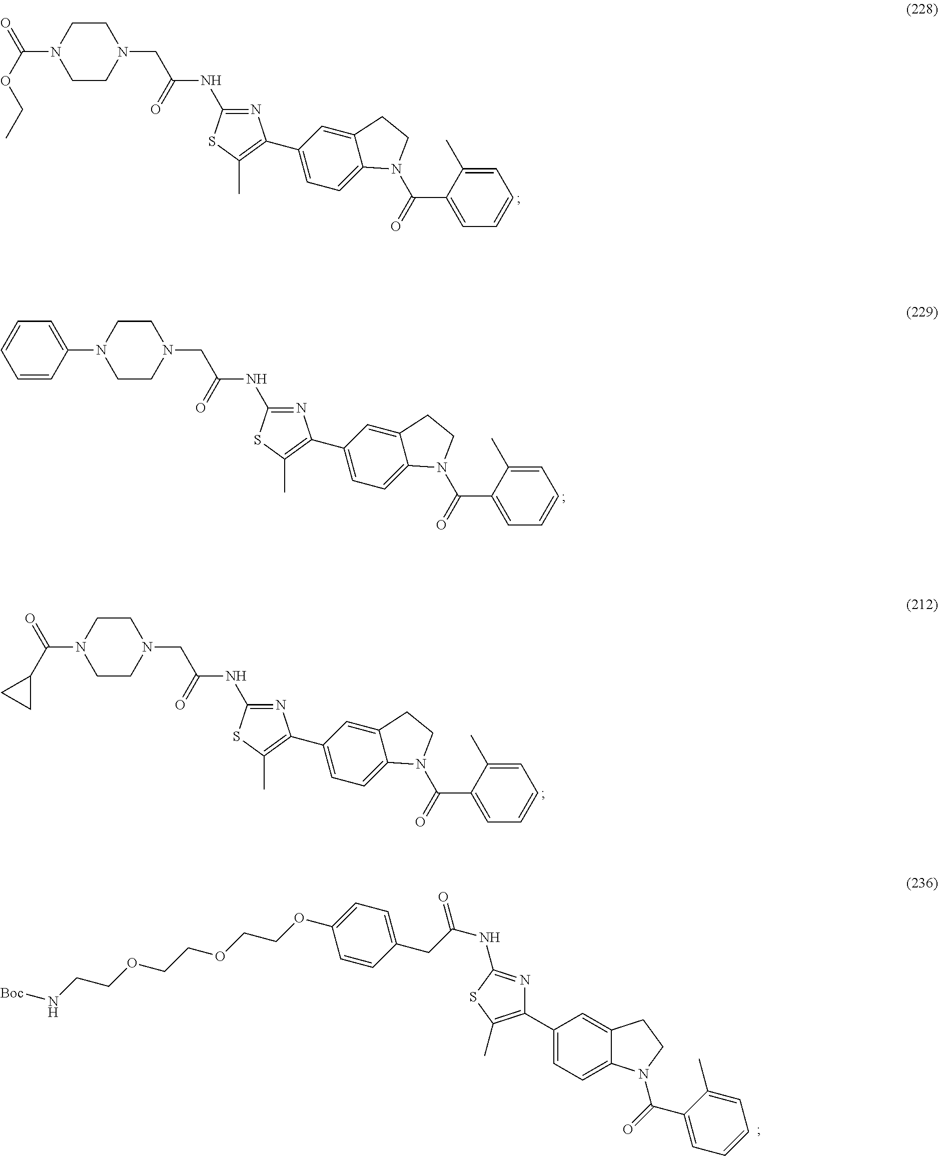 [SMILES: CCOC(=O)N1CCN(CC(=O)Nc2nc(-c3ccc4c(c3)CCN4C(=O)c3ccccc3C)c(C)s2)CC1 ; Cc1ccccc1C(=O)N1CCc2cc(-c3nc(NC(=O)CN4CCN(c5ccccc5)CC4)sc3C)ccc21 ; Cc1ccccc1C(=O)N1CCc2cc(-c3nc(NC(=O)CN4CCN(C(=O)C5CC5)CC4)sc3C)ccc21 ; Cc1ccccc1C(=O)N1CCc2cc(-c3nc(NC(=O)Cc4ccc(OCCOCCOCCNC(=O)OC(C)(C)C)cc4)sc3C)ccc21]

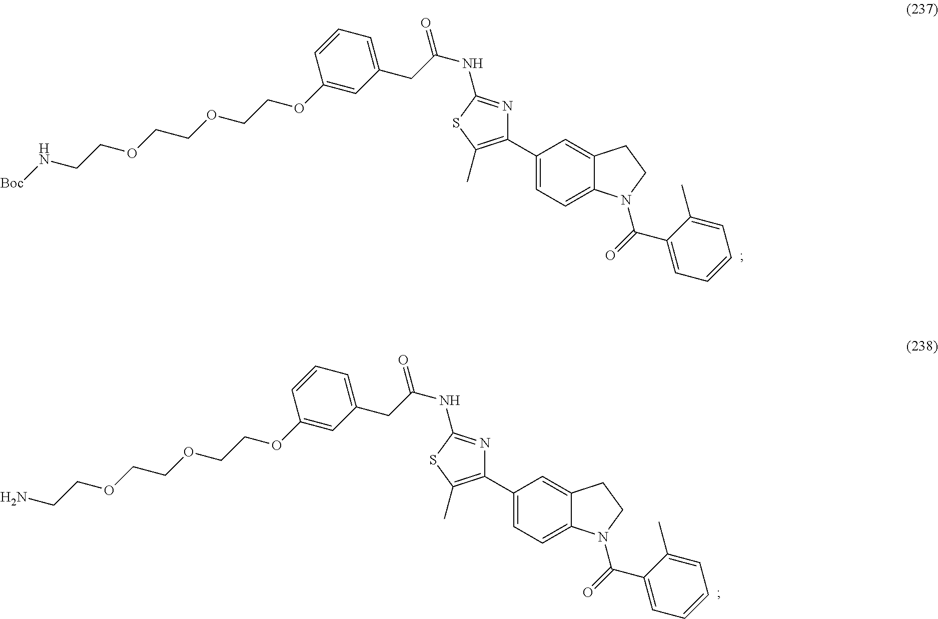 [SMILES: Cc1ccccc1C(=O)N1CCc2cc(-c3nc(NC(=O)Cc4cccc(OCCOCCOCCNC(=O)OC(C)(C)C)c4)sc3C)ccc21 ; Cc1ccccc1C(=O)N1CCc2cc(-c3nc(NC(=O)Cc4cccc(OCCOCCOCCN)c4)sc3C)ccc21]

In other embodiments, the compound is a compound of formula (1a″):

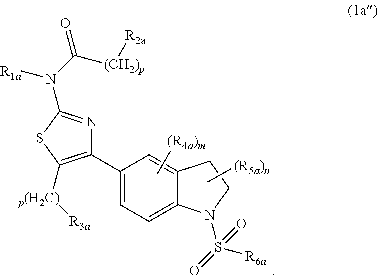

In particular embodiments, the compound of formula (1a″) is selected from the group consisting of:

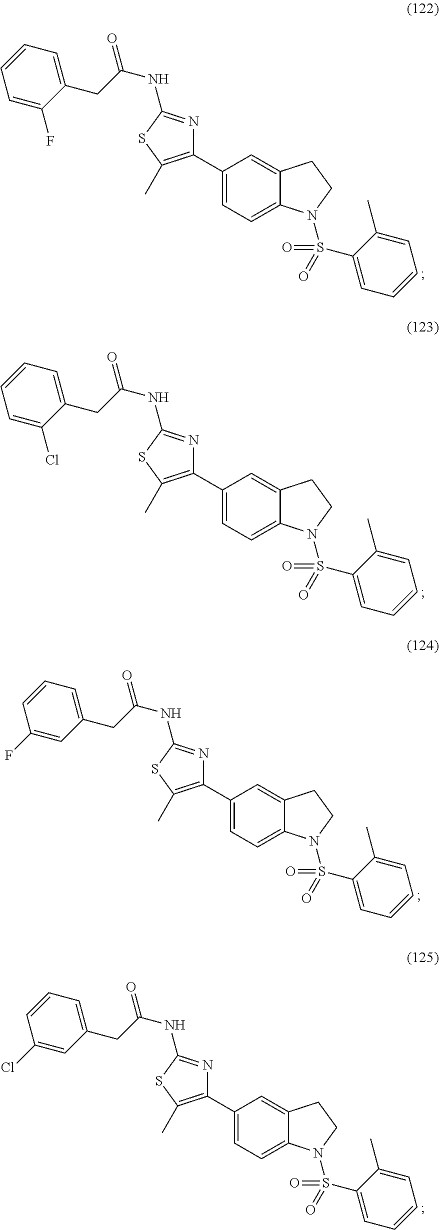 [SMILES: Cc1ccccc1S(=O)(=O)N1CCc2cc(-c3nc(NC(=O)Cc4ccccc4F)sc3C)ccc21 ; Cc1ccccc1S(=O)(=O)N1CCc2cc(-c3nc(NC(=O)Cc4ccccc4Cl)sc3C)ccc21 ; Cc1ccccc1S(=O)(=O)N1CCc2cc(-c3nc(NC(=O)Cc4cccc(F)c4)sc3C)ccc21 ; Cc1ccccc1S(=O)(=O)N1CCc2cc(-c3nc(NC(=O)Cc4cccc(Cl)c4)sc3C)ccc21]

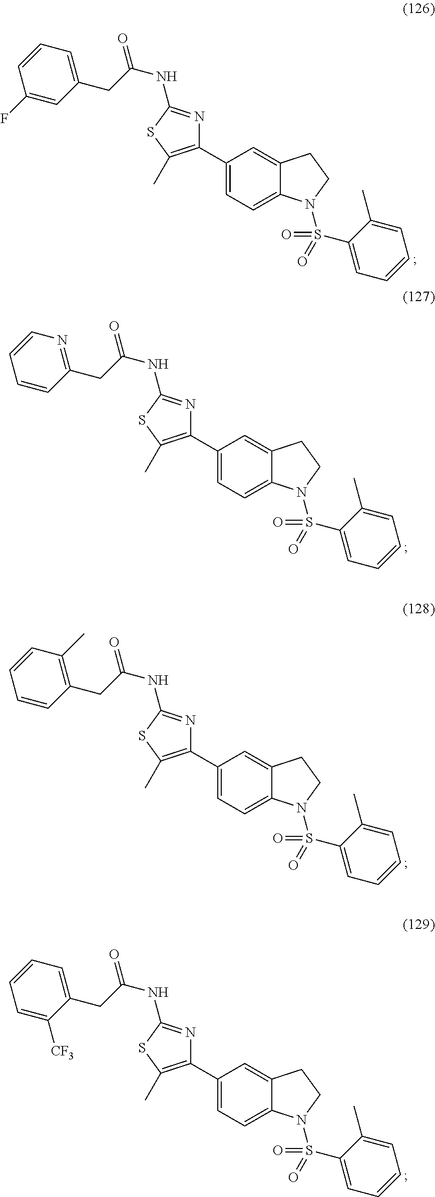 [SMILES: Cc1ccccc1S(=O)(=O)N1CCc2cc(-c3nc(NC(=O)Cc4cccc(F)c4)sc3C)ccc21 ; Cc1ccccc1S(=O)(=O)N1CCc2cc(-c3nc(NC(=O)Cc4ccccn4)sc3C)ccc21 ; Cc1ccccc1CC(=O)Nc1nc(-c2ccc3c(c2)CCN3S(=O)(=O)c2ccccc2C)c(C)s1 ; Cc1ccccc1S(=O)(=O)N1CCc2cc(-c3nc(NC(=O)Cc4ccccc4C(F)(F)F)sc3C)ccc21]

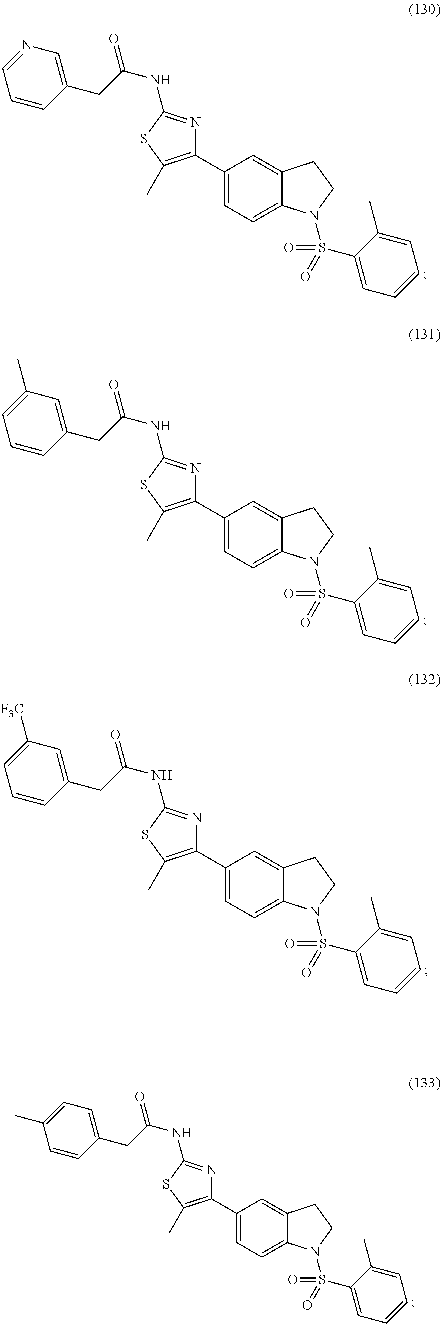 [SMILES: Cc1ccccc1S(=O)(=O)N1CCc2cc(-c3nc(NC(=O)Cc4cccnc4)sc3C)ccc21 ; Cc1cccc(CC(=O)Nc2nc(-c3ccc4c(c3)CCN4S(=O)(=O)c3ccccc3C)c(C)s2)c1 ; Cc1ccccc1S(=O)(=O)N1CCc2cc(-c3nc(NC(=O)Cc4cccc(C(F)(F)F)c4)sc3C)ccc21 ; Cc1ccc(CC(=O)Nc2nc(-c3ccc4c(c3)CCN4S(=O)(=O)c3ccccc3C)c(C)s2)cc1]

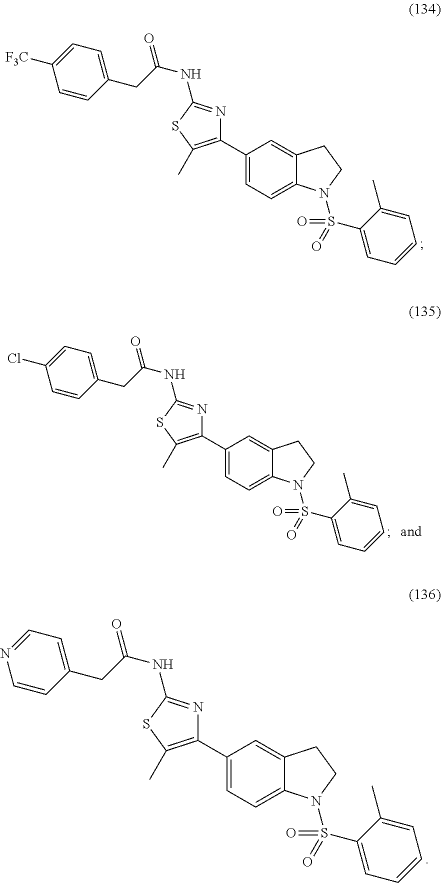 [SMILES: Cc1ccccc1S(=O)(=O)N1CCc2cc(-c3nc(NC(=O)Cc4ccc(C(F)(F)F)cc4)sc3C)ccc21 ; Cc1ccccc1S(=O)(=O)N1CCc2cc(-c3nc(NC(=O)Cc4ccc(Cl)cc4)sc3C)ccc21 ; Cc1ccccc1S(=O)(=O)N1CCc2cc(-c3nc(NC(=O)Cc4ccncc4)sc3C)ccc21]

In yet other embodiments, the compound is a compound of formula (1b″):

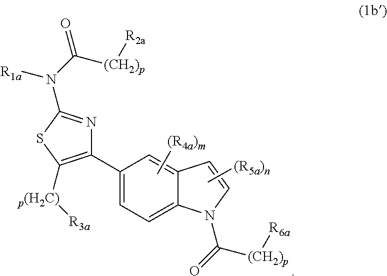

In particular embodiments, the compound of formula (1b′) is:

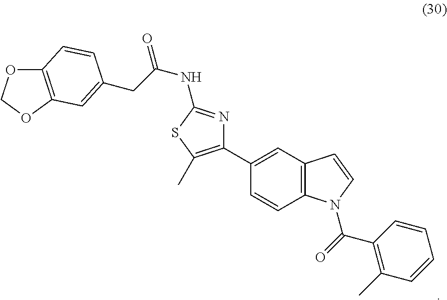 [SMILES: Cc1ccccc1C(=O)n1ccc2cc(-c3nc(NC(=O)Cc4ccc5c(c4)OCO5)sc3C)ccc21]

In yet other embodiments, the compound is selected from the group consisting of:

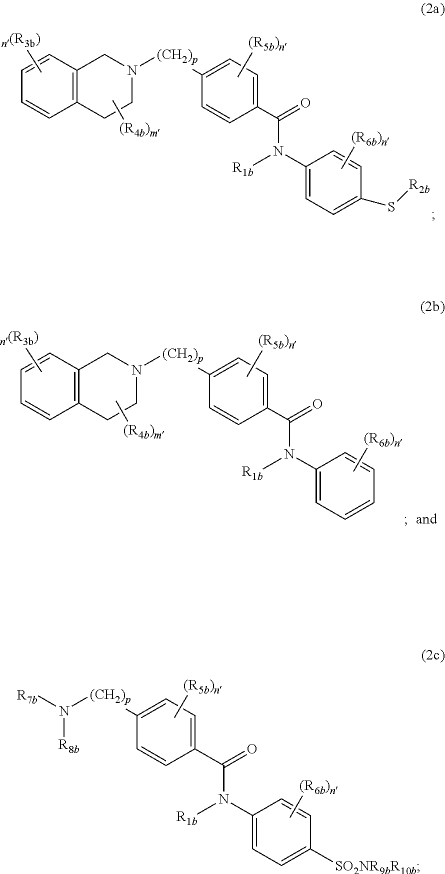

wherein:

m′ is an integer selected from the group consisting of 0, 1, 2, and 3;

n′ is an integer selected from the group consisting of 0, 1, 2, 3, and 4;

each p is independently an integer selected from the group consisting of 0, 1, and 2;

R_{1b} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R_{2b} is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;

each R_{3b}, R_{4b}, R_{5b}, or R_{b6} is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

or for compounds of formula (2b) at least one R_{6b} is selected from the group consisting of H, substituted or unsubstituted cycloheteroalkyl, —(CH₂)ₚ—R_{6b}, and —C(═O)—R_{6b}; p is an integer selected from the group consisting of 0, 1, and 2;

R_{7b} and R_{8b} are each independently selected from the group consisting of substituted or unsubstituted straight-chain or branched alkyl and substituted or unsubstituted cycloheteroalkyl, or R_{7b} and R_{8b} can together form a substituted or unsubstituted heterocyclic ring;

R_{9b} and R_{10b} are each independently selected from the group consisting of substituted or unsubstituted straight-chain or branched alkyl and substituted or unsubstituted cycloheteroalkyl, or R_{9b} and R_{10b} can together form a substituted or unsubstituted heterocyclic ring;

or an enantiomer, diastereomer, racemate or pharmaceutically acceptable salt, prodrug, or solvate thereof;

under the proviso that if the compound is a compound of formula (2a), R_{2b} cannot be —CH₃ or —(O)₂OH.

Accordingly, the presently disclosed compounds are subject to the proviso that they do not include compounds disclosed in Khan, et al., “Identification of Inhibitors of NOD1-Induced Nuclear Factor-KB Activation,” ACS Medicinal Chemistry Letters, 2(10), 780-785 (2011), or U.S. Patent Application Publication No. US2009/0163545 for METHOD FOR ALTERING THE LIFESPAN OF EUKARYOTIC ORGANISMS, to Goldfarb, published Jun. 25, 2009, each of which is incorporated herein in its entirety.

In particular embodiments, the compound is a compound of formula (2b) and the compound is selected from the group consisting of:

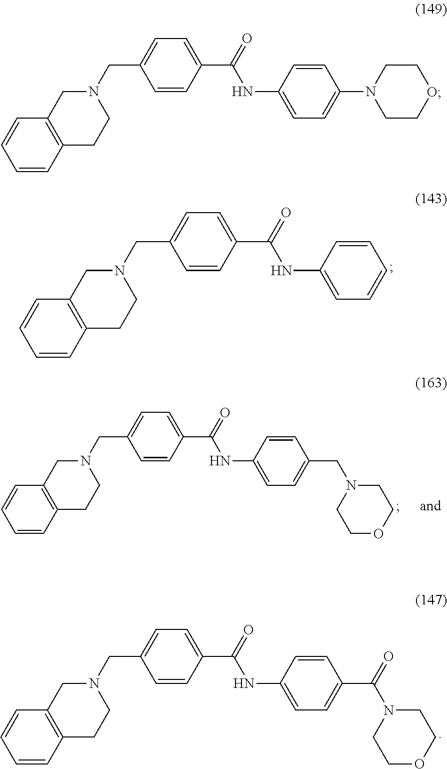 [SMILES: O=C(Nc1ccc(N2CCOCC2)cc1)c1ccc(CN2CCc3ccccc3C2)cc1 ; O=C(Nc1ccccc1)c1ccc(CN2CCc3ccccc3C2)cc1 ; O=C(Nc1ccc(CN2CCOCC2)cc1)c1ccc(CN2CCc3ccccc3C2)cc1 ; O=C(Nc1ccc(C(=O)N2CCOCC2)cc1)c1ccc(CN2CCc3ccccc3C2)cc1]

In particular embodiments, wherein the compound is a compound of formula (2c) and the compound is selected from the group consisting of:

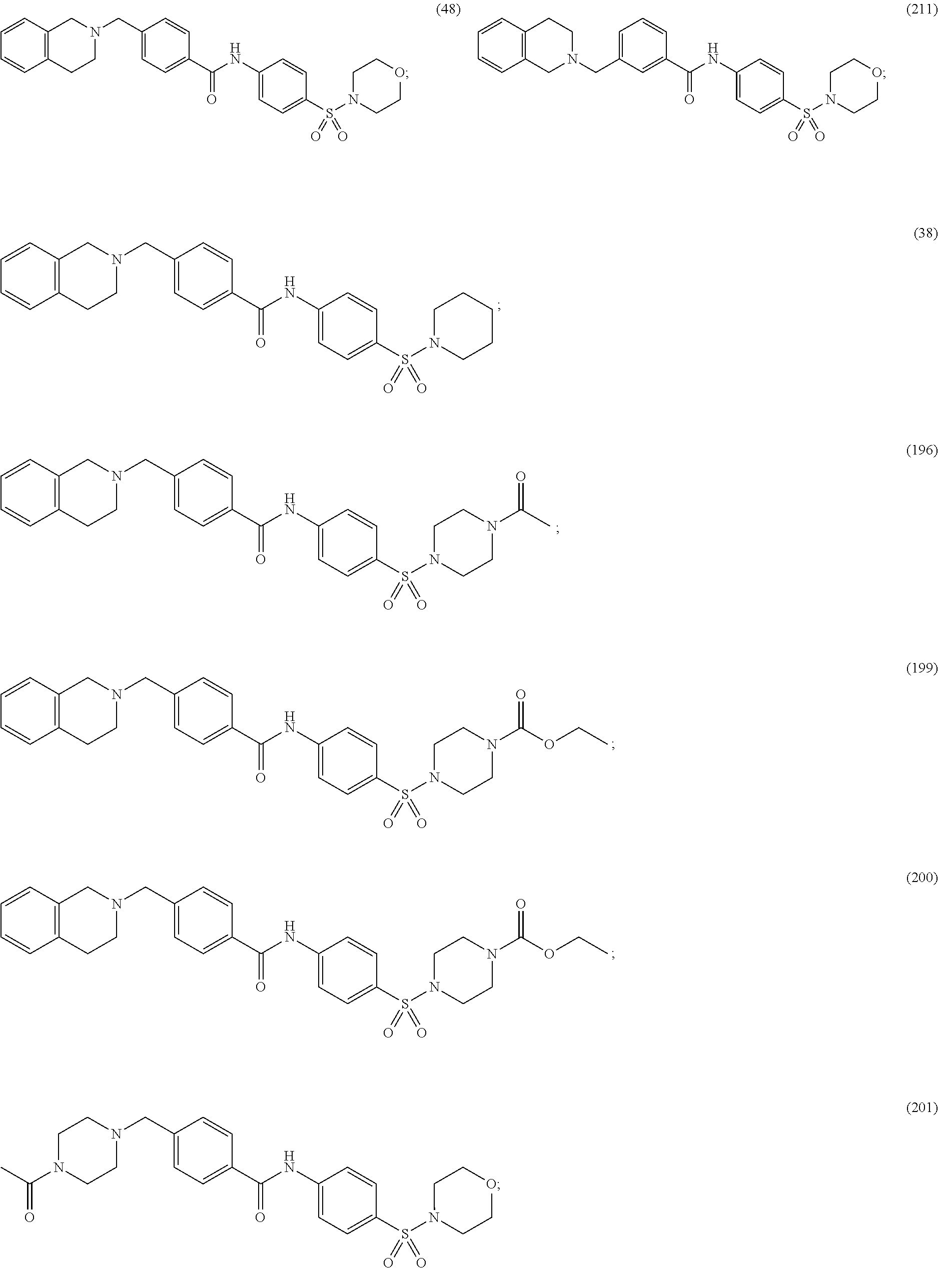

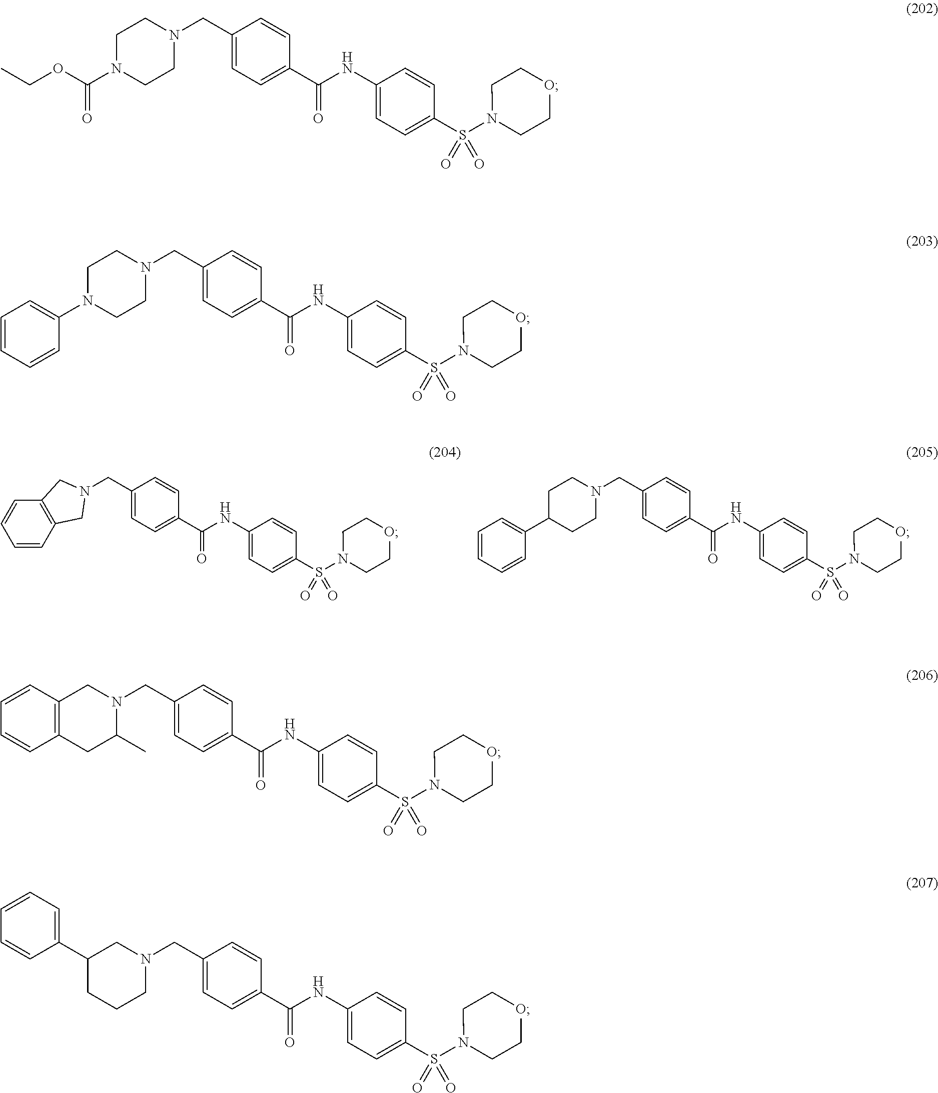 [SMILES: CCOC(=O)N1CCN(Cc2ccc(C(=O)Nc3ccc(S(=O)(=O)N4CCOCC4)cc3)cc2)CC1 ; O=C(Nc1ccc(S(=O)(=O)N2CCOCC2)cc1)c1ccc(CN2CCN(c3ccccc3)CC2)cc1 ; O=C(Nc1ccc(S(=O)(=O)N2CCOCC2)cc1)c1ccc(CN2Cc3ccccc3C2)cc1 ; O=C(Nc1ccc(S(=O)(=O)N2CCOCC2)cc1)c1ccc(CN2CCC(c3ccccc3)CC2)cc1 ; CC1Cc2ccccc2CN1Cc1ccc(C(=O)Nc2ccc(S(=O)(=O)N3CCOCC3)cc2)cc1 ; O=C(Nc1ccc(S(=O)(=O)N2CCOCC2)cc1)c1ccc(CN2CCCC(c3ccccc3)C2)cc1]

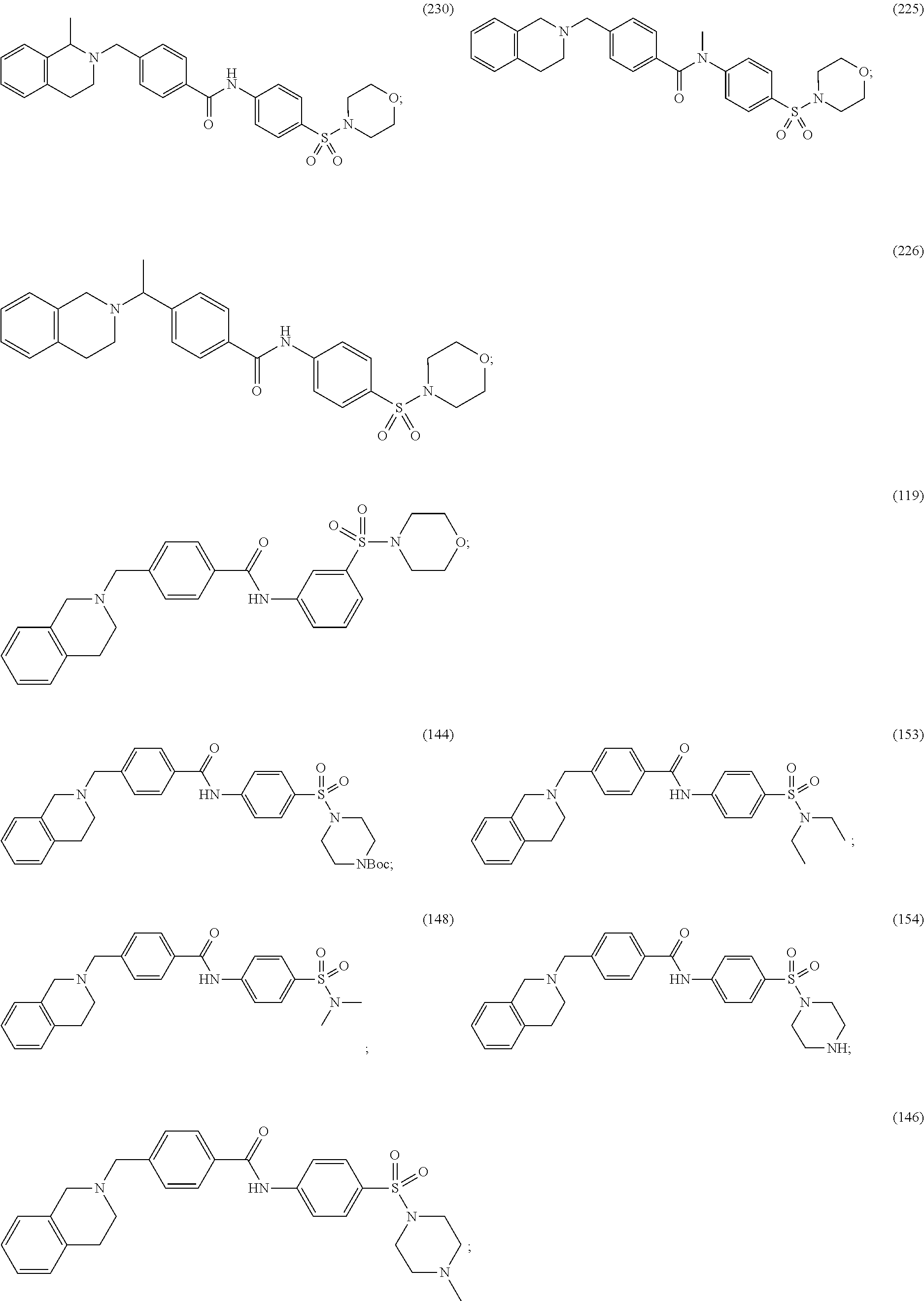

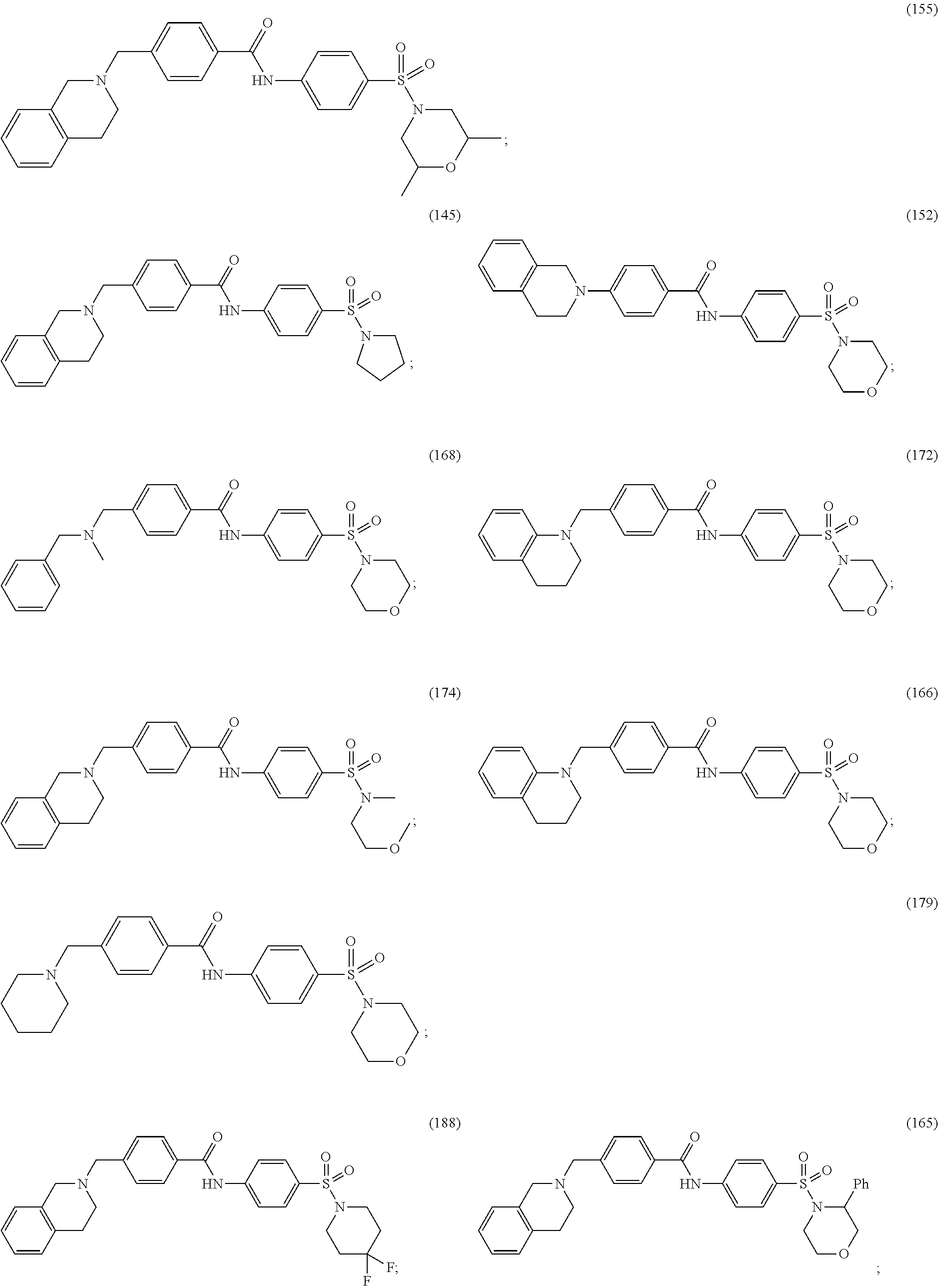

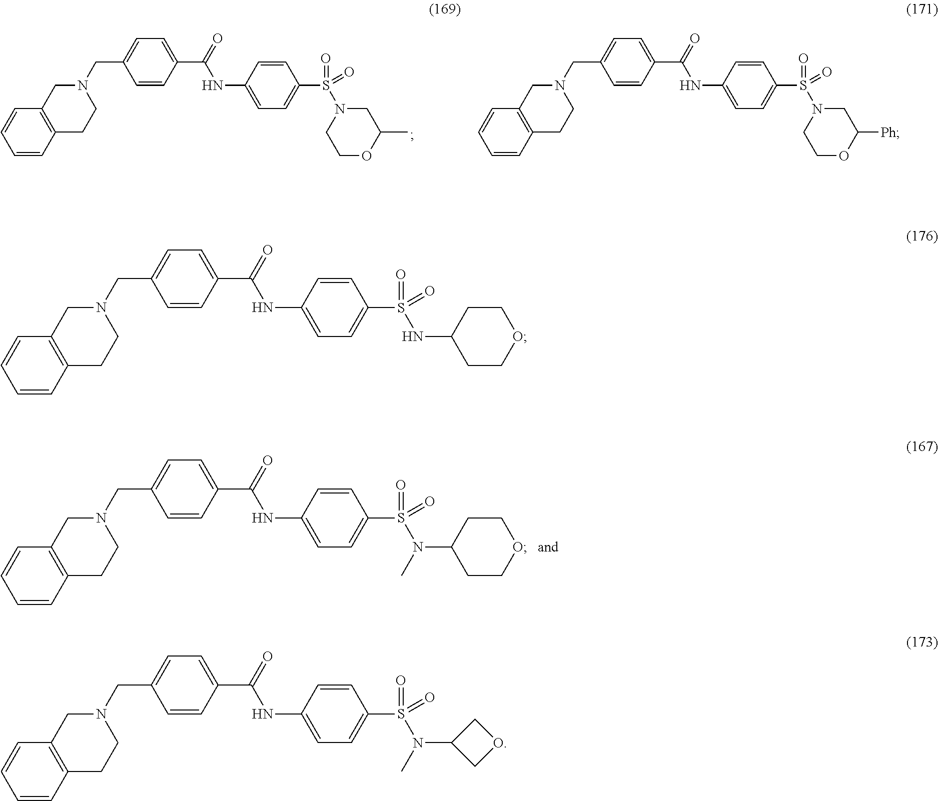 [SMILES: CC1CN(S(=O)(=O)c2ccc(NC(=O)c3ccc(CN4CCc5ccccc5C4)cc3)cc2)CCO1 ; O=C(Nc1ccc(S(=O)(=O)N2CCOC(c3ccccc3)C2)cc1)c1ccc(CN2CCc3ccccc3C2)cc1 ; O=C(Nc1ccc(S(=O)(=O)NC2CCOCC2)cc1)c1ccc(CN2CCc3ccccc3C2)cc1 ; CN(C1CCOCC1)S(=O)(=O)c1ccc(NC(=O)c2ccc(CN3CCc4ccccc4C3)cc2)cc1 ; "and" ; CN(C1COC1)S(=O)(=O)c1ccc(NC(=O)c2ccc(CN3CCc4ccccc4C3)cc2)cc1]

In yet other embodiments, the presently disclosed subject matter provides a compound of formula (3):

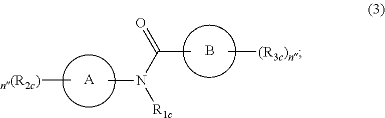

wherein:

each n″ is an integer independently selected from the group consisting of 0, 1, 2, 3, 4, 5, and 6, depending on the maximum available atoms on ring A and ring B;

A is a ring structure selected from the group consisting of:

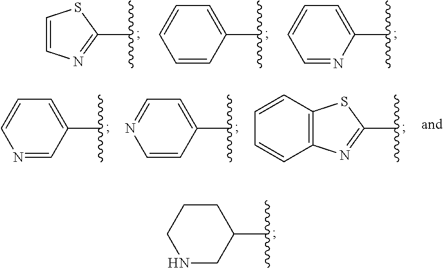

B is —(CH₂)ₙ— or a ring structure selected from the group consisting of:

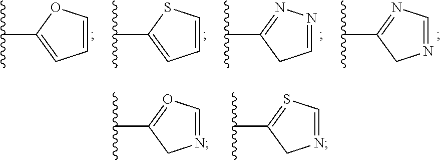

wherein the ring structure A and ring structure B are connected via an amide linkage represented by —NR_{1c}C(═O)—;

R_{1c} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;

R_{2c} and R_{3c} are each independently selected from the group consisting of substituted or unsubstituted straight-chain or branched alkyl and —(CH₂)ₚ—Cy,

wherein p is an integer selected from the group consisting of 0, 1, and 2; and Cy is selected from the group consisting of substituted or unsubstituted cycloheteroalkyl, substituted or unsubstituted aryl, and substituted or unsubstituted heteroaryl;

or an enantiomer, diastereomer, racemate or pharmaceutically acceptable salt, prodrug, or solvate thereof.

In particular embodiments, the compound of formula (3) is selected from the group consisting of:

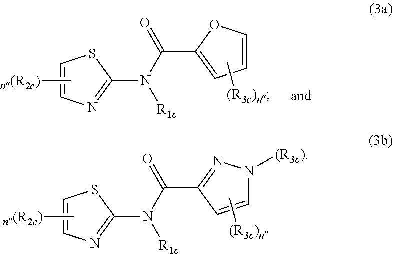

In certain embodiments, the compound is a compound of Formula (3a) and the compound is selected from the group consisting of:

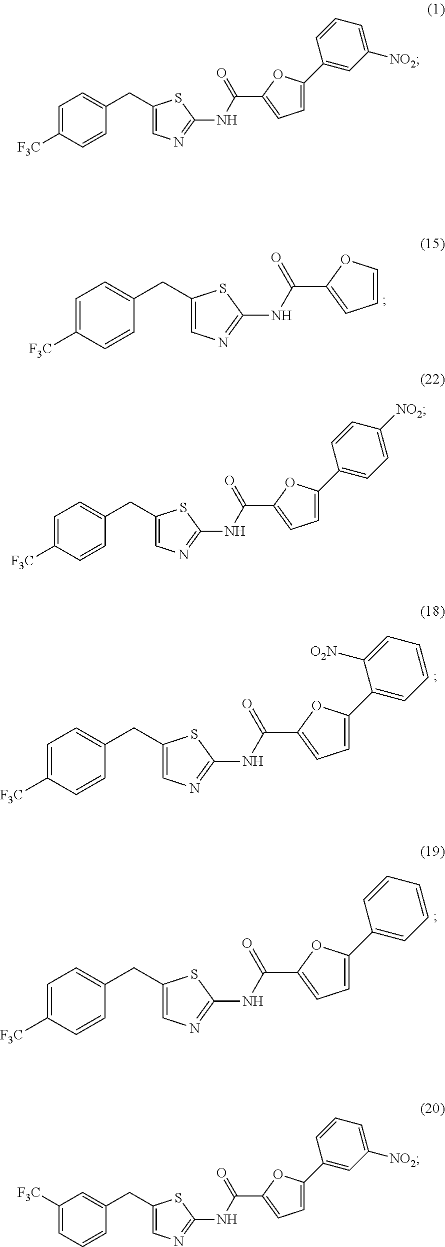 [SMILES: O=C(Nc1ncc(Cc2ccc(C(F)(F)F)cc2)s1)c1ccc(-c2cccc([N+](=O)[O-])c2)o1 ; O=C(Nc1ncc(Cc2ccc(C(F)(F)F)cc2)s1)c1ccco1 ; O=C(Nc1ncc(Cc2ccc(C(F)(F)F)cc2)s1)c1ccc(-c2ccc([N+](=O)[O-])cc2)o1 ; O=C(Nc1ncc(Cc2ccc(C(F)(F)F)cc2)s1)c1ccc(-c2ccccc2[N+](=O)[O-])o1 ; O=C(Nc1ncc(Cc2ccc(C(F)(F)F)cc2)s1)c1ccc(-c2ccccc2)o1 ; O=C(Nc1ncc(Cc2cccc(C(F)(F)F)c2)s1)c1ccc(-c2cccc([N+](=O)[O-])c2)o1]

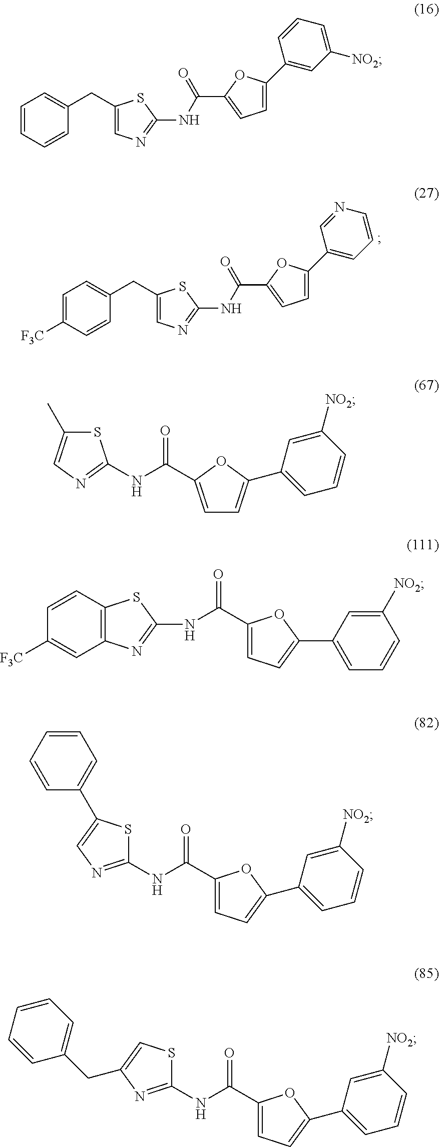 [SMILES: O=C(Nc1ncc(Cc2ccccc2)s1)c1ccc(-c2cccc([N+](=O)[O-])c2)o1 ; O=C(Nc1ncc(Cc2ccc(C(F)(F)F)cc2)s1)c1ccc(-c2cccnc2)o1 ; Cc1cnc(NC(=O)c2ccc(-c3cccc([N+](=O)[O-])c3)o2)s1 ; O=C(Nc1nc2cc(C(F)(F)F)ccc2s1)c1ccc(-c2cccc([N+](=O)[O-])c2)o1 ; O=C(Nc1ncc(-c2ccccc2)s1)c1ccc(-c2cccc([N+](=O)[O-])c2)o1 ; O=C(Nc1nc(Cc2ccccc2)cs1)c1ccc(-c2cccc([N+](=O)[O-])c2)o1]

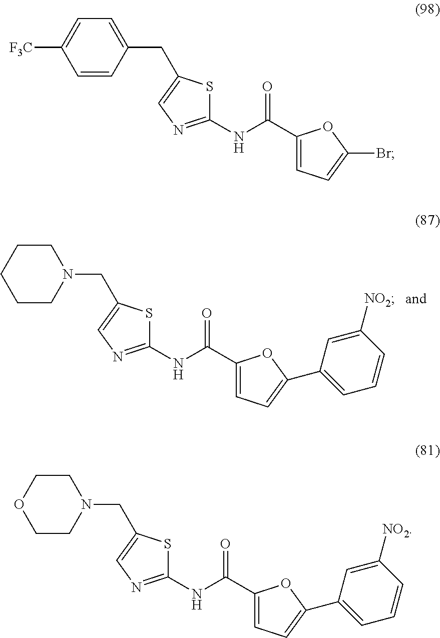 [SMILES: O=C(Nc1ncc(Cc2ccc(C(F)(F)F)cc2)s1)c1ccc(Br)o1 ; O=C(Nc1ncc(CN2CCCCC2)s1)c1ccc(-c2cccc([N+](=O)[O-])c2)o1 ; "and" ; O=C(Nc1ncc(CN2CCOCC2)s1)c1ccc(-c2cccc([N+](=O)[O-])c2)o1]

In yet other certain embodiments, the compound is a compound of Formula (3b) and the compound is selected from the group consisting of:

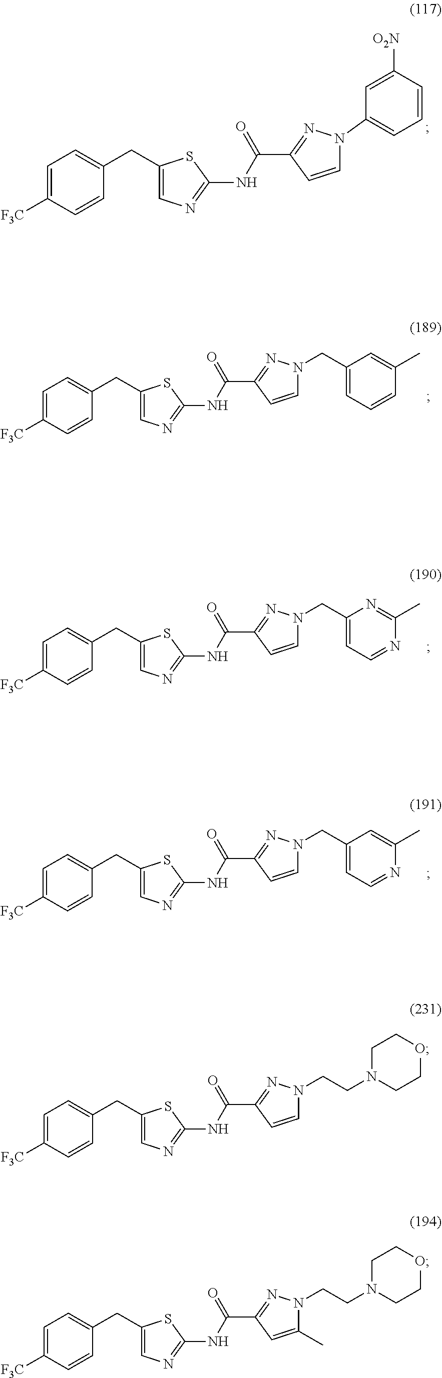 [SMILES: O=C(Nc1ncc(Cc2ccc(C(F)(F)F)cc2)s1)c1ccn(-c2cccc([N+](=O)[O-])c2)n1 ; Cc1cccc(Cn2ccc(C(=O)Nc3ncc(Cc4ccc(C(F)(F)F)cc4)s3)n2)c1 ; Cc1nccc(Cn2ccc(C(=O)Nc3ncc(Cc4ccc(C(F)(F)F)cc4)s3)n2)n1 ; Cc1cc(Cn2ccc(C(=O)Nc3ncc(Cc4ccc(C(F)(F)F)cc4)s3)n2)ccn1 ; O=C(Nc1ncc(Cc2ccc(C(F)(F)F)cc2)s1)c1ccn(CCN2CCOCC2)n1 ; Cc1cc(C(=O)Nc2ncc(Cc3ccc(C(F)(F)F)cc3)s2)nn1CCN1CCOCC1]

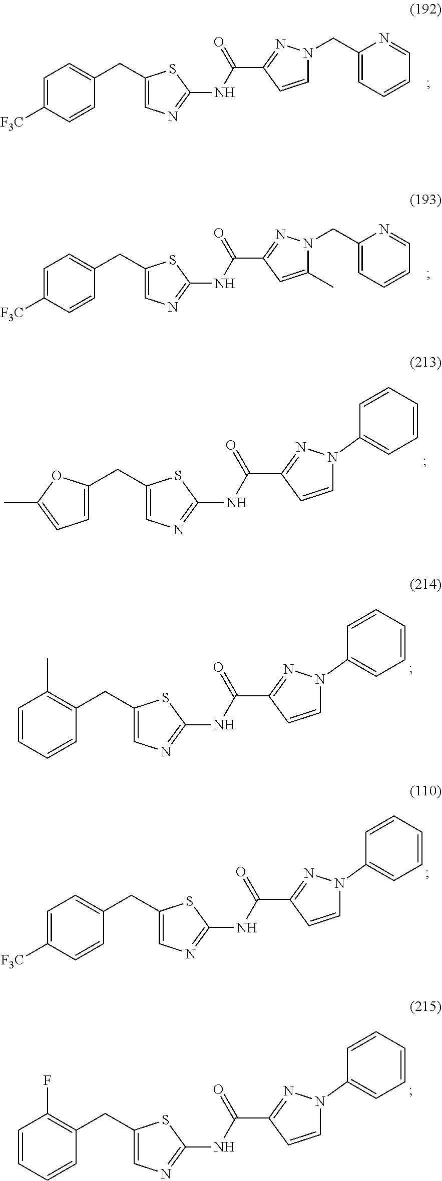 [SMILES: O=C(Nc1ncc(Cc2ccc(C(F)(F)F)cc2)s1)c1ccn(Cc2ccccn2)n1 ; Cc1cc(C(=O)Nc2ncc(Cc3ccc(C(F)(F)F)cc3)s2)nn1Cc1ccccn1 ; Cc1ccc(Cc2cnc(NC(=O)c3ccn(-c4ccccc4)n3)s2)o1 ; Cc1ccccc1Cc1cnc(NC(=O)c2ccn(-c3ccccc3)n2)s1 ; O=C(Nc1ncc(Cc2ccc(C(F)(F)F)cc2)s1)c1ccn(-c2ccccc2)n1 ; O=C(Nc1ncc(Cc2ccccc2F)s1)c1ccn(-c2ccccc2)n1]

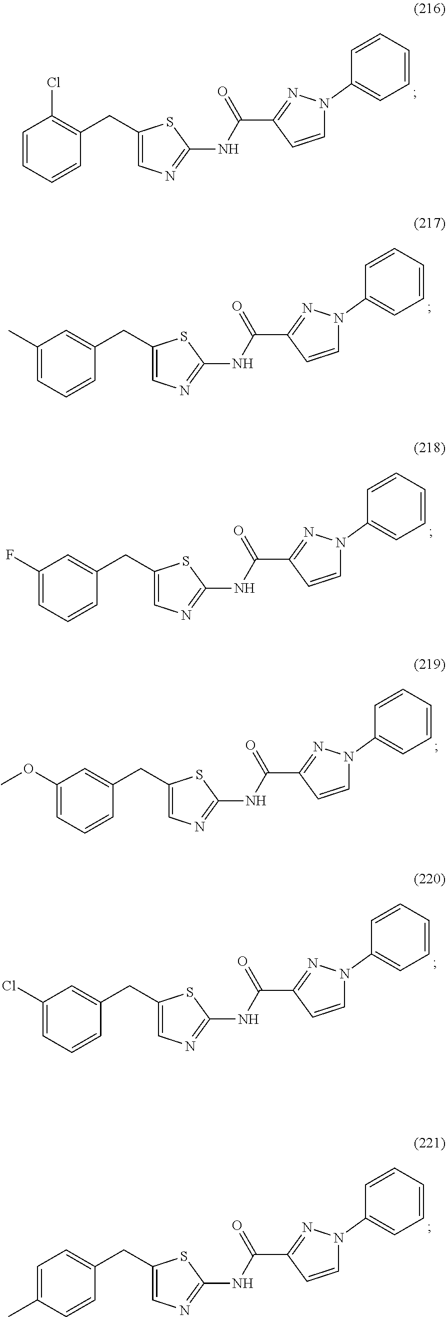 [SMILES: O=C(Nc1ncc(Cc2ccccc2Cl)s1)c1ccn(-c2ccccc2)n1 ; Cc1cccc(Cc2cnc(NC(=O)c3ccn(-c4ccccc4)n3)s2)c1 ; O=C(Nc1ncc(Cc2cccc(F)c2)s1)c1ccn(-c2ccccc2)n1 ; COc1cccc(Cc2cnc(NC(=O)c3ccn(-c4ccccc4)n3)s2)c1 ; O=C(Nc1ncc(Cc2cccc(Cl)c2)s1)c1ccn(-c2ccccc2)n1 ; Cc1ccc(Cc2cnc(NC(=O)c3ccn(-c4ccccc4)n3)s2)cc1]

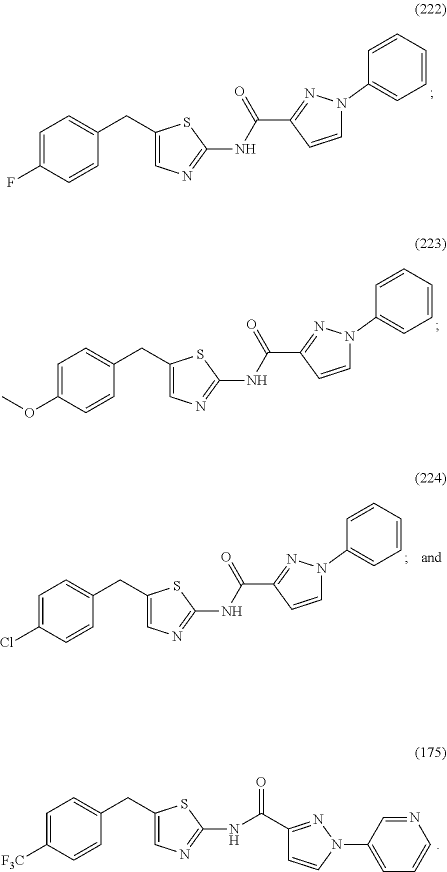 [SMILES: O=C(Nc1ncc(Cc2ccc(F)cc2)s1)c1ccn(-c2ccccc2)n1 ; COc1ccc(Cc2cnc(NC(=O)c3ccn(-c4ccccc4)n3)s2)cc1 ; O=C(Nc1ncc(Cc2ccc(Cl)cc2)s1)c1ccn(-c2ccccc2)n1 ; O=C(Nc1ncc(Cc2ccc(C(F)(F)F)cc2)s1)c1ccn(-c2cccnc2)n1]

In certain embodiments of compounds of formula (3),
A is thiazolyl;
B is selected from the group consisting of phenyl, pyridinyl, imidazolyl, oxazolyl, thiophenyl, thiazolyl, and —(CH₂)ₙ—; and
the compound is selected from the group consisting of:

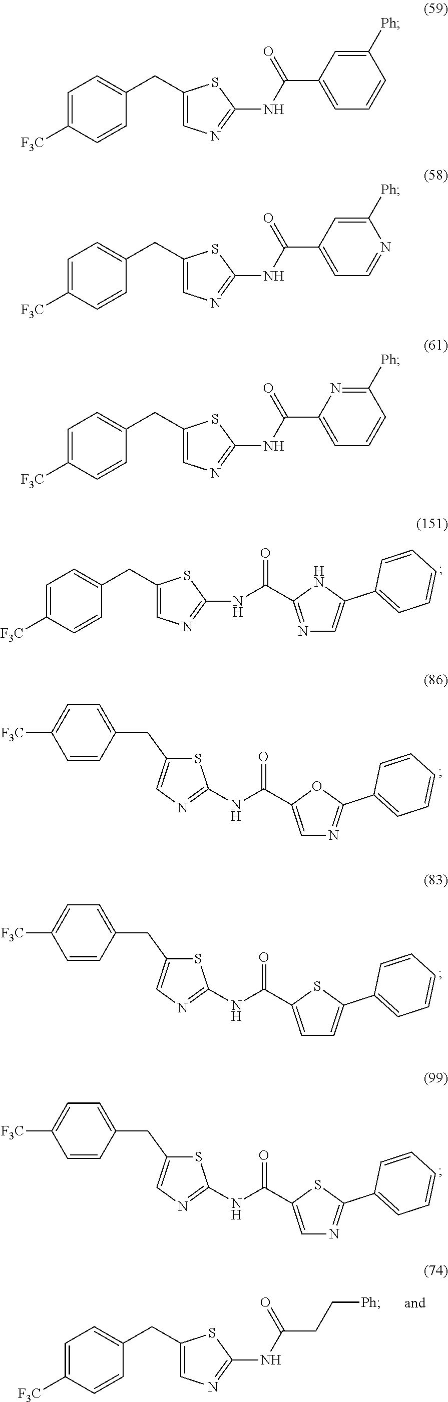

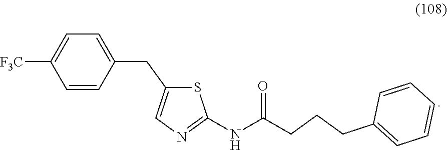 [SMILES: O=C(CCCc1ccccc1)Nc1ncc(Cc2ccc(C(F)(F)F)cc2)s1]

In certain other embodiments of compounds of formula (3),
A is selected from the group consisting of phenyl, pyridinyl, and piperidinyl;
B is furanyl; and
the compound is selected from the group consisting of:

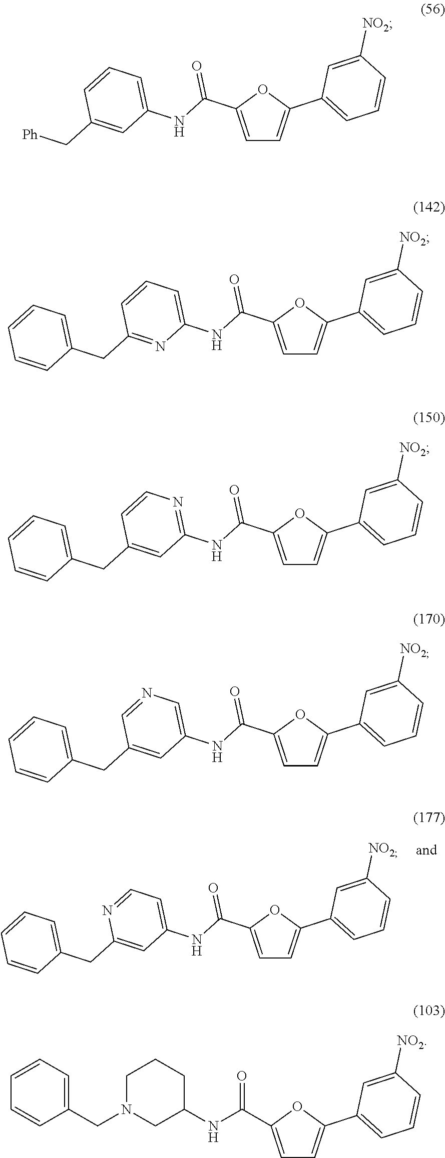 [SMILES: O=C(Nc1cccc(Cc2ccccc2)c1)c1ccc(-c2cccc([N+](=O)[O-])c2)o1 ; O=C(Nc1cccc(Cc2ccccc2)n1)c1ccc(-c2cccc([N+](=O)[O-])c2)o1 ; O=C(Nc1cc(Cc2ccccc2)ccn1)c1ccc(-c2cccc([N+](=O)[O-])c2)o1 ; O=C(Nc1cncc(Cc2ccccc2)c1)c1ccc(-c2cccc([N+](=O)[O-])c2)o1 ; O=C(Nc1ccnc(Cc2ccccc2)c1)c1ccc(-c2cccc([N+](=O)[O-])c2)o1 ; O=C(NC1CCCN(Cc2ccccc2)C1)c1ccc(-c2cccc([N+](=O)[O-])c2)o1]

In yet other certain embodiments of compounds of formula (3):
A is phenyl;
B is selected from the group consisting of pyridinyl, pyrimidinyl, pyrrolidinyl, piperidinyl, and pyrazolyl; and
the compound is selected from the group consisting of:

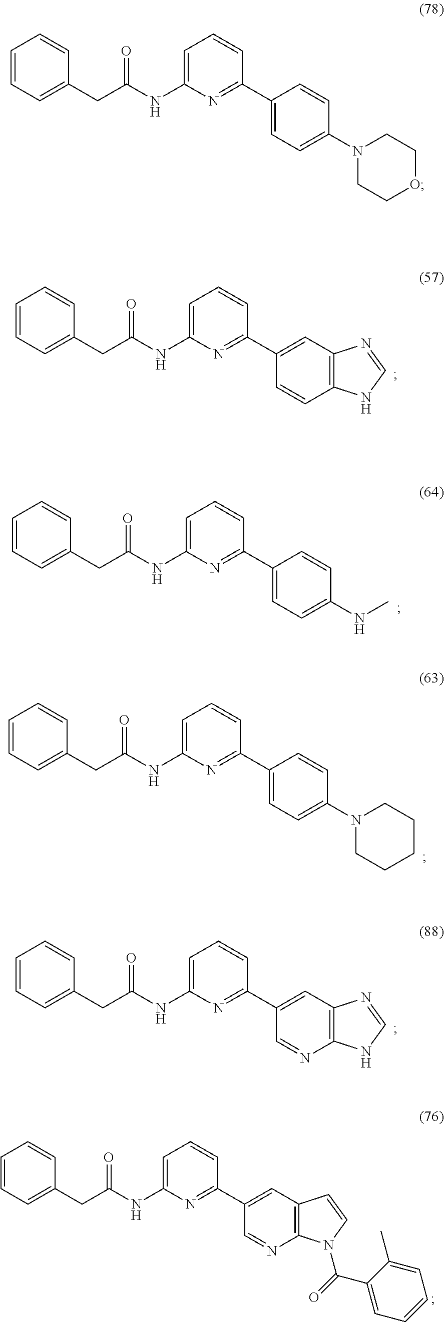 [SMILES: O=C(Cc1ccccc1)Nc1cccc(-c2ccc(N3CCOCC3)cc2)n1 ; O=C(Cc1ccccc1)Nc1cccc(-c2ccc3[nH]cnc3c2)n1 ; CNc1ccc(-c2cccc(NC(=O)Cc3ccccc3)n2)cc1 ; O=C(Cc1ccccc1)Nc1cccc(-c2ccc(N3CCCCC3)cc2)n1 ; O=C(Cc1ccccc1)Nc1cccc(-c2cnc3[nH]cnc3c2)n1 ; Cc1ccccc1C(=O)n1ccc2cc(-c3cccc(NC(=O)Cc4ccccc4)n3)cnc21]

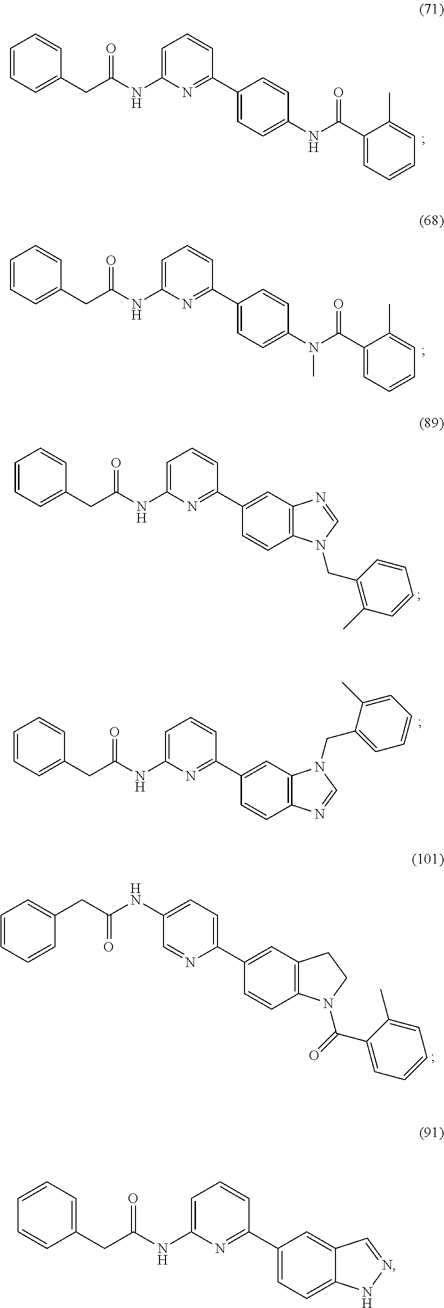

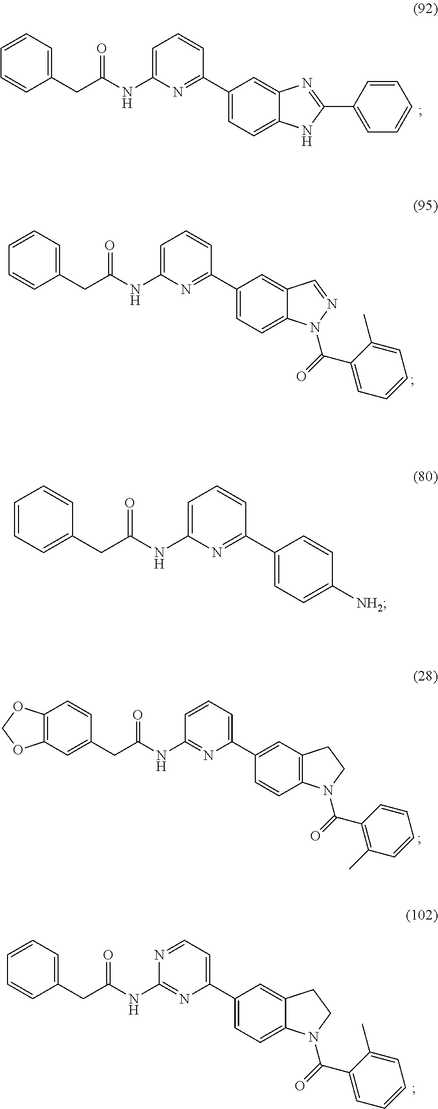 [SMILES: O=C(Cc1ccccc1)Nc1cccc(-c2ccc3[nH]c(-c4ccccc4)nc3c2)n1 ; Cc1ccccc1C(=O)n1ncc2cc(-c3cccc(NC(=O)Cc4ccccc4)n3)ccc21 ; Nc1ccc(-c2cccc(NC(=O)Cc3ccccc3)n2)cc1 ; Cc1ccccc1C(=O)N1CCc2cc(-c3cccc(NC(=O)Cc4ccc5c(c4)OCO5)n3)ccc21 ; Cc1ccccc1C(=O)N1CCc2cc(-c3ccnc(NC(=O)Cc4ccccc4)n3)ccc21]

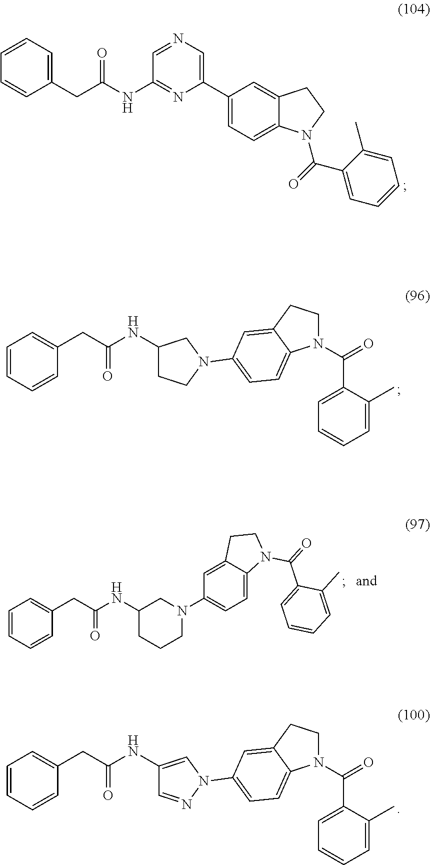 [SMILES: Cc1ccccc1C(=O)N1CCc2cc(-c3cncc(NC(=O)Cc4ccccc4)n3)ccc21 ; Cc1ccccc1C(=O)N1CCc2cc(N3CCC(NC(=O)Cc4ccccc4)C3)ccc21 ; Cc1ccccc1C(=O)N1CCc2cc(N3CCCC(NC(=O)Cc4ccccc4)C3)ccc21 ; Cc1ccccc1C(=O)N1CCc2cc(-n3cc(NC(=O)Cc4ccccc4)cn3)ccc21]

In yet other certain embodiments of compounds of formula (3),
A is phenyl;
B forms an indolinyl ring structure with the amide linkage; and
the compound is selected from the group consisting of:

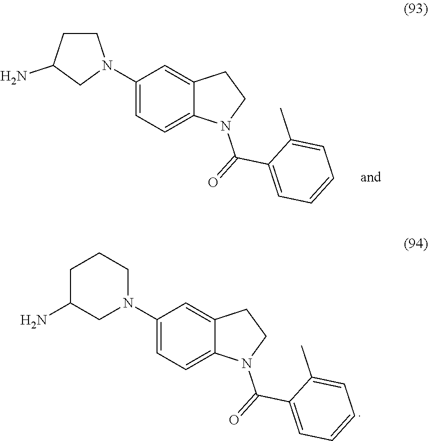 [SMILES: Cc1ccccc1C(=O)N1CCc2cc(N3CCC(N)C3)ccc21 ; Cc1ccccc1C(=O)N1CCc2cc(N3CCCC(N)C3)ccc21]

In particular embodiments of compounds of formula (3), A and B are both phenyl and compound of formula (3) has the following structure:

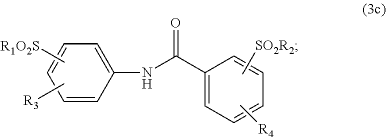

wherein:

—SO₂R₁ and —SO₂R₂ can each be present or absent and, if present, R₁ and R₂ can each independently be substituted or unsubstituted heterocycloalkyl;

R₃ is selected from the group consisting of H, alkyl, O-alkyl and halogen;

R₄ is selected from the group consisting of H, alkyl, O-alkyl and halogen;

or an enantiomer, diastereomer, racemate or pharmaceutically acceptable salt, prodrug, or solvate thereof.

In certain embodiments, the compound of formula (3c) is selected from the group consisting of:

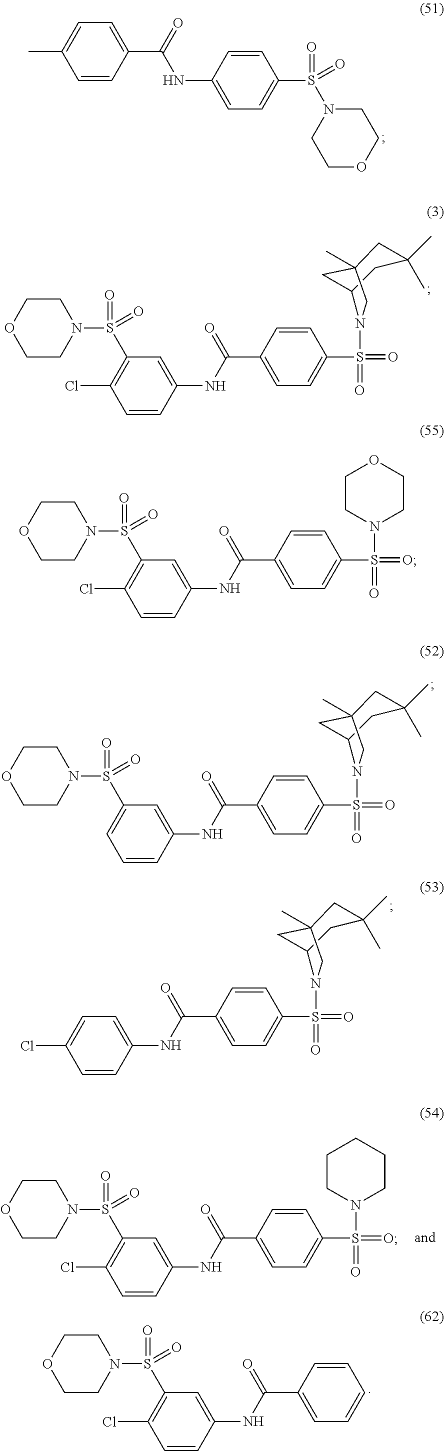

Additional compounds of the presently disclosed subject matter include, but are not limited to the following:

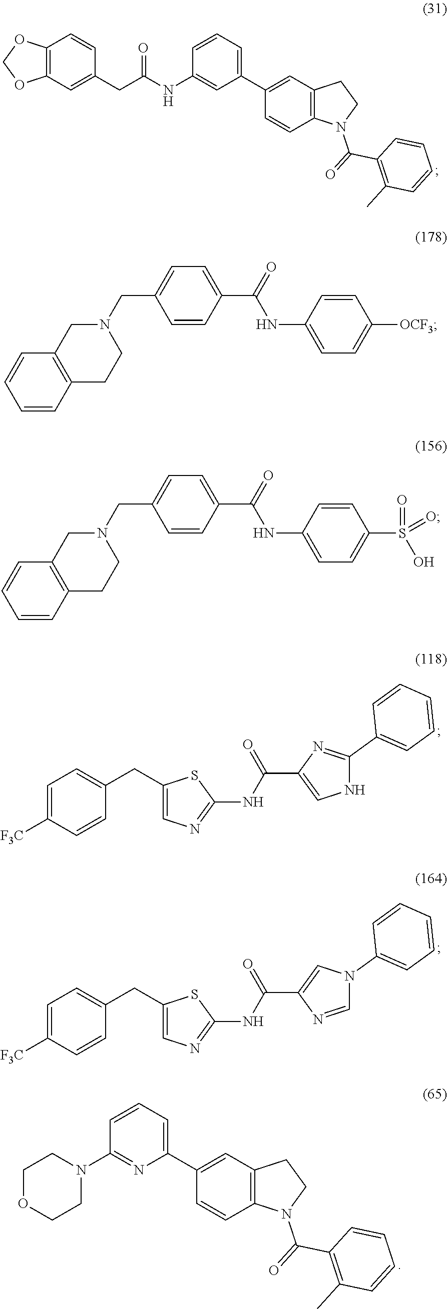 [SMILES: Cc1ccccc1C(=O)N1CCc2cc(-c3cccc(NC(=O)Cc4ccc5c(c4)OCO5)c3)ccc21 ; O=C(Nc1ccc(OC(F)(F)F)cc1)c1ccc(CN2CCc3ccccc3C2)cc1 ; O=C(Nc1ccc(S(=O)(=O)O)cc1)c1ccc(CN2CCc3ccccc3C2)cc1 ; O=C(Nc1ncc(Cc2ccc(C(F)(F)F)cc2)s1)c1c[nH]c(-c2ccccc2)n1 ; O=C(Nc1ncc(Cc2ccc(C(F)(F)F)cc2)s1)c1cn(-c2ccccc2)cn1 ; Cc1ccccc1C(=O)N1CCc2cc(-c3cccc(N4CCOCC4)n3)ccc21]

While the following terms in relation to compounds of formula (1-3) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms “substituted,” whether preceded by the term “optionally” or not, and “substituent,” as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “hydrocarbon,” as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, 0-CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopropyl, cyclobutyl, cyclopentyl, cyclohexyl, cycloheptyl, bicycle[2.2.1]heptyl, cyclopentenyl, and cyclohexenyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, diazabicyclo[2.2.1]hept-2-yl, benzofuranyl, benzothienyl, benzodioxolyl, quinolinyl, thiadiazolyl, e.g., 1,2,3-thiadiazolyl, 2,3-diydrobenzofuranyl, tetrahydropyranyl, imidazo[1,2-a]pyridinyl, thiazolidinyl, indanyl, pyridazinyl, furanyl, pyrimidinyl, triazolyl, pyridinyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, oxadiazolyl, e.g., 1,2,4-oxadiazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, indazolyl, 1-isoquinolyl, 5-isoquinolyl, 2-quinoxalinyl, 5-quinoxalinyl, 3-quinolyl, 6-quinolyl, tetrazolyl, benzo[d]isoxazolyl, and benzo[d][1,3]dioxolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(l-naphthyloxy)propyl, and the like). The term “haloaryl,” however, as used herein, is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

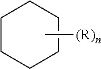

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

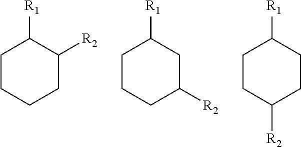

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure. The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond. Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+l), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms “alkylthioether” and “thioalkoxyl” refer to a saturated (alkyl-S—) or unsaturated (alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “thiohydroxyl” or “thiol,” as used herein, refers to a group of the formula —SH.

The term “ureido” refers to a urea group of the formula —NH—CO—NH₂.

Unless otherwise explicitly defined, a “substituent group,” as used herein, includes a functional group selected from one or more of the following moieties, which are defined herein:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “lower substituent” or “lower substituent group,” as used herein means a group selected from all of the substituents described hereinabove for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

Throughout the specification and claims, a given chemical formula or name shall encompass all tautomers, congeners, and optical- and stereoisomers, as well as racemic mixtures where such isomers and mixtures exist.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

B. Pharmaceutical Compositions of the Presently Disclosed Compounds

In some embodiments, the presently disclosed subject matter provides a pharmaceutical composition comprising an Nrf2 inhibitor and a pharmaceutically acceptable carrier, for example, pharmaceutical composition including one or more Nrf2 inhibitors, alone or in combination with one or more additional therapeutic agents in admixture with a pharmaceutically acceptable excipient. The term “pharmaceutically-acceptable excipient” as used herein means one or more compatible solid or liquid filler, diluents or encapsulating substances that are suitable for administration into a subject. One of skill in the art will recognize that the pharmaceutical compositions include the pharmaceutically acceptable salts of the compounds.

In some embodiments, the pharmaceutical composition further comprises one or more chemotherapeutic drugs. In some embodiments, the chemotherapeutic drug is selected from the group consisting of a topoisomerase inhibitor, alkylating agent, antimetabolite, anthracycline, and plant alkoid. In other embodiments, the chemotherapeutic drug is selected from the group consisting of etoposide, cisplatin, paclitaxel, gemcitabine, and carboplatin.

The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids, such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

One of ordinary skill in the art would appreciate that certain substituent groups can be added to the presently disclosed compounds to make them amenable to salt formation. For example, acidic functional groups can form stable salts with cations and basic functional groups can form stable salts with acids. Generally, there should be a difference of at least three units in the pKₐ (the logarithmic parameter of the dissociation constant Kₐ, which reflects the degree of ionization of a substance at a particular pH) of the parent drug and the counterion. For parent drug molecules that are very weakly basic, the choice of salt former is preferably a strong acid, such as hydrochloric (pKₐ=−6.1), sulfuric (pKₐ₁=−3.0, pKₐ₂=−1.96), or methanesulfonic (pKₐ=−1.2) to ensure protonation of the parent drug molecule. Parent drug molecules that are more highly basic can form salts with weaker acids, such as phosphoric (pKₐ₁=2.15, pKₐ₂=7.2, pKₐ₃=12.38), tartaric (pKₐ=2.93), acetic (pKₐ=4.76), and benzoic (pKₐ=4.2) acids. For very weakly acidic parent drug molecules, strongly basic cations, such as sodium (pKₐ=14.8), potassium (pKₐ=16.0), or calcium (pKₐ=12.9) are preferable to ensure deprotonation of the parent drug molecule. Parent drug molecules that are more acidic can form stable salts with weaker cations, such as zinc (pKₐ=8.96), choline (pKₐ=8.9), and dithanolamine (pKₐ=9.65). Representative functional groups suitable for stable salt formation, listed in view of relative acid/base strength from stronger acid to stronger base, include, but are not limited to, sulphonic acid (pKₐ₁=−1.2, pKₐ₂=−0.7), carboxylic acid (pKₐ₁=4.2, pKₐ₂=−4.7), imide (pKₐ=8.2), phenol, thiol (pKₐ=10), sulphonamide (pKₐ=10-11), amide (pKₐ=13-14), pyridine/pyridyl (pKₐ=5.2), imine (pKₐ=9.2), arylamine (pKₐ=9.3), alkylamine (pKₐ=9.8-11), amidine (pKₐ=12.4), guanidine (pKₐ=13.7), and quaternary ammonium. See Wermuth, C. G., The Practice of Medicinal Chemistry, 3rd ed., Elsevier, pp. 751-755 (2008).

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The compounds according to the disclosure are effective over a wide dosage range. For example, in treating adult humans, dosages from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the subject to be treated, the body weight of the subject to be treated, and the preference and experience of the attending physician. Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery. In a particular embodiment, the pharmaceutical composition is formulated for inhalation or oral administration.

For injection, the agents of the disclosure may be formulated and diluted in aqueous solutions, such as in physiologically compatible buffers, such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as, saline, preservatives, such as benzyl alcohol, absorption promoters, and fluorocarbons.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler, such as lactose, binders, such as starches, and/or lubricants, such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added. In some embodiments, the pharmaceutical composition is formulated for inhalation or oral administration.

C. Kits Comprising the Presently Disclosed Compounds

The presently disclosed subject matter also provides kits comprising at least one presently disclosed compound. In some embodiments, the presently disclosed subject matter provides a kit that can be used in combination with chemotherapeutic drugs and/or ionizing radiation, thereby increasing the efficacy of a chemotherapeutic drug(s) and/or ionizing radiation. In some embodiments, the kit can be used make cells less resistant to chemotherapeutic drugs and/or radiation therapy. In further embodiments, the kit comprises an effective amount of at least one of the presently disclosed Nrf2 inhibitors and written instructions for use of the kit. The kit may be comprised of at least one of the presently disclosed compounds and at least one chemotherapeutic drug or it may be comprised of at least one presently disclosed compounds and no chemotherapeutic drug.

In yet further embodiments, the presently disclosed subject matter provides a kit for treating cancer, the kit comprising a therapeutically effective amount of one of the presently disclosed Nrf2 inhibitors and written instructions for use of the kit.

II. Methods for Treating or Preventing a Disease, Disorder, or Condition Associated with an Nrf2-Regulated Pathway

In some embodiments, and as disclosed in more detail herein below, the presently disclosed subject matter provides a method for treating or preventing a disease, disorder or condition associated with an Nrf2-regulated pathway, the method comprising administering at least one presently disclosed Nrf2 inhibitor to the subject in an amount effective to decrease Nrf2 expression, thereby treating or preventing the disease, disorder, or condition.

In other embodiments, the presently disclosed methods comprise a method for treating or preventing a disease, disorder or condition associated with an Nrf2-regulated pathway, the method comprising administering at least one compound of formula (1), formula (2), or formula (3), as defined herein.

In some embodiments, the presently disclosed subject matter provides a combination therapy comprising a presently disclosed compound and a chemotherapeutic drug and/or a radiation therapy. In some embodiments, administration of a presently disclosed Nrf2 inhibitor occurs before administration of a chemotherapeutic drug and/or a radiation therapy. In other embodiments, administration of the Nrf2 inhibitor occurs at the same time as administration of a chemotherapeutic drug and/or a radiation therapy. In further embodiments, administration of the Nrf2 inhibitor occurs after administration of a chemotherapeutic drug and/or a radiation therapy. As such, the presently disclosed subject matter provides a method, wherein the compound is administered before, during, or after administration of a chemotherapeutic drug and/or a radiation therapy to the subject.

As provided herein, administration of a presently disclosed compound makes cancer cells less resistant to chemotherapy and/or radiation. As such, administration of a presently disclosed compound enhances the efficacy of a chemotherapeutic drug and/or a radiation therapy.

In some embodiments, the presently disclosed methods treat or prevent a disease, disorder, or condition associated with an Nrf2-regulated pathway, wherein the disease, disorder or condition is associated with a disregulated Nrf2 activity.

In other embodiments, the presently disclosed compounds can be administered in combination with another compound that affects an Nrf2-regulated gene to improve the efficacy of the other compound. The Nrf2-regulated gene may be a gene that encodes for an efflux transporter or a metabolic protein, for example. In still other embodiments, the combination of the compounds reduces the dosage required when compared to administering one compound by itself.

In some embodiments, the disease, disorder, or condition that is affected by a presently disclosed compound is cancer. In some embodiments, the chemotherapeutic drug is selected from the group consisting of a topoisomerase inhibitor, alkylating agent, antimetabolite, anthracycline, and plant alkoid. In other embodiments, the chemotherapeutic drug is selected from the group consisting of etoposide, cisplatin, paclitaxel, gemcitabine, and carboplatin. In still other embodiments, the compound is administered by inhalation or oral administration. In further embodiments, the methods suppress tumor growth. In still further embodiments, the method inhibits or prevents the metastasis of a tumor.

In some embodiments, a method of the presently disclosed subject matter treats or prevents a disease, disorder or condition associated with an Nrf2-regulated pathway by decreasing Nrf2 transcription, Nrf2 translation, and/or Nrf2 biological activity.

In other embodiments, a method of the presently disclosed subject matter provides a compound which decreases an Nrf2 biological activity selected from the group consisting of Nrf2 binding to an antioxidant-response element (ARE), nuclear accumulation of Nrf2, and the transcriptional induction of an Nrf2 target gene. By “Nrf2 expression or biological activity” is meant binding to an antioxidant-response element (ARE), nuclear accumulation of Nrf2, the transcriptional induction of Nrf2 target genes, binding of Nrf2 to a Keap1 polypeptide, and the like. In still other embodiments, the method treats or prevents a disease, disorder or condition associated with an Nrf2-regulated pathway, wherein the Nrf2 target gene is selected from the group consisting of MARCO, HO-1, NQO1, GCLm, GST α1, TrₓR₅ Pxr 1, GSR₅ G6PDH, GSS, GCLc, PGD, TKT, TALDO1, GST α3, GST p2, SOD2, SOD3, and GSR.

In some embodiments, the presently disclosed methods attenuate the expression of at least one cytoprotective gene. In other embodiments, the methods downregulate the expression of at least one chemoresistant or radioresistant gene. Nonlimiting examples of such genes include GCLm which encodes glutamate-cysteine ligase and NQO, a gene that encodes NAD(P)H dehydrogenase [quinone]1. In still other embodiments, the methods attenuate at least one drug efflux pathway, pathways comprised of pumps that extrude drugs and toxins out of a cell.

By expression of a gene, it is meant to refer to the mRNA levels, protein levels, and/or protein activity of a gene. In other words, it is meant to refer to the transcription, translation, and/or expression of a specific polypeptide or protein. By “attenuate”, “downregulate” or “decrease” the expression of a gene, it is meant that the mRNA levels, protein levels, and/or protein activity levels are less than when a presently disclosed compound is not administered.

As used herein, the term “disregulated Nrf2 expression” is meant to refer to a dysfunctional Nrf2 activity or level of activity.

“Cytoprotective” refers to providing protection to a cell against harmful agents. Therefore, a cytoprotective gene confers some protection to a cell against a harmful agent, such as a chemotherapeutic drug or radiation exposure. “Chemoresistance” refers to the resistance acquired by cells to the action of certain chemotherapeutic drugs. “Radioresistance” refers to the resistance acquired by cells to protect against ionizing radiation. In some embodiments, by decreasing the expression of at least one chemoresistant or radioresistant gene, the presently disclosed subject matter increases the efficacy of the chemotherapeutic drug and/or radiation treatment administered to a subject.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. It will be appreciated that, although not precluded, treating a disease, disorder or condition does not require that the disease, disorder, condition or symptoms associated therewith be completely eliminated.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

By “agent” is meant a presently disclosed compound or another agent, e.g., a peptide, nucleic acid molecule, or other small molecule compound administered in combination with a presently disclosed compound.

More particularly, the term “therapeutic agent” means a substance that has the potential of affecting the function of an organism. Such an agent may be, for example, a naturally occurring, semi-synthetic, or synthetic agent. For example, the therapeutic agent may be a drug that targets a specific function of an organism. A therapeutic agent also may be an antibiotic or a nutrient. A therapeutic agent may decrease, suppress, attenuate, diminish, arrest, or stabilize the development or progression of disease, disorder, or condition in a host organism.

The term “effective amount” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

An effective amount of a compound according to the presently disclosed methods can range from, e.g., about 0.001 mg/kg to about 1000 mg/kg, or in certain embodiments, about 0.01 mg/kg to about 100 mg/kg, or in certain embodiments, about 0.1 mg/kg to about 50 mg/kg. Effective doses also will vary, as recognized by those skilled in the art, depending on the disorder treated, route of administration, excipient usage, the age and sex of the subject, and the possibility of co-usage with other therapeutic treatments, such as use of other agents. It will be appreciated that an amount of a compound required for achieving the desired biological response may be different from the amount of compound effective for another purpose.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for treating an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Unless defined otherwise, all technical and scientific terms used herein have the meaning commonly understood by a person skilled in the art to which this invention belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Materials and Methods

### A549 NRF2-ARE-Fluc Stable Cell Line—

A549, the parental cell line, is a non-small-cell lung cancer (NSCLC) cell line with loss-of-function (LOF) Keap1 activity, thus NRF2 transcription factor constitutively translocates into the nucleus to activate the expression of downstream target genes of NRF2. A firefly luciferase reporter (Fluc) construct driven by a minimal promoter of NRF2-specific anti-oxidant responsive element (ARE) is stably expressed in the A549 cells. Compounds that reduce the translocation of NRF2 into the nucleus or prevent the interaction between NRF2 and ARE will lead to a decrease in luciferase activity.

### HEK293 CMV-Fluc Stable Cell Line—

This cell line has constitutively expressed Fluc under the control of the CMV promoter and was used in a counterscreen to remove general transcriptional modulators and general cytotoxic compounds.

### H838 NRF2-ARE-Fluc Stable Cell Line—

H838 is a NSCLC cell line with LOF Keap1 activity and constitutive translocation of NRF2 into the nucleus. A firefly luciferase reporter construct driven by a minimal promoter of NRF2-specific ARE is stably expressed in the H838 cells. This cell line was used as a confirmation assay to make sure hits identified from primary screening and that passed counterscreen assays worked in different cell types with a constitutively active NRF2 pathway (deficiency in Keap1).

### H1437 NRF2-ARE-Fluc Stable Cell Line—

H1437 is another NSCLC cell line with LOF Keap1 activity and constitutive translocation of NRF2 into the nucleus. A firefly luciferase reporter construct driven by a minimal promoter of NRF2-specific ARE is stably expressed in the H1437 cells. This cell line was used as a confirmation assay to make sure hits identified from primary screening and that passed counterscreen assays worked in different cell types with a constitutively active NRF2 pathway (deficiency in Keap1).

Primary Assay: Multiplexed NRF2 Reporter Gene and CellTiter-Fluor Cell Viability Assays in A549 Cells—

5 μL of A549 NRF2-ARE-Fluc cells at 4×10⁵ cell/mL in OPTI-MEM medium containing 5% FBS were dispensed into white solid 1536-well plates (Greiner Bio-One, Monroe, N. C.; Product #789173-F), and cultured at 37° C., 95% humidity, and 5% CO₂ for 2 hours. 23 nL of compounds dissolved in DMSO at different concentrations were transferred to the assay plates using a Kalypsys 1536-pin tool (Kalypsys, San Diego, Calif.). The final concentration of DMSO was maintained at 0.46%. Control compounds Budesonide and Staurosporine were both added at a concentration of 2 mM and Budesonide also was added as a 1:10 titration, starting at 200 μM, to achieve dose-response. After an 18-24 hour incubation at 37° C., 95% humidity, and 5% CO₂, 1 μL of 5× CellTiter-Fluor non-lytic cell viability assay reagent (Promega, Madison, Wis.) was added into the each well of the plates. The plates were then incubated for 30 minutes at room temperature before they were read on a ViewLux plate imager (Perkin Elmer, Waltham, Mass.) using an excitation wavelength of 405 nm and an emission wavelength of 525 nm. Finally, 2.5 L of luciferin-based detection reagent containing DTT, CoA, ATP (Sigma-Aldrich, St. Louis, Mo.; Product #D0632, C-3019, A-7699), and Luciferin (Biosynth AG, Itasca, Ill.; Product #L-8240) were added into each well, the plates were incubated for 15 minutes, and then were read on a ViewLux plate imager using the luminescent mode.

Counter Assay 1: Biochemical Firefly Luciferase Assay—

This assay was used to remove compounds that inhibit the luciferase reporter enzyme. 3 μL of substrate solution containing 50 mM Tris acetate, 13.3 mM Mg-acetate, 0.01 mM ATP, 0.01% Tween, 0.05% BSA and 0.01 mM D-Luciferin (Sigma-Aldrich, St. Louis, Mo.; Product #L9504) was dispensed into a 1536-well white solid bottom assay plate (Greiner Bio-One, Monroe, N. C.; Product #789173-F), followed by 23 nL of hit compounds dissolved in DMSO at different concentrations using a Kalypsys 1536-pin tool (Kalypsys, San Diego, Calif.). Then 1 μL of firefly luciferase reagent containing 50 mM Tris-acetate and 0.04 μM P. pyralis luciferase (Sigma-Aldrich, St. Louis, Mo.; Product #L9506) was added. The final DMSO concentration was maintained at 0.56%. After incubation at room temperature for 10 minutes, the plates were read by a Viewlux plate imager (PerkinElmer, Waltham, Mass.) using the luminescent mode.

Counter Assay 2: Multiplexed CMV Driven Luciferase Reporter Gene and CellTiter-Fluor Cell Viability Assays—

This assay was used to remove general transcriptional modulators and general cytotoxic compounds. The assay procedure was similar to the primary assay, except cell line and control compound were changed. 5 μL of HEK293-CMV-Fluc cells at 4×10⁵ cell/mL in OPTI-MEM medium containing 5% FBS were dispensed into white solid 1536-well plates (Greiner Bio-One, Monroe, N. C.; Product #789173-F), and cultured at 37° C., 95% humidity, and 5% CO₂ for 2 hours. 23 nL of hit compounds dissolved in DMSO at different concentrations were transferred to the assay plates using a Kalypsys 1536-pin tool (Kalypsys, San Diego, Calif.). The final concentration of DMSO was 0.46%. Control compound PTC124 was added to achieve a final concentration of 75 μM. After an 18-24 hour incubation at 37° C., 95% humidity, and 5% CO₂, 1 μL of 5× CellTiter-Fluor non-lytic cell viability assay reagent (Promega, Madison Wis.) was added into the each well of the plates. The plates were then incubated for 30 minutes at room temperature before they were read on a ViewLux plate imager (Perkin Elmer, Waltham, Mass.) using an excitation wavelength of 405 nm and an emission wavelength of 525 nm. Finally, 2.5 μL of luciferin-based detection reagent containing DTT, CoA, ATP (Sigma-Aldrich, St. Louis, Mo.; Product #D0632, C-3019, A-7699), and Luciferin (Biosynth AG, Itasca, Ill.; Product #L-8240) were added into each well, the plates were incubated for 15 minutes, and then were read on a ViewLux plate imager using the luminescent mode.

Confirmation Assay 1: Multiplexed NRF2 Reporter Gene and CellTiter-Fluor Cell Viability Assays in H838 Cells—

This assay was very similar to the primary assay except that the cell line was changed. 5 μL of H838 NRF2-ARE-Fluc cells at 4×10⁵ cell/mL in OPTI-MEM medium containing 5% FBS were dispensed into white solid 1536-well plates (Greiner Bio-One, Monroe, N. C.; Product #789173-F), and cultured at 37° C., 95% humidity, and 5% CO₂ for 2 hours. 23 nL of compounds dissolved in DMSO at different concentrations were transferred to the assay plates using a Kalypsys 1536-pin tool (Kalypsys, San Diego, Calif.). The final concentration of DMSO was maintained at 0.46%. Control compounds Budesonide and Staurosporine were both added at a concentration of 2 mM and Budesonide was also added as a 1:10 titration, starting at 200 μM, to achieve dose-response. After an 18-24 hour incubation at 37° C., 95% humidity, and 5% CO₂, 1 μL of 5× CellTiter-Fluor non-lytic cell viability assay reagent (Promega, Madison Wis.) was added into the each well of the plates. The plates were then incubated for 30 minutes at room temperature before they were read on a ViewLux plate imager (Perkin Elmer, Waltham Mass.) using an excitation wavelength of 405 nm and an emission wavelength of 525 nm. Finally, 2.5 L of luciferin-based detection reagent containing DTT, CoA, ATP (Sigma-Aldrich, St. Louis, Mo.; Product #D0632, C-3019, A-7699), and Luciferin (Biosynth AG, Itasca, Ill.; Product #L-8240) were added into each well, the plates were incubated for 15 minutes, and then were read on a ViewLux plate imager (Perkin Elmer, Waltham Mass.) using the luminescent mode.

Confirmation Assay 2: Multiplexed NRF2 Reporter Gene and CellTiter-Fluor Cell Viability Assays in H1437 Cells—

This assay was very similar to the primary assay except that the cell line was changed. 5 μL of H1437 NRF2-ARE-Fluc cells at 4×10⁵ cell/mL in OPTI-MEM medium containing 5% FBS were dispensed into white solid 1536-well plates (Greiner Bio-One, Monroe, N. C.; Product #789173-F), and cultured at 37° C., 95% humidity, and 5% CO₂ for 2 hours. 23 nL of compounds dissolved in DMSO at different concentrations were transferred to the assay plates using a Kalypsys 1536-pin tool (Kalypsys, San Diego, Calif.). The final concentration of DMSO was maintained at 0.46%. Control compounds Budesonide and Staurosporine were both added at a concentration of 2 mM and Budesonide was also added as a 1:10 titration, starting at 200 μM, to achieve dose-response. After an 18-24 hour incubation at 37° C., 95% humidity, and 5% CO₂, 1 μL of 5× CellTiter-Fluor non-lytic cell viability assay reagent (Promega, Madison Wis.) was added into the each well of the plates. The plates were then incubated for 30 minutes at room temperature before they were read on a ViewLux plate imager (Perkin Elmer, Waltham Mass.) using an excitation wavelength of 405 nm and an emission wavelength of 525 nm. Finally, 2.5 μL of luciferin-based detection reagent containing DTT, CoA, ATP (Sigma-Aldrich, St. Louis, Mo.; Product #D0632, C-3019, A-7699), and Luciferin (Biosynth AG, Itasca, Ill.; Product #L-8240) were added into each well, the plates were incubated for 15 minutes, then were read on a ViewLux plate imager using the luminescent mode.

### Example 2

### Screening Strategy to Identify Small Molecule Inhibitors of Nrf2

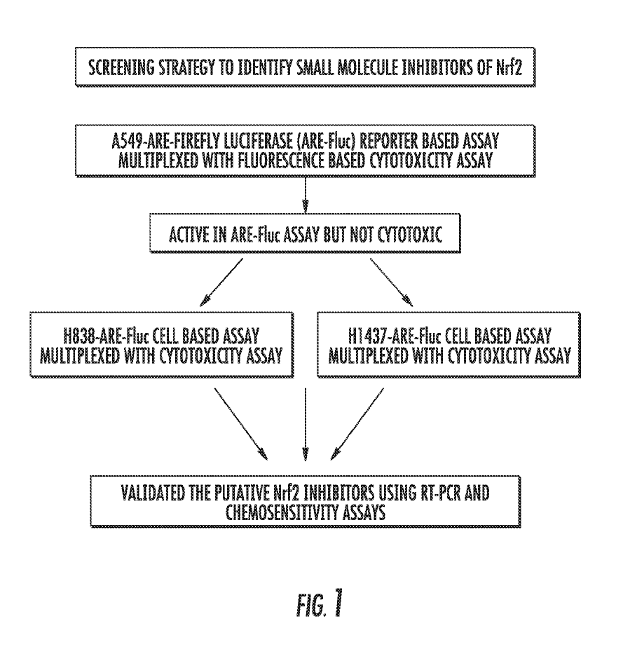

To identify potent and specific inhibitors of Nrf2, a library of approximately 400,000 small molecules maintained in the Molecular Probe Libraries Small Molecule Repository (MLPCN) program at National Institute of Health, USA was screened. A cell based reporter assay approach was used for the identification of agents that could inhibit Nrf2 mediated gene expression (FIG. 1). A549-ARE-luciferase cells express the luciferase gene driven by a minimal promoter and an enhancer element containing an NRF2 binding site (Antioxidant Response Element, ARE). Firefly luciferase reporter activity in A549-ARE-Luc cells was proportional to total NRF2 activity.

Briefly, lung adenocarcinoma cells (A549) that were stably transfected with the ARE-firefly luciferase (ARE-Fluc) reporter vector were plated in 1586-well plates. After overnight incubation, cells were pretreated for 16 h with different compounds. Budesonide, a steroid based inhibitor of Nrf2 was used as positive control.

Luciferase activity was measured after 16 h of drug treatment using the luciferase assay system from Promega (Madison, Wis.). Drug induced cytotoxicity was measured using a fluorescence-based cytotoxicity assay. The data were normalized for cell number and the decrease in luciferase activity, which reflects the degree of Nrf2 inhibition, was recorded. In this reporter assay based screening, the putative inhibitors were identified that suppressed NRF2 activity and resulted in reduced luminescent signal as compared to the vehicle treated cells.

Approximately 350,000 compounds were screened, with each compound tested at six different concentrations ranging from 38 μM to 2.4 nM using the NRF2 reporter cells. After the primary screen, 1452 putative NRF2 inhibitors were identified to be active in A549 cells. These 1452 drugs were rescreened in A549-ARE-luciferase cells using fresh aliquot of drug from the MLPCN library. Of these, 1312 drugs were confirmed as suppressors of NRF2 dependent luciferase activity in A549 cells.

To rule out non-specific inhibitors of luciferase or promoter activity scored as NRF2 inhibitors, 293T-CMV-luciferase cells, which express the luciferase gene driven by a constitutive promoter, were used. Additionally, these drugs were tested for luciferase inhibitory activity using an in vitro luciferase enzyme activity assay developed at NIH Chemical Genomics Center, NIH. Screening of these 1312 drugs in 293T-CMV-luciferase cells, as well as testing the drugs with the in vitro enzyme activity assay, filtered out 1072 compounds as non-specific inhibitors of transcription or luciferase activity.

The final 240 putative NRF2 inhibitors, which were not cytotoxic, were further screened for NRF2 inhibitory activity in two additional NSCLC cell lines harboring a Keap1 mutation (H838-ARE-luciferase and H1437-ARE-luciferase). Of these 240 drugs, 86 were found to be active only in A549 cells and 111 were active in at least two of the three cell lines. Compounds showing Nrf2 inhibitory activity in at least two cell lines were selected for detailed characterization.

For the real time reverse transcription polymerase chain reaction (RT-PCR) assay, cells were treated with Nrf2 inhibitors for 36-48 hrs, followed by total RNA isolation. The mRNA expression of Nrf2 and its downstream target genes, GCLm and NQO1 was measured by RT-PCR. (NC means no significant change in gene expression in comparison with vehicle group; M represents the concentration of drug used in the gene expression studies and it is twice the IC50 values generated from primary screening with A549-ARE reporter cell lines).

For the clonogenic assay, exponentially growing cells were counted, diluted, and seeded in triplicate at 1,000 cells/well in a 6-well plate. Cells were incubated for 24 h in a humidified CO₂ incubator at 37° C., and exposed to drugs or vehicle for 48 h. The chemotherapeutic drugs, etoposide, cisplatin, or carboplatin were added to some of the samples to see the effect of the drug in the presence and absence of the small inhibitors of Nrf2. After the treatment period, the drug containing media was replaced with complete growth media. To assess clonogenic survival following the drug treatment, A549 cells from a type of non-small cell lung cancer cell line were incubated in complete growth medium at 37° C. for 10-14 days and then stained with 50% methanol-crystal violet solution. Only colonies with more than 50 cells were counted (final concentration of DMSO in the growth media was 0.1%).

Studies suggest that Nrf2 forms heterodimers with small Maf proteins. To determine if the presently disclosed small molecule inhibitors were specifically interacting with Nrf2 or the Nrf2-MAF protein complex, a fluorescence polarization assay was developed. Ammonium aurintricarboxylate, a potent inhibitor of protein-nucleic acid interactions was used as positive control in the assay. The concentration of budesonide was 10 μM in the assay.

The cytotoxicity of the presently disclosed small compound inhibitors was analyzed by using a colorimetric methylthiazolydiphenyl-tetrazolium bromide (MTT) assay as described (Kumar et al., 2007; Singh et al., 2008). Briefly, the cells were treated with the small compound inhibitor or DMSO alone (0.1%, as vehicle) for 24 h. Four hours before the end of incubation, the medium was removed and 100 μL of MTT (5 mg/mL in serum free medium) was added to each well. The MTT was removed after 4 h, cells were washed with PBS, and 100 μL DMSO was added to each well to dissolve the water-insoluble MTT-formazan crystals. The absorbance was recorded at 570 nm in a plate reader (Molecular Devices, Sunnyvale, Calif.).

### Example 3

### Identification of Small Molecule Based Compounds as Inhibitors of Nrf2 Activity

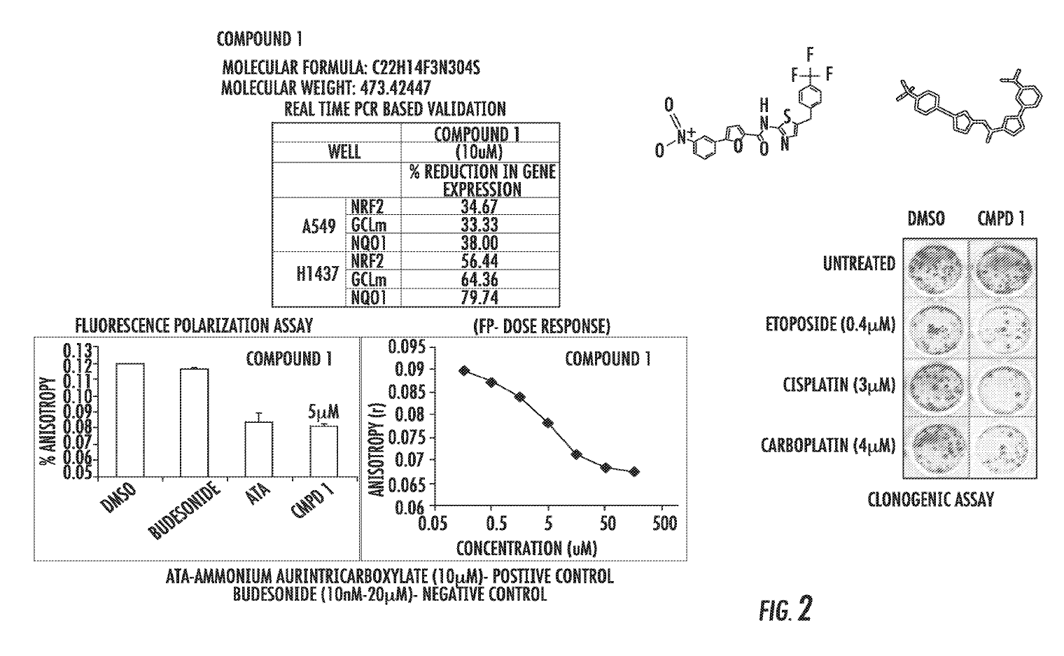

The presently disclosed subject matter provides small molecules that have been identified as inhibitors of Nrf2 activity. In general, these inhibitors showed a decrease in Nrf2 mRNA expression as well as a decrease in the expression of downstream target genes, GCLm and NQO1 as seen by the real time RT-PCR assays (see, e.g., FIGS. 2-4).

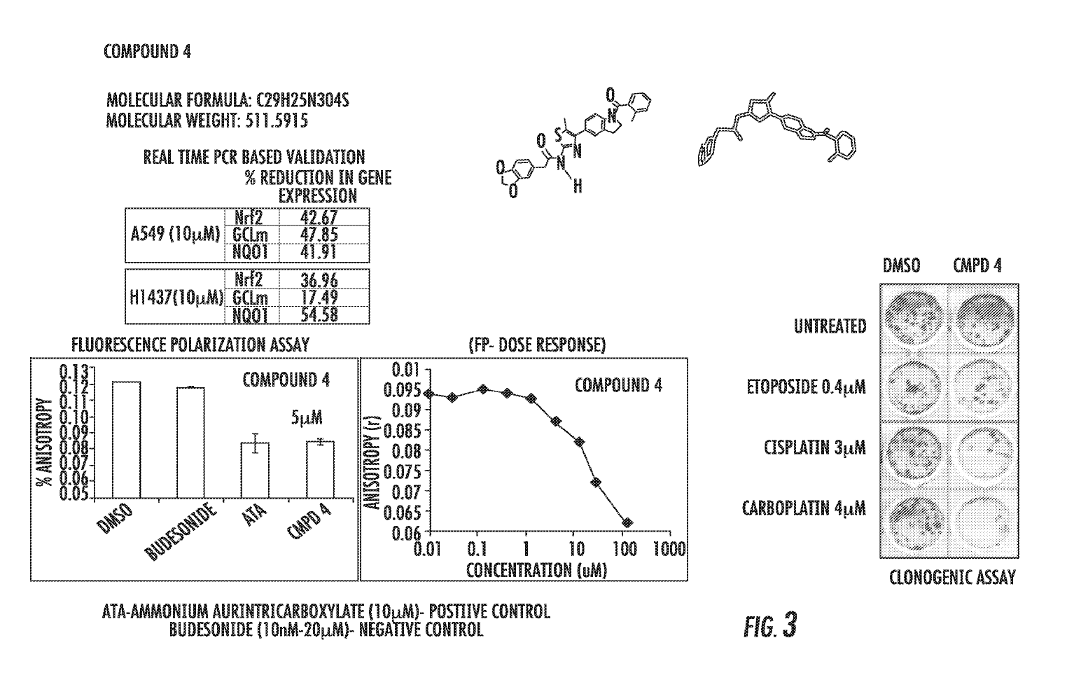

The clonogenic assays showed that these small molecule inhibitors of Nrf2 were more effective in combination with a chemotherapy drug in killing cancer cells compared to the chemotherapy drug alone (FIGS. 2 and 3). The inhibition of Nrf2 expression increased the sensitivity of cancer cells to chemotherapeutic drugs.

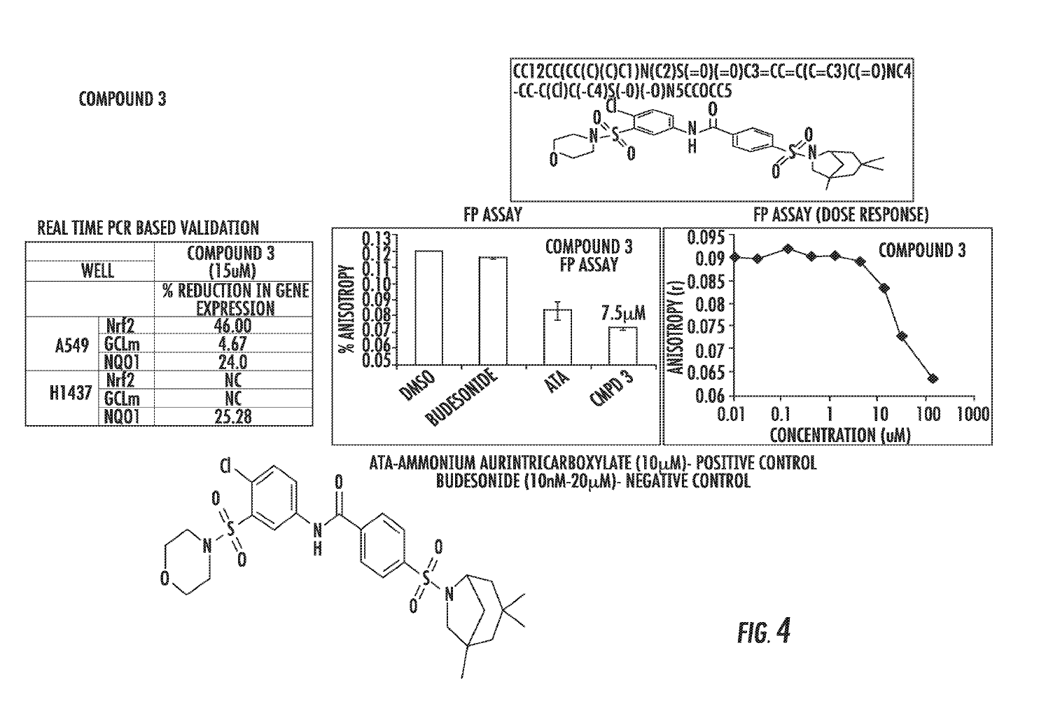

The Fluorescence Polarization (FP) assays showed the DNA binding activity of the Nrf2-MAF protein complex (FIGS. 2-4). FP assay data showing inhibition of binding of Nrf2-MAFG protein complex to fluorescein labeled ARE oligos in the presence of compound 1 (FIG. 2), compound 4 (FIG. 3), and compound 3 (FIG. 4) are shown. Ammonium aurintricarboxylate (ATA-10 μM), a well-known inhibitor of DNA binding activity, was included as positive control. Dose dependent reduction in binding of Nrf2-MAFG protein complex to fluorescein labeled ARE oligos in the presence of compound 1 (FIG. 2), compound 4 (FIG. 3), and compound 3 (FIG. 4) are also shown. X-axis represents increasing concentration of Nrf2 inhibitor.

The MTT assays indicated that cancer cells in the presence of a chemotherapeutic drug and a presently disclosed inhibitor of Nrf2 resulted in less viable cancer cells than cancer cells that were only contacted with the chemotherapeutic drug. In other words, the presently disclosed Nrf2 inhibitors increased the efficacy of the chemotherapeutic drugs in cancer cells.

It was discovered that tumor cells (lung, prostate, breast, skin, esophageal and gall bladder) manipulate the Nrf2 pathway for their survival against cytotoxic chemotherapeutic and radiotherapeutic agents and promote tumorigenesis. Gain of Nrf2 function in cancer cells has been identified herein as a novel and central determinant of outcome for patients with cancer treated with chemotherapy and/or radiation. Furthermore, it has been shown that abrogation of Nrf2 expression in cancer cells increases sensitivity to chemotherapeutic drug- and ionizing radiation induced cell death in vitro and in vivo. The presently disclosed subject matter provides small molecule-based potent and specific inhibitors of Nrf2 that are beneficial in treating aggressive, drug resistant tumors thereby improving the overall survival in patients with cancer.

Inhibition of Nrf2 expression by RNAi approach attenuated the expression of cytoprotective genes and drug efflux pathways involved in counteracting electrophiles, oxidative stress and detoxification of a broad spectrum of drugs and enhanced sensitivity to chemotherapeutic drugs and radiation-induced cell death in vitro and in vivo. In addition, knocking down Nrf2 expression greatly suppressed in vitro and in vivo tumor growth of prostate and lung cancer cells. In summary, it has been shown that the dysregulated Nrf2-Keap1 pathway is a novel determinant of chemoresistance/radioresistance and inhibition of Nrf2 signaling enhances the efficacy of chemotherapeutic and radiotherapy. These small molecule based Nrf2 inhibitors significantly enhanced the cytotoxicity and efficacy of standard chemotherapy drugs.

### Example 4

### Inhibition of Binding of the Nrf2-MafG Protein Complex to DNA Using Small Molecule Based Compounds

To determine the ability of the small molecule compounds to specifically inhibit the binding of an Nrf2-MafG protein complex to DNA, a fluorescence polarization (FP) assay was performed.

Briefly, fluorescein-labeled oligonucleotides corresponding to Anti-oxidant Response Element (ARE) were diluted to the appropriate concentration in PBS. Nrf2/MafG heterodimer was prepared by gel filtration with mixed samples of purified Nrf2 and MafG proteins. MafG/Nrf2 complex was then diluted with the buffer containing Nrf2 inhibitors or buffer only to the appropriate starting concentration and then serially diluted and incubated at 4° C. for 1 h. A mixture containing fluorescein-labeled ARE and purified protein sample was incubated at 4° C. for another 1 h. After pre-warming samples to 25° C. for 2 to 3 min, fluorescence anisotropy and total intensity was measured for each dilution using a FlexStation 3 (Molecular Devices, LLC) in Basic Binding Assay-FP mode. Ammonium aurintricarboxylate (ATA-10 μM), a well-known inhibitor of DNA binding activity, was included as positive control.

### Example 5

### Assays of Representative Compounds

Representative compounds were assayed to assess the potency of inhibition in an A549 NRF2-ARE-Fluc stable cell line (A549 Nrf2 assay), in an H838 NRF2-ARE-Fluc stable cell line (H838 Nrf2 assay), and in an H1437 NRF2-ARE-Fluc stable cell line (H1437 Nrf2 assay) (see Table 3). Legend for Table 3: A: ≦5 μM; B: 5-25 μM; and C: >25 μM.

TABLE 3
Potency Of Inhibition In An A549 NRF2-ARE-Fluc Stable Cell Line, In An H838 NRF2-ARE-Fluc
Stable Cell Line, And In An H1437 NRF2-ARE-Fluc Stable Cell Line

| Cmpd | Structure | A549 | H838 | H1437 |
|---|---|---|---|---|
| 1 | 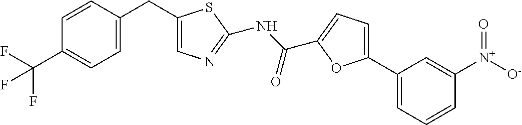 [SMILES: O=C(Nc1ncc(Cc2ccc(C(F)(F)F)cc2)s1)c1ccc(-c2cccc([N+](=O)[O-])c2)o1] | B | A | A |
| 2 | 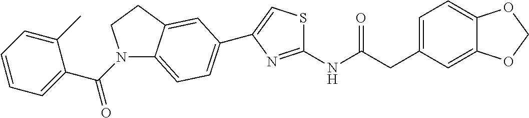 [SMILES: Cc1ccccc1C(=O)N1CCc2cc(-c3csc(NC(=O)Cc4ccc5c(c4)OCO5)n3)ccc21] | B | N/A | B |
| 3 | 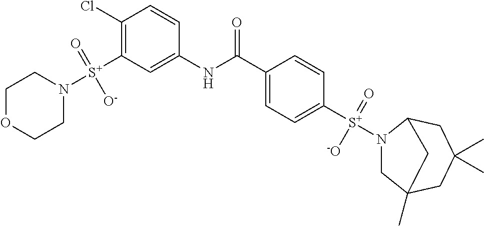 | A | A | A |
| 4 | 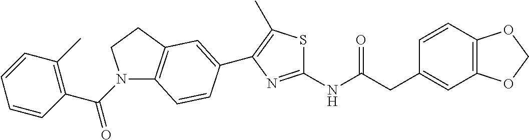 [SMILES: Cc1ccccc1C(=O)N1CCc2cc(-c3nc(NC(=O)Cc4ccc5c(c4)OCO5)sc3C)ccc21] | A | B | A |
| 5 | 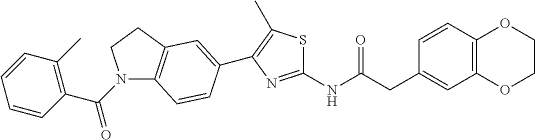 [SMILES: Cc1ccccc1C(=O)N1CCc2cc(-c3nc(NC(=O)Cc4ccc5c(c4)OCCO5)sc3C)ccc21] | B | N/A | A |
| 6 | 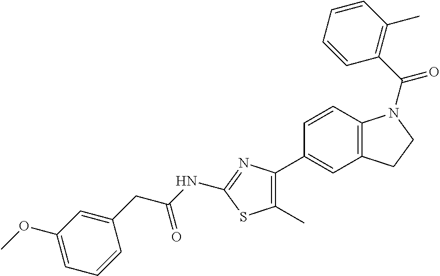 [SMILES: COc1cccc(CC(=O)Nc2nc(-c3ccc4c(c3)CCN4C(=O)c3ccccc3C)c(C)s2)c1] | A | N/A | A |
| 7 | 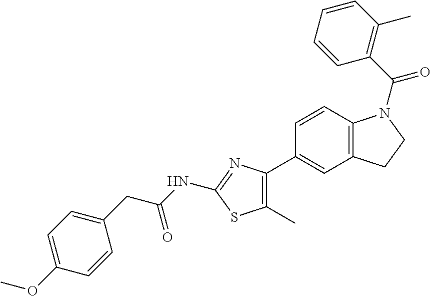 [SMILES: COc1ccc(CC(=O)Nc2nc(-c3ccc4c(c3)CCN4C(=O)c3ccccc3C)c(C)s2)cc1] | A | N/A | A |
| 8 | 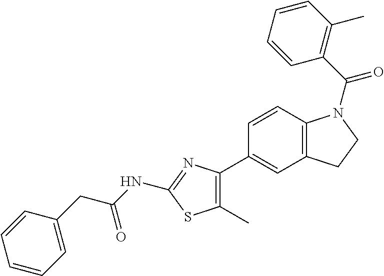 [SMILES: Cc1ccccc1C(=O)N1CCc2cc(-c3nc(NC(=O)Cc4ccccc4)sc3C)ccc21] | A | N/A | A |
| 9 | 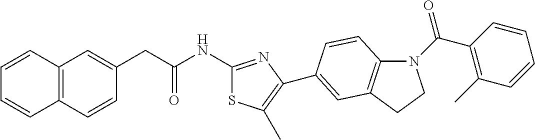 [SMILES: Cc1ccccc1C(=O)N1CCc2cc(-c3nc(NC(=O)Cc4ccc5ccccc5c4)sc3C)ccc21] | A | N/A | B |
| 10 | 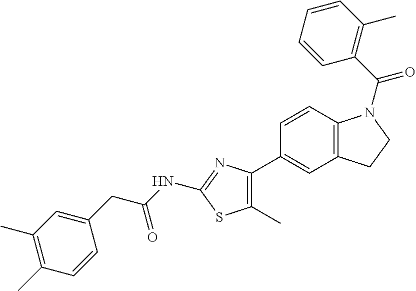 [SMILES: Cc1ccc(CC(=O)Nc2nc(-c3ccc4c(c3)CCN4C(=O)c3ccccc3C)c(C)s2)cc1C] | B | N/A | C |
| 11 | 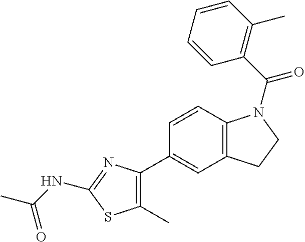 [SMILES: CC(=O)Nc1nc(-c2ccc3c(c2)CCN3C(=O)c2ccccc2C)c(C)s1] | B | N/A | A |
| 12 | 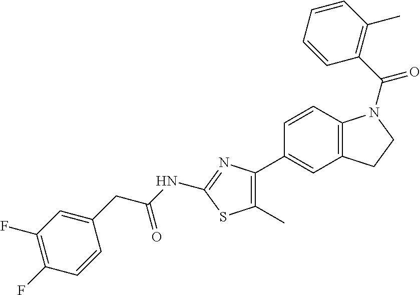 [SMILES: Cc1ccccc1C(=O)N1CCc2cc(-c3nc(NC(=O)Cc4ccc(F)c(F)c4)sc3C)ccc21] | A | N/A | A |
| 13 | 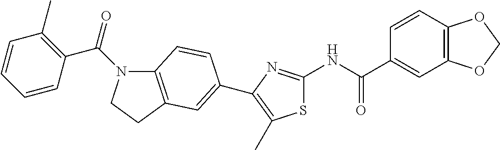 [SMILES: Cc1ccccc1C(=O)N1CCc2cc(-c3nc(NC(=O)c4ccc5c(c4)OCO5)sc3C)ccc21] | A | N/A | B |
| 14 | 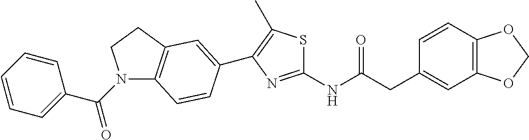 [SMILES: Cc1sc(NC(=O)Cc2ccc3c(c2)OCO3)nc1-c1ccc2c(c1)CCN2C(=O)c1ccccc1] | A | N/A | B |
| 15 | 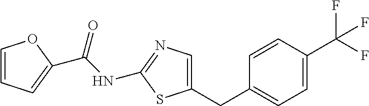 [SMILES: O=C(Nc1ncc(Cc2ccc(C(F)(F)F)cc2)s1)c1ccco1] | C | N/A | C |
| 16 | 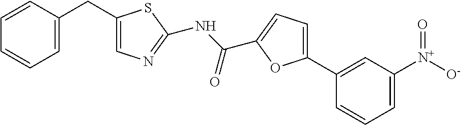 [SMILES: O=C(Nc1ncc(Cc2ccccc2)s1)c1ccc(-c2cccc([N+](=O)[O-])c2)o1] | B | N/A | B |
| 17 | 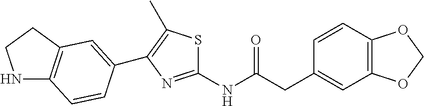 [SMILES: Cc1sc(NC(=O)Cc2ccc3c(c2)OCO3)nc1-c1ccc2c(c1)CCN2] | B | N/A | C |
| 18 | 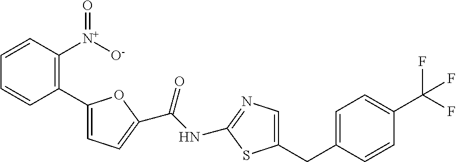 [SMILES: O=C(Nc1ncc(Cc2ccc(C(F)(F)F)cc2)s1)c1ccc(-c2ccccc2[N+](=O)[O-])o1] | B | N/A | A |
| 19 | 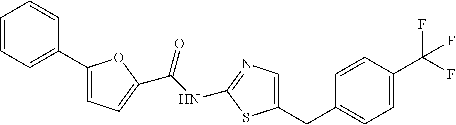 [SMILES: O=C(Nc1ncc(Cc2ccc(C(F)(F)F)cc2)s1)c1ccc(-c2ccccc2)o1] | B | N/A | C |
| 20 | 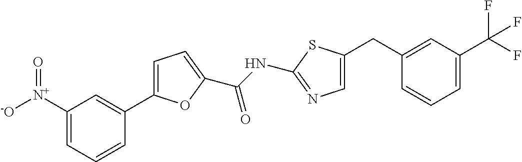 | B | N/A | A |
| 21 | 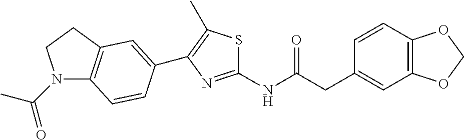 [SMILES: CC(=O)N1CCc2cc(-c3nc(NC(=O)Cc4ccc5c(c4)OCO5)sc3C)ccc21] | A | N/A | B |
| 22 | 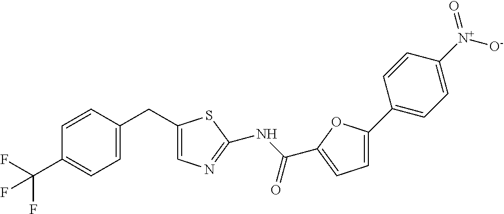 [SMILES: O=C(Nc1ncc(Cc2ccc(C(F)(F)F)cc2)s1)c1ccc(-c2ccc([N+](=O)[O-])cc2)o1] | B | N/A | B |
| 23 | 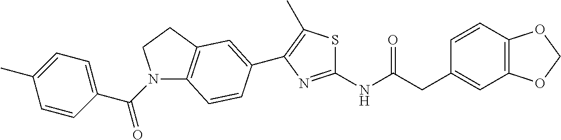 [SMILES: Cc1ccc(C(=O)N2CCc3cc(-c4nc(NC(=O)Cc5ccc6c(c5)OCO6)sc4C)ccc32)cc1] | B | N/A | C |
| 24 | 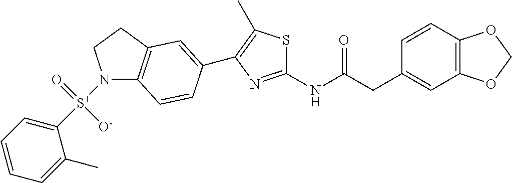 [SMILES: Cc1ccccc1[S+](=O)([O-])N1CCc2cc(-c3nc(NC(=O)Cc4ccc5c(c4)OCO5)sc3C)ccc21] | A | N/A | B |
| 25 | 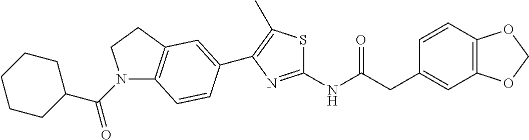 [SMILES: Cc1sc(NC(=O)Cc2ccc3c(c2)OCO3)nc1-c1ccc2c(c1)CCN2C(=O)C1CCCCC1] | B | N/A | A |
| 26 | 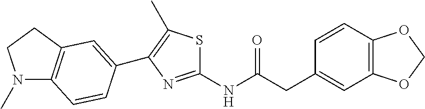 [SMILES: Cc1sc(NC(=O)Cc2ccc3c(c2)OCO3)nc1-c1ccc2c(c1)CCN2C] | B | N/A | C |
| 27 | 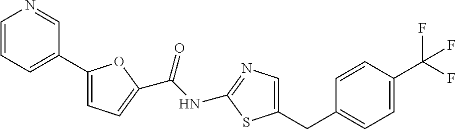 [SMILES: O=C(Nc1ncc(Cc2ccc(C(F)(F)F)cc2)s1)c1ccc(-c2cccnc2)o1] | B | N/A | A |
| 28 | 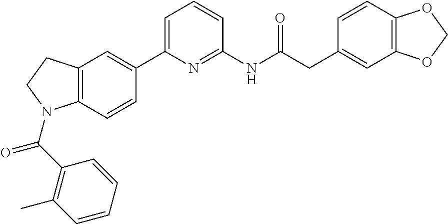 [SMILES: Cc1ccccc1C(=O)N1CCc2cc(-c3cccc(NC(=O)Cc4ccc5c(c4)OCO5)n3)ccc21] | B | N/A | C |
| 29 | 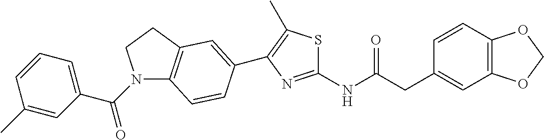 [SMILES: Cc1cccc(C(=O)N2CCc3cc(-c4nc(NC(=O)Cc5ccc6c(c5)OCO6)sc4C)ccc32)c1] | C | N/A | B |
| 30 | 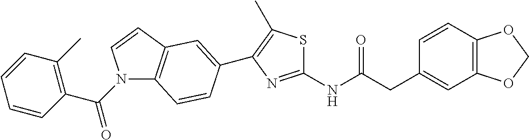 [SMILES: Cc1ccccc1C(=O)n1ccc2cc(-c3nc(NC(=O)Cc4ccc5c(c4)OCO5)sc3C)ccc21] | B | N/A | B |
| 31 | 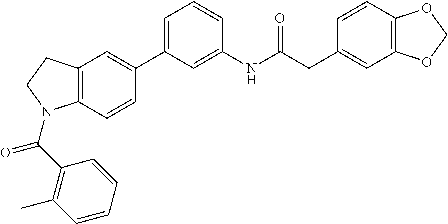 [SMILES: Cc1ccccc1C(=O)N1CCc2cc(-c3cccc(NC(=O)Cc4ccc5c(c4)OCO5)c3)ccc21] | B | N/A | C |
| 32 | 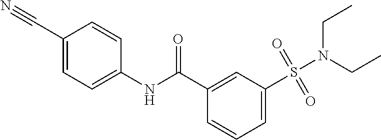 [SMILES: CCN(CC)S(=O)(=O)c1cccc(C(=O)Nc2ccc(C#N)cc2)c1] | A | N/A | B |
| 33 | 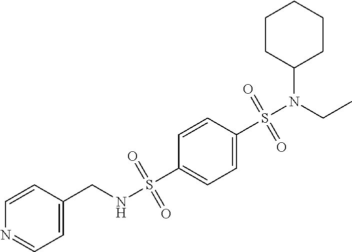 [SMILES: CCN(C1CCCCC1)S(=O)(=O)c1ccc(S(=O)(=O)NCc2ccncc2)cc1] | B | N/A | A |
| 34 | 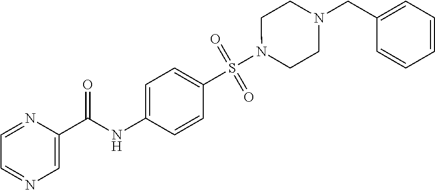 [SMILES: O=C(Nc1ccc(S(=O)(=O)N2CCN(Cc3ccccc3)CC2)cc1)c1cnccn1] | A | N/A | B |
| 35 | 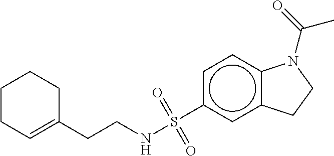 | A | N/A | B |
| 36 | 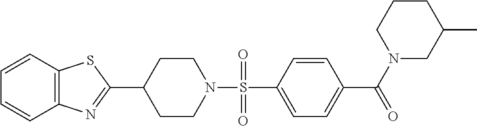 [SMILES: CC1CCCN(C(=O)c2ccc(S(=O)(=O)N3CCC(c4nc5ccccc5s4)CC3)cc2)C1] | A | N/A | A |
| 37 | 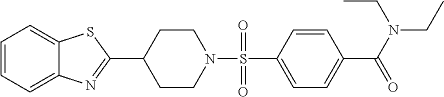 [SMILES: CCN(CC)C(=O)c1ccc(S(=O)(=O)N2CCC(c3nc4ccccc4s3)CC2)cc1] | A | N/A | A |
| 38 | 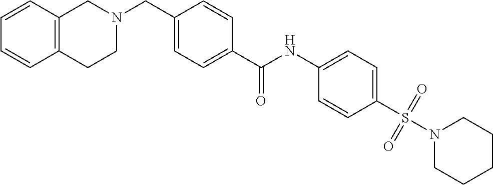 [SMILES: O=C(Nc1ccc(S(=O)(=O)N2CCCCC2)cc1)c1ccc(CN2CCc3ccccc3C2)cc1] | A | N/A | A |
| 39 | 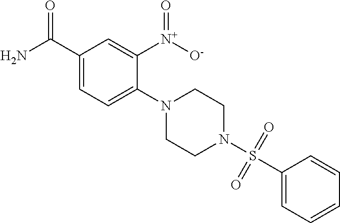 [SMILES: NC(=O)c1ccc(N2CCN(S(=O)(=O)c3ccccc3)CC2)c([N+](=O)[O-])c1] | A | N/A | B |
| 40 | 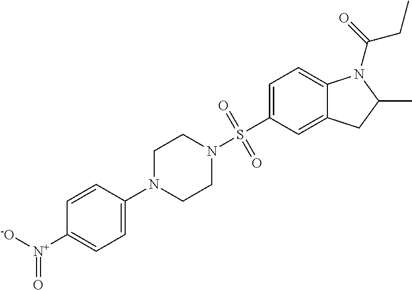 | A | N/A | A |
| 41 | 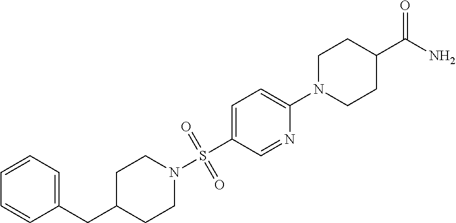 [SMILES: NC(=O)C1CCN(c2ccc(S(=O)(=O)N3CCC(Cc4ccccc4)CC3)cn2)CC1] | A | N/A | B |
| 42 | 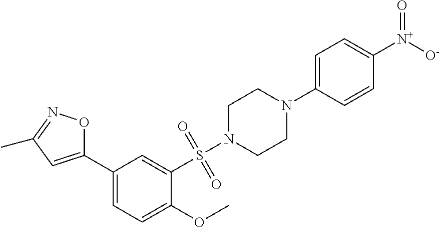 [SMILES: COc1ccc(-c2cc(C)no2)cc1S(=O)(=O)N1CCN(c2ccc([N+](=O)[O-])cc2)CC1] | A | N/A | A |
| 43 | 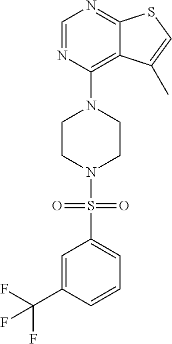 [SMILES: Cc1csc2ncnc(N3CCN(S(=O)(=O)c4cccc(C(F)(F)F)c4)CC3)c12] | B | N/A | A |
| 44 | 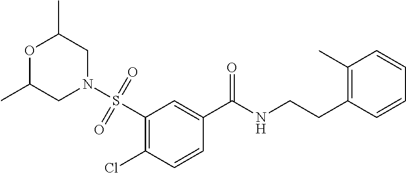 [SMILES: Cc1ccccc1CCNC(=O)c1ccc(Cl)c(S(=O)(=O)N2CC(C)OC(C)C2)c1] | B | N/A | B |
| 45 | 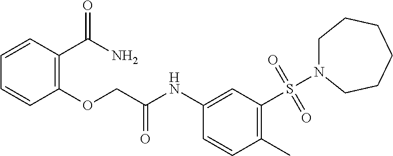 [SMILES: Cc1ccc(NC(=O)COc2ccccc2C(N)=O)cc1S(=O)(=O)N1CCCCCC1] | A | N/A | A |
| 46 | 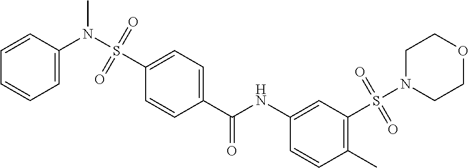 [SMILES: Cc1ccc(NC(=O)c2ccc(S(=O)(=O)N(C)c3ccccc3)cc2)cc1S(=O)(=O)N1CCOCC1] | A | N/A | B |
| 47 | 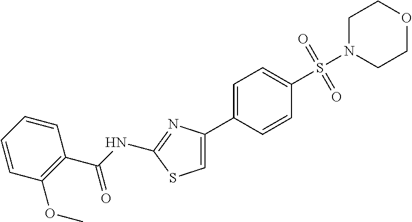 [SMILES: COc1ccccc1C(=O)Nc1nc(-c2ccc(S(=O)(=O)N3CCOCC3)cc2)cs1] | A | A | A |
| 48 | 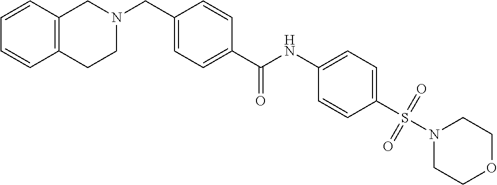 [SMILES: O=C(Nc1ccc(S(=O)(=O)N2CCOCC2)cc1)c1ccc(CN2CCc3ccccc3C2)cc1] | A | C | B |
| 49 | 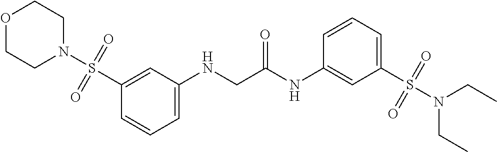 [SMILES: CCN(CC)S(=O)(=O)c1cccc(NC(=O)CNc2cccc(S(=O)(=O)N3CCOCC3)c2)c1] | A | C | A |
| 50 | 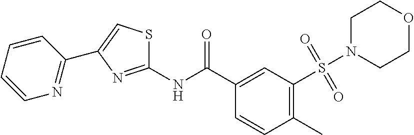 [SMILES: Cc1ccc(C(=O)Nc2nc(-c3ccccn3)cs2)cc1S(=O)(=O)N1CCOCC1] | A | C | C |
| 51 | 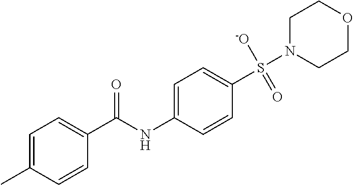 | B | N/A | C |
| 52 | 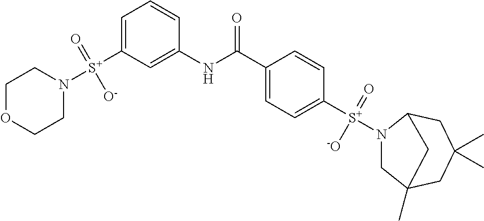 | A | N/A | A |
| 53 | 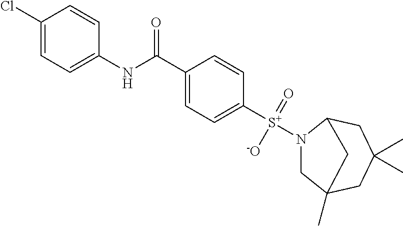 | A | N/A | B |
| 54 | 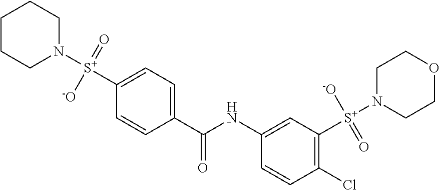 | A | N/A | A |
| 55 | 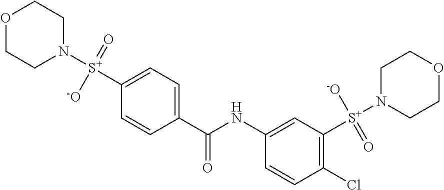 | A | N/A | A |
| 56 | 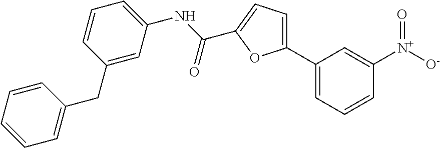 [SMILES: O=C(Nc1cccc(Cc2ccccc2)c1)c1ccc(-c2cccc([N+](=O)[O-])c2)o1] | B | N/A | B |
| 57 | 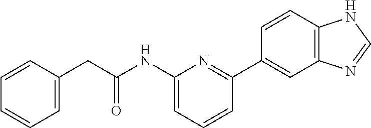 [SMILES: O=C(Cc1ccccc1)Nc1cccc(-c2ccc3[nH]cnc3c2)n1] | C | N/A | C |
| 58 | 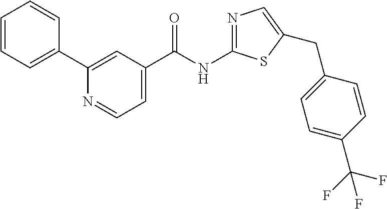 [SMILES: O=C(Nc1ncc(Cc2ccc(C(F)(F)F)cc2)s1)c1ccnc(-c2ccccc2)c1] | C | N/A | B |
| 59 | 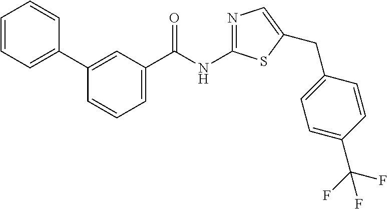 [SMILES: O=C(Nc1ncc(Cc2ccc(C(F)(F)F)cc2)s1)c1cccc(-c2ccccc2)c1] | B | N/A | C |
| 60 | 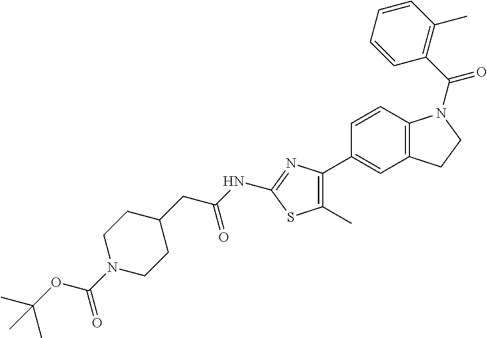 [SMILES: Cc1ccccc1C(=O)N1CCc2cc(-c3nc(NC(=O)CC4CCN(C(=O)OC(C)(C)C)CC4)sc3C)ccc21] | A | N/A | A |
| 61 | 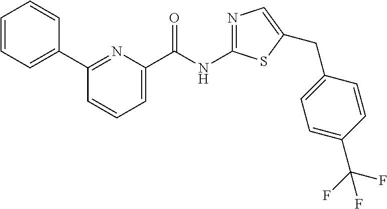 [SMILES: O=C(Nc1ncc(Cc2ccc(C(F)(F)F)cc2)s1)c1cccc(-c2ccccc2)n1] | A | N/A | C |
| 62 | 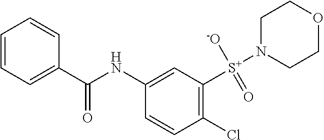 | B | N/A | B |
| 63 | 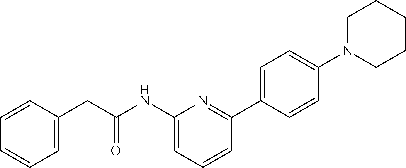 [SMILES: O=C(Cc1ccccc1)Nc1cccc(-c2ccc(N3CCCCC3)cc2)n1] | C | N/A | C |
| 64 | 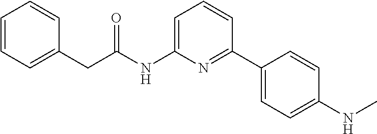 [SMILES: CNc1ccc(-c2cccc(NC(=O)Cc3ccccc3)n2)cc1] | B | N/A | C |
| 65 | 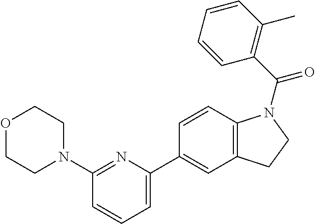 [SMILES: Cc1ccccc1C(=O)N1CCc2cc(-c3cccc(N4CCOCC4)n3)ccc21] | B | N/A | B |
| 66 | 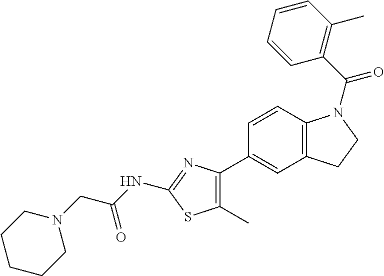 [SMILES: Cc1ccccc1C(=O)N1CCc2cc(-c3nc(NC(=O)CN4CCCCC4)sc3C)ccc21] | B | N/A | B |
| 67 | 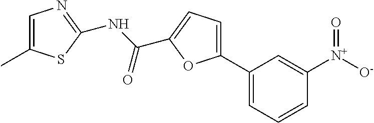 [SMILES: Cc1cnc(NC(=O)c2ccc(-c3cccc([N+](=O)[O-])c3)o2)s1] | B | N/A | C |
| 68 | 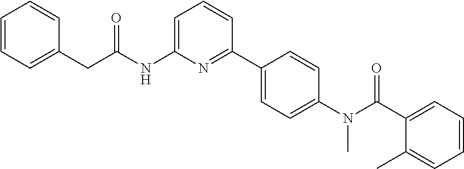 [SMILES: Cc1ccccc1C(=O)N(C)c1ccc(-c2cccc(NC(=O)Cc3ccccc3)n2)cc1] | B | N/A | B |
| 69 | 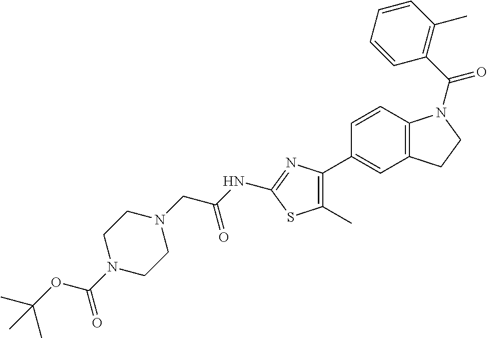 [SMILES: Cc1ccccc1C(=O)N1CCc2cc(-c3nc(NC(=O)CN4CCN(C(=O)OC(C)(C)C)CC4)sc3C)ccc21] | A | N/A | A |
| 70 | 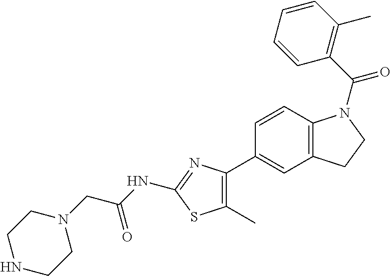 [SMILES: Cc1ccccc1C(=O)N1CCc2cc(-c3nc(NC(=O)CN4CCNCC4)sc3C)ccc21] | C | N/A | C |
| 71 | 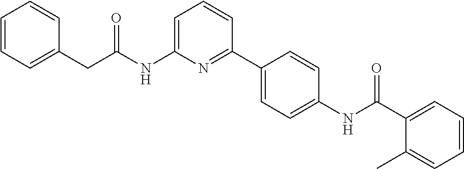 [SMILES: Cc1ccccc1C(=O)Nc1ccc(-c2cccc(NC(=O)Cc3ccccc3)n2)cc1] | B | N/A | C |
| 72 | 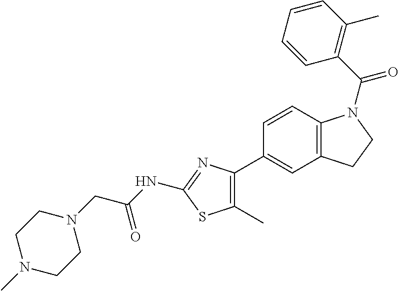 [SMILES: Cc1ccccc1C(=O)N1CCc2cc(-c3nc(NC(=O)CN4CCN(C)CC4)sc3C)ccc21] | C | N/A | B |
| 73 | 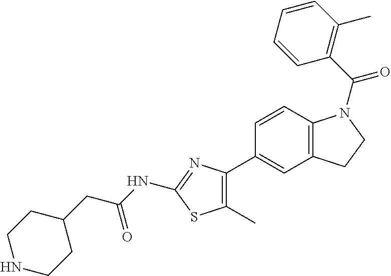 [SMILES: Cc1ccccc1C(=O)N1CCc2cc(-c3nc(NC(=O)CC4CCNCC4)sc3C)ccc21] | C | N/A | B |
| 74 | 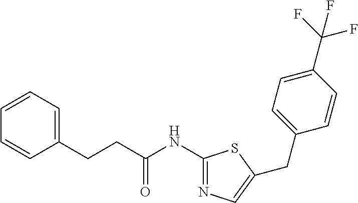 [SMILES: O=C(CCc1ccccc1)Nc1ncc(Cc2ccc(C(F)(F)F)cc2)s1] | C | N/A | C |
| 75 | 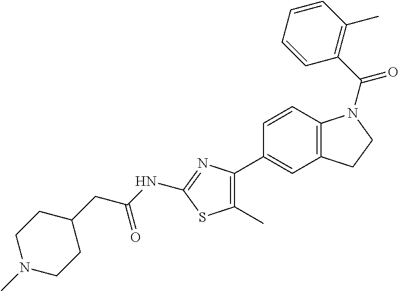 [SMILES: Cc1ccccc1C(=O)N1CCc2cc(-c3nc(NC(=O)CC4CCN(C)CC4)sc3C)ccc21] | B | N/A | B |
| 76 | 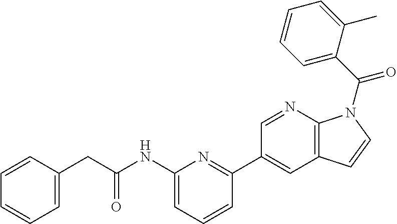 [SMILES: Cc1ccccc1C(=O)n1ccc2cc(-c3cccc(NC(=O)Cc4ccccc4)n3)cnc21] | B | N/A | C |
| 77 | 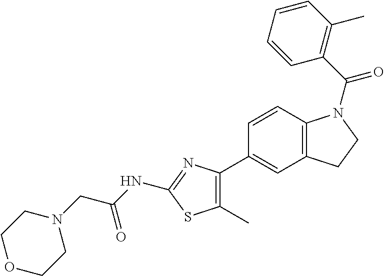 [SMILES: Cc1ccccc1C(=O)N1CCc2cc(-c3nc(NC(=O)CN4CCOCC4)sc3C)ccc21] | B | N/A | B |
| 78 | 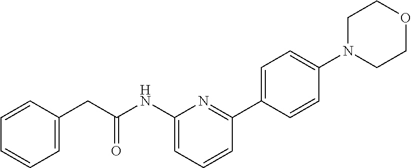 [SMILES: O=C(Cc1ccccc1)Nc1cccc(-c2ccc(N3CCOCC3)cc2)n1] | C | N/A | C |
| 79 | 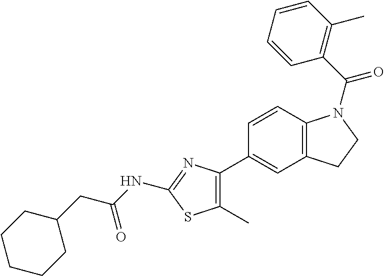 [SMILES: Cc1ccccc1C(=O)N1CCc2cc(-c3nc(NC(=O)CC4CCCCC4)sc3C)ccc21] | A | N/A | B |
| 80 | 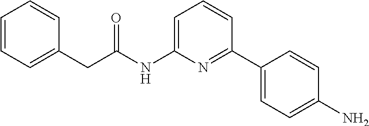 [SMILES: Nc1ccc(-c2cccc(NC(=O)Cc3ccccc3)n2)cc1] | C | N/A | C |
| 81 | 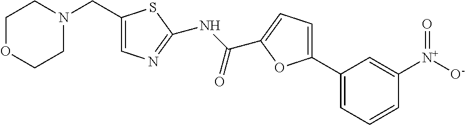 [SMILES: O=C(Nc1ncc(CN2CCOCC2)s1)c1ccc(-c2cccc([N+](=O)[O-])c2)o1] | B | N/A | B |
| 82 | 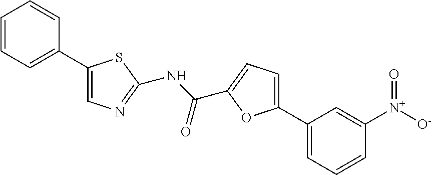 [SMILES: O=C(Nc1ncc(-c2ccccc2)s1)c1ccc(-c2cccc([N+](=O)[O-])c2)o1] | B | N/A | C |
| 83 | 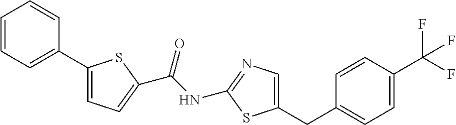 [SMILES: O=C(Nc1ncc(Cc2ccc(C(F)(F)F)cc2)s1)c1ccc(-c2ccccc2)s1] | C | N/A | C |
| 84 | 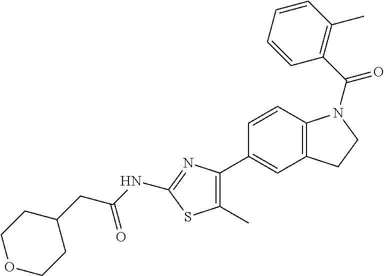 [SMILES: Cc1ccccc1C(=O)N1CCc2cc(-c3nc(NC(=O)CC4CCOCC4)sc3C)ccc21] | B | N/A | B |
| 85 | 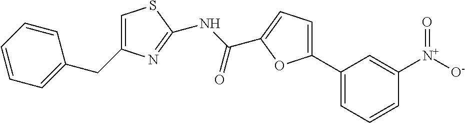 [SMILES: O=C(Nc1nc(Cc2ccccc2)cs1)c1ccc(-c2cccc([N+](=O)[O-])c2)o1] | B | N/A | C |
| 86 | 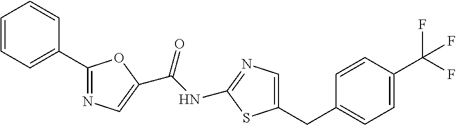 [SMILES: O=C(Nc1ncc(Cc2ccc(C(F)(F)F)cc2)s1)c1cnc(-c2ccccc2)o1] | B | N/A | B |
| 87 | 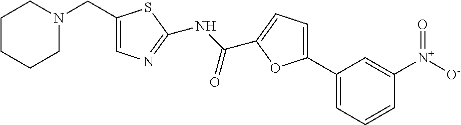 [SMILES: O=C(Nc1ncc(CN2CCCCC2)s1)c1ccc(-c2cccc([N+](=O)[O-])c2)o1] | C | N/A | C |
| 88 | 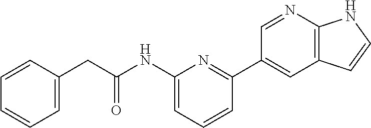 [SMILES: O=C(Cc1ccccc1)Nc1cccc(-c2cnc3[nH]ccc3c2)n1] | B | N/A | C |
| 89 | 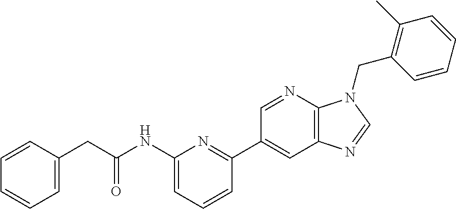 [SMILES: Cc1ccccc1Cn1cnc2cc(-c3cccc(NC(=O)Cc4ccccc4)n3)cnc21] | A | N/A | B |
| | 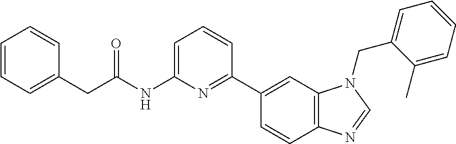 [SMILES: Cc1ccccc1Cn1cnc2ccc(-c3cccc(NC(=O)Cc4ccccc4)n3)cc21] | | | |
| 90 | 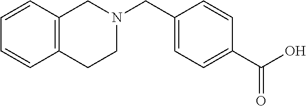 [SMILES: O=C(O)c1ccc(CN2CCc3ccccc3C2)cc1] | C | N/A | C |
| 91 | 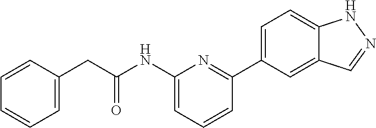 [SMILES: O=C(Cc1ccccc1)Nc1cccc(-c2ccc3[nH]ncc3c2)n1] | B | N/A | C |
| 92 | 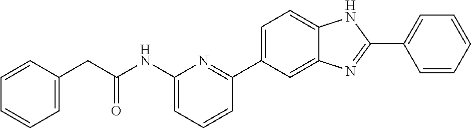 [SMILES: O=C(Cc1ccccc1)Nc1cccc(-c2ccc3[nH]c(-c4ccccc4)nc3c2)n1] | B | N/A | C |
| 93 | 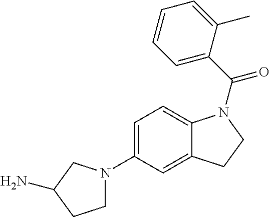 [SMILES: Cc1ccccc1C(=O)N1CCc2cc(N3CCC(N)C3)ccc21] | B | N/A | C |
| 94 | 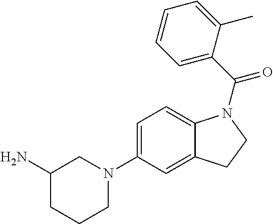 [SMILES: Cc1ccccc1C(=O)N1CCc2cc(N3CCCC(N)C3)ccc21] | C | N/A | C |
| 95 | 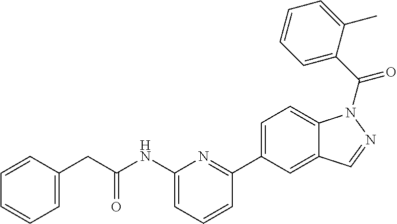 [SMILES: Cc1ccccc1C(=O)n1ncc2cc(-c3cccc(NC(=O)Cc4ccccc4)n3)ccc21] | A | N/A | B |
| 96 | 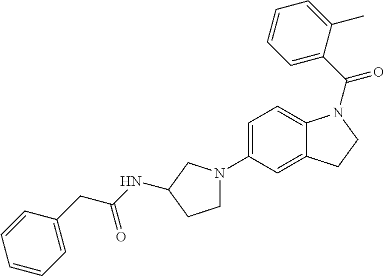 [SMILES: Cc1ccccc1C(=O)N1CCc2cc(N3CCC(NC(=O)Cc4ccccc4)C3)ccc21] | B | N/A | B |
| 97 | 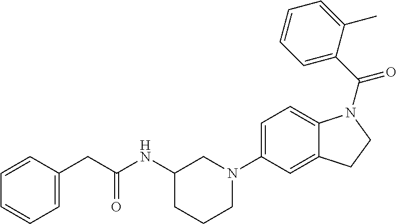 [SMILES: Cc1ccccc1C(=O)N1CCc2cc(N3CCCC(NC(=O)Cc4ccccc4)C3)ccc21] | B | N/A | B |
| 98 | 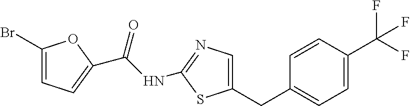 [SMILES: O=C(Nc1ncc(Cc2ccc(C(F)(F)F)cc2)s1)c1ccc(Br)o1] | B | N/A | B |
| 99 | 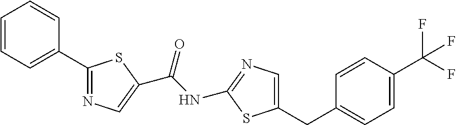 [SMILES: O=C(Nc1ncc(Cc2ccc(C(F)(F)F)cc2)s1)c1cnc(-c2ccccc2)s1] | B | N/A | C |
| 100 | 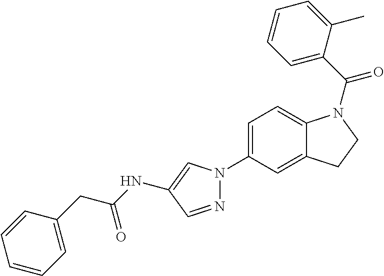 [SMILES: Cc1ccccc1C(=O)N1CCc2cc(-n3cc(NC(=O)Cc4ccccc4)cn3)ccc21] | B | N/A | B |
| 101 | 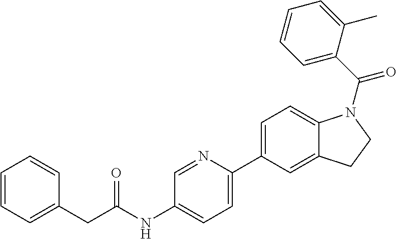 [SMILES: Cc1ccccc1C(=O)N1CCc2cc(-c3ccc(NC(=O)Cc4ccccc4)cn3)ccc21] | B | N/A | B |
| 102 | 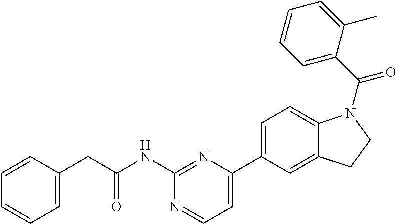 [SMILES: Cc1ccccc1C(=O)N1CCc2cc(-c3ccnc(NC(=O)Cc4ccccc4)n3)ccc21] | B | N/A | C |
| 103 | 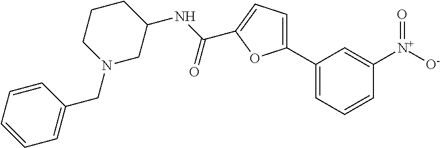 [SMILES: O=C(NC1CCCN(Cc2ccccc2)C1)c1ccc(-c2cccc([N+](=O)[O-])c2)o1] | C | N/A | C |
| 104 | 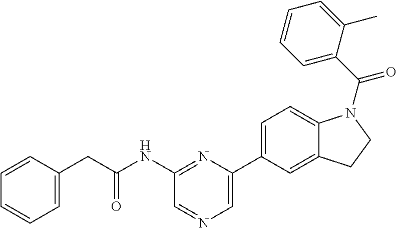 [SMILES: Cc1ccccc1C(=O)N1CCc2cc(-c3cncc(NC(=O)Cc4ccccc4)n3)ccc21] | A | N/A | B |
| 105 | 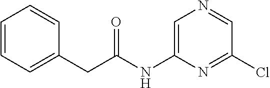 [SMILES: O=C(Cc1ccccc1)Nc1cncc(Cl)n1] | C | N/A | C |
| 106 | 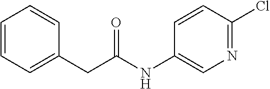 [SMILES: O=C(Cc1ccccc1)Nc1ccc(Cl)nc1] | C | N/A | C |
| 107 | 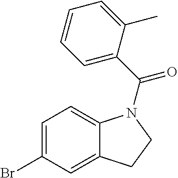 [SMILES: Cc1ccccc1C(=O)N1CCc2cc(Br)ccc21] | B | N/A | C |
| 108 | 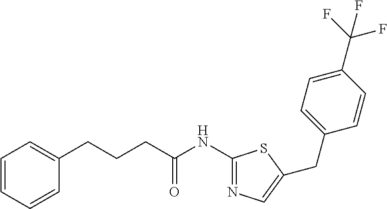 [SMILES: O=C(CCCc1ccccc1)Nc1ncc(Cc2ccc(C(F)(F)F)cc2)s1] | C | N/A | C |
| 109 | 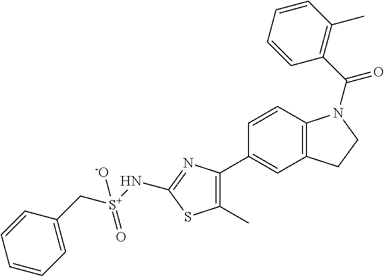 | B | N/A | B |
| 110 | 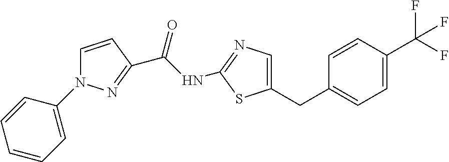 [SMILES: O=C(Nc1ncc(Cc2ccc(C(F)(F)F)cc2)s1)c1ccn(-c2ccccc2)n1] | A | N/A | B |
| 111 | 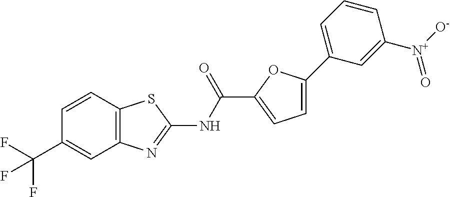 [SMILES: O=C(Nc1nc2cc(C(F)(F)F)ccc2s1)c1ccc(-c2cccc([N+](=O)[O-])c2)o1] | C | N/A | C |
| 112 | 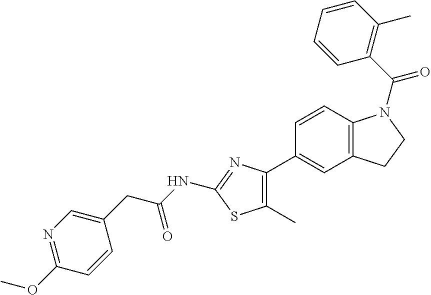 [SMILES: COc1ccc(CC(=O)Nc2nc(-c3ccc4c(c3)CCN4C(=O)c3ccccc3C)c(C)s2)cn1] | A | N/A | A |
| 113 | 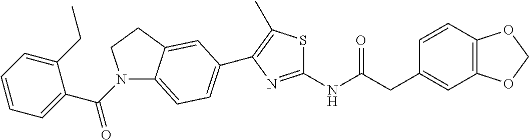 [SMILES: CCc1ccccc1C(=O)N1CCc2cc(-c3nc(NC(=O)Cc4ccc5c(c4)OCO5)sc3C)ccc21] | A | N/A | C |
| 114 | 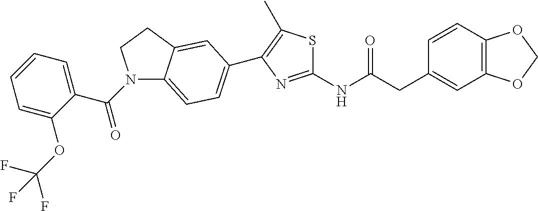 [SMILES: Cc1sc(NC(=O)Cc2ccc3c(c2)OCO3)nc1-c1ccc2c(c1)CCN2C(=O)c1ccccc1OC(F)(F)F] | A | N/A | C |
| 115 | 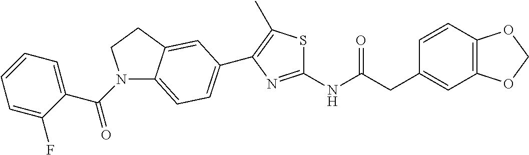 [SMILES: Cc1sc(NC(=O)Cc2ccc3c(c2)OCO3)nc1-c1ccc2c(c1)CCN2C(=O)c1ccccc1F] | A | N/A | A |
| 116 | 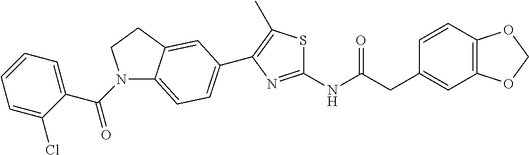 [SMILES: Cc1sc(NC(=O)Cc2ccc3c(c2)OCO3)nc1-c1ccc2c(c1)CCN2C(=O)c1ccccc1Cl] | A | N/A | C |
| 117 | 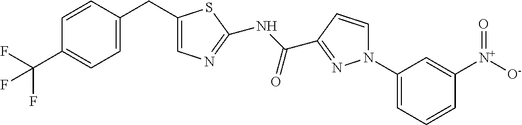 [SMILES: O=C(Nc1ncc(Cc2ccc(C(F)(F)F)cc2)s1)c1ccn(-c2cccc([N+](=O)[O-])c2)n1] | A | N/A | C |
| 118 | 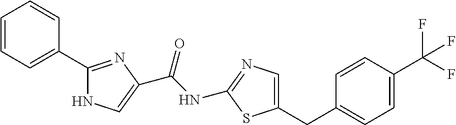 [SMILES: O=C(Nc1ncc(Cc2ccc(C(F)(F)F)cc2)s1)c1c[nH]c(-c2ccccc2)n1] | B | N/A | C |
| 119 | 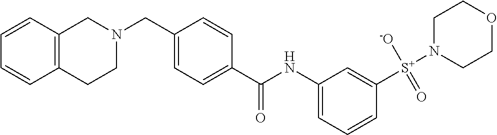 | B | N/A | C |
| 120 | 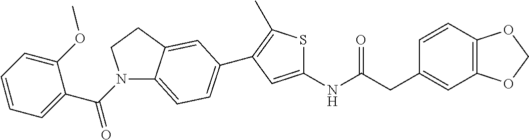 [SMILES: COc1ccccc1C(=O)N1CCc2cc(-c3cc(NC(=O)Cc4ccc5c(c4)OCO5)sc3C)ccc21] | A | N/A | C |
| 121 | 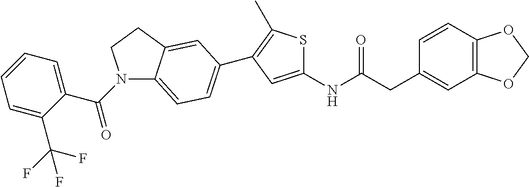 [SMILES: Cc1sc(NC(=O)Cc2ccc3c(c2)OCO3)cc1-c1ccc2c(c1)CCN2C(=O)c1ccccc1C(F)(F)F] | A | N/A | C |
| 122 | 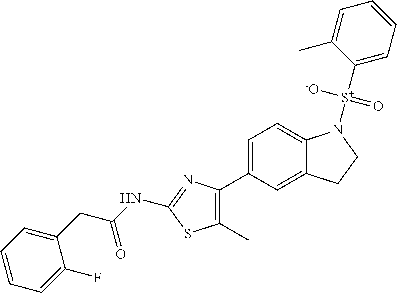 | A | N/A | A |
| 123 | 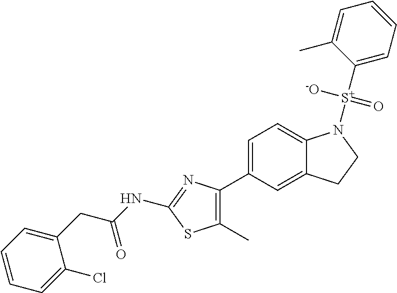 | A | N/A | C |
| 124 | 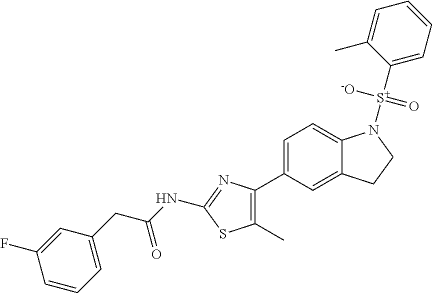 | A | N/A | A |
| 125 | 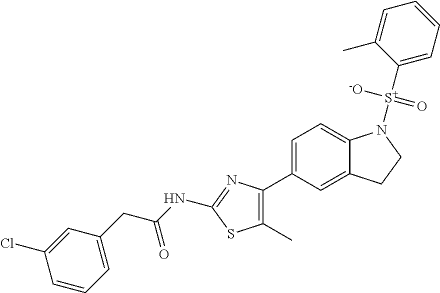 | A | N/A | B |
| 126 | 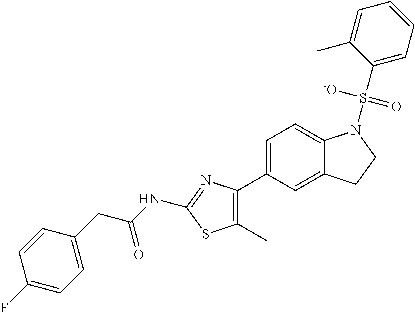 | A | N/A | C |
| 127 | 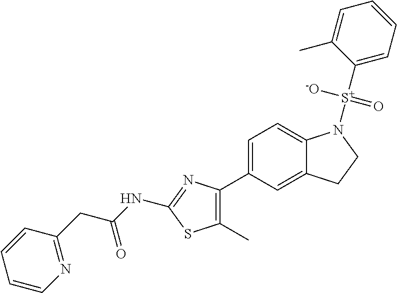 | B | N/A | B |
| 128 | 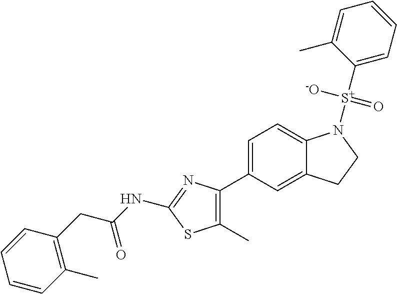 | A | N/A | A |
| 129 | 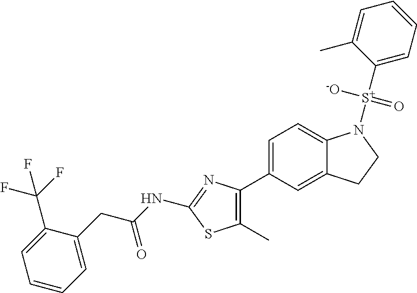 | A | N/A | C |
| 130 | 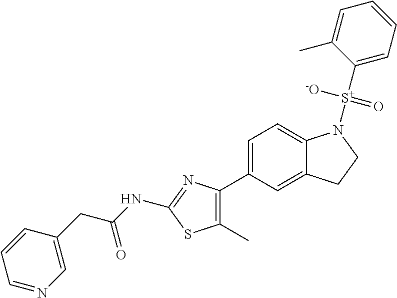 | A | N/A | A |
| 131 | 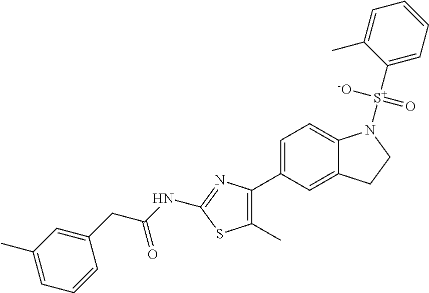 | B | N/A | B |
| 132 | 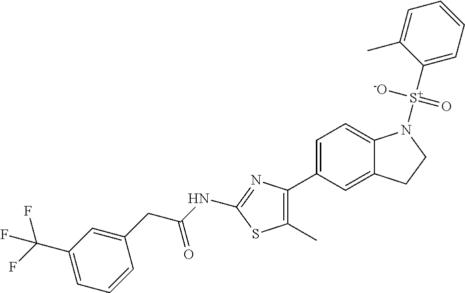 | B | N/A | B |
| 133 | 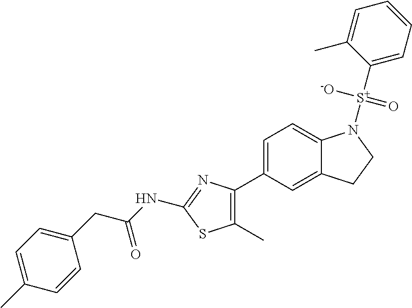 | A | N/A | C |
| 134 | 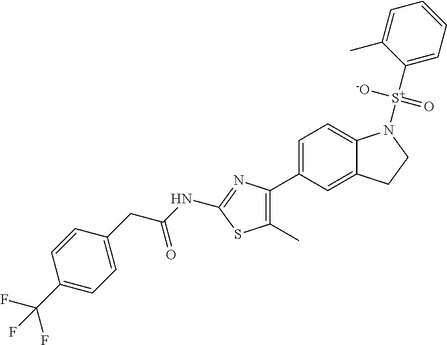 | A | N/A | C |
| 135 | 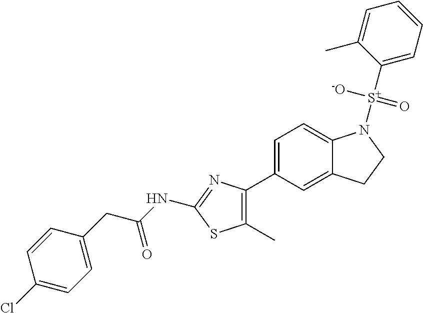 | A | N/A | B |
| 136 | 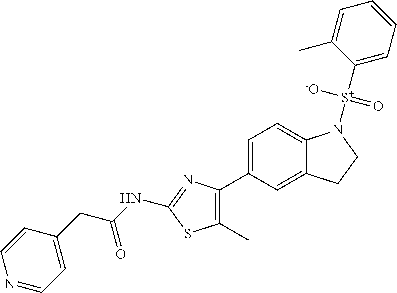 | A | N/A | A |
| 137 | 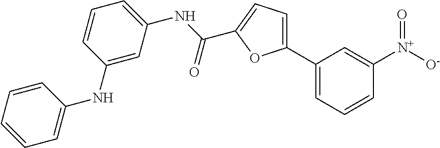 [SMILES: O=C(Nc1cccc(Nc2ccccc2)c1)c1ccc(-c2cccc([N+](=O)[O-])c2)o1] | A | N/A | C |
| 138 | 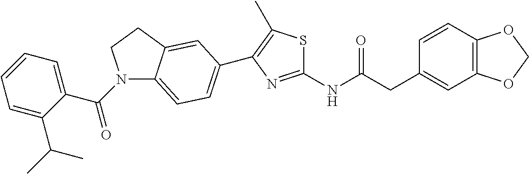 [SMILES: Cc1sc(NC(=O)Cc2ccc3c(c2)OCO3)nc1-c1ccc2c(c1)CCN2C(=O)c1ccccc1C(C)C] | A | N/A | C |
| 139 | 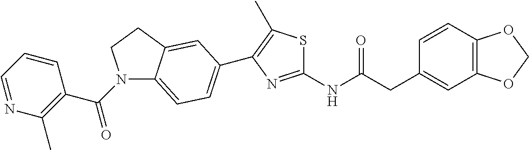 [SMILES: Cc1ncccc1C(=O)N1CCc2cc(-c3nc(NC(=O)Cc4ccc5c(c4)OCO5)sc3C)ccc21] | B | N/A | A |
| 140 | 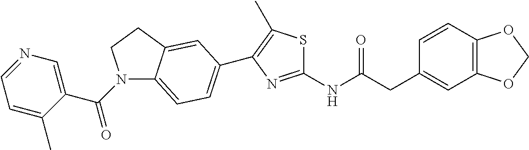 [SMILES: Cc1ccncc1C(=O)N1CCc2cc(-c3nc(NC(=O)Cc4ccc5c(c4)OCO5)sc3C)ccc21] | A | N/A | A |
| 141 | 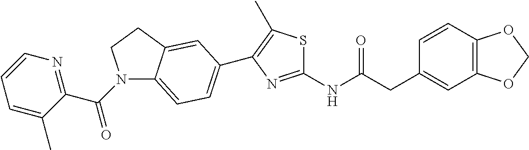 [SMILES: Cc1cccnc1C(=O)N1CCc2cc(-c3nc(NC(=O)Cc4ccc5c(c4)OCO5)sc3C)ccc21] | A | N/A | C |
| 142 | 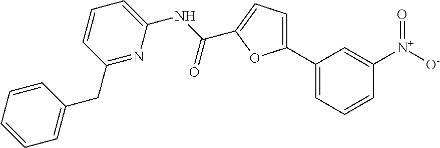 [SMILES: O=C(Nc1cccc(Cc2ccccc2)n1)c1ccc(-c2cccc([N+](=O)[O-])c2)o1] | B | N/A | C |
| 143 | 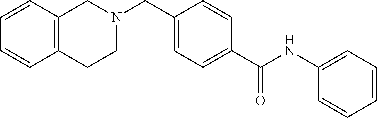 [SMILES: O=C(Nc1ccccc1)c1ccc(CN2CCc3ccccc3C2)cc1] | B | N/A | C |
| 144 | 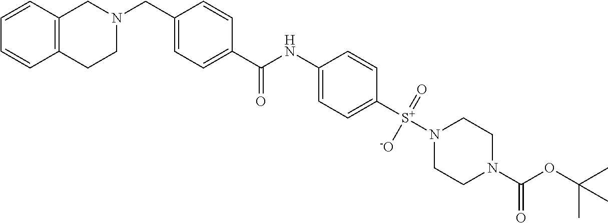 | A | N/A | A |
| 145 | 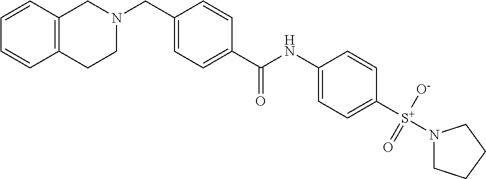 [SMILES: O=C(Nc1ccc([S+](=O)([O-])N2CCCC2)cc1)c1ccc(CN2CCc3ccccc3C2)cc1] | A | N/A | C |
| 146 | 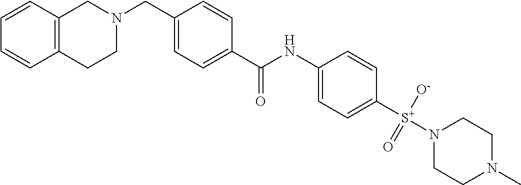 [SMILES: CN1CCN([S+](=O)([O-])c2ccc(NC(=O)c3ccc(CN4CCc5ccccc5C4)cc3)cc2)CC1] | A | N/A | C |
| 147 | 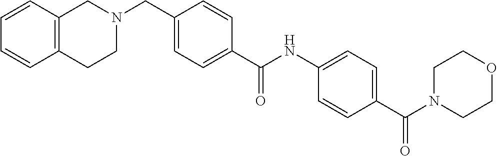 [SMILES: O=C(Nc1ccc(C(=O)N2CCOCC2)cc1)c1ccc(CN2CCc3ccccc3C2)cc1] | B | N/A | C |
| 148 | 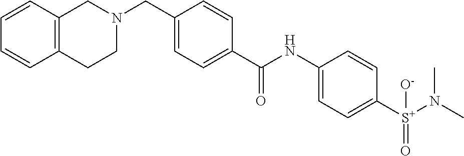 [SMILES: CN(C)[S+](=O)([O-])c1ccc(NC(=O)c2ccc(CN3CCc4ccccc4C3)cc2)cc1] | A | N/A | C |
| 149 | 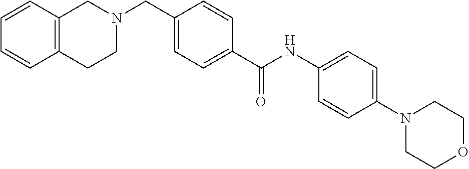 [SMILES: O=C(Nc1ccc(N2CCOCC2)cc1)c1ccc(CN2CCc3ccccc3C2)cc1] | A | N/A | C |
| 150 | 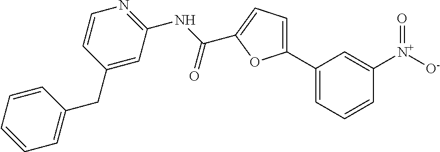 [SMILES: O=C(Nc1cc(Cc2ccccc2)ccn1)c1ccc(-c2cccc([N+](=O)[O-])c2)o1] | A | N/A | C |
| 151 | 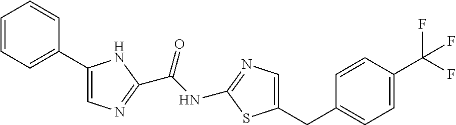 [SMILES: O=C(Nc1ncc(Cc2ccc(C(F)(F)F)cc2)s1)c1ncc(-c2ccccc2)[nH]1] | C | N/A | C |
| 152 | 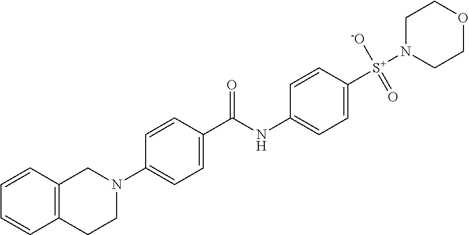 | A | N/A | A |
| 153 | 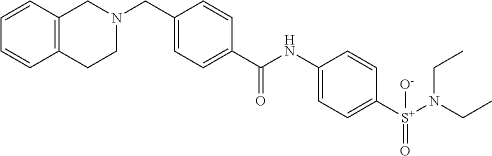 [SMILES: CCN(CC)[S+](=O)([O-])c1ccc(NC(=O)c2ccc(CN3CCc4ccccc4C3)cc2)cc1] | A | N/A | A |
| 154 | 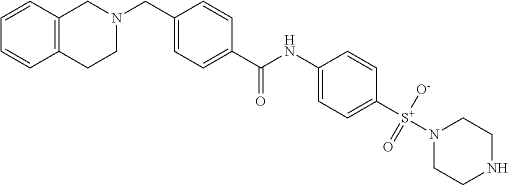 [SMILES: O=C(Nc1ccc([S+](=O)([O-])N2CCNCC2)cc1)c1ccc(CN2CCc3ccccc3C2)cc1] | B | N/A | C |
| 155 | 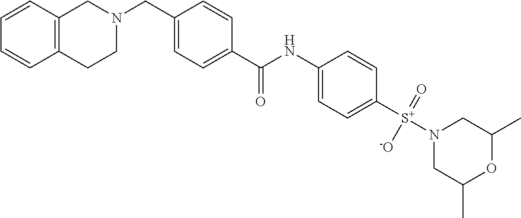 | A | N/A | A |
| 156 | 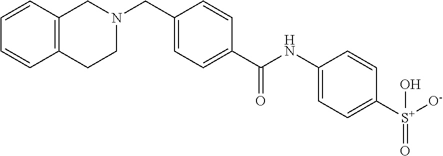 [SMILES: O=C(Nc1ccc([S+](=O)([O-])O)cc1)c1ccc(CN2CCc3ccccc3C2)cc1] | C | N/A | C |
| 157 | 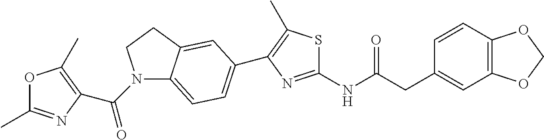 [SMILES: Cc1nc(C(=O)N2CCc3cc(-c4nc(NC(=O)Cc5ccc6c(c5)OCO6)sc4C)ccc32)c(C)o1] | A | N/A | A |
| 158 | 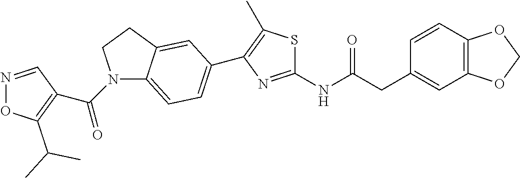 [SMILES: Cc1sc(NC(=O)Cc2ccc3c(c2)OCO3)nc1-c1ccc2c(c1)CCN2C(=O)c1cnoc1C(C)C] | A | N/A | C |
| 159 | 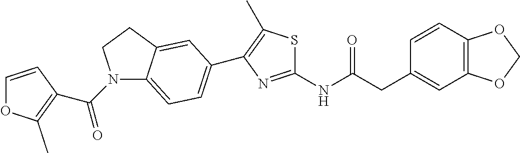 [SMILES: Cc1occc1C(=O)N1CCc2cc(-c3nc(NC(=O)Cc4ccc5c(c4)OCO5)sc3C)ccc21] | A | N/A | A |
| 160 | 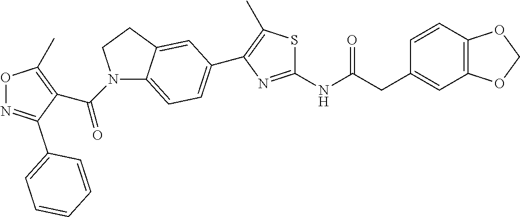 [SMILES: Cc1onc(-c2ccccc2)c1C(=O)N1CCc2cc(-c3nc(NC(=O)Cc4ccc5c(c4)OCO5)sc3C)ccc21] | C | N/A | C |
| 161 | 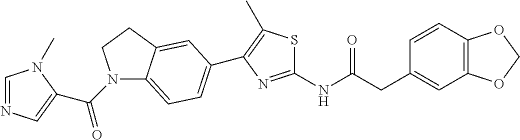 [SMILES: Cc1sc(NC(=O)Cc2ccc3c(c2)OCO3)nc1-c1ccc2c(c1)CCN2C(=O)c1cncn1C] | A | N/A | A |
| 162 | 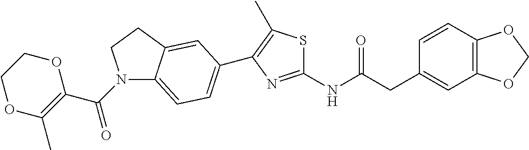 [SMILES: CC1=C(C(=O)N2CCc3cc(-c4nc(NC(=O)Cc5ccc6c(c5)OCO6)sc4C)ccc32)OCCO1] | A | N/A | A |
| 163 | 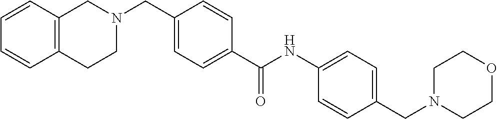 [SMILES: O=C(Nc1ccc(CN2CCOCC2)cc1)c1ccc(CN2CCc3ccccc3C2)cc1] | A | N/A | C |
| 164 | 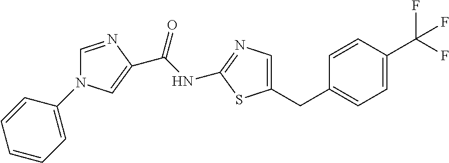 [SMILES: O=C(Nc1ncc(Cc2ccc(C(F)(F)F)cc2)s1)c1cn(-c2ccccc2)cn1] | A | N/A | C |
| 165 | 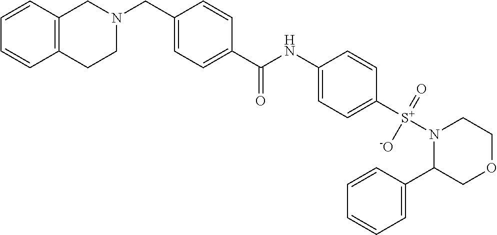 | A | N/A | B |
| 166 | 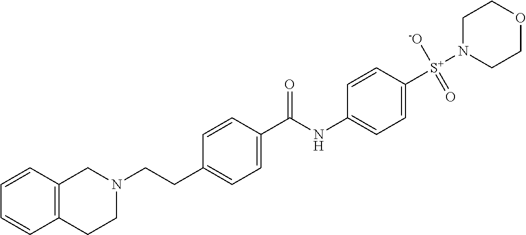 | A | N/A | B |
| 167 | 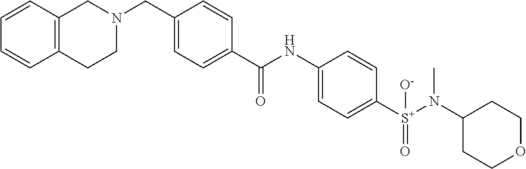 [SMILES: CN(C1CCOCC1)[S+](=O)([O-])c1ccc(NC(=O)c2ccc(CN3CCc4ccccc4C3)cc2)cc1] | A | N/A | B |
| 168 | 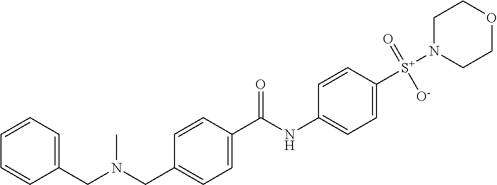 [SMILES: CN(Cc1ccccc1)Cc1ccc(C(=O)Nc2ccc([S+](=O)([O-])N3CCOCC3)cc2)cc1] | A | N/A | A |
| 169 | 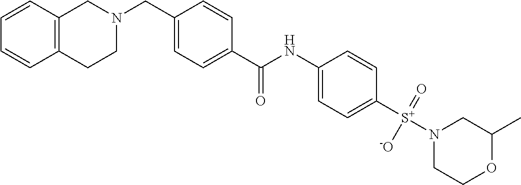 | A | N/A | A |
| 170 | 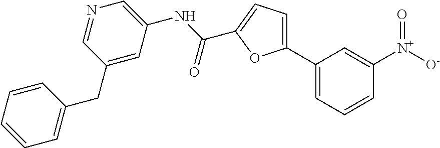 [SMILES: O=C(Nc1cncc(Cc2ccccc2)c1)c1ccc(-c2cccc([N+](=O)[O-])c2)o1] | B | N/A | B |
| 171 | 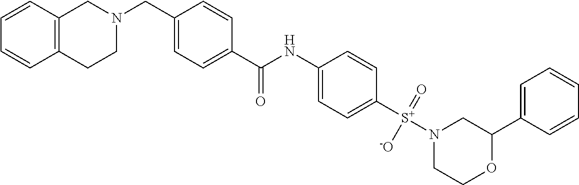 | A | N/A | C |
| 172 | 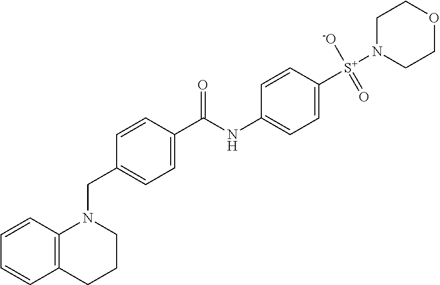 | A | N/A | A |
| 173 | 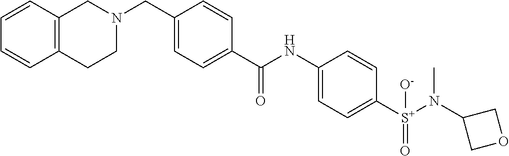 [SMILES: CN(C1COC1)[S+](=O)([O-])c1ccc(NC(=O)c2ccc(CN3CCc4ccccc4C3)cc2)cc1] | A | N/A | A |
| 174 | 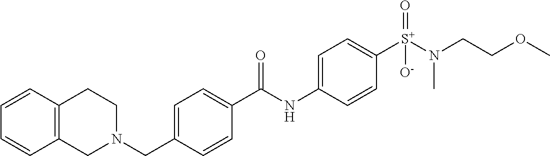 [SMILES: COCCN(C)[S+](=O)([O-])c1ccc(NC(=O)c2ccc(CN3CCc4ccccc4C3)cc2)cc1] | A | N/A | C |
| 175 | 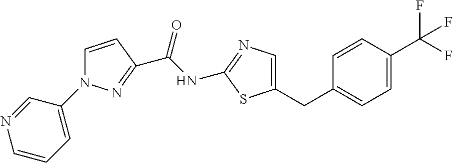 [SMILES: O=C(Nc1ncc(Cc2ccc(C(F)(F)F)cc2)s1)c1ccn(-c2cccnc2)n1] | B | N/A | B |
| 176 | 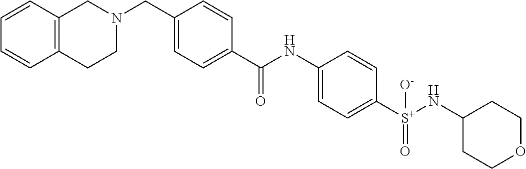 [SMILES: O=C(Nc1ccc([S+](=O)([O-])NC2CCOCC2)cc1)c1ccc(CN2CCc3ccccc3C2)cc1] | A | N/A | B |
| 177 | 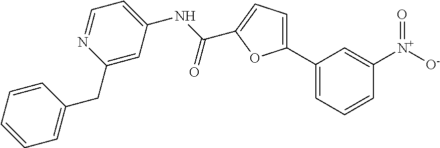 [SMILES: O=C(Nc1ccnc(Cc2ccccc2)c1)c1ccc(-c2cccc([N+](=O)[O-])c2)o1] | B | N/A | B |
| 178 | 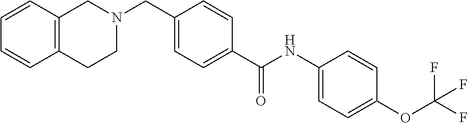 [SMILES: O=C(Nc1ccc(OC(F)(F)F)cc1)c1ccc(CN2CCc3ccccc3C2)cc1] | A | N/A | A |
| 179 | 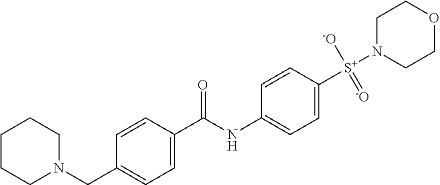 | B | N/A | C |
| 180 | 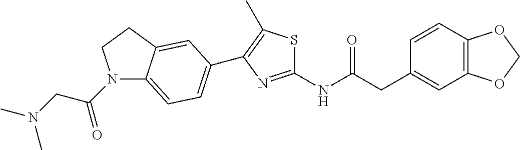 [SMILES: Cc1sc(NC(=O)Cc2ccc3c(c2)OCO3)nc1-c1ccc2c(c1)CCN2C(=O)CN(C)C] | B | N/A | C |
| 181 | 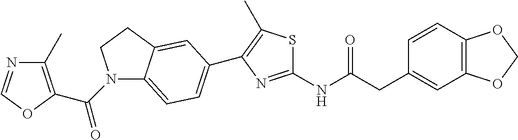 [SMILES: Cc1ncoc1C(=O)N1CCc2cc(-c3nc(NC(=O)Cc4ccc5c(c4)OCO5)sc3C)ccc21] | A | N/A | C |
| 182 | 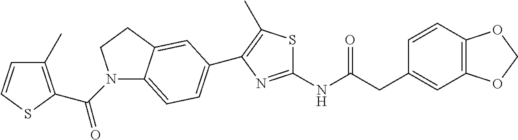 [SMILES: Cc1ccsc1C(=O)N1CCc2cc(-c3nc(NC(=O)Cc4ccc5c(c4)OCO5)sc3C)ccc21] | A | N/A | A |
| 183 | 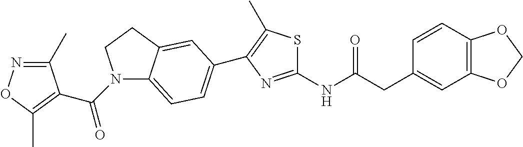 [SMILES: Cc1noc(C)c1C(=O)N1CCc2cc(-c3nc(NC(=O)Cc4ccc5c(c4)OCO5)sc3C)ccc21] | A | N/A | A |
| 184 | 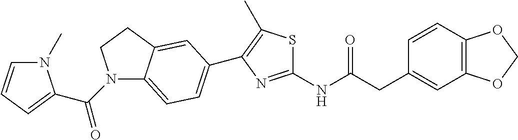 [SMILES: Cc1sc(NC(=O)Cc2ccc3c(c2)OCO3)nc1-c1ccc2c(c1)CCN2C(=O)c1cccn1C] | A | N/A | A |
| 185 | 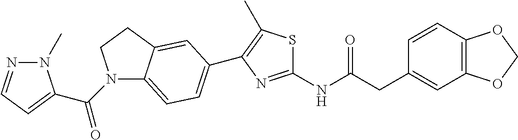 [SMILES: Cc1sc(NC(=O)Cc2ccc3c(c2)OCO3)nc1-c1ccc2c(c1)CCN2C(=O)c1ccnn1C] | A | N/A | A |
| 186 | 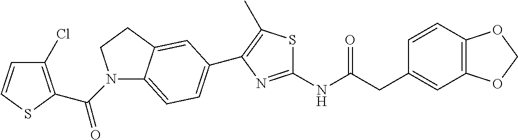 [SMILES: Cc1sc(NC(=O)Cc2ccc3c(c2)OCO3)nc1-c1ccc2c(c1)CCN2C(=O)c1sccc1Cl] | A | N/A | A |
| 187 | 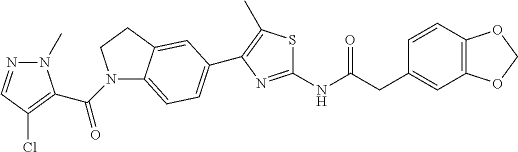 [SMILES: Cc1sc(NC(=O)Cc2ccc3c(c2)OCO3)nc1-c1ccc2c(c1)CCN2C(=O)c1c(Cl)cnn1C] | A | N/A | A |
| 188 | 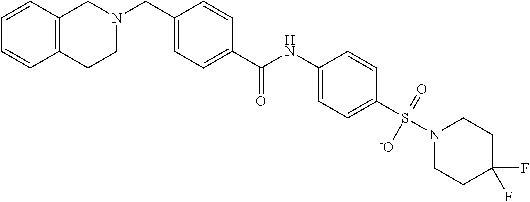 | A | N/A | A |
| 189 | 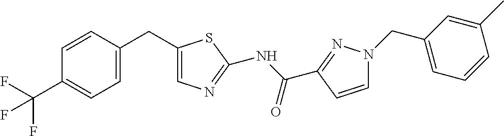 [SMILES: Cc1cccc(Cn2ccc(C(=O)Nc3ncc(Cc4ccc(C(F)(F)F)cc4)s3)n2)c1] | B | N/A | A |
| 190 | 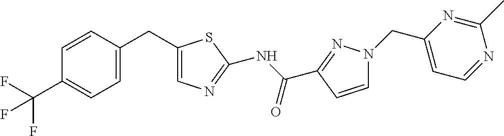 [SMILES: Cc1nccc(Cn2ccc(C(=O)Nc3ncc(Cc4ccc(C(F)(F)F)cc4)s3)n2)n1] | B | N/A | B |
| 191 | 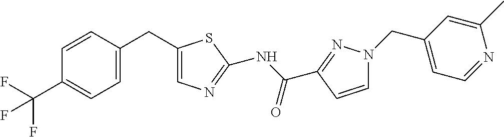 [SMILES: Cc1cc(Cn2ccc(C(=O)Nc3ncc(Cc4ccc(C(F)(F)F)cc4)s3)n2)ccn1] | A | N/A | A |
| 192 | 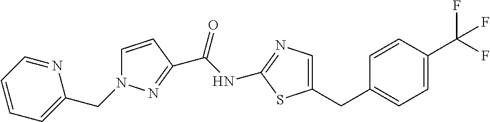 [SMILES: O=C(Nc1ncc(Cc2ccc(C(F)(F)F)cc2)s1)c1ccn(Cc2ccccn2)n1] | C | N/A | C |
| 193 | 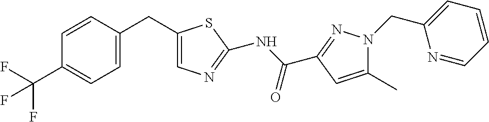 [SMILES: Cc1cc(C(=O)Nc2ncc(Cc3ccc(C(F)(F)F)cc3)s2)nn1Cc1ccccn1] | A | N/A | A |
| 194 | 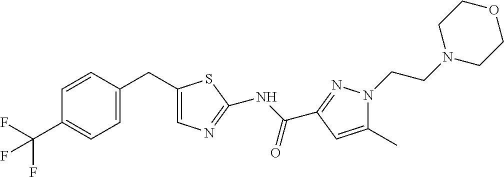 [SMILES: Cc1cc(C(=O)Nc2ncc(Cc3ccc(C(F)(F)F)cc3)s2)nn1CCN1CCOCC1] | B | N/A | B |
| 195 | 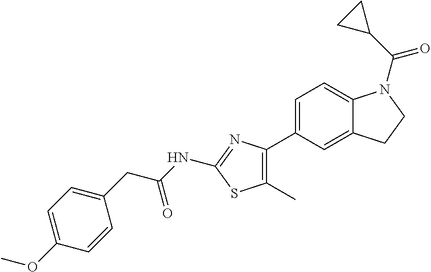 [SMILES: COc1ccc(CC(=O)Nc2nc(-c3ccc4c(c3)CCN4C(=O)C3CC3)c(C)s2)cc1] | A | N/A | B |
| 196 | 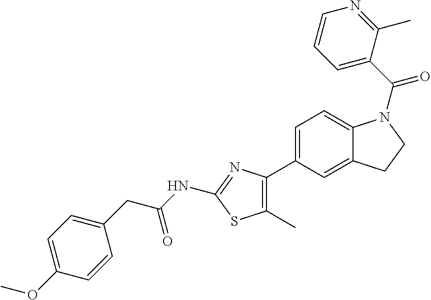 [SMILES: COc1ccc(CC(=O)Nc2nc(-c3ccc4c(c3)CCN4C(=O)c3cccnc3C)c(C)s2)cc1] | A | N/A | A |
| 197 | 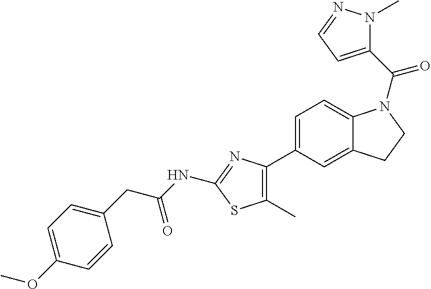 [SMILES: COc1ccc(CC(=O)Nc2nc(-c3ccc4c(c3)CCN4C(=O)c3ccnn3C)c(C)s2)cc1] | A | N/A | A |
| 198 | 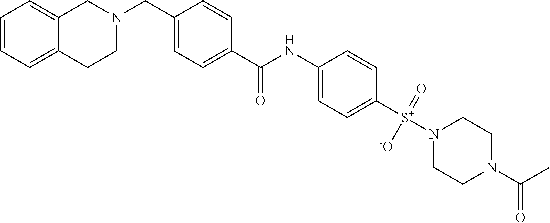 | A | N/A | A |
| 199 | 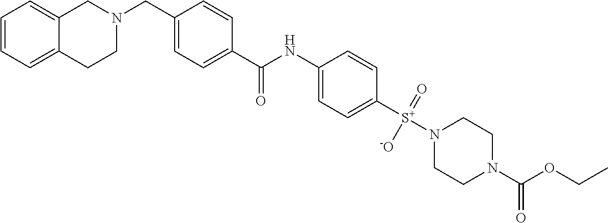 | A | N/A | A |
| 200 | 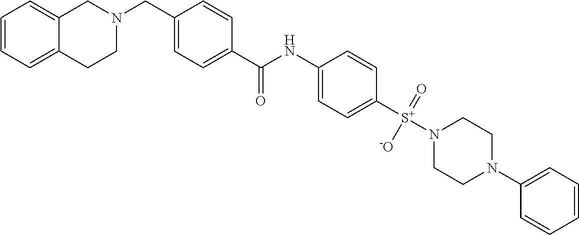 | A | N/A | A |
| 201 | 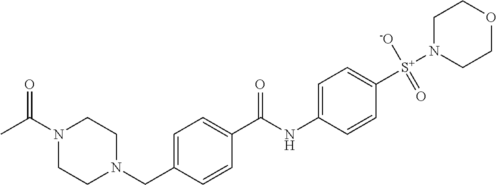 | B | N/A | B |
| 202 | 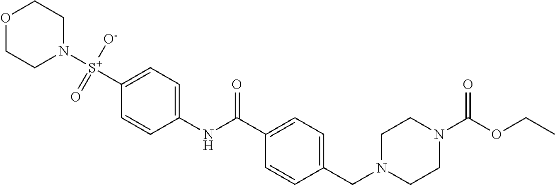 [SMILES: CCOC(=O)N1CCN(Cc2ccc(C(=O)Nc3ccc([S+](=O)([O-])N4CCOCC4)cc3)cc2)CC1] | B | N/A | B |
| 203 | 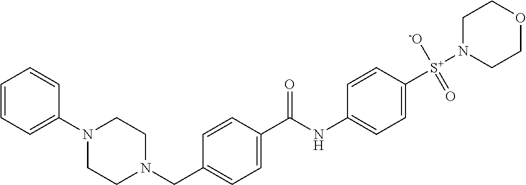 | A | N/A | A |
| 204 | 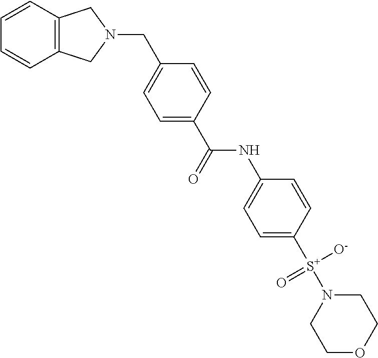 [SMILES: O=C(Nc1ccc([S+](=O)([O-])N2CCOCC2)cc1)c1ccc(CN2Cc3ccccc3C2)cc1] | A | N/A | A |
| 205 | 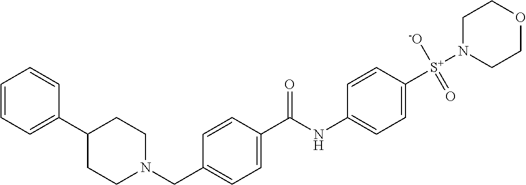 | A | N/A | A |
| 206 | 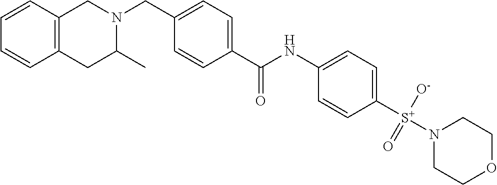 [SMILES: CC1Cc2ccccc2CN1Cc1ccc(C(=O)Nc2ccc([S+](=O)([O-])N3CCOCC3)cc2)cc1] | A | N/A | A |
| 207 | 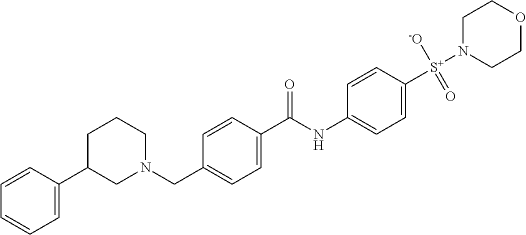 | B | N/A | B |
| 208 | 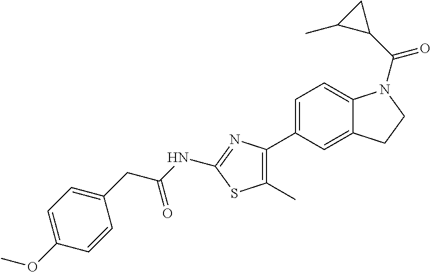 [SMILES: COc1ccc(CC(=O)Nc2nc(-c3ccc4c(c3)CCN4C(=O)C3CC3C)c(C)s2)cc1] | A | N/A | A |
| 209 | 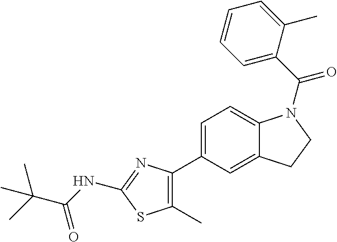 [SMILES: Cc1ccccc1C(=O)N1CCc2cc(-c3nc(NC(=O)C(C)(C)C)sc3C)ccc21] | B | N/A | A |
| 210 | 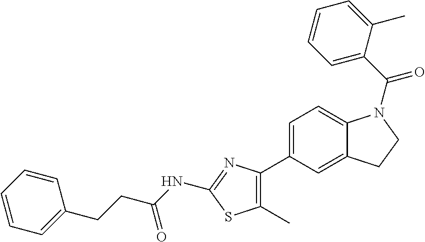 [SMILES: Cc1ccccc1C(=O)N1CCc2cc(-c3nc(NC(=O)CCc4ccccc4)sc3C)ccc21] | A | N/A | B |
| 211 | 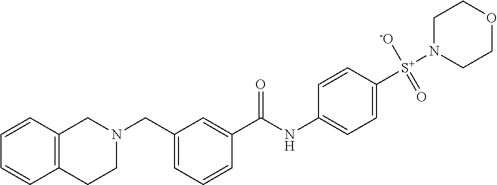 | B | N/A | B |
| 212 | 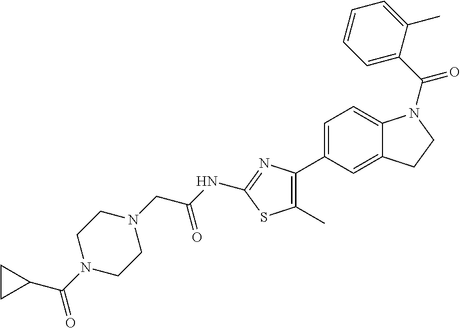 [SMILES: Cc1ccccc1C(=O)N1CCc2cc(-c3nc(NC(=O)CN4CCN(C(=O)C5CC5)CC4)sc3C)ccc21] | A | N/A | A |
| 213 | 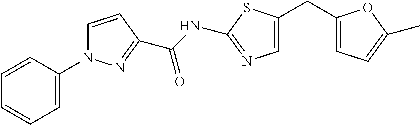 [SMILES: Cc1ccc(Cc2cnc(NC(=O)c3ccn(-c4ccccc4)n3)s2)o1] | B | N/A | B |
| 214 | 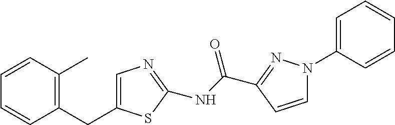 [SMILES: Cc1ccccc1Cc1cnc(NC(=O)c2ccn(-c3ccccc3)n2)s1] | B | N/A | B |
| 215 | 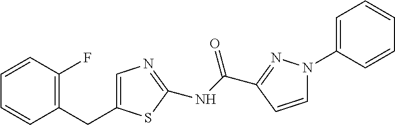 [SMILES: O=C(Nc1ncc(Cc2ccccc2F)s1)c1ccn(-c2ccccc2)n1] | A | N/A | A |
| 216 | 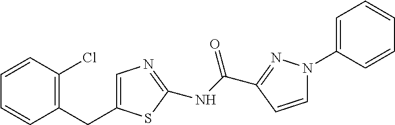 [SMILES: O=C(Nc1ncc(Cc2ccccc2Cl)s1)c1ccn(-c2ccccc2)n1] | B | N/A | B |
| 217 | 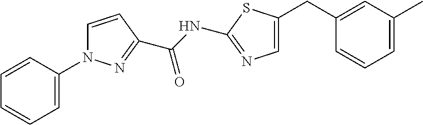 [SMILES: Cc1cccc(Cc2cnc(NC(=O)c3ccn(-c4ccccc4)n3)s2)c1] | B | N/A | B |
| 218 | 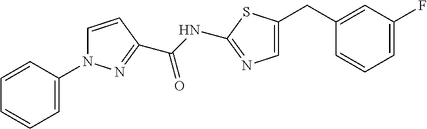 [SMILES: O=C(Nc1ncc(Cc2cccc(F)c2)s1)c1ccn(-c2ccccc2)n1] | B | N/A | A |
| 219 | 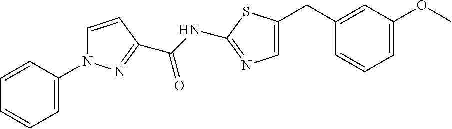 [SMILES: COc1cccc(Cc2cnc(NC(=O)c3ccn(-c4ccccc4)n3)s2)c1] | A | N/A | B |
| 220 | 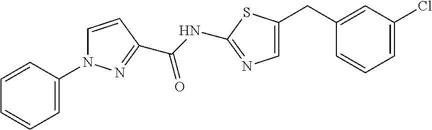 [SMILES: O=C(Nc1ncc(Cc2cccc(Cl)c2)s1)c1ccn(-c2ccccc2)n1] | B | N/A | A |
| 221 | 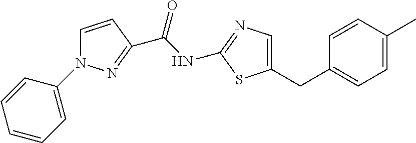 [SMILES: Cc1ccc(Cc2cnc(NC(=O)c3ccn(-c4ccccc4)n3)s2)cc1] | B | N/A | B |
| 222 | 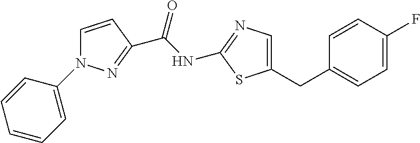 [SMILES: O=C(Nc1ncc(Cc2ccc(F)cc2)s1)c1ccn(-c2ccccc2)n1] | A | N/A | A |
| 223 | 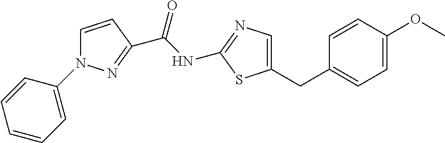 [SMILES: COc1ccc(Cc2cnc(NC(=O)c3ccn(-c4ccccc4)n3)s2)cc1] | B | N/A | B |
| 224 | 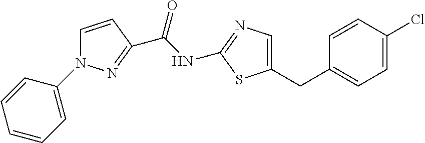 [SMILES: O=C(Nc1ncc(Cc2ccc(Cl)cc2)s1)c1ccn(-c2ccccc2)n1] | A | N/A | A |
| 225 | 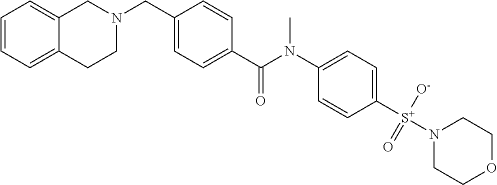 [SMILES: CN(C(=O)c1ccc(CN2CCc3ccccc3C2)cc1)c1ccc([S+](=O)([O-])N2CCOCC2)cc1] | B | N/A | C |
| 226 | 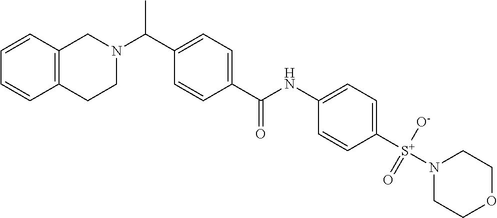 [SMILES: CC(c1ccc(C(=O)Nc2ccc([S+](=O)([O-])N3CCOCC3)cc2)cc1)N1CCc2ccccc2C1] | A | N/A | A |
| 227 | 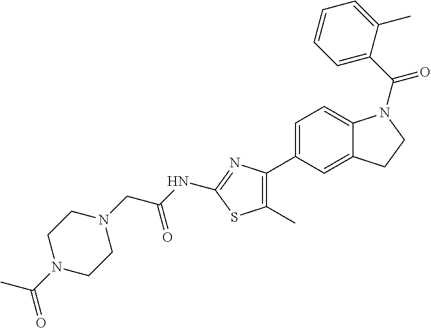 [SMILES: CC(=O)N1CCN(CC(=O)Nc2nc(-c3ccc4c(c3)CCN4C(=O)c3ccccc3C)c(C)s2)CC1] | B | N/A | B |
| 228 | 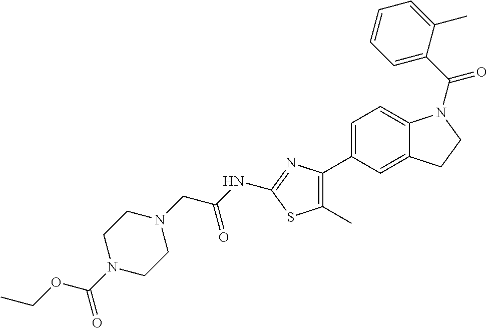 [SMILES: CCOC(=O)N1CCN(CC(=O)Nc2nc(-c3ccc4c(c3)CCN4C(=O)c3ccccc3C)c(C)s2)CC1] | A | N/A | A |
| 229 | 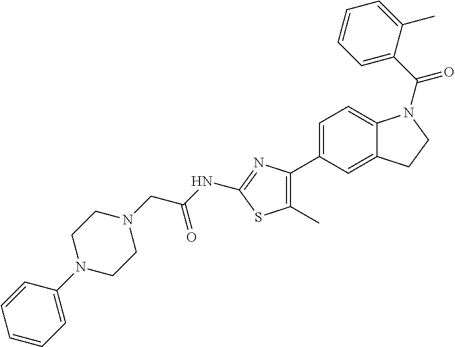 [SMILES: Cc1ccccc1C(=O)N1CCc2cc(-c3nc(NC(=O)CN4CCN(c5ccccc5)CC4)sc3C)ccc21] | A | N/A | A |
| 230 | 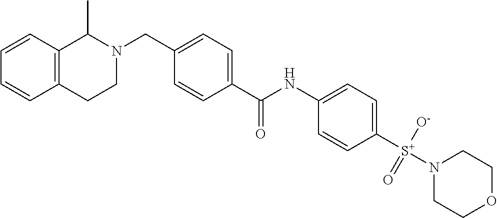 [SMILES: CC1c2ccccc2CCN1Cc1ccc(C(=O)Nc2ccc([S+](=O)([O-])N3CCOCC3)cc2)cc1] | A | N/A | A |
| 231 | 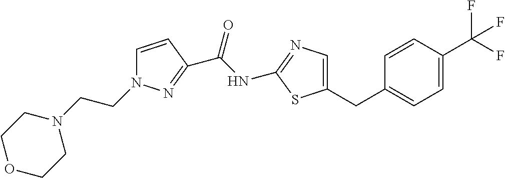 [SMILES: O=C(Nc1ncc(Cc2ccc(C(F)(F)F)cc2)s1)c1ccn(CCN2CCOCC2)n1] | B | N/A | A |
| 232 | 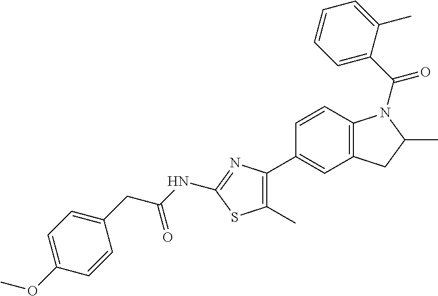 | B | N/A | B |
| 233 | 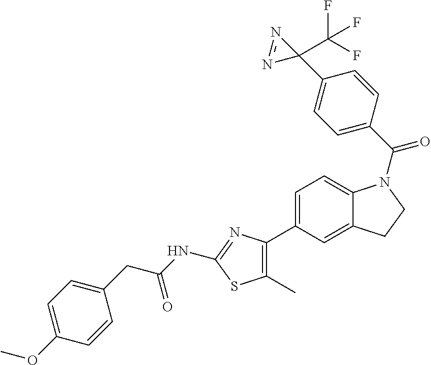 [SMILES: COc1ccc(CC(=O)Nc2nc(-c3ccc4c(c3)CCN4C(=O)c3ccc(C4(C(F)(F)F)N=N4)cc3)c(C)s2)cc1] | B | N/A | B |
| 234 | 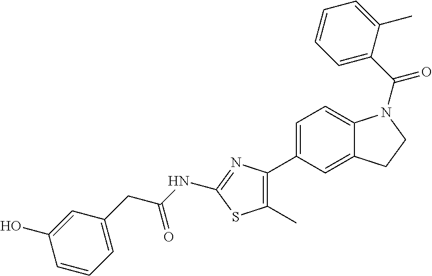 [SMILES: Cc1ccccc1C(=O)N1CCc2cc(-c3nc(NC(=O)Cc4cccc(O)c4)sc3C)ccc21] | B | N/A | B |
| 235 | 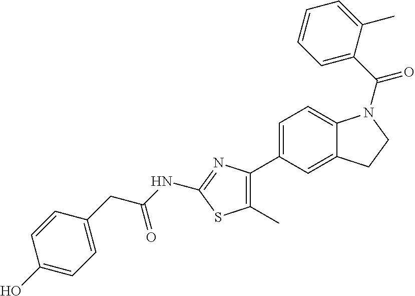 [SMILES: Cc1ccccc1C(=O)N1CCc2cc(-c3nc(NC(=O)Cc4ccc(O)cc4)sc3C)ccc21] | B | N/A | B |
| 236 | 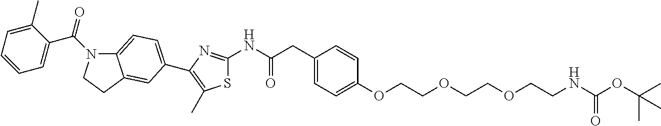 [SMILES: Cc1ccccc1C(=O)N1CCc2cc(-c3nc(NC(=O)Cc4ccc(OCCOCCOCCNC(=O)OC(C)(C)C)cc4)sc3C)ccc21] | A | N/A | A |
| 237 | 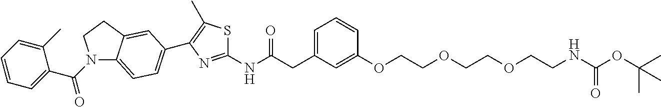 [SMILES: Cc1ccccc1C(=O)N1CCc2cc(-c3nc(NC(=O)Cc4cccc(OCCOCCOCCNC(=O)OC(C)(C)C)c4)sc3C)ccc21] | A | N/A | A |
| 238 | 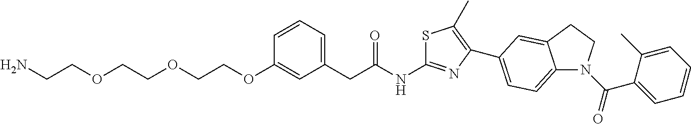 [SMILES: Cc1ccccc1C(=O)N1CCc2cc(-c3nc(NC(=O)Cc4cccc(OCCOCCOCCN)c4)sc3C)ccc21] | B | N/A | B |
| 239 | 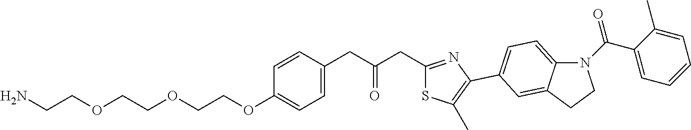 [SMILES: Cc1ccccc1C(=O)N1CCc2cc(-c3nc(CC(=O)Cc4ccc(OCCOCCOCCN)cc4)sc3C)ccc21] | C | N/A | B |
| 240 | 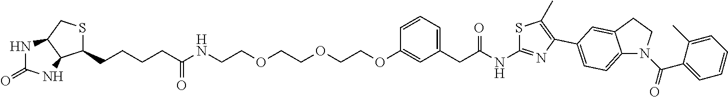 [SMILES: Cc1ccccc1C(=O)N1CCc2cc(-c3nc(NC(=O)Cc4cccc(OCCOCCOCCNC(=O)CCCC[C@@H]5SC[C@@H]6NC(=O)N[C@@H]65)c4)sc3C)ccc21] | B | N/A | A |
| 241 | 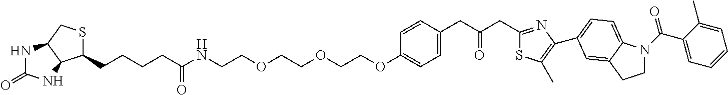 [SMILES: Cc1ccccc1C(=O)N1CCc2cc(-c3nc(CC(=O)Cc4ccc(OCCOCCOCCNC(=O)CCCC[C@@H]5SC[C@@H]6NC(=O)N[C@@H]65)cc4)sc3C)ccc21] | C | N/A | B |

### Example 6

### Preparation of Representative Compounds

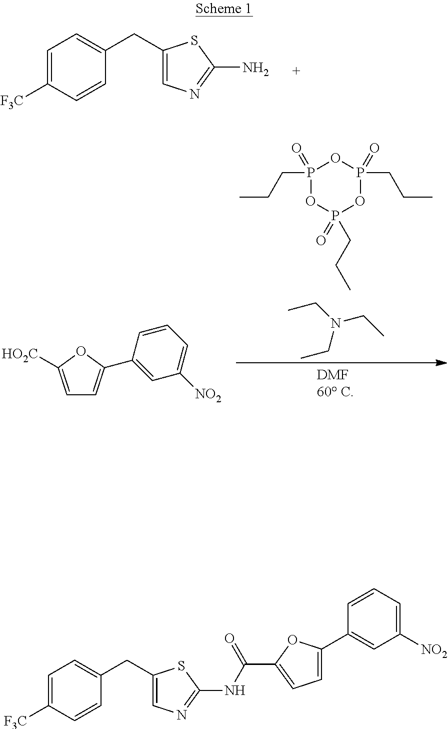

Scheme 1

To a solution of 5-(3-nitrophenyl)furan-2-carboxylic acid (423 mg, 1.812 mmol), 5-(4-(trifluoromethyl)benzyl)thiazol-2-amine (390 mg, 1.510 mmol), and triethylamine (1.052 mL, 7.55 mmol) in EtOAc (10 ml) was added 50 wt. % propylphosphonic anhydride solution in EtOAc (1.8 mL, 3.02 mmol). The mixture was stirred at rt for 10 min and then heated at 60° C. for 3 hr. The reaction mixture was cooled to rt and diluted with water and EtOAc. The layers were separated and the aqueous layer was reextracted with EtOAc. The combined organic layers were washed with water, dried with MgSO₄, and concentrated in vacuo to afford a residue. The residue was taken up in DMSO and subsequently purified by reverse phase chromatography to give Compound 1.

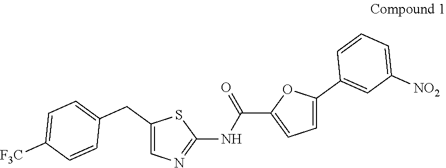 [SMILES: O=C(Nc1ncc(Cc2ccc(C(F)(F)F)cc2)s1)c1ccc(-c2cccc([N+](=O)[O-])c2)o1]

¹H NMR (400 MHz, DMSO-d6) δ ppm 12.87 (s, 1H), 8.84 (t, J=1.9 Hz, 1H), 8.51-8.44 (m, 1H), 8.26 (ddd, J=8.2, 2.3, 1.0 Hz, 1H), 7.82 (ddd, J=8.3, 7.9, 0.4 Hz, 1H), 7.73 (dq, J=8.0, 0.7 Hz, 2H), 7.65-7.49 (m, 4H), 7.45-7.40 (m, 1H), 4.28 (s, 2H); HRMS: m/z (M+H)⁺=474.0715 (Calculated for C₂₂H₁₅F₃N₃O₄S=474.0730).

Compound 15 was prepared according to the method described in Scheme 1 substituting furan-2-carboxylic acid for 5-(3-nitrophenyl)furan-2-carboxylic acid.

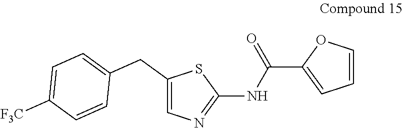 [SMILES: O=C(Nc1ncc(Cc2ccc(C(F)(F)F)cc2)s1)c1ccco1]

¹H NMR (400 MHz, DMSO-d₆) δ ppm 12.45 (s, 1H), 7.96 (dd, J=1.8, 0.8 Hz, 1H), 7.72-7.63 (m, 2H), 7.58 (s, 1H), 7.54-7.47 (m, 2H), 7.36-7.31 (m, 1H), 6.69 (dd, J=3.6, 1.7 Hz, 1H), 4.21 (s, 2H); HRMS: m/z (M+H)⁺=353.0564 (Calculated for C₁₆H₁₂F₃N₂O₂S=353.0566).

Compound 22 was prepared according to the method described in Scheme 1 substituting 5-(4-nitrophenyl)furan-2-carboxylic acid for 5-(3-nitrophenyl)furan-2-carboxylic acid.

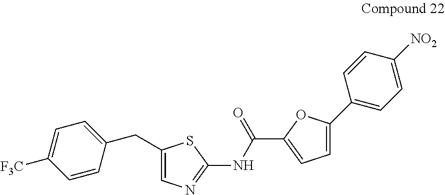 [SMILES: O=C(Nc1ncc(Cc2ccc(C(F)(F)F)cc2)s1)c1ccc(-c2ccc([N+](=O)[O-])cc2)o1]

¹H NMR (400 MHz, DMSO-d6) δ ppm 12.86 (s, 1H), 8.38-8.23 (m, 4H), 7.69 (dq, J=7.6, 0.8 Hz, 2H), 7.60 (d, J=3.7 Hz, 1H), 7.56-7.47 (m, 3H), 7.39 (d, J=1.0 Hz, 1H), 4.23 (s, 2H); HRMS: m/z (M+H)⁺=474.0727 (Calculated for C₂₂H₁₅F₃N₃O₄S=474.0730).

Compound 18 was prepared according to the method described in Scheme 1 substituting 5-(2-nitrophenyl)furan-2-carboxylic acid for 5-(3-nitrophenyl)furan-2-carboxylic acid.

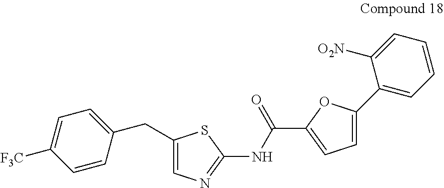 [SMILES: O=C(Nc1ncc(Cc2ccc(C(F)(F)F)cc2)s1)c1ccc(-c2ccccc2[N+](=O)[O-])o1]

¹H NMR (400 MHz, DMSO-d6) δ ppm 12.65 (s, 1H), 8.05-7.95 (m, 2H), 7.81 (td, J=7.7, 1.3 Hz, 1H), 7.72-7.62 (m, 4H), 7.55-7.47 (m, 2H), 7.36 (d, J=1.0 Hz, 1H), 6.99 (d, J=3.7 Hz, 1H), 4.22 (s, 2H); HRMS: m/z (M+H)⁺=474.0716 (Calculated for C₂₂H₁₅F₃N₃O₄S=474.0730).

Compound 19 was prepared according to the method described in Scheme 1 substituting 5-phenylfuran-2-carboxylic acid for 5-(3-nitrophenyl)furan-2-carboxylic acid.

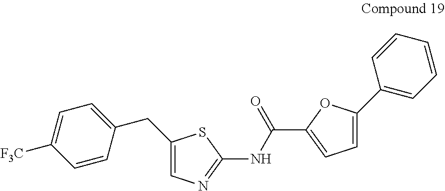 [SMILES: O=C(Nc1ncc(Cc2ccc(C(F)(F)F)cc2)s1)c1ccc(-c2ccccc2)o1]

¹H NMR (400 MHz, DMSO-d6) δ ppm 12.66 (s, 1H), 8.02-7.94 (m, 2H), 7.72-7.64 (m, 2H), 7.60-7.32 (m, 7H), 7.18 (d, J=3.7 Hz, 1H), 4.23 (s, 2H); HRMS: m/z (M+H)⁺=429.0890 (Calculated for C₂₂H₁₆F₃N₂O₂S=429.0879).

Compound 20 was prepared according to the method described in Scheme 1 substituting 5-(3-(trifluoromethyl)benzyl)thiazol-2-amine for 5-(4-(trifluoromethyl)benzyl)thiazol-2-amine.

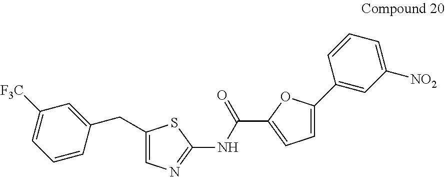 [SMILES: O=C(Nc1ncc(Cc2cccc(C(F)(F)F)c2)s1)c1ccc(-c2cccc([N+](=O)[O-])c2)o1]

¹H NMR (400 MHz, DMSO-d6) δ ppm 12.82 (s, 1H), 8.80 (t, J=1.9 Hz, 1H), 8.47-8.39 (m, 1H), 8.22 (ddd, J=8.2, 2.3, 1.0 Hz, 1H), 7.82-7.73 (m, 1H), 7.70-7.50 (m, 5H), 7.47 (d, J=3.7 Hz, 1H), 7.41-7.36 (m, 1H), 4.24 (s, 2H); HRMS: m/z (M+H)⁺=474.0720 (Calculated for C₂₂H₁₅F₃N₃O₄S=474.0730).

Compound 16 was prepared according to the method described in Scheme 1 substituting 5-benzylthiazol-2-amine for 5-(4-(trifluoromethyl)benzyl)thiazol-2-amine.

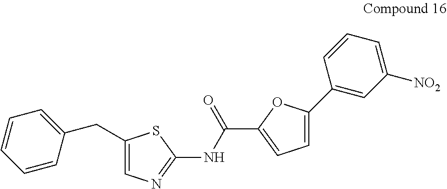 [SMILES: O=C(Nc1ncc(Cc2ccccc2)s1)c1ccc(-c2cccc([N+](=O)[O-])c2)o1]

¹H NMR (400 MHz, DMSO-d6) δ ppm 12.78 (s, 1H), 8.80 (s, 1H), 8.43 (d, J=7.9 Hz, 1H), 8.22 (ddd, J=8.2, 2.3, 1.0 Hz, 1H), 7.82-7.73 (m, 1H), 7.60-7.55 (m, 1H), 7.47 (d, J=3.7 Hz, 1H), 7.37-7.17 (m, 6H), 4.11 (s, 2H); HRMS: m/z (M+H)⁺=406.0852 (Calculated for C₂₁H₁₆N₃O₄S=406.0856).

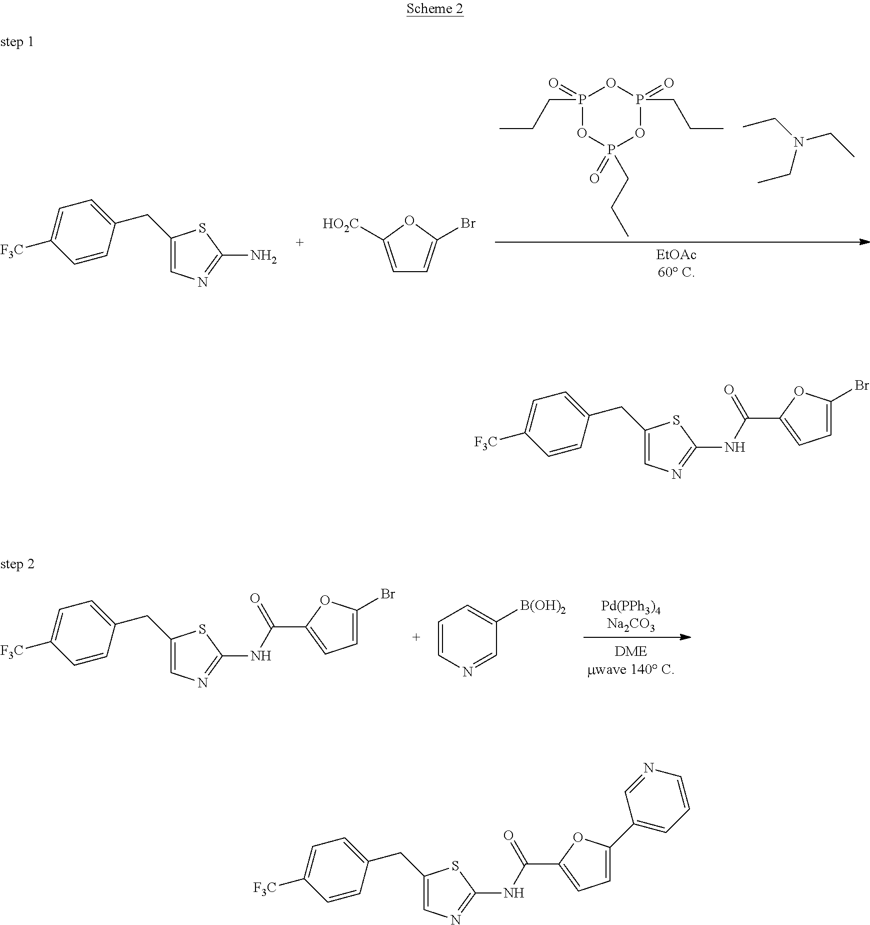

### Scheme 2, Step 1

To a solution of 5-bromofuran-2-carboxylic acid (62.1 mg, 0.325 mmol), 5-(4-(trifluoromethyl)benzyl)thiazol-2-amine (70 mg, 0.271 mmol), and triethylamine (0.189 mL, 1.355 mmol) in EtOAc (10 ml) was added 50 wt. % propylphosphonic anhydride solution in EtOAc (0.277 mL, 0.542 mmol). The mixture was stirred at rt for 10 min and then heated at 60° C. for 6 hr. The reaction mixture was cooled to rt and diluted with water and EtOAc. The layers were separated and the aqueous layer was reextracted with EtOAc. The combined organic layers were washed with water, dried with MgSO₄, and concentrated in vacuo to afford a residue. The residue was taken up in DMSO and subsequently purified by reverse phase chromatography utilizing a gradient of 10 to 100% acetonitrile/water to give 5-bromo-N-(5-(4-(trifluoromethyl)benzyl)thiazol-2-yl)furan-2-carboxamide.

### Scheme 2, Step 2

A mixture of 5-bromo-N-(5-(4-(trifluoromethyl)benzyl)thiazol-2-yl)furan-2-carboxamide (50 mg, 0.116 mmol), pyridin-3-ylboronic acid (35.6 mg, 0.290 mmol), tetrakis(triphenylphosphine)palladium(0) (13.4 mg, 0.012 mmol), and sodium carbonate (174 μL of a 2M aqueous solution, 0.348 mmol) in DME (1 ml) was heated with stirring in the microwave at 140° C. for 1 hr. The reaction mixture was concentrated under a stream of air. The residue was taken up in EtOAc, dried with MgSO₄, and filtered through an Agilent PL-Thiol MP SPE cartridge to remove palladium. The organic layer was concentrated under a stream of air. The residue was taken up in DMSO and subsequently purified by reverse phase chromatography to give Compound 27.

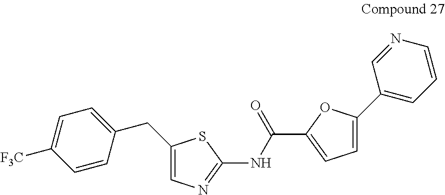 [SMILES: O=C(Nc1ncc(Cc2ccc(C(F)(F)F)cc2)s1)c1ccc(-c2cccnc2)o1]

¹H NMR (400 MHz, DMSO-d6) δ ppm 12.74 (s, 1H), 9.24 (dd, J=2.3, 0.9 Hz, 1H), 8.59 (dd, J=4.9, 1.6 Hz, 1H), 8.43 (dt, J=8.2, 2.0 Hz, 1H), 7.72-7.64 (m, 2H), 7.60-7.48 (m, 4H), 7.40-7.33 (m, 2H), 4.23 (s, 2H); HRMS: m/z (M+H)⁺=430.0834 (Calculated for C₂₁H₁₅F₃N₃O₂S=430.0832).

COMPOUND 59 was prepared according to the method described in Scheme 1 substituting [1,1′-biphenyl]-3-carboxylic acid for 5-(3-nitrophenyl)furan-2-carboxylic acid.

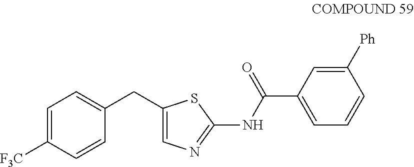 [SMILES: O=C(Nc1ncc(Cc2ccc(C(F)(F)F)cc2)s1)c1cccc(-c2ccccc2)c1]

¹H NMR (400 MHz, DMSO-d₆) δ 12.68 (s, 1H), 8.39 (td, J=1.8, 0.5 Hz, 1H), 7.95 (dddd, J=29.5, 7.8, 1.9, 1.1 Hz, 2H), 7.83-7.76 (m, 2H), 7.73-7.56 (m, 3H), 7.56-7.45 (m, 4H), 7.44-7.35 (m, 2H), 3.30 (s, 2H); HRMS: m/z (M+H)⁺=439.1074 (Calculated for C₂₄H₁₈F₃N₂OS=439.1086).

COMPOUND 74 was prepared according to the method described in Scheme 1 substituting 3-phenylpropanoic acid for 5-(3-nitrophenyl)furan-2-carboxylic acid.

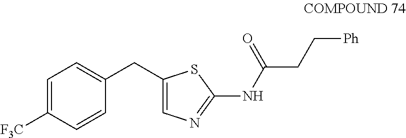 [SMILES: O=C(CCc1ccccc1)Nc1ncc(Cc2ccc(C(F)(F)F)cc2)s1]

¹H NMR (400 MHz, DMSO-d₆) δ 11.97 (s, 1H), 7.66 (dt, J=7.2, 0.9 Hz, 2H), 7.51-7.43 (m, 2H), 7.29-7.10 (m, 6H), 4.17 (s, 2H), 2.86 (t, J=7.6 Hz, 2H), 2.68 (dd, J=8.2, 6.8 Hz, 2H); HRMS: m/z (M+H)⁺=391.1091 (Calculated for C₂₀H₁₈F₃N₂OS=391.1086).

COMPOUND 67 was prepared according to the method described in Scheme 1 substituting 5-methylthiazol-2-amine for 5-(4-(trifluoromethyl)benzyl)thiazol-2-amine.

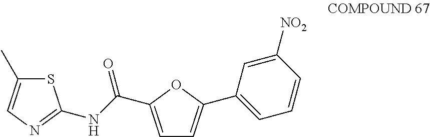 [SMILES: Cc1cnc(NC(=O)c2ccc(-c3cccc([N+](=O)[O-])c3)o2)s1]

¹H NMR (400 MHz, DMSO-d₆) δ 12.73 (s, 1H), 8.81 (s, 1H), 8.44 (d, J=7.7 Hz, 1H), 8.22 (ddd, J=8.2, 2.3, 1.0 Hz, 1H), 7.78 (t, J=8.0 Hz, 1H), 7.59 (s, 1H), 7.48 (d, J=3.7 Hz, 1H), 7.24 (q, J=1.1 Hz, 1H), 2.37 (d, J=1.3 Hz, 3H); HRMS: m/z (M+H)⁺=330.0537 (Calculated for C₁₅H₁₂N₃O₄S=330.0543).

COMPOUND 58 was prepared according to the method described in Scheme 1 substituting 4-phenylpicolinic acid for 5-(3-nitrophenyl)furan-2-carboxylic acid.

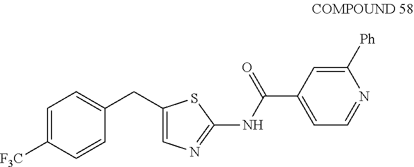 [SMILES: O=C(Nc1ncc(Cc2ccc(C(F)(F)F)cc2)s1)c1ccnc(-c2ccccc2)c1]

¹H NMR (400 MHz, DMSO-d₆) δ 12.18 (s, 1H), 8.40-8.32 (m, 2H), 8.24 (dd, J=7.9, 1.1 Hz, 1H), 8.20-8.03 (m, 2H), 7.73-7.65 (m, 2H), 7.58-7.37 (m, 6H), 4.26 (s, 2H); HRMS: m/z (M+H)⁺=440.1039 (Calculated for C₂₃H₁₇F₃N₃OS=440.1039).

COMPOUND 56 was prepared according to the method described in Scheme 1 substituting 3-benzylaniline for 5-(4-(trifluoromethyl)benzyl)thiazol-2-amine.

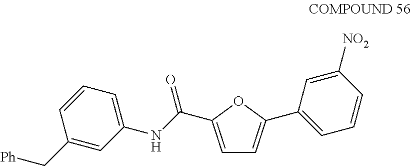 [SMILES: O=C(Nc1cccc(Cc2ccccc2)c1)c1ccc(-c2cccc([N+](=O)[O-])c2)o1]

¹H NMR (400 MHz, DMSO-d₆) δ 10.27 (s, 1H), 8.74 (t, J=2.0 Hz, 1H), 8.39 (ddd, J=7.8, 1.7, 1.0 Hz, 1H), 8.21 (ddd, J=8.2, 2.3, 1.0 Hz, 1H), 7.78 (t, J=8.0 Hz, 1H), 7.66-7.50 (m, 2H), 7.48-7.38 (m, 2H), 7.34-7.13 (m, 6H), 7.01 (dt, J=7.7, 1.2 Hz, 1H), 3.94 (s, 2H); HRMS: m/z (M+H)⁺=399.1331 (Calculated for C₂₄H₁₉N₂O₄=399.1339).

COMPOUND 61 was prepared according to the method described in Scheme 1 substituting 6-pheylpicolinic acid for 5-(3-nitrophenyl)furan-2-carboxylic acid.

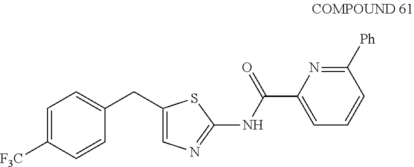 [SMILES: O=C(Nc1ncc(Cc2ccc(C(F)(F)F)cc2)s1)c1cccc(-c2ccccc2)n1]

Not enough sample to run NMR; HRMS: m/z (M+H)⁺=440.1039 (Calculated for C₂₃H₁₇F₃N₃OS=440.1039).

COMPOUND 111 was prepared according to the method described in Scheme 1 substituting 5-(trifluoromethyl)benzo[d]thiazol-2-amine for 5-(4-(trifluoromethyl)benzyl)thiazol-2-amine.

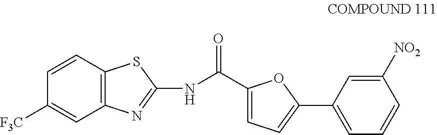 [SMILES: O=C(Nc1nc2cc(C(F)(F)F)ccc2s1)c1ccc(-c2cccc([N+](=O)[O-])c2)o1]

¹H NMR (400 MHz, DMSO-d₆) δ 13.42 (s, 1H), 8.87 (s, 1H), 8.49 (d, J=7.8 Hz, 1H), 8.33-8.21 (m, 2H), 8.09 (s, 1H), 7.85-7.72 (m, 2H), 7.70-7.62 (m, 1H), 7.55 (d, J=3.8 Hz, 1H); HRMS: m/z (M+H)⁺=434.0412 (Calculated for C₁₉H₁₁F₃N₃O₄S=434.0417).

COMPOUND 117 was prepared according to the method described in Scheme 1 substituting 1-(3-nitrophenyl)-1H-pyrazole-3-carboxylic acid for 5-(3-nitrophenyl)furan-2-carboxylic acid.

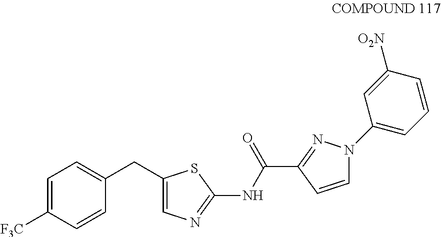 [SMILES: O=C(Nc1ncc(Cc2ccc(C(F)(F)F)cc2)s1)c1ccn(-c2cccc([N+](=O)[O-])c2)n1]

¹H NMR (400 MHz, DMSO-d₆) δ 12.51 (s, 1H), 8.95-8.83 (m, 2H), 8.50 (ddd, J=8.2, 2.2, 0.9 Hz, 1H), 8.22 (ddd, J=8.3, 2.2, 0.9 Hz, 1H), 7.84 (t, J=8.2 Hz, 1H), 7.69 (d, J=8.0 Hz, 2H), 7.52 (d, J=8.0 Hz, 2H), 7.38 (d, J=1.0 Hz, 1H), 7.18 (d, J=2.6 Hz, 1H), 4.24 (s, 2H); HRMS: m/z (M+H)⁺=474.0833 (Calculated for C₂₁H₁₅F₃N₅O₃S=474.0842).

COMPOUND 118 was prepared according to the method described in Scheme 1 substituting 2-phenyl-1H-imidazole-4-carboxylic acid for 5-(3-nitrophenyl)furan-2-carboxylic acid.

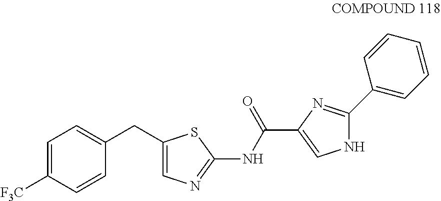 [SMILES: O=C(Nc1ncc(Cc2ccc(C(F)(F)F)cc2)s1)c1c[nH]c(-c2ccccc2)n1]

¹H NMR (400 MHz, DMSO-d6) δ 8.13 (s, 1H), 8.08-8.01 (m, 2H), 7.72-7.64 (m, 2H), 7.55-7.36 (m, 5H), 7.36-7.30 (m, 1H), 4.22 (s, 2H); HRMS: m/z (M+H)=429.1011 (Calculated for C₂₁H₁₆F₃N₄OS=429.0991).

COMPOUND 164 was prepared according to the method described in Scheme 1 substituting 1-phenyl-1H-imidazole-4-carboxylic acid for 5-(3-nitrophenyl)furan-2-carboxylic acid.

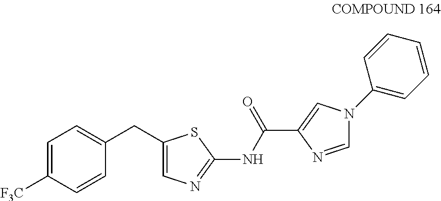 [SMILES: O=C(Nc1ncc(Cc2ccc(C(F)(F)F)cc2)s1)c1cn(-c2ccccc2)cn1]

¹H NMR (400 MHz, DMSO-d6) δ 12.51 (s, 1H), 8.94-8.84 (m, 2H), 8.50 (d, J=8.6 Hz, 1H), 8.26-8.18 (m, 1H), 7.84 (t, J=8.2 Hz, 1H), 7.69 (d, J=8.1 Hz, 2H), 7.52 (d, J=8.0 Hz, 2H), 7.38 (s, 1H), 7.18 (s, 1H), 4.24 (s, 2H), NMR run with very little sample; HRMS: m/z (M+H)⁺=429.0990 (Calculated for C₂₁H₁₆F₃N₄OS=429.0991).

COMPOUND 137 was prepared according to the method described in Scheme 1 substituting N-phenylbenzene-1,3-diamine for 5-(4-(trifluoromethyl)benzyl)thiazol-2-amine.

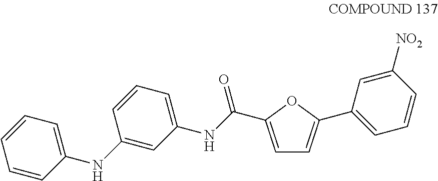 [SMILES: O=C(Nc1cccc(Nc2ccccc2)c1)c1ccc(-c2cccc([N+](=O)[O-])c2)o1]

¹H NMR (400 MHz, DMSO-d₆) δ 10.20 (s, 1H), 8.74 (t, J=2.0 Hz, 1H), 8.39 (ddd, J=7.8, 1.7, 1.0 Hz, 1H), 8.25-8.17 (m, 2H), 7.78 (t, J=8.0 Hz, 1H), 7.61 (q, J=1.5 Hz, 1H), 7.49-7.40 (m, 2H), 7.29-7.06 (m, 6H), 6.87-6.76 (m, 2H); HRMS: m/z (M+H)⁺=400.1289 (Calculated for C₂₃H₁₈N₃O₄=400.1292).

COMPOUND 142 was prepared according to the method described in Scheme 1 substituting 6-benzylpyridin-2-amine for 5-(4-(trifluoromethyl)benzyl)thiazol-2-amine.

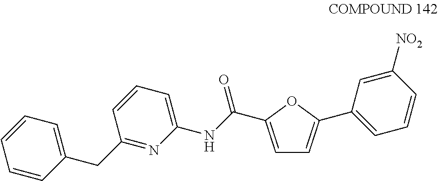 [SMILES: O=C(Nc1cccc(Cc2ccccc2)n1)c1ccc(-c2cccc([N+](=O)[O-])c2)o1]

¹H NMR (400 MHz, DMSO-d₆) δ 10.97 (s, 1H), 8.79 (ddd, J=2.2, 1.7, 0.4 Hz, 1H), 8.44 (ddd, J=7.8, 1.7, 1.0 Hz, 1H), 8.28-8.17 (m, 1H), 8.00 (dd, J=8.3, 0.8 Hz, 1H), 7.82-7.71 (m, 2H), 7.59 (d, J=3.7 Hz, 1H), 7.50-7.40 (m, 1H), 7.39-7.15 (m, 5H), 7.00 (dd, J=7.5, 0.9 Hz, 1H), 4.09 (s, 2H); HRMS: m/z (M+H)⁺=400.1290 (Calculated for C₂₃H₁₈N₃O₄=400.1292).

COMPOUND 150 was prepared according to the method described in Scheme 1 substituting 4-benzylpyridin-2-amine for 5-(4-(trifluoromethyl)benzyl)thiazol-2-amine.

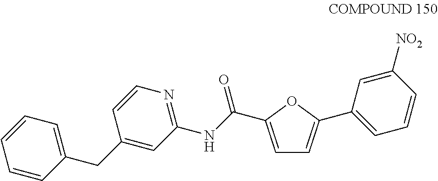 [SMILES: O=C(Nc1cc(Cc2ccccc2)ccn1)c1ccc(-c2cccc([N+](=O)[O-])c2)o1]

¹H NMR (400 MHz, DMSO-d₆) δ 11.01 (s, 1H), 8.82-8.77 (m, 1H), 8.43 (ddd, J=7.9, 1.7, 1.0 Hz, 1H), 8.29 (dd, J=5.0, 0.8 Hz, 1H), 8.21 (ddd, J=8.2, 2.3, 1.0 Hz, 1H), 8.05 (dq, J=1.3, 0.6 Hz, 1H), 7.77 (t, J=8.0 Hz, 1H), 7.56 (d, J=3.7 Hz, 1H), 7.44 (d, J=3.7 Hz, 1H), 7.37-7.17 (m, 5H), 7.09-7.02 (m, 1H), 4.00 (s, 2H); HRMS: m/z (M+H)⁺=400.1291 (Calculated for C₂₃H₁₈N₃O₄=400.1292).

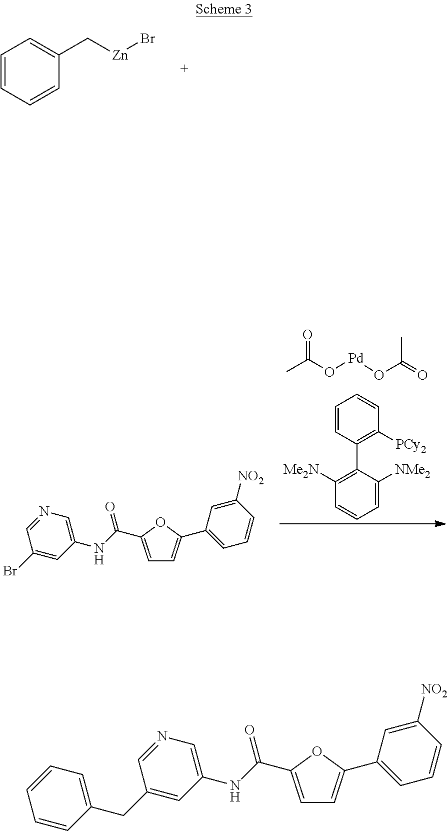

Scheme 3 To a solution of N-(5-bromopyridin-3-yl)-5-(3-nitrophenyl)furan-2-carboxamide (50 mg, 0.13 mmol), 2′-(dicyclohexylphosphino)-N2,N2,N6,N6-tetramethyl-[1,1′-biphenyl]-2,6-diamine (6 mg, 0.013 mmol), and palladium(II) acetate (2 mg, 5 mol %) in THF (0.5 mL) was added benzylzinc(II) bromide (0.5 mL of 0.5M solution in THF). The reaction mixture was heated with stirring at 60° C. for 1 hr. The reaction mixture was filtered through an Agilent PL-Thiol MP SPE cartridge to remove palladium. The organic layer was concentrated under a stream of air. The residue was taken up in DMSO and subsequently purified by reverse phase chromatography to give COMPOUND 170.

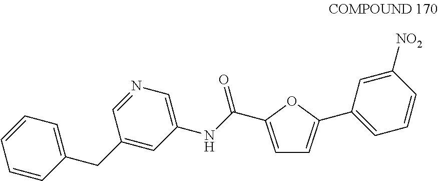 [SMILES: O=C(Nc1cncc(Cc2ccccc2)c1)c1ccc(-c2cccc([N+](=O)[O-])c2)o1]

¹H NMR (400 MHz, DMSO-d₆) δ 10.59 (s, 1H), 8.91 (d, J=2.3 Hz, 1H), 8.73 (dd, J=2.2, 1.7 Hz, 1H), 8.38 (ddd, J=7.8, 1.7, 1.0 Hz, 2H), 8.22 (ddd, J=8.2, 2.3, 1.0 Hz, 1H), 8.07 (t, J=2.1 Hz, 1H), 7.84-7.75 (m, 1H), 7.51-7.44 (m, 2H), 7.37-7.17 (m, 5H), 4.04 (s, 2H); HRMS: m/z (M+H)⁺=400.1280 (Calculated for C₂₃H₁₈N₃O₄=400.1292).

COMPOUND 177 was prepared according to the method described in Scheme 3 substituting N-(2-chloropyridin-4-yl)-5-(3-nitrophenyl)furan-2-carboxamide for N-(5-bromopyridin-3-yl)-5-(3-nitrophenyl)furan-2-carboxamide.

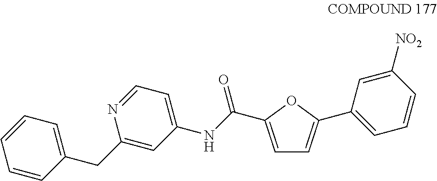 [SMILES: O=C(Nc1ccnc(Cc2ccccc2)c1)c1ccc(-c2cccc([N+](=O)[O-])c2)o1]

¹H NMR (400 MHz, DMSO-d₆) δ 11.13 (s, 1H), 8.73 (ddd, J=2.2, 1.7, 0.4 Hz, 1H), 8.61 (d, J=6.4 Hz, 1H), 8.38 (ddd, J=7.8, 1.7, 1.0 Hz, 1H), 8.25 (ddd, J=8.2, 2.3, 1.0 Hz, 1H), 8.04 (s, 1H), 7.90 (s, 1H), 7.81 (ddd, J=8.2, 7.8, 0.4 Hz, 1H), 7.61 (d, J=3.8 Hz, 1H), 7.52 (d, J=3.7 Hz, 1H), 7.41-7.24 (m, 5H), 4.26 (s, 2H); HRMS: m/z (M+H)⁺=400.1302 (Calculated for C₂₃H₁₈N₃O₄=400.1292).

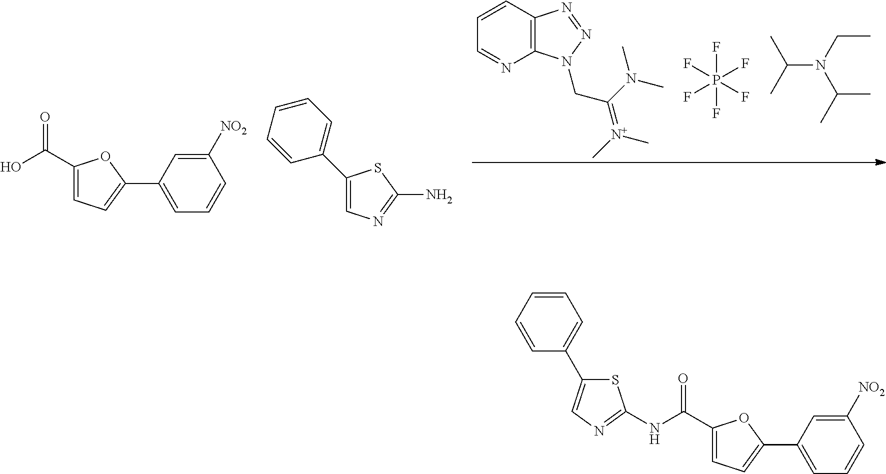

Scheme 4 COMPOUND 82 To a solution of 5-(3-nitrophenyl)furan-2-carboxylic acid (50 mg, 0.214 mmol) in DMF (1 ml) were added HATU (90 mg, 0.236 mmol) and DIPEA (0.112 ml, 0.643 mmol). The mixture was stirred at rt for 10 min and then a solution of 5-phenylthiazol-2-amine (41.6 mg, 0.236 mmol) in DMF (1 ml) was added to the reaction mixture. The reaction mixture was stirred at rt for overnight. The crude product was purified by reverse phase chromatography to give COMPOUND 82.

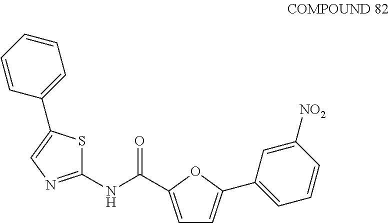 [SMILES: O=C(Nc1ncc(-c2ccccc2)s1)c1ccc(-c2cccc([N+](=O)[O-])c2)o1]

¹H NMR (400 MHz, DMSO-d6) δ 8.85 (s, 1H), 8.48 (d, J=7.9 Hz, 1H), 8.25 (ddd, J=8.2, 2.3, 1.0 Hz, 1H), 8.00 (s, 1H), 7.86-7.78 (m, 1H), 7.74-7.64 (m, 4H), 7.53 (d, J=3.7 Hz, 1H), 7.49-7.41 (m, 2H), 7.37-7.30 (m, 1H); Method 1, retention time: 6.581 min; HRMS: m/z (M+H)⁺=392.0684 (Calculated for C₂₀H₁₄N₃O₄S=392.0700).

COMPOUND 151 was prepared according to the method described in Scheme 4 substituting 5-phenyl-1H-imidazole-2-carboxylic acid TFA salt for 5-(3-nitrophenyl)furan-2-carboxylic acid.

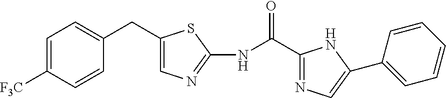 [SMILES: O=C(Nc1ncc(Cc2ccc(C(F)(F)F)cc2)s1)c1ncc(-c2ccccc2)[nH]1]

¹H NMR (400 MHz, DMSO-d₆) δ 11.87 (s, 1H), 7.91 (d, J=7.7 Hz, 3H), 7.72-7.65 (m, 2H), 7.55-7.48 (m, 2H), 7.43-7.34 (m, 3H), 7.26 (t, J=7.3 Hz, 1H), 4.24 (s, 2H); HRMS: m/z (M+H)⁺=429.0993 (Calculated for C₂₁H₁₆F₃N₄OS=429.0991).

COMPOUND 85 was prepared according to the method described in Scheme 4 substituting 4-benzylthiazol-2-amine for 5-phenylthiazol-2-amine.

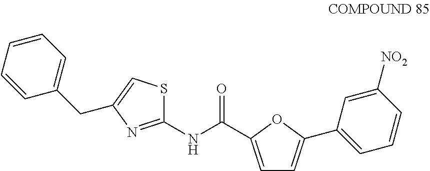 [SMILES: O=C(Nc1nc(Cc2ccccc2)cs1)c1ccc(-c2cccc([N+](=O)[O-])c2)o1]

¹H NMR (400 MHz, DMSO-d6) δ 12.86 (s, 1H), 8.90-8.78 (m, 1H), 8.45 (dt, J=7.9, 1.3 Hz, 1H), 8.23 (ddd, J=8.2, 2.3, 1.0 Hz, 1H), 7.87-7.70 (m, 1H), 7.62 (d, J=3.8 Hz, 1H), 7.49 (d, J=3.7 Hz, 1H), 7.34-7.25 (m, 4H), 7.24-7.17 (m, 1H), 6.93 (s, 1H), 4.01 (s, 2H); Method 1, retention time: 6.648 min; HRMS: m/z (M+H)⁺=406.0874 (Calculated for C₂₁H₁₆N₃O₄S=406.0856).

COMPOUND 86 was prepared according to the method described in Scheme 4 substituting 5-(4-(trifluoromethyl)benzyl)thiazol-2-amine for 5-phenylthiazol-2-amine and substituting 2-phenyloxazole-5-carboxylic acid for 5-(3-nitrophenyl)furan-2-carboxylic acid.

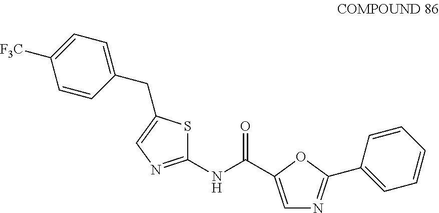 [SMILES: O=C(Nc1ncc(Cc2ccc(C(F)(F)F)cc2)s1)c1cnc(-c2ccccc2)o1]

¹H NMR (400 MHz, DMSO-d6) δ 12.94 (s, 1H), 8.22 (s, 1H), 8.20-8.14 (m, 2H), 7.71 (dq, J=1.5, 0.8 Hz, 1H), 7.69 (dt, J=1.5, 0.8 Hz, 1H), 7.63-7.57 (m, 3H), 7.54 (dd, J=1.3, 0.7 Hz, 1H), 7.53 (dt, J=1.4, 0.7 Hz, 1H), 7.42-7.39 (m, 1H), 4.25 (s, 2H); Method 1, retention time: 6.567 min; HRMS: m/z (M+H)⁺=430.0820 (Calculated for C₂₁H₁₅F₃N₃O₂S=430.0832).

COMPOUND 83 was prepared according to the method described in Scheme 4 substituting 5-(4-(trifluoromethyl)benzyl)thiazol-2-amine for 5-phenylthiazol-2-amine and substituting 5-phenylthiophene-2-carboxylic acid for 5-(3-nitrophenyl)furan-2-carboxylic acid.

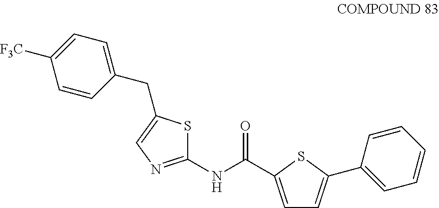 [SMILES: O=C(Nc1ncc(Cc2ccc(C(F)(F)F)cc2)s1)c1ccc(-c2ccccc2)s1]

¹H NMR (400 MHz, DMSO-d6) δ 12.65 (s, 1H), 8.21 (s, 1H), 7.78-7.72 (m, 2H), 7.71 (dq, J=1.4, 0.8 Hz, 1H), 7.69 (dd, J=1.4, 0.7 Hz, 1H), 7.63 (d, J=4.0 Hz, 1H), 7.54 (dd, J=1.4, 0.7 Hz, 1H), 7.52 (dp, J=2.0, 1.0 Hz, 1H), 7.50-7.44 (m, 2H), 7.43-7.35 (m, 2H), 4.23 (s, 2H); Method 1, retention time: 7.105 min; HRMS: m/z (M+Na)⁺=467.0462 (Calculated for C₂₂H₁₅F₃N₂NaOS₂=467.0470).

COMPOUND 108 was prepared according to the method described in Scheme 4 substituting 5-(4-(trifluoromethyl)benzyl)thiazol-2-amine for 5-phenylthiazol-2-amine and substituting 4-phenylbutanoic acid for 5-(3-nitrophenyl)furan-2-carboxylic acid.

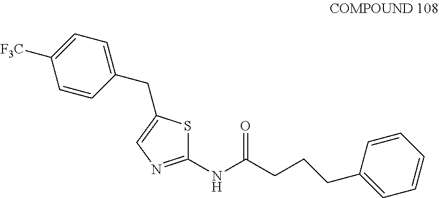 [SMILES: O=C(CCCc1ccccc1)Nc1ncc(Cc2ccc(C(F)(F)F)cc2)s1]

¹H NMR (400 MHz, DMSO-d6) δ 11.95 (s, 1H), 7.72-7.64 (m, 2H), 7.49 (ddt, J=7.6, 1.5, 0.7 Hz, 2H), 7.32-7.22 (m, 3H), 7.22-7.12 (m, 3H), 4.19 (s, 2H), 2.57 (dd, J=8.6, 6.8 Hz, 2H), 2.40 (t, J=7.4 Hz, 2H), 1.92-1.80 (m, 2H); Method 1, retention time: 6.691 min; HRMS: m/z (M+H)⁺=405.1251 (Calculated for C₂₁H₂₀F₃N₂OS=405.1243).

COMPOUND 98 was prepared according to the method described in Scheme 4 substituting 5-(4-(trifluoromethyl)benzyl)thiazol-2-amine for 5-phenylthiazol-2-amine and substituting 5-bromofuran-2-carboxylic acid for 5-(3-nitrophenyl)furan-2-carboxylic acid.

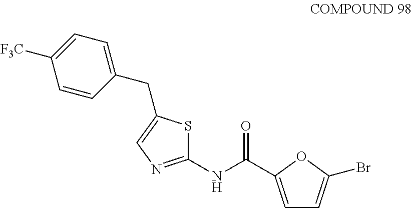 [SMILES: O=C(Nc1ncc(Cc2ccc(C(F)(F)F)cc2)s1)c1ccc(Br)o1]

¹H NMR (400 MHz, DMSO-d6) δ 12.56 (s, 1H), 7.83-7.63 (m, 2H), 7.61 (s, 1H), 7.58-7.47 (m, 2H), 7.36 (s, 1H), 6.85 (d, J=3.6 Hz, 1H), 4.22 (s, 2H); Method 1, retention time: 6.397 min; HRMS: m/z (M+H)⁺=432.9670 (Calculated for C₁₆H₁₁BrF₃N₂O₂S=432.9651).

COMPOUND 99 was prepared according to the method described in Scheme 4 substituting 5-(4-(trifluoromethyl)benzyl)thiazol-2-amine for 5-phenylthiazol-2-amine and substituting 2-phenylthiazole-5-carboxylic acid for 5-(3-nitrophenyl)furan-2-carboxylic acid.

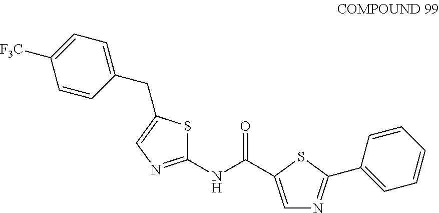 [SMILES: O=C(Nc1ncc(Cc2ccc(C(F)(F)F)cc2)s1)c1cnc(-c2ccccc2)s1]

¹H NMR (400 MHz, DMSO-d6) δ 12.91 (s, 1H), 8.80 (s, 1H), 8.07-7.95 (m, 2H), 7.74-7.63 (m, 2H), 7.59-7.46 (m, 5H), 7.38 (s, 1H), 4.22 (s, 2H); Method 1, retention time: 6.826 min; HRMS: m/z (M+H)⁺=446.0615 (Calculated for C₂₁H₁₅F₃N₃OS₂=446.0603).

COMPOUND 103 was prepared according to the method described in Scheme 4 substituting 1-benzylpiperidin-3-amine for 5-phenylthiazol-2-amine.

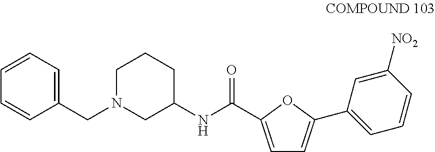 [SMILES: O=C(NC1CCCN(Cc2ccccc2)C1)c1ccc(-c2cccc([N+](=O)[O-])c2)o1]

¹H NMR (400 MHz, DMSO-d6) δ 8.67 (dd, J=4.8, 2.8 Hz, 2H), 8.34 (ddd, J=7.8, 1.7, 1.0 Hz, 1H), 8.22 (ddd, J=8.3, 2.3, 1.0 Hz, 1H), 7.79 (t, J=8.0 Hz, 1H), 7.58-7.45 (m, 5H), 7.41 (d, J=3.6 Hz, 1H), 7.28 (d, J=3.6 Hz, 1H), 4.45-4.34 (m, 2H), 4.30-4.17 (m, 1H), 3.52-3.37 (m, 2H), 2.95-2.71 (m, 2H), 2.06-1.87 (m, 2H), 1.85-1.52 (m, 2H); Method 1, retention time: 4.288 min; HRMS: m/z (M+H)=406.1752 (Calculated for C₂₃H₂₄N₃O₄=406.1761).

COMPOUND 189 was prepared according to the method described in Scheme 4 substituting 5-(4-(trifluoromethyl)benzyl)thiazol-2-amine for 5-phenylthiazol-2-amine and substituting 1-(3-methylbenzyl)-1H-pyrazole-3-carboxylic acid for 5-(3-nitrophenyl)furan-2-carboxylic acid.

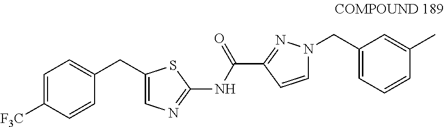 [SMILES: Cc1cccc(Cn2ccc(C(=O)Nc3ncc(Cc4ccc(C(F)(F)F)cc4)s3)n2)c1]

¹H NMR (400 MHz, DMSO-d6) δ 11.94 (s, 1H), 7.98 (d, J=2.4 Hz, 1H), 7.70 (dq, J=1.4, 0.8 Hz, 1H), 7.68 (dt, J=1.5, 0.7 Hz, 1H), 7.52 (tt, J=1.4, 0.7 Hz, 1H), 7.51 (dq, J=1.5, 0.7 Hz, 1H), 7.35-7.32 (m, 1H), 7.28-7.20 (m, 1H), 7.14-7.07 (m, 3H), 6.99 (d, J=2.4 Hz, 1H), 5.39 (s, 2H), 4.22 (s, 2H), 2.28 (q, J=0.6 Hz, 3H); Method 1, retention time: 6.737 min; HRMS: m/z (M+H)⁺=457.1312 (Calculated for C₂₃H₂₀F₃N₄OS=457.1304).

COMPOUND 190 was prepared according to the method described in Scheme 4 substituting 5-(4-(trifluoromethyl)benzyl)thiazol-2-amine for 5-phenylthiazol-2-amine and substituting 1-((2-methylpyrimidin-4-yl)methyl))-1H-pyrazole-3-carboxylic acid for 5-(3-nitrophenyl)furan-2-carboxylic acid.

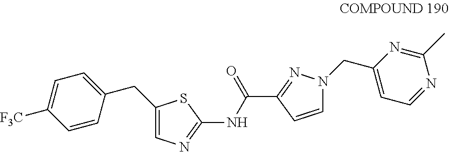 [SMILES: Cc1nccc(Cn2ccc(C(=O)Nc3ncc(Cc4ccc(C(F)(F)F)cc4)s3)n2)n1]

¹H NMR (400 MHz, DMSO-d6) δ 12.02 (s, 1H), 8.65 (d, J=5.2 Hz, 1H), 8.06 (d, J=2.4 Hz, 1H), 7.70 (dq, J=1.4, 0.8 Hz, 1H), 7.69-7.66 (m, 1H), 7.52 (dd, J=1.4, 0.7 Hz, 1H), 7.51 (dd, J=1.5, 0.8 Hz, 1H), 7.37-7.31 (m, 1H), 7.06 (d, J=2.4 Hz, 1H), 6.92 (dq, J=5.2, 0.6 Hz, 1H), 5.55 (s, 2H), 4.23 (s, 2H), 2.60 (d, J=0.5 Hz, 3H); Method 1, retention time: 6.737 min; HRMS: m/z (M+H)⁺=459.1224 (Calculated for C₂₁H₁₈F₃N₆OS=459.1209).

COMPOUND 191 was prepared according to the method described in Scheme 4 substituting 5-(4-(trifluoromethyl)benzyl)thiazol-2-amine for 5-phenylthiazol-2-amine and substituting 1-((2-methylpyridin-4-yl)methyl))-1H-pyrazole-3-carboxylic acid for 5-(3-nitrophenyl)furan-2-carboxylic acid.

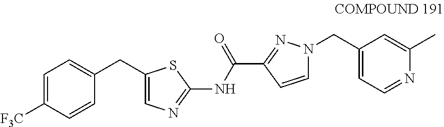 [SMILES: Cc1cc(Cn2ccc(C(=O)Nc3ncc(Cc4ccc(C(F)(F)F)cc4)s3)n2)ccn1]

¹H NMR (400 MHz, DMSO-d6) δ 12.03 (s, 1H), 8.65-8.59 (m, 1H), 8.07 (d, J=2.4 Hz, 1H), 7.70 (dq, J=1.3, 0.8 Hz, 1H), 7.68 (dd, J=1.4, 0.7 Hz, 1H), 7.52 (dd, J=1.3, 0.7 Hz, 1H), 7.51-7.48 (m, 1H), 7.44 (s, 1H), 7.40 (d, J=6.0 Hz, 1H), 7.37-7.33 (m, 1H), 7.08 (d, J=2.4 Hz, 1H), 5.65 (s, 2H), 4.23 (s, 2H), 2.59 (s, 3H); Method 1, retention time: 4.687 min; HRMS: m/z (M+H)⁺=458.1266 (Calculated for C₂₂H₁₉F₃N₅OS=458.1257).

COMPOUND 231 was prepared according to the method described in Scheme 4 substituting 5-(4-(trifluoromethyl)benzyl)thiazol-2-amine for 5-phenylthiazol-2-amine and substituting 1-(2-morpholinoethyl)-1H-pyrazole-3-carboxylic acid for 5-(3-nitrophenyl)furan-2-carboxylic acid.

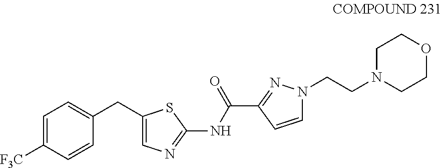 [SMILES: O=C(Nc1ncc(Cc2ccc(C(F)(F)F)cc2)s1)c1ccn(CCN2CCOCC2)n1]

Method 1, retention time: 4.481 min; HRMS: m/z (M+H)⁺=466.1515 (Calculated for C₂₁H₂₃F₃N₅O₂S=466.1519).

COMPOUND 194 was prepared according to the method described in Scheme 4 substituting 5-(4-(trifluoromethyl)benzyl)thiazol-2-amine for 5-phenylthiazol-2-amine and substituting 5-methyl-1-(2-morpholinoethyl)-1H-pyrazole-3-carboxylic acid for 5-(3-nitrophenyl)furan-2-carboxylic acid.

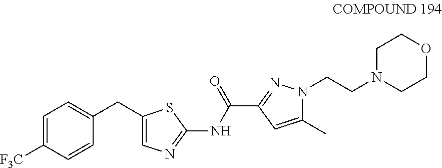 [SMILES: Cc1cc(C(=O)Nc2ncc(Cc3ccc(C(F)(F)F)cc3)s2)nn1CCN1CCOCC1]

¹H NMR (400 MHz, DMSO-d6) δ 12.61 (s, 1H), 9.73 (s, 1H), 7.68 (d, J=7.9 Hz, 2H), 7.50 (d, J=7.9 Hz, 2H), 7.40 (s, 1H), 4.79 (s, 2H), 4.22 (s, 2H), 3.36 (m, 8H), 2.91 (s, 2H), 2.20 (s, 3H); Method 1, retention time: 4.616 min; HRMS: m/z (M+H)⁺=480.1693 (Calculated for C₂₂H₂₅F₃N₅O₂S=480.1676).

COMPOUND 192 was prepared according to the method described in Scheme 4 substituting 5-(4-(trifluoromethyl)benzyl)thiazol-2-amine for 5-phenylthiazol-2-amine and substituting 1-(pyridin-2-ylmethyl)-1H-pyrazole-3-carboxylic acid for 5-(3-nitrophenyl)furan-2-carboxylic acid.

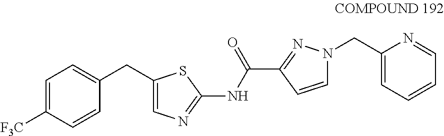 [SMILES: O=C(Nc1ncc(Cc2ccc(C(F)(F)F)cc2)s1)c1ccn(Cc2ccccn2)n1]

¹H NMR (400 MHz, DMSO-d6) δ 11.97 (s, 1H), 8.57-8.50 (m, 1H), 8.03 (d, J=2.4 Hz, 1H), 7.79 (td, J=7.7, 1.8 Hz, 1H), 7.69 (d, J=8.0 Hz, 2H), 7.51 (d, J=8.0 Hz, 2H), 7.33 (d, J=7.6 Hz, 2H), 7.19 (d, J=7.9 Hz, 1H), 7.03 (d, J=2.3 Hz, 1H), 5.55 (s, 2H), 4.22 (s, 2H); Method 1, retention time: 5.337 min; HRMS: m/z (M+H)⁺=444.1112 (Calculated for C₂₁H₁₇F₃N₅OS=444.1100).

COMPOUND 193 was prepared according to the method described in Scheme 4 substituting 5-(4-(trifluoromethyl)benzyl)thiazol-2-amine for 5-phenylthiazol-2-amine and substituting 5-methyl-1-(pyridin-2-ylmethyl)-1H-pyrazole-3-carboxylic acid for 5-(3-nitrophenyl)furan-2-carboxylic acid.

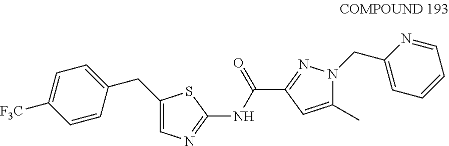 [SMILES: Cc1cc(C(=O)Nc2ncc(Cc3ccc(C(F)(F)F)cc3)s2)nn1Cc1ccccn1]

¹H NMR (400 MHz, DMSO-d6) δ 11.82 (s, 1H), 8.58-8.45 (m, 1H), 7.79 (td, J=7.8, 1.7 Hz, 1H), 7.69 (d, J=8.0 Hz, 2H), 7.51 (d, J=8.0 Hz, 2H), 7.32 (d, J=10.0 Hz, 2H), 7.13 (d, J=7.9 Hz, 1H), 6.79 (s, 1H), 5.50 (s, 2H), 4.22 (s, 2H), 2.30 (s, 3H); Method 1, retention time: 5.557 min; HRMS: m/z (M+H)⁺=458.1260 (Calculated for C₂₂H₁₉F₃N₅OS=458.1257).

COMPOUND 213 was prepared according to the method described in Scheme 4 substituting 5-((5-methylfuran-2-yl)methyl)thiazol-2-amine for 5-phenylthiazol-2-amine and substituting 1-phenyl-1H-pyrazole-3-carboxylic acid for 5-(3-nitrophenyl)furan-2-carboxylic acid.

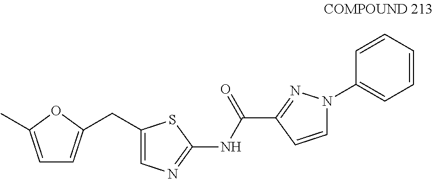 [SMILES: Cc1ccc(Cc2cnc(NC(=O)c3ccn(-c4ccccc4)n3)s2)o1]

¹H NMR (400 MHz, DMSO-d6) δ 12.27 (s, 1H), 8.68 (d, J=2.6 Hz, 1H), 8.05 (d, J=7.8 Hz, 2H), 7.56 (t, J=7.9 Hz, 2H), 7.40 (t, J=7.4 Hz, 1H), 7.34 (s, 1H), 7.18 (d, J=2.6 Hz, 1H), 6.07 (d, J=3.0 Hz, 1H), 6.03-5.92 (m, 1H), 4.12 (s, 2H), 2.23 (s, 3H); Method 1, retention time: 6.108 min; HRMS: m/z (M+H)⁺=365.1070 (Calculated for C₁₉H₁₇N₄O₂S=365.1067).

COMPOUND 214 was prepared according to the method described in Scheme 4 substituting 5-(2-methylbenzyl)thiazol-2-amine for 5-phenylthiazol-2-amine and substituting 1-phenyl-1H-pyrazole-3-carboxylic acid for 5-(3-nitrophenyl)furan-2-carboxylic acid.

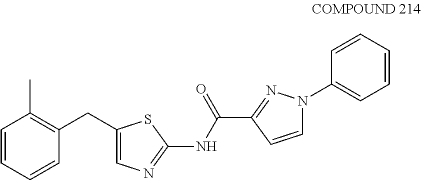 [SMILES: Cc1ccccc1Cc1cnc(NC(=O)c2ccn(-c3ccccc3)n2)s1]

¹H NMR (400 MHz, DMSO-d6) δ 12.23 (s, 1H), 8.67 (d, J=2.6 Hz, 1H), 8.10-7.97 (m, 2H), 7.56 (t, J=7.9 Hz, 2H), 7.40 (t, J=7.4 Hz, 1H), 7.31-7.08 (m, 6H), 4.11 (s, 2H), 2.30 (s, 3H); Method 1, retention time: 6.422 min; HRMS: m/z (M+H)⁺=375.1284 (Calculated for C₂₁H₁₉N₄OS=375.1274).

COMPOUND 110 was prepared according to the method described in Scheme 4 substituting 5-(4-(trifluoromethyl)benzyl)thiazol-2-amine for 5-phenylthiazol-2-amine and substituting 1-phenyl-1H-pyrazole-3-carboxylic acid for 5-(3-nitrophenyl)furan-2-carboxylic acid.

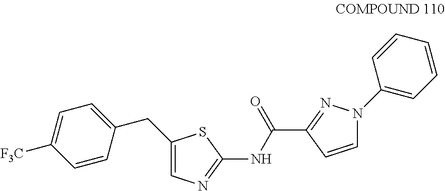 [SMILES: O=C(Nc1ncc(Cc2ccc(C(F)(F)F)cc2)s1)c1ccn(-c2ccccc2)n1]

HRMS: m/z (M+H)⁺=429.1012 (Calculated for C₂₁H₁₆F₃N₄OS=429.0991).

COMPOUND 215 was prepared according to the method described in Scheme 4 substituting 5-(2-fluorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

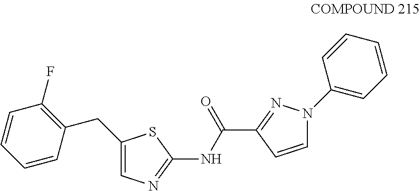 [SMILES: O=C(Nc1ncc(Cc2ccccc2F)s1)c1ccn(-c2ccccc2)n1]

¹H NMR (400 MHz, DMSO-d6) δ 12.27 (s, 1H), 8.67 (d, J=2.6 Hz, 1H), 8.06-8.01 (m, 2H), 7.59-7.52 (m, 2H), 7.40 (tq, J=7.6, 1.3 Hz, 2H), 7.36-7.28 (m, 2H), 7.24-7.14 (m, 3H), 4.16 (s, 2H); Method 1, retention time: 6.222 min; HRMS: m/z (M+H)⁺=379.1027 (Calculated for C₂₀H₁₆FN₄OS=379.1023).

COMPOUND 216 was prepared according to the method described in Scheme 4 substituting 5-(2-chlorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

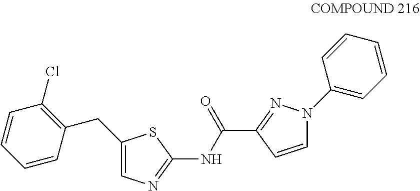 [SMILES: O=C(Nc1ncc(Cc2ccccc2Cl)s1)c1ccn(-c2ccccc2)n1]

¹H NMR (400 MHz, DMSO-d6) δ 12.27 (s, 1H), 8.67 (d, J=2.6 Hz, 1H), 8.05 (t, J=0.9 Hz, 1H), 8.03 (t, J=1.1 Hz, 1H), 7.59-7.53 (m, 2H), 7.47 (ddd, J=7.4, 5.6, 1.9 Hz, 2H), 7.43-7.37 (m, 1H), 7.37-7.28 (m, 3H), 7.16 (d, J=2.6 Hz, 1H), 4.24 (d, J=1.0 Hz, 2H); Method 1, retention time: 6.495 min; HRMS: m/z (M+H)⁺=395.0726 (Calculated for C₂₀H₁₆ClN₄OS=395.0728).

COMPOUND 217 was prepared according to the method described in Scheme 4 substituting 5-(3-methylbenzyl)thiazol-2-amine for 5-phenylthiazol-2-amine and substituting 1-phenyl-1H-pyrazole-3-carboxylic acid for 5-(3-nitrophenyl)furan-2-carboxylic acid.

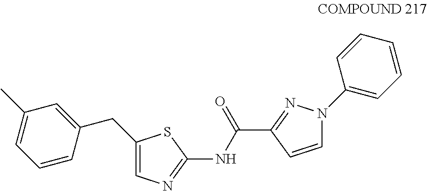 [SMILES: Cc1cccc(Cc2cnc(NC(=O)c3ccn(-c4ccccc4)n3)s2)c1]

¹H NMR (400 MHz, DMSO-d6) δ 12.23 (s, 1H), 8.67 (d, J=2.6 Hz, 1H), 8.05 (d, J=1.3 Hz, 1H), 8.03 (dd, J=2.1, 0.9 Hz, 1H), 7.59-7.52 (m, 2H), 7.43-7.37 (m, 1H), 7.34-7.31 (m, 1H), 7.22 (t, J=7.5 Hz, 1H), 7.16 (d, J=2.6 Hz, 1H), 7.13-7.02 (m, 3H), 4.08 (s, 2H), 2.29 (s, 3H); Method 1, retention time: 6.483 min; HRMS: m/z (M+H)⁺=375.1276 (Calculated for C₂₁H₁₉N₄OS=375.1274).

COMPOUND 218 was prepared according to the method described in Scheme 4 substituting 5-(3-fluorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

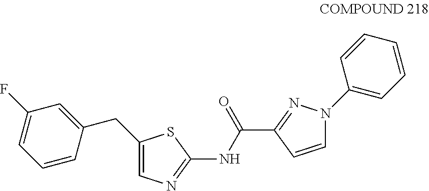 [SMILES: O=C(Nc1ncc(Cc2cccc(F)c2)s1)c1ccn(-c2ccccc2)n1]

¹H NMR (400 MHz, DMSO-d6) δ 12.25 (s, 1H), 8.65 (d, J=2.6 Hz, 1H), 8.03 (d, J=1.3 Hz, 1H), 8.01 (t, J=1.1 Hz, 1H), 7.58-7.49 (m, 2H), 7.41-7.32 (m, 3H), 7.17-7.09 (m, 3H), 7.05 (dddd, J=9.1, 8.3, 2.6, 1.1 Hz, 1H), 4.14 (s, 2H); Method 1, retention time: 6.211 min; HRMS: m/z (M+H)⁺=379.1004 (Calculated for C₂₀H₁₆FN₄OS=379.1023).

COMPOUND 219 was prepared according to the method described in Scheme 4 substituting 5-(3-methoxybenzyl)thiazol-2-amine for 5-phenylthiazol-2-amine and substituting 1-phenyl-1H-pyrazole-3-carboxylic acid for 5-(3-nitrophenyl)furan-2-carboxylic acid.

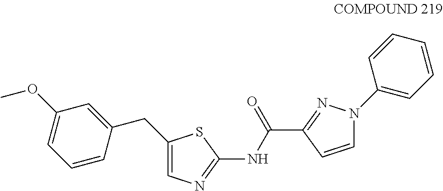 [SMILES: COc1cccc(Cc2cnc(NC(=O)c3ccn(-c4ccccc4)n3)s2)c1]

¹H NMR (400 MHz, DMSO-d6) δ 12.23 (s, 1H), 8.67 (d, J=2.6 Hz, 1H), 8.07-8.01 (m, 2H), 7.60-7.52 (m, 2H), 7.46-7.36 (m, 1H), 7.36-7.32 (m, 1H), 7.25 (t, J=8.1 Hz, 1H), 7.17 (d, J=2.6 Hz, 1H), 6.90-6.84 (m, 2H), 6.81 (ddd, J=8.2, 2.5, 1.1 Hz, 1H), 4.09 (s, 2H), 3.74 (s, 3H); Method 1, retention time: 6.112 min; HRMS: m/z (M+H)⁺=391.1226 (Calculated for C₂₁H₁₉N₄O₂S=391.1223).

COMPOUND 220 was prepared according to the method described in Scheme 4 substituting 5-(3-chlorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

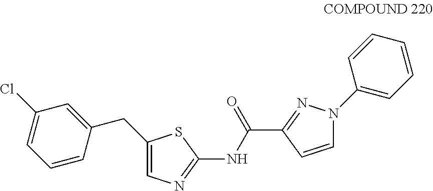 [SMILES: O=C(Nc1ncc(Cc2cccc(Cl)c2)s1)c1ccn(-c2ccccc2)n1]

¹H NMR (400 MHz, DMSO-d6) δ 12.28 (s, 1H), 8.67 (d, J=2.6 Hz, 1H), 8.05 (t, J=1.6 Hz, 1H), 8.04-8.01 (m, 1H), 7.59-7.52 (m, 2H), 7.43-7.34 (m, 4H), 7.30 (ddt, J=11.7, 7.5, 1.4 Hz, 2H), 7.17 (d, J=2.6 Hz, 1H), 4.16 (s, 2H); Method 1, retention time: 6.508 min; HRMS: m/z (M+H)⁺=395.0737 (Calculated for C₂₀H₁₆ClN₄OS=395.0728).

COMPOUND 221 was prepared according to the method described in Scheme 4 substituting 5-(4-methylbenzyl)thiazol-2-amine for 5-phenylthiazol-2-amine and substituting 1-phenyl-1H-pyrazole-3-carboxylic acid for 5-(3-nitrophenyl)furan-2-carboxylic acid.

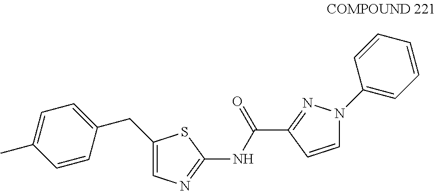 [SMILES: Cc1ccc(Cc2cnc(NC(=O)c3ccn(-c4ccccc4)n3)s2)cc1]

¹H NMR (400 MHz, DMSO-d6) δ 12.22 (s, 1H), 8.67 (d, J=2.6 Hz, 1H), 8.05 (t, J=0.9 Hz, 1H), 8.04-8.00 (m, 1H), 7.59-7.52 (m, 2H), 7.43-7.37 (m, 1H), 7.31 (t, J=1.0 Hz, 1H), 7.21-7.11 (m, 5H), 4.07 (s, 2H), 2.28 (s, 3H); Method 1, retention time: 6.501 min; HRMS: m/z (M+H)⁺=375.1280 (Calculated for C₂₁H₁₉N₄OS=375.1274).

COMPOUND 222 was prepared according to the method described in Scheme 4 substituting 5-(4-fluorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

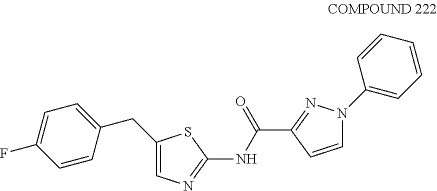 [SMILES: O=C(Nc1ncc(Cc2ccc(F)cc2)s1)c1ccn(-c2ccccc2)n1]

¹H NMR (400 MHz, DMSO-d6) δ 12.25 (s, 1H), 8.67 (d, J=2.6 Hz, 1H), 8.05 (d, J=1.3 Hz, 1H), 8.03 (dd, J=2.0, 0.9 Hz, 1H), 7.59-7.53 (m, 2H), 7.43-7.37 (m, 1H), 7.37-7.31 (m, 3H), 7.19-7.12 (m, 3H), 4.13 (s, 2H); Method 1, retention time: 6.210 min; HRMS: m/z (M+H)⁺=379.1024 (Calculated for C₂₀H₁₆FN₄OS=379.1023).

COMPOUND 223 was prepared according to the method described in Scheme 4 substituting 5-(4-methoxybenzyl)thiazol-2-amine for 5-phenylthiazol-2-amine and substituting 1-phenyl-1H-pyrazole-3-carboxylic acid for 5-(3-nitrophenyl)furan-2-carboxylic acid.

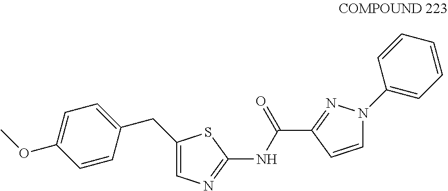 [SMILES: COc1ccc(Cc2cnc(NC(=O)c3ccn(-c4ccccc4)n3)s2)cc1]

¹H NMR (400 MHz, DMSO-d6) δ 12.21 (s, 1H), 8.67 (d, J=2.6 Hz, 1H), 8.05 (d, J=1.3 Hz, 1H), 8.03 (t, J=1.1 Hz, 1H), 7.60-7.52 (m, 2H), 7.43-7.37 (m, 1H), 7.32-7.28 (m, 1H), 7.24-7.19 (m, 2H), 7.16 (d, J=2.6 Hz, 1H), 6.92-6.86 (m, 2H), 4.05 (s, 2H), 3.73 (s, 3H); Method 1, retention time: 6.102 min; HRMS: m/z (M+H)⁺=391.1204 (Calculated for C₂₁H₁₉N₄O₂S=391.1223).

COMPOUND 224 was prepared according to the method described in Scheme 4 substituting 5-(4-chlorobenzyl)thiazol-2-amine for 5-phenylthiazol-2-amine and substituting 1-phenyl-1H-pyrazole-3-carboxylic acid for 5-(3-nitrophenyl)furan-2-carboxylic acid.

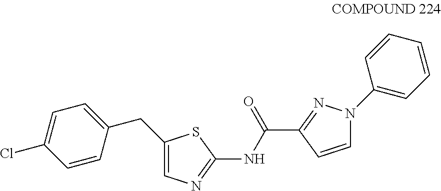 [SMILES: O=C(Nc1ncc(Cc2ccc(Cl)cc2)s1)c1ccn(-c2ccccc2)n1]

¹H NMR (400 MHz, DMSO-d6) δ 12.26 (s, 1H), 8.71-8.61 (m, 1H), 8.05 (d, J=1.3 Hz, 1H), 8.03 (t, J=1.4 Hz, 1H), 7.60-7.52 (m, 2H), 7.45-7.36 (m, 3H), 7.33 (d, J=8.4 Hz, 3H), 7.17 (d, J=2.5 Hz, 1H), 4.14 (s, 2H); Method 1, retention time: 6.529 min; HRMS: m/z (M+H)⁺=395.0728 (Calculated for C₂₀H₁₆ClN₄OS=395.0728).

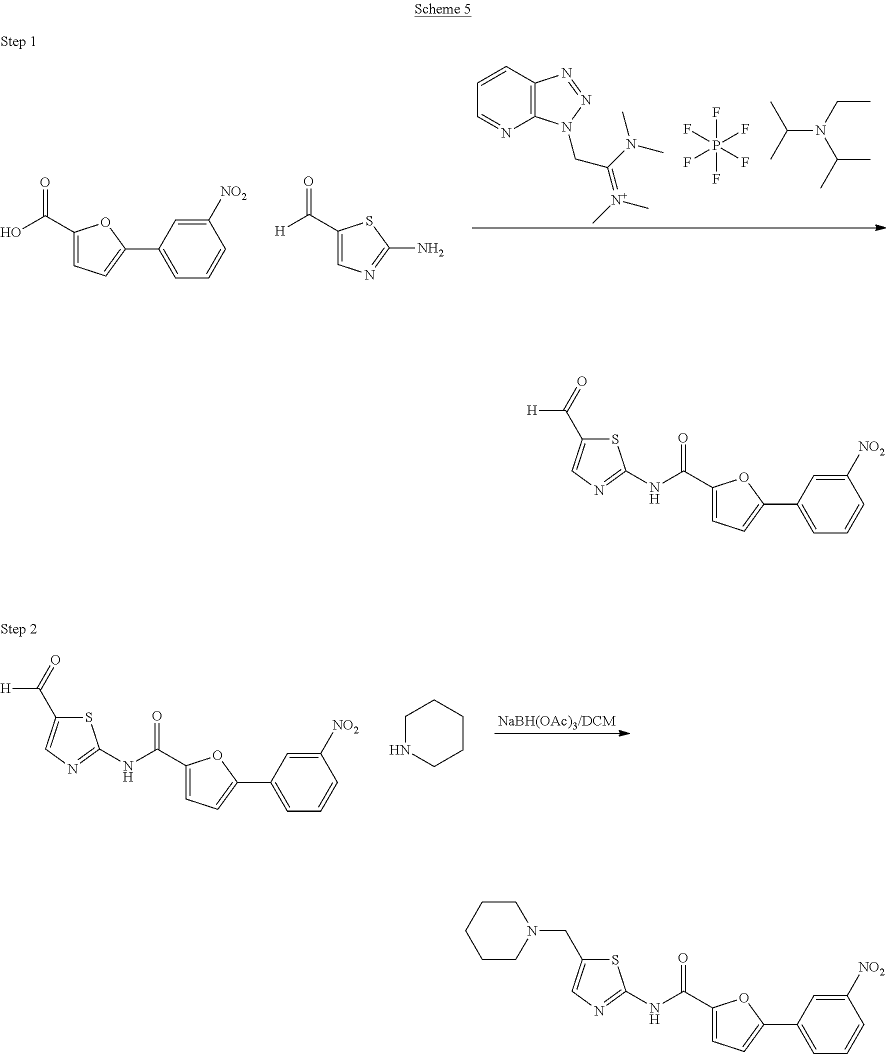

Scheme 5, step 1 was prepared according to the method described in Scheme 4 substituting 2-aminothiazole-5-carbaldehyde for 5-phenylthiazol-2-amine.

### Scheme 5, Step 2

The mixture of N-(5-formylthiazol-2-yl)-5-(3-nitrophenyl)furan-2-carboxamide (30 mg, 0.087 mmol), PIPERIDINE (10.38 μl, 0.105 mmol) and SODIUM TRIACETOXYBOROHYDRIDE (27.8 mg, 0.131 mmol) in DCM (1 ml) was stirred at r.t. for overnight. The crude product was purified by reverse phase chromatography to give COMPOUND 87.

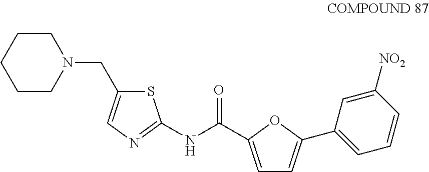 [SMILES: O=C(Nc1ncc(CN2CCCCC2)s1)c1ccc(-c2cccc([N+](=O)[O-])c2)o1]

¹H NMR (400 MHz, DMSO-d6) δ 9.44 (s, 1H), 8.89-8.79 (m, 1H), 8.48 (dt, J=8.0, 1.3 Hz, 1H), 8.26 (ddd, J=8.2, 2.3, 1.0 Hz, 1H), 7.86-7.77 (m, 1H), 7.72 (s, 1H), 7.69 (d, J=3.8 Hz, 1H), 7.53 (d, J=3.7 Hz, 1H), 4.56 (d, J=4.6 Hz, 2H), 3.42 (d, J=12.2 Hz, 2H), 2.88 (dd, J=13.0, 9.1 Hz, 2H), 1.85 (d, J=14.4 Hz, 2H), 1.64 (dt, J=26.9, 14.1 Hz, 4H)); Method 1, retention time: 4.203 min; HRMS: m/z (M+H)⁺=413.1281 (Calculated for C₂₀H₂₁N₄O₄S=413.1278).

COMPOUND 81 was prepared according to the method described in Scheme 5, step 2 substituting morpholine for piperidine.

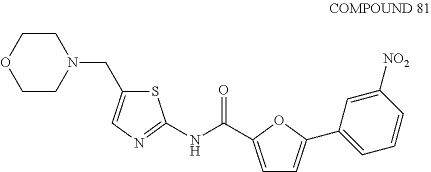 [SMILES: O=C(Nc1ncc(CN2CCOCC2)s1)c1ccc(-c2cccc([N+](=O)[O-])c2)o1]

¹H NMR (400 MHz, DMSO-d6) δ 13.15 (s, 1H), 10.03 (s, 1H), 8.84 (t, J=1.9 Hz, 1H), 8.47 (dt, J=8.1, 1.2 Hz, 1H), 8.26 (ddd, J=8.2, 2.4, 1.0 Hz, 1H), 7.88-7.75 (m, 1H), 7.69 (s, 1H), 7.53 (d, J=3.7 Hz, 1H), 4.63 (s, 2H), 3.99 (s, 2H), 3.62 (s, 4H), 3.11 (s, 2H); Method 1, retention time: 3.991 min; HRMS: m/z (M+H)⁺=415.1054 (Calculated for C₁₉H₁₉N₄O₅S=415.1071).

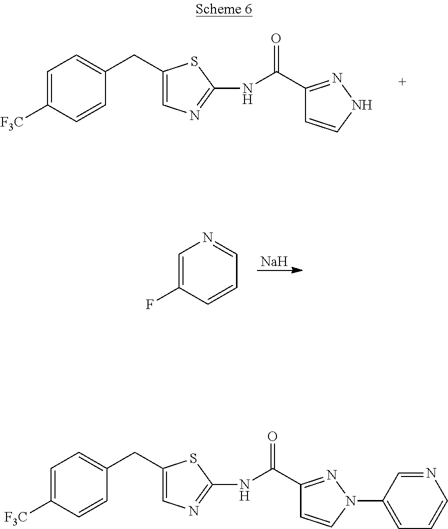

Scheme 6 To a solution of N-(5-(4-(trifluoromethyl)benzyl)thiazol-2-yl)-1H-pyrazole-3-carboxamide (50 mg, 0.14 mmol) in dry DMF (1 mL) was added sodium hydride (12.5 mg, 0.31 mmol). The reaction mixture evolved gas, became purple, and stirred at rt for 15 min. 3-fluoropyridine (0.18 mL, 2.13 mmol) was added and the reaction mixture was heated at 130° C. for 5 hr. The reaction mixture became reddish-brown and was purified by reverse phase chromatography to give COMPOUND 175.

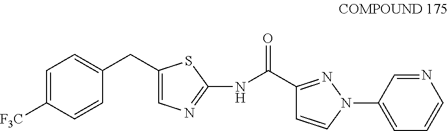 [SMILES: O=C(Nc1ncc(Cc2ccc(C(F)(F)F)cc2)s1)c1ccn(-c2cccnc2)n1]

HRMS: m/z (M+H)⁺=430.0934 (Calculated for C₂₀H₁₅F₃N₅OS=430.0944).

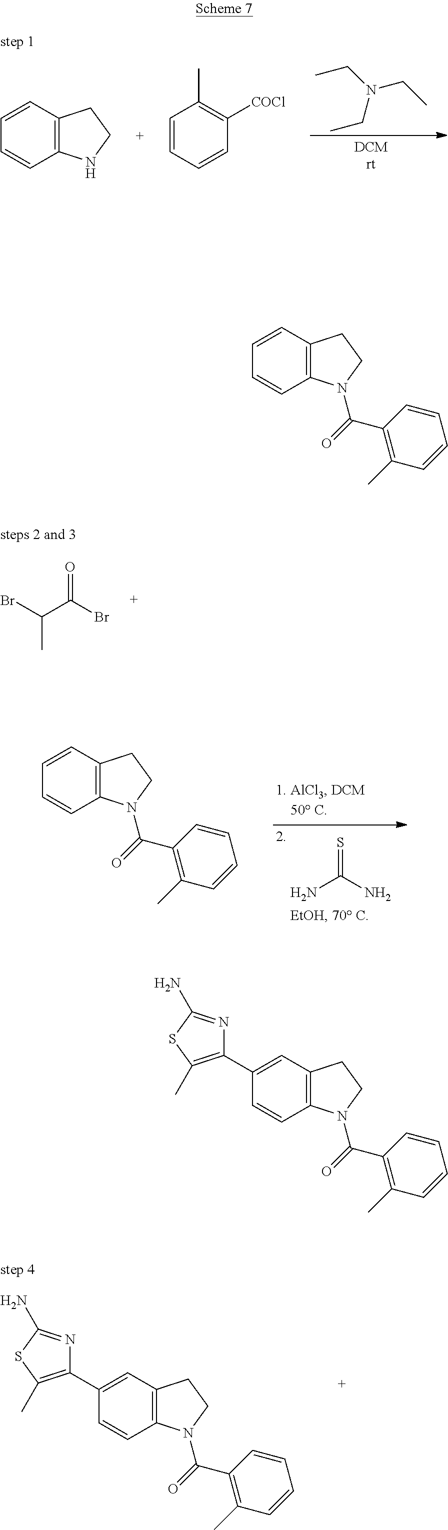

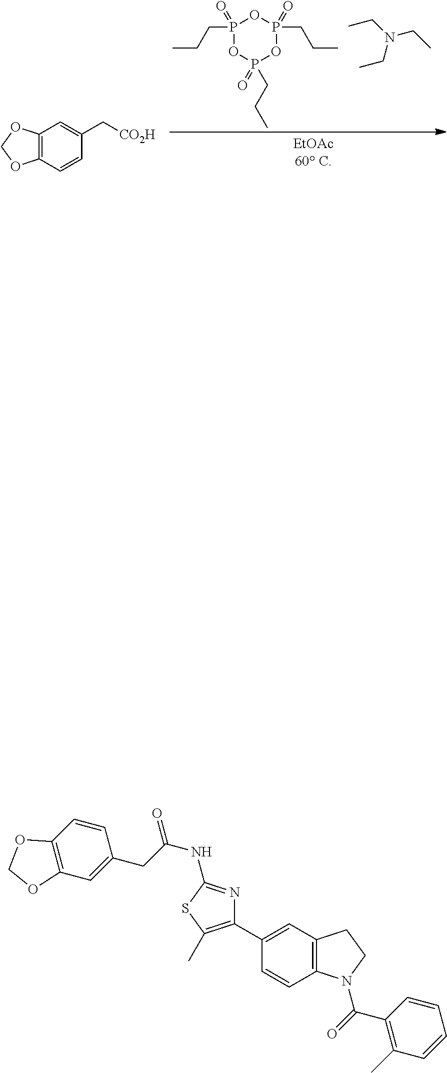

### Scheme 7, Step 1

To a solution of indoline (4.91 mL, 43.8 mmol) and triethylamine (12.21 mL, 88 mmol) in DCM (30 ml) was added 2-methylbenzoyl chloride (6 mL, 46.0 mmol). The reaction became very warm. The reaction mixture stirred at rt overnight. The reaction mixture was diluted with 0.5N NaOH and DCM. The layers were separated and the aqueous layer was reextracted with DCM. The combined organic layers were dried with MgSO₄ and concentrated in vacuo to afford an oil. The residue was taken up in hexanes with a small amount of EtOAc. A precipitate formed. The precipitate was filtered and washed with hexanes. Indolin-1-yl(o-tolyl)methanone (9.17 g, 88%) was isolated as a tan solid and used without further purification; LCMS: m/z (M+H)⁺=238.1.

### Scheme 7, Step 2

To a solution of indolin-1-yl(o-tolyl)methanone (1.5 g, 6.32 mmol) in DCM (20 mL) was added aluminum trichloride (2.53 g, 18.96 mmol) followed by 2-bromopropanoyl bromide (1.986 mL, 18.96 mmol). The resulting reaction mixture was heated at 50° C. for 5 hr. The reaction mixture was cooled to rt and poured onto ice water. The resulting mixture was treated with a saturated aqueous solution of potassium sodium tartrate (Rochelle's salts) and stirred rapidly for 20 min. The mixture was neutralized with 0.5N NaOH. The layers were separated and the aqueous layer was reextracted with DCM. The combined organic layers were dried with MgSO₄ and concentrated in vacuo to afford 2-bromo-1-(1-(2-methylbenzoyl)indolin-5-yl)propan-1-one as a black oil; LCMS: m/z (M+H)+=372.0, 374.0. This material was used in the following step without purification.

### Scheme 7, Step 3

Thiourea (1.203 g, 15.80 mmol) was added to a solution of 2-bromo-1-(1-(2-methylbenzoyl)indolin-5-yl)propan-1-one (6.32 mmol) in EtOH (20 mL). The reaction mixture was heated at 70° C. for 16.5 hr. The reaction mixture was cooled to rt, diluted with water, and basified with ammonium hydroxide. The mixture was diluted with DCM and extracted (2×). The combined organic layers were dried with MgSO₄ and concentrated in vacuo to afford (5-(2-amino-5-methylthiazol-4-yl)indolin-1-yl)(o-tolyl)methanone (2.2 g, quant) as an orange-brown foam; LCMS: m/z (M+H)⁺=350.1. This was used in the following step without additional purification.

### Scheme 7, Step 4

To a solution of 2-(benzo[d][1,3]dioxol-5-yl)acetic acid (452 mg, 2.507 mmol), (5-(2-amino-5-methylthiazol-4-yl)indolin-1-yl)(o-tolyl)methanone (730 mg, 2.089 mmol), and triethylamine (1.456 mL, 10.45 mmol) in EtOAc (10 ml) was added 50 wt. % propylphosphonic anhydride solution in EtOAc (2.5 mL, 4.2 mmol). The mixture was stirred at rt for 5 min and then heated at 60° C. for 5 hr. The reaction mixture was cooled to rt and diluted with water, 0.5N NaOH, and EtOAc. The layers were separated and the aqueous layer was reextracted with EtOAc. The combined organic layers were dried with MgSO₄, and concentrated in vacuo to afford a residue. The residue was taken up in DMSO and subsequently purified by reverse phase chromatography utilizing a gradient of 10 to 100% acetonitrile/water to give Compound 4.

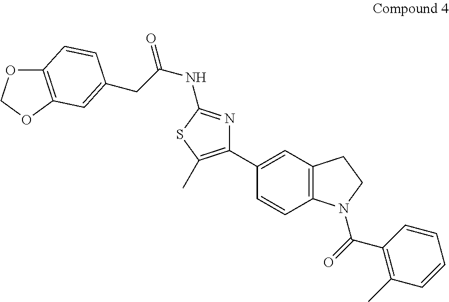 [SMILES: Cc1ccccc1C(=O)N1CCc2cc(-c3nc(NC(=O)Cc4ccc5c(c4)OCO5)sc3C)ccc21]

¹H NMR analysis at room temperature is complicated by amide rotamers that are present as a result of the ortho-methyl group in the indolin-1-yl(o-tolyl)methanone segment of the molecule. ¹H NMR (400 MHz, DMSO-d₆, 75° C.) δ 11.95 (s, 1H), 8.14 (s, 1H), 7.51 (dq, J=1.8, 0.9 Hz, 1H), 7.43-7.14 (m, 5H), 6.94-6.65 (m, 3H), 5.95 (s, 2H), 3.65 (s, 2H), 3.13 (t, J=8.4 Hz, 2H), 3.07 (d, J=0.9 Hz, 2H), 2.42 (s, 3H), 2.26 (s, 3H); ¹³C NMR (101 MHz, DMSO-d₆, 75° C.) δ 169.54, 168.40, 154.19, 147.73, 146.64, 144.50, 141.72, 137.88, 134.02, 133.37, 131.21, 130.86, 129.58, 129.06, 127.33, 126.45, 126.12, 125.11, 122.67, 120.72, 109.98, 109.97, 108.52, 101.27, 49.64, 41.82, 27.75, 18.88, 12.20; LC-MS Retention Time: t₁=6.186 min; HRMS: m/z (M+H)⁺=512.1628 (Calculated for C₂₉H₂₆N₃O₄S=512.1639).

Compound 7 was prepared according to the method described in Scheme 7, step 4 substituting 2-(4-methoxyphenyl)acetic acid for 2-(benzo[d][1,3]dioxol-5-yl)acetic acid.

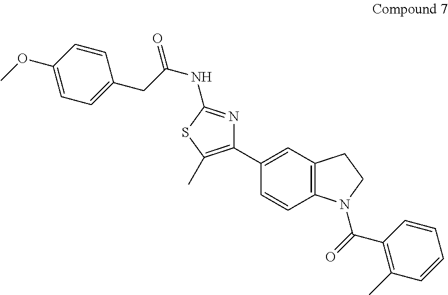 [SMILES: COc1ccc(CC(=O)Nc2nc(-c3ccc4c(c3)CCN4C(=O)c3ccccc3C)c(C)s2)cc1]

HRMS: m/z (M+H)⁺=498.1846 (Calculated for C₂₉H₂₈N₃O₃S=498.1846); ¹H NMR analysis at room temperature is complicated by amide rotamers that are present as a result of the ortho-methyl group in the indolin-1-yl(o-tolyl)methanone segment of the molecule.

Compound 6 was prepared according to the method described in Scheme 7, step 4 substituting 2-(3-methoxyphenyl)acetic acid for 2-(benzo[d][1,3]dioxol-5-yl)acetic acid.

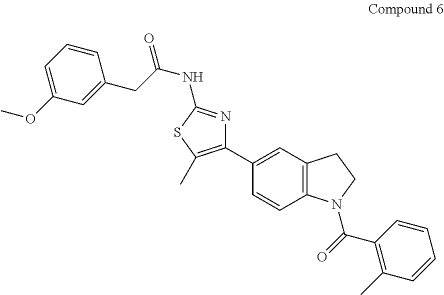 [SMILES: COc1cccc(CC(=O)Nc2nc(-c3ccc4c(c3)CCN4C(=O)c3ccccc3C)c(C)s2)c1]

HRMS: m/z (M+H)⁺=498.1836 (Calculated for C₂₉H₂₈N₃O₃S=498.1846); ¹H NMR analysis at room temperature is complicated by amide rotamers that are present as a result of the ortho-methyl group in the indolin-1-yl(o-tolyl)methanone segment of the molecule.

Compound 8 was prepared according to the method described in Scheme 7, step 4 substituting 2-phenylacetic acid for 2-(benzo[d][1,3]dioxol-5-yl)acetic acid.

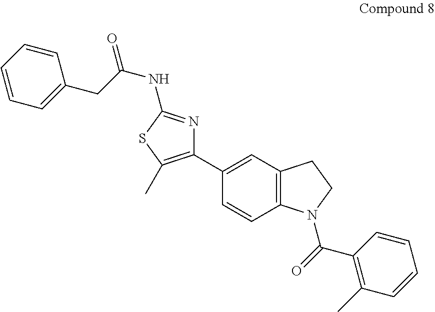 [SMILES: Cc1ccccc1C(=O)N1CCc2cc(-c3nc(NC(=O)Cc4ccccc4)sc3C)ccc21]

HRMS: m/z (M+H)⁺=468.1733 (Calculated for C₂₈H₂₆N₃O₂S=468.1740); ¹H NMR analysis at room temperature is complicated by amide rotamers that are present as a result of the ortho-methyl group in the indolin-1-yl(o-tolyl)methanone segment of the molecule.

Compound 9 was prepared according to the method described in Scheme 7, step 4 substituting 2-(naphthalen-2-yl)acetic acid for 2-(benzo[d][1,3]dioxol-5-yl)acetic acid.

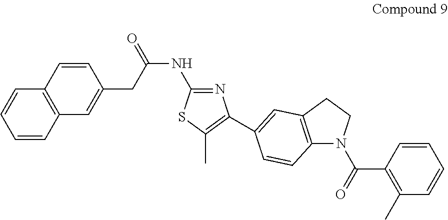 [SMILES: Cc1ccccc1C(=O)N1CCc2cc(-c3nc(NC(=O)Cc4ccc5ccccc5c4)sc3C)ccc21]

HRMS: m/z (M+H)⁺=518.1888 (Calculated for C₃₂H₂₈N₃O₂S=518.1897); ¹H NMR analysis at room temperature is complicated by amide rotamers that are present as a result of the ortho-methyl group in the indolin-1-yl(o-tolyl)methanone segment of the molecule.

Compound 12 was prepared according to the method described in Scheme 7, step 4 substituting 2-(3,4-difluorophenyl)acetic acid for 2-(benzo[d][1,3]dioxol-5-yl)acetic acid.

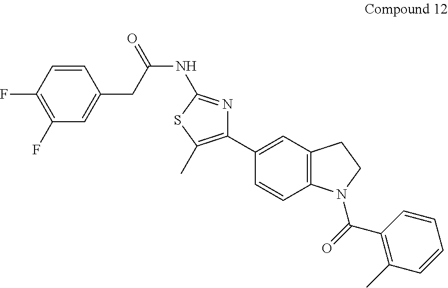 [SMILES: Cc1ccccc1C(=O)N1CCc2cc(-c3nc(NC(=O)Cc4ccc(F)c(F)c4)sc3C)ccc21]

HRMS: m/z (M+H)⁺=504.1540 (Calculated for C₂₈H₂₄F₂N₃O₂S=504.1552); ¹H NMR analysis at room temperature is complicated by amide rotamers that are present as a result of the ortho-methyl group in the indolin-1-yl(o-tolyl)methanone segment of the molecule.

Compound 10 was prepared according to the method described in Scheme 7, step 4 substituting 2-(3,4-dimethylphenyl)acetic acid for 2-(benzo[d][1,3]dioxol-5-yl)acetic acid.

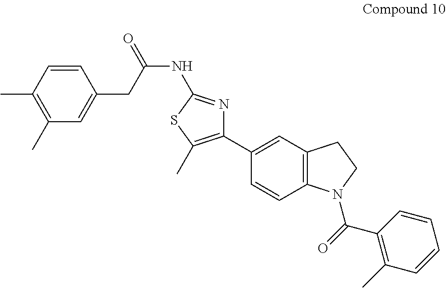 [SMILES: Cc1ccc(CC(=O)Nc2nc(-c3ccc4c(c3)CCN4C(=O)c3ccccc3C)c(C)s2)cc1C]

HRMS: m/z (M+H)⁺=496.2047 (Calculated for C₃₀H₃₀N₃O₂S=496.2053); ¹H NMR analysis at room temperature is complicated by amide rotamers that are present as a result of the ortho-methyl group in the indolin-1-yl(o-tolyl)methanone segment of the molecule.

Compound 5 was prepared according to the method described in Scheme 7, step 4 substituting 2-(2,3-dihydrobenzo[b][1,4]dioxin-6-yl)acetic acid for 2-(benzo[d][1,3]dioxol-5-yl)acetic acid.

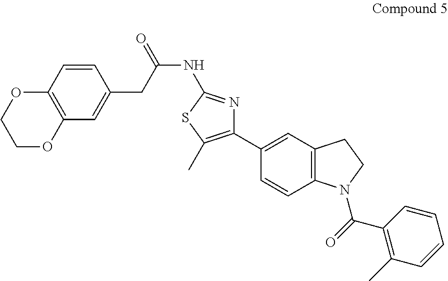 [SMILES: Cc1ccccc1C(=O)N1CCc2cc(-c3nc(NC(=O)Cc4ccc5c(c4)OCCO5)sc3C)ccc21]

HRMS: m/z (M+H)⁺=526.1797 (Calculated for C₃₀H₂₈N₃O₄S=526.1795); ¹H NMR analysis at room temperature is complicated by amide rotamers that are present as a result of the ortho-methyl group in the indolin-1-yl(o-tolyl)methanone segment of the molecule.

Compound 13 was prepared according to the method described in Scheme 7, step 4 substituting benzo[d][1,3]dioxole-5-carboxylic acid for 2-(benzo[d][1,3]dioxol-5-yl)acetic acid and heating at 70° C. overnight.

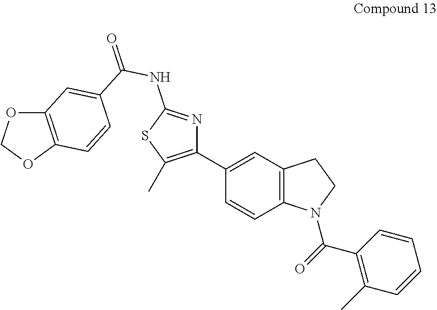 [SMILES: Cc1ccccc1C(=O)N1CCc2cc(-c3nc(NC(=O)c4ccc5c(c4)OCO5)sc3C)ccc21]

HRMS: m/z (M+H)⁺=498.1471 (Calculated for C₂₈H₂₄N₃O₄S=498.1482); ¹H NMR analysis at room temperature is complicated by amide rotamers that are present as a result of the ortho-methyl group in the indolin-1-yl(o-tolyl)methanone segment of the molecule.

Compound 2 was prepared according to the methods described in Scheme 7, steps 2-4 substituting 2-bromoacetyl bromide for 2-bromopropanoyl bromide in step 2,2-bromo-1-(1-(2-methylbenzoyl)indolin-5-yl)ethanone for 2-bromo-1-(1-(2-methylbenzoyl)indolin-5-yl)propan-1-one in step 3, and (5-(2-amino-5-methylthiazol-4-yl)indolin-1-yl)(o-tolyl)methanone for (5-(2-aminothiazol-4-yl)indolin-1-yl)(o-tolyl)methanone in step 4.

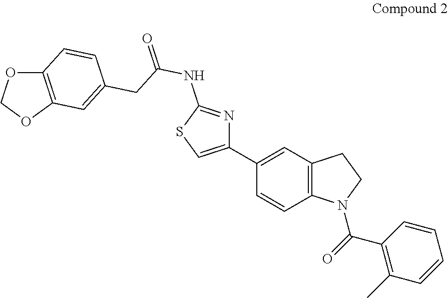 [SMILES: Cc1ccccc1C(=O)N1CCc2cc(-c3csc(NC(=O)Cc4ccc5c(c4)OCO5)n3)ccc21]

HRMS: m/z (M+H)⁺=498.1488 (Calculated for C₂₈H₂₄N₃O₄S=498.1482); ¹H NMR analysis at room temperature is complicated by amide rotamers that are present as a result of the ortho-methyl group in the indolin-1-yl(o-tolyl)methanone segment of the molecule.

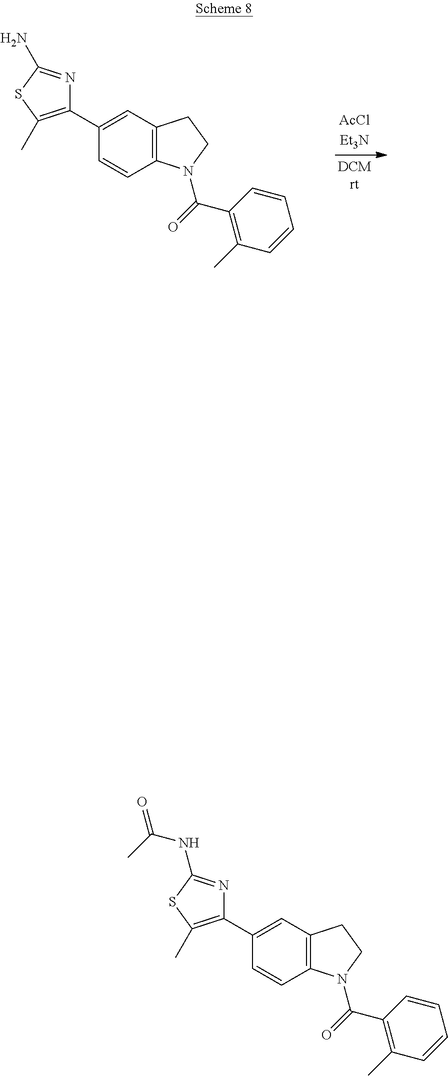 [SMILES: Cc1ccccc1C(=O)N1CCc2cc(-c3nc(N)sc3C)ccc21 ; CC(=O)Nc1nc(-c2ccc3c(c2)CCN3C(=O)c2ccccc2C)c(C)s1]

Scheme 8 To a solution of (5-(2-amino-5-methylthiazol-4-yl)indolin-1-yl)(o-tolyl)methanone (55 mg, 0.157 mmol) and triethylamine (0.066 mL, 0.472 mmol) in DCM (2 ml) was added acetyl chloride (0.022 mL, 0.315 mmol). The reaction stirred at rt for 40 min. By LCMS, a significant amount of bis-acylated product was evident. The reaction mixture was diluted with 0.5N NaOH and extracted with DCM (3×). The combined organic layers were dried with MgSO₄ and concentrated in vacuo to afford a residue which was taken up in DMSO and subsequently purified by reverse phase chromatography to give Compound 11.

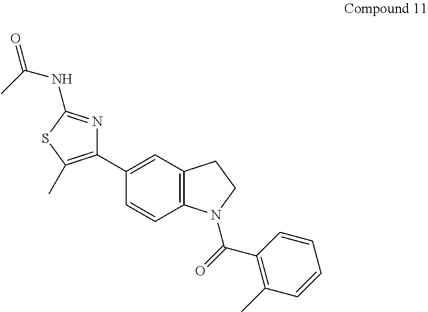 [SMILES: CC(=O)Nc1nc(-c2ccc3c(c2)CCN3C(=O)c2ccccc2C)c(C)s1]

HRMS: m/z (M+H)⁺=392.1417 (Calculated for C₂₂H₂₂N₃O₂S=392.1427); ¹H NMR analysis at room temperature is complicated by amide rotamers that are present as a result of the ortho-methyl group in the indolin-1-yl(o-tolyl)methanone segment of the molecule.

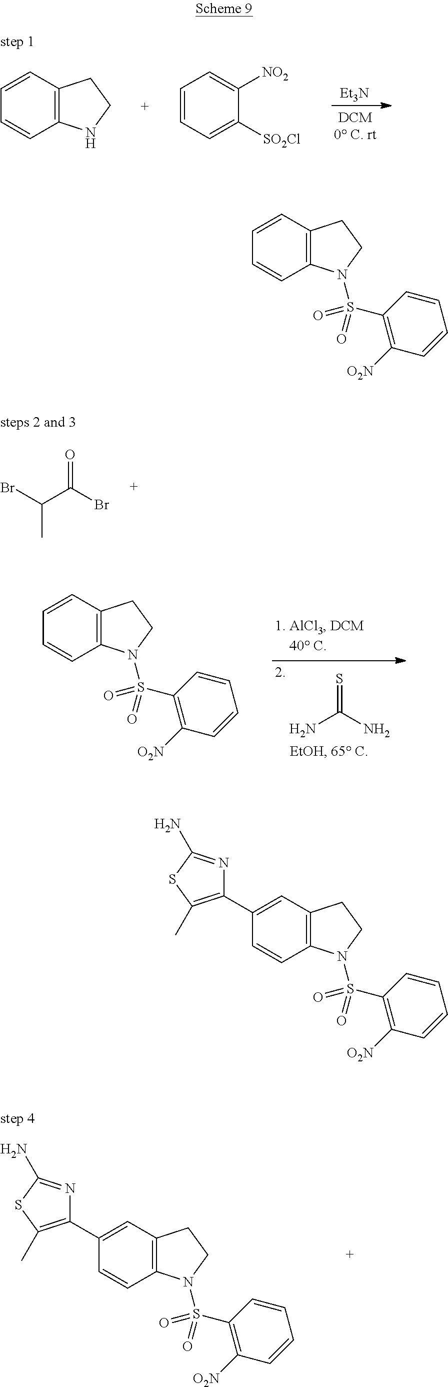

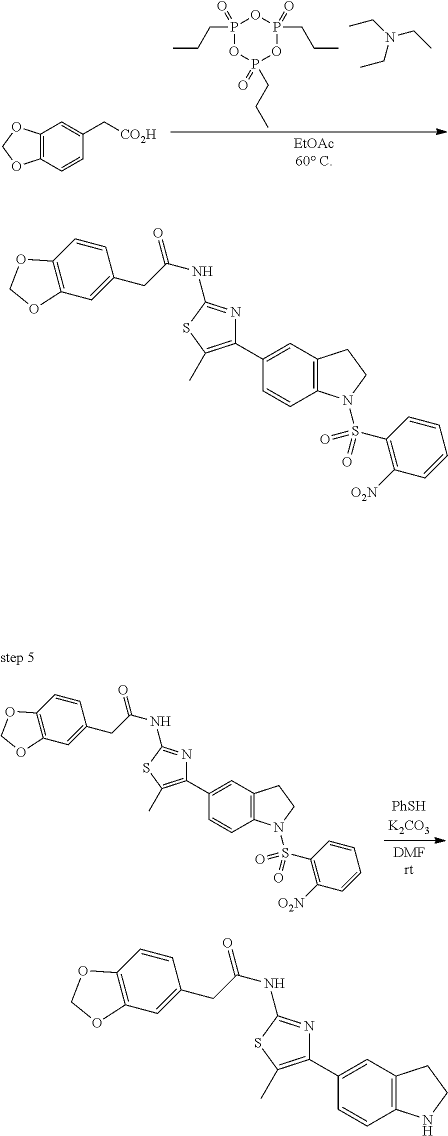

### Scheme 9, Step 1

To an ice-cooled solution of indoline (0.5 mL, 4.46 mmol) and triethylamine (1.24 mL, 8.9 mmol) in DCM (5 ml) was added 2-nitrobenzene-1-sulfonyl chloride (1.04 g, 4.68 mmol). The reaction became yellow and a solid formed shortly after the addition. The reaction mixture slowly warmed to rt and was stirred overnight. The reaction mixture was diluted with water and DCM. The layers were separated and the aqueous layer was reextracted with DCM. The combined organic layers were dried with MgSO₄ and concentrated in vacuo to afford 1-((2-nitrophenyl)sulfonyl)indoline as a solid; LCMS: m/z (M+H)⁺=305.1. Assumed quantitative conversion and used without purification in step 2 of Scheme 7.

### Scheme 9, Step 2

To a solution of 1-((2-nitrophenyl)sulfonyl)indoline (4.46 mmol) in DCM (12 mL) was added aluminum trichloride (1.78 g, 13.4 mmol) followed by 2-bromopropanoyl bromide (1.4 mL, 13.4 mmol). The resulting reaction mixture was heated at 40° C. for 5 hr. The reaction mixture was cooled to rt and poured onto ice water. The resulting mixture was treated with a saturated aqueous solution of potassium sodium tartrate (Rochelle's salts) and stirred rapidly for 20 min. The mixture was neutralized with 0.5N NaOH. The layers were separated and the aqueous layer was reextracted with DCM. The combined organic layers were dried with MgSO₄ and concentrated in vacuo to afford 2-bromo-1-(1-((2-nitrophenyl)sulfonyl)indolin-5-yl)propan-1-one as a brown oil; LCMS: m/z (M+H)⁺=439.0, 374.0. This material was used in the following step without purification.

### Scheme 9, Step 3

Thiourea (0.85 g, 11.2 mmol) was added to a solution of 2-bromo-1-(1-((2-nitrophenyl)sulfonyl)indolin-5-yl)propan-1-one (4.46 mmol) in EtOH (12 mL). The reaction mixture was heated at 65° C. for 15 hr. The reaction mixture was cooled to rt, diluted with water and DCM and extracted (3×). The combined organic layers were dried with MgSO₄ and concentrated in vacuo to afford 5-methyl-4-(1-((2-nitrophenyl)sulfonyl)indolin-5-yl)thiazol-2-amine as a solid; LCMS: m/z (M+H)=417.1. Assumed quantitative conversion and used without purification in step 4 of Scheme 7.

### Scheme 9, Step 4

To a solution of 2-(benzo[d][1,3]dioxol-5-yl)acetic acid (964 mg, 5.35 mmol), 5-methyl-4-(1-((2-nitrophenyl)sulfonyl)indolin-5-yl)thiazol-2-amine (4.46 mmol), and triethylamine (3.11 mL, 22.3 mmol) in EtOAc (20 ml) was added 50 wt. % propylphosphonic anhydride solution in EtOAc (4.6 mL, 8.9 mmol). The mixture was stirred at rt for 5 min and then heated at 60° C. for 5 hr. The reaction mixture was cooled to rt and diluted with water and EtOAc. The layers were separated and the aqueous layer was reextracted with EtOAc. The combined organic layers were dried with MgSO₄ and concentrated in vacuo to afford 2-(benzo[d][1,3]dioxol-5-yl)-N-(5-methyl-4-(1-((2-nitrophenyl)sulfonyl)indolin-5-yl)thiazol-2-yl)acetamide (2.53 g, ˜98% over 4 steps) as a solid; LCMS: m/z (M+H)⁺=579.1. This material was used without purification in step 5 of Scheme 7.

### Scheme 9, Step 5

To a mixture of 2-(benzo[d][1,3]dioxol-5-yl)-N-(5-methyl-4-(1-((2-nitrophenyl)sulfonyl)indolin-5-yl)thiazol-2-yl)acetamide (2.5 g, 4.32 mmol) and potassium carbonate (2.4 g, 17 mmol) in DMF (10 ml) was added thiophenol (0.89 mL, 8.6 mmol). The mixture was stirred at rt for 3.5 hr. The reaction mixture was diluted with water, sat. aq. sodium bicarbonate, and EtOAc. The layers were separated and the aqueous layer was reextracted with EtOAc (this presented problems as the product has poor solubility). The combined organic layers were dried with MgSO₄, concentrated in vacuo, and purified by silica gel chromatography. Chromatography proved challenging as the product precipitated on the column despite dry loading. Elution of product began with a gradient of 20 to 100% EtOAc/hexanes, however given product precipitation, it required 10% methanol/DCM to complete. In the future, attempt precipitation/recrystallization for purification. 2-(Benzo[d][1,3]dioxol-5-yl)-N-(4-(indolin-5-yl)-5-methylthiazol-2-yl)acetamide (Compound 17) (1.26 g, 58%) was isolated as a solid.

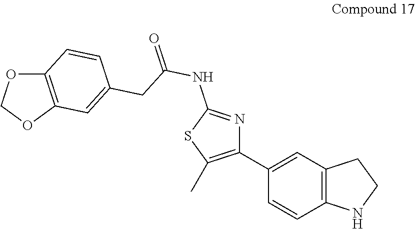 [SMILES: Cc1sc(NC(=O)Cc2ccc3c(c2)OCO3)nc1-c1ccc2c(c1)CCN2]

¹H NMR (400 MHz, DMSO-d6) δ ppm 12.18 (s, 1H), 7.49-7.43 (m, 1H), 7.41-7.33 (m, 1H), 6.94 (d, J=8.2 Hz, 1H), 6.91-6.80 (m, 2H), 6.76 (dd, J=7.9, 1.7 Hz, 1H), 5.97 (s, 2H), 3.63 (s, 2H), 3.58 (t, J=8.2 Hz, 2H), 3.07 (t, J=8.2 Hz, 2H), 2.40 (s, 3H); HRMS: m/z (M+H)⁺=394.1226 (Calculated for C₂₁H₂₀N₃O₃S=394.1220).

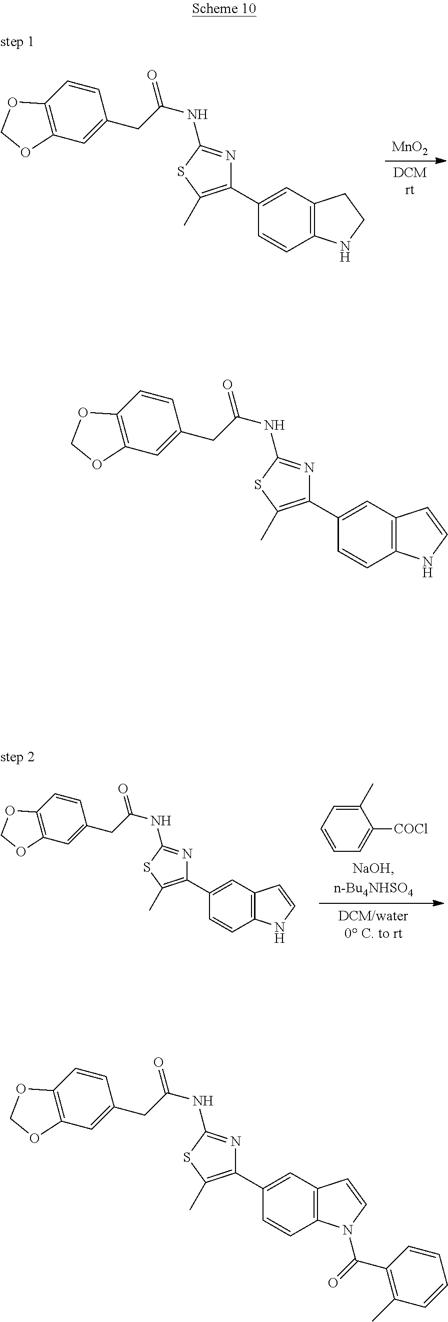

### Scheme 10, Step 1

To a solution of 2-(benzo[d][1,3]dioxol-5-yl)-N-(4-(indolin-5-yl)-5-methylthiazol-2-yl)acetamide (Compound 17) (100 mg, 0.254 mmol) in DCM (6 ml) was added manganese dioxide (221 mg, 2.54 mmol). The reaction mixture stirred at rt for 2.5 hr. The reaction mixture was filtered washing with EtOAc, and the filtrate was concentrated in vacuo to yield N-(4-(1H-indol-5-yl)-5-methylthiazol-2-yl)-2-(benzo[d][1,3]dioxol-5-yl)acetamide as a brown oil; LCMS: m/z (M+H)⁺=392.1. This material was used without purification.

### Scheme 10, Step 2

A solution of 2-methylbenzoyl chloride (22 μL, 165 mmol) in DCM (1 mL) was slowly to an ice-cooled mixture of N-(4-(1H-indol-5-yl)-5-methylthiazol-2-yl)-2-(benzo[d][1,3]dioxol-5-yl)acetamide (50 mg, 0.127 mmol), tetrabutylammonium hydrogen sulfate (5 mg, 0.015 mmol), and sodium hydroxide (15 mg, 0.381 mmol) in DCM (3 ml). The reaction stirred at 0° C. for 20 min and at rt for 20 min. By LCMS, there was virtually no reaction. A second batch of both sodium hydroxide and tetrabutylammonium hydrogen sulfate were added in addition to 2 drops of water. The reaction stirred at rt 2.5 hr. Another batch of 2-methylbenzoyl chloride (10 μL) was added and stirring resumed for 45 min. The reaction mixture was diluted with water and extracted with DCM (3×). The combined organic layers were dried with MgSO₄ and concentrated in vacuo to afford a residue which was taken up in DMF and subsequently purified by reverse phase chromatography to give Compound 30.

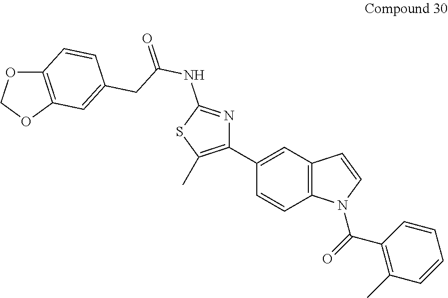 [SMILES: Cc1ccccc1C(=O)n1ccc2cc(-c3nc(NC(=O)Cc4ccc5c(c4)OCO5)sc3C)ccc21]

¹H NMR (400 MHz, DMSO-d6) δ ppm 12.60 (s, 1H), 8.60 (d, J=8.6 Hz, 1H), 8.20 (dd, J=1.8, 0.7 Hz, 1H), 7.97 (dd, J=8.6, 1.8 Hz, 1H), 7.84-7.79 (m, 1H), 7.77-7.65 (m, 2H), 7.41 (d, J=3.7 Hz, 1H), 7.21 (d, J=1.7 Hz, 1H), 7.16 (d, J=7.9 Hz, 1H), 7.12-7.06 (m, 2H), 6.28 (s, 2H), 3.95 (s, 2H), 2.56 (s, 3H), 1 CH₃ signal under DMSO peak; HRMS: m/z (M+H)⁺=510.1471 (Calculated for C₂₉H₂₄N₃O₄S=510.1482).

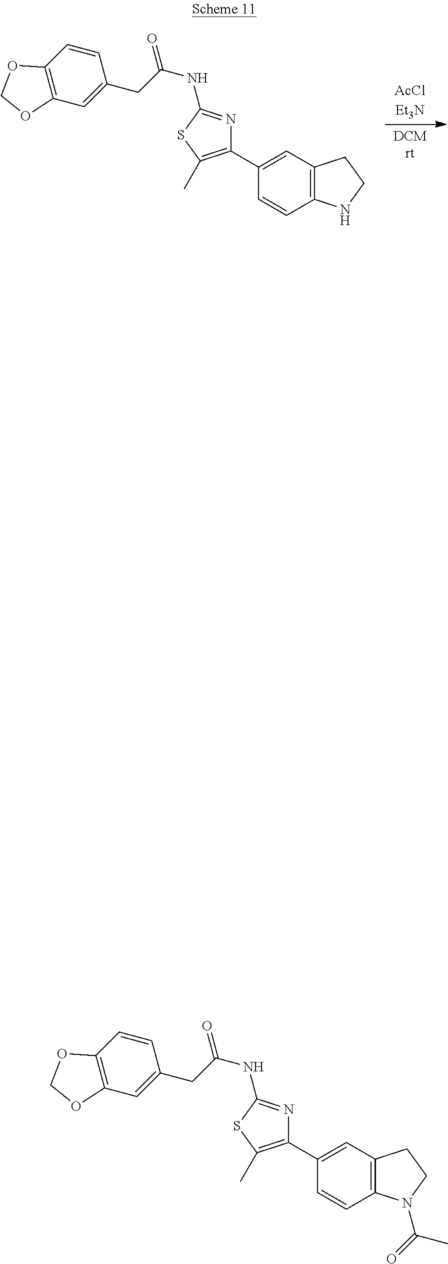

Scheme 11 To a solution of 2-(benzo[d][1,3]dioxol-5-yl)-N-(4-(indolin-5-yl)-5-methylthiazol-2-yl)acetamide (50 mg, 0.127 mmol) and triethylamine (35 μL, 0.254 mmol) in DCM (3 ml) was added acetyl chloride (10 μL, 0.140 mmol). The reaction stirred at rt for 4 hr. The reaction mixture was concentrated under a stream of air to afford a residue which was taken up in DMSO and subsequently purified by reverse phase chromatography to give Compound 21.

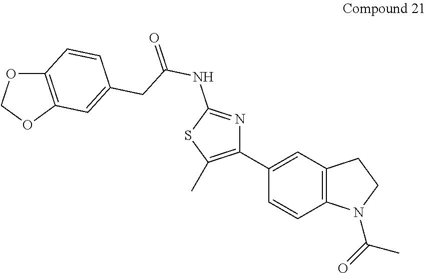 [SMILES: CC(=O)N1CCc2cc(-c3nc(NC(=O)Cc4ccc5c(c4)OCO5)sc3C)ccc21]

¹H NMR (400 MHz, DMSO-d6) δ ppm 12.50 (s, 1H), 8.34 (d, J=8.4 Hz, 1H), 7.77-7.66 (m, 2H), 7.19-7.09 (m, 2H), 7.04 (dd, J=7.9, 1.7 Hz, 1H), 6.24 (s, 2H), 4.39 (t, J=8.5 Hz, 2H), 3.90 (s, 2H), 3.49-3.40 (m, 2H), 2.69 (s, 3H), 2.43 (s, 3H); HRMS: m/z (M+H)⁺=436.1338 (Calculated for C₂₃H₂₂N₃O₄S=436.1326).

Compound 23 was prepared according to the method described in Scheme 11 substituting 4-methylbenzoyl chloride for acetyl chloride. Also, purification was performed by precipitation from DMSO/water mixture.

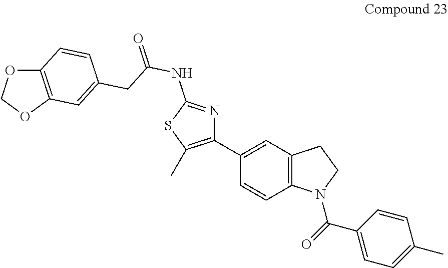 [SMILES: Cc1ccc(C(=O)N2CCc3cc(-c4nc(NC(=O)Cc5ccc6c(c5)OCO6)sc4C)ccc32)cc1]

¹H NMR (400 MHz, DMSO-d6) δ ppm 12.23 (s, 1H), 7.54-7.41 (m, 5H), 7.33-7.25 (m, 2H), 6.91-6.81 (m, 2H), 6.77 (dd, J=8.0, 1.7 Hz, 1H), 5.97 (s, 2H), 4.08-3.96 (m, 2H), 3.63 (s, 2H), 3.11 (t, J=8.3 Hz, 2H), 2.42 (s, 3H), 2.36 (s, 3H); HRMS: m/z (M+H)⁺=512.1640 (Calculated for C₂₉H₂₆N₃O₄S=512.1639).

Compound 25 was prepared according to the method described in Scheme 11 substituting cyclohexylcarbonyl chloride for acetyl chloride.

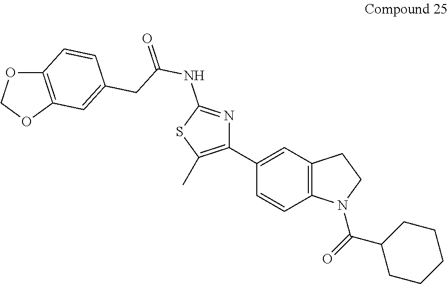 [SMILES: Cc1sc(NC(=O)Cc2ccc3c(c2)OCO3)nc1-c1ccc2c(c1)CCN2C(=O)C1CCCCC1]

¹H NMR (400 MHz, DMSO-d6) δ ppm 12.22 (s, 1H), 8.12 (d, J=8.4 Hz, 1H), 7.49-7.37 (m, 2H), 6.91-6.81 (m, 2H), 6.76 (dd, J=7.9, 1.7 Hz, 1H), 5.97 (s, 2H), 4.18 (t, J=8.4 Hz, 2H), 3.62 (s, 2H), 3.20-3.13 (m, 3H), 2.41 (s, 3H), 1.83-1.61 (m, 5H), 1.47-1.18 (m, 5H); HRMS: m/z (M+H)⁺=504.1960 (Calculated for C₂₈H₃₀N₃O₄S=504.1952).

Compound 14 was prepared according to the method described in Scheme 11 substituting benzoyl chloride for acetyl chloride.

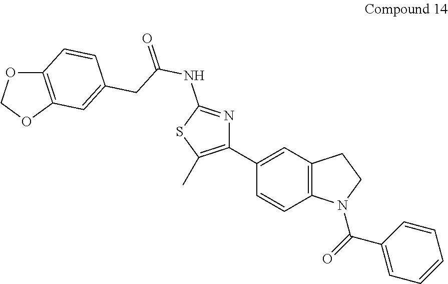 [SMILES: Cc1sc(NC(=O)Cc2ccc3c(c2)OCO3)nc1-c1ccc2c(c1)CCN2C(=O)c1ccccc1]

¹H NMR (400 MHz, DMSO-d6) δ ppm 12.24 (s, 1H), 7.58 (dd, J=7.4, 2.0 Hz, 2H), 7.56-7.44 (m, 6H), 6.91-6.81 (m, 2H), 6.77 (dd, J=8.0, 1.7 Hz, 1H), 5.97 (s, 2H), 4.07-3.96 (m, 2H), 3.63 (s, 2H), 3.18-3.07 (m, 2H), 2.43 (s, 3H); HRMS: m/z (M+H)⁺=498.1487 (Calculated for C₂₈H₂₄N₃O₄S=498.1482).

Compound 29 was prepared according to the method described in Scheme 11 substituting 3-methylbenzoyl chloride for acetyl chloride.

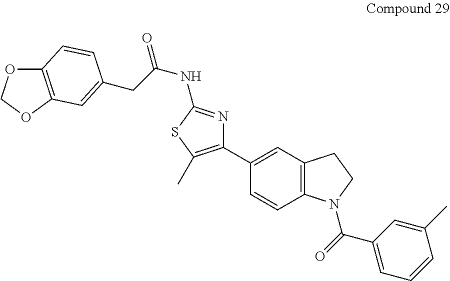 [SMILES: Cc1cccc(C(=O)N2CCc3cc(-c4nc(NC(=O)Cc5ccc6c(c5)OCO6)sc4C)ccc32)c1]

HRMS: m/z (M+H)⁺=512.1630 (Calculated for C₂₉H₂₆N₃O₄S=512.1639).

Compound 24 was prepared according to the method described in Scheme 11 substituting 2-methylbenzene-1-sulfonyl chloride for acetyl chloride.

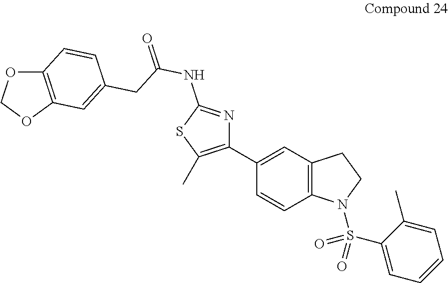 [SMILES: Cc1ccccc1S(=O)(=O)N1CCc2cc(-c3nc(NC(=O)Cc4ccc5c(c4)OCO5)sc3C)ccc21]

¹H NMR (400 MHz, DMSO-d6) δ ppm 12.73 (s, 1H), 8.38 (dd, J=8.0, 1.4 Hz, 1H), 8.09 (td, J=7.5, 1.4 Hz, 1H), 8.03-7.88 (m, 4H), 7.79 (dd, J=8.4, 0.5 Hz, 1H), 7.44-7.34 (m, 2H), 7.29 (dd, J=8.0, 1.7 Hz, 1H), 6.50 (s, 2H), 4.53 (dd, J=8.9, 8.1 Hz, 2H), 4.15 (s, 2H), 3.65-3.56 (m, 2H), 3.06 (s, 3H), 2.92 (s, 3H); HRMS: m/z (M+H)⁺=548.1306 (Calculated for C₂₈H₂₆N₃O₅S₂=548.1308).

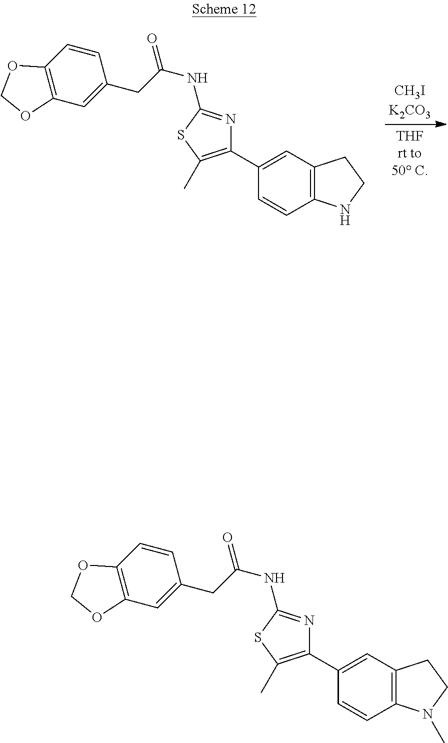 [SMILES: Cc1sc(NC(=O)Cc2ccc3c(c2)OCO3)nc1-c1ccc2c(c1)CCN2 ; "CH3IK2CO3" ; Cc1sc(NC(=O)Cc2ccc3c(c2)OCO3)nc1-c1ccc2c(c1)CCN2C]

### Scheme 12

To a solution of 2-(benzo[d][1,3]dioxol-5-yl)-N-(4-(indolin-5-yl)-5-methylthiazol-2-yl)acetamide (50 mg, 0.127 mmol) and potassium carbonate (35 mg, 0.254 mmol) in THF (3 ml) was added iodomethane (9 μL, 0.140 mmol). The reaction stirred at rt for 4 hr. There was little to no reaction by LCMS analysis. The reaction mixture was heated to 50° C. for 1 hr. An additional aliquot of iodomethane (20 μL, 0.310 mmol) was added and heating resumed overnight (it is likely that ultimately the reaction was no longer being driven to desired product upon heating overnight, but product was likely being driven to over- or bis-alkylation). The reaction mixture was concentrated under a stream of air to afford a residue which was taken up in DMSO and subsequently purified by reverse phase chromatography to give Compound 26.

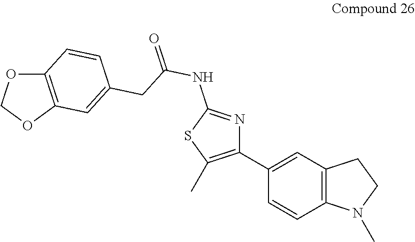 [SMILES: Cc1sc(NC(=O)Cc2ccc3c(c2)OCO3)nc1-c1ccc2c(c1)CCN2C]

¹H NMR (400 MHz, DMSO-d6) δ ppm 12.02 (s, 1H), 7.20-7.12 (m, 2H), 6.80-6.69 (m, 2H), 6.64 (dd, J=8.0, 1.7 Hz, 1H), 6.41 (d, J=8.1 Hz, 1H), 5.85 (s, 2H), 3.50 (s, 2H), 3.16 (t, J=8.2 Hz, 2H), 2.78 (t, J=8.2 Hz, 2H), 2.60 (s, 3H), 2.26 (s, 3H); HRMS: m/z (M+H)⁺=408.1362 (Calculated for C₂₂H₂₂N₃O₃S=408.1376).

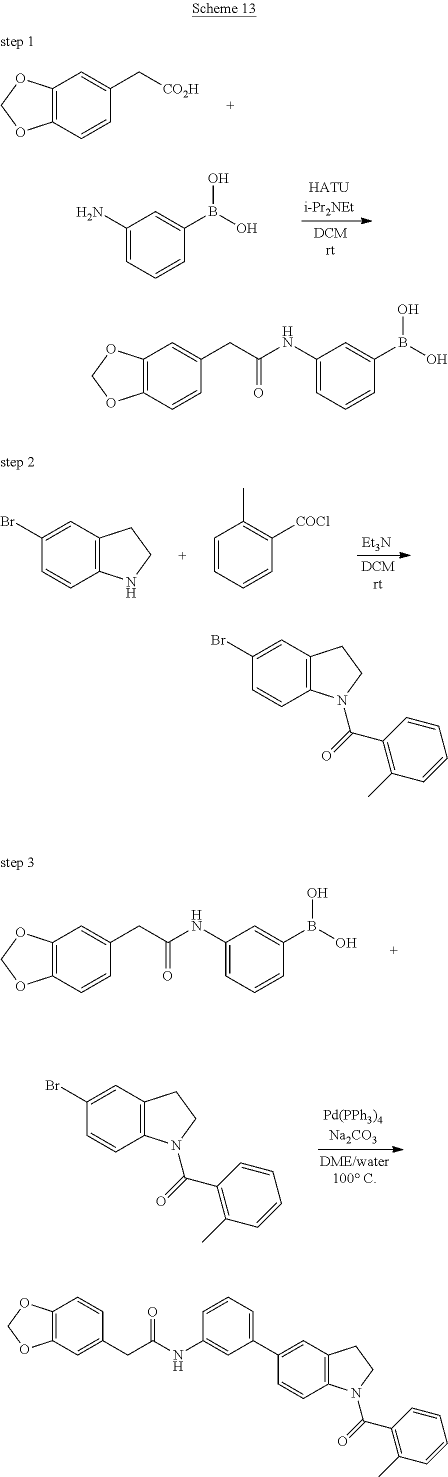

### Scheme 13, Step 1

To a solution of 2-(benzo[d][1,3]dioxol-5-yl)acetic acid (250 mg, 1.388 mmol) and (3-aminophenyl)boronic acid (190 mg, 1.388 mmol) in DCM (5 mL) were added HATU (739 mg, 1.943 mmol) and N,N-diisopropylethylamine (0.727 mL, 4.16 mmol). The reaction stirred at rt for 5 hr. The reaction mixture was diluted with water and extracted with DCM (2×). The combined organic layers were dried with MgSO₄ and concentrated in vacuo to afford (3-(2-(benzo[d][1,3]dioxol-5-yl)acetamido)phenyl)boronic acid, which was used in step 3 of Scheme 11 without purification; LCMS: m/z (M+H)⁺=300.1.

### Scheme 13, Step 2

To an ice-cooled solution of 5-bromoindoline (578 mg, 2.92 mmol) and triethylamine (800 μL, 5.8 mmol) in DCM (5 ml) was added 2-methylbenzoyl chloride (0.4 mL, 3.1 mmol). The reaction mixture was allowed to warm to rt and stirred at rt overnight. A precipitate formed. The reaction mixture was diluted with 0.5N NaOH and DCM. The layers were separated and the aqueous layer was reextracted with DCM. The combined organic layers were dried with MgSO₄ and concentrated in vacuo to afford a residue which was purified by silica gel chromatography (gradient 0 to 30% EtOAc/hexanes). (5-Bromoindolin-1-yl)(o-tolyl)methanone (840 mg, 91%) was isolated as a colorless solid; LCMS: m/z (M+H)⁺=316.0.

### Scheme 13, Step 3

A mixture of (3-(2-(benzo[d][1,3]dioxol-5-yl)acetamido)phenyl)boronic acid (77 mg, 0.257 mmol), (5-bromoindolin-1-yl)(o-tolyl)methanone (63 mg, 0.198 mmol), tetrakis(triphenylphosphine)palladium(0) (23 mg, 0.020 mmol), and sodium carbonate (300 μL of a 2M aqueous solution, 0.594 mmol) in DME (1 ml) was heated with stirring at 100° C. overnight. The reaction mixture was concentrated under a stream of air. The residue was taken up in EtOAc, dried with MgSO₄, and filtered through an Agilent PL-Thiol MP SPE cartridge to remove palladium. The organic layer was concentrated under a stream of air. The residue was taken up in DMSO and subsequently purified by reverse phase chromatography to give Compound 31.

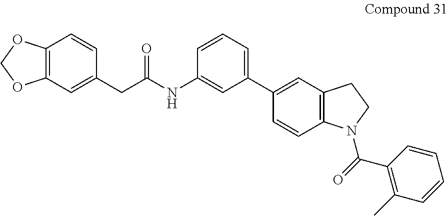 [SMILES: Cc1ccccc1C(=O)N1CCc2cc(-c3cccc(NC(=O)Cc4ccc5c(c4)OCO5)c3)ccc21]

HRMS: m/z (M+H)⁺=491.1957 (Calculated for C₂₈H₂₆N₃O₅S₂=491.1965); ¹H NMR analysis at room temperature is complicated by amide rotamers that are present as a result of the ortho-methyl group in the indolin-1-yl(o-tolyl)methanone segment of the molecule.

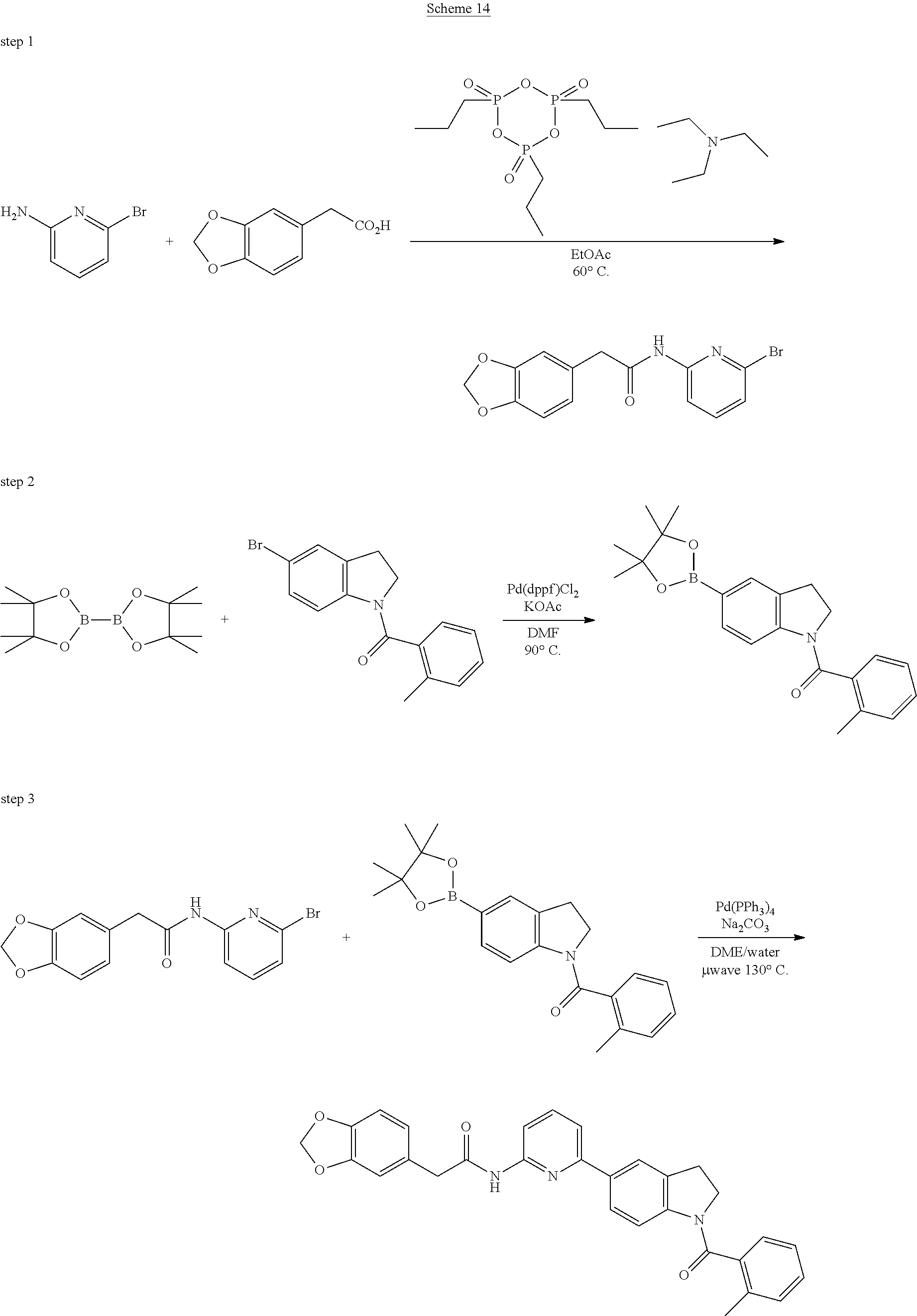

Scheme 14, Step 1

To a solution of 6-bromopyridin-2-amine (200 mg, 1.16 mmol), 2-(benzo[d][1,3]dioxol-5-yl)acetic acid (230 mg, 1.27 mmol), and triethylamine (0.8 mL, 5.8 mmol) in EtOAc (20 ml) was added 50 wt. % propylphosphonic anhydride solution in EtOAc (1.2 mL, 2.3 mmol). The mixture was stirred at rt for 5 min and then heated at 60° C. for 5 hr. The reaction mixture was cooled to rt and diluted with water, 0.5N NaOH, and EtOAc. The layers were separated and the aqueous layer was reextracted with EtOAc. The combined organic layers were dried with MgSO₄ and concentrated in vacuo to afford 2-(benzo[d][1,3]dioxol-5-yl)-N-(6-bromopyridin-2-yl)acetamide; LCMS: m/z (M+H)⁺=335.0. This material was used without purification in step 3 of Scheme 15.

### Scheme 14, Step 2

A mixture of (5-bromoindolin-1-yl)(o-tolyl)methanone (100 mg, 0.316 mmol), bis(pinacolato)diboron (120 mg, 0.474 mmol), [1,1′-bis(diphenylphosphino)ferrocene]dichloropalladium(II) (12 mg, 0.016 mmol), and potassium acetate (93 mg, 0.949 mmol) in DMF (3 ml) was heated with stirring at 80° C. 3 hr and at 90° C. for 2 hr. The reaction mixture was cooled to rt and diluted with water and EtOAc. The layers were separated and the aqueous layer was reextracted with EtOAc. The combined organic layers were dried with MgSO₄, concentrated in vacuo, and purified by silica gel chromatography (gradient 0 to 40% EtOAc/hexanes) to afford (5-(4,4,5,5-tetramethyl-1,3,2-dioxaborolan-2-yl)indolin-1-yl)(o-tolyl)methanone (71 mg, 62%) as a colorless foam; LCMS: m/z (M+H)⁺=364.2.

### Scheme 14, Step 3

A mixture of 2-(benzo[d][1,3]dioxol-5-yl)-N-(6-bromopyridin-2-yl)acetamide (47 mg 0.139 mmol), (5-(4,4,5,5-tetramethyl-1,3,2-dioxaborolan-2-yl)indolin-1-yl)(o-tolyl)methanone (36 mg, 0.10 mmol), tetrakis(triphenylphosphine)palladium(0) (12 mg, 0.010 mmol), and sodium carbonate (150 μL of a 2M aqueous solution, 0.297 mmol) in DME (1 ml) was heated in a microwave with stirring at 130° C. for 1 hr. The reaction mixture was concentrated under a stream of air. The residue was taken up in EtOAc, dried with MgSO₄, and filtered through an Agilent PL-Thiol MP SPE cartridge to remove palladium. The organic layer was concentrated under a stream of air. The residue was taken up in DMSO and subsequently purified by reverse phase chromatography to afford Compound 28.

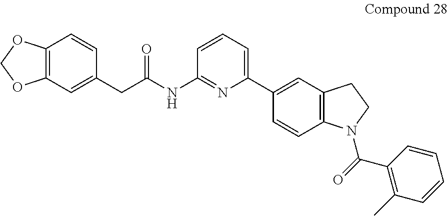 [SMILES: Cc1ccccc1C(=O)N1CCc2cc(-c3cccc(NC(=O)Cc4ccc5c(c4)OCO5)n3)ccc21]

HRMS: m/z (M+H)⁺=492.1922 (Calculated for C₃₀H₂₆N₃O₄=492.1918); ¹H NMR analysis at room temperature is complicated by amide rotamers that are present as a result of the ortho-methyl group in the indolin-1-yl(o-tolyl)methanone segment of the molecule.

COMPOUND 66 was prepared according to the method described in Scheme 7, step 4 substituting 2-(piperidin-1-yl)acetic acid for 2-(benzo[d][1,3]dioxol-5-yl)acetic acid.

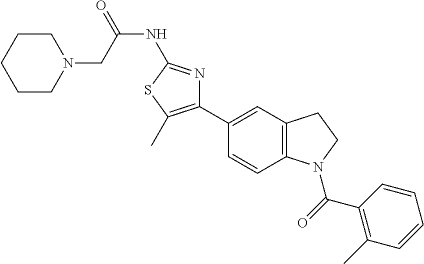 [SMILES: Cc1ccccc1C(=O)N1CCc2cc(-c3nc(NC(=O)CN4CCCCC4)sc3C)ccc21]

HRMS: m/z (M+H)⁺=475.2166 (Calculated for C₂₇H₃₁N₄O₂S=475.2162); ¹H NMR analysis at room temperature is complicated by amide rotamers that are present as a result of the ortho-methyl group in the indolin-1-yl(o-tolyl)methanone segment of the molecule.

COMPOUND 60 was prepared according to the method described in Scheme 7, step 4 substituting 2-(1-(tert-butoxycarbonyl)piperidin-4-yl)acetic acid for 2-(benzo[d][1,3]dioxol-5-yl)acetic acid.

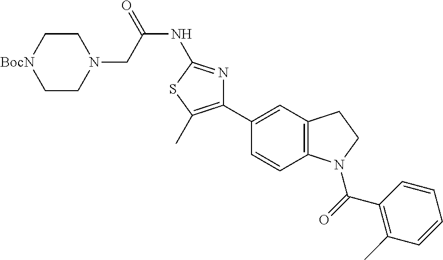 [SMILES: Cc1ccccc1C(=O)N1CCc2cc(-c3nc(NC(=O)CN4CCN(C(=O)OC(C)(C)C)CC4)sc3C)ccc21]

HRMS: m/z (M+H)⁺=575.2685 (Calculated for C₃₂H₃₉N₄O₄S=575.2687); ¹H NMR analysis at room temperature is complicated by amide rotamers that are present as a result of the ortho-methyl group in the indolin-1-yl(o-tolyl)methanone segment of the molecule.

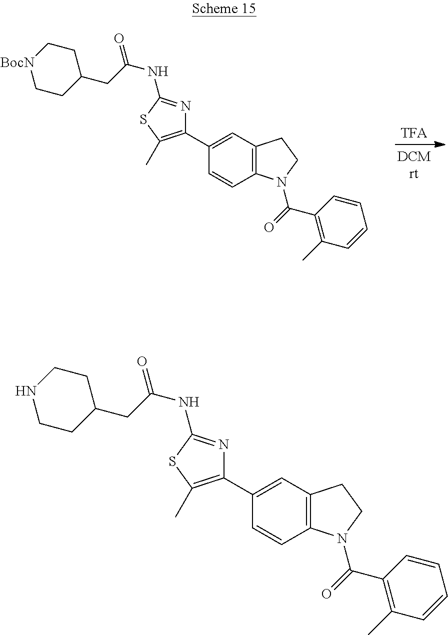 [SMILES: Cc1ccccc1C(=O)N1CCc2cc(-c3nc(NC(=O)CC4CCN(C(=O)OC(C)(C)C)CC4)sc3C)ccc21 ; Cc1ccccc1C(=O)N1CCc2cc(-c3nc(NC(=O)CC4CCNCC4)sc3C)ccc21 ; "TFA"]

### Scheme 15, Compound 73

To a solution of tert-butyl 4-(2-((5-methyl-4-(1-(2-methylbenzoyl)indolin-5-yl)thiazol-2-yl)amino)-2-oxoethyl)piperidine-1-carboxylate (COMPOUND 60) (60 mg, 0.104 mmol) in DCM (2 ml) was added trifluoroacetic acid (100 μL, 1.3 mmol). The reaction mixture stirred at rt 1 hr. The reaction mixture was concentrated under a stream of air. The residue was taken up in DMSO and subsequently purified by reverse phase chromatography to give COMPOUND 73.

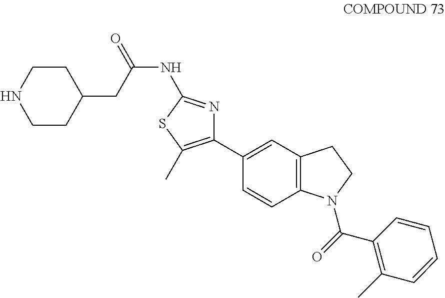 [SMILES: Cc1ccccc1C(=O)N1CCc2cc(-c3nc(NC(=O)CC4CCNCC4)sc3C)ccc21]

HRMS: m/z (M+H)⁺=475.2163 (Calculated for C₂₇H₃₁N₄O₂S=475.2162); ¹H NMR analysis at room temperature is complicated by amide rotamers that are present as a result of the ortho-methyl group in the indolin-1-yl(o-tolyl)methanone segment of the molecule.

COMPOUND 78 was prepared according to the method described in Scheme 14, step 3 substituting 4-(4-(4,4,5,5-tetramethyl-1,3,2-dioxaborolan-2-yl)phenyl)morpholine for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

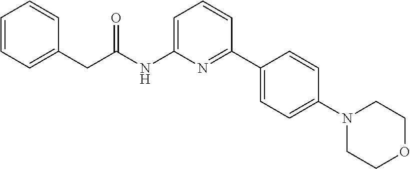 [SMILES: O=C(Cc1ccccc1)Nc1cccc(-c2ccc(N3CCOCC3)cc2)n1]

¹H NMR (400 MHz, DMSO-d₆) δ 10.74 (s, 1H), 10.58 (s, 1H), 8.13-7.85 (m, 3H), 7.74 (t, J=7.9 Hz, 1H), 7.53 (dd, J=7.7, 0.8 Hz, 1H), 7.39-7.18 (m, 5H), 7.06-6.97 (m, 2H), 3.79-3.70 (m, 6H), 3.22-3.15 (m, 4H); HRMS: m/z (M+H)⁺=374.1867 (Calculated for C₂₃H₂₄N₃O₂=374.1863).

COMPOUND 57 was prepared according to the method described in Scheme 14, step 3 substituting 5-(4,4,5,5-tetramethyl-1,3,2-dioxaborolan-2-yl)-1H-benzo[d]imidazole for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

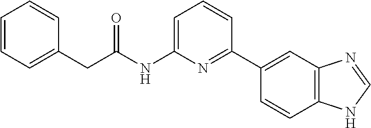 [SMILES: O=C(Cc1ccccc1)Nc1cccc(-c2ccc3[nH]cnc3c2)n1]

¹H NMR (400 MHz, DMSO-d₆) δ 10.75 (s, 1H), 9.19-9.14 (m, 1H), 8.40 (dd, J=1.6, 0.7 Hz, 1H), 8.18 (dd, J=8.7, 1.6 Hz, 1H), 8.03 (d, J=8.2 Hz, 1H), 7.91-7.78 (m, 2H), 7.74 (dd, J=7.7, 0.9 Hz, 1H), 7.40-7.19 (m, 5H), 3.77 (s, 2H); HRMS: m/z (M+H)⁺=329.1395 (Calculated for C₂₀H₁₇N₄O=329.1397).

COMPOUND 79 was prepared according to the method described in Scheme 8 substituting 2-cyclohexylacetyl choride for acetyl chloride.

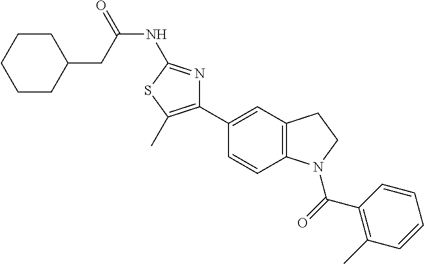 [SMILES: Cc1ccccc1C(=O)N1CCc2cc(-c3nc(NC(=O)CC4CCCCC4)sc3C)ccc21]

HRMS: m/z (M+H)⁺=474.2210 (Calculated for C₂₈H₃₂N₃O₂S=474.2210); ¹H NMR analysis at room temperature is complicated by amide rotamers that are present as a result of the ortho-methyl group in the indolin-1-yl(o-tolyl)methanone segment of the molecule.

COMPOUND 77 was prepared according to the method described in Scheme 7, step 4 substituting 2-morpholinoacetic acid for 2-(benzo[d][1,3]dioxol-5-yl)acetic acid.

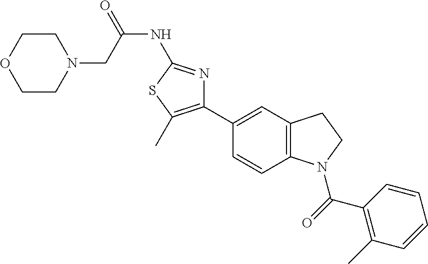 [SMILES: Cc1ccccc1C(=O)N1CCc2cc(-c3nc(NC(=O)CN4CCOCC4)sc3C)ccc21]

HRMS: m/z (M+H)⁺=477.1964 (Calculated for C₂₆H₂₉N₄O₃S=477.1955); ¹H NMR analysis at room temperature is complicated by amide rotamers that are present as a result of the ortho-methyl group in the indolin-1-yl(o-tolyl)methanone segment of the molecule.

COMPOUND 72 was prepared according to the method described in Scheme 7, step 4 substituting 2-(4-methylpiperazin-1-yl)acetic acid for 2-(benzo[d][1,3]dioxol-5-yl)acetic acid.

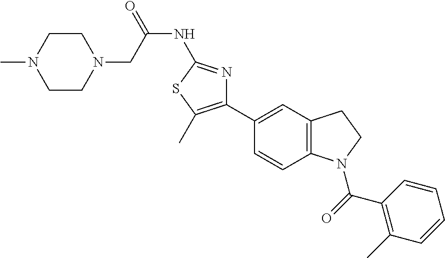 [SMILES: Cc1ccccc1C(=O)N1CCc2cc(-c3nc(NC(=O)CN4CCN(C)CC4)sc3C)ccc21]

HRMS: m/z (M+H)⁺=490.2271 (Calculated for C₂₇H₃₂N₅O₂S=490.2271); ¹H NMR analysis at room temperature is complicated by amide rotamers that are present as a result of the ortho-methyl group in the indolin-1-yl(o-tolyl)methanone segment of the molecule.

COMPOUND 75 was prepared according to the method described in Scheme 7, step 4 substituting 2-(1-methylpiperidin-4-yl)acetic acid hydrochloride for 2-(benzo[d][1,3]dioxol-5-yl)acetic acid and using additional triethylamine.

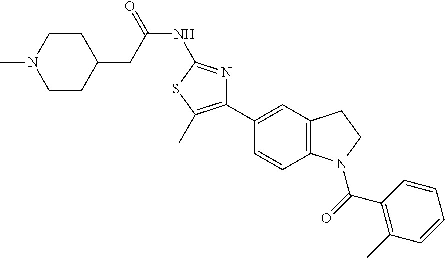 [SMILES: Cc1ccccc1C(=O)N1CCc2cc(-c3nc(NC(=O)CC4CCN(C)CC4)sc3C)ccc21]

HRMS: m/z (M+H)⁺=489.2322 (Calculated for C₂₈H₃₃N₄O₂S=489.2319); ¹H NMR analysis at room temperature is complicated by amide rotamers that are present as a result of the ortho-methyl group in the indolin-1-yl(o-tolyl)methanone segment of the molecule.

COMPOUND 69 was prepared according to the method described in Scheme 7, step 4 substituting 2-(4-(tert-butoxycarbonyl)piperazin-1-yl)acetic acid for 2-(benzo[d][1,3]dioxol-5-yl)acetic acid.

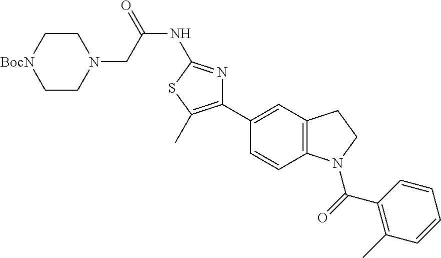 [SMILES: Cc1ccccc1C(=O)N1CCc2cc(-c3nc(NC(=O)CN4CCN(C(=O)OC(C)(C)C)CC4)sc3C)ccc21]

HRMS: m/z (M+H)⁺=576.2626 (Calculated for C₃₁H₃₈N₅O₄S=576.2639); ¹H NMR analysis at room temperature is complicated by amide rotamers that are present as a result of the ortho-methyl group in the indolin-1-yl(o-tolyl)methanone segment of the molecule.

COMPOUND 70 was prepared according to the method described in Scheme 15 substituting tert-butyl 4-(2-((5-methyl-4-(1-(2-methylbenzoyl)indolin-5-yl)thiazol-2-yl)amino)-2-oxoethyl)piperazine-1-carboxylate for tert-butyl 4-(2-((5-methyl-4-(1-(2-methylbenzoyl)indolin-5-yl)thiazol-2-yl)amino)-2-oxoethyl)piperidine-1-carboxylate (COMPOUND 60).

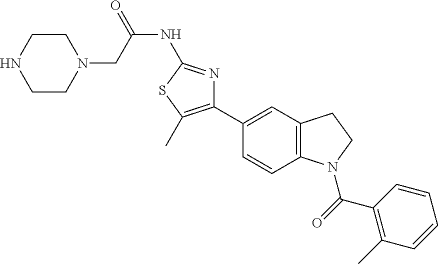 [SMILES: Cc1ccccc1C(=O)N1CCc2cc(-c3nc(NC(=O)CN4CCNCC4)sc3C)ccc21]

HRMS: m/z (M+H)⁺=476.2107 (Calculated for C₂₆H₃₀N₅O₂S=476.2115); ¹H NMR analysis at room temperature is complicated by amide rotamers that are present as a result of the ortho-methyl group in the indolin-1-yl(o-tolyl)methanone segment of the molecule.

COMPOUND 84 was prepared according to the method described in Scheme 7, step 4 substituting 2-(tetrahydro-2H-pyran-4-yl)acetic acid for 2-(benzo[d][1,3]dioxol-5-yl)acetic acid.

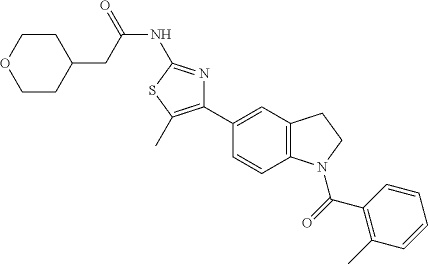 [SMILES: Cc1ccccc1C(=O)N1CCc2cc(-c3nc(NC(=O)CC4CCOCC4)sc3C)ccc21]

HRMS: m/z (M+H)⁺=476.1995 (Calculated for C₂₇H₃₀N₃O₃S=476.2002); ¹H NMR analysis at room temperature is complicated by amide rotamers that are present as a result of the ortho-methyl group in the indolin-1-yl(o-tolyl)methanone segment of the molecule.

COMPOUND 80 was prepared according to the method described in Scheme 14, step 3 substituting 4-(4,4,5,5-tetramethyl-1,3,2-dioxaborolan-2-yl)aniline for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

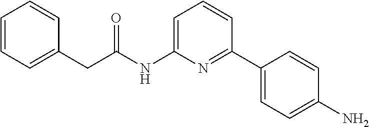 [SMILES: Nc1ccc(-c2cccc(NC(=O)Cc3ccccc3)n2)cc1]

¹H NMR (400 MHz, DMSO-d6) δ 10.53 (s, 1H), 7.83 (dd, J=8.3, 4.1 Hz, 3H), 7.71 (t, J=7.9 Hz, 1H), 7.46 (dd, J=7.7, 0.8 Hz, 1H), 7.38-7.18 (m, 5H), 6.73 (d, J=8.2 Hz, 2H), 3.74 (s, 2H); HRMS: m/z (M+H)⁺=304.1435 (Calculated for C₁₉H₁₈N30=304.1444).

COMPOUND 64 was prepared according to the method described in Scheme 14, step 3 substituting N-methyl-4-(4,4,5,5-tetramethyl-1,3,2-dioxaborolan-2-yl)aniline for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

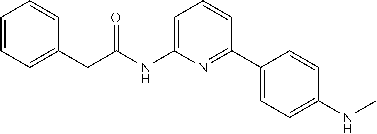 [SMILES: CNc1ccc(-c2cccc(NC(=O)Cc3ccccc3)n2)cc1]

¹H NMR (400 MHz, DMSO-d6) δ ppm 10.53 (s, 1H), 7.92-7.78 (m, 3H), 7.70 (t, J=7.9 Hz, 1H), 7.45 (dd, J=7.8, 0.8 Hz, 1H), 7.40-7.18 (m, 5H), 6.67-6.59 (m, 2H), 3.74 (s, 2H), 2.72 (s, 3H); HRMS: m/z (M+H)⁺=318.1598 (Calculated for C₂₀H₂₀N₃O=318.1601).

COMPOUND 63 was prepared according to the method described in Scheme 14, step 3 substituting (4-(piperidin-1-yl)phenyl)boronic acid hydrochloride for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

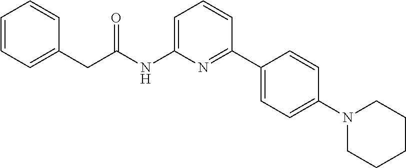 [SMILES: O=C(Cc1ccccc1)Nc1cccc(-c2ccc(N3CCCCC3)cc2)n1]

¹H NMR (400 MHz, DMSO-d6) δ 10.58 (s, 1H), 7.96 (d, J=8.4 Hz, 2H), 7.92-7.85 (m, 1H), 7.74 (t, J=7.9 Hz, 1H), 7.53 (d, J=7.7 Hz, 1H), 7.39-7.18 (m, 5H), 7.09 (s, 2H), 3.74 (s, 2H), 3.27 (d, J=6.0 Hz, 4H), 1.64 (s, 4H), 1.57 (d, J=6.6 Hz, 2H); HRMS: m/z (M+H)⁺=372.2080 (Calculated for C₂₄H₂₆N₃O=372.2070).

COMPOUND 88 was prepared according to the method described in Scheme 14, step 3 substituting 5-(4,4,5,5-tetramethyl-1,3,2-dioxaborolan-2-yl)-1H-pyrrolo[2,3-b]pyridine for (5-(4,4,5,5-tetramethyl-1,3,2-dioxaborolan-2-yl)indolin-1-yl)(o-tolyl)methanone, SiliaCat® DPP-Pd for tetrakis(triphenylphosphine)palladium(0), and heating in the microwave at 140° C. 40 min.

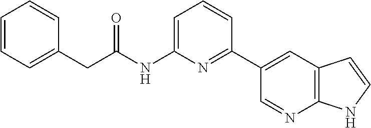 [SMILES: O=C(Cc1ccccc1)Nc1cccc(-c2cnc3[nH]ccc3c2)n1]

¹H NMR (400 MHz, DMSO-d6) δ 11.79 (d, J=2.6 Hz, 1H), 10.69 (s, 1H), 8.94 (d, J=2.1 Hz, 1H), 8.61 (dd, J=2.1, 0.7 Hz, 1H), 8.01-7.94 (m, 1H), 7.82 (t, J=7.9 Hz, 1H), 7.70 (dd, J=7.7, 0.8 Hz, 1H), 7.52 (dd, J=3.4, 2.5 Hz, 1H), 7.40-7.19 (m, 5H), 6.54 (dd, J=3.4, 1.8 Hz, 1H), 3.76 (s, 2H); HRMS: m/z (M+H)⁺=329.1388 (Calculated for C₂₀H₁₇N₄O=329.1397).

COMPOUND 76 was prepared according to the method described in Scheme 10, step 2 substituting N-(6-(1H-pyrrolo[2,3-b]pyridin-5-yl)pyridin-2-yl)-2-phenylacetamide for N-(4-(1H-indol-5-yl)-5-methylthiazol-2-yl)-2-(benzo[d][1,3]dioxol-5-yl)acetamide.

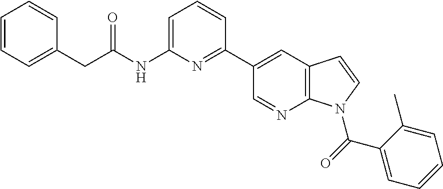 [SMILES: Cc1ccccc1C(=O)n1ccc2cc(-c3cccc(NC(=O)Cc4ccccc4)n3)cnc21]

¹H NMR (400 MHz, DMSO-d₆) δ 10.71 (s, 1H), 8.82 (dd, J=2.2, 0.4 Hz, 1H), 8.68 (d, J=2.2 Hz, 1H), 8.01 (dd, J=8.1, 0.8 Hz, 1H), 7.93 (d, J=4.1 Hz, 1H), 7.83 (t, J=7.9 Hz, 1H), 7.73 (dd, J=7.7, 0.8 Hz, 1H), 7.53-7.18 (m, 10H), 6.94 (d, J=4.1 Hz, 1H), 3.74 (s, 2H), 2.21 (s, 3H); HRMS: m/z (M+H)⁺=477.1816 (Calculated for C₂₈H₂₃N₄O₂=477.1816).

COMPOUND 65 was prepared according to the method described in Scheme 14, step 3 substituting 4-(6-bromopyridin-2-yl)morpholine for 2-(benzo[d][1,3]dioxol-5-yl)-N-(6-bromopyridin-2-yl)acetamide.

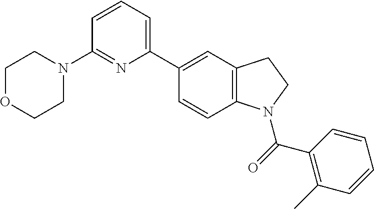 [SMILES: Cc1ccccc1C(=O)N1CCc2cc(-c3cccc(N4CCOCC4)n3)ccc21]

HRMS: m/z (M+H)⁺=400.2023 (Calculated for C₂₅H₂₆N₃O₂=400.2020); ¹H NMR analysis at room temperature is complicated by amide rotamers that are present as a result of the ortho-methyl group in the indolin-1-yl(o-tolyl)methanone segment of the molecule.

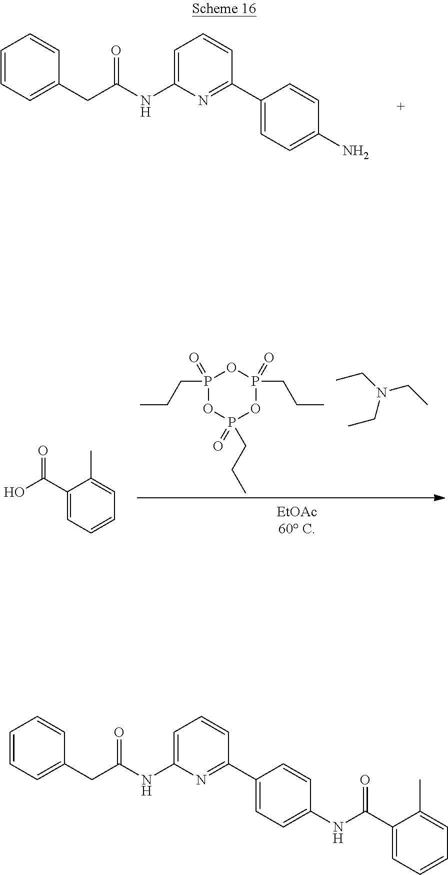

Scheme 16 To a solution of N-(6-(4-aminophenyl)pyridin-2-yl)-2-phenylacetamide (COMPOUND 80) (55 mg, 0.181 mmol), 2-methylbenzoic acid (40 mg, 0.290 mmol), and triethylamine (130 μL, 5.8 mmol) in EtOAc (3 ml) was added 50 wt. % propylphosphonic anhydride solution in EtOAc (0.26 mL, 0.44 mmol). The mixture was stirred at rt for 5 min and then heated at 60° C. for 3 hr. The reaction mixture was cooled to rt and diluted with water and EtOAc. The layers were separated and the aqueous layer was reextracted with EtOAc. The combined organic layers were dried with MgSO₄ and concentrated in vacuo to afford a residue which was taken up in DMSO and subsequently purified by reverse phase chromatography to afford COMPOUND 71.

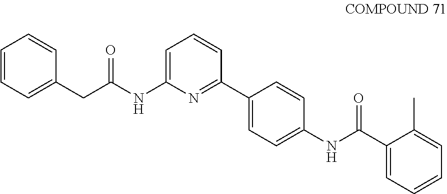 [SMILES: Cc1ccccc1C(=O)Nc1ccc(-c2cccc(NC(=O)Cc3ccccc3)n2)cc1]

¹H NMR (400 MHz, DMSO-d₆) δ 10.66 (s, 1H), 10.43 (s, 1H), 8.10-8.00 (m, 2H), 7.96 (d, J=8.2 Hz, 1H), 7.89-7.76 (m, 3H), 7.61 (dd, J=7.7, 0.8 Hz, 1H), 7.50-7.19 (m, 10H), 3.76 (s, 2H), 2.39 (s, 3H), ¹H NMR analysis at room temperature is complicated by amide rotamers that are present as a result of the ortho-methyl group in the 2-methyl-N-phenylbenzamide region of the molecule; HRMS: m/z (M+H)⁺=422.1869 (Calculated for C₂₇H₂₄N₃O₂=422.1863).

COMPOUND 68 was prepared according to the method described in Scheme 16 substituting N-(6-(4-(methylamino)phenyl)pyridin-2-yl)-2-phenylacetamide for N-(6-(4-aminophenyl)pyridin-2-yl)-2-phenylacetamide (COMPOUND 80).

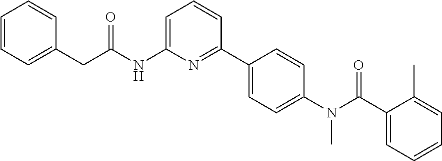 [SMILES: Cc1ccccc1C(=O)N(C)c1ccc(-c2cccc(NC(=O)Cc3ccccc3)n2)cc1]

¹H NMR (400 MHz, DMSO-d₆) δ 10.64 (s, 1H), 7.96 (d, J=8.2 Hz, 1H), 7.89 (s, 2H), 7.78 (t, J=7.9 Hz, 1H), 7.56 (d, J=7.7 Hz, 1H), 7.37-7.18 (m, 7H), 7.11 (d, J=8.6 Hz, 4H), 7.01 (s, 1H), 3.72 (s, 2H), 3.38 (s, 3H), 2.24 (s, 3H), ¹H NMR analysis at room temperature is complicated by amide rotamers that are present as a result of the ortho-methyl group in the N,2-dimethyl-N-phenylbenzamide region of the molecule; HRMS: m/z (M+H)⁺=436.2008 (Calculated for C₂₈H₂₆N₃O₂=436.2020).

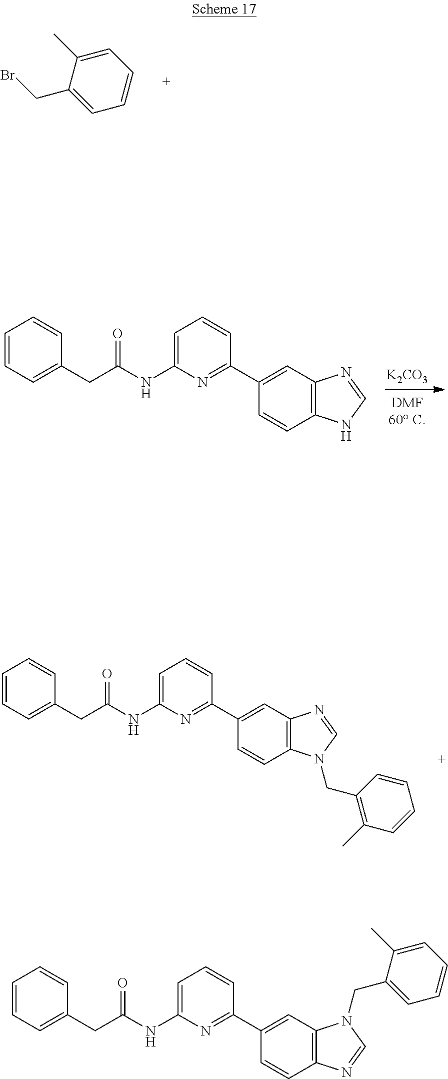

### Scheme 17

To a solution of N-(6-(1H-benzo[d]imidazol-5-yl)pyridin-2-yl)-2-phenylacetamide (COMPOUND 57) (60 mg, 0.183 mmol) and potassium carbonate (63 mg, 0.457 mmol) in DMF (2.5 ml) was added 1-(bromomethyl)-2-methylbenzene (0.27 μL, 0.20 mmol). The mixture was stirred at rt for 5 min and then heated at 60° C. for 4 hr. The reaction mixture was cooled to rt and diluted with water and EtOAc. The layers were separated and the aqueous layer was reextracted with EtOAc. The combined organic layers were dried with MgSO₄ and concentrated in vacuo to afford a residue which was taken up in DMSO and subsequently purified by reverse phase chromatography to afford COMPOUND 89 as a mixture of isomers.

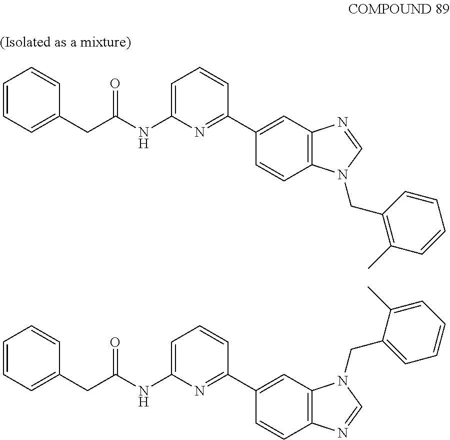

HRMS: m/z (M+H)⁺=433.2029 (Calculated for C₂₈H₂₅N₄O=433.2023).

COMPOUND 109 was prepared according to the method described in Scheme 8 substituting phenylmethanesulfonyl chloride for acetyl chloride.

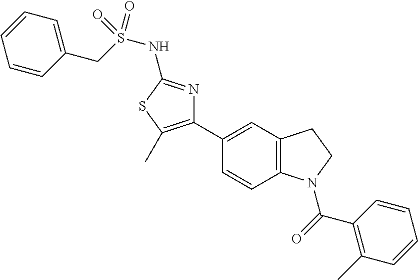 [SMILES: Cc1ccccc1C(=O)N1CCc2cc(-c3nc(NS(=O)(=O)Cc4ccccc4)sc3C)ccc21]

HRMS: m/z (M+H)⁺=526.1210 (Calculated for C₂₇H₂₅NaN₃O₃S₂=526.1230); ¹H NMR analysis at room temperature is complicated by amide rotamers that are present as a result of the ortho-methyl group in the indolin-1-yl(o-tolyl)methanone segment of the molecule.

COMPOUND 232 was prepared according to the method described in Scheme 7 substituting 2-methylindoline for indoline in step 1.

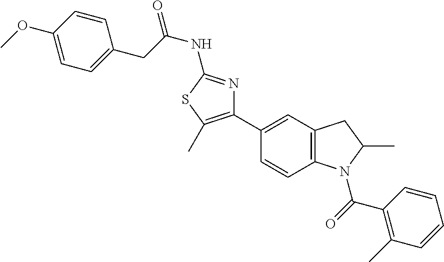 [SMILES: COc1ccc(CC(=O)Nc2nc(-c3ccc4c(c3)CC(C)N4C(=O)c3ccccc3C)c(C)s2)cc1]

HRMS: m/z (M+H)⁺=512.2016 (Calculated for C₃₀H₃₀N₃O₃S=512.2002).

COMPOUND 112 was prepared according to the method described in Scheme 7, step 4 substituting 2-(6-methoxypyridin-3-yl)acetic acid for 2-(benzo[d][1,3]dioxol-5-yl)acetic acid.

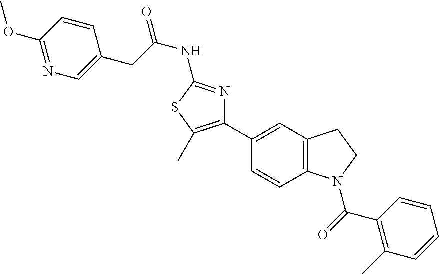 [SMILES: COc1ccc(CC(=O)Nc2nc(-c3ccc4c(c3)CCN4C(=O)c3ccccc3C)c(C)s2)cn1]

HRMS: m/z (M+H)⁺=499.1807 (Calculated for C₂₈H₂₇N₄O₃S=499.1798); ¹H NMR analysis at room temperature is complicated by amide rotamers that are present as a result of the ortho-methyl group in the indolin-1-yl(o-tolyl)methanone segment of the molecule.

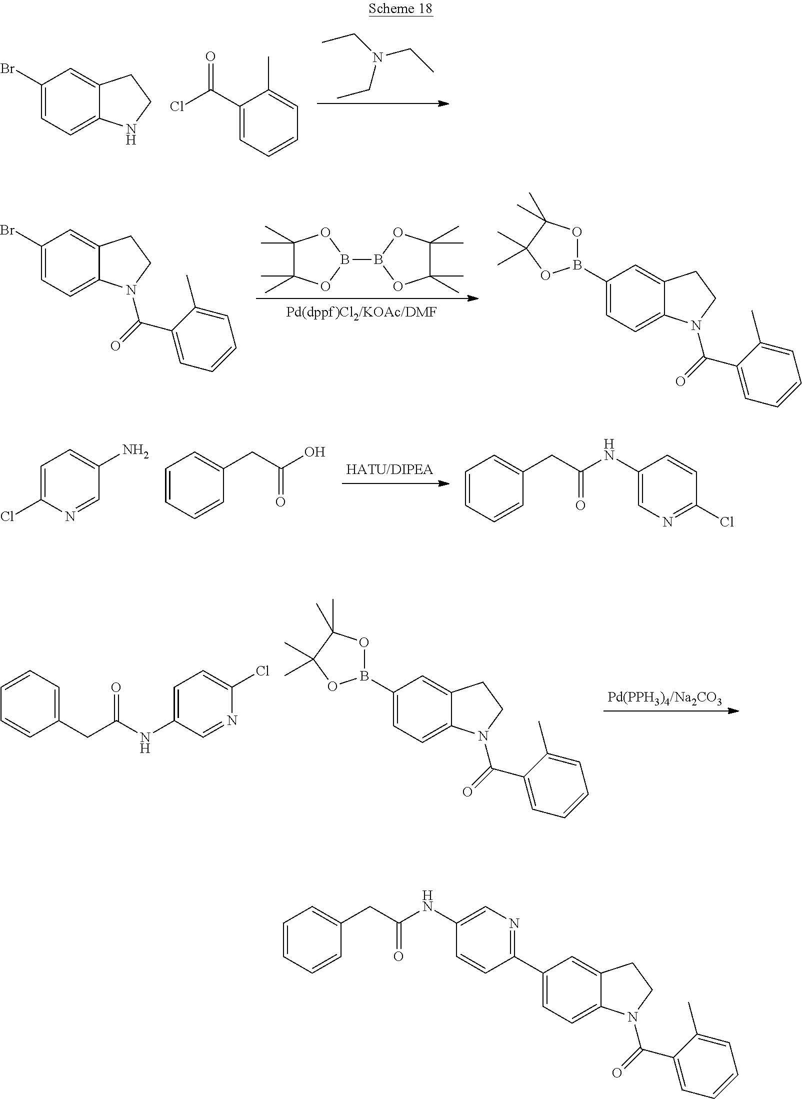

Scheme 18, step 1 COMPOUND 107. To a solution of 5-bromoindoline (3 g, 15.15 mmol) and Et₃N (4.22 ml, 30.3 mmol) in DCM (25 ml) was added dropwise 2-methylbenzoyl chloride (2.075 ml, 15.90 mmol) at ice bath. The reaction mixture was stirred at 0° C. for 5 hrs. The mixture was diluted with DCM/0.5N NaOH (40 ml). The organic layer was dried over MgSO₄ and concentrated. The crude product was purified by Biotage (0-30%, EtOAc/hex); LCMS: m/z (M+H)⁺=316.0/318.0. ¹H NMR (400 MHz, DMSO-d6) δ 8.17-8.07 (m, 1H), 7.38 (dd, J=53.7, 20.2 Hz, 6H), 3.72 (d, J=9.3 Hz, 2H), 3.09 (t, J=8.3 Hz, 2H), 2.26 (s, 3H).

### Scheme 18, Step 2.

The mixture of (5-bromoindolin-1-yl)(o-tolyl)methanone (0.5 g, 1.581 mmol), BIS(PINACOLATO)DIBORON (0.602 g, 2.372 mmol), PdCl₂(dppf) (0.058 g, 0.079 mmol) and POTASSIUM ACETATE (0.466 g, 4.74 mmol) in DMF (15 ml) was stirred at 90° C. for overnight. Water was added to the mixture, and extracted with EtOAc. The organic layer was dried over MgSO₄ and concentrated. The crude product was purified by Biotage (0-30%, EtOAc/hex); LCMS: m/z (M+H)⁺=364.1.

Scheme 18, step 3 COMPOUND 106 was prepared according to the method described in Scheme 1 substituting 6-chloropyridin-3-amine for 5-phenylthiazol-2-amine and substituting 2-phenylacetic acid for 5-(3-nitrophenyl)furan-2-carboxylic acid; LCMS: m/z (M+H)⁺=247.0. ¹H NMR (400 MHz, DMSO-d6) δ 10.52 (s, 1H), 8.60 (dd, J=2.8, 0.6 Hz, 1H), 8.08 (dd, J=8.7, 2.8 Hz, 1H), 7.46 (dd, J=8.6, 0.6 Hz, 1H), 7.39-7.30 (m, 4H), 7.30-7.21 (m, 1H), 3.68 (s, 2H).

Scheme 18, step 4 COMPOUND 101.

A mixture of N-(6-chloropyridin-3-yl)-2-phenylacetamide (47.5 mg, 0.193 mmol), (5-(4,4,5,5-tetramethyl-1,3,2-dioxaborolan-2-yl)indolin-1-yl)(o-tolyl)methanone (50 mg, 0.138 mmol), Pd(Ph₃P)₄ (15.91 mg, 0.014 mmol) and Na₂CO₃ (0.206 ml, 0.413 mmol) in DME (1.3 ml) was heated in a microwave with stirring at 130° C. for 1 hr. The reaction mixture was concentrated under a stream of air. The residue was taken up in EtOAc, dried with MgSO₄, and filtered through an Agilent PL-Thiol MP SPE cartridge to remove palladium. The organic layer was concentrated under a stream of air. The residue was taken up in DMSO and subsequently purified by reverse phase chromatography to afford COMPOUND 101.

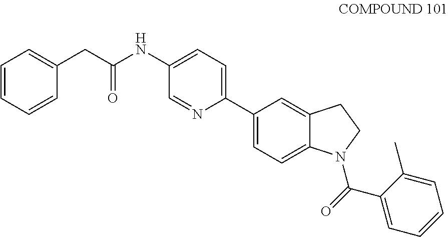 [SMILES: Cc1ccccc1C(=O)N1CCc2cc(-c3ccc(NC(=O)Cc4ccccc4)cn3)ccc21]

¹H NMR (400 MHz, DMSO-d6) δ 10.48 (s, 1H), 8.80 (s, 1H), 8.24 (d, J=8.3 Hz, 1H), 8.13 (d, J=8.7 Hz, 1H), 7.94 (d, J=13.8 Hz, 3H), 7.45-7.20 (m, 9H), 3.75 (s, 2H), 3.70 (s, 3H), 3.16 (d, J=9.1 Hz, 2H), 2.29 (s, 2H); Method 1, retention time: 5.022 min; HRMS: m/z (M+H)⁺=448.2004 (Calculated for C₂₉H₂₆N₃O₂=448.2020).

COMPOUND 102 was prepared according to the method described in Scheme 18, step 4 substituting N-(4-chloropyrimidin-2-yl)-2-phenylacetamide for N-(6-chloropyridin-3-yl)-2-phenylacetamide.

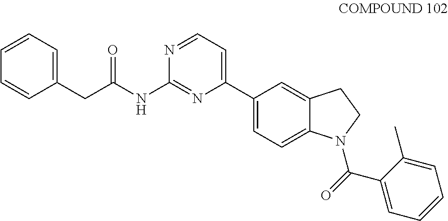 [SMILES: Cc1ccccc1C(=O)N1CCc2cc(-c3ccnc(NC(=O)Cc4ccccc4)n3)ccc21]

¹H NMR (400 MHz, DMSO-d₆) δ 10.79 (s, 1H), 8.65 (s, 1H), 8.28 (s, 1H), 8.10 (d, J=16.7 Hz, 1H), 7.77-7.48 (m, 2H), 7.47-7.16 (m, 9H), 3.81 (d, J=31.1 Hz, 4H), 3.17 (s, 2H), 2.28 (s, 3H); Method 1, retention time: 5.527 min; HRMS: m/z (M+H)⁺=449.1972 (Calculated for C₂₈H₂₅N₄O₂=449.1972).

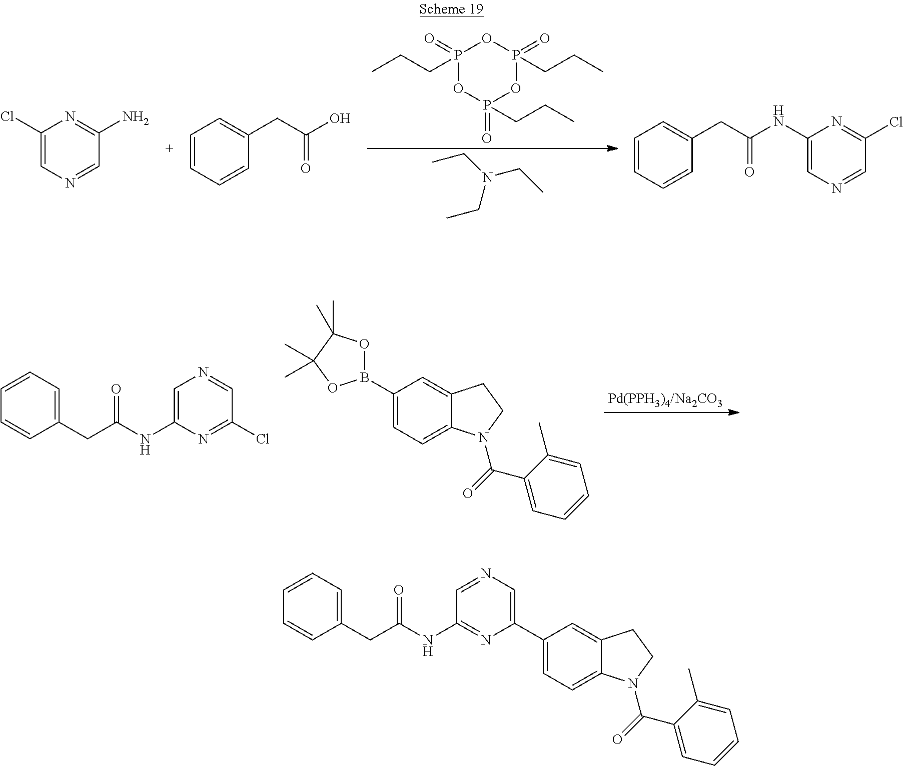

### Scheme 19, Step 1. Compound 105.

The mixture of 2-phenylacetic acid (0.578 g, 4.25 mmol), 6-chloropyrazin-2-amine (0.5 g, 3.86 mmol), 2,4,6-tripropyl-1,3,5,2,4,6-trioxatriphosphinane 2,4,6-trioxide (3.94 ml, 7.72 mmol)(i50% in EtOAc) and TRIETHYLAMINE (2.69 ml, 19.30 mmol) in EtOAc (10 ml) was stirred at 60° C. for 1 h. The solvent was removed. The crude product was purified by Biotage (0-50%, EtOAc/hex) to give white solid. LCMS: m/z (M+H)⁺=248.0577 (Calculated for C₁₂H₁₁ClN₃O=248.0585); ¹H NMR (400 MHz, DMSO-d6) δ 11.34 (s, 1H), 9.27 (d, J=0.6 Hz, 1H), 8.48 (d, J=0.6 Hz, 1H), 7.36-7.30 (m, 4H), 7.29-7.22 (m, 1H), 3.76 (s, 2H).

### Scheme 19, Step 2.

COMPOUND 104 was prepared according to the method described in Scheme 18, step 4 substituting N-(6-chloropyrazin-2-yl)-2-phenylacetamide for N-(6-chloropyridin-3-yl)-2-phenylacetamide.

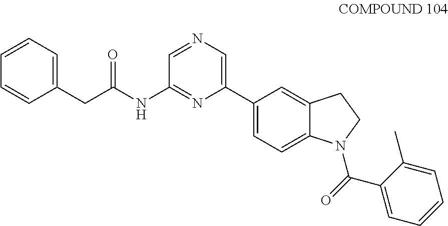 [SMILES: Cc1ccccc1C(=O)N1CCc2cc(-c3cncc(NC(=O)Cc4ccccc4)n3)ccc21]

¹H NMR (400 MHz, DMSO-d6) δ 11.05 (s, 1H), 9.21 (s, 1H), 8.93 (s, 1H), 8.06 (s, 2H), 7.47-7.29 (m, 10H), 3.78 (d, J=17.1 Hz, 4H), 3.18 (d, J=7.9 Hz, 2H), 2.30 (s, 3H); Method 1, retention time: 6.139 min; HRMS: m/z (M+H)⁺=449.1978 (Calculated for C₂₈H₂₅N₄O₂=449.1972).

COMPOUND 91 was prepared according to the method described in Scheme 18, step 4 substituting N-(6-bromopyridin-2-yl)-2-phenylacetamide for N-(6-chloropyridin-3-yl)-2-phenylacetamide and (1H-indazol-5-yl)boronic acid for (5-(4,4,5,5-tetramethyl-1,3,2-dioxaborolan-2-yl)indolin-1-yl)(o-tolyl)methanone.

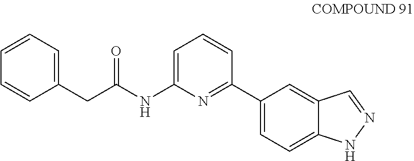 [SMILES: O=C(Cc1ccccc1)Nc1cccc(-c2ccc3[nH]ncc3c2)n1]

¹H NMR (400 MHz, DMSO-d6) δ 13.18 (s, 1H), 10.71 (s, 1H), 8.49 (dd, J=1.6, 0.8 Hz, 1H), 8.18 (d, J=1.0 Hz, 1H), 8.12 (dd, J=8.8, 1.6 Hz, 1H), 8.00-7.96 (m, 1H), 7.83 (t, J=7.9 Hz, 1H), 7.69 (dd, J=7.8, 0.8 Hz, 1H), 7.63 (dt, J=8.9, 1.0 Hz, 1H), 7.40-7.30 (m, 4H), 7.28-7.22 (m, 1H), 3.78 (s, 2H); Method 1, retention time: 4.970 min; HRMS: m/z (M+H)⁺=329.1382 (Calculated for C₂₀H₁₇N₄O=329.1397).

COMPOUND 92 was prepared according to the method described in Scheme 18, step 4 substituting N-(6-bromopyridin-2-yl)-2-phenylacetamide for N-(6-chloropyridin-3-yl)-2-phenylacetamide and 2-phenyl-5-(4,4,5,5-tetramethyl-1,3,2-dioxaborolan-2-yl)-1H-benzo[d]imidazole for (5-(4,4,5,5-tetramethyl-1,3,2-dioxaborolan-2-yl)indolin-1-yl)(o-tolyl)methanone.

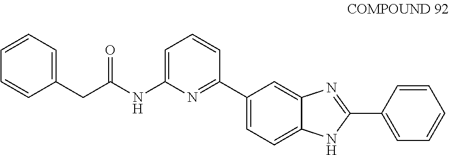 [SMILES: O=C(Cc1ccccc1)Nc1cccc(-c2ccc3[nH]c(-c4ccccc4)nc3c2)n1]

¹H NMR (400 MHz, DMSO-d6) δ 10.72 (s, 1H), 8.35-8.31 (m, 1H), 8.24-8.18 (m, 2H), 8.09-8.03 (m, 1H), 8.01 (d, J=8.2 Hz, 1H), 7.86 (t, J=7.9 Hz, 1H), 7.78-7.71 (m, 2H), 7.68-7.54 (m, 4H), 7.43-7.31 (m, 4H), 7.30-7.21 (m, 1H), 3.80 (s, 2H); Method 1, retention time: 4.586 min; HRMS: m/z (M+H)⁺=405.1696 (Calculated for C₂₆H₂₁N₄O=405.1710).

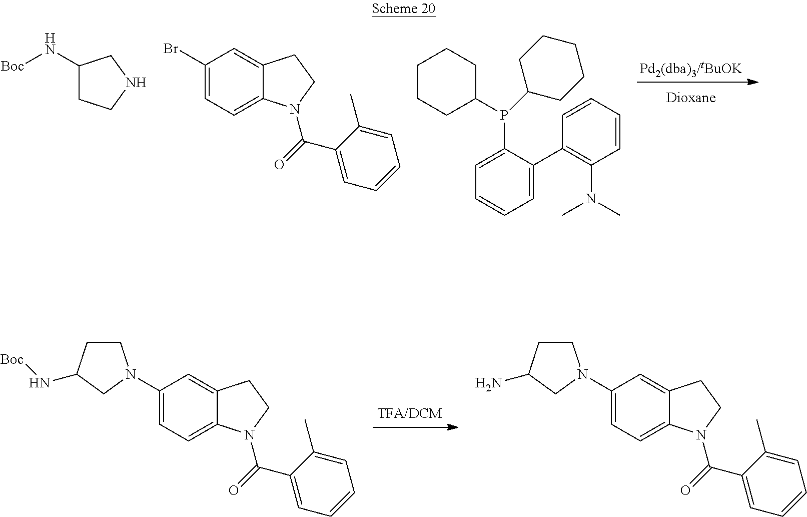

### Scheme 20, Step 1.

To a solution of (5-bromoindolin-1-yl)(o-tolyl)methanone (0.255 g, 0.805 mmol), 2′-(dicyclohexylphosphino)-N,N-dimethyl-[1,1′-biphenyl]-2-amine (0.042 g, 0.107 mmol) and Pd2(dba)3 (0.049 g, 0.054 mmol) in dioxane (3 ml) were added tert-butyl pyrrolidin-3-ylcarbamate (0.1 g, 0.537 mmol), and then POTASSIUM TERT-BUTOXIDE (0.120 g, 1.074 mmol) at r.t. The mixture was stirred at 90° C. for 3 h. After cooling, the solids were filtered off and the filtrate was concentrated. The crude product was purified by Biotage (0-50%, EtOAc/hex).

### Scheme 20, Step 2. Compound 93.

To a solution of tert-butyl (1-(1-(2-methylbenzoyl)indolin-5-yl)pyrrolidin-3-yl)carbamate (90 mg, 0.214 mmol) in DCM (5 ml) was added TFA (0.5 ml, 6.49 mmol). The reaction mixture was stirred at r.t. for 3 hrs. The solvent was removed. The crude product was purified by reverse phase chromatography to afford COMPOUND 93.

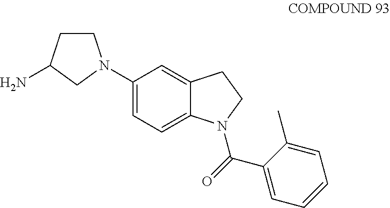 [SMILES: Cc1ccccc1C(=O)N1CCc2cc(N3CCC(N)C3)ccc21]

Method 1, retention time: 3.773 min; HRMS: m/z (M+H)⁺=322.1922 (Calculated for C₂₀H₂₄N₃O=322.1914);

COMPOUND 94 was prepared according to the method described in Scheme 20, step 1 substituting tert-butyl piperidin-3-ylcarbamate for tert-butyl pyrrolidin-3-ylcarbamate.

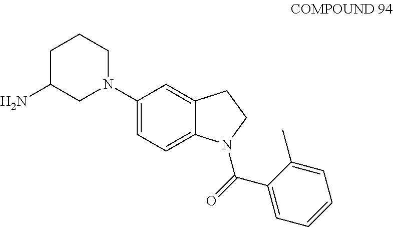 [SMILES: Cc1ccccc1C(=O)N1CCc2cc(N3CCCC(N)C3)ccc21]

Method 1, retention time: 3.836 min; HRMS: m/z (M+H)⁺=336.2066 (Calculated for C₂₁H₂₆N₃O=336.2070);

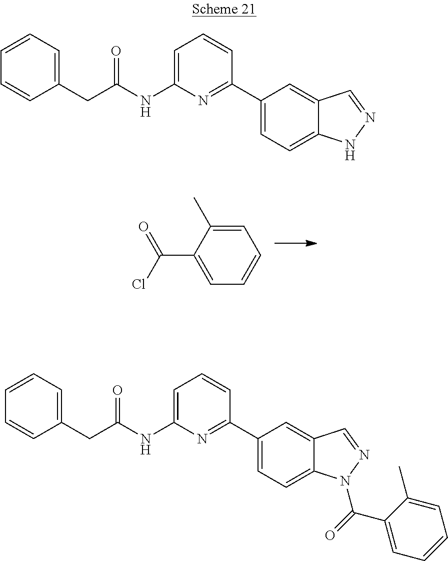

The mixture of N-(6-(1H-indazol-5-yl)pyridin-2-yl)-2-phenylacetamide (30 mg, 0.091 mmol) and 2-methylbenzoyl chloride (0.024 ml, 0.183 mmol) in THF (0.6 ml) was heated in a microwave with stirring at 120° C. for 20 min. The solid was filtered and washed with DCM. The filtrate was concentrated. The residue was taken up in DMSO and subsequently purified by reverse phase chromatography to afford COMPOUND 95.

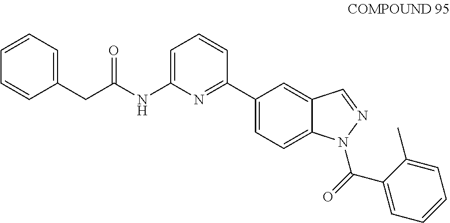 [SMILES: Cc1ccccc1C(=O)n1ncc2cc(-c3cccc(NC(=O)Cc4ccccc4)n3)ccc21]

¹H NMR (400 MHz, DMSO-d6) δ 10.81 (s, 1H), 8.63 (dd, J=1.7, 0.8 Hz, 1H), 8.58-8.51 (m, 2H), 8.47 (dd, J=8.8, 1.7 Hz, 1H), 8.07 (dd, J=8.3, 0.7 Hz, 1H), 7.91 (t, J=7.9 Hz, 1H), 7.79 (dd, J=7.7, 0.8 Hz, 1H), 7.55 (dd, J=7.6, 1.4 Hz, 1H), 7.48 (td, J=7.5, 1.4 Hz, 1H), 7.41-7.31 (m, 6H), 7.29-7.22 (m, 1H), 3.79 (s, 2H), 2.27 (s, 3H); Method 1, retention time: 6.787 min; HRMS: m/z (M+H)⁺=447.1798 (Calculated for C₂₈H₂₃N₄O₂=447.1816).

COMPOUND 96 was prepared according to the method described in Scheme 4 substituting (5-(3-aminopyrrolidin-1-yl)indolin-1-yl)(o-tolyl)methanone for 5-phenylthiazol-2-amine and substituting 2-phenylacetic acid for 5-(3-nitrophenyl)furan-2-carboxylic acid.

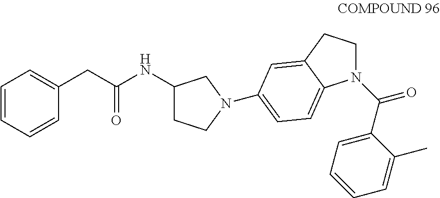 [SMILES: Cc1ccccc1C(=O)N1CCc2cc(N3CCC(NC(=O)Cc4ccccc4)C3)ccc21]

Method 1, retention time: 5.626 min; HRMS: m/z (M+H)⁺=440.2341 (Calculated for C₂₈H₃₀N₃O₂=440.2333); ¹H NMR analysis at room temperature is complicated by amide rotamers that are present as a result of the ortho-methyl group in the indolin-1-yl(o-tolyl)methanone segment of the molecule.

COMPOUND 97 was prepared according to the method described in Scheme 4 substituting (5-(3-aminopiperidin-1-yl)indolin-1-yl)(o-tolyl)methanone for 5-phenylthiazol-2-amine and substituting 2-phenylacetic acid for 5-(3-nitrophenyl)furan-2-carboxylic acid.

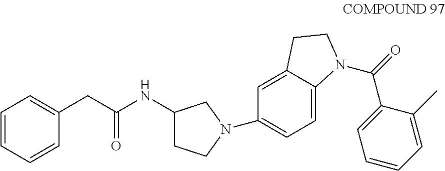 [SMILES: Cc1ccccc1C(=O)N1CCc2cc(N3CCC(NC(=O)Cc4ccccc4)C3)ccc21]

Method 1, retention time: 5.626 min; HRMS: m/z (M+H)⁺=454.2486 (Calculated for C₂₉H₃₂N₃O₂=454.2489); ¹H NMR analysis at room temperature is complicated by amide rotamers that are present as a result of the ortho-methyl group in the indolin-1-yl(o-tolyl)methanone segment of the molecule.

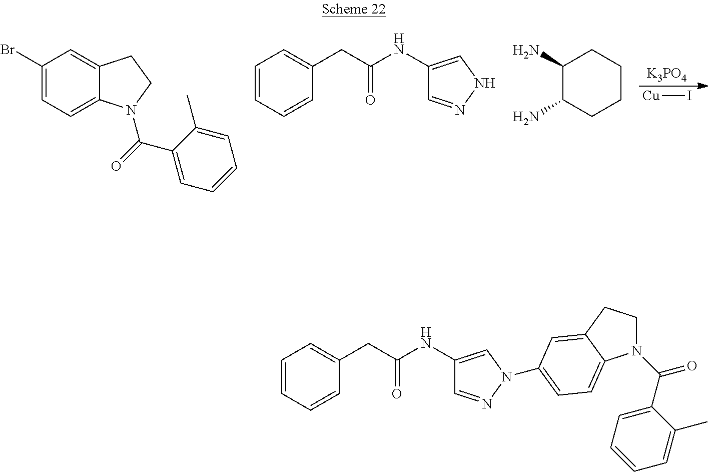

The mixture of (5-bromoindolin-1-yl)(o-tolyl)methanone (0.1 g, 0.316 mmol), 2-phenyl-N-(1H-pyrazol-4-yl)acetamide (76 mg, 0.38 mmol), TRANS-1,2-DIAMINOCYCLOHEXANE (3.80 μl, 0.032 mmol), K₃PO₄ (0.141 g, 0.664 mmol) and COPPER(I) IODIDE (3.01 mg, 0.016 mmol) in toluene (1 ml) was heated in a microwave with stirring at 120° C. for 1 hr. The reaction mixture was concentrated under a stream of air. The residue was taken up in EtOAc, dried with MgSO₄, and filtered through an Agilent PL-Thiol MP SPE cartridge to remove copper. The organic layer was concentrated under a stream of air. The residue was taken up in DMSO and subsequently purified by reverse phase chromatography to afford COMPOUND 100.

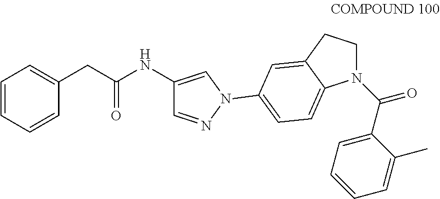 [SMILES: Cc1ccccc1C(=O)N1CCc2cc(-n3cc(NC(=O)Cc4ccccc4)cn3)ccc21]

Method 1, retention time: 5.468 min; HRMS: m/z (M+H)⁺=437.1977 (Calculated for C₂₇H₂₅N₄O₂=437.1972).

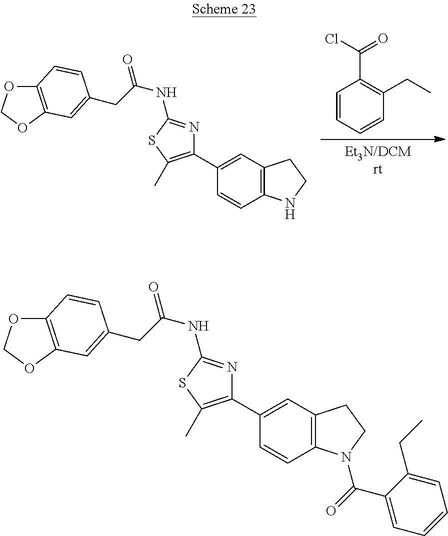 [SMILES: Cc1sc(NC(=O)Cc2ccc3c(c2)OCO3)nc1-c1ccc2c(c1)CCN2 ; CCc1ccccc1C(=O)Cl ; CCc1ccccc1C(=O)N1CCc2cc(-c3nc(NC(=O)Cc4ccc5c(c4)OCO5)sc3C)ccc21]

### Scheme 23 Compound 113.

To a solution of 2-(benzo[d][1,3]dioxol-5-yl)-N-(4-(indolin-5-yl)-5-methylthiazol-2-yl)acetamide (50 mg, 0.127 mmol) and triethylamine (35 μL, 0.254 mmol) in DCM (1.2 ml) was added 2-ethylbenzoyl chloride (21 μL, 0.140 mmol). The reaction stirred at rt for 2 hr. The reaction mixture was concentrated under a stream of air to afford a residue which was taken up in DMSO and subsequently purified by reverse phase chromatography to give COMPOUND 113.

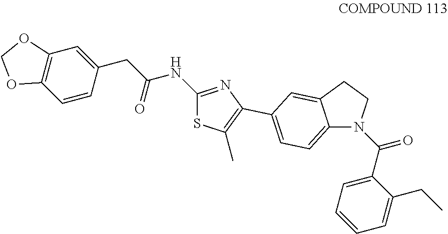 [SMILES: CCc1ccccc1C(=O)N1CCc2cc(-c3nc(NC(=O)Cc4ccc5c(c4)OCO5)sc3C)ccc21]

¹H NMR (400 MHz, DMSO-d6) δ ppm 12.26 (s, 1H), 8.22 (d, J=8.7 Hz, 1H), 7.51 (d, J=5.1 Hz, 2H), 7.42-7.28 (m, 4H), 6.91-6.81 (m, 2H), 6.81-6.74 (m, 1H), 5.97 (s, 2H), 3.72 (s, 2H), 3.63 (s, 2H), 3.16-3.04 (m, 2H), 2.61 (q, J=7.5 Hz, 2H), 2.44 (s, 3H), 1.16 (t, J=7.5 Hz, 3H); Method 1, retention time: 6.543 min; HRMS: m/z (M+H)⁺=526.1799 (Calculated for C₃₀H₂₈N₃O₄S=526.1795).

COMPOUND 114 was prepared according to the method described in Scheme 23 substituting 2-(trifluoromethoxy)benzoyl chloride for 2-ethylbenzoyl chloride.

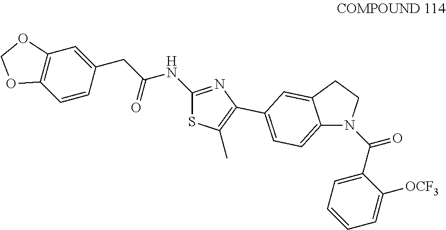 [SMILES: Cc1sc(NC(=O)Cc2ccc3c(c2)OCO3)nc1-c1ccc2c(c1)CCN2C(=O)c1ccccc1OC(F)(F)F]

¹H NMR (400 MHz, DMSO-d6) δ ppm 12.28 (s, 1H), 8.19 (d, J=8.9 Hz, 1H), 7.74-7.60 (m, 2H), 7.60-7.49 (m, 4H), 6.93-6.83 (m, 2H), 6.79 (dd, J=8.0, 1.7 Hz, 1H), 5.99 (s, 2H), 3.81 (t, J=8.2 Hz, 2H), 3.65 (s, 2H), 3.20-3.11 (m, 2H), 2.46 (s, 3H); Method 1, retention time: 6.503 min; HRMS: m/z (M+Na)⁺=604.1140 (Calculated for C₂₉H₂₂F₃N₃NaO₅S=604.1124).

COMPOUND 115 was prepared according to the method described in Scheme 23 substituting 2-fluorobenzoyl chloride for 2-ethylbenzoyl chloride.

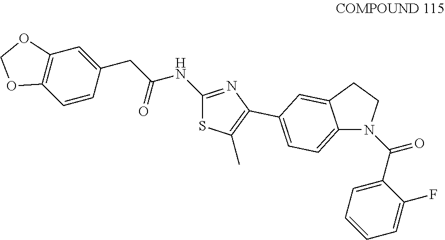 [SMILES: Cc1sc(NC(=O)Cc2ccc3c(c2)OCO3)nc1-c1ccc2c(c1)CCN2C(=O)c1ccccc1F]

¹H NMR (400 MHz, DMSO-d6) δ ppm 12.28 (s, 1H), 8.19 (d, J=8.4 Hz, 1H), 7.62-7.50 (m, 4H), 7.43-7.32 (m, 2H), 6.93-6.83 (m, 2H), 6.79 (dd, J=8.0, 1.8 Hz, 1H), 5.99 (s, 2H), 3.89 (t, J=8.3 Hz, 2H), 3.65 (s, 2H), 3.16 (t, J=8.3 Hz, 2H), 2.46 (s, 3H); Method 1, retention time: 6.157 min; HRMS: m/z (M+H)⁺=516.1378 (Calculated for C₂₈H₂₃FN₃O₄S=516.1388).

COMPOUND 116 was prepared according to the method described in Scheme 23 substituting 2-chlorobenzoyl chloride for 2-ethylbenzoyl chloride.

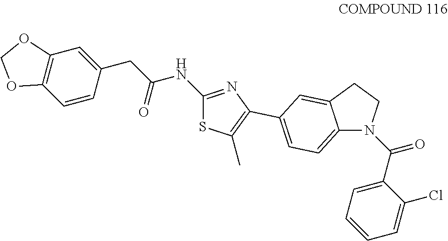 [SMILES: Cc1sc(NC(=O)Cc2ccc3c(c2)OCO3)nc1-c1ccc2c(c1)CCN2C(=O)c1ccccc1Cl]

¹H NMR (400 MHz, DMSO-d6) δ ppm 12.28 (s, 1H), 8.22 (d, J=8.8 Hz, 1H), 7.63-7.48 (m, 6H), 6.93-6.84 (m, 2H), 6.79 (dd, J=8.0, 1.7 Hz, 1H), 5.99 (s, 2H), 3.78 (t, J=8.3 Hz, 2H), 3.65 (s, 2H), 3.20-3.13 (m, 2H), 2.46 (s, 3H); Method 1, retention time: 6.207 min; HRMS: m/z (M+H)⁺=532.1074 (Calculated for C₂₈H₂₃ClN₃O₄S=532.1092).

COMPOUND 120 was prepared according to the method described in Scheme 23 substituting 2-methoxybenzoyl chloride for 2-ethylbenzoyl chloride.

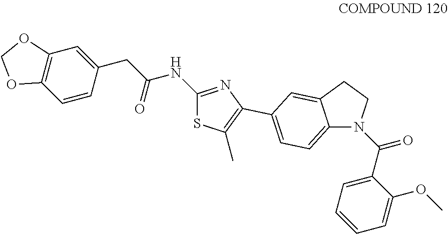 [SMILES: COc1ccccc1C(=O)N1CCc2cc(-c3nc(NC(=O)Cc4ccc5c(c4)OCO5)sc3C)ccc21]

Method 1, retention time: 5.969 min; HRMS: m/z (M+H)⁺=528.1592 (Calculated for C₂₉H₂₆N₃O₅S=528.1588).

COMPOUND 121 was prepared according to the method described in Scheme 23 substituting 2-(trifloromethyl)benzoyl chloride for 2-ethylbenzoyl chloride.

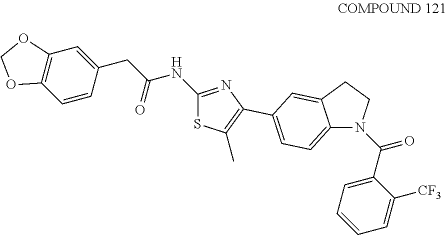 [SMILES: Cc1sc(NC(=O)Cc2ccc3c(c2)OCO3)nc1-c1ccc2c(c1)CCN2C(=O)c1ccccc1C(F)(F)F]

¹H NMR (400 MHz, DMSO-d6) δ ppm 12.28 (s, 1H), 8.20 (d, J=8.8 Hz, 1H), 7.93-7.86 (m, 1H), 7.87-7.78 (m, 1H), 7.76-7.67 (m, 2H), 7.54 (dtd, J=3.9, 1.8, 0.9 Hz, 2H), 6.91 (d, J=1.6 Hz, 1H), 6.87 (dd, J=8.0, 0.4 Hz, 1H), 6.79 (dd, J=8.0, 1.7 Hz, 1H), 5.99 (s, 2H), 3.64 (d, J=8.8 Hz, 4H), 3.22-3.06 (m, 2H), 2.46 (s, 3H); Method 1, retention time: 6.290 min; HRMS: m/z (M+H)⁺=566.1356 (Calculated for C₂₉H₂₃F₃N₃O₄S=566.1356).

COMPOUND 122 was prepared according to the method described in Scheme 4 substituting 2-(2-fluorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

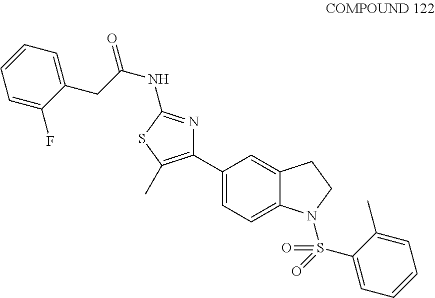 [SMILES: Cc1ccccc1S(=O)(=O)N1CCc2cc(-c3nc(NC(=O)Cc4ccccc4F)sc3C)ccc21]

¹H NMR (400 MHz, DMSO-d6) δ ppm 12.33 (s, 1H), 7.87 (dd, J=8.0, 1.4 Hz, 1H), 7.58 (td, J=7.5, 1.4 Hz, 1H), 7.50 (dd, J=1.8, 0.7 Hz, 1H), 7.48-7.43 (m, 2H), 7.44-7.41 (m, 1H), 7.41-7.36 (m, 1H), 7.35-7.30 (m, 1H), 7.28 (d, J=8.4 Hz, 1H), 7.22-7.15 (m, 2H), 4.02 (dd, J=8.9, 8.1 Hz, 2H), 3.87-3.80 (m, 2H), 3.10 (t, J=8.4 Hz, 2H), 2.55 (s, 3H), 2.41 (s, 3H); Method 1, retention time: 6.566 min; HRMS: m/z (M+H)⁺=522.1296 (Calculated for C₂₇H₂₅FN₃O₃S₂=522.1316).

COMPOUND 123 was prepared according to the method described in Scheme 4 substituting 2-(2-chlorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

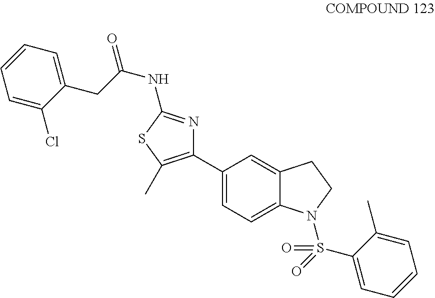 [SMILES: Cc1ccccc1S(=O)(=O)N1CCc2cc(-c3nc(NC(=O)Cc4ccccc4Cl)sc3C)ccc21]

¹H NMR (400 MHz, DMSO-d6) δ ppm 12.34 (s, 1H), 7.87 (dd, J=8.0, 1.4 Hz, 1H), 7.58 (td, J=7.5, 1.4 Hz, 1H), 7.50 (dd, J=1.8, 0.7 Hz, 1H), 7.48-7.39 (m, 5H), 7.35-7.30 (m, 2H), 7.28 (d, J=8.5 Hz, 1H), 4.07-3.98 (m, 2H), 3.94 (s, 2H), 3.15-3.06 (m, 2H), 2.55 (s, 3H), 2.41 (s, 3H); Method 1, retention time: 6.740 min; HRMS: m/z (M+H)⁺=538.1019 (Calculated for C₂₇H₂₅ClN₃O₃S₂=538.1020).

COMPOUND 124 was prepared according to the method described in Scheme 4 substituting 2-(3-fluorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

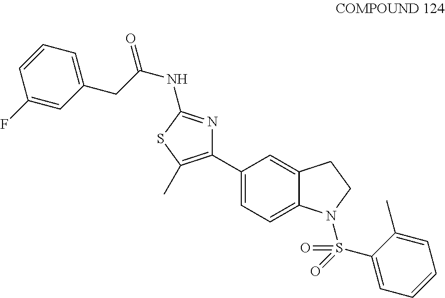 [SMILES: Cc1ccccc1S(=O)(=O)N1CCc2cc(-c3nc(NC(=O)Cc4cccc(F)c4)sc3C)ccc21]

¹H NMR (400 MHz, DMSO-d6) δ ppm 12.31 (s, 1H), 7.87 (dd, J=8.0, 1.4 Hz, 1H), 7.58 (td, J=7.5, 1.4 Hz, 1H), 7.49 (dt, J=2.0, 0.8 Hz, 1H), 7.48-7.34 (m, 4H), 7.28 (d, J=8.4 Hz, 1H), 7.20-7.13 (m, 2H), 7.10 (dddd, J=9.2, 8.3, 2.5, 1.1 Hz, 1H), 4.02 (dd, J=8.9, 8.1 Hz, 2H), 3.78 (s, 2H), 3.14-3.06 (m, 2H), 2.54 (s, 3H), 2.41 (s, 3H); Method 1, retention time: 6.632 min; HRMS: m/z (M+H)⁺=522.1335 (Calculated for C₂₇H₂₅FN₃O₃S₂=522.1316).

COMPOUND 125 was prepared according to the method described in Scheme 4 substituting 2-(3-chlorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

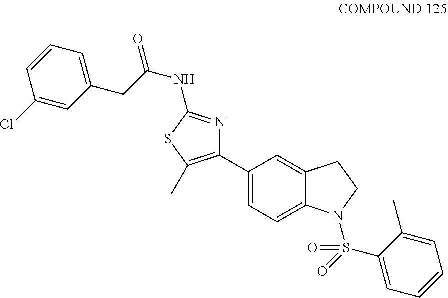 [SMILES: Cc1ccccc1S(=O)(=O)N1CCc2cc(-c3nc(NC(=O)Cc4cccc(Cl)c4)sc3C)ccc21]

¹H NMR (400 MHz, DMSO-d6) δ ppm 12.31 (s, 1H), 7.87 (dd, J=8.0, 1.4 Hz, 1H), 7.58 (td, J=7.5, 1.4 Hz, 1H), 7.51-7.48 (m, 1H), 7.48-7.31 (m, 6H), 7.31-7.25 (m, 2H), 4.02 (dd, J=8.9, 8.1 Hz, 2H), 3.78 (s, 2H), 3.15-3.05 (m, 2H), 2.54 (s, 3H), 2.41 (s, 3H); Method 1, retention time: 6.894 min; HRMS: m/z (M+Na)=560.0815 (Calculated for C₂₇H₂₄ClN₃NaO₃S₂=560.0840).

COMPOUND 126 was prepared according to the method described in Scheme 4 substituting 2-(4-fluorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

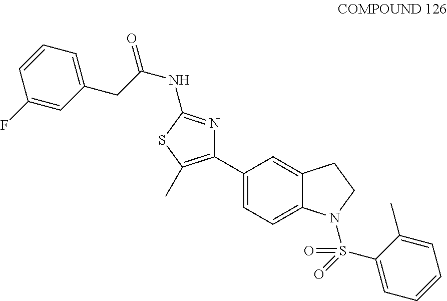 [SMILES: Cc1ccccc1S(=O)(=O)N1CCc2cc(-c3nc(NC(=O)Cc4cccc(F)c4)sc3C)ccc21]

¹H NMR (400 MHz, DMSO-d6) δ ppm 12.29 (s, 1H), 7.87 (dd, J=8.0, 1.4 Hz, 1H), 7.58 (td, J=7.5, 1.4 Hz, 1H), 7.49 (q, J=0.9 Hz, 1H), 7.48-7.39 (m, 3H), 7.39-7.32 (m, 2H), 7.27 (d, J=8.4 Hz, 1H), 7.20-7.12 (m, 2H), 4.02 (dd, J=8.9, 8.1 Hz, 2H), 3.74 (s, 2H), 3.09 (t, J=8.6 Hz, 2H), 2.54 (s, 3H), 2.41 (s, 3H); Method 1, retention time: 6.612 min; HRMS: m/z (M+H)⁺=522.1321 (Calculated for C₂₇H₂₅FN₃O₃S₂=522.1316).

COMPOUND 127 was prepared according to the method described in Scheme 4 substituting 2-(pyridin-2-yl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

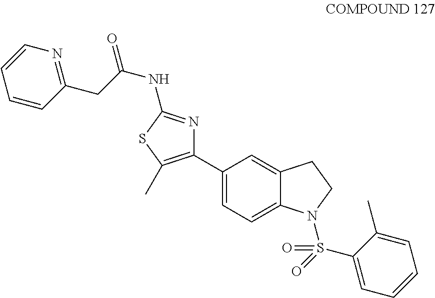 [SMILES: Cc1ccccc1S(=O)(=O)N1CCc2cc(-c3nc(NC(=O)Cc4ccccn4)sc3C)ccc21]

Method 1, retention time: 4.979 min; HRMS: m/z (M+H)⁺=505.1372 (Calculated for C₂₆H₂₅N₄O₃S₂=505.1363).

COMPOUND 128 was prepared according to the method described in Scheme 4 substituting 2-(o-tolyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

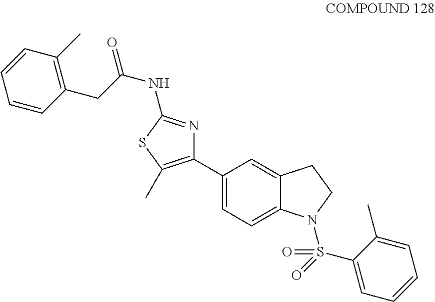 [SMILES: Cc1ccccc1CC(=O)Nc1nc(-c2ccc3c(c2)CCN3S(=O)(=O)c2ccccc2C)c(C)s1]

¹H NMR (400 MHz, DMSO-d6) δ ppm 12.28 (s, 1H), 7.87 (dd, J=7.9, 1.4 Hz, 1H), 7.58 (td, J=7.5, 1.4 Hz, 1H), 7.50 (q, J=0.9 Hz, 1H), 7.48-7.38 (m, 3H), 7.28 (d, J=8.4 Hz, 1H), 7.24 (dd, J=5.7, 2.6 Hz, 1H), 7.19-7.12 (m, 3H), 4.07-3.97 (m, 2H), 3.78 (s, 2H), 3.10 (t, J=8.3 Hz, 2H), 2.54 (s, 3H), 2.41 (s, 3H), 2.27 (s, 3H); Method 1, retention time: 6.809 min; HRMS: m/z (M+H)⁺=510.1581 (Calculated for C₂₈H₂₈N₃O₃S₂=518.1567).

COMPOUND 129 was prepared according to the method described in Scheme 4 substituting 2-(2-trifluoromethyl)phenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

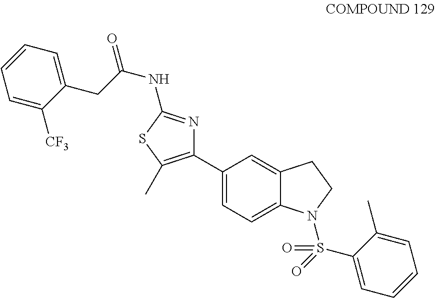 [SMILES: Cc1ccccc1S(=O)(=O)N1CCc2cc(-c3nc(NC(=O)Cc4ccccc4C(F)(F)F)sc3C)ccc21]

¹H NMR (400 MHz, DMSO-d6) δ ppm 12.33 (s, 1H), 7.88 (dd, J=8.0, 1.4 Hz, 1H), 7.74-7.70 (m, 1H), 7.66 (t, J=7.5 Hz, 1H), 7.58 (td, J=7.5, 1.4 Hz, 1H), 7.55-7.48 (m, 3H), 7.48-7.39 (m, 3H), 7.28 (d, J=8.4 Hz, 1H), 4.08-3.98 (m, 4H), 3.10 (t, J=8.5 Hz, 2H), 2.55 (s, 3H), 2.41 (s, 3H); Method 1, retention time: 6.851 min; HRMS: m/z (M+H)⁺=572.1264 (Calculated for C₂₈H₂₅F₃N₃O₃S₂=572.1284).

COMPOUND 130 was prepared according to the method described in Scheme 4 substituting 2-(pyridin-3-yl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

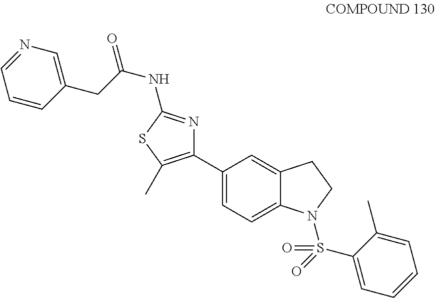 [SMILES: Cc1ccccc1S(=O)(=O)N1CCc2cc(-c3nc(NC(=O)Cc4cccnc4)sc3C)ccc21]

¹H NMR (400 MHz, DMSO-d6) δ 12.40 (s, 1H), 8.68 (d, J=2.1 Hz, 1H), 8.64 (dd, J=5.2, 1.5 Hz, 1H), 8.07 (d, J=7.9 Hz, 1H), 7.87 (dd, J=7.9, 1.4 Hz, 1H), 7.66 (dd, J=7.9, 5.2 Hz, 1H), 7.58 (td, J=7.5, 1.4 Hz, 1H), 7.52-7.48 (m, 1H), 7.48-7.39 (m, 3H), 7.28 (d, J=8.4 Hz, 1H), 4.02 (dd, J=8.9, 8.1 Hz, 2H), 3.93 (s, 2H), 3.15-3.06 (m, 2H), 2.55 (s, 3H), 2.41 (s, 3H); Method 1, retention time: 4.777 min; HRMS: m/z (M+H)⁺=505.1353 (Calculated for C₂₆H₂₅N₄O₃S₂=505.1363).

COMPOUND 131 was prepared according to the method described in Scheme 4 substituting 2-(m-tolyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

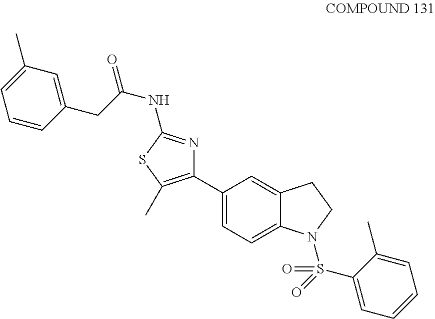 [SMILES: Cc1cccc(CC(=O)Nc2nc(-c3ccc4c(c3)CCN4S(=O)(=O)c3ccccc3C)c(C)s2)c1]

¹H NMR (400 MHz, DMSO-d6) δ 12.27 (s, 1H), 7.87 (dd, J=8.0, 1.4 Hz, 1H), 7.58 (td, J=7.5, 1.4 Hz, 1H), 7.49 (dd, J=1.8, 0.7 Hz, 1H), 7.48-7.39 (m, 3H), 7.27 (d, J=8.4 Hz, 1H), 7.21 (t, J=7.5 Hz, 1H), 7.15-7.09 (m, 2H), 7.07 (dd, J=8.0, 1.5 Hz, 1H), 4.01 (dd, J=8.9, 8.1 Hz, 2H), 3.69 (s, 2H), 3.15-3.04 (m, 2H), 2.54 (s, 3H), 2.41 (s, 3H), 2.29 (d, J=0.8 Hz, 3H); Method 1, retention time: 6.839 min; HRMS: m/z (M+H)⁺=518.1571 (Calculated for C₂₈H₂₈N₃O₃S₂=518.1567).

COMPOUND 132 was prepared according to the method described in Scheme 4 substituting 2-(3-(trifluoromethyl)phenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

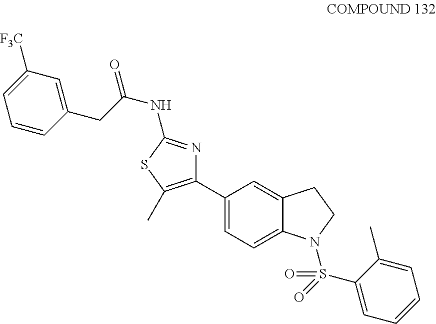 [SMILES: Cc1ccccc1S(=O)(=O)N1CCc2cc(-c3nc(NC(=O)Cc4cccc(C(F)(F)F)c4)sc3C)ccc21]

¹H NMR (400 MHz, DMSO-d6) δ ppm 12.35 (s, 1H), 7.87 (dd, J=8.1, 1.4 Hz, 1H), 7.71 (s, 1H), 7.63 (d, J=5.4 Hz, 2H), 7.61-7.55 (m, 2H), 7.50 (s, 1H), 7.48-7.41 (m, 3H), 7.28 (d, J=8.4 Hz, 1H), 4.02 (t, J=8.5 Hz, 2H), 3.89 (s, 2H), 3.10 (t, J=8.4 Hz, 2H), 2.55 (d, J=1.3 Hz, 3H), 2.41 (d, J=1.5 Hz, 3H); Method 1, retention time: 6.949 min; HRMS: m/z (M+H)⁺=572.1288 (Calculated for C₂₈H₂₅F₃N₃O₃S₂=572.1284).

COMPOUND 133 was prepared according to the method described in Scheme 4 substituting 2-(p-tolyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

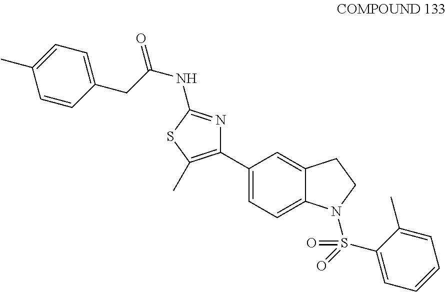 [SMILES: Cc1ccc(CC(=O)Nc2nc(-c3ccc4c(c3)CCN4S(=O)(=O)c3ccccc3C)c(C)s2)cc1]

¹H NMR (400 MHz, DMSO-d6) δ 12.25 (s, 1H), 7.87 (dd, J=8.0, 1.4 Hz, 1H), 7.58 (td, J=7.5, 1.4 Hz, 1H), 7.49 (d, J=1.7 Hz, 1H), 7.48-7.38 (m, 3H), 7.27 (d, J=8.4 Hz, 1H), 7.23-7.18 (m, 2H), 7.15-7.10 (m, 2H), 4.01 (t, J=8.5 Hz, 2H), 3.68 (s, 2H), 3.09 (t, J=8.4 Hz, 2H), 2.54 (s, 3H), 2.40 (s, 3H), 2.27 (s, 3H); Method 1, retention time: 6.838 min; HRMS: m/z (M+H)⁺=518.1541 (Calculated for C₂₈H₂₈N₃O₃S₂=518.1567).

COMPOUND 134 was prepared according to the method described in Scheme 4 substituting 2-(4-(trifluoromethyl)phenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

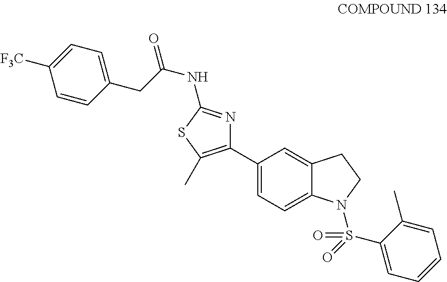 [SMILES: Cc1ccccc1S(=O)(=O)N1CCc2cc(-c3nc(NC(=O)Cc4ccc(C(F)(F)F)cc4)sc3C)ccc21]

¹H NMR (400 MHz, DMSO-d6) δ ppm 12.37 (s, 1H), 7.87 (dd, J=8.0, 1.4 Hz, 1H), 7.71 (d, J=8.1 Hz, 1H), 7.59-7.53 (m, 4H), 7.49 (d, J=1.7 Hz, 1H), 7.46-7.43 (m, 1H), 7.42 (d, J=1.5 Hz, 1H), 7.41-7.36 (m, 1H), 7.28 (d, J=8.4 Hz, 1H), 4.02 (t, J=8.4 Hz, 2H), 3.88 (d, J=1.0 Hz, 2H), 3.10 (t, J=8.4 Hz, 2H), 2.54 (s, 3H), 2.41 (s, 3H); Method 1, retention time: 6.959 min; HRMS: m/z (M+H)⁺=572.1273 (Calculated for C₂₈H₂₅F₃N₃O₃S₂=572.1284).

COMPOUND 135 was prepared according to the method described in Scheme 4 substituting 2-(4-chlorophenyl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

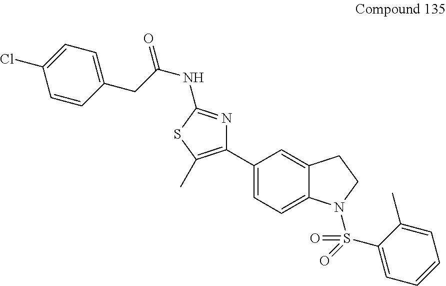 [SMILES: Cc1ccccc1S(=O)(=O)N1CCc2cc(-c3nc(NC(=O)Cc4ccc(Cl)cc4)sc3C)ccc21]

¹H NMR (400 MHz, DMSO-d6) δ ppm 12.30 (s, 1H), 7.87 (dd, J=8.0, 1.4 Hz, 1H), 7.58 (td, J=7.5, 1.4 Hz, 1H), 7.49 (d, J=1.7 Hz, 1H), 7.47-7.43 (m, 2H), 7.43-7.32 (m, 5H), 7.27 (d, J=8.4 Hz, 1H), 4.01 (t, J=8.5 Hz, 2H), 3.75 (s, 2H), 3.09 (t, J=8.4 Hz, 2H), 2.54 (s, 3H), 2.41 (s, 3H); Method 1, retention time: 6.898 min; HRMS: m/z (M+H)⁺=538.0996 (Calculated for C₂₇H₂₅ClN₃O₃S₂=538.1020).

COMPOUND 136 was prepared according to the method described in Scheme 4 substituting 2-(pyridin-4-yl)acetic acid for 5-(3-nitrophenyl)furan-2-carboxylic acid and substituting 5-methyl-4-(1-(o-tolylsulfonyl)indolin-5-yl)thiazol-2-amine for 5-phenylthiazol-2-amine.

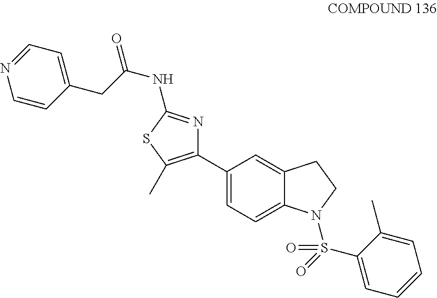 [SMILES: Cc1ccccc1S(=O)(=O)N1CCc2cc(-c3nc(NC(=O)Cc4ccncc4)sc3C)ccc21]

¹H NMR (400 MHz, DMSO-d6) δ 12.44 (s, 1H), 8.72-8.68 (m, 2H), 7.87 (dd, J=8.0, 1.4 Hz, 1H), 7.68 (d, J=5.4 Hz, 2H), 7.58 (td, J=7.5, 1.4 Hz, 1H), 7.49 (d, J=1.7 Hz, 1H), 7.48-7.41 (m, 3H), 7.28 (d, J=8.4 Hz, 1H), 4.06-3.97 (m, 4H), 3.10 (t, J=8.4 Hz, 2H), 2.55 (s, 3H), 2.42 (s, 3H); Method 1, retention time: 4.760 min; HRMS: m/z (M+H)⁺=505.1359 (Calculated for C₂₆H₂₅N₄O₃S₂=505.1363).

COMPOUND 138 was prepared according to the method described in Scheme 4 substituting 2-isopropylbenzoic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

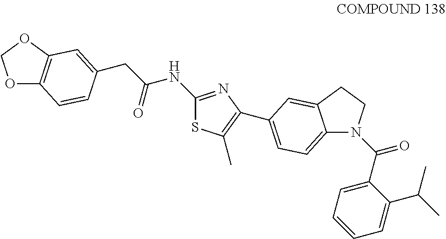 [SMILES: Cc1sc(NC(=O)Cc2ccc3c(c2)OCO3)nc1-c1ccc2c(c1)CCN2C(=O)c1ccccc1C(C)C]

¹H NMR (400 MHz, DMSO-d6) δ 12.28 (s, 1H), 8.25 (d, J=8.8 Hz, 1H), 7.53 (dd, J=4.7, 3.0 Hz, 2H), 7.46 (ddd, J=10.1, 7.2, 1.6 Hz, 2H), 7.32 (qd, J=6.7, 1.4 Hz, 2H), 6.93-6.84 (m, 2H), 6.79 (dd, J=8.0, 1.7 Hz, 1H), 5.99 (s, 2H), 3.75-3.58 (m, 4H), 3.14 (q, J=8.0 Hz, 2H), 2.46 (s, 3H), 2.34 (s, 1H), 1.25-1.19 (m, 6H); Method 1, retention time: 6.670 min; HRMS: m/z (M+H)⁺=540.1952 (Calculated for C₃₁H₃₀N₃O₄S=540.1952).

COMPOUND 139 was prepared according to the method described in Scheme 4 substituting 2-methylnicotinic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

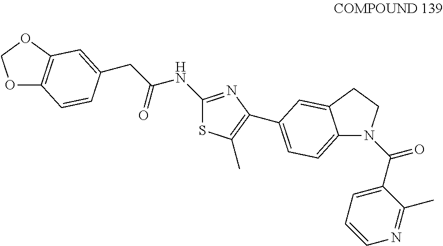 [SMILES: Cc1ncccc1C(=O)N1CCc2cc(-c3nc(NC(=O)Cc4ccc5c(c4)OCO5)sc3C)ccc21]

¹H NMR (400 MHz, DMSO-d6) δ 12.28 (s, 1H), 8.63 (d, J=5.0 Hz, 1H), 8.23 (d, J=8.6 Hz, 1H), 8.03 (d, J=7.7 Hz, 1H), 7.58-7.52 (m, 2H), 7.49 (t, J=6.5 Hz, 1H), 6.94-6.84 (m, 2H), 6.83-6.75 (m, 1H), 5.99 (s, 2H), 3.81 (t, J=8.3 Hz, 2H), 3.65 (s, 2H), 3.16 (t, J=8.4 Hz, 2H), 2.54 (s, 3H), 2.46 (s, 3H); Method 1, retention time: 4.604 min; HRMS: m/z (M+H)⁺=513.1605 (Calculated for C₂₈H₂₅N₄O₄S=513.1591).

COMPOUND 140 was prepared according to the method described in Scheme 4 substituting 4-methylnicotinic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

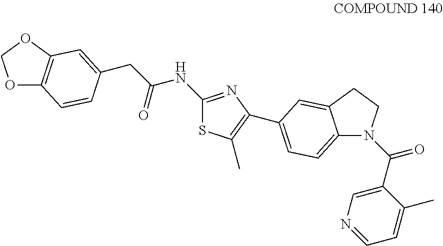 [SMILES: Cc1ccncc1C(=O)N1CCc2cc(-c3nc(NC(=O)Cc4ccc5c(c4)OCO5)sc3C)ccc21]

¹H NMR (400 MHz, DMSO-d6) δ 12.28 (s, 1H), 8.69 (s, 1H), 8.60 (d, J=5.3 Hz, 1H), 8.24 (d, J=8.7 Hz, 1H), 7.60-7.49 (m, 3H), 6.94-6.83 (m, 2H), 6.82-6.75 (m, 1H), 5.99 (s, 2H), 3.82 (t, J=8.3 Hz, 2H), 3.65 (s, 2H), 3.16 (t, J=8.3 Hz, 2H), 2.48-2.43 (m, 3H), 2.39 (s, 3H); Method 1, retention time: 4.648 min; HRMS: m/z (M+H)⁺=513.1590 (Calculated for C₂₈H₂₅N₄O₄S=513.1591).

COMPOUND 141 was prepared according to the method described in Scheme 4 substituting 3-methylnicotinic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

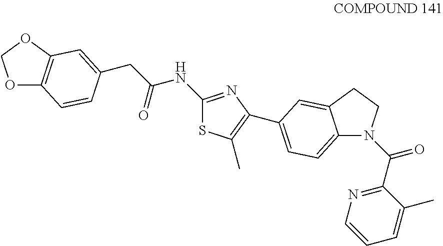 [SMILES: Cc1cccnc1C(=O)N1CCc2cc(-c3nc(NC(=O)Cc4ccc5c(c4)OCO5)sc3C)ccc21]

¹H NMR (400 MHz, DMSO-d6) δ 12.27 (s, 1H), 8.47 (ddd, J=4.8, 1.4, 0.7 Hz, 1H), 8.24 (d, J=8.8 Hz, 1H), 7.81 (ddd, J=7.9, 1.7, 0.9 Hz, 1H), 7.58-7.52 (m, 2H), 7.43 (dd, J=7.8, 4.8 Hz, 1H), 6.94-6.83 (m, 2H), 6.79 (dd, J=8.0, 1.7 Hz, 1H), 5.99 (s, 2H), 3.82 (dd, J=9.0, 7.9 Hz, 2H), 3.65 (s, 2H), 3.20-3.11 (m, 2H), 2.46 (s, 3H), 2.33 (s, 3H); Method 1, retention time: 5.525 min; HRMS: m/z (M+H)⁺=513.1604 (Calculated for C₂₈H₂₅N₄O₄S=513.1591).

COMPOUND 157 was prepared according to the method described in Scheme 4 substituting 2,5-dimethyloxazole-4-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

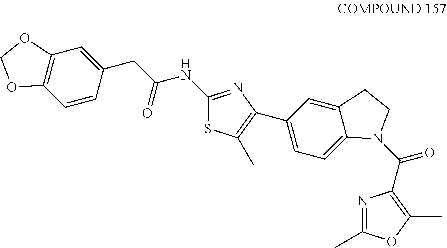 [SMILES: Cc1nc(C(=O)N2CCc3cc(-c4nc(NC(=O)Cc5ccc6c(c5)OCO6)sc4C)ccc32)c(C)o1]

¹H NMR (400 MHz, DMSO-d6) δ 12.25 (s, 1H), 8.15 (s, 1H), 7.53 (dq, J=1.7, 0.9 Hz, 1H), 7.48 (dd, J=8.5, 1.8 Hz, 1H), 6.90 (d, J=1.7 Hz, 1H), 6.86 (d, J=7.9 Hz, 1H), 6.79 (dd, J=8.0, 1.7 Hz, 1H), 5.99 (s, 2H), 4.52-4.42 (m, 2H), 3.65 (s, 2H), 3.19 (t, J=8.4 Hz, 2H), 2.52 (s, 3H), 2.45 (s, 3H), 2.43 (s, 3H); Method 1, retention time: 6.007 min; HRMS: m/z (M+H)⁺=517.1519 (Calculated for C₂₇H₂₅N₄O₅S=517.1540).

COMPOUND 158 was prepared according to the method described in Scheme 4 substituting 5-isopropylisoxazole-4-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

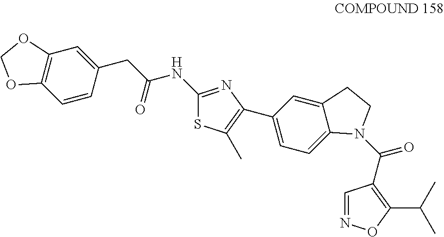 [SMILES: Cc1sc(NC(=O)Cc2ccc3c(c2)OCO3)nc1-c1ccc2c(c1)CCN2C(=O)c1cnoc1C(C)C]

Method 1, retention time: 6.923 min; HRMS: m/z (M+H)⁺=531.1717 (Calculated for C₂₈H₂₇N₄O₅S=531.1697).

COMPOUND 159 was prepared according to the method described in Scheme 4 substituting 2-methylfuran-3-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

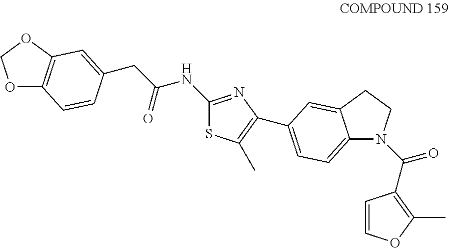 [SMILES: Cc1occc1C(=O)N1CCc2cc(-c3nc(NC(=O)Cc4ccc5c(c4)OCO5)sc3C)ccc21]

¹H NMR (400 MHz, DMSO-d6) δ 12.25 (s, 1H), 7.97 (s, 1H), 7.62 (d, J=2.0 Hz, 1H), 7.53 (dd, J=1.7, 0.8 Hz, 1H), 7.47 (dd, J=8.6, 1.8 Hz, 1H), 6.90 (dd, J=1.7, 0.5 Hz, 1H), 6.86 (dd, J=7.9, 0.4 Hz, 1H), 6.79 (dd, J=8.0, 1.7 Hz, 2H), 5.99 (s, 2H), 4.16 (dd, J=8.9, 7.9 Hz, 2H), 3.65 (s, 2H), 3.16 (t, J=8.3 Hz, 2H), 2.45 (s, 3H), 2.40 (s, 3H); Method 1, retention time: 5.984 min; HRMS: m/z (M+H)⁺=502.1410 (Calculated for C₂₇H₂₄N₃O₅S=502.1431).

COMPOUND 160 was prepared according to the method described in Scheme 4 substituting 5-methyl-3-phenylisoxazole-4-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

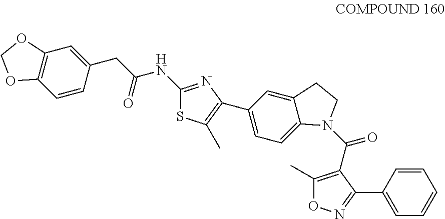 [SMILES: Cc1onc(-c2ccccc2)c1C(=O)N1CCc2cc(-c3nc(NC(=O)Cc4ccc5c(c4)OCO5)sc3C)ccc21]

¹H NMR (400 MHz, DMSO-d6) δ 12.26 (s, 1H), 8.18 (s, 1H), 7.65 (s, 2H), 7.50 (s, 5H), 6.90 (d, J=1.6 Hz, 1H), 6.86 (d, J=7.9 Hz, 1H), 6.78 (dd, J=8.0, 1.7 Hz, 1H), 5.98 (s, 2H), 3.78 (d, J=10.0 Hz, 2H), 3.64 (s, 2H), 3.06 (t, J=8.2 Hz, 2H), 2.55 (s, 3H), 2.44 (s, 3H); Method 1, retention time: 6.276 min; HRMS: m/z (M+H)⁺=579.1691 (Calculated for C₃₂H₂₇N₄O₅S=579.1697).

COMPOUND 161 was prepared according to the method described in Scheme 4 substituting 1-methyl-1H-imidazole-5-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

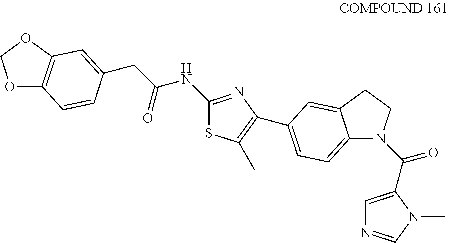 [SMILES: Cc1sc(NC(=O)Cc2ccc3c(c2)OCO3)nc1-c1ccc2c(c1)CCN2C(=O)c1cncn1C]

¹H NMR (400 MHz, DMSO-d6) δ 12.26 (s, 1H), 8.57 (s, 1H), 8.09 (d, J=8.5 Hz, 1H), 7.95 (s, 1H), 7.57 (dq, J=1.6, 0.9 Hz, 1H), 7.52 (dd, J=8.4, 1.9 Hz, 1H), 6.93-6.89 (m, 1H), 6.87 (dd, J=7.9, 0.4 Hz, 1H), 6.79 (dd, J=8.0, 1.7 Hz, 1H), 5.99 (s, 2H), 4.36 (dd, J=8.8, 7.8 Hz, 2H), 3.89 (d, J=0.6 Hz, 3H), 3.65 (s, 2H), 3.21 (t, J=8.3 Hz, 2H), 2.46 (s, 3H); Method 1, retention time: 4.453 min; HRMS: m/z (M+H)⁺=502.1542 (Calculated for C₂₆H₂₄N₅O₄S=502.1544).

COMPOUND 162 was prepared according to the method described in Scheme 4 substituting 3-methyl-5,6-dihydro-1,4-dioxine-2-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

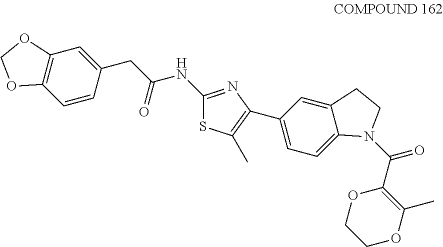 [SMILES: CC1=C(C(=O)N2CCc3cc(-c4nc(NC(=O)Cc5ccc6c(c5)OCO6)sc4C)ccc32)OCCO1]

¹H NMR (400 MHz, DMSO-d6) δ 12.25 (s, 1H), 7.85 (s, 1H), 7.53-7.48 (m, 1H), 7.46 (dd, J=8.4, 1.9 Hz, 1H), 6.90 (d, J=1.6 Hz, 1H), 6.86 (d, J=7.9 Hz, 1H), 6.78 (dd, J=8.0, 1.7 Hz, 1H), 5.98 (s, 2H), 4.19-4.14 (m, 2H), 4.11 (t, J=8.4 Hz, 2H), 4.08-4.03 (m, 2H), 3.64 (s, 2H), 3.14 (t, J=8.3 Hz, 2H), 2.44 (s, 3H), 1.89 (s, 3H); Method 1, retention time: 5.793 min; HRMS: m/z (M+H)⁺=520.1547 (Calculated for C₂₇H₂₆N₃O₆S=520.1537).

COMPOUND 180 was prepared according to the method described in Scheme 4 substituting 2-(dimethylamino)acetic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

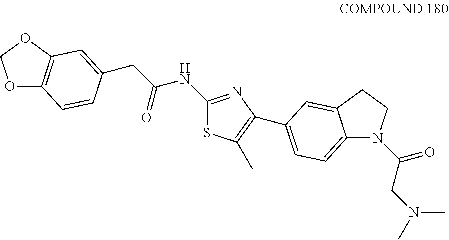 [SMILES: Cc1sc(NC(=O)Cc2ccc3c(c2)OCO3)nc1-c1ccc2c(c1)CCN2C(=O)CN(C)C]

¹H NMR (400 MHz, DMSO-d6) δ 12.24 (s, 1H), 8.11 (d, J=8.4 Hz, 1H), 7.57 (dd, J=1.6, 0.7 Hz, 1H), 7.52 (ddd, J=8.4, 1.8, 0.8 Hz, 1H), 6.90 (d, J=1.6 Hz, 1H), 6.87 (d, J=7.9 Hz, 1H), 6.78 (dd, J=8.0, 1.7 Hz, 1H), 5.99 (s, 2H), 4.35 (d, J=2.8 Hz, 2H), 4.08 (t, J=8.4 Hz, 2H), 3.65 (s, 2H), 3.27 (t, J=8.5 Hz, 2H), 2.94-2.86 (m, 6H), 2.45 (s, 3H); Method 1, retention time: 4.336 min; HRMS: m/z (M+H)⁺=479.1760 (Calculated for C₂₅H₂₇N₄O₄S=479.1748).

COMPOUND 181 was prepared according to the method described in Scheme 4 substituting 4-methyloxazole-5-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

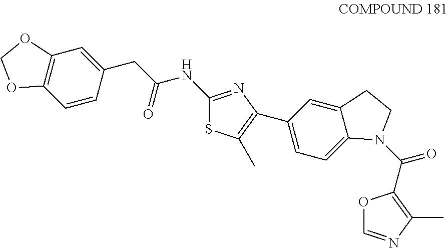 [SMILES: Cc1ncoc1C(=O)N1CCc2cc(-c3nc(NC(=O)Cc4ccc5c(c4)OCO5)sc3C)ccc21]

¹H NMR (400 MHz, DMSO-d6) δ 12.26 (s, 1H), 8.51 (d, J=0.6 Hz, 1H), 8.09 (s, 1H), 7.56 (dd, J=1.8, 0.8 Hz, 1H), 7.50 (dd, J=8.5, 1.9 Hz, 1H), 6.90 (d, J=1.7 Hz, 1H), 6.87 (d, J=7.9 Hz, 1H), 6.79 (dd, J=8.0, 1.7 Hz, 1H), 5.99 (s, 2H), 4.40 (t, J=8.3 Hz, 2H), 3.65 (s, 2H), 3.28-3.20 (m, 2H), 2.46 (s, 3H), 2.41 (s, 3H); Method 1, retention time: 5.544 min; HRMS: m/z (M+H)⁺=503.1396 (Calculated for C₂₆H₂₃N₄O₅S=503.1384).

COMPOUND 182 was prepared according to the method described in Scheme 4 substituting 3-methylthiophene-2-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

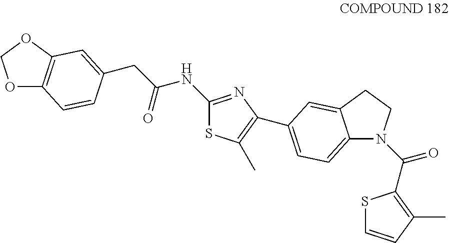 [SMILES: Cc1ccsc1C(=O)N1CCc2cc(-c3nc(NC(=O)Cc4ccc5c(c4)OCO5)sc3C)ccc21]

¹H NMR (400 MHz, DMSO-d6) δ 12.24 (s, 1H), 7.65 (d, J=5.0 Hz, 1H), 7.64-7.54 (m, 1H), 7.54-7.50 (m, 1H), 7.42 (dd, J=8.4, 1.8 Hz, 1H), 7.00 (d, J=4.9 Hz, 1H), 6.88 (d, J=1.6 Hz, 1H), 6.85 (d, J=7.9 Hz, 1H), 6.77 (dd, J=8.0, 1.7 Hz, 1H), 5.97 (s, 2H), 4.07 (dd, J=8.8, 7.8 Hz, 2H), 3.63 (s, 2H), 3.15 (t, J=8.3 Hz, 2H), 2.42 (s, 3H), 2.23 (s, 3H).; Method 1, retention time: 6.155 min; HRMS: m/z (M+H)=518.1222 (Calculated for C₂₇H₂₄N₃O₄S₂=518.1203).

COMPOUND 183 was prepared according to the method described in Scheme 4 substituting 3,5-dimethylisoxazole-4-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

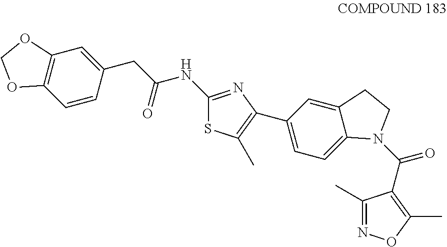 [SMILES: Cc1noc(C)c1C(=O)N1CCc2cc(-c3nc(NC(=O)Cc4ccc5c(c4)OCO5)sc3C)ccc21]

¹H NMR (400 MHz, DMSO-d6) δ 12.25 (s, 1H), 7.92 (s, br, 1H), 7.53 (d, J=1.7 Hz, 1H), 7.46 (d, J=8.4 Hz, 1H), 6.88 (d, J=1.6 Hz, 1H), 6.85 (d, J=7.9 Hz, 1H), 6.77 (dd, J=7.9, 1.7 Hz, 1H), 5.97 (s, 2H), 4.00 (t, J=8.2 Hz, 2H), 3.63 (s, 2H), 3.24-3.06 (m, 2H), 2.42 (d, J=2.3 Hz, 6H), 2.21 (s, 3H); Method 1, retention time: 5.544 min; HRMS: m/z (M+H)⁺=517.1533 (Calculated for C₂₇H₂₅N₄O₅S=517.1540).

COMPOUND 184 was prepared according to the method described in Scheme 4 substituting 1-methyl-1H-pyrrole-2-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

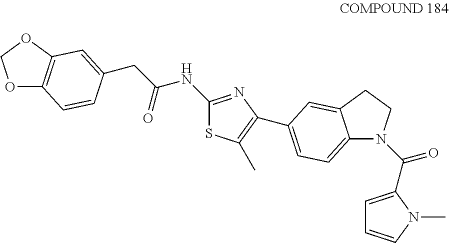 [SMILES: Cc1sc(NC(=O)Cc2ccc3c(c2)OCO3)nc1-c1ccc2c(c1)CCN2C(=O)c1cccn1C]

¹H NMR (400 MHz, DMSO-d6) δ 12.25 (s, 1H), 7.95 (d, J=8.4 Hz, 1H), 7.52 (dd, J=2.0, 0.8 Hz, 1H), 7.46 (dd, J=8.4, 1.9 Hz, 1H), 7.00 (ddd, J=2.6, 1.7, 0.4 Hz, 1H), 6.90 (dd, J=1.7, 0.5 Hz, 1H), 6.87 (dd, J=7.9, 0.4 Hz, 1H), 6.79 (dd, J=7.9, 1.7 Hz, 1H), 6.70 (dd, J=3.9, 1.7 Hz, 1H), 6.11 (dd, J=3.9, 2.5 Hz, 1H), 5.99 (s, 2H), 4.32 (t, J=8.3 Hz, 2H), 3.77 (d, J=0.4 Hz, 3H), 3.65 (s, 2H), 3.20-3.12 (m, 2H), 2.45 (s, 3H); Method 1, retention time: 6.007 min; HRMS: m/z (M+H)⁺=501.1599 (Calculated for C₂₇H₂₅N₄O₄S=501.1591).

COMPOUND 185 was prepared according to the method described in Scheme 4 substituting 1-methyl-1H-pyrazole-5-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

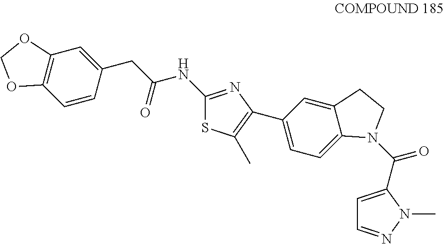 [SMILES: Cc1sc(NC(=O)Cc2ccc3c(c2)OCO3)nc1-c1ccc2c(c1)CCN2C(=O)c1ccnn1C]

¹H NMR (400 MHz, DMSO-d6) δ 12.27 (s, 1H), 8.14 (s, 1H), 7.56 (s, 1H), 7.54 (d, J=2.0 Hz, 1H), 7.51 (d, J=8.4 Hz, 1H), 6.90 (d, J=1.7 Hz, 1H), 6.87 (d, J=7.9 Hz, 1H), 6.79 (dd, J=7.9, 1.8 Hz, 2H), 5.99 (s, 2H), 4.24 (t, J=8.3 Hz, 2H), 3.95 (s, 3H), 3.65 (s, 2H), 3.18 (dd, J=9.4, 6.9 Hz, 2H), 2.45 (s, 3H); Method 1, retention time: 5.472 min; HRMS: m/z (M+H)⁺=502.1531 (Calculated for C₂₆H₂₄N₅O₄S=502.1544).

COMPOUND 186 was prepared according to the method described in Scheme 4 substituting 3-chlorothiophene-2-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

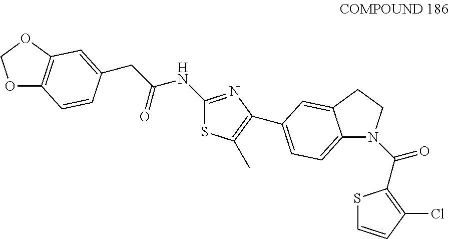 [SMILES: Cc1sc(NC(=O)Cc2ccc3c(c2)OCO3)nc1-c1ccc2c(c1)CCN2C(=O)c1sccc1Cl]

Method 1, retention time: 6.214 min; HRMS: m/z (M+H)⁺=538.0647 (Calculated for C₂₆H₂₁ClN₃O₄S₂=538.0657).

COMPOUND 187 was prepared according to the method described in Scheme 4 substituting 4-chloro-1-methyl-1H-pyrazole-5-carboxylic acid for 5-(3-nitrophenyl)furan-2-carboxylic acid and substituting 2-(benzo[d][1,3]dioxol-5-yl)-N-(4-(indolin-5-yl)-5-methylthiazol-2-yl)acetamide for 5-phenylthiazol-2-amine.

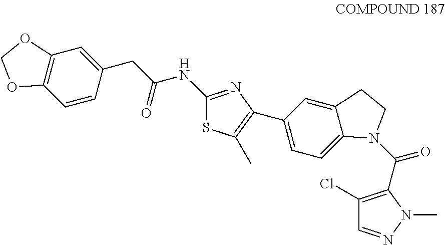 [SMILES: Cc1sc(NC(=O)Cc2ccc3c(c2)OCO3)nc1-c1ccc2c(c1)CCN2C(=O)c1c(Cl)cnn1C]

¹H NMR (400 MHz, DMSO-d6) δ 12.29 (s, 1H), 8.17 (d, J=8.4 Hz, 1H), 7.70 (s, 1H), 7.61-7.52 (m, 2H), 6.90 (d, J=1.6 Hz, 1H), 6.86 (d, J=7.9 Hz, 1H), 6.79 (dd, J=8.0, 1.7 Hz, 1H), 5.99 (s, 2H), 4.07 (d, J=9.1 Hz, 2H), 3.87 (s, 3H), 3.65 (s, 2H), 3.22 (t, J=8.3 Hz, 2H), 2.46 (s, 3H); Method 1, retention time: 5.891 min; HRMS: m/z (M+H)⁺=537.11838 (Calculated for C₂₆H₂₃ClN₅O₄S=537.1183).

COMPOUND 195 was prepared according to the method described in Scheme 4 substituting cyclopropanecarboxylic acid for 5-(3-nitrophenyl)furan-2-carboxylic acid and substituting N-(4-(indolin-5-yl)-5-methylthiazol-2-yl)-2-(4-methoxyphenyl)acetamide for 5-phenylthiazol-2-amine.

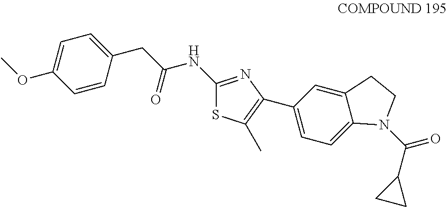 [SMILES: COc1ccc(CC(=O)Nc2nc(-c3ccc4c(c3)CCN4C(=O)C3CC3)c(C)s2)cc1]

¹H NMR (400 MHz, DMSO-d6) δ 12.25 (s, 1H), 8.06 (d, J=8.4 Hz, 1H), 7.49 (d, J=1.8 Hz, 1H), 7.46-7.38 (m, 1H), 7.28-7.21 (m, 2H), 6.93-6.86 (m, 2H), 4.33 (s, 2H), 3.73 (d, J=0.4 Hz, 3H), 3.66 (s, 2H), 3.24 (dd, J=15.8, 7.1 Hz, 2H), 2.43 (s, 3H), 1.96 (s, 1H), 0.93-0.81 (m, 4H); Method 1, retention time: 5.795 min; HRMS: m/z (M+H)⁺=448.1697 (Calculated for C₂₅H₂₆N₃O₃S=448.1689).

COMPOUND 196 was prepared according to the method described in Scheme 4 substituting 2-methylnicotinic acid for 5-(3-nitrophenyl)furan-2-carboxylic acid and substituting N-(4-(indolin-5-yl)-5-methylthiazol-2-yl)-2-(4-methoxyphenyl)acetamide for 5-phenylthiazol-2-amine.

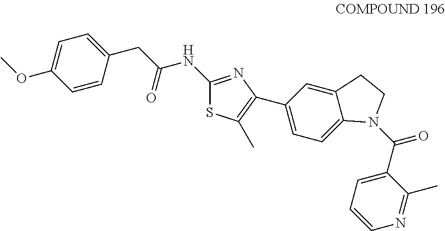

¹H NMR (400 MHz, DMSO-d6) δ 12.27 (s, 1H), 8.64-8.57 (m, 1H), 8.21 (d, J=8.8 Hz, 1H), 7.98 (d, J=7.6 Hz, 1H), 7.57-7.49 (m, 2H), 7.45 (dd, J=7.7, 5.2 Hz, 1H), 7.26-7.20 (m, 2H), 6.93-6.83 (m, 2H), 3.79 (t, J=8.3 Hz, 2H), 3.71 (s, 3H), 3.64 (s, 2H), 3.13 (t, J=8.4 Hz, 2H), 2.51 (s, 3H), 2.43 (s, 3H); Method 1, retention time: 4.654 min; HRMS: m/z (M+H)⁺=499.1800 (Calculated for C₂₈H₂₇N₄O₃S=499.1798).

COMPOUND 197 was prepared according to the method described in Scheme 4 substituting 1-methyl-1H-pyrazole-5-carboxylic acid for 5-(3-nitrophenyl)furan-2-carboxylic acid and substituting N-(4-(indolin-5-yl)-5-methylthiazol-2-yl)-2-(4-methoxyphenyl)acetamide for 5-phenylthiazol-2-amine.

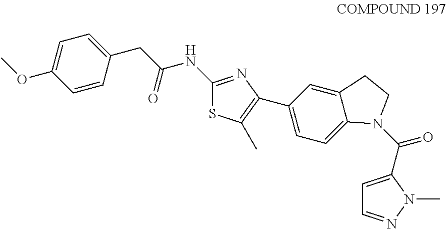

¹H NMR (400 MHz, DMSO-d6) δ 12.28 (s, 1H), 8.14 (s, 1H), 7.59-7.47 (m, 3H), 7.29-7.21 (m, 2H), 6.95-6.86 (m, 2H), 6.79 (s, 1H), 4.24 (t, J=8.3 Hz, 2H), 3.95 (s, 3H), 3.73 (d, J=0.7 Hz, 3H), 3.66 (s, 2H), 3.18 (dd, J=9.0, 7.5 Hz, 2H), 2.45 (s, 3H); Method 1, retention time: 5.551 min; HRMS: m/z (M+H)⁺=488.1743 (Calculated for C₂₆H₂₆N₅O₃S=488.1751).

COMPOUND 208 was prepared according to the method described in Scheme 4 substituting 2-methylcyclopropanecarboxylic acid for 5-(3-nitrophenyl)furan-2-carboxylic acid and substituting N-(4-(indolin-5-yl)-5-methylthiazol-2-yl)-2-(4-methoxyphenyl)acetamide for 5-phenylthiazol-2-amine.

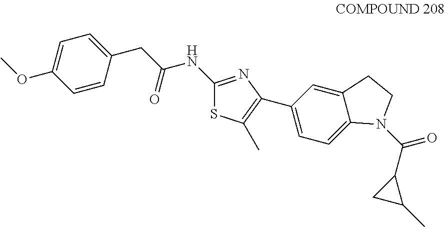 [SMILES: COc1ccc(CC(=O)Nc2nc(-c3ccc4c(c3)CCN4C(=O)C3CC3C)c(C)s2)cc1]

¹H NMR (400 MHz, DMSO-d6) δ 12.24 (s, 1H), 8.05 (d, J=7.8 Hz, 1H), 7.49 (d, J=1.8 Hz, 1H), 7.41 (dd, J=8.4, 1.9 Hz, 1H), 7.24 (d, J=8.6 Hz, 2H), 6.89 (d, J=8.6 Hz, 2H), 4.31 (s, 2H), 3.73 (s, 3H), 3.66 (s, 2H), 3.22 (t, J=8.6 Hz, 2H), 2.43 (s, 3H), 1.71 (s, 1H), 1.34-1.22 (m, 1H), 1.15 (d, J=6.0 Hz, 3H), 1.09 (ddd, J=8.2, 4.6, 3.4 Hz, 1H), 0.71 (dq, J=8.6, 3.8 Hz, 1H); Method 1, retention time: 6.086 min; HRMS: m/z (M+H)⁺=462.1850 (Calculated for C₂₆H₂₈N₃O₃S=462.1846).

COMPOUND 209 was prepared according to the method described in Scheme 4 substituting pivalic acid for 5-(3-nitrophenyl)furan-2-carboxylic acid and substituting (5-(2-amino-5-methylthiazol-4-yl)indolin-1-yl)(o-tolyl)methanone for 5-phenylthiazol-2-amine.

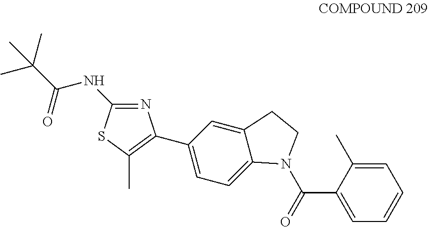 [SMILES: Cc1ccccc1C(=O)N1CCc2cc(-c3nc(NC(=O)C(C)(C)C)sc3C)ccc21]

¹H NMR (400 MHz, DMSO-d6) δ 11.73 (s, 1H), 8.24 (d, J=8.5 Hz, 1H), 7.55 (d, J=1.8 Hz, 2H), 7.33 (dt, J=18.1, 7.0 Hz, 4H), 3.74 (t, J=8.3 Hz, 2H), 3.14 (t, 2H), 2.45 (s, 3H), 2.29 (s, 3H), 1.24 (s, 9H); Method 1, retention time: 6.400 min; HRMS: m/z (M+H)⁺=434.1899 (Calculated for C₂₅H₂₈N₃O₂S=434.1897).

COMPOUND 210 was prepared according to the method described in Scheme 4 substituting 3-phenylpropanoic acid for 5-(3-nitrophenyl)furan-2-carboxylic acid and substituting (5-(2-amino-5-methylthiazol-4-yl)indolin-1-yl)(o-tolyl)methanone for 5-phenylthiazol-2-amine.

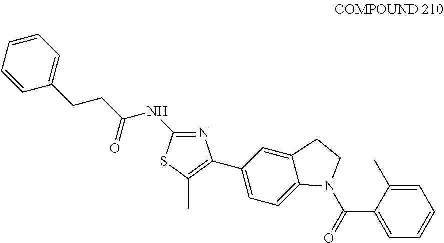 [SMILES: Cc1ccccc1C(=O)N1CCc2cc(-c3nc(NC(=O)CCc4ccccc4)sc3C)ccc21]

¹H NMR (400 MHz, DMSO-d6) δ 12.08 (s, 1H), 8.21 (d, J=8.6 Hz, 1H), 7.50 (d, J=1.7 Hz, 2H), 7.37-7.14 (m, 9H), 3.72 (t, J=8.4 Hz, 2H), 3.15-3.06 (m, 2H), 2.91 (t, J=8.2 Hz, 2H), 2.71 (t, J=7.8 Hz, 2H), 2.44 (s, 3H), 2.27 (s, 3H); Method 1, retention time: 6.503 min; HRMS: m/z (M+H)⁺=482.1904 (Calculated for C₂₉H₂₈N₃O₂S=482.1897).

COMPOUND 233 was prepared according to the method described in Scheme 4 substituting 4-(3-(trifluoromethyl)-3H-diazirin-3-yl)benzoic acid for 5-(3-nitrophenyl)furan-2-carboxylic acid and substituting N-(4-(indolin-5-yl)-5-methylthiazol-2-yl)-2-(4-methoxyphenyl)acetamide for 5-phenylthiazol-2-amine.

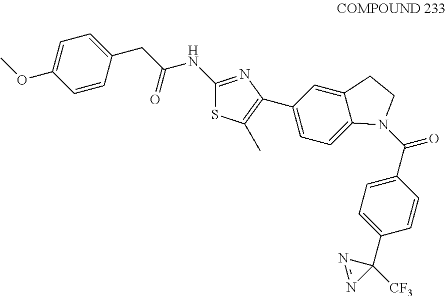 [SMILES: COc1ccc(CC(=O)Nc2nc(-c3ccc4c(c3)CCN4C(=O)c3ccc(C4(C(F)(F)F)N=N4)cc3)c(C)s2)cc1]

¹H NMR (400 MHz, DMSO-d6) δ 12.27 (s, 1H), 7.75 (d, J=8.0 Hz, 2H), 7.54 (d, J=1.7 Hz, 1H), 7.49 (s, 2H), 7.42 (d, J=8.0 Hz, 2H), 7.28-7.22 (m, 2H), 6.93-6.87 (m, 2H), 3.99 (d, J=8.7 Hz, 2H), 3.73 (s, 3H), 3.66 (s, 2H), 3.13 (t, J=8.3 Hz, 2H), 2.45 (d, J=2.9 Hz, 3H); Method 1, retention time: 6.850 min; HRMS: m/z (M+H)⁺=592.1613 (Calculated for C₃₀H₂₅F₃N₅O₃S=592.1625).

COMPOUND 234 was prepared according to the method described in Scheme 4 substituting 2-(3-hydroxyphenyl)acetic acid for 5-(3-nitrophenyl)furan-2-carboxylic acid and substituting (5-(2-amino-5-methylthiazol-4-yl)indolin-1-yl)(o-tolyl)methanone for 5-phenylthiazol-2-amine.

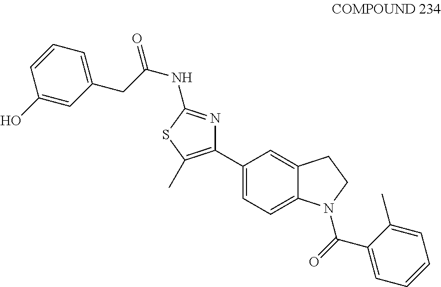 [SMILES: Cc1ccccc1C(=O)N1CCc2cc(-c3nc(NC(=O)Cc4cccc(O)c4)sc3C)ccc21]

¹H NMR (400 MHz, DMSO-d6) δ 12.31 (s, 1H), 9.36 (s, 1H), 8.24 (d, J=8.4 Hz, 1H), 7.53 (d, J=1.7 Hz, 2H), 7.34 (dt, J=18.0, 7.1 Hz, 4H), 7.11 (t, J=7.8 Hz, 1H), 6.74 (d, J=7.5 Hz, 2H), 6.68-6.61 (m, 1H), 3.80-3.69 (m, 2H), 3.64 (s, 2H), 3.21-3.08 (m, 2H), 2.45 (q, J=1.9 Hz, 3H), 2.29 (s, 3H); Method 1, retention time: 5.584 min; HRMS: m/z (M+H)⁺=484.1695 (Calculated for C₂₈H₂₆N₃O₃S=484.1689).

COMPOUND 235 was prepared according to the method described in Scheme 4 substituting 2-(4-hydroxyphenyl)acetic acid for 5-(3-nitrophenyl)furan-2-carboxylic acid and substituting (5-(2-amino-5-methylthiazol-4-yl)indolin-1-yl)(o-tolyl)methanone for 5-phenylthiazol-2-amine.

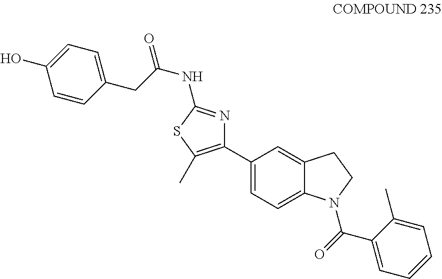 [SMILES: Cc1ccccc1C(=O)N1CCc2cc(-c3nc(NC(=O)Cc4ccc(O)cc4)sc3C)ccc21]

¹H NMR (400 MHz, DMSO-d6) δ 12.26 (s, 1H), 9.29 (s, 1H), 8.24 (d, J=8.5 Hz, 1H), 7.53 (d, J=1.7 Hz, 2H), 7.33 (dt, J=11.4, 7.3 Hz, 4H), 7.12 (d, J=8.2 Hz, 2H), 6.74-6.67 (m, 2H), 3.74 (t, J=8.6 Hz, 2H), 3.60 (s, 2H), 3.14 (q, J=8.4, 7.7 Hz, 2H), 2.45 (q, J=1.8 Hz, 3H), 2.29 (s, 3H); Method 1, retention time: 5.517 min; HRMS: m/z (M+H)⁺=484.1696 (Calculated for C₂₈H₂₆N₃O₃S=484.1689).

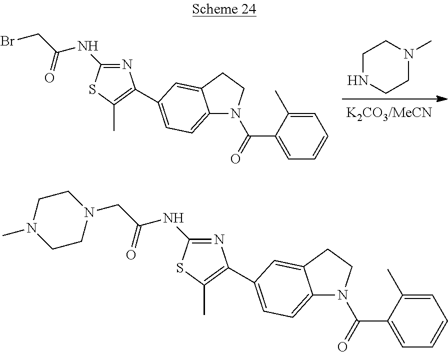

To a the mixture of 2-bromo-N-(5-methyl-4-(1-(2-methylbenzoyl)indolin-5-yl)thiazol-2-yl)acetamide (45 mg, 0.096 mmol) and POTASSIUM CARBONATE (19.83 mg, 0.144 mmol) in MeCN (1 ml) was added 1-methylpiperazine (0.053 ml, 0.478 mmol). The mixture was stirred at r.t. for 3 days. The solvent was removed. The residue was taken up in DMSO and subsequently purified by reverse phase chromatography to afford COMPOUND 72.

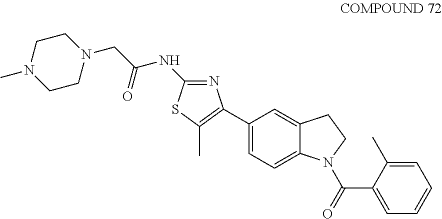 [SMILES: Cc1ccccc1C(=O)N1CCc2cc(-c3nc(NC(=O)CN4CCN(C)CC4)sc3C)ccc21]

¹H NMR (400 MHz, DMSO-d6) δ 11.98 (s, 1H), 8.24 (d, J=8.2 Hz, 1H), 7.56-7.47 (m, 2H), 7.41-7.30 (m, 4H), 3.80-3.69 (m, 2H), 3.46-3.34 (m, 5H), 3.21-2.95 (m, 8H), 2.79 (s, 3H), 2.68-2.57 (m, 2H), 2.29 (s, 3H); Method 1, retention time: 4.434 min; HRMS: m/z (M+H)⁺=490.2285 (Calculated for C₂₇H₃₂N₅O₂S=490.2271).

COMPOUND 227 was prepared according to the method described in Scheme 24 substituting 1-(piperazin-1-yl)ethanone for 1-methylpiperazine.

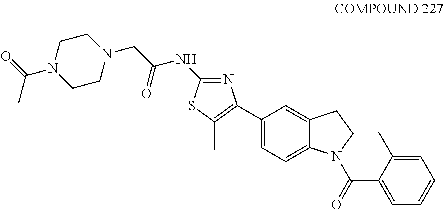 [SMILES: CC(=O)N1CCN(CC(=O)Nc2nc(-c3ccc4c(c3)CCN4C(=O)c3ccccc3C)c(C)s2)CC1]

Method 1, retention time: 4.353 min; HRMS: m/z (M+H)⁺=518.2216 (Calculated for C₂₈H₃₂N₅O₃S=518.2220). ¹H NMR analysis at room temperature is complicated by amide rotamers that are present as a result of the ortho-methyl group in the indolin-1-yl(o-tolyl)methanone segment of the molecule.

COMPOUND 228 was prepared according to the method described in Scheme 24 substituting ethyl piperazine-1-carboxylate for 1-methylpiperazine.

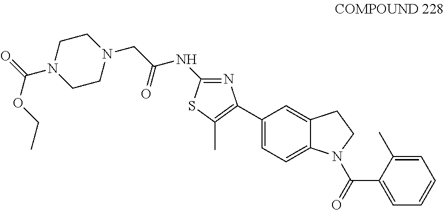 [SMILES: CCOC(=O)N1CCN(CC(=O)Nc2nc(-c3ccc4c(c3)CCN4C(=O)c3ccccc3C)c(C)s2)CC1]

Method 1, retention time: 4.748 min; HRMS: m/z (M+H)⁺=548.2320 (Calculated for C₂₉H₃₄N₅O₄S=548.2326). ¹H NMR analysis at room temperature is complicated by amide rotamers that are present as a result of the ortho-methyl group in the indolin-1-yl(o-tolyl)methanone segment of the molecule.

COMPOUND 229 was prepared according to the method described in Scheme 24 substituting 1-phenylpiperazine for 1-methylpiperazine.

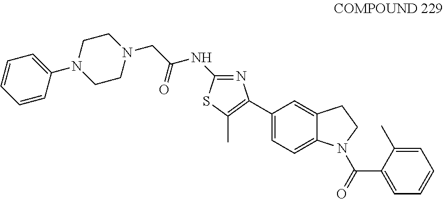 [SMILES: Cc1ccccc1C(=O)N1CCc2cc(-c3nc(NC(=O)CN4CCN(c5ccccc5)CC4)sc3C)ccc21]

Method 1, retention time: 5.071 min; HRMS: m/z (M+H)⁺=552.2407 (Calculated for C₃₂H₃₄N₅O₂S=552.2428). ¹H NMR analysis at room temperature is complicated by amide rotamers that are present as a result of the ortho-methyl group in the indolin-1-yl(o-tolyl)methanone segment of the molecule.

COMPOUND 212 was prepared according to the method described in Scheme 24 substituting cyclopropanecarbonyl chloride for 2-ethylbenzoyl chloride and substituting N-(5-methyl-4-(1-(2-methylbenzoyl)indolin-5-yl)thiazol-2-yl)-2-(piperazin-1-yl)acetamide for 2-(benzo[d][1,3]dioxol-5-yl)-N-(4-(indolin-5-yl)-5-methylthiazol-2-yl)acetamide.

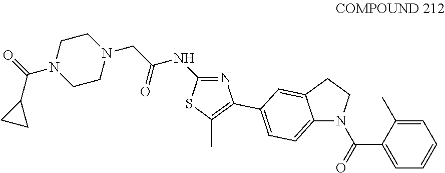 [SMILES: Cc1ccccc1C(=O)N1CCc2cc(-c3nc(NC(=O)CN4CCN(C(=O)C5CC5)CC4)sc3C)ccc21]

Method 1, retention time: 4.544 min; HRMS: m/z (M+2H)²=273.1243 (Calculated for C₃₀H₃₅N₅O₃S=273.124). ¹H NMR analysis at room temperature is complicated by amide rotamers that are present as a result of the ortho-methyl group in the indolin-1-yl(o-tolyl)methanone segment of the molecule.

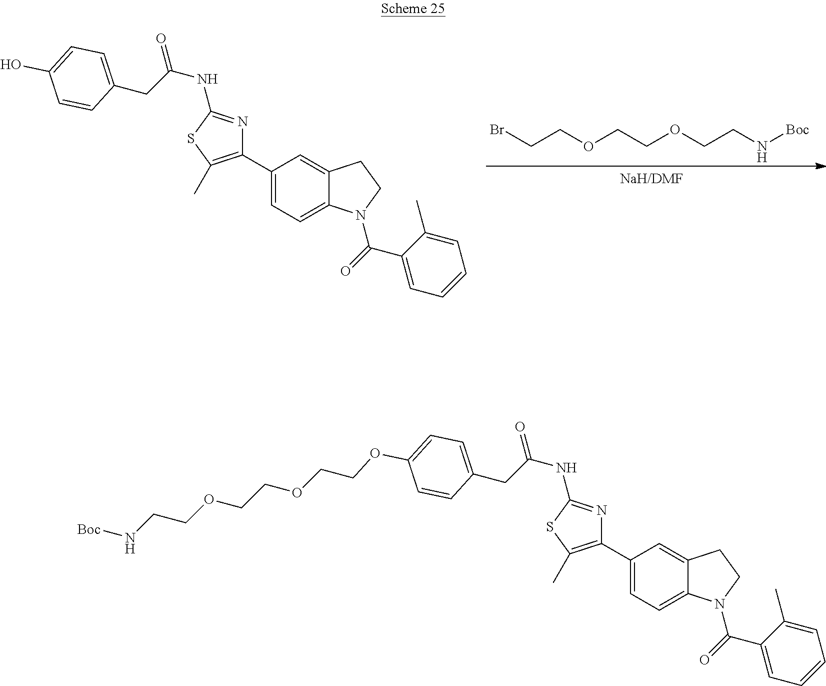 [SMILES: Cc1ccccc1C(=O)N1CCc2cc(-c3nc(NC(=O)Cc4ccc(O)cc4)sc3C)ccc21 ; CC(C)(C)OC(=O)NCCOCCOCCBr ; Cc1ccccc1C(=O)N1CCc2cc(-c3nc(NC(=O)Cc4ccc(OCCOCCOCCNC(=O)OC(C)(C)C)cc4)sc3C)ccc21]

To the solution of 2-(4-hydroxyphenyl)-N-(5-methyl-4-(1-(2-methylbenzoyl)indolin-5-yl)thiazol-2-yl)acetamide (0.1 g, 0.207 mmol) in DMF (1.5 ml) was added NaH (0.025 g, 0.620 mmol). The mixture was stirred at r.t. for 30 mins. tert-butyl (2-(2-(2-bromoethoxy)ethoxy)ethyl)carbamate (0.071 g, 0.227 mmol) was added to the reaction mixture. The reaction mixture was stirred at r.t. for 5 h. H₂O was added to the mixture and extracted with EtOAc (2×). The organic layer was washed with Sat. Na₂SO₄ (3×) and brine and dried over Na₂SO₄, and concentrated. The residue was taken up in DMSO and subsequently purified by reverse phase chromatography to afford COMPOUND 236.

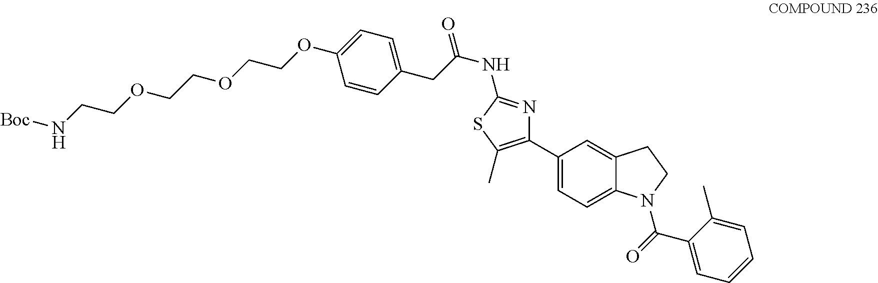 [SMILES: Cc1ccccc1C(=O)N1CCc2cc(-c3nc(NC(=O)Cc4ccc(OCCOCCOCCNC(=O)OC(C)(C)C)cc4)sc3C)ccc21]

¹H NMR (400 MHz, DMSO-d6) δ 12.29 (s, 1H), 8.24 (d, J=8.4 Hz, 1H), 7.53 (d, J=1.8 Hz, 2H), 7.39-7.29 (m, 4H), 7.24 (d, J=8.2 Hz, 2H), 6.90 (d, J=8.2 Hz, 2H), 6.74 (t, J=5.7 Hz, 1H), 4.06 (dd, J=5.8, 3.6 Hz, 2H), 3.78-3.69 (m, 4H), 3.66 (s, 2H), 3.60-3.54 (m, 2H), 3.54-3.48 (m, 2H), 3.38 (t, J=6.2 Hz, 2H), 3.13 (t, J=8.4 Hz, 2H), 3.06 (q, J=6.0 Hz, 2H), 2.45 (dq, J=4.0, 1.8 Hz, 3H), 2.29 (s, 3H), 1.36 (s, 9H); Method 1, retention time: 6.421 min; HRMS: m/z (M+H)⁺=715.3161 (Calculated for C₃₉H₄₇N₄O₇S=715.3160).

COMPOUND 237 was prepared according to the method described in Scheme 25 substituting 2-(3-hydroxyphenyl)-N-(5-methyl-4-(1-(2-methylbenzoyl)indolin-5-yl)thiazol-2-yl)acetamide for 2-(4-hydroxyphenyl)-N-(5-methyl-4-(1-(2-methylbenzoyl)indolin-5-yl)thiazol-2-yl)acetamide.

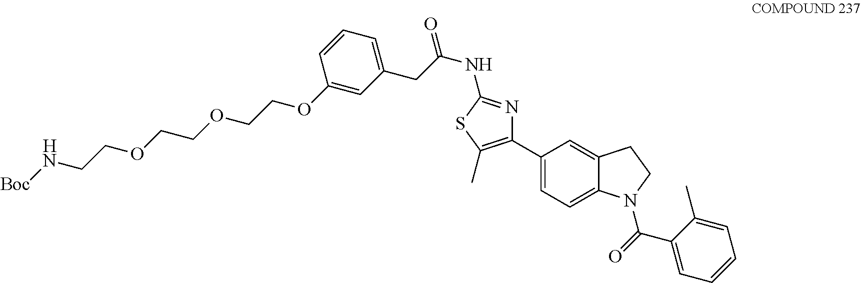 [SMILES: Cc1ccccc1C(=O)N1CCc2cc(-c3nc(NC(=O)Cc4cccc(OCCOCCOCCNC(=O)OC(C)(C)C)c4)sc3C)ccc21]

¹H NMR (400 MHz, DMSO-d6) δ 12.32 (s, 1H), 8.24 (d, J=8.6 Hz, 1H), 7.53 (d, J=1.8 Hz, 2H), 7.33 (dt, J=18.1, 7.0 Hz, 4H), 7.23 (t, J=7.9 Hz, 1H), 6.94-6.87 (m, 2H), 6.84 (dd, J=8.2, 2.6 Hz, 1H), 6.78-6.72 (m, 1H), 4.07 (dd, J=5.8, 3.5 Hz, 2H), 3.73 (dd, J=11.4, 6.5 Hz, 6H), 3.62-3.54 (m, 2H), 3.52 (dt, J=6.4, 2.8 Hz, 2H), 3.42-3.34 (m, 2H), 3.18-3.09 (m, 2H), 3.05 (q, J=6.0 Hz, 2H), 2.48-2.41 (m, 3H), 2.29 (s, 3H), 1.36 (s, 9H); Method 1, retention time: 6.492 min; HRMS: m/z (M+H)⁺=715.3126 (Calculated for C₃₀H₃₇N₅O₃S=715.3160).

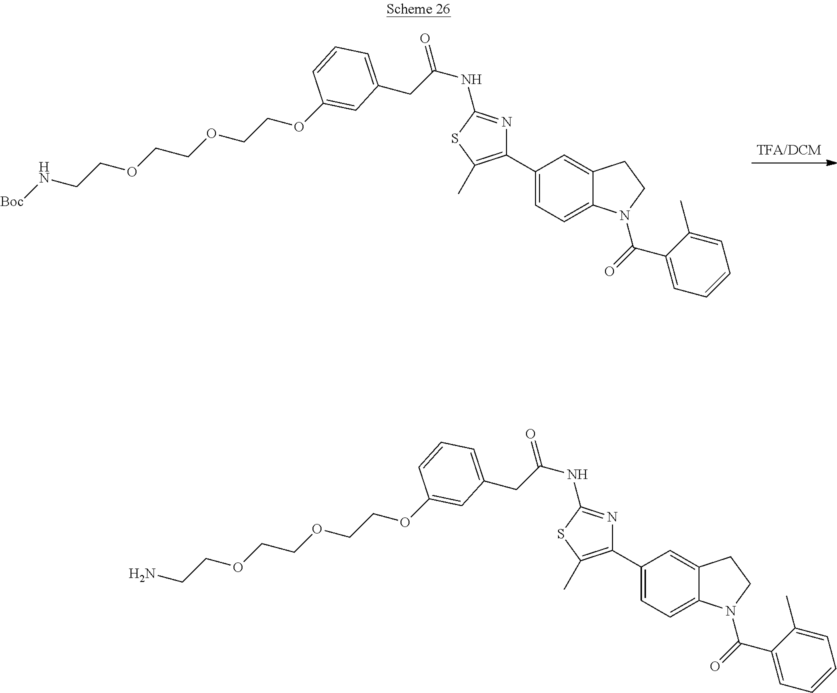 [SMILES: Cc1ccccc1C(=O)N1CCc2cc(-c3nc(NC(=O)Cc4cccc(OCCOCCOCCNC(=O)OC(C)(C)C)c4)sc3C)ccc21 ; Cc1ccccc1C(=O)N1CCc2cc(-c3nc(NC(=O)Cc4cccc(OCCOCCOCCN)c4)sc3C)ccc21]

To a solution of tert-butyl (2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl)carbamate (0.15 g, 0.210 mmol) in DCM (2 ml) was added TFA (0.2 ml, 2.60 mmol). The mixture was stirred at r.t. for 2 hrs. The solvent was removed. The residue was taken up in DMSO and subsequently purified by reverse phase chromatography to afford COMPOUND 238.

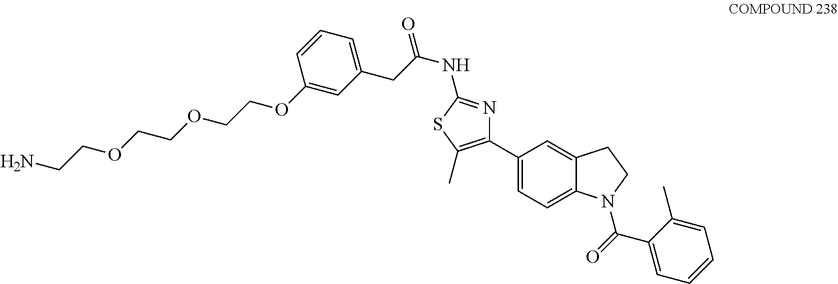 [SMILES: Cc1ccccc1C(=O)N1CCc2cc(-c3nc(NC(=O)Cc4cccc(OCCOCCOCCN)c4)sc3C)ccc21]

Method 1, retention time: 4.839 min; HRMS: m/z (M+H)⁺=615.2645 (Calculated for C₃₄H₃₉N₄O₅S=615.2636). ¹H NMR analysis at room temperature is complicated by amide rotamers that are present as a result of the ortho-methyl group in the indolin-1-yl(o-tolyl)methanone segment of the molecule.

COMPOUND 239 was prepared according to the method described in Scheme 26 substituting tert-butyl (2-(2-(2-(4-(2-((5-methyl-4-(1-(2-methylbenzoyl)indolin-5-yl)thiazol-2-yl)amino)-2-oxoethyl)phenoxy)ethoxy)ethoxy)ethyl)carbamate for tert-butyl (2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl)carbamate.

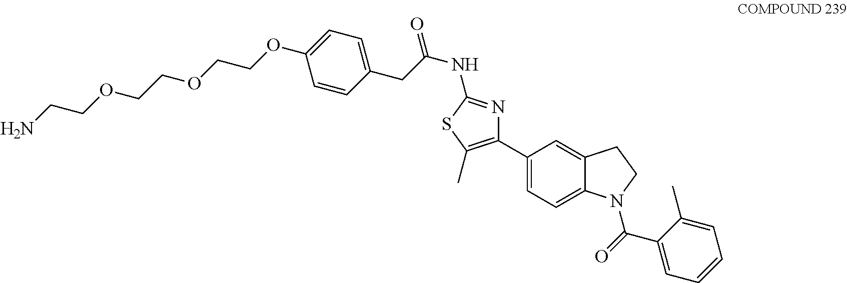 [SMILES: Cc1ccccc1C(=O)N1CCc2cc(-c3nc(NC(=O)Cc4ccc(OCCOCCOCCN)cc4)sc3C)ccc21]

Method 1, retention time: 4.814 min; HRMS: m/z (M+H)⁺=615.2640 (Calculated for C₃₄H₃₉N₄O₅S=615.2636). ¹H NMR analysis at room temperature is complicated by amide rotamers that are present as a result of the ortho-methyl group in the indolin-1-yl(o-tolyl)methanone segment of the molecule.

COMPOUND 240 was prepared according to the method described in Scheme 4 substituting 5-((3 aS,4S,6aR)-2-oxohexahydro-1H-thieno[3,4-d]imidazol-4-yl)pentanoic acid for 5-(3-nitrophenyl)furan-2-carboxylic acid and substituting 2-(3-(2-(2-(2-aminoethoxy)ethoxy)ethoxy)phenyl)-N-(5-methyl-4-(1-(2-methylbenzoyl)indolin-5-yl)thiazol-2-yl)acetamide for 5-phenylthiazol-2-amine.

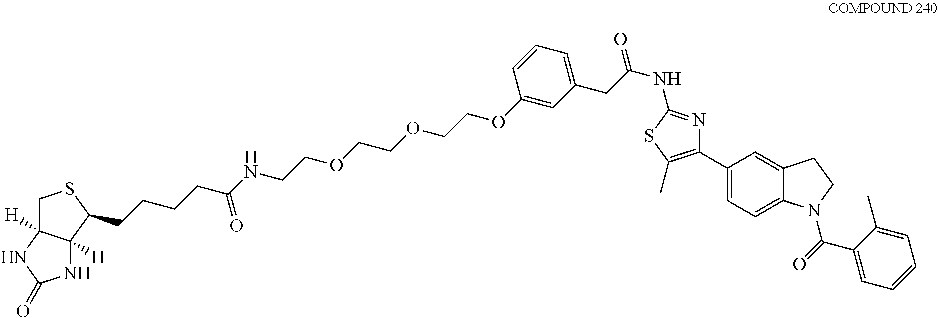 [SMILES: Cc1ccccc1C(=O)N1CCc2cc(-c3nc(NC(=O)Cc4cccc(OCCOCCOCCNC(=O)CCCC[C@@H]5SC[C@@H]6NC(=O)N[C@@H]65)c4)sc3C)ccc21]

Method 1, retention time: 5.491 min; HRMS: m/z (M+H)⁺=842.3435 (Calculated for C₄₄H₅₃N₆O₇S₂=842.3442). ¹H NMR analysis at room temperature is complicated by amide rotamers that are present as a result of the ortho-methyl group in the indolin-1-yl(o-tolyl)methanone segment of the molecule.

COMPOUND 241 was prepared according to the method described in Scheme 4 substituting 5-((3 aS,4S,6aR)-2-oxohexahydro-1H-thieno[3,4-d]imidazol-4-yl)pentanoic acid for 5-(3-nitrophenyl)furan-2-carboxylic acid and substituting 2-(4-(2-(2-(2-aminoethoxy)ethoxy)ethoxy)phenyl)-N-(5-methyl-4-(1-(2-methylbenzoyl)indolin-5-yl)thiazol-2-yl)acetamide for 5-phenylthiazol-2-amine.

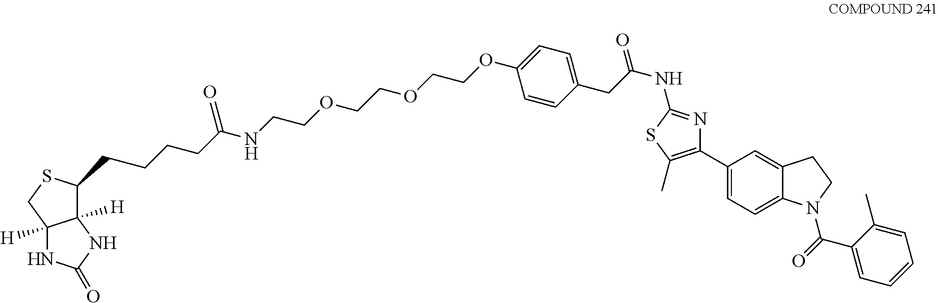 [SMILES: Cc1ccccc1C(=O)N1CCc2cc(-c3nc(NC(=O)Cc4ccc(OCCOCCOCCNC(=O)CCCC[C@@H]5SC[C@@H]6NC(=O)N[C@@H]65)cc4)sc3C)ccc21]

Method 1, retention time: 5.431 min; HRMS: m/z (M+H)⁺=841.3387 (Calculated for C₄₄H₅₃N₆O₇S₂=841.3412). ¹H NMR analysis at room temperature is complicated by amide rotamers that are present as a result of the ortho-methyl group in the indolin-1-yl(o-tolyl)methanone segment of the molecule.

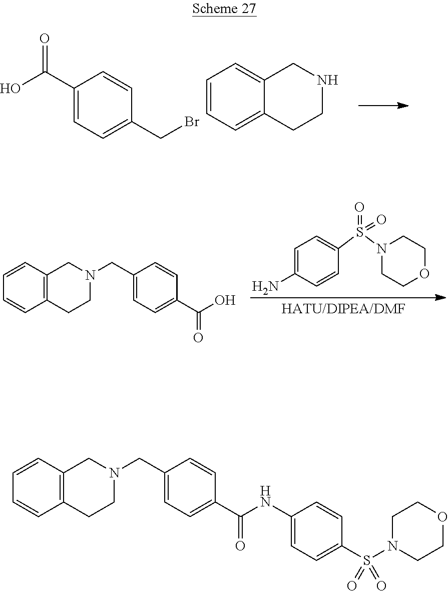

### Scheme 27, Step 1 Compound 90.

To a solution of 4-(bromomethyl)benzoic acid (1 g, 4.65 mmol) in THF (Volume: 20 ml) was added 1,2,3,4-tetrahydroisoquinoline (0.885 ml, 6.98 mmol) which resulted in the precipitation of a solid. The reaction mixture was refluxed for 4 hrs. The solvent was removed under vacuo. The resultant solid was dissolved in 1N NaOH and extracted with DCM. The aqueous layer was acidified to pH=1 using concentrated HCl resulting in precipitation of the final product. LCMS: m/z (M+H)+=268.0. ¹H NMR (400 MHz, DMSO-d6) δ 13.15 (s, 1H), 10.20 (s, 1H), 8.03 (d, J=8.9 Hz, 1H), 7.67 (d, J=7.8 Hz, 2H), 7.21 (d, J=19.9 Hz, 4H), 4.54 (s, 2H), 4.34 (s, 2H), 3.67 (s, 1H), 3.08 (s, 3H).

Scheme 27, Step 2 COMPOUND 48 was prepared according to the method described in Scheme 4 substituting 4-(morpholinosulfonyl)aniline for 5-phenylthiazol-2-amine and substituting 4-((3,4-dihydroisoquinolin-2(1H)-yl)methyl)benzoic acid for 5-(3-nitrophenyl)furan-2-carboxylic acid.

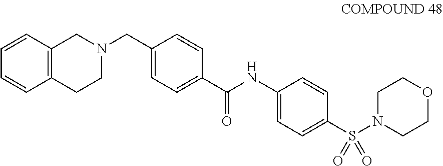 [SMILES: O=C(Nc1ccc(S(=O)(=O)N2CCOCC2)cc1)c1ccc(CN2CCc3ccccc3C2)cc1]

¹H NMR (400 MHz, DMSO-d6) δ 10.75 (s, 1H), 8.18-8.00 (m, 4H), 7.85-7.66 (m, 4H), 7.25 (d, J=20.9 Hz, 4H), 4.58 (s, 2H), 4.39 (s, 2H), 3.77-3.58 (m, 5H), 3.11 (d, J=6.7 Hz, 2H), 2.92-2.82 (m, 4H), 2.54 (s, 1H); Method 1, retention time: 4.036 min; HRMS: m/z (M+H)⁺=492.1954 (Calculated for C₂₇H₃₀N₃O₄S=492.1952).

COMPOUND 211 was prepared according to the method described in Scheme 27 substituting 3-((3,4-dihydroisoquinolin-2(1H)-yl)methyl)benzoic acid for 4-((3,4-dihydroisoquinolin-2(1H)-yl)methyl)benzoic acid.

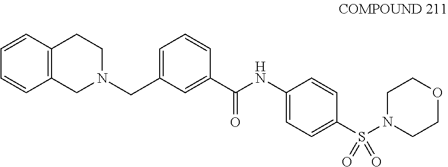 [SMILES: O=C(Nc1ccc(S(=O)(=O)N2CCOCC2)cc1)c1cccc(CN2CCc3ccccc3C2)c1]

¹H NMR (400 MHz, DMSO-d6) δ 10.77 (s, 1H), 8.19-8.09 (m, 2H), 8.09-8.04 (m, 2H), 7.81 (d, J=7.6 Hz, 1H), 7.79-7.73 (m, 2H), 7.73-7.67 (m, 1H), 7.33-7.18 (m, 4H), 4.58 (d, J=6.3 Hz, 2H), 4.39 (d, J=13.4 Hz, 2H), 3.69 (d, J=14.5 Hz, 2H), 3.67-3.60 (m, 4H), 3.11 (s, 2H), 2.90-2.82 (m, 4H); Method 1, retention time: 4.078 min; HRMS: m/z (M+H)⁺=492.1969 (Calculated for C₂₇H₃₀N₃O₄S=492.1952).

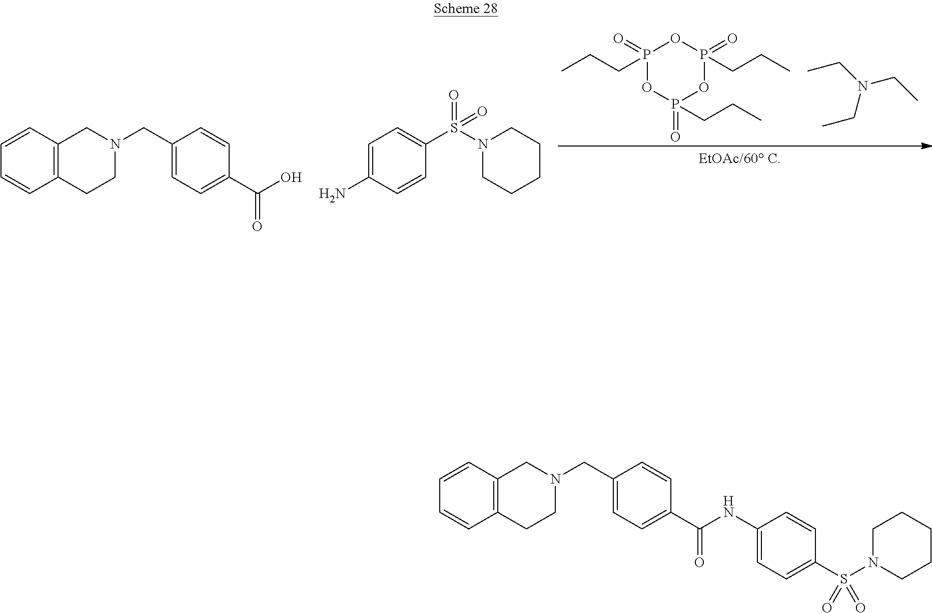

The mixture of 4-((3,4-dihydroisoquinolin-2(1H)-yl)methyl)benzoic acid (0.1 g, 0.374 mmol), 4-(piperidin-1-ylsulfonyl)aniline (0.082 g, 0.340 mmol), 2,4,6-tripropyl-1,3,5,2,4,6-trioxatriphosphinane 2,4,6-trioxide (0.347 ml, 0.680 mmol)(i50% in EtOAc) and TRIETHYLAMINE (0.237 ml, 1.700 mmol) in EtOAc (1 ml) was stirred at 60° C. for 30 min. The solvent was removed. The crude product was purified by reverse phase chromatography to afford COMPOUND 38.

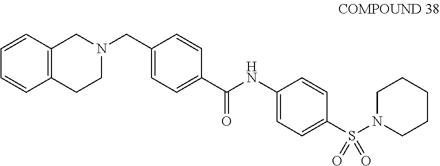 [SMILES: O=C(Nc1ccc(S(=O)(=O)N2CCCCC2)cc1)c1ccc(CN2CCc3ccccc3C2)cc1]

¹H NMR (400 MHz, DMSO-d6) δ 10.71 (s, 1H), 8.15-7.98 (m, 4H), 7.82-7.63 (m, 4H), 7.36-7.13 (m, 4H), 4.58 (s, 2H), 4.39 (s, 2H), 3.70 (d, J=11.9 Hz, 2H), 3.14 (d, J=19.6 Hz, 2H), 2.94-2.84 (m, 4H), 1.55 (p, J=5.6 Hz, 4H), 1.37 (q, J=5.9, 5.4 Hz, 2H); Method 1, retention time: 4.589 min; HRMS: m/z (M+H)⁺=490.2139 (Calculated for C₂₈H₃₂N₃O₃S=490.2159).

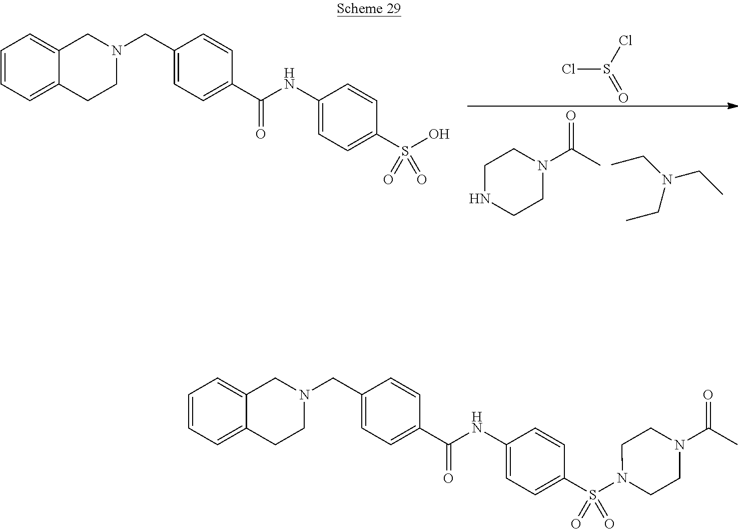

To a suspension of 4-(4-((3,4-dihydroisoquinolin-2(1H)-yl)methyl)benzamido)benzenesulfonic acid (50 mg, 0.118 mmol) in SOCl₂ (0.4 ml, 5.48 mmol) was added a drop DMF. The mixture was stirred at 60° C. for 1 hr, and diluted with DCM, and concentrated. To the residue were added DCM (1 ml), TRIETHYLAMINE (0.247 ml, 1.775 mmol) and 1-(piperazin-1-yl)ethanone (45.5 mg, 0.355 mmol). The mixture was stirred at r.t. for 3 hrs. The solvent was removed. The crude product was purified by reverse phase chromatography to afford COMPOUND 198.

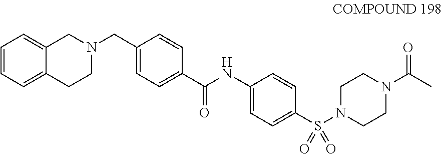 [SMILES: CC(=O)N1CCN(S(=O)(=O)c2ccc(NC(=O)c3ccc(CN4CCc5ccccc5C4)cc3)cc2)CC1]

¹H NMR (400 MHz, DMSO-d6) δ 10.75 (s, 1H), 8.19-7.98 (m, 4H), 7.75 (dq, J=8.9, 2.1 Hz, 4H), 7.35-7.16 (m, 4H), 4.58 (s, 2H), 4.39 (s, 2H), 3.68 (s, 2H), 3.51 (q, J=5.2 Hz, 4H), 3.11 (s, 2H), 2.91 (t, J=5.0 Hz, 2H), 2.85 (t, J=5.2 Hz, 2H), 1.94 (s, 3H); Method 1, retention time: 3.779 min; HRMS: m/z (M+H)⁺=533.2197 (Calculated for C₂₉H₃₃N₄O₄S=533.2217).

COMPOUND 199 was prepared according to the method described in Scheme 29 substituting ethyl piperazine-1-carboxylate for 1-(piperazin-1-yl)ethanone.

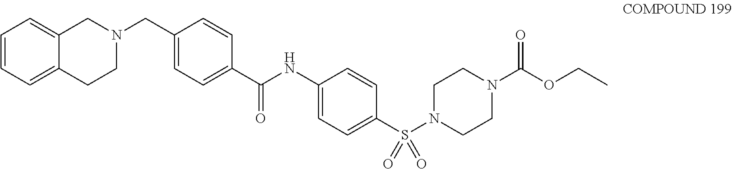 [SMILES: CCOC(=O)N1CCN(S(=O)(=O)c2ccc(NC(=O)c3ccc(CN4CCc5ccccc5C4)cc3)cc2)CC1]

¹H NMR (400 MHz, DMSO-d6) δ 10.75 (s, 1H), 8.20-7.99 (m, 4H), 7.82-7.63 (m, 4H), 7.36-7.18 (m, 4H), 4.58 (s, 2H), 4.39 (s, 2H), 3.99 (q, J=7.1 Hz, 2H), 3.69 (d, J=12.4 Hz, 1H), 3.45 (t, J=5.0 Hz, 5H), 3.12 (s, 2H), 2.88 (t, J=5.0 Hz, 4H), 1.13 (t, J=7.1 Hz, 3H); Method 1, retention time: 4.364 min; HRMS: m/z (M+H)⁺=563.2342 (Calculated for C₃₀H₃₅N₄O₅S=563.2323).

COMPOUND 200 was prepared according to the method described in Scheme 29 substituting 1-phenylpiperazine for 1-(piperazin-1-yl)ethanone.

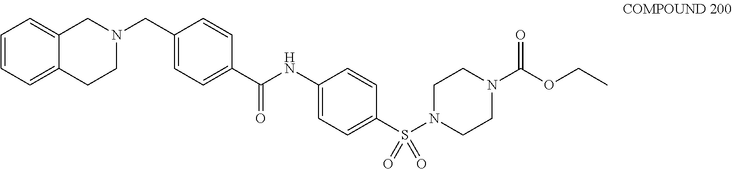 [SMILES: CCOC(=O)N1CCN(S(=O)(=O)c2ccc(NC(=O)c3ccc(CN4CCc5ccccc5C4)cc3)cc2)CC1]

¹H NMR (400 MHz, DMSO-d6) δ 10.73 (s, 1H), 8.16-7.95 (m, 4H), 7.75 (dd, J=21.5, 8.3 Hz, 4H), 7.36-7.11 (m, 6H), 6.95-6.82 (m, 2H), 6.78 (tt, J=7.3, 1.1 Hz, 1H), 4.56 (d, J=4.9 Hz, 2H), 4.36 (s, 2H), 3.48 (s, 4H), 3.25-3.11 (m, 4H), 3.00 (t, J=5.0 Hz, 4H); Method 1, retention time: 4.958 min; HRMS: m/z (M+2H)²=284.1237 (Calculated for C₃₃H₃₆N₄O₃S=284.1249).

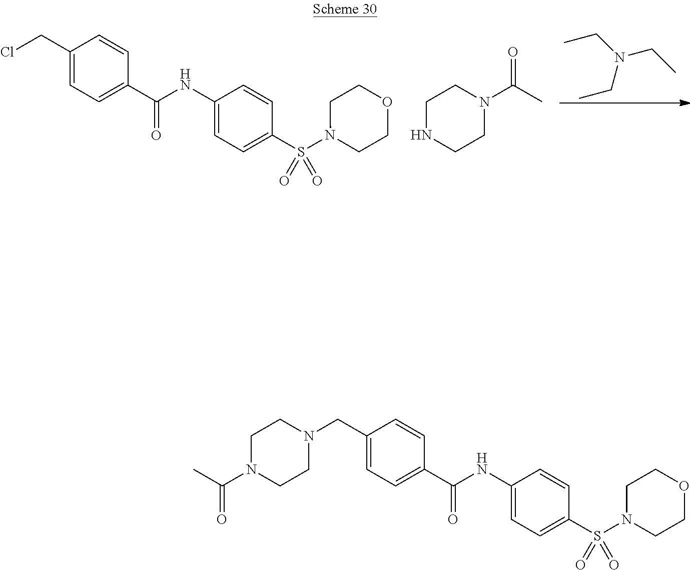

To a solution of 4-(chloromethyl)-N-(4-(morpholinosulfonyl)phenyl)benzamide (50 mg, 0.127 mmol) in THF (1 ml) were added TRIETHYLAMINE (0.088 ml, 0.633 mmol) and 1-(piperazin-1-yl)ethanone (16.23 mg, 0.127 mmol). The mixture was stirred at r.t. for 3 days. The solvent was removed. The residue was dissolved in DMSO. The crude product was purified by reverse phase chromatography to afford COMPOUND 201.

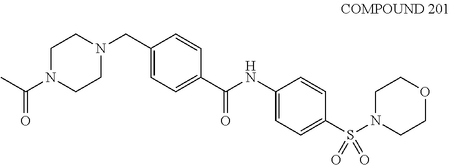 [SMILES: CC(=O)N1CCN(Cc2ccc(C(=O)Nc3ccc(S(=O)(=O)N4CCOCC4)cc3)cc2)CC1]

¹H NMR (400 MHz, DMSO-d6) δ 10.73 (s, 1H), 8.08 (d, J=8.5 Hz, 4H), 7.75 (d, J=8.4 Hz, 2H), 7.67 (s, 2H), 4.45 (s, 2H), 3.69-3.55 (m, 4H), 3.38 (s, 8H), 2.86 (t, J=4.6 Hz, 4H), 2.03 (s, 3H); Method 1, retention time: 3.386 min; HRMS: m/z (M+H)⁺=487.2016 (Calculated for C₂₄H₃₁N₄O₅S=487.2010).

COMPOUND 202 was prepared according to the method described in Scheme 30 substituting 1-phenylpiperazine for 1-(piperazin-1-yl)ethanone.

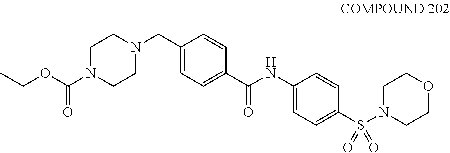 [SMILES: CCOC(=O)N1CCN(Cc2ccc(C(=O)Nc3ccc(S(=O)(=O)N4CCOCC4)cc3)cc2)CC1]

¹H NMR (400 MHz, DMSO-d6) δ 10.73 (s, 1H), 8.08 (d, J=8.8 Hz, 2H), 7.75 (d, J=8.8 Hz, 2H), 7.68 (d, J=24.5 Hz, 4H), 4.44 (s, 2H), 4.07 (q, J=7.1 Hz, 2H), 3.64 (dd, J=5.9, 3.6 Hz, 4H), 3.26 (d, J=70.5 Hz, 8H), 2.91-2.83 (m, 4H), 1.19 (t, J=7.1 Hz, 3H); Method 1, retention time: 3.831 min; HRMS: m/z (M+H)⁺=517.2129 (Calculated for C₂₅H₃₃N₄O₆S=517.2115).

COMPOUND 203 was prepared according to the method described in Scheme 30 substituting 1-phenylpiperazine for 1-(piperazin-1-yl)ethanone.

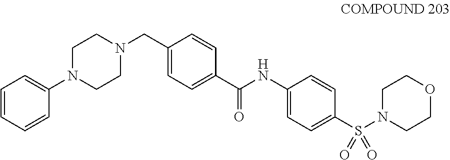 [SMILES: O=C(Nc1ccc(S(=O)(=O)N2CCOCC2)cc1)c1ccc(CN2CCN(c3ccccc3)CC2)cc1]

¹H NMR (400 MHz, DMSO-d6) δ 10.75 (s, 1H), 8.21-7.98 (m, 4H), 7.76 (d, J=8.6 Hz, 4H), 7.26 (t, J=7.6 Hz, 2H), 6.99 (d, J=8.1 Hz, 2H), 6.87 (s, 1H), 4.52 (s, 2H), 3.85 (d, J=13.2 Hz, 2H), 3.64 (t, J=4.6 Hz, 4H), 3.29-3.15 (m, 4H), 3.01 (d, J=13.0 Hz, 2H), 2.87 (t, J=4.7 Hz, 4H); Method 1, retention time: 4.168 min; HRMS: m/z (M+H)⁺=521.2207 (Calculated for C₂₈H₃₃N₄O₄S=521.2217).

COMPOUND 204 was prepared according to the method described in Scheme 30 substituting isoindoline for 1-(piperazin-1-yl)ethanone.

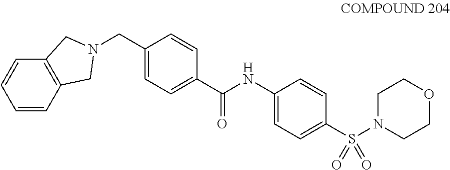 [SMILES: O=C(Nc1ccc(S(=O)(=O)N2CCOCC2)cc1)c1ccc(CN2Cc3ccccc3C2)cc1]

¹H NMR (400 MHz, DMSO-d6) δ 10.75 (s, 1H), 8.13-8.06 (m, 4H), 7.76 (d, J=8.9 Hz, 2H), 7.40 (dt, J=13.0, 5.0 Hz, 6H), 4.93-4.59 (m, 6H), 3.68-3.59 (m, 4H), 2.92-2.83 (m, 4H); Method 1, retention time: 3.904 min; HRMS: m/z (M+H)=478.1782 (Calculated for C₂₆H₂₈N₃O₄S=478.1795).

COMPOUND 205 was prepared according to the method described in Scheme 30 substituting 4-phenylpiperidine for 1-(piperazin-1-yl)ethanone.

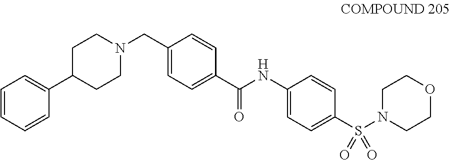 [SMILES: O=C(Nc1ccc(S(=O)(=O)N2CCOCC2)cc1)c1ccc(CN2CCC(c3ccccc3)CC2)cc1]

¹H NMR (400 MHz, DMSO-d6) δ 10.75 (s, 1H), 8.09 (dd, J=8.8, 2.4 Hz, 4H), 7.83-7.68 (m, 4H), 7.39-7.30 (m, 2H), 7.30-7.18 (m, 3H), 4.46 (d, J=4.9 Hz, 2H), 3.64 (dd, J=5.9, 3.6 Hz, 4H), 3.49 (d, J=12.2 Hz, 2H), 3.12 (q, J=12.0, 11.0 Hz, 2H), 2.90-2.75 (m, 5H), 2.02 (d, J=14.1 Hz, 2H), 1.87 (q, J=12.4, 11.7 Hz, 2H); Method 1, retention time: 4.328 min; HRMS: m/z (M+H)⁺=520.2260 (Calculated for C₂₉H₃₄N₃O₄S=520.2265).

COMPOUND 206 was prepared according to the method described in Scheme 30 substituting 3-methyl-1,2,3,4-tetrahydroisoquinoline for 1-(piperazin-1-yl)ethanone.

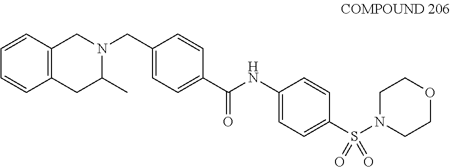 [SMILES: CC1Cc2ccccc2CN1Cc1ccc(C(=O)Nc2ccc(S(=O)(=O)N3CCOCC3)cc2)cc1]

¹H NMR (400 MHz, DMSO-d6) δ 10.74 (s, 1H), 8.07 (d, J=8.9 Hz, 2H), 7.78-7.67 (m, 4H), 7.37-7.13 (m, 6H), 4.65 (dd, J=66.2, 13.5 Hz, 1H), 4.35 (ddd, J=25.3, 13.6, 7.3 Hz, 3H), 4.02-3.83 (m, 1H), 3.66-3.56 (m, 4H), 3.01-2.78 (m, 6H), 1.41 (dd, J=28.9, 6.6 Hz, 3H); Method 1, retention time: 4.119 min; HRMS: m/z (M+H)⁺=506.2121 (Calculated for C₂₈H₃₂N₃O₄S=506.2108).

COMPOUND 207 was prepared according to the method described in Scheme 30 substituting 3-methyl-1,2,3,4-tetrahydroisoquinoline for 1-(piperazin-1-yl)ethanone.

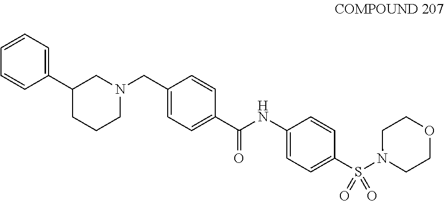 [SMILES: O=C(Nc1ccc(S(=O)(=O)N2CCOCC2)cc1)c1ccc(CN2CCCC(c3ccccc3)C2)cc1]

¹H NMR (400 MHz, DMSO-d6) δ 10.73 (s, 1H), 8.09-8.02 (m, 4H), 7.78-7.72 (m, 2H), 7.72-7.67 (m, 2H), 7.36 (q, J=6.1, 4.7 Hz, 3H), 7.31-7.24 (m, 3H), 4.47-4.39 (m, 2H), 3.63 (dd, J=5.9, 3.5 Hz, 4H), 3.40 (d, J=20.9 Hz, 8H), 2.90-2.83 (m, 4H); Method 1, retention time: 4.338 min; HRMS: m/z (M+H)⁺=520.2248 (Calculated for C₂₉H₃₄N₃O₄S=520.2265).

COMPOUND 230 was prepared according to the method described in Scheme 30 substituting 1-methyl-1,2,3,4-tetrahydroisoquinoline for 1-(piperazin-1-yl)ethanone.

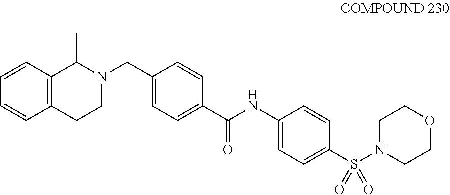 [SMILES: CC1c2ccccc2CCN1Cc1ccc(C(=O)Nc2ccc(S(=O)(=O)N3CCOCC3)cc2)cc1]

¹H NMR (400 MHz, DMSO-d6) δ 10.75 (s, 1H), 8.15-8.03 (m, 4H), 7.75 (dd, J=8.1, 6.1 Hz, 4H), 7.29 (qd, J=11.5, 10.2, 6.6 Hz, 4H), 4.60 (d, J=12.0 Hz, 2H), 4.55-4.34 (m, 1H), 3.64 (dd, J=5.9, 3.5 Hz, 4H), 3.41 (s, 3H), 3.10 (s, 1H), 2.91-2.82 (m, 4H), 1.65 (dd, J=15.8, 6.8 Hz, 3H); Method 1, retention time: 4.159 min; HRMS: m/z (M+H)⁺=506.2092 (Calculated for C₂₈H₃₂N₃O₄S=506.2108).

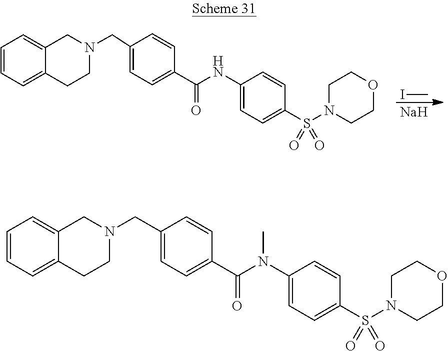

To a solution of 4-((3,4-dihydroisoquinolin-2(1H)-yl)methyl)-N-(4-(morpholinosulfonyl)phenyl)benzamide (60 mg, 0.122 mmol) in DMF (1.5 ml) was added NaH (14.64 mg, 0.610 mmol). The mixture was stirred at r.t. for 10 min. To the mixture was added IODOMETHANE (0.038 ml, 0.610 mmol). The reaction mixture was stirred at r.t. for 30 min. Water was carefully added. The crude product was purified by reverse phase chromatography to afford COMPOUND 225.

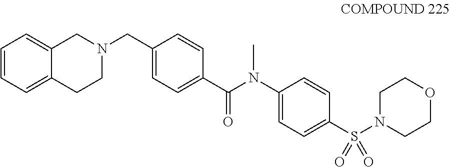 [SMILES: CN(C(=O)c1ccc(CN2CCc3ccccc3C2)cc1)c1ccc(S(=O)(=O)N2CCOCC2)cc1]

¹H NMR (400 MHz, DMSO-d6) δ 7.62 (d, J=8.4 Hz, 2H), 7.53-7.39 (m, 6H), 7.37-7.14 (m, 4H), 4.57 (dt, J=9.4, 5.6 Hz, 2H), 3.67 (q, J=7.5, 6.6 Hz, 2H), 3.56 (dd, J=8.9, 4.4 Hz, 4H), 3.46 (s, 3H), 3.25-3.14 (m, 2H), 2.87 (s, 2H), 2.83-2.72 (m, 4H); Method 1, retention time: 3.955 min; HRMS: m/z (M+H)⁺=506.2088 (Calculated for C₂₈H₃₂N₃O₄S=506.2108).

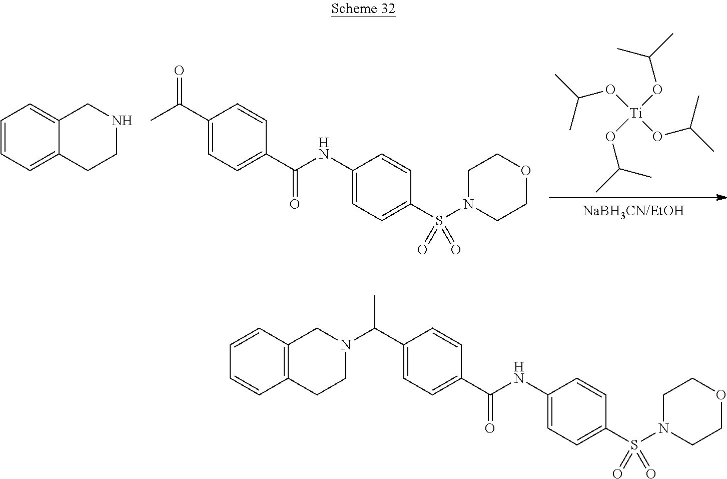

A mixture of Reactant 1 (0.14 g, 0.360 mmol), TITANIUM(IV) ISOPROPOXIDE (0.176 ml, 0.601 mmol) and 1,2,3,4-tetrahydroisoquinoline (0.038 ml, 0.300 mmol) was stirred at 75° C. for 6 hrs under N₂. The reaction mixture was cooled to r.t., and EtOH (Volume: 3 ml) and SODIUM CYANOBOROHYDRIDE (0.057 g, 0.901 mmol) were added in sequence. The reaction mixture was stirred at r.t. for 3 days, and quenched with water. The resulting white inorganic solid was separated by filtration and washed with DCM. The organic layer was dried over MgSO₄ and concentrated. The crude product was purified by reverse phase chromatography to afford COMPOUND 226.

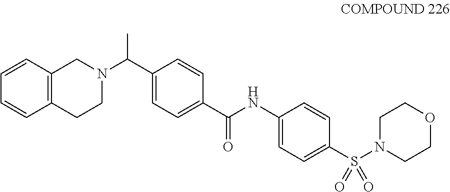 [SMILES: CC(c1ccc(C(=O)Nc2ccc(S(=O)(=O)N3CCOCC3)cc2)cc1)N1CCc2ccccc2C1]

¹H NMR (400 MHz, DMSO-d6) δ 10.75 (s, 1H), 8.09 (t, J=8.2 Hz, 4H), 7.76 (dd, J=8.5, 6.1 Hz, 4H), 7.27 (dd, J=18.4, 6.8 Hz, 4H), 4.85-4.67 (m, 2H), 4.30-4.07 (m, 1H), 3.64 (t, J=4.7 Hz, 4H), 2.87 (t, J=4.6 Hz, 4H), 1.76 (dd, J=11.2, 7.0 Hz, 4H), 1.28 (dd, J=43.3, 6.7 Hz, 3H); Method 1, retention time: 4.073 min; HRMS: m/z (M+H)⁺=506.2109 (Calculated for C₂₈H₃₂N₃O₄S=506.2108).

COMPOUND 119 was prepared according to the method described in Scheme 28 substituting 3-(morpholinosulfonyl)aniline for 4-(piperidin-1-ylsulfonyl)aniline.

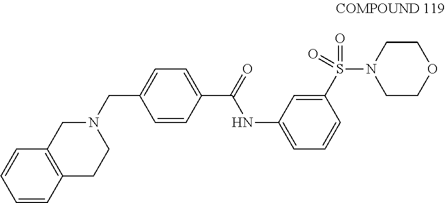 [SMILES: O=C(Nc1cccc(S(=O)(=O)N2CCOCC2)c1)c1ccc(CN2CCc3ccccc3C2)cc1]

¹H NMR (400 MHz, DMSO-d₆) δ 10.67 (s, 1H), 10.25 (s, 1H), 8.25 (t, J=1.9 Hz, 1H), 8.17-8.06 (m, 3H), 7.75-7.61 (m, 3H), 7.50-7.42 (m, 1H), 7.26 (d, J=11.2 Hz, 3H), 7.20 (s, 1H), 4.56 (s, 2H), 4.37 (s, 2H), 3.63 (dd, J=5.6, 3.8 Hz, 4H), 3.10 (s, 2H), 2.93-2.85 (m, 4H); HRMS: m/z (M+H)⁺=492.1943 (Calculated for C₂₇H₃₀N₃O₄S=492.1952).

COMPOUND 149 was prepared according to the method described in Scheme 28 substituting 4-morpholinoaniline for 4-(piperidin-1-ylsulfonyl)aniline.

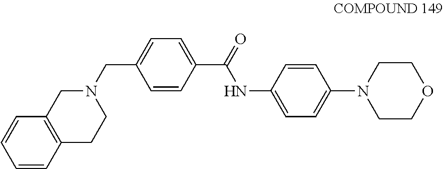 [SMILES: O=C(Nc1ccc(N2CCOCC2)cc1)c1ccc(CN2CCc3ccccc3C2)cc1]

¹H NMR (400 MHz, DMSO-d₆) δ 10.21 (s, 1H), 10.12 (s, 1H), 8.04 (d, J=8.0 Hz, 2H), 7.72-7.58 (m, 4H), 7.32-7.16 (m, 4H), 6.98-6.89 (m, 2H), 4.54 (s, 2H), 4.36 (s, 2H), 3.76-3.69 (m, 4H), 3.36 (m, 2H), 3.12-3.02 (m, 6H); HRMS: m/z (M+H)⁺=428.2342 (Calculated for C₂₇H₃₀N₃O₂=428.2333).

COMPOUND 143 was prepared according to the method described in Scheme 28 substituting aniline for 4-(piperidin-1-ylsulfonyl)aniline.

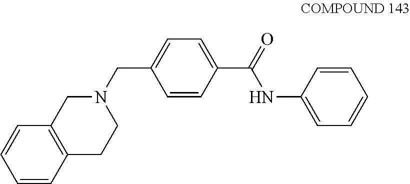 [SMILES: O=C(Nc1ccccc1)c1ccc(CN2CCc3ccccc3C2)cc1]

¹H NMR (400 MHz, DMSO-d₆) δ 10.29 (s, 1H), 10.22 (s, 1H), 8.05 (s, 2H), 7.80-7.73 (m, 2H), 7.69 (s, 2H), 7.35 (t, J=7.7 Hz, 2H), 7.24 (s, 4H), 7.10 (t, J=7.4 Hz, 1H), 4.53 (s, 2H), 4.34 (s, 2H), 3.08 (s, 2H); HRMS: m/z (M+H)⁺=343.1813 (Calculated for C₂₃H₂₃N₂O=343.1805).

COMPOUND 51 was prepared according to the method described in Scheme 8 substituting both the acid chloride and the amine.

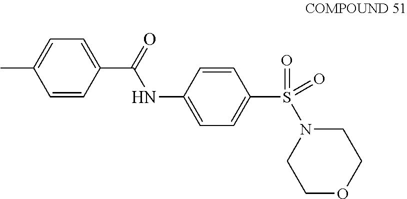 [SMILES: Cc1ccc(C(=O)Nc2ccc(S(=O)(=O)N3CCOCC3)cc2)cc1]

¹H NMR (400 MHz, DMSO-d₆) δ 10.55 (s, 1H), 8.10-8.01 (m, 2H), 7.92-7.84 (m, 2H), 7.75-7.67 (m, 2H), 7.39-7.31 (m, 2H), 3.61 (dd, J=5.8, 3.6 Hz, 4H), 2.88-2.81 (m, 4H), 2.38 (s, 3H); HRMS: m/z (M+H)⁺=361.1212 (Calculated for C₁₈H₂₁N₂O₄S=361.1217).

COMPOUND 163 was prepared according to the method described in Scheme 28 substituting 4-(morpholinomethyl)aniline for 4-(piperidin-1-ylsulfonyl)aniline.

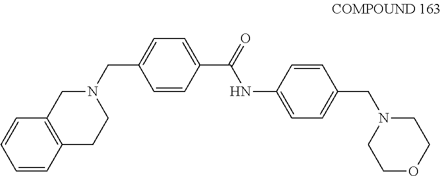 [SMILES: O=C(Nc1ccc(CN2CCOCC2)cc1)c1ccc(CN2CCc3ccccc3C2)cc1]

¹H NMR (400 MHz, DMSO-d₆) δ 10.18 (s, 1H), 7.91 (d, J=7.8 Hz, 2H), 7.71 (d, J=7.8 Hz, 2H), 7.50 (d, J=7.8 Hz, 2H), 7.26 (s, 2H), 7.09 (s, 3H), 7.00 (s, 1H), 3.72 (s, 2H), 3.55 (s, 6H), 3.41 (s, 2H), 2.81 (s, 2H), 2.68 (s, 2H), 2.33 (s, 4H); HRMS: m/z (M+H)⁺=442.2494 (Calculated for C₂₈H₃₁N₃O₂=442.2489).

COMPOUND 147 was prepared according to the method described in Scheme 28 substituting (4-aminophenyl)(morpholino)methanone for 4-(piperidin-1-ylsulfonyl)aniline.

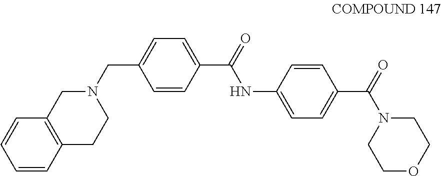 [SMILES: O=C(Nc1ccc(C(=O)N2CCOCC2)cc1)c1ccc(CN2CCc3ccccc3C2)cc1]

¹H NMR (400 MHz, DMSO-d₆) δ 10.48 (s, 1H), 10.23 (s, 1H), 8.07 (d, J=7.7 Hz, 2H), 7.89-7.81 (m, 2H), 7.71 (d, J=7.7 Hz, 2H), 7.47-7.38 (m, 2H), 7.32-7.16 (m, 4H), 4.56 (s, 2H), 4.37 (s, 2H), 3.80-3.25 (m, 10H), 3.10 (s, 2H); HRMS: m/z (M+H)⁺=456.2272 (Calculated for C₂₈H₃₀N₃O₃=456.2282).

COMPOUND 144 was prepared according to the method described in Scheme 28 substituting tert-butyl 4-((4-aminophenyl)sulfonyl)piperazine-1-carboxylate for 4-(piperidin-1-ylsulfonyl)aniline.

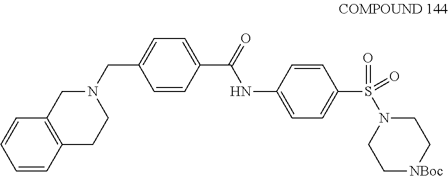 [SMILES: CC(C)(C)OC(=O)N1CCN(S(=O)(=O)c2ccc(NC(=O)c3ccc(CN4CCc5ccccc5C4)cc3)cc2)CC1]

¹H NMR (400 MHz, DMSO-d₆) δ 10.72 (s, 1H), 10.24 (s, 1H), 8.07 (dd, J=8.0, 5.8 Hz, 4H), 7.77-7.69 (m, 3H), 7.33-7.15 (m, 5H), 4.56 (s, 2H), 4.37 (s, 2H), 3.67 (s, 2H), 3.10 (s, 2H), 2.88-2.78 (m, 4H), 2.76-2.68 (m, 1H), 1.32 (s, 9H); HRMS: m/z (M+H)⁺=591.2631 (Calculated for C₃₂H₃₉N₄O₅S=591.2636).

COMPOUND 153 was prepared according to the method described in Scheme 28 substituting 4-amino-N,N-diethylbenzenesulfonamide for 4-(piperidin-1-ylsulfonyl)aniline.

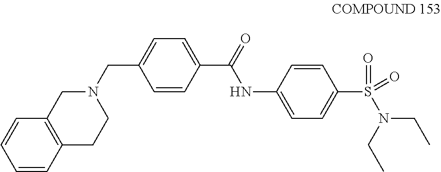 [SMILES: CCN(CC)S(=O)(=O)c1ccc(NC(=O)c2ccc(CN3CCc4ccccc4C3)cc2)cc1]

¹H NMR (400 MHz, DMSO-d₆) δ 10.66 (s, 1H), 10.22 (s, 1H), 8.07 (d, J=7.8 Hz, 2H), 8.04-7.95 (m, 2H), 7.82-7.68 (m, 4H), 7.33-7.15 (m, 4H), 4.56 (s, 2H), 4.37 (s, 2H), 3.67 (m, 2H), 3.14 (q, J=7.1 Hz, 4H), 3.14-3.05 (m, 2H), 1.03 (t, J=7.1 Hz, 6H); HRMS: m/z (M+H)⁺=478.2147 (Calculated for C₂₇H₃₂N₃O₃S=478.2159).

COMPOUND 148 was prepared according to the method described in Scheme 28 substituting 4-amino-N,N-dimethylbenzenesulfonamide for 4-(piperidin-1-ylsulfonyl)aniline.

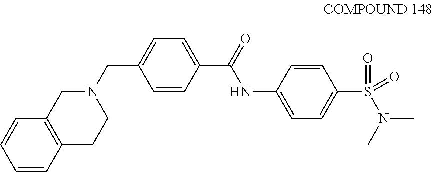 [SMILES: CN(C)S(=O)(=O)c1ccc(NC(=O)c2ccc(CN3CCc4ccccc4C3)cc2)cc1]

¹H NMR (400 MHz, DMSO-d₆) δ 10.70 (s, 1H), 10.24 (s, 1H), 8.11-8.01 (m, 4H), 7.79-7.69 (m, 4H), 7.30-7.18 (m, 4H), 4.56 (s, 2H), 4.37 (s, 2H), 3.67 (m, 2H), 3.10 (s, 2H), 2.59 (s, 6H); HRMS: m/z (M+H)⁺=450.1853 (Calculated for C₂₅H₂₈N₃O₃S=450.1846).

COMPOUND 154 was prepared according to the method described in Scheme 15 starting with the N-boc piperazine starting material, COMPOUND 144.

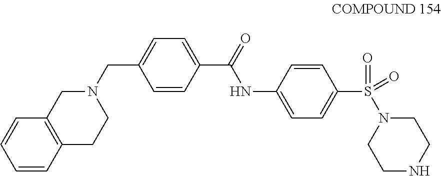 [SMILES: O=C(Nc1ccc(S(=O)(=O)N2CCNCC2)cc1)c1ccc(CN2CCc3ccccc3C2)cc1]

HRMS: m/z (M+H)⁺=491.2110 (Calculated for C₂₇H₃₁N₄O₃S=491.2111).

COMPOUND 146 was prepared according to the method described in Scheme 28 substituting 4-((4-methylpiperazin-1-yl)sulfonyl)aniline for 4-(piperidin-1-ylsulfonyl)aniline.

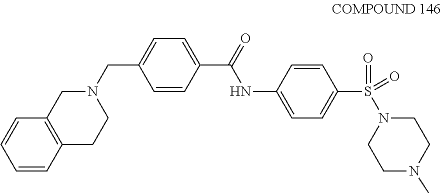 [SMILES: CN1CCN(S(=O)(=O)c2ccc(NC(=O)c3ccc(CN4CCc5ccccc5C4)cc3)cc2)CC1]

HRMS: m/z (M+H)⁺=507.2259 (Calculated for C₂₈H₃₃N₄O₃S=507.2283).

COMPOUND 155 was prepared according to the method described in Scheme 28 substituting 4-((2,6-dimethylmorpholino)sulfonyl)aniline for 4-(piperidin-1-ylsulfonyl)aniline.

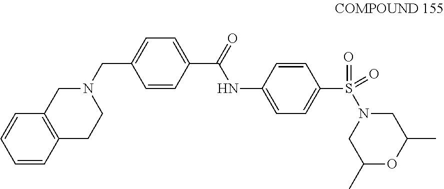 [SMILES: CC1CN(S(=O)(=O)c2ccc(NC(=O)c3ccc(CN4CCc5ccccc5C4)cc3)cc2)CC(C)O1]

¹H NMR (400 MHz, DMSO-d₆) δ 10.73 (s, 1H), 10.21 (s, 1H), 8.07 (dd, J=8.5, 6.5 Hz, 4H), 7.78-7.69 (m, 4H), 7.35-7.15 (m, 4H), 4.57 (s, 2H), 4.37 (s, 2H), 3.68 (m, 2H), 3.59 (ddd, J=10.3, 6.3, 2.3 Hz, 2H), 3.49 (d, J=10.7 Hz, 2H), 3.10 (s, 2H), 1.87-1.76 (m, 2H), 1.04 (d, J=6.2 Hz, 6H); HRMS: m/z (M+H)⁺=520.2283 (Calculated for C₂₉H₃₄N₃O₄S=520.2265).

COMPOUND 145 was prepared according to the method described in Scheme 28 substituting 4-(pyrrolidin-1-ylsulfonyl)aniline for 4-(piperidin-1-ylsulfonyl)aniline.

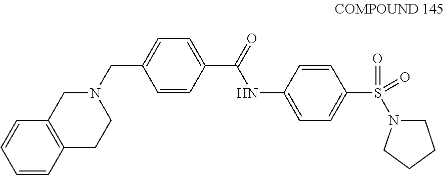 [SMILES: O=C(Nc1ccc(S(=O)(=O)N2CCCC2)cc1)c1ccc(CN2CCc3ccccc3C2)cc1]

¹H NMR (400 MHz, DMSO-d₆) δ 10.68 (s, 1H), 10.25 (s, 1H), 8.11-7.99 (m, 4H), 7.84-7.76 (m, 2H), 7.72 (d, J=7.7 Hz, 2H), 7.33-7.15 (m, 4H), 4.56 (s, 2H), 4.37 (s, 2H), 3.67 (m, 2H), 3.17-3.09 (m, 6H), 1.68-1.58 (m, 4H); HRMS: m/z (M+H)⁺=476.2012 (Calculated for C₂₇H₃₀N₃O₃S=476.2002).

COMPOUND 178 was prepared according to the method described in Scheme 28 substituting 4-(trifluoromethoxy)aniline for 4-(piperidin-1-ylsulfonyl)aniline.

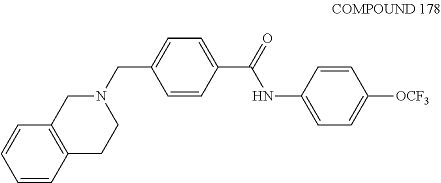 [SMILES: O=C(Nc1ccc(OC(F)(F)F)cc1)c1ccc(CN2CCc3ccccc3C2)cc1]

¹H NMR (400 MHz, DMSO-d₆) δ 10.49 (s, 1H), 10.25 (s, 1H), 8.06 (d, J=8.0 Hz, 2H), 7.93-7.84 (m, 2H), 7.69 (dd, J=16.2, 8.4 Hz, 2H), 7.37 (d, J=8.6 Hz, 2H), 7.29-7.18 (m, 4H), 4.56 (s, 2H), 4.36 (s, 2H), 3.67 (m, 2H), 3.10 (s, 2H); HRMS: m/z (M+H)⁺=427.1612 (Calculated for C₂₄H₂₂F₃N₂O₂=427.1628).

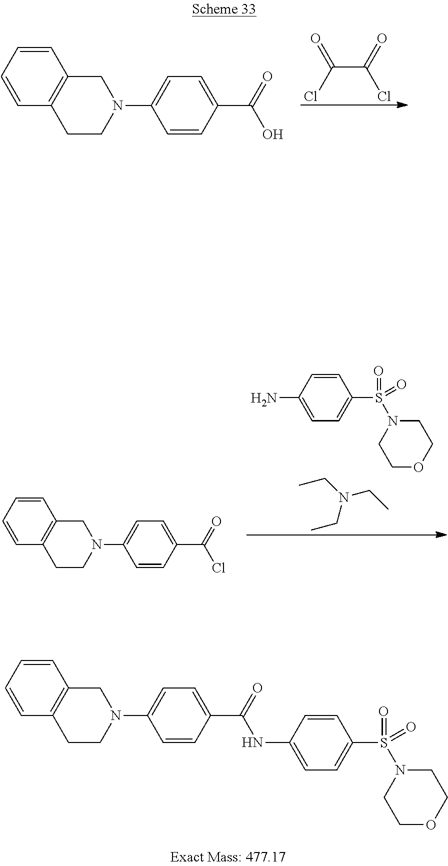

Scheme 33 To a solution of 4-(3,4-dihydroisoquinolin-2(1H)-yl)benzoic acid (35 mg, 0.14 mmol) in DCM (2 mL) was added oxalyl chloride (0.06 mL, 0.7 mmol) followed by DMF (2 drops). The acid became soluble and this solution stirred at rt 1.25 hr. The reaction mixture was concentrated under a stream of argon. To the reaction residue was added DCM (2 mL) followed by triethylamine (0.077 mL, 0.55 mmol) and finally 4-(morpholinosulfonyl)aniline (34 mg, 0.14 mmol). The reaction mixture stirred at rt 2 hr, was concentrated under a stream of air, and subsequently purified by reverse phase chromatography to afford COMPOUND 152.

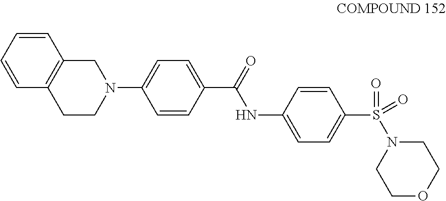 [SMILES: O=C(Nc1ccc(S(=O)(=O)N2CCOCC2)cc1)c1ccc(N2CCc3ccccc3C2)cc1]

¹H NMR (400 MHz, DMSO-d₆) δ 10.31 (s, 1H), 8.10-8.01 (m, 2H), 7.94-7.86 (m, 2H), 7.72-7.64 (m, 2H), 7.29-7.14 (m, 4H), 7.09-7.01 (m, 2H), 4.54 (s, 2H), 3.70-3.58 (m, 6H), 2.93 (t, J=5.9 Hz, 2H), 2.87-2.80 (m, 4H); HRMS: m/z (M+Na)⁺=500.1611 (Calculated for C₂₆H₂₇N₃NaO₄S=500.1614).

COMPOUND 168 was prepared according to the methods described in Schemes 26 and 27. The acid component, 4-((benzyl(methyl)amino)methyl)benzoic acid, was prepared similarly to that of the acid resulting from step 1 of Scheme 27, and used subsequently in a step similar to that of Scheme 28.

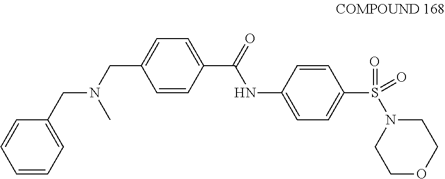 [SMILES: CN(Cc1ccccc1)Cc1ccc(C(=O)Nc2ccc(S(=O)(=O)N3CCOCC3)cc2)cc1]

¹H NMR (400 MHz, DMSO-d₆) δ 10.73 (s, 1H), 9.93 (s, 1H), 8.10-8.02 (m, 4H), 7.71 (dd, J=18.2, 8.4 Hz, 4H), 7.50 (d, J=10.3 Hz, 5H), 4.50 (dd, J=27.4, 12.5 Hz, 2H), 4.35-4.23 (m, 2H), 3.62 (dd, J=5.6, 3.8 Hz, 4H), 2.88-2.81 (m, 4H), 2.60-2.50 (m, 3H); HRMS: m/z (M+H)⁺=480.1974 (Calculated for C₂₆H₃₀N₃O₄S=480.1952).

COMPOUND 172 was prepared according to the methods described in Schemes 26 and 27. The acid component, 4-((3,4-dihydroquinolin-1(2H)-yl)methyl)benzoic acid, was prepared similarly to that of the acid resulting from step 1 of Scheme 27, and used subsequently in a step similar to that of Scheme 28.

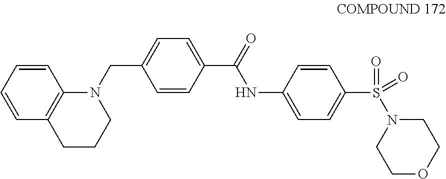 [SMILES: O=C(Nc1ccc(S(=O)(=O)N2CCOCC2)cc1)c1ccc(CN2CCCc3ccccc32)cc1]

¹H NMR (400 MHz, DMSO-d6) δ 10.59 (s, 1H), 8.10-7.99 (m, 2H), 7.95-7.85 (m, 2H), 7.78-7.66 (m, 2H), 7.40 (dq, J=7.8, 0.8 Hz, 2H), 6.93-6.80 (m, 2H), 6.50-6.36 (m, 2H), 4.55 (s, 2H), 3.65-3.57 (m, 4H), 3.47-3.29 (m, 2H), 2.87-2.80 (m, 4H), 2.74 (t, J=6.3 Hz, 2H), 1.99-1.88 (m, 2H); HRMS: m/z (M+H)⁺=492.1965 (Calculated for C₂₇H₃₀N₃O₄S=492.1952).

COMPOUND 174 was prepared according to the method described in Scheme 29 substituting 2-methoxy-N-methylethanamine for 1-(piperazin-1-yl)ethanone.

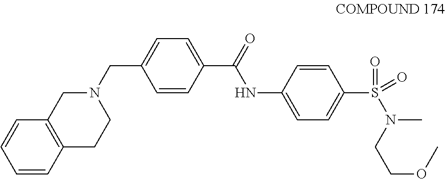 [SMILES: COCCN(C)S(=O)(=O)c1ccc(NC(=O)c2ccc(CN3CCc4ccccc4C3)cc2)cc1]

¹H NMR (400 MHz, DMSO-d₆) δ 10.68 (s, 1H), 10.22 (s, 1H), 8.11-7.98 (m, 4H), 7.75 (dd, J=18.5, 8.2 Hz, 4H), 7.25 (m, 4H), 4.56 (s, 2H), 4.37 (s, 2H), 3.67 (m, 2H), 3.44 (t, J=5.6 Hz, 2H), 3.22 (s, 3H), 3.11 (t, J=5.6 Hz, 4H), 2.69 (s, 3H); HRMS: m/z (M+H)⁺=494.2103 (Calculated for C₂₇H₃₂N₃O₄S=494.2108).

COMPOUND 166 was prepared according to the methods described in Schemes 26 and 27. The acid component, 4-(2-(3,4-dihydroisoquinolin-2(1H)-yl)ethyl)benzoic acid, was prepared similarly to that of the acid resulting from step 1 of Scheme 27, and used subsequently in a step similar to that of Scheme 28.

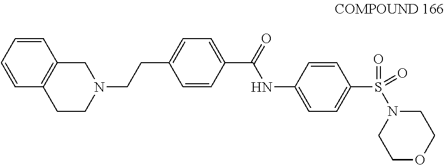 [SMILES: O=C(Nc1ccc(S(=O)(=O)N2CCOCC2)cc1)c1ccc(CCN2CCc3ccccc3C2)cc1]

¹H NMR (400 MHz, DMSO-d₆) δ 10.62 (s, 1H), 9.97 (s, 1H), 8.11-8.02 (m, 2H), 7.98 (d, J=8.1 Hz, 2H), 7.77-7.68 (m, 2H), 7.53-7.46 (m, 2H), 7.28 (d, J=6.7 Hz, 3H), 7.21 (d, J=6.9 Hz, 1H), 4.65 (d, J=15.4 Hz, 1H), 4.43 (d, J=10.6 Hz, 1H), 3.81 (s, 1H), 3.62 (dd, J=5.6, 3.8 Hz, 4H), 3.52 (d, J=10.3 Hz, 2H), 3.20 (d, J=8.2 Hz, 3H), 3.11 (s, 2H), 2.88-2.81 (m, 4H); HRMS: m/z (M+H)⁺=506.2094 (Calculated for C₂₈H₃₂N₃O₄S=506.2108).

COMPOUND 179 was prepared according to the method described in Scheme 28 substituting 4-(piperidin-1-ylmethyl)benzoic acid for the acid.

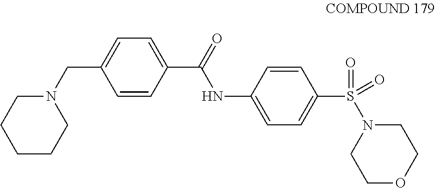 [SMILES: O=C(Nc1ccc(S(=O)(=O)N2CCOCC2)cc1)c1ccc(CN2CCCCC2)cc1]

HRMS: m/z (M+H)⁺=444.1942 (Calculated for C₂₃H₃₀N₃O₄S=444.1952).

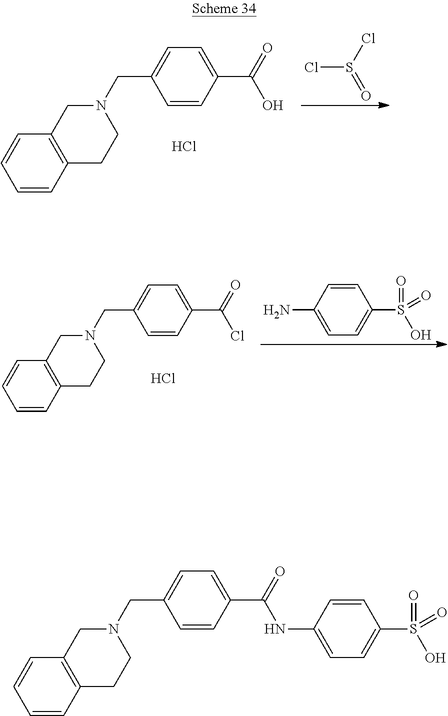

### Scheme 34

To 4-((3,4-dihydroisoquinolin-2(1H)-yl)methyl)benzoic acid hydrochloride (1.13 g, 3.7 mmol) was slowly added thionyl chloride (4.3 mL, 59 mmol). When most of the gas was evolved the solution was heated at 60° C. overnight. In the morning, the reaction had become a slurry. The excess thionyl chloride was removed by blowing down with a stream of argon. The slurry was rediluted 2× with EtOAc and reconcentrated to yield a light yellow solid. This solid was slurryed in pyridine (5 mL) and treated with 4-aminobenzenesulfonic acid (0.71 g, 4.1 mmol) followed by DMF (5 mL). The thick pink slurry was sonicated to generate a homogeneous slurry and stirred at rt 2 hr. An additional 3 mL of DMF was added and the reaction mixture was heated at 60° C. 1 hr. The mixture was diluted with water and filtered, washing with ethanol and ethylacetate. This was sufficiently pure to use in subsequent synthetic steps. A small amount was also purified by reverse phase chromatography to afford COMPOUND 156.

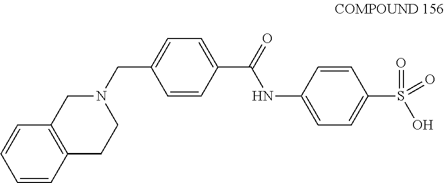 [SMILES: O=C(Nc1ccc(S(=O)(=O)O)cc1)c1ccc(CN2CCc3ccccc3C2)cc1]

¹H NMR (400 MHz, DMSO-d₆) δ 10.35 (s, 1H), 10.15 (s, 1H), 8.07 (d, J=7.9 Hz, 2H), 7.70 (dt, J=7.1, 2.1 Hz, 4H), 7.60-7.52 (m, 2H), 7.24 (dd, J=16.5, 10.4 Hz, 4H), 4.55 (s, 2H), 4.36 (s, 2H), 3.68 (m, 2H), 3.10 (s, 2H); HRMS: m/z (M+H)=423.1365 (Calculated for C₂₃H₂₃N₂O₄S=423.1373).

COMPOUND 188 was prepared according to the method described in Scheme 29 substituting 4,4-difluoropiperidine hydrochloride for 1-(piperazin-1-yl)ethanone.

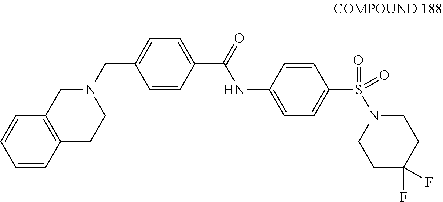 [SMILES: O=C(Nc1ccc(S(=O)(=O)N2CCC(F)(F)CC2)cc1)c1ccc(CN2CCc3ccccc3C2)cc1]

¹H NMR (400 MHz, DMSO-d₆) δ 10.73 (s, 1H), 10.23 (s, 1H), 8.11-8.02 (m, 4H), 7.83-7.69 (m, 4H), 7.35-7.15 (m, 4H), 4.56 (d, J=4.5 Hz, 2H), 4.37 (s, 2H), 3.67 (m, 2H), 3.15-3.00 (m, 6H), 2.04 (td, J=13.5, 6.5 Hz, 4H); HRMS: m/z (M+H)=526.1967 (Calculated for C₂₈H₃₀F₂N₃O₃S=526.1970).

COMPOUND 165 was prepared according to the method described in Scheme 29 substituting 3-phenylmorpholine for 1-(piperazin-1-yl)ethanone.

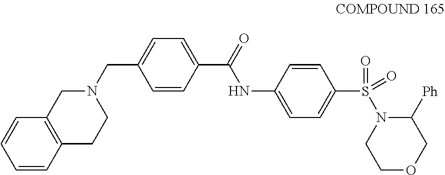 [SMILES: O=C(Nc1ccc(S(=O)(=O)N2CCOCC2c2ccccc2)cc1)c1ccc(CN2CCc3ccccc3C2)cc1]

¹H NMR (400 MHz, DMSO-d₆) δ 10.69 (s, 1H), 10.27 (s, 1H), 8.08 (d, J=7.8 Hz, 2H), 8.04-7.95 (m, 2H), 7.84-7.69 (m, 4H), 7.47-7.16 (m, 9H), 4.83 (s, 1H), 4.57 (s, 2H), 4.37 (s, 2H), 4.11 (dd, J=12.1, 2.0 Hz, 1H), 3.74-3.62 (m, 2H), 3.53-3.42 (m, 2H), 3.27-3.08 (m, 5H); HRMS: m/z (M+H)⁺=568.2274 (Calculated for C₃₃H₃₄N₃O₄S=568.2265).

COMPOUND 169 was prepared according to the method described in Scheme 29 substituting 2-methylmorpholine hydrochloride for 1-(piperazin-1-yl)ethanone.

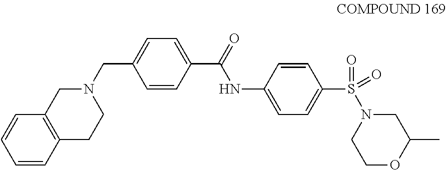 [SMILES: CC1CN(S(=O)(=O)c2ccc(NC(=O)c3ccc(CN4CCc5ccccc5C4)cc3)cc2)CCO1]

¹H NMR (400 MHz, DMSO-d₆) δ 10.73 (s, 1H), 10.26 (s, 1H), 8.11-8.02 (m, 4H), 7.78-7.69 (m, 4H), 7.33-7.15 (m, 4H), 4.57 (s, 2H), 4.37 (s, 2H), 3.85-3.77 (m, 1H), 3.67 (m, 2H), 3.59-3.37 (m, 4H), 3.10 (s, 2H), 2.52 (s, 1H), 2.22 (td, J=11.4, 3.3 Hz, 1H), 1.89 (dd, J=11.4, 10.0 Hz, 1H), 1.04 (d, J=6.2 Hz, 3H); HRMS: m/z (M+H)⁺=506.2112 (Calculated for C₂₈H₃₂N₃O₄S=506.2108).

COMPOUND 171 was prepared according to the method described in Scheme 29 substituting 2-phenylmorpholine for 1-(piperazin-1-yl)ethanone.

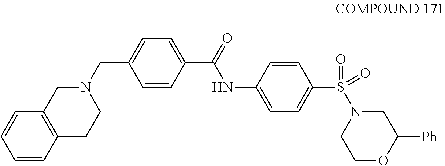 [SMILES: O=C(Nc1ccc(S(=O)(=O)N2CCOC(c3ccccc3)C2)cc1)c1ccc(CN2CCc3ccccc3C2)cc1]

¹H NMR (400 MHz, DMSO-d₆) δ 10.72 (s, 1H), 10.26 (s, 1H), 8.10-8.00 (m, 4H), 7.41-7.37 (m, 1H), 7.34-7.17 (m, 8H), 4.58 (dd, J=10.3, 2.6 Hz, 2H), 4.37 (s, 2H), 4.06-3.98 (m, 1H), 3.76-3.29 (m, 6H), 3.10 (s, 2H), 2.47-2.32 (m, 1H), 2.12 (dd, J=11.5, 10.3 Hz, 1H); HRMS: m/z (M+H)⁺=568.2263 (Calculated for C₃₃H₃₄N₃O₄S=568.2265).

COMPOUND 176 was prepared according to the method described in Scheme 29 substituting tetrahydro-2H-pyran-4-amine hydrochloride for 1-(piperazin-1-yl)ethanone.

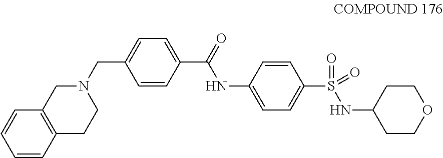 [SMILES: O=C(Nc1ccc(S(=O)(=O)NC2CCOCC2)cc1)c1ccc(CN2CCc3ccccc3C2)cc1]

¹H NMR (400 MHz, DMSO-d₆) δ 10.64 (s, 1H), 10.23 (s, 1H), 8.07 (d, J=7.7 Hz, 2H), 8.01-7.93 (m, 2H), 7.80 (d, J=8.7 Hz, 2H), 7.71 (dd, J=12.4, 7.5 Hz, 3H), 7.33-7.15 (m, 4H), 4.56 (s, 2H), 4.37 (s, 2H), 3.70 (dt, J=11.8, 3.8 Hz, 3H), 3.26-3.06 (m, 5H), 1.50 (d, J=12.6 Hz, 2H), 1.41-1.26 (m, 2H); HRMS: m/z (M+H)⁺=506.2114 (Calculated for C₂₈H₃₂N₃O₄S=506.2108).

COMPOUND 167 was prepared according to the method described in Scheme 29 substituting N-methyltetrahydro-2H-pyran-4-amine for 1-(piperazin-1-yl)ethanone.

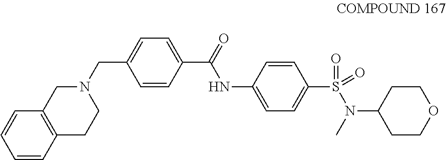 [SMILES: CN(C1CCOCC1)S(=O)(=O)c1ccc(NC(=O)c2ccc(CN3CCc4ccccc4C3)cc2)cc1]

¹H NMR (400 MHz, DMSO-d6) δ 10.67 (s, 1H), 10.22 (s, 1H), 8.10-7.96 (m, 4H), 7.86-7.77 (m, 2H), 7.72 (d, J=7.7 Hz, 2H), 7.33-7.15 (m 4H), 4.56 (s, 2H), 4.37 (d, J=6.5 Hz, 2H), 3.92 (ddt, J=11.9, 7.9, 4.1 Hz, 1H), 3.79 (dd, J=11.4, 4.4 Hz, 2H), 3.67 (s, 1H), 3.31 (td, J=11.8, 1.8 Hz, 2H), 3.10 (s, 2H), 2.68 (s, 3H), 1.68-1.52 (m, 2H), 1.24-1.16 (m, 2H); HRMS: m/z (M+H)⁺=520.2271 (Calculated for C₂₉H₃₄N₃O₄S=520.2265).

COMPOUND 173 was prepared according to the method described in Scheme 29 substituting N-methyloxetan-3-amine for 1-(piperazin-1-yl)ethanone.

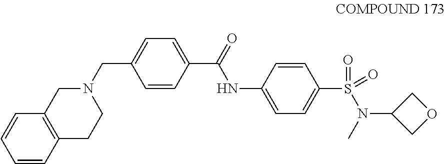 [SMILES: CN(C1COC1)S(=O)(=O)c1ccc(NC(=O)c2ccc(CN3CCc4ccccc4C3)cc2)cc1]

¹H NMR (400 MHz, DMSO-d6) δ 10.72 (s, 1H), 10.25 (s, 1H), 8.10-8.00 (m, 4H), 7.74 (dd, J=9.9, 7.9 Hz, 4H), 7.35-7.15 (m, 4H), 4.68-4.48 (m, 5H), 4.37 (s, 2H), 3.67 (s, 1H), 3.10 (s, 2H), 2.69 (s, 3H); HRMS: m/z (M+Na)⁺=514.1782 (Calculated for C₂₇H₂₉N₃NaO₄S=514.1771).

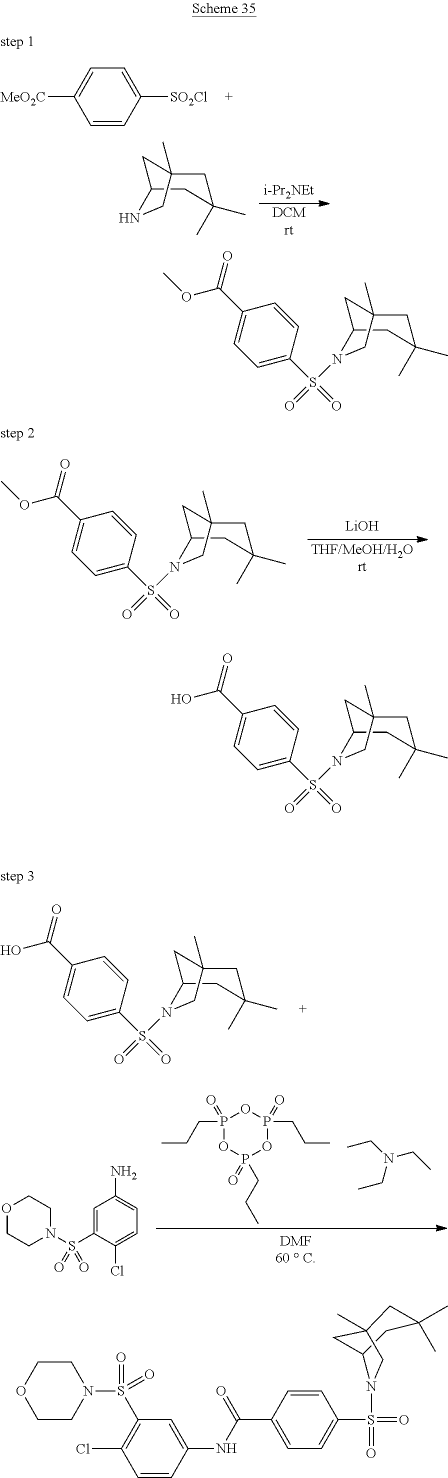

### Scheme 35, Step 1

To a solution of (1S,5R)-1,3,3-trimethyl-6-azabicyclo[3.2.1]octane (70 mg, 0.457 mmol) and N,N-diisopropylethylamine (160 μL, 0.913 mmol) in DCM (3 mL) was added methyl 4-(chlorosulfonyl)benzoate (107 mg, 0.457 mmol). The reaction mixture stirred at rt 17.5 hr. The reaction mixture was diluted with water and DCM. The layers were separated and the aqueous layer was reextracted with DCM. The combined organic layers were dried with MgSO₄ and concentrated in vacuo to afford methyl 4-(1,3,3-trimethyl-6-azabicyclo[3.2.1]octan-6-ylsulfonyl)benzoate as an oil; LCMS: m/z (M+H)⁺=352.2. Assumed quantitative conversion and used without purification in step 2 of Scheme 15.

### Scheme 35, Step 2

To a solution of methyl 4-(1,3,3-trimethyl-6-azabicyclo[3.2.1]octan-6-ylsulfonyl)benzoate (0.457 mmol) in a 3:1:1 solution of THF/methanol/water (5 ml) was added lithium hydroxide (55 mg, 2.3 mmol). The reaction mixture stirred at rt 1 hr. The reaction mixture was concentrated in vacuo, diluted with water, acidified with 1N HCl, and extracted with DCM (3×). The combined organic layers were dried with MgSO₄ and concentrated in vacuo to afford 4-(1,3,3-trimethyl-6-azabicyclo[3.2.1]octan-6-ylsulfonyl)benzoic acid as a colorless solid; LCMS: m/z (M+H)⁺=338.1. Assumed quantitative conversion and used without purification in step 3 of Scheme 15.

### Scheme 35, Step 3

To a solution of 4-(1,3,3-trimethyl-6-azabicyclo[3.2.1]octan-6-ylsulfonyl)benzoic acid (0.457 mmol), 4-chloro-3-(morpholinosulfonyl)aniline (0.126 g, 0.457 mmol), and triethylamine (0.318 mL, 2.285 mmol) in DMF (1.5 ml) was added 50 wt. % propylphosphonic anhydride solution in DMF (583 μL, 1.143 mmol). The mixture was stirred at rt for 10 min and then heated at 60° C. for 3 hr. The reaction mixture was cooled to rt and diluted with water and EtOAc. The layers were separated and the aqueous layer was reextracted with EtOAc. The combined organic layers were washed with water, dried with MgSO₄, and concentrated in vacuo to afford a residue. The residue was taken up in DMSO and subsequently purified by reverse phase chromatography to give Compound 3.

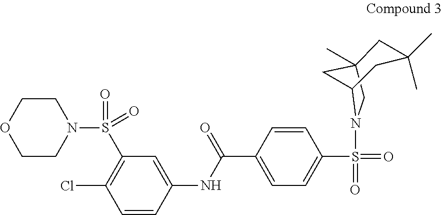 [SMILES: CC1(C)CC2CC(C)(CN2S(=O)(=O)c2ccc(C(=O)Nc3ccc(Cl)c(S(=O)(=O)N4CCOCC4)c3)cc2)C1]

¹H NMR (400 MHz, DMSO-d6) δ ppm 10.87 (s, 1H), 8.46 (d, J=2.6 Hz, 1H), 8.19-8.08 (m, 3H), 8.01-7.93 (m, 2H), 7.71 (d, J=8.7 Hz, 1H), 4.10 (ddd, J=5.9, 4.2, 1.7 Hz, 1H), 3.65-3.58 (m, 4H), 3.30-3.12 (m, 5H), 2.72 (dd, J=9.7, 1.6 Hz, 1H), 1.73-1.63 (m, 1H), 1.52-1.43 (m, 1H), 1.32 (dt, J=13.9, 1.7 Hz, 2H), 1.16 (s, 4H), 0.92-0.76 (m, 7H); HRMS: m/z (M+H)⁺=596.1643 (Calculated for C₇₁H₃₅ClN₃O₆S₂=596.1650).

COMPOUND 55 was prepared according to the method described in Scheme 35 step 3 substituting 4-(morpholinosulfonyl)benzoic acid for 4-(1,3,3-trimethyl-6-azabicyclo[3.2.1]octan-6-ylsulfonyl)benzoic acid.

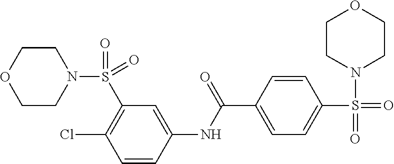 [SMILES: O=C(Nc1ccc(Cl)c(S(=O)(=O)N2CCOCC2)c1)c1ccc(S(=O)(=O)N2CCOCC2)cc1]

¹H NMR (400 MHz, DMSO-d₆) δ 10.90 (s, 1H), 8.46 (d, J=2.6 Hz, 1H), 8.23-8.08 (m, 3H), 7.94-7.86 (m, 2H), 7.72 (d, J=8.8 Hz, 1H), 3.62 (dt, J=7.1, 2.8 Hz, 8H), 3.21-3.14 (m, 4H), 2.94-2.87 (m, 4H); HRMS: m/z (M+H)⁺=530.0820 (Calculated for C₂₁H₂₅ClN₃O₇S₂=530.0817).

COMPOUND 52 was prepared according to the method described in Scheme 35 step 3 substituting 3-(morpholinosulfonyl)aniline for 4-chloro-3-(morpholinosulfonyl)aniline.

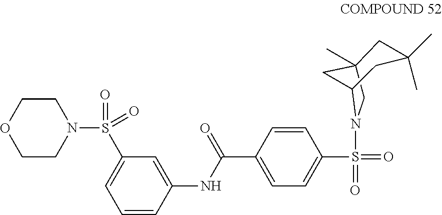 [SMILES: CC1(C)CC2CC(C)(CN2S(=O)(=O)c2ccc(C(=O)Nc3cccc(S(=O)(=O)N4CCOCC4)c3)cc2)C1]

¹H NMR (400 MHz, DMSO-d₆) δ 10.81 (s, 1H), 8.23 (t, J=2.0 Hz, 1H), 8.20-8.08 (m, 3H), 8.01-7.93 (m, 2H), 7.66 (t, J=8.0 Hz, 1H), 7.47 (ddd, J=7.8, 1.8, 1.0 Hz, 1H), 4.10 (ddd, J=5.9, 4.1, 1.7 Hz, 1H), 3.66-3.59 (m, 4H), 3.30-3.21 (m, 1H), 2.88 (dd, J=5.8, 3.7 Hz, 4H), 2.72 (dd, J=9.7, 1.7 Hz, 1H), 1.68 (dd, J=13.8, 4.1 Hz, 1H), 1.52-1.43 (m, 1H), 1.32 (dd, J=13.8, 1.9 Hz, 2H), 1.16 (m, 4H), 0.92-0.76 (m, 7H); HRMS: m/z (M+H)⁺=562.2035 (Calculated for C₂₇H₃₆N₃O₆S₂=562.2040).

COMPOUND 53 was prepared according to the method described in Scheme 35 step 3 substituting 4-chloroaniline for 4-chloro-3-(morpholinosulfonyl)aniline.

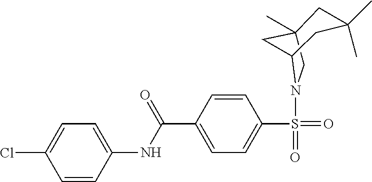 [SMILES: CC1(C)CC2CC(C)(CN2S(=O)(=O)c2ccc(C(=O)Nc3ccc(Cl)cc3)cc2)C1]

¹H NMR (400 MHz, DMSO-d6) δ 10.58 (s, 1H), 8.16-8.08 (m, 2H), 7.99-7.91 (m, 2H), 7.84-7.75 (m, 2H), 7.46-7.37 (m, 2H), 4.09 (ddd, J=5.9, 4.2, 1.7 Hz, 1H), 3.30-3.20 (m, 1H), 2.72 (dd, J=9.6, 1.7 Hz, 1H), 1.68 (dd, J=13.8, 4.2 Hz, 1H), 1.51-1.43 (m, 1H), 1.32 (dt, J=13.8, 1.8 Hz, 2H), 1.16 (m, 4H), 0.92-0.76 (m, 7H); HRMS: m/z (M+H)⁺=447.1498 (Calculated for C₂₃H₂₈ClN₂O₃S=447.1504).

COMPOUND 54 was prepared according to the method described in Scheme 35 step 3 substituting 4-(piperidin-lylsulfonyl)benzoic acid for 4-(1,3,3-trimethyl-6-azabicyclo[3.2.1]octan-6-ylsulfonyl)benzoic acid.

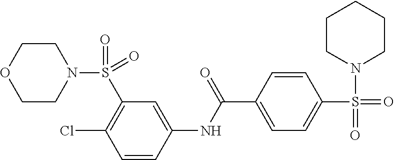 [SMILES: O=C(Nc1ccc(Cl)c(S(=O)(=O)N2CCOCC2)c1)c1ccc(S(=O)(=O)N2CCCCC2)cc1]

¹H NMR (400 MHz, DMSO-d6) δ 10.88 (s, 1H), 8.46 (d, J=2.5 Hz, 1H), 8.20-8.08 (m, 3H), 7.92-7.84 (m, 2H), 7.71 (d, J=8.7 Hz, 1H), 3.65-3.58 (m, 4H), 3.21-3.13 (m, 4H), 2.92 (t, J=5.4 Hz, 4H), 1.53 (dq, J=11.3, 5.8, 5.3 Hz, 4H), 1.36 (q, J=6.4, 6.0 Hz, 2H); HRMS: m/z (M+H)⁺=528.1019 (Calculated for C₂₂H₂₇ClN₃O₆S₂=528.1024).

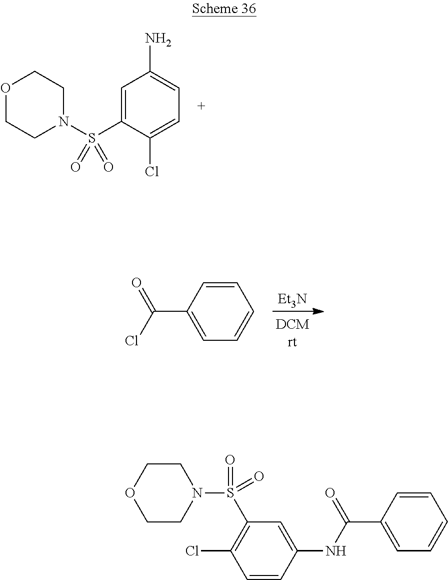

COMPOUND 62 To a solution of 4-chloro-3-(morpholinosulfonyl)aniline (60 mg, 0.217 mmol) and triethylamine (60 μL, 0.434 mmol) in DCM (3 ml) was added benzoyl chloride (30 μL, 0.238 mmol). The reaction mixture stirred at rt 1 hr. The reaction mixture was concentrated under a stream of air. The residue was taken up in DMSO and subsequently purified by reverse phase chromatography to give COMPOUND 62.

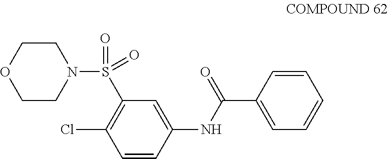 [SMILES: O=C(Nc1ccc(Cl)c(S(=O)(=O)N2CCOCC2)c1)c1ccccc1]

¹H NMR (400 MHz, DMSO-d₆) δ 10.66 (s, 1H), 8.48 (d, J=2.5 Hz, 1H), 8.12 (dt, J=8.7, 2.5 Hz, 1H), 8.00-7.92 (m, 2H), 7.72-7.49 (m, 4H), 3.65-3.58 (m, 4H), 3.21-3.13 (m, 4H); HRMS: m/z (M+H)⁺=381.0662 (Calculated for C₁₇HsClN₂O₄S=381.0670).

Additional compounds identified herein as CHEMOTYPE 1, CHEMOTYPE 2, CHEMOTYPE 3, CHEMOTYPE 4, AND CHEMOTYPE 5 also are provided herein:

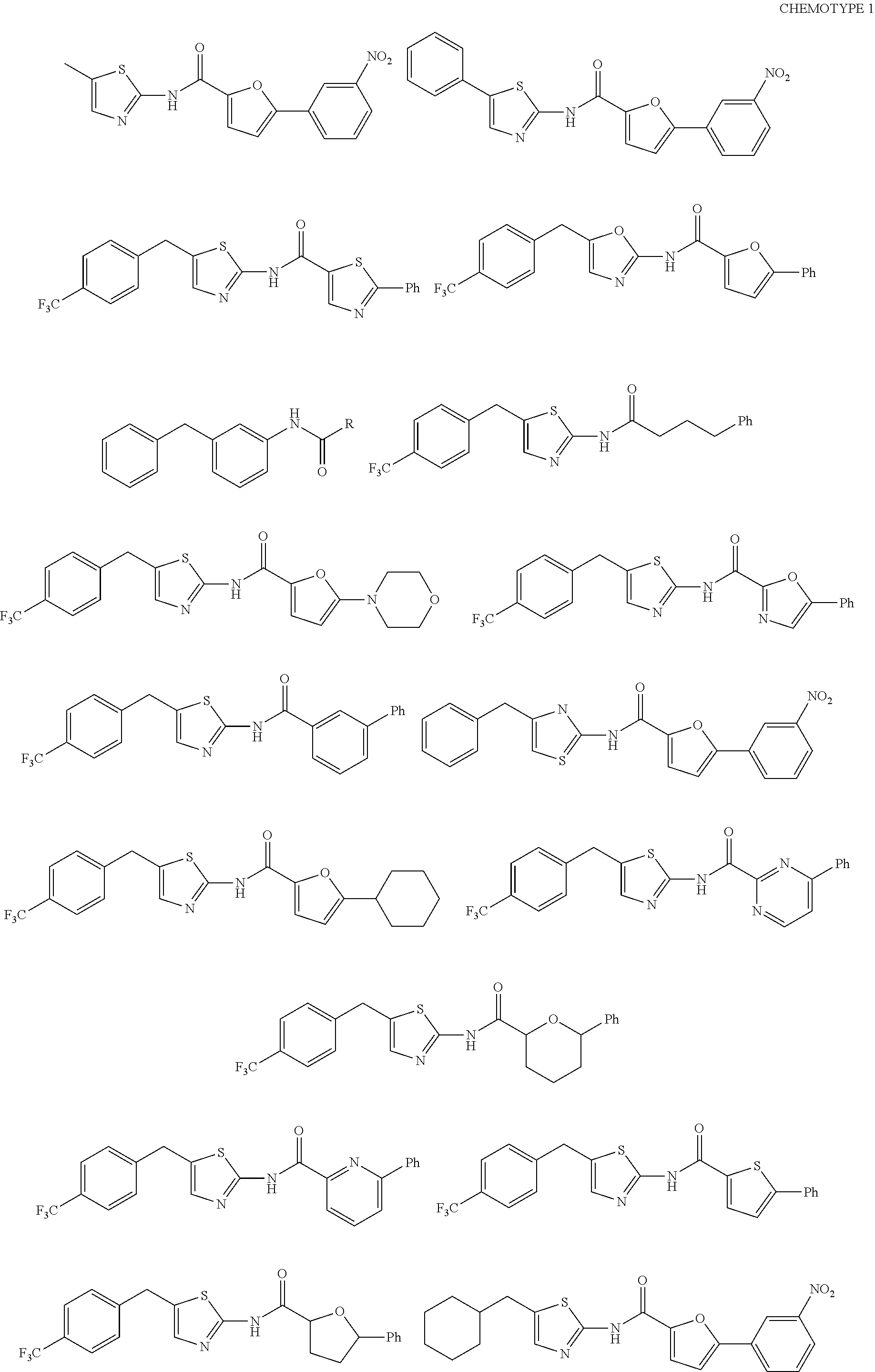

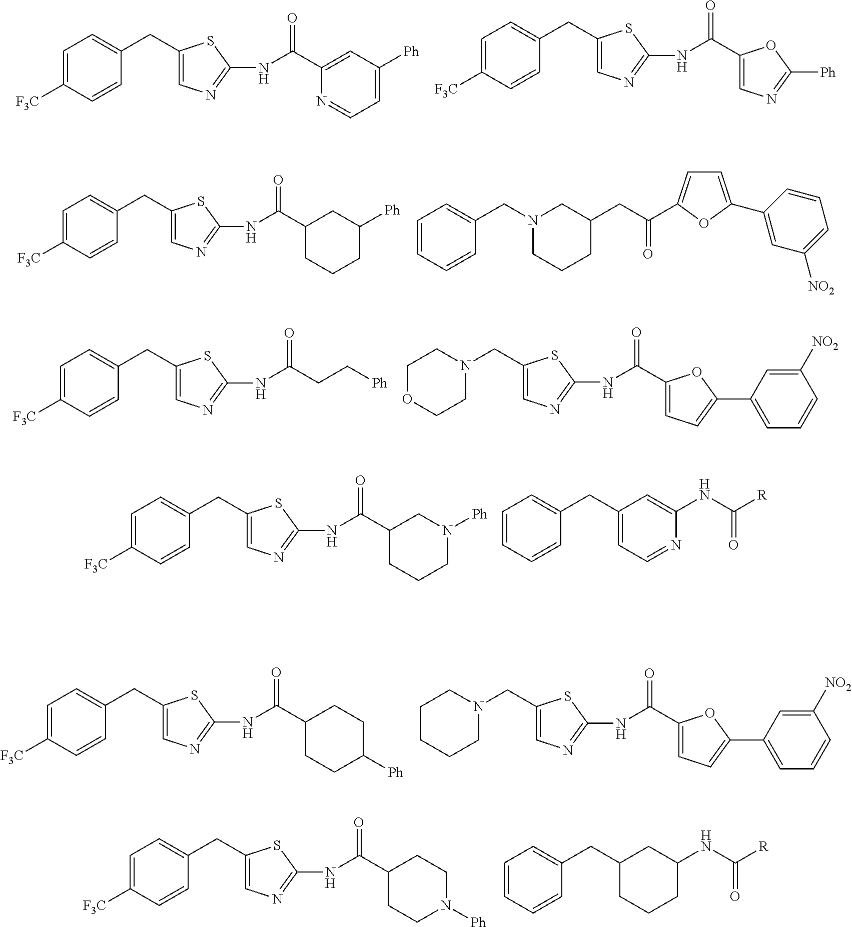

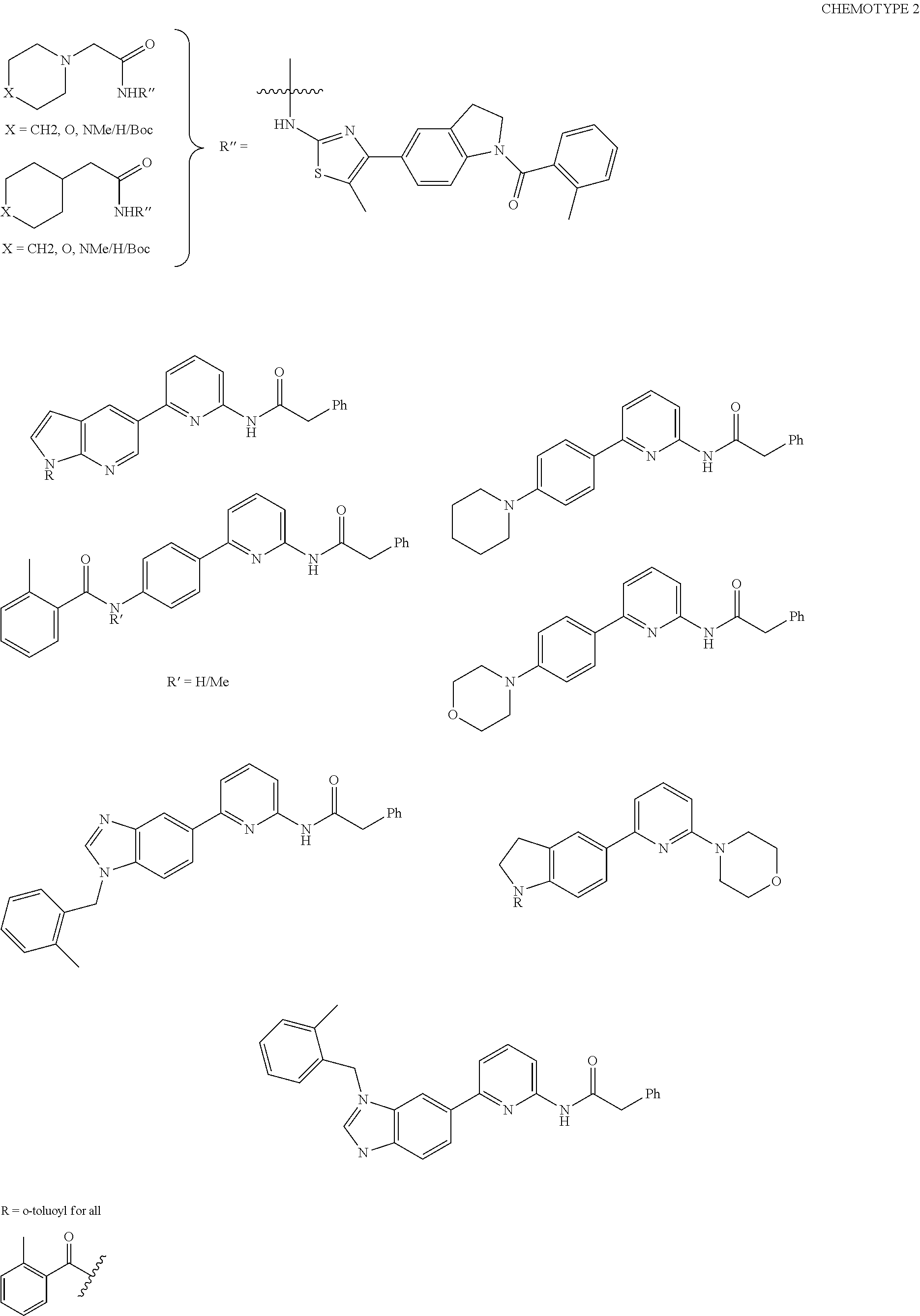

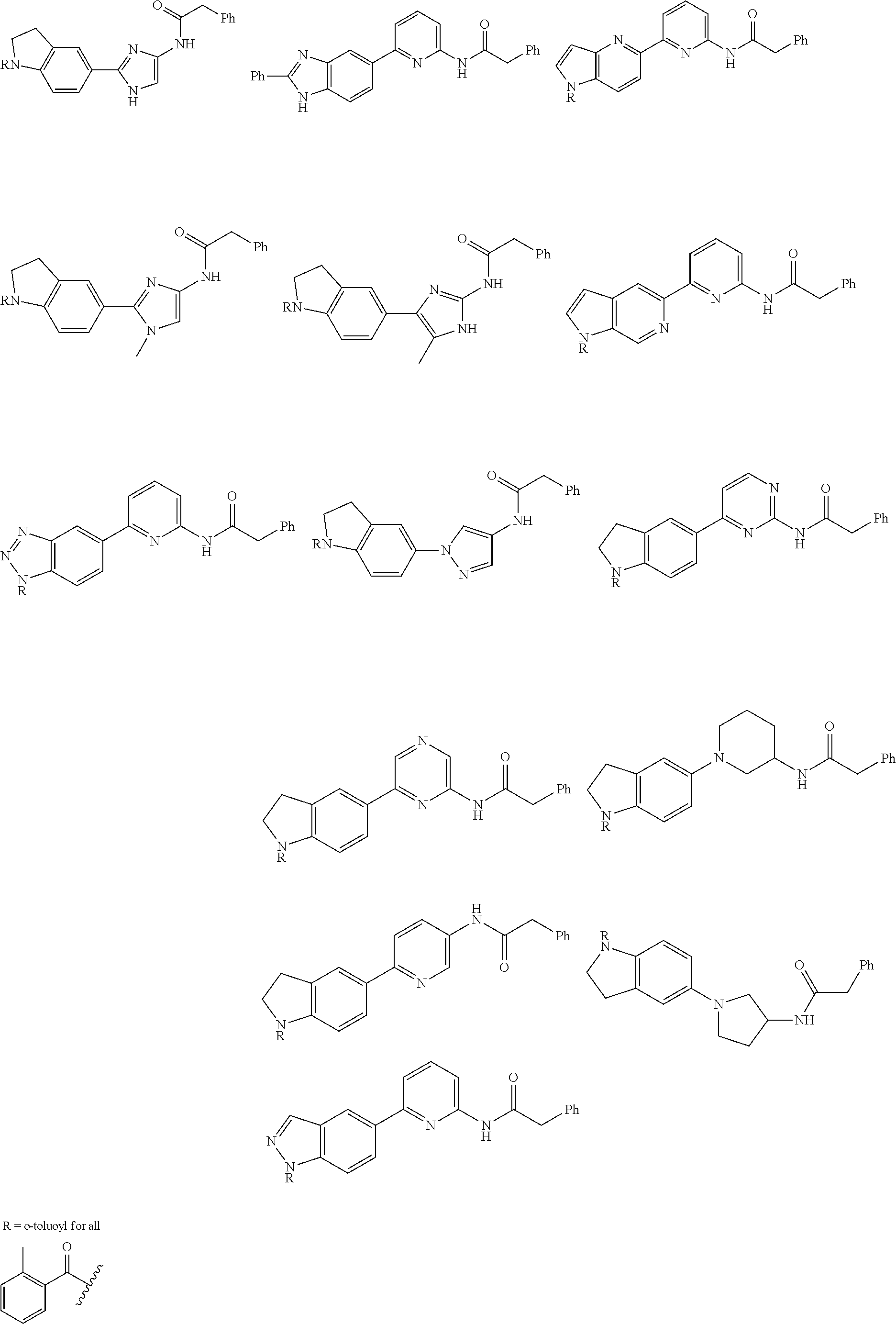

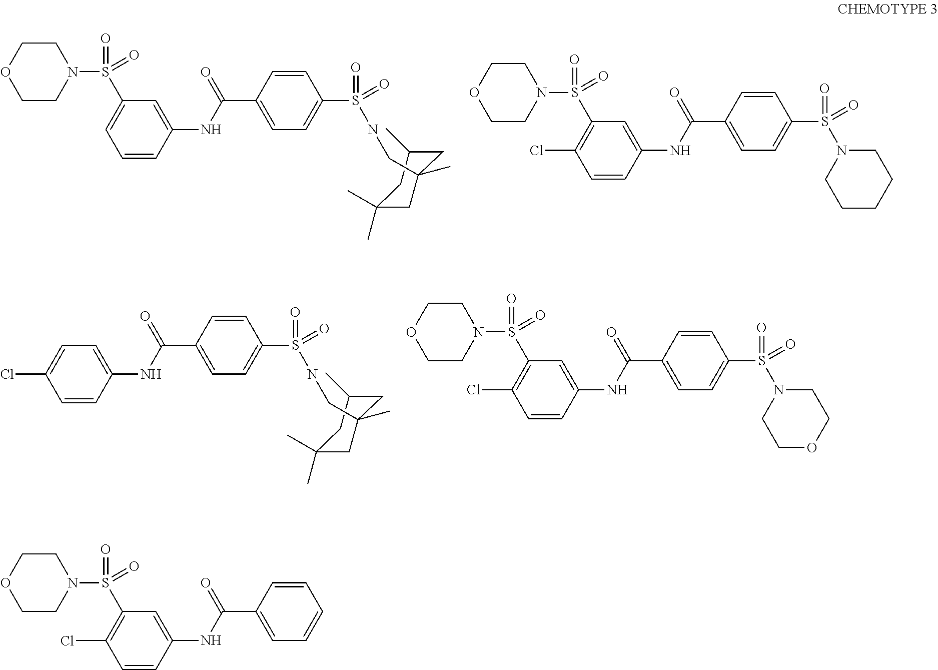 [SMILES: CC1CC(C)(C)CC(C)(CNS(=O)(=O)c2ccc(C(=O)Nc3cccc(S(=O)(=O)N4CCOCC4)c3)cc2)C1 ; O=C(Nc1ccc(Cl)c(S(=O)(=O)N2CCOCC2)c1)c1ccc(S(=O)(=O)N2CCCCC2)cc1 ; CC1CC(C)(C)CC(C)(CNS(=O)(=O)c2ccc(C(=O)Nc3ccc(Cl)cc3)cc2)C1 ; O=C(Nc1ccc(Cl)c(S(=O)(=O)N2CCOCC2)c1)c1ccc(S(=O)(=O)N2CCOCC2)cc1 ; O=C(Nc1ccc(Cl)c(S(=O)(=O)N2CCOCC2)c1)c1ccccc1]

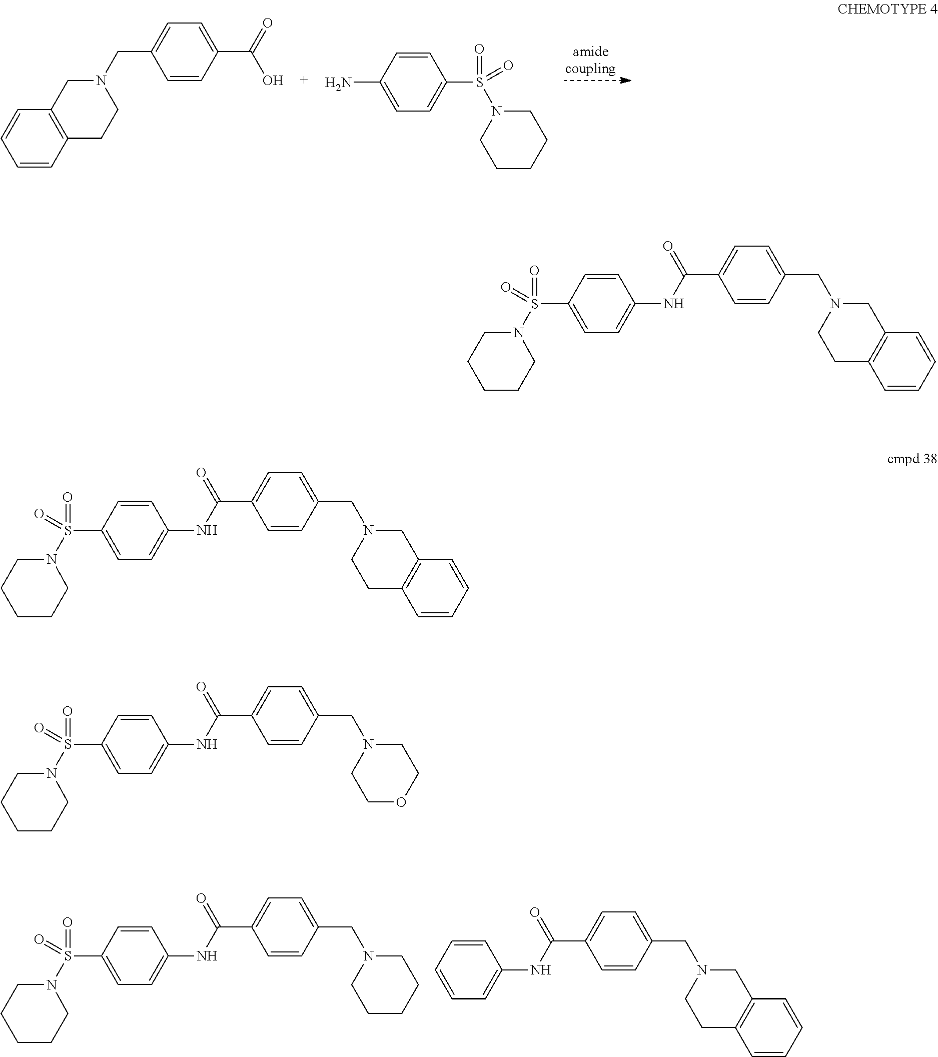

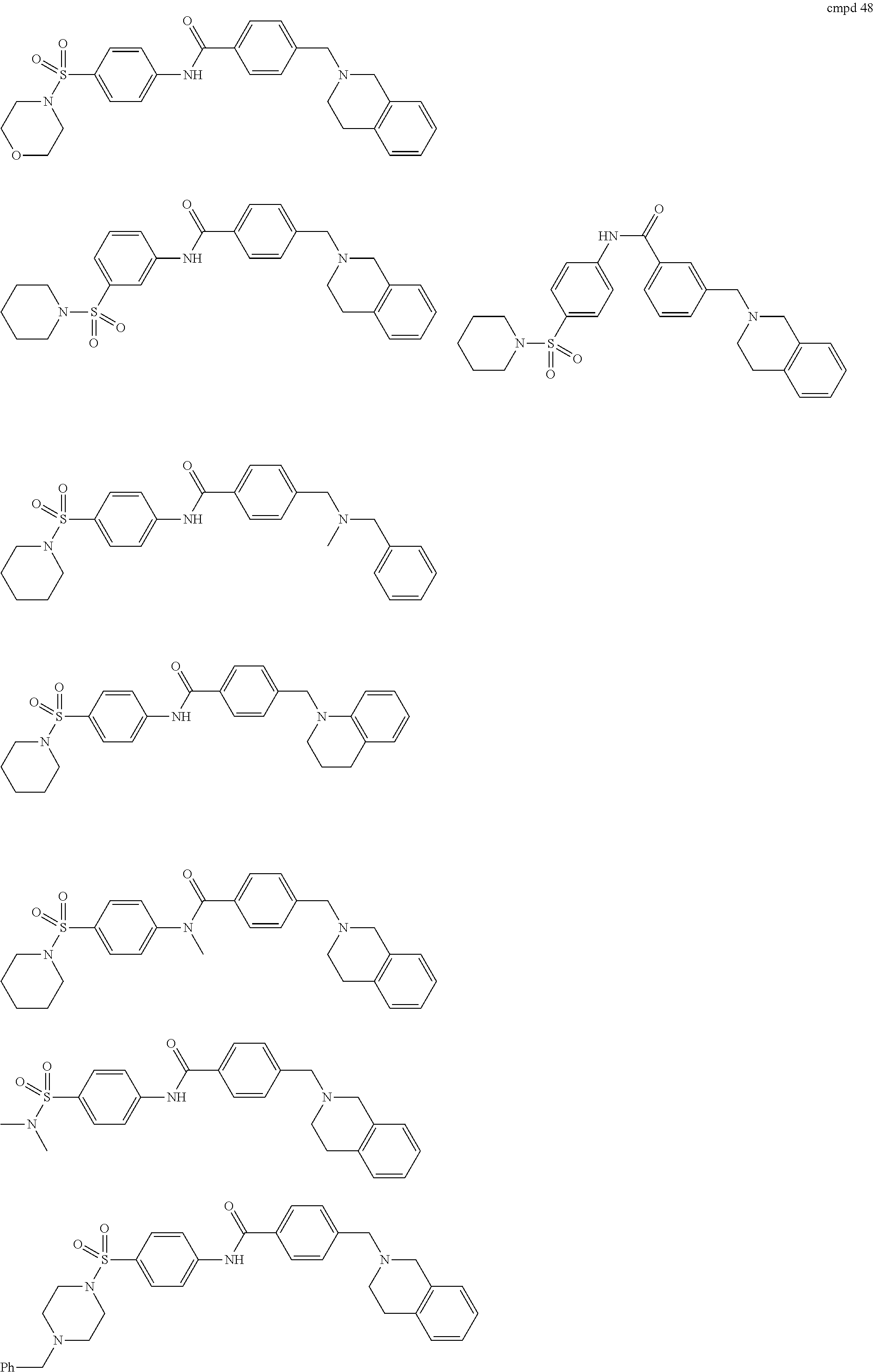

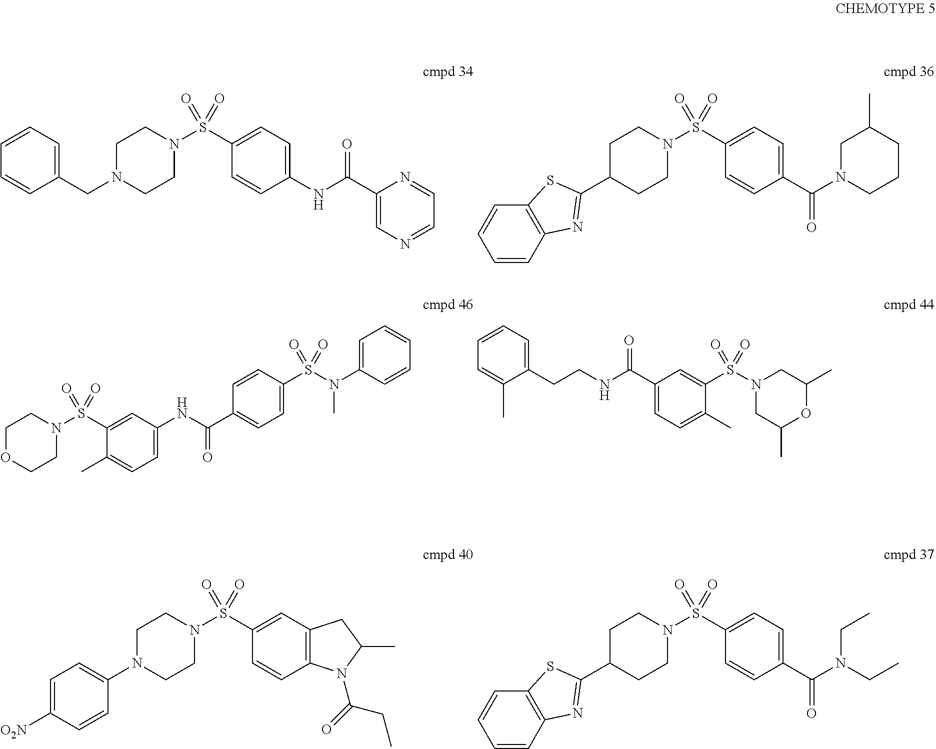

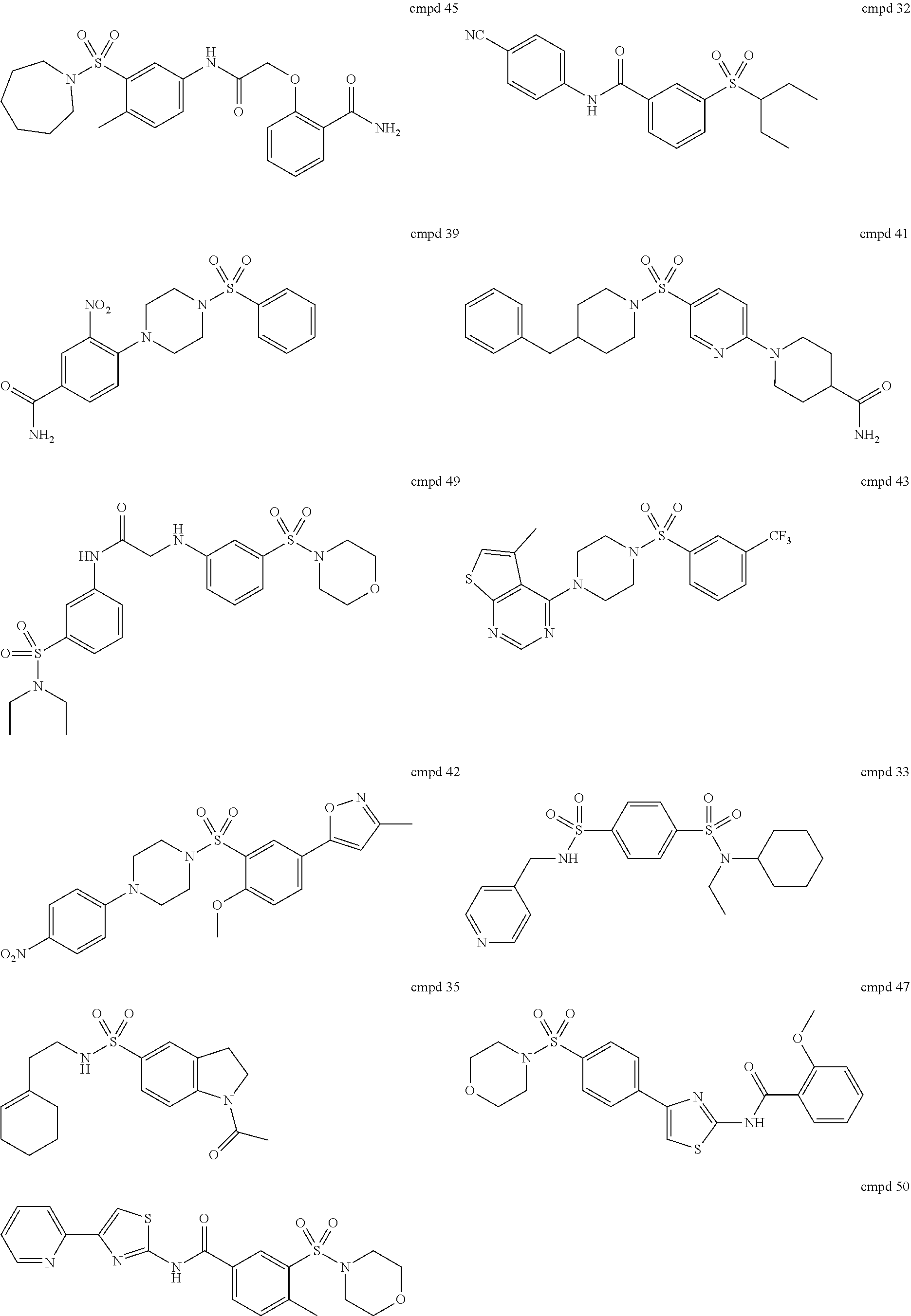

### Example 8

### Further Assays of Compounds 3, 4, and 1

Compounds 3, 4, and 1 were tested in vitro and in vivo to determine the effects of each on Nrf2 expression.

For the clonogenic assays, 1,000 cells/well were plated in a 6-well plate. The next day, the cells were treated with vehicle (DMSO), Nrf2 inhibitor, a chemotherapeutic drug, or a combination of Nrf2 and chemotherapeutic drugs for 48 hrs. The drug containing media was then replaced with regular growth media and the cells were incubated for an additional 10 days. At the end of incubation period, the colonies were stained with Crystal Violet and imaged.

For the A549 lung cancer xenograft assay, five million cells in 100 μL of PBS were implanted in the flank of nude mice. Two to three weeks post cell implantation, when the tumors were about 50 to 100 mm³ in dimension, the mice were randomly divided into four groups: vehicle, chemotherapeutic drug, Nrf2 inhibitor, and chemotherapeutic drug with an Nrf2 inhibitor. The mice were treated with the drugs for 2 to 4 weeks. Tumor volume was recorded biweekly.

For the H460 lung cancer xenograft assay, two million cells in 100 μL of PBS were implanted in the flank of nude mice. One week post cell implantation, when the tumors were about 50 to 100 mm³ in dimension, the mice were randomly divided into four groups: vehicle, chemotherapeutic drug, Nrf2 inhibitor and chemotherapeutic drug with an Nrf2 inhibitor. The mice were treated with the drugs for 2 to 4 weeks. Tumor volume was recorded biweekly.

For the in vivo pharmacokinetic studies, single intraperitoneal (IP; 30 mg/kg) and intravenous (IV; 3 mg/kg) injection was carried out using CD-1 mice. (n=3 per group/time point). Compounds in the plasma were quantified using Mass Spectrometry.

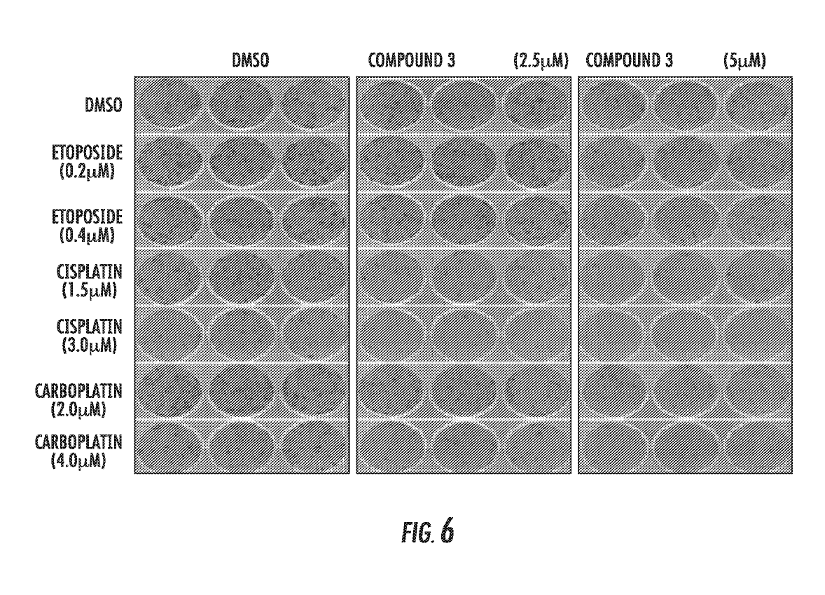

FIG. 6 shows the structure of compound 3, as well as real time PCR based validation of the ability of this compound to inhibit Nrf2 expression in A549 and H1437 cells (NC means no significant change). Inhibition of NQO1 expression as well as some inhibition of GCLm expression was also seen. In vivo pharmacokinetic studies showed that compound 3 had appropriate in vivo exposure and pharmacokinetics for in vivo use. More particularly, these studies show that compound 3 has a reasonable retention time in the blood and most of the compound is cleared from the blood in about 8 hrs.

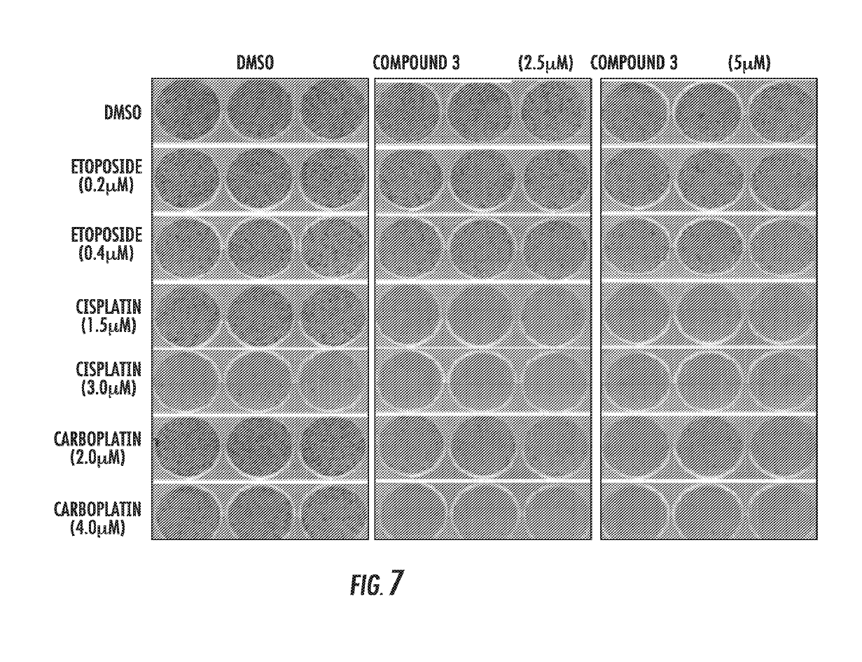

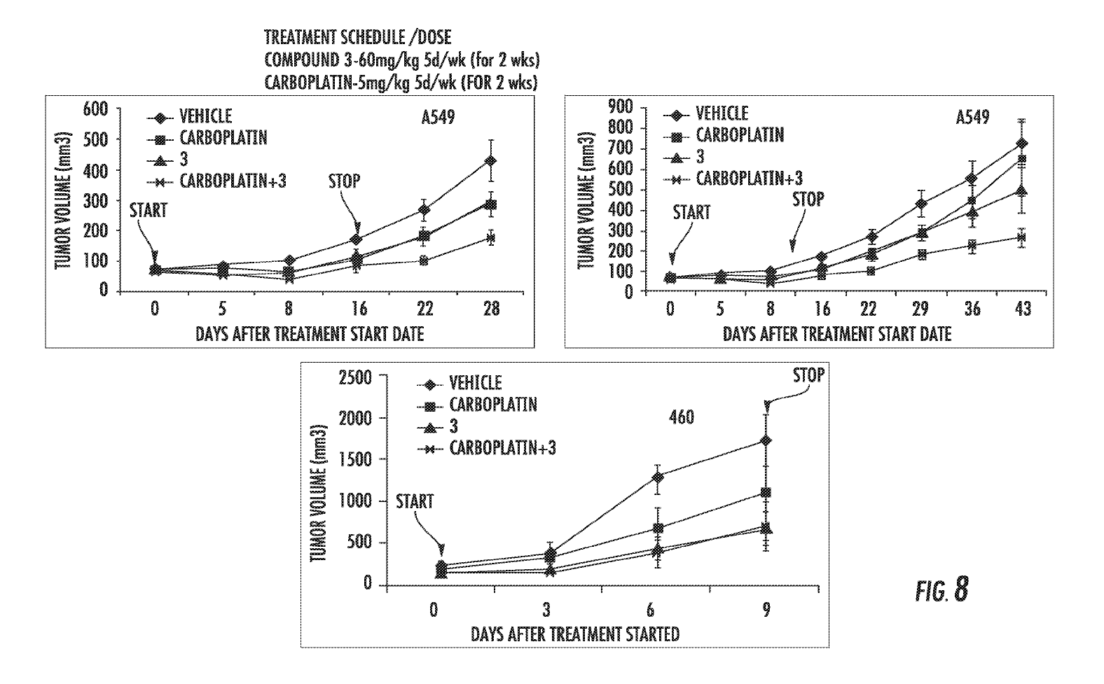

In addition, the ability of Nrf2 inhibitor 3 to enhance the cytotoxicity of standard care chemotherapeutic drugs, etoposide, cisplatin, and carboplatin, in A549 lung cancer cells (FIG. 6) and in H460 lung cancer cells (FIG. 7) was also shown. Further, xenograft assays showed that Nrf2 inhibitor 3 inhibited the growth of A549 and H460 xenograft tumors in vivo as a single agent as well as in combination with carboplatin (FIG. 8).

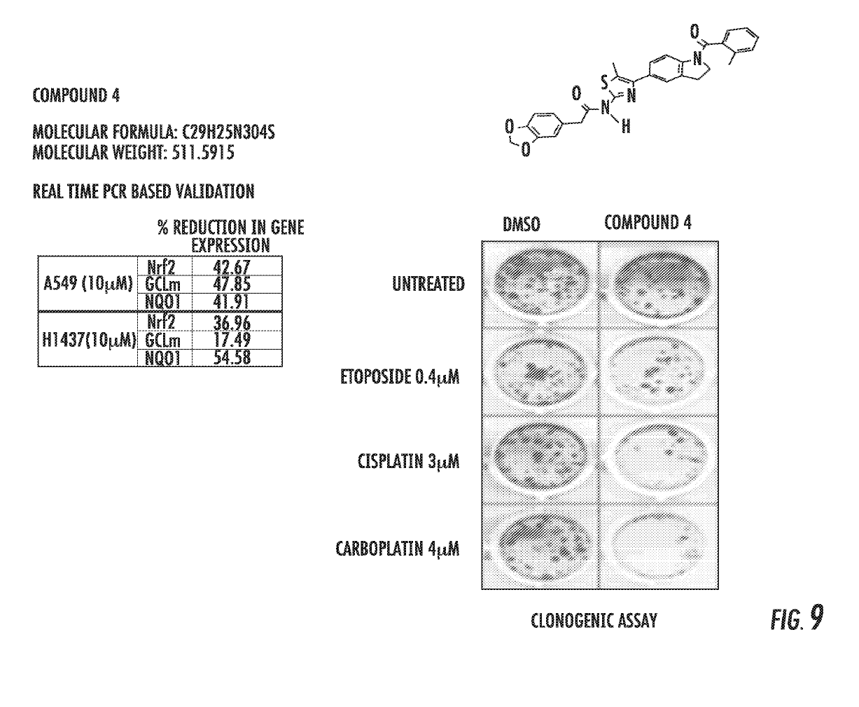

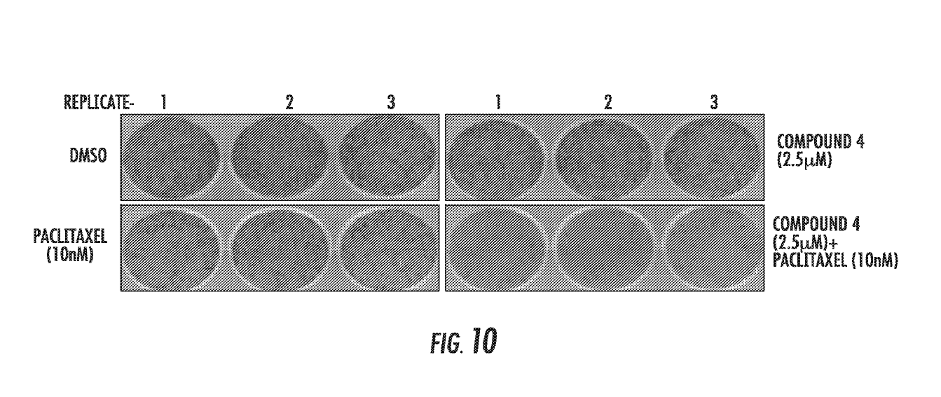

The structure of compound 4 is shown in FIG. 9. Real time PCR based validation showed a significant reduction in Nrf2, GCLm, and NQO1 expression in both A549 and H1437 cells (FIG. 9). In addition, clonogenic assays showed that this small molecule inhibitor of Nrf2 is more effective in combination with a chemotherapeutic drug (right panel of clonogenic assay in FIG. 9) in killing cancer cells compared to the chemotherapeutic drug alone (left panel of clonogenic assay in FIG. 9). The ability of this compound to enhance the cytotoxicity of the chemotherapeutic drug paclitaxel in H460 lung cancer cells also was shown (FIG. 10).

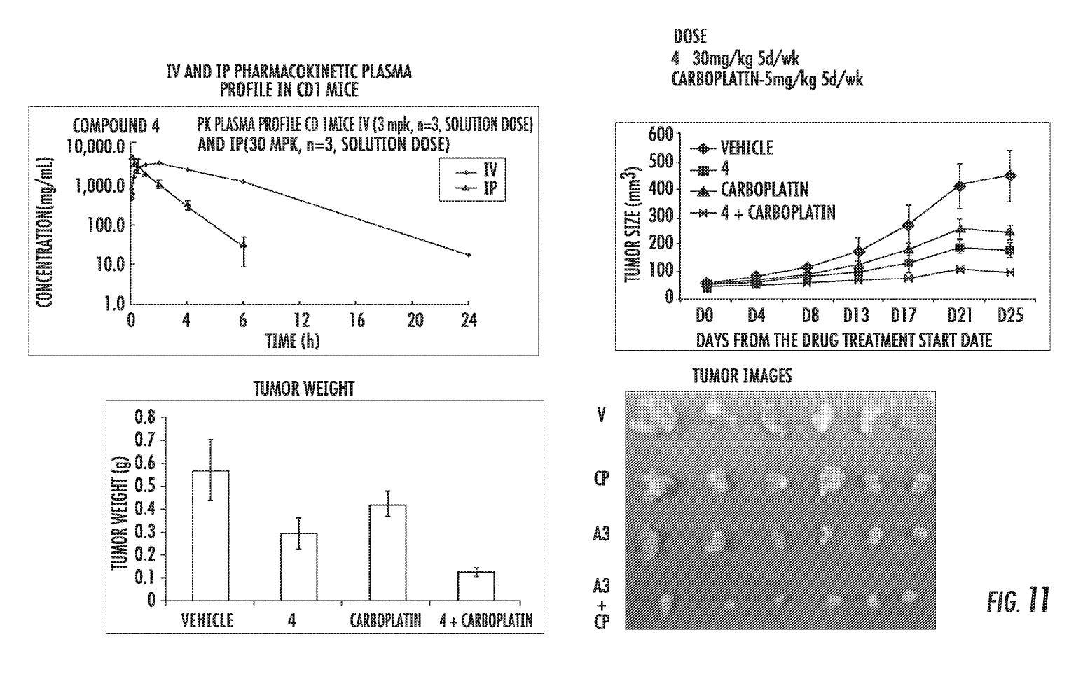

In vivo assays using compound 4 were also performed. The pharmacokinetic plasma profile of compound 4 in CD1 mice showed that its in vivo exposure and pharmacokinetics were appropriate for in vivo use (FIG. 11). More particularly, these data show that compound 4 is retained in the blood until about 12 hr, but most of the compound is cleared from the blood by 24 hrs.

Xenograft assays in vivo with compound 4 as a single agent, as well as in combination with the chemotherapeutic drug carboplatin, showed inhibition of A549 xenograft tumors. With either compound 4 alone or in combination with carboplatin, tumor size and tumor weight was reduced (FIG. 11; tumor images from top to bottom of tumor image panel: vehicle, carboplatin, compound 4, and carboplatin in combination with compound 4).

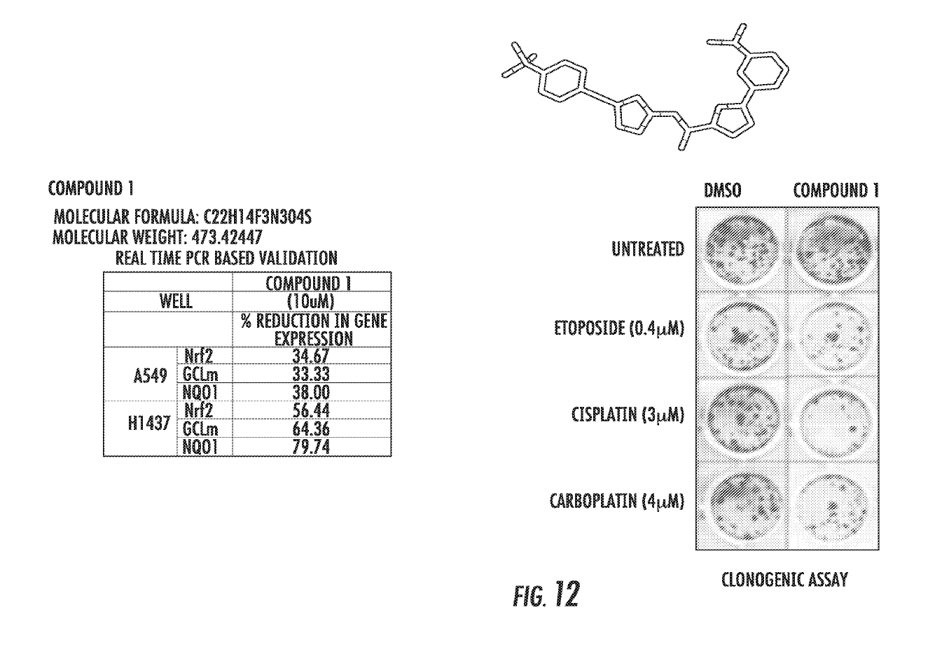

When tested in the real time PCR based validation assay, compound 1 also showed a decrease in Nrf2, GCLm, and NQO1 gene expression (FIG. 12). As seen with inhibitors 3 and 4, clonogenic assays using compound 1 showed that compound 1 also was more effective in combination with a chemotherapeutic drug (right panel of clonogenic assay in FIG. 12) in killing cancer cells compared to the chemotherapeutic drug alone (left panel of clonogenic assay in FIG. 12).

The in vivo pharmacokinetic studies using compound 1 showed that its in vivo exposure and pharmacokinetics were appropriate for in vivo use. More particularly, these data indicate that a significant amount of compound 1 is retained in the blood until about 24 hr and thus has very good drug like properties.

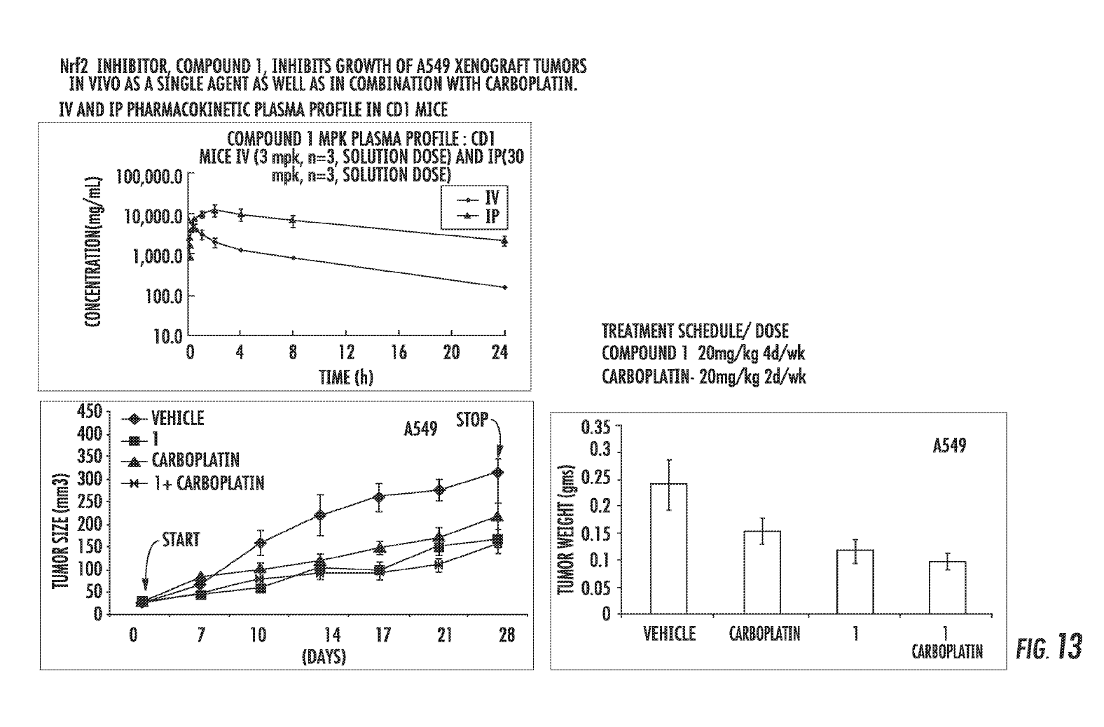

Xenograft assays in vivo with compound 1 as a single agent as well as in combination with the chemotherapeutic drug carboplatin showed inhibition of A549 xenograft tumors (FIG. 13).

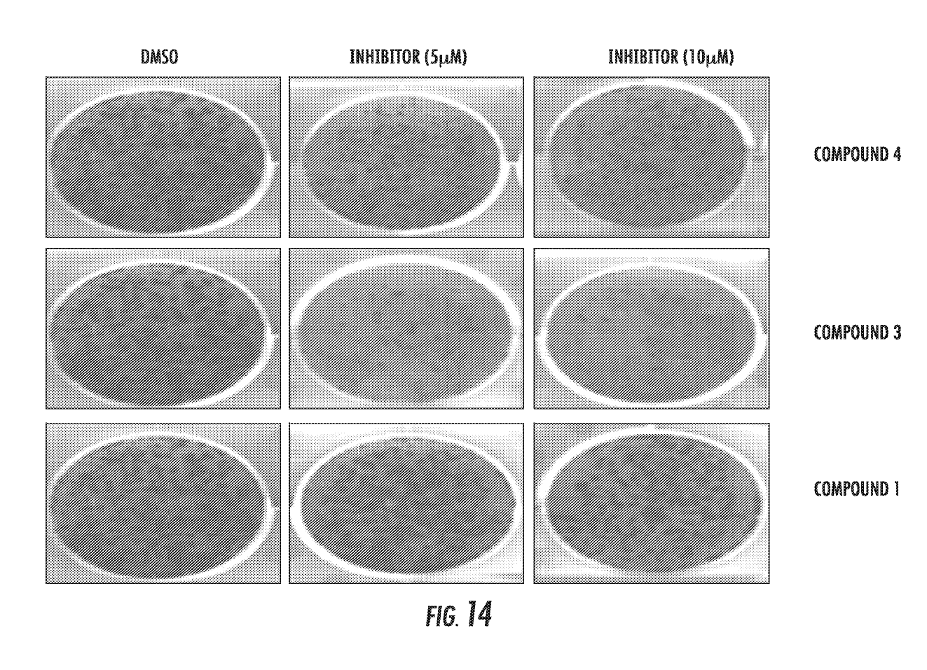

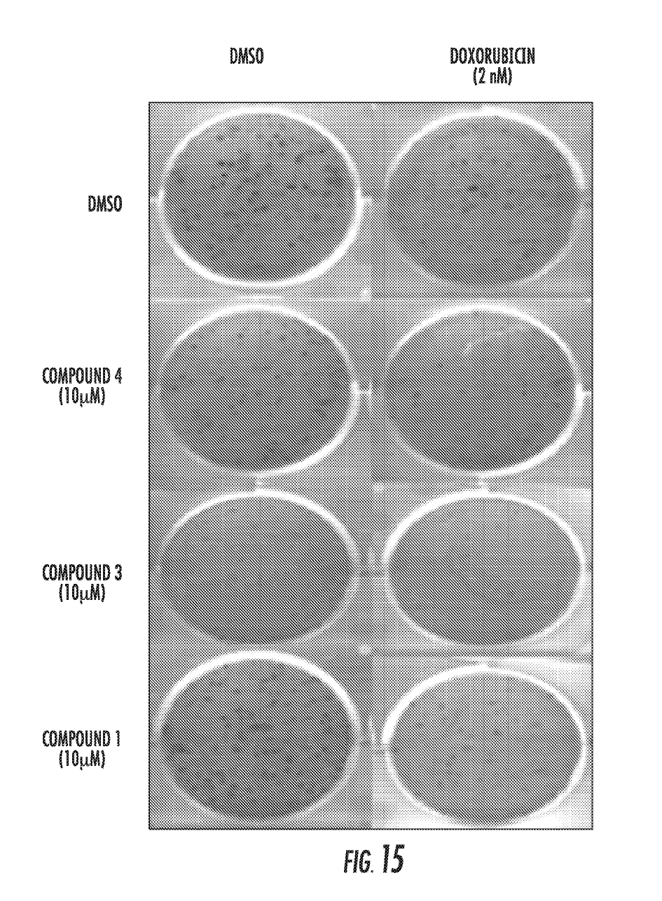

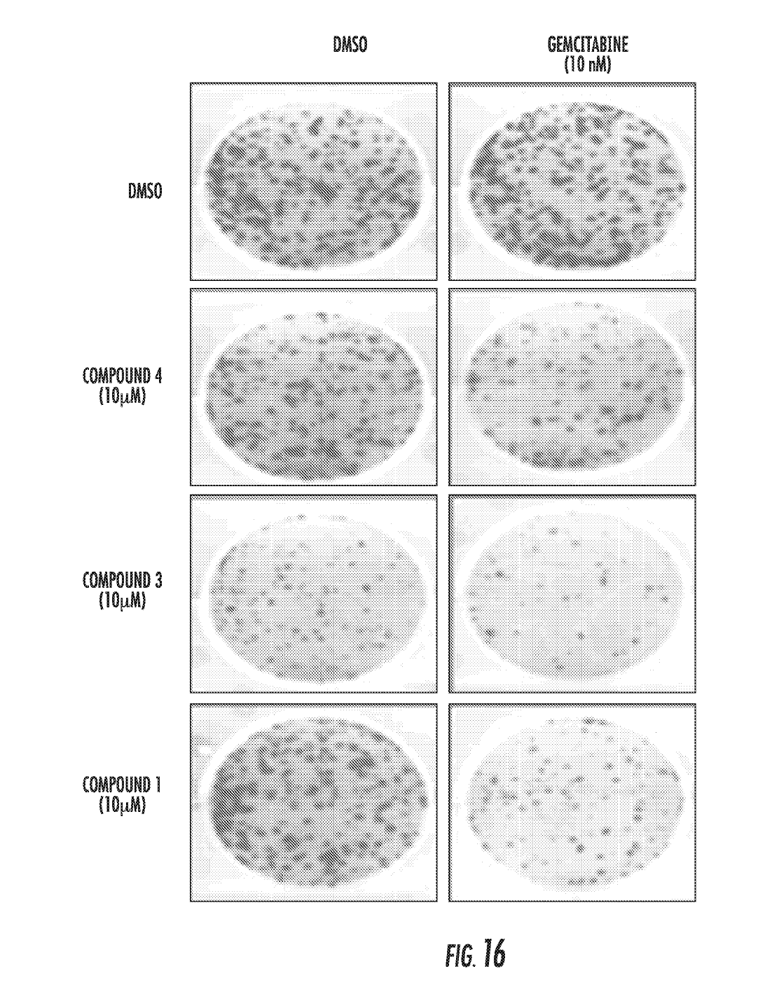

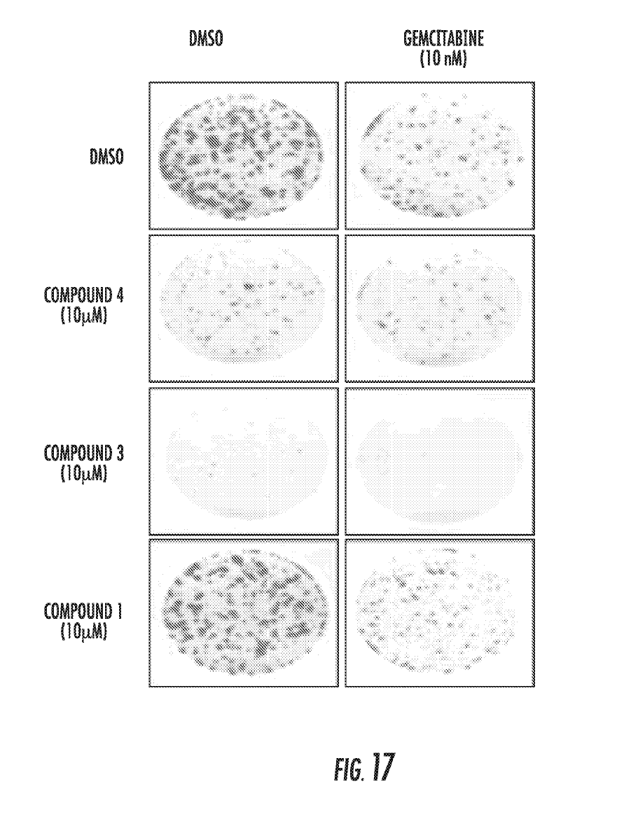

The compounds 3, 4, and 1 also were shown to suppress growth of rhabdomyosarcoma cells as single agents in the clonogenic assay (FIG. 14). In addition, these three compounds suppressed the growth of osteosarcoma cells, both as single agents as well as in combination with the chemotherapeutic drug doxorubicin (FIG. 15). Further, these compounds suppressed the growth of two different pancreatic cell lines, Panel 1 (FIG. 16) and MiaPaCa (FIG. 17), both as a single agent as well as in combination with the chemotherapeutic drug gemcitabine to enhance the cytotoxicity of gemcitabine.

The in vitro and in vivo assays using the compounds 3, 4, and 1 show that the presently disclosed compounds are effective at decreasing Nrf2 expression and can be used to treat or prevent a disease, disorder, or condition associated with an Nrf2-regulated pathway. In addition, these results demonstrate that the presently disclosed compounds can be used in combination with a chemotherapeutic drug and/or radiation therapy to render the chemotherapeutic drug and/or radiation therapy more effective.

Further, the in vivo pharmakokinetics data for compounds 3, 4, and 1 suggest that all three compounds have fairly good retention time in the blood with compound 3 being cleared the fastest and compound 1 being retained in the blood for the longest time.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Chartoumpekis, D.; Ziros, P. G.; Psyrogiannis, A; Kyriazopoulou, V.; Papavassiliou, A. G.; Habeos, I. G. Simvastatin lowers reactive oxygen species level by Nrf2 activation via P13K/Akt pathway. Biochem. Biophys. Res. Commun. 2010, 396, 463-466.
Cheng, S. E.; Lee, I. T.; Lin, C. C.; Kou, Y. R.; Yang, C. M. Cigarette smoke particle-phase extract induces HO-1 expression in human tracheal smooth muscle cells: role of the c-Src/NADPH oxidase/MAPK/Nrf2 signaling pathway. Free Radical Bioi. Med. 2010, 48, 1410-1422.
Cullinan, S. B.; Diehl, J. A. PERK-dependent activation of Nrf2 contributes to redox homeostasis and cell survival following endoplasmic reticulum stress. J. Biol. Chem. 2004, 279, 20108-20117.
Kensler, T. W.; Wakabayash, N.; Biswal, S. Cell survival responses to environmental stresses via the Keap1-Nrf2-ARE pathway. Annu. Rev. Pharmacool. Toxicol. 2007, 47, 89-116.
Kumar, S.; Singh, B. K.; Pandey, A K.; Kumar, A; Sharma, S. K; Raj, H. G.; Prasad, A. K.; Van der Eycken, E.; Parmar, V. S.; Ghosh, B. A chromone analog inhibits TNF-alpha induced expression of cell adhesion molecules on human endothelial cells via blocking NF-kappaB activation. Bioorg. Mad. Chem. 2007, 15, 2952-2962.
Merchant, A A, Singh, A, Matsui, W, Biswal, S, The redox-sensitive transcription factor Nrf2 regulates murine hematopoietic stem cell survival independently of ROS levels, Blood 2011; 118(25):6572-6579.
Misra, V; Lee, H.; Singh, A; Huang, K; Thimmulappa, R. K; Mitzner, W.; Biswal, S.; Tankersley, C. G. Global expression profiles from C57BL/6J and DBA/2J mouse lungs to determine aging-related genes. Physiological Genomics. 2007, 31, 429-440.
Nioi, P.; McMahon, M.; Itoh, K.; Yamamoto, M.; Hayes, J. D. Identification of a novel Nrf2-regulated antioxidant response element (ARE) in the mouse NAD(P)H: quinone oxidoreductase 1 gene: reassessment of the ARE consensus sequence. Biochem. J. 2003, 374, 337-348.
Niture, S. K.; Jain, A. K; Jaiswal, A. K. Antioxidant-induced modification of INrf2 cysteine 151 and PKC-delta-mediated phosphorylation of Nrf2 serine 40 are both required for stabilization and nuclear translocation of Nrf2 and increased drug resistance. J. Cell Sci. 2009, 122, 4452-4464.
Rangasamy, T.; Guo, J.; Mitzner, W. A.; Roman, J.; Singh, A.; Fryer, A. D.; Yamamoto, M.; Kensler, T. W.; Tuder, R. M.; Georas, S. N.; Biswal, S. Disruption of Nrf2 enhances susceptibility to severe airway inflammation and asthma in mice. J. Exp. Med. 2005, 202, 47-59.
Singh, A; Boldin-Adamsky, S.; Thimmulappa, R. K; Rath, S. K.; Ashush, H; Coulter, J.; Blackford, A; Goodman, S. N.; Bunz, F.; Watson, W. H.; Gabrielson, E.; Feinstein, E.; Biswal, S. RNAi-mediated silencing of nuclear factor erythroid-2-related factor 2 gene expression in non-small cell lung cancer inhibits tumor growth and increases efficacy of chemotherapy. Cancer Res. 2008, 68, 7975-7984.
Surh, Y. J.; Kundu, J. K.; Na, H. K. Nrf2 as a Master Redox Switch in Turning on the Cellular Signaling Involved in the Induction of Cytoprotective Genes by Some Chemopreventive Phytochemicals. Planta Med. 2008, 74, 1526-1539.
Sussan, T. E.; Rangasamy, T.; Blake, D. J.; Malhotra, D.; EI-Haddad, H.; Bedja, D.; Yates, M. S.; Kombairaju, P.; Yamamoto, M.; Liby, K. T.; Sporn, M. B.; Gabrielson, K. I.; Champion, H. C.; Tuder, R. M.; Kensler, T. W.; Biswal, S. Targeting Nrf2 with the triterpenoid CDDO-imidazolide attenuates cigarette smoke-induced emphysema and cardiac dysfunction in mice. Proc. Natl. Acad. Sci. U.S.A 2009, 106, 250-255.
Kumar, S.; Singh, B. K.; Pandey, A K.; Kumar, A; Sharma, S. K; Raj, H. G.; Prasad, A. K.; Van der Eycken, E.; Parmar, V. S.; Ghosh, B. A chromone analog inhibits TNF-alpha induced expression of cell adhesion molecules on human endothelial cells via blocking NF-kappaB activation. Bioorg. Mad. Chem. 2007, 15, 2952-2962.

Although the foregoing subject matter has been described in some detail by way of illustration and example for purposes of clarity of understanding, it will be understood by those skilled in the art that certain changes and modifications can be practiced within the scope of the appended claims.

1. A compound selected from the group consisting of: 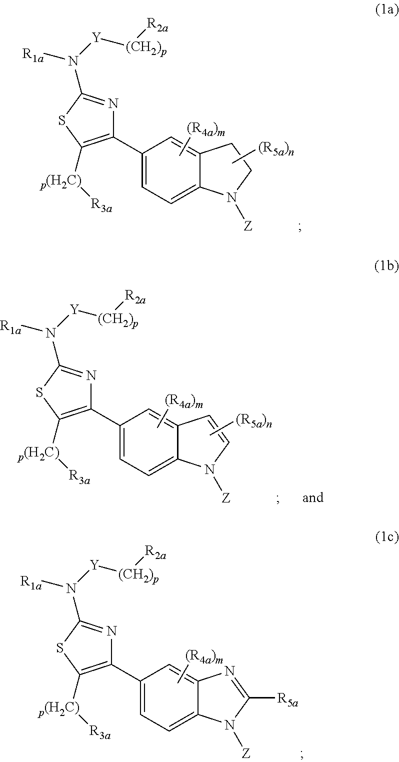
wherein:
m is an integer selected from the group consisting of 0, 1, 2, and 3;
n is an integer selected from the group consisting of 0, 1, and 2;
each p is independently an integer selected from the group consisting of 0, 1, and 2;
R₁ₐ is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;
R₂ₐ is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;
R₃ₐ is selected from the group consisting of H and substituted or unsubstituted straight-chain or branched alkyl;
each R₄ₐ and R₅ₐ is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;
R₆ₐ is selected from the group consisting of H, substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;
Y is —(C═O)— or —S(═O)₂—; and
Z is selected from the group consisting of R₆ₐ, —C(═O)—(CH₂)ₚ—R₆ₐ, and —S(═O)₂—R₆ₐ; p is an integer selected from the group consisting of 0, 1, and 2;
or an enantiomer, diastereomer, racemate or pharmaceutically acceptable salt, prodrug, or solvate thereof;
under the proviso that the compound of formula (1a) is not a compound selected from the group consisting of:

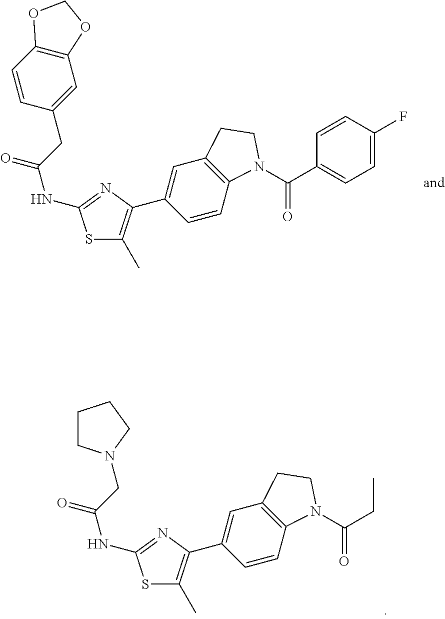 [SMILES: Cc1sc(NC(=O)Cc2ccc3c(c2)OCO3)nc1-c1ccc2c(c1)CCN2C(=O)c1ccc(F)cc1 ; CCC(=O)N1CCc2cc(-c3nc(NC(=O)CN4CCCC4)sc3C)ccc21]

2. The compound of claim 1, wherein the compound is a compound of Formula (1a′):

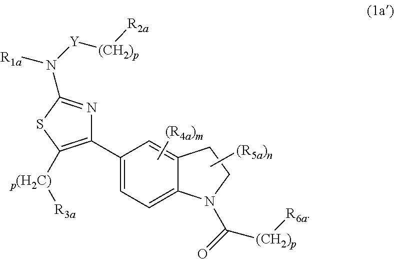

3. The compound of claim 2, wherein the compound of formula (1a′) is selected from the group consisting of:

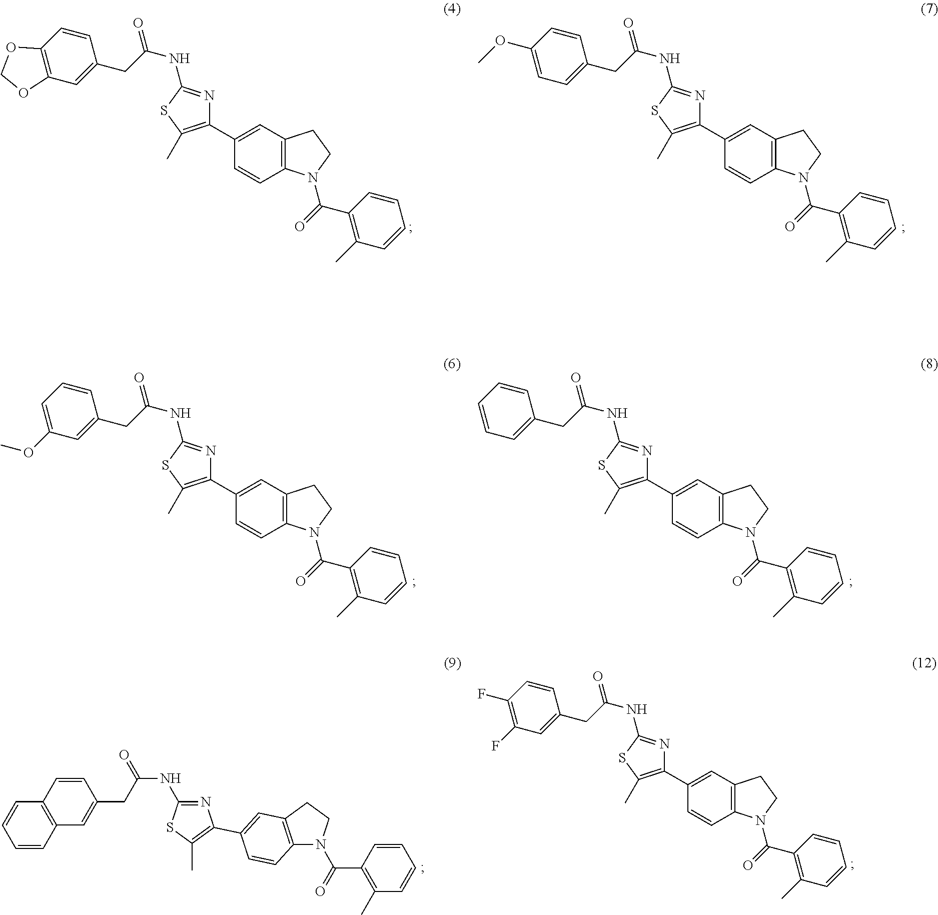 [SMILES: Cc1ccccc1C(=O)N1CCc2cc(-c3nc(NC(=O)Cc4ccc5c(c4)OCO5)sc3C)ccc21 ; COc1ccc(CC(=O)Nc2nc(-c3ccc4c(c3)CCN4C(=O)c3ccccc3C)c(C)s2)cc1 ; COc1cccc(CC(=O)Nc2nc(-c3ccc4c(c3)CCN4C(=O)c3ccccc3C)c(C)s2)c1 ; Cc1ccccc1C(=O)N1CCc2cc(-c3nc(NC(=O)Cc4ccccc4)sc3C)ccc21 ; Cc1ccccc1C(=O)N1CCc2cc(-c3nc(NC(=O)Cc4ccc5ccccc5c4)sc3C)ccc21 ; Cc1ccccc1C(=O)N1CCc2cc(-c3nc(NC(=O)Cc4ccc(F)c(F)c4)sc3C)ccc21]

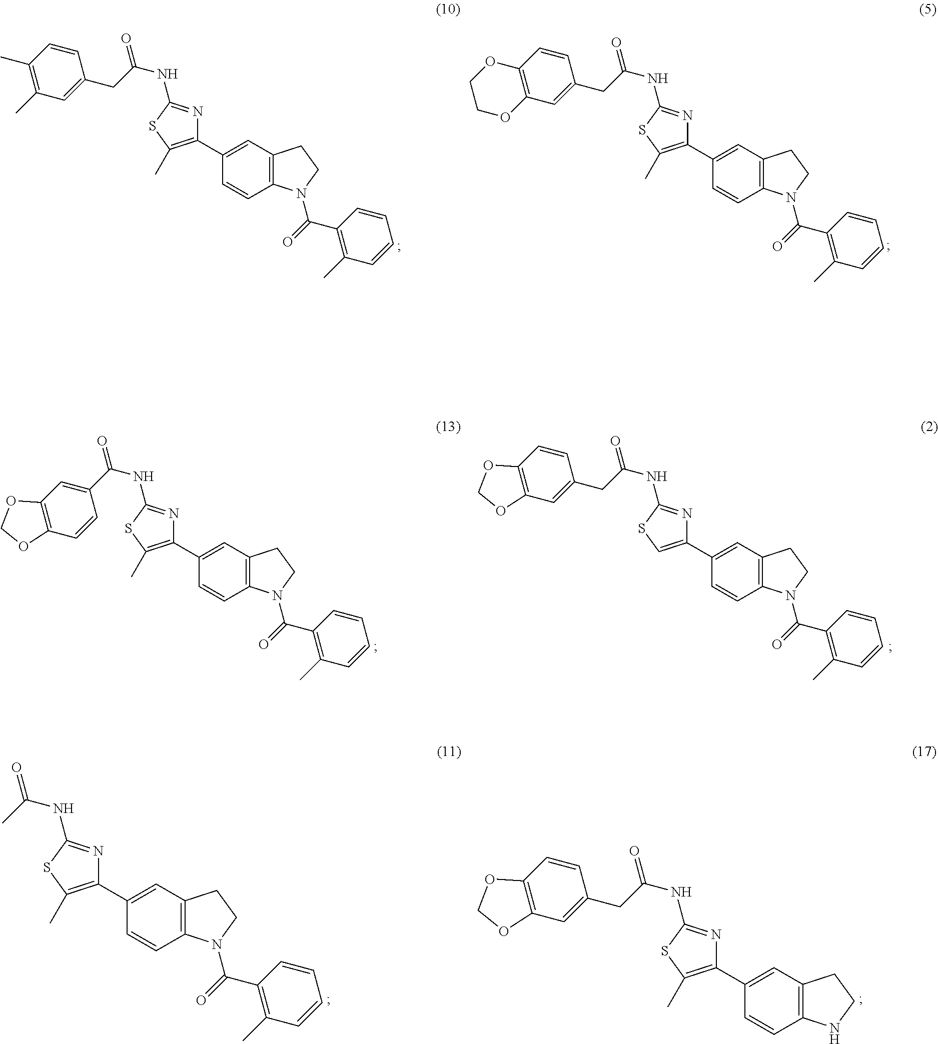 [SMILES: Cc1ccc(CC(=O)Nc2nc(-c3ccc4c(c3)CCN4C(=O)c3ccccc3C)c(C)s2)cc1C ; Cc1ccccc1C(=O)N1CCc2cc(-c3nc(NC(=O)Cc4ccc5c(c4)OCCO5)sc3C)ccc21 ; Cc1ccccc1C(=O)N1CCc2cc(-c3nc(NC(=O)c4ccc5c(c4)OCO5)sc3C)ccc21 ; Cc1ccccc1C(=O)N1CCc2cc(-c3csc(NC(=O)Cc4ccc5c(c4)OCO5)n3)ccc21 ; CC(=O)Nc1nc(-c2ccc3c(c2)CCN3C(=O)c2ccccc2C)c(C)s1 ; Cc1sc(NC(=O)Cc2ccc3c(c2)OCO3)nc1-c1ccc2c(c1)CCN2]

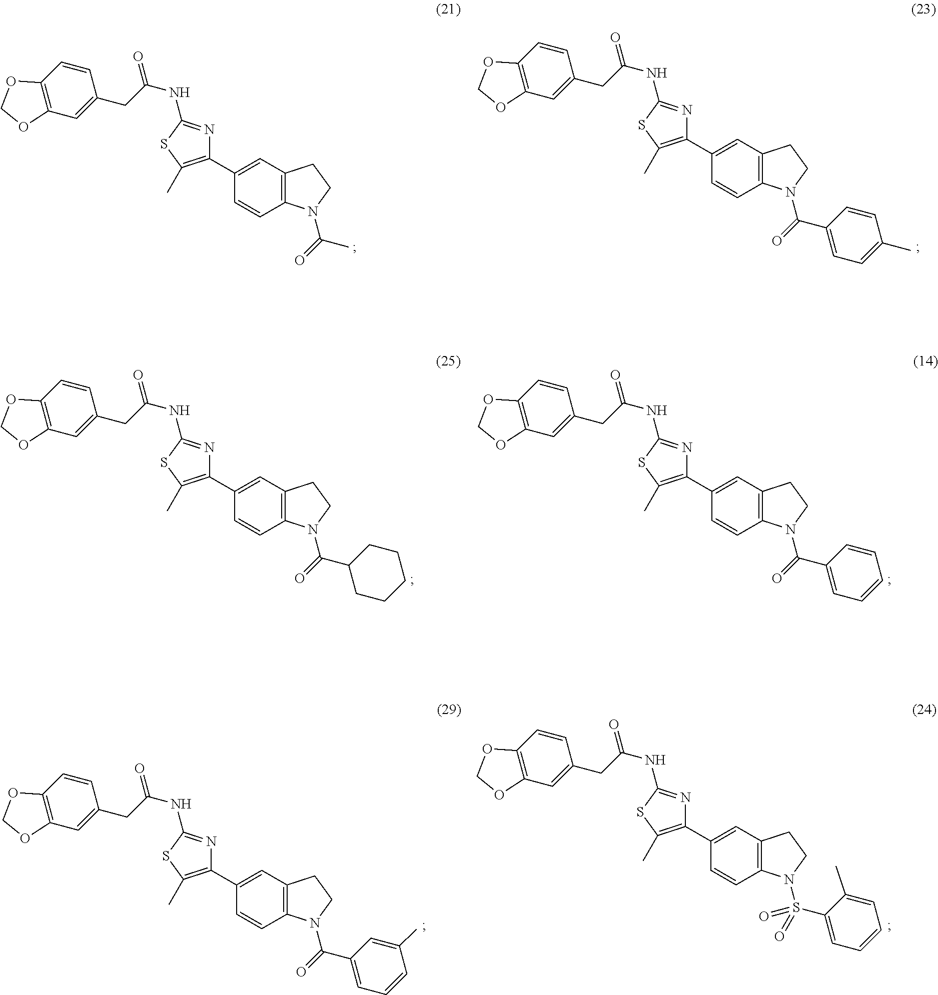 [SMILES: CC(=O)N1CCc2cc(-c3nc(NC(=O)Cc4ccc5c(c4)OCO5)sc3C)ccc21 ; Cc1ccc(C(=O)N2CCc3cc(-c4nc(NC(=O)Cc5ccc6c(c5)OCO6)sc4C)ccc32)cc1 ; Cc1sc(NC(=O)Cc2ccc3c(c2)OCO3)nc1-c1ccc2c(c1)CCN2C(=O)C1CCCCC1 ; Cc1sc(NC(=O)Cc2ccc3c(c2)OCO3)nc1-c1ccc2c(c1)CCN2C(=O)c1ccccc1 ; Cc1cccc(C(=O)N2CCc3cc(-c4nc(NC(=O)Cc5ccc6c(c5)OCO6)sc4C)ccc32)c1 ; Cc1ccccc1S(=O)(=O)N1CCc2cc(-c3nc(NC(=O)Cc4ccc5c(c4)OCO5)sc3C)ccc21]

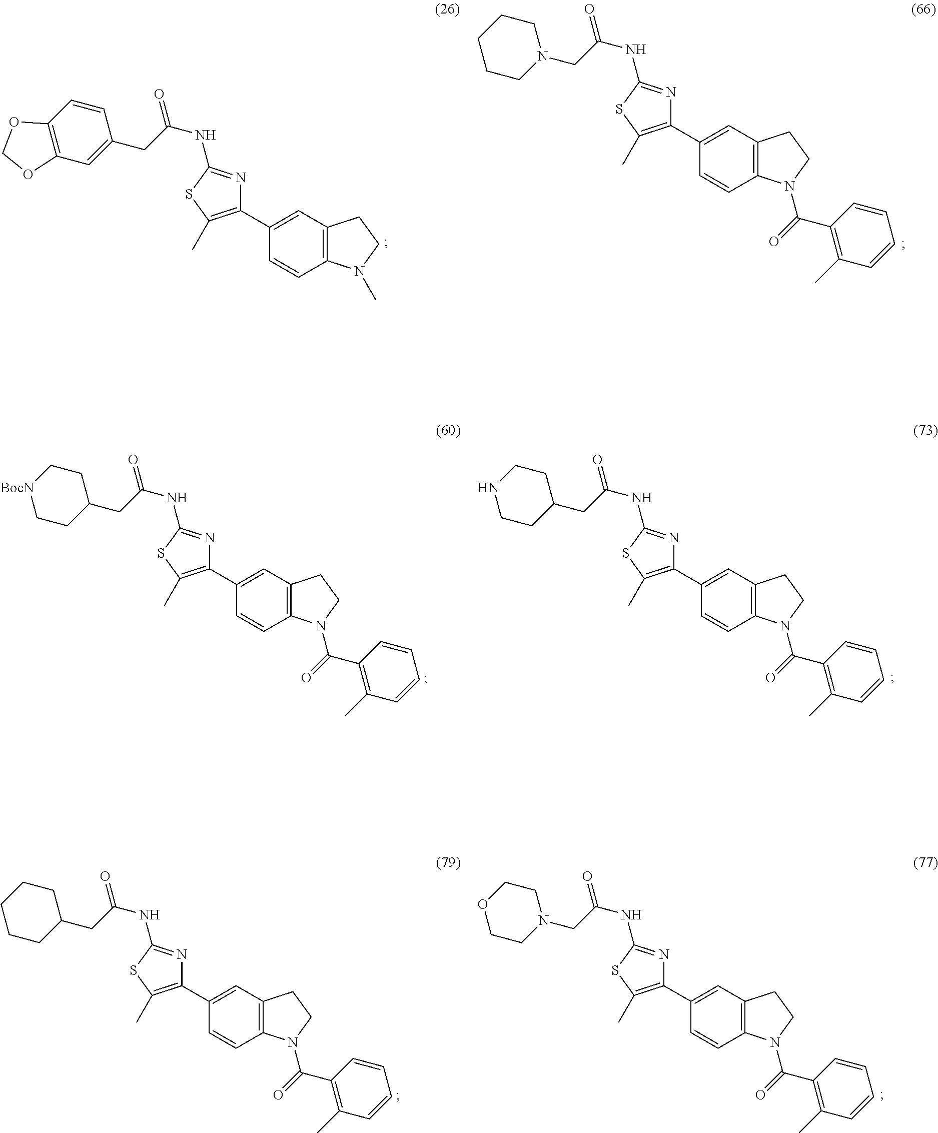 [SMILES: Cc1sc(NC(=O)Cc2ccc3c(c2)OCO3)nc1-c1ccc2c(c1)CCN2C ; Cc1ccccc1C(=O)N1CCc2cc(-c3nc(NC(=O)CN4CCCCC4)sc3C)ccc21 ; Cc1ccccc1C(=O)N1CCc2cc(-c3nc(NC(=O)CC4CCN(C(=O)OC(C)(C)C)CC4)sc3C)ccc21 ; Cc1ccccc1C(=O)N1CCc2cc(-c3nc(NC(=O)CC4CCNCC4)sc3C)ccc21 ; Cc1ccccc1C(=O)N1CCc2cc(-c3nc(NC(=O)CC4CCCCC4)sc3C)ccc21 ; Cc1ccccc1C(=O)N1CCc2cc(-c3nc(NC(=O)CN4CCOCC4)sc3C)ccc21]

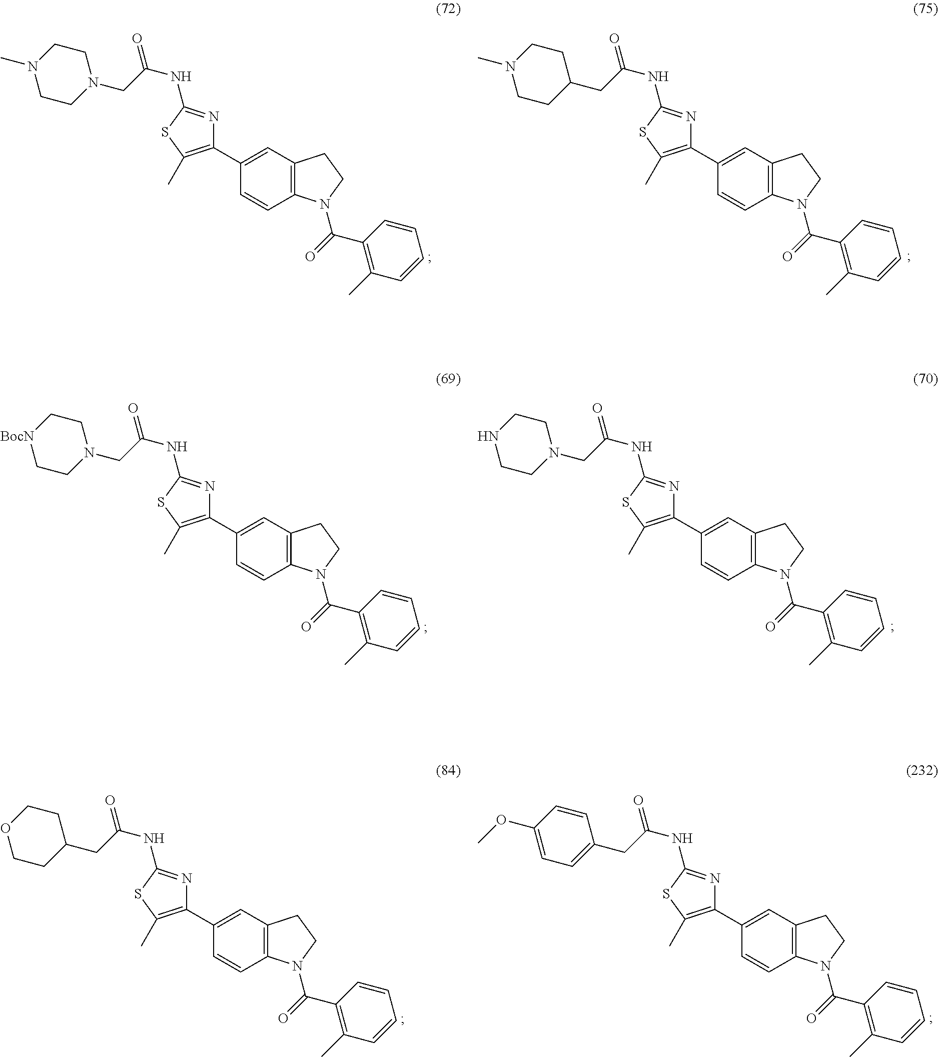 [SMILES: Cc1ccccc1C(=O)N1CCc2cc(-c3nc(NC(=O)CN4CCN(C)CC4)sc3C)ccc21 ; Cc1ccccc1C(=O)N1CCc2cc(-c3nc(NC(=O)CC4CCN(C)CC4)sc3C)ccc21 ; Cc1ccccc1C(=O)N1CCc2cc(-c3nc(NC(=O)CN4CCN(C(=O)OC(C)(C)C)CC4)sc3C)ccc21 ; Cc1ccccc1C(=O)N1CCc2cc(-c3nc(NC(=O)CN4CCNCC4)sc3C)ccc21 ; Cc1ccccc1C(=O)N1CCc2cc(-c3nc(NC(=O)CC4CCOCC4)sc3C)ccc21 ; COc1ccc(CC(=O)Nc2nc(-c3ccc4c(c3)CCN4C(=O)c3ccccc3C)c(C)s2)cc1]

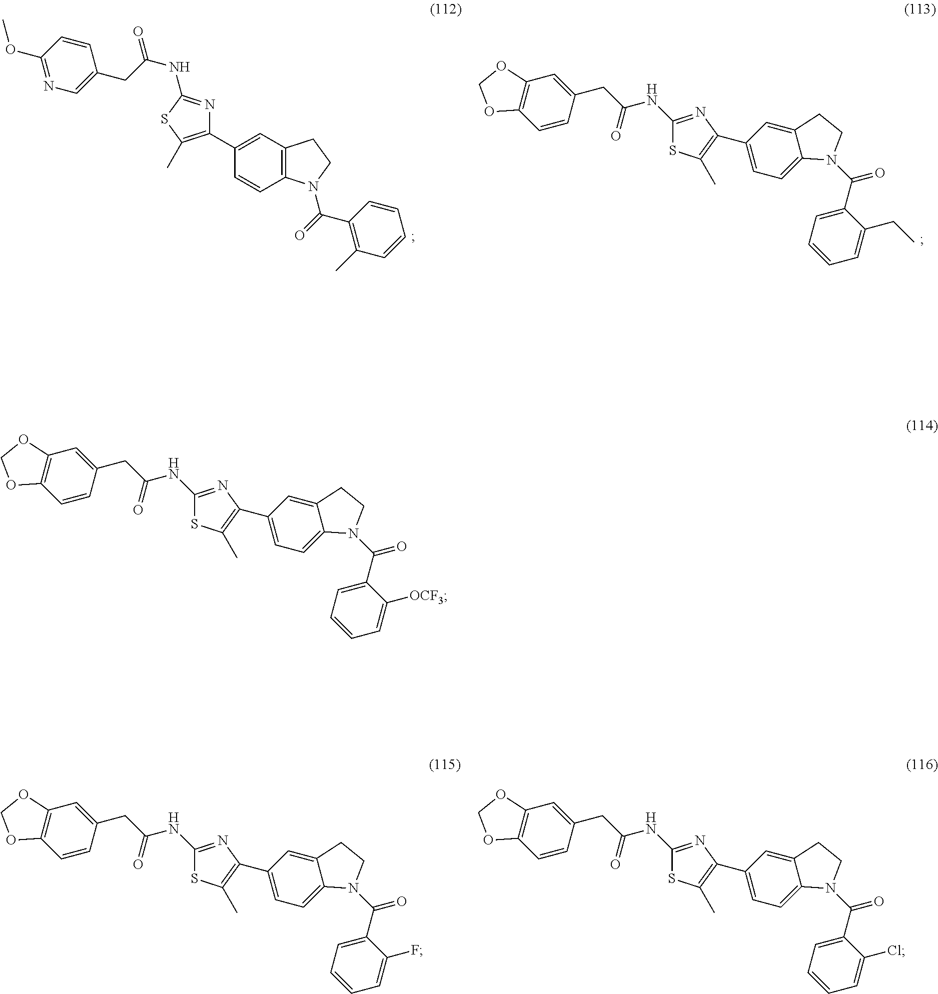 [SMILES: COc1ccc(CC(=O)Nc2nc(-c3ccc4c(c3)CCN4C(=O)c3ccccc3C)c(C)s2)cn1 ; CCc1ccccc1C(=O)N1CCc2cc(-c3nc(NC(=O)Cc4ccc5c(c4)OCO5)sc3C)ccc21 ; Cc1sc(NC(=O)Cc2ccc3c(c2)OCO3)nc1-c1ccc2c(c1)CCN2C(=O)c1ccccc1OC(F)(F)F ; Cc1sc(NC(=O)Cc2ccc3c(c2)OCO3)nc1-c1ccc2c(c1)CCN2C(=O)c1ccccc1F ; Cc1sc(NC(=O)Cc2ccc3c(c2)OCO3)nc1-c1ccc2c(c1)CCN2C(=O)c1ccccc1Cl]

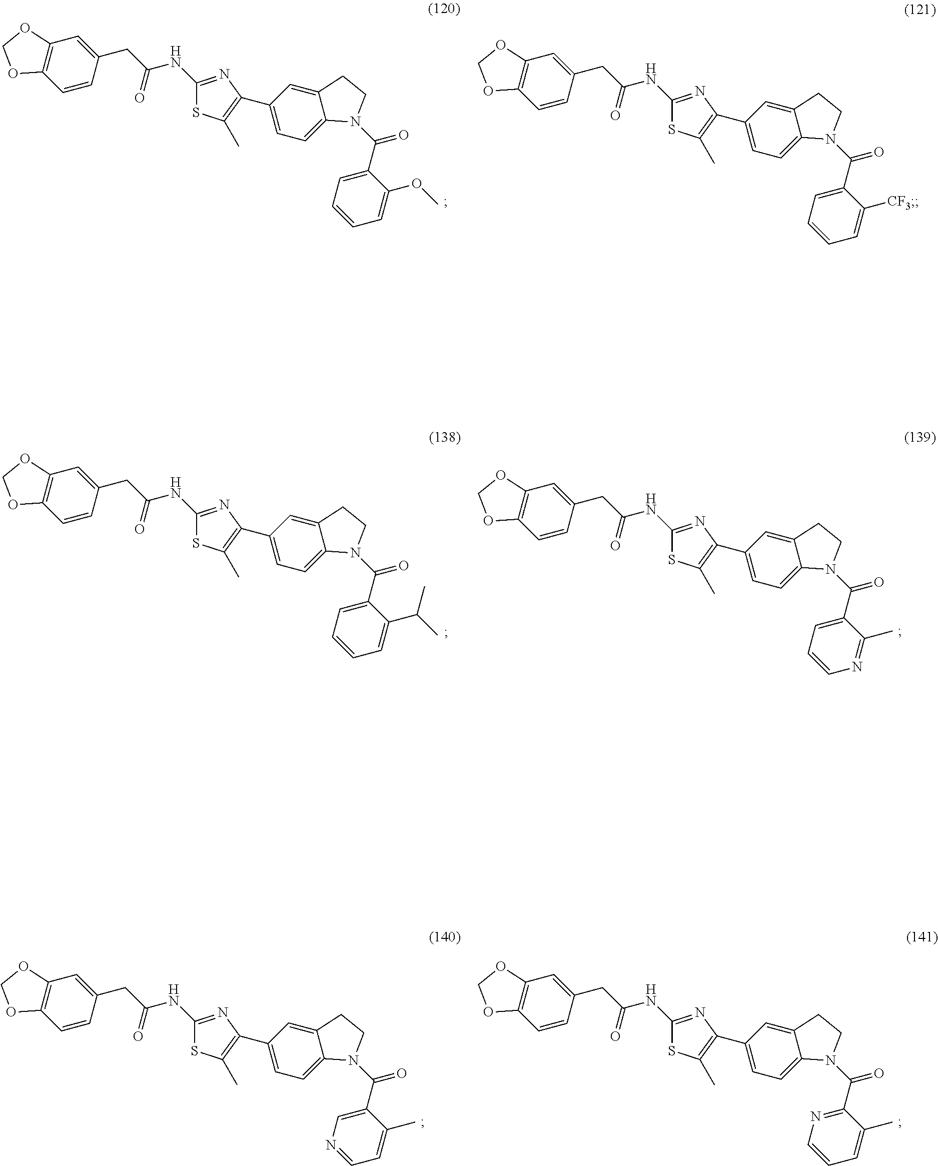 [SMILES: COc1ccccc1C(=O)N1CCc2cc(-c3nc(NC(=O)Cc4ccc5c(c4)OCO5)sc3C)ccc21 ; Cc1sc(NC(=O)Cc2ccc3c(c2)OCO3)nc1-c1ccc2c(c1)CCN2C(=O)c1ccccc1C(F)(F)F ; Cc1sc(NC(=O)Cc2ccc3c(c2)OCO3)nc1-c1ccc2c(c1)CCN2C(=O)c1ccccc1C(C)C ; Cc1ncccc1C(=O)N1CCc2cc(-c3nc(NC(=O)Cc4ccc5c(c4)OCO5)sc3C)ccc21 ; Cc1ccncc1C(=O)N1CCc2cc(-c3nc(NC(=O)Cc4ccc5c(c4)OCO5)sc3C)ccc21 ; Cc1cccnc1C(=O)N1CCc2cc(-c3nc(NC(=O)Cc4ccc5c(c4)OCO5)sc3C)ccc21]

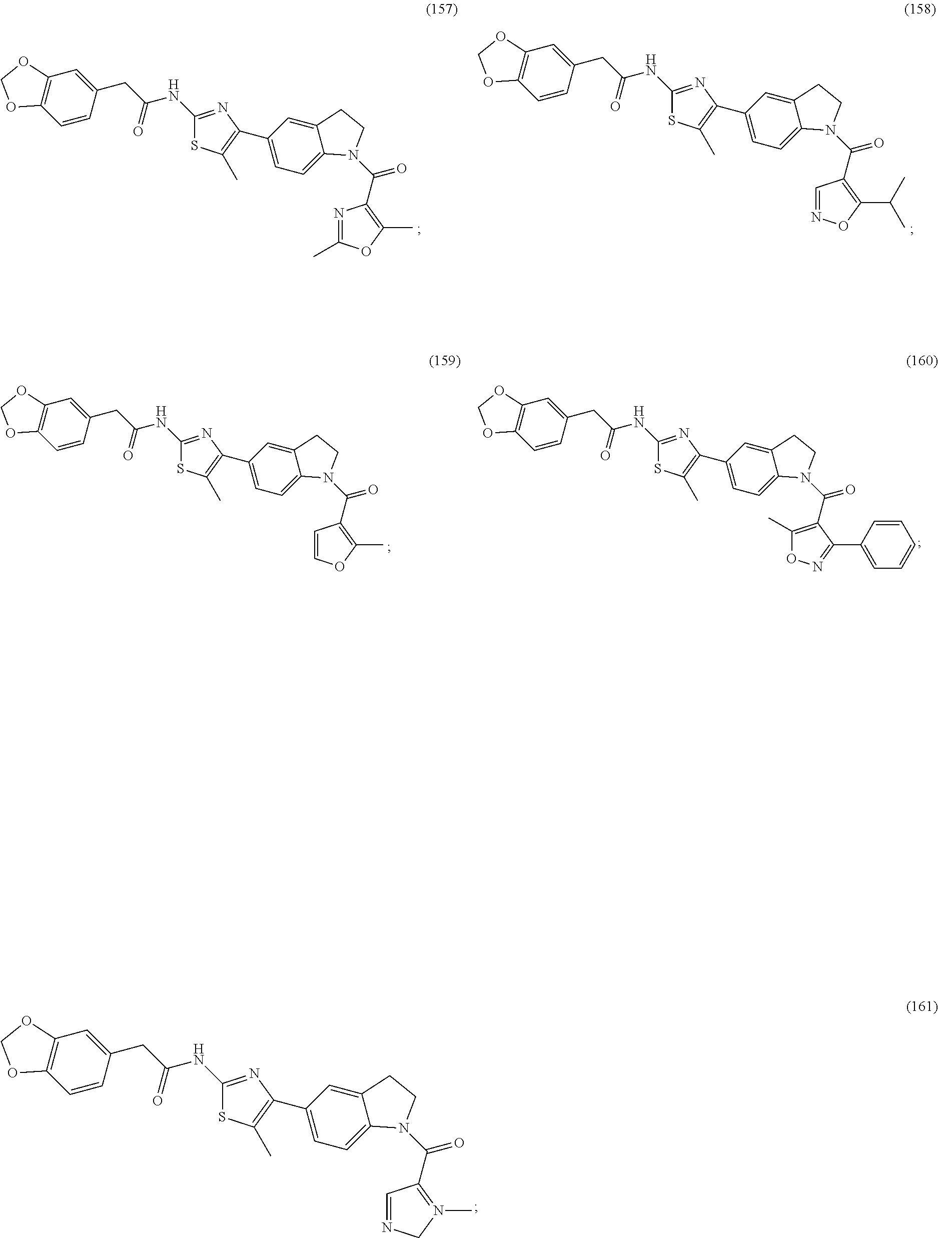

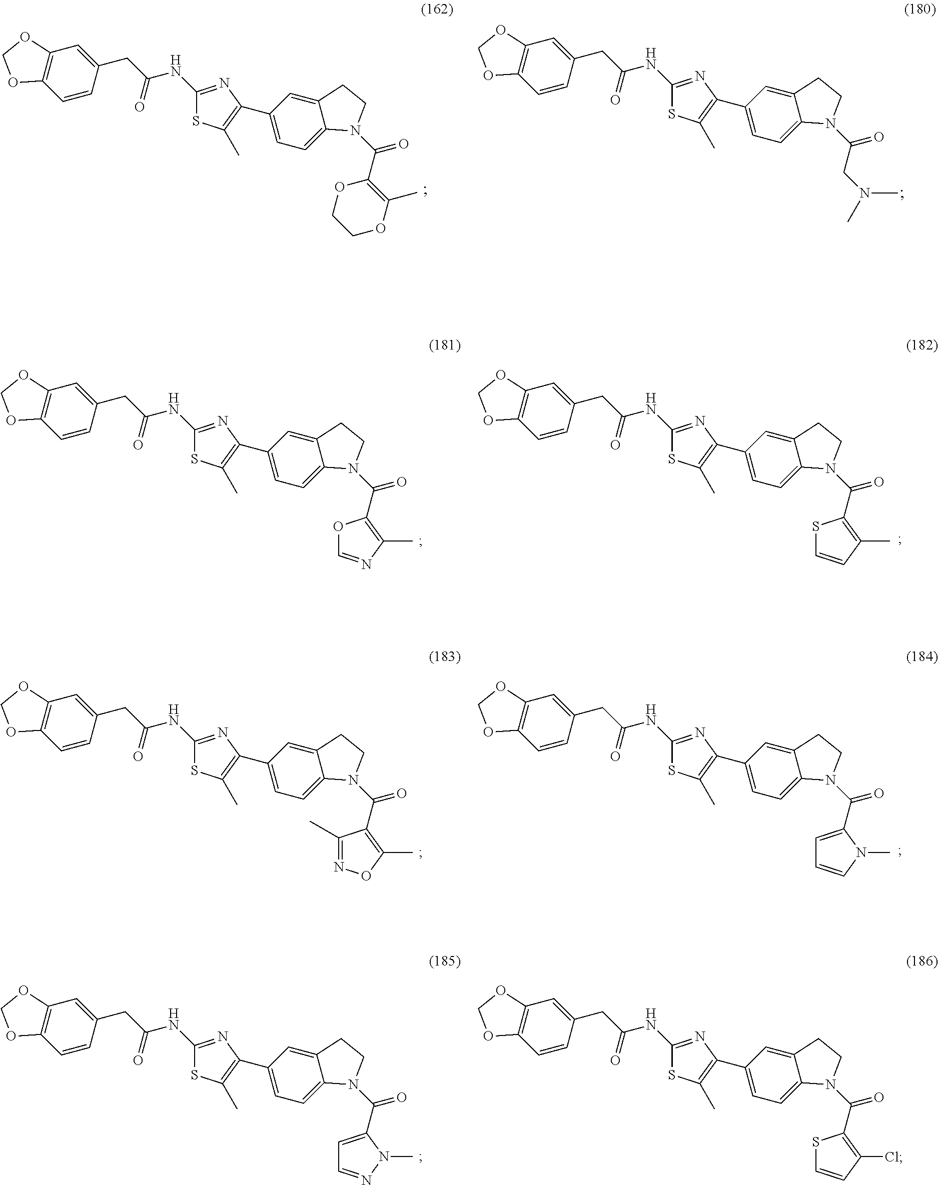

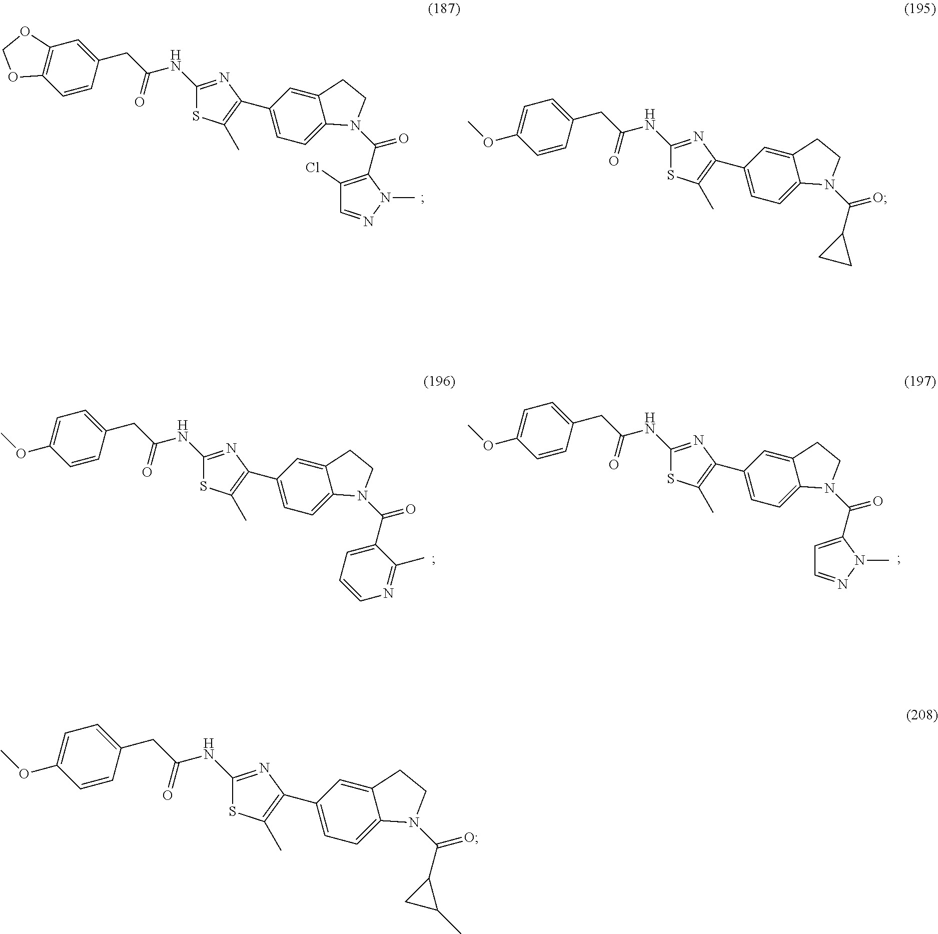

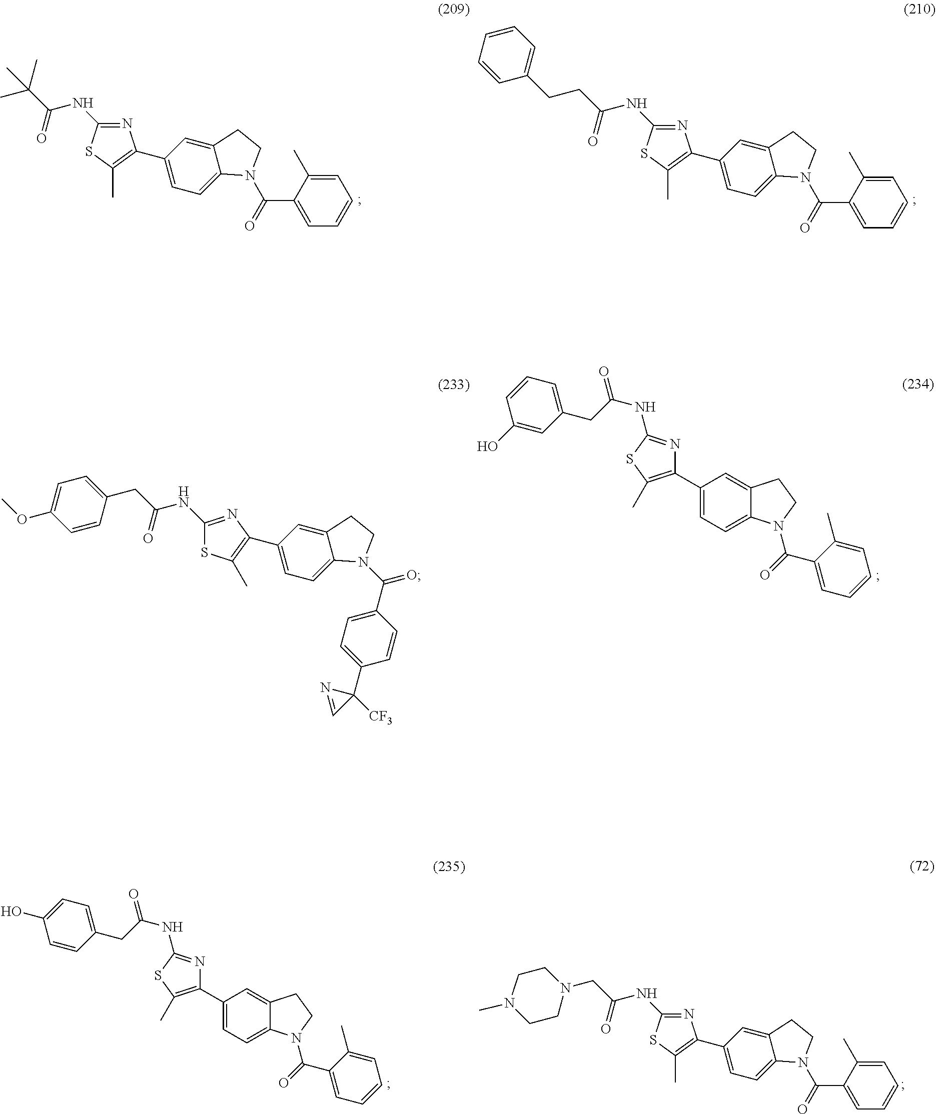 [SMILES: Cc1ccccc1C(=O)N1CCc2cc(-c3nc(NC(=O)C(C)(C)C)sc3C)ccc21 ; Cc1ccccc1C(=O)N1CCc2cc(-c3nc(NC(=O)CCc4ccccc4)sc3C)ccc21 ; COc1ccc(CC(=O)Nc2nc(-c3ccc4c(c3)CCN4C(=O)c3ccc(C4(C(F)(F)F)C=N4)cc3)c(C)s2)cc1 ; Cc1ccccc1C(=O)N1CCc2cc(-c3nc(NC(=O)Cc4cccc(O)c4)sc3C)ccc21 ; Cc1ccccc1C(=O)N1CCc2cc(-c3nc(NC(=O)Cc4ccc(O)cc4)sc3C)ccc21 ; Cc1ccccc1C(=O)N1CCc2cc(-c3nc(NC(=O)CN4CCN(C)CC4)sc3C)ccc21]

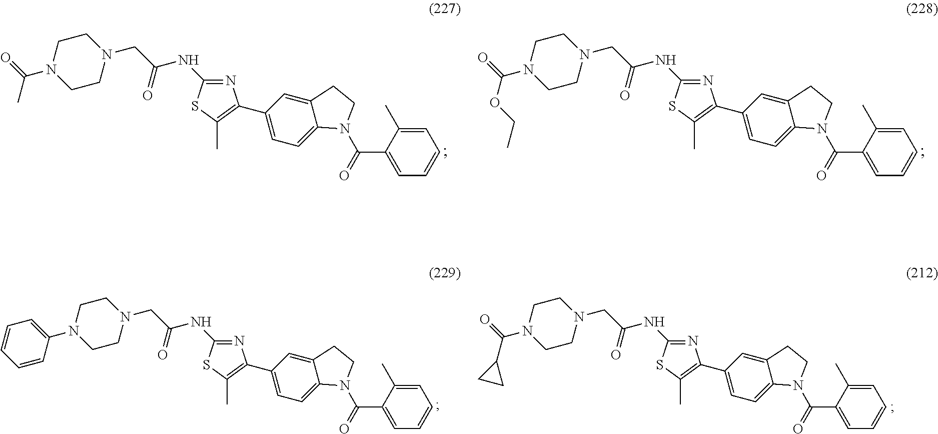 [SMILES: CC(=O)N1CCN(CC(=O)Nc2nc(-c3ccc4c(c3)CCN4C(=O)c3ccccc3C)c(C)s2)CC1 ; CCOC(=O)N1CCN(CC(=O)Nc2nc(-c3ccc4c(c3)CCN4C(=O)c3ccccc3C)c(C)s2)CC1 ; Cc1ccccc1C(=O)N1CCc2cc(-c3nc(NC(=O)CN4CCN(c5ccccc5)CC4)sc3C)ccc21 ; Cc1ccccc1C(=O)N1CCc2cc(-c3nc(NC(=O)CN4CCN(C(=O)C5CC5)CC4)sc3C)ccc21]

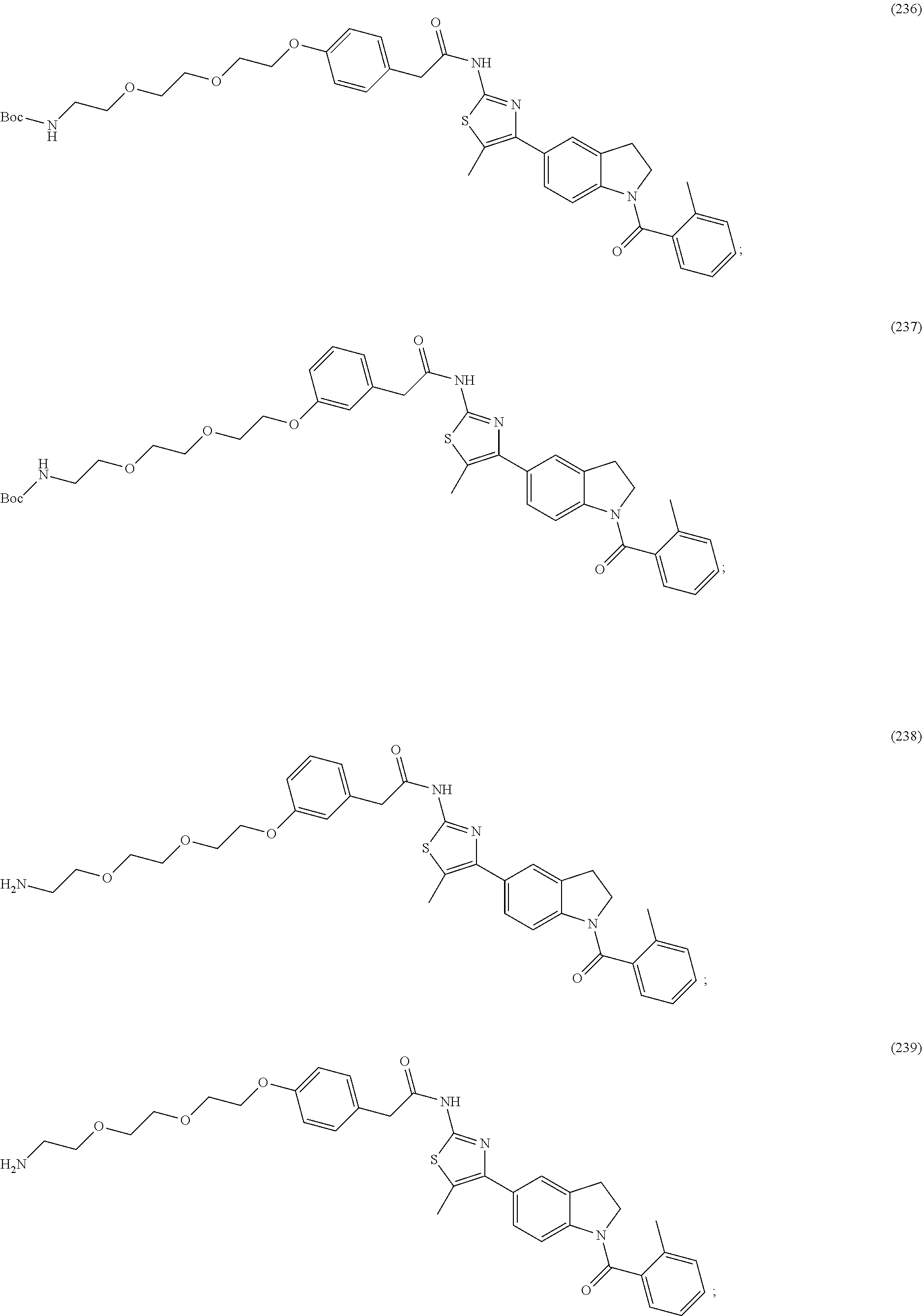 [SMILES: Cc1ccccc1C(=O)N1CCc2cc(-c3nc(NC(=O)Cc4ccc(OCCOCCOCCNC(=O)OC(C)(C)C)cc4)sc3C)ccc21 ; Cc1ccccc1C(=O)N1CCc2cc(-c3nc(NC(=O)Cc4cccc(OCCOCCOCCNC(=O)OC(C)(C)C)c4)sc3C)ccc21 ; Cc1ccccc1C(=O)N1CCc2cc(-c3nc(NC(=O)Cc4cccc(OCCOCCOCCN)c4)sc3C)ccc21 ; Cc1ccccc1C(=O)N1CCc2cc(-c3nc(NC(=O)Cc4ccc(OCCOCCOCCN)cc4)sc3C)ccc21]

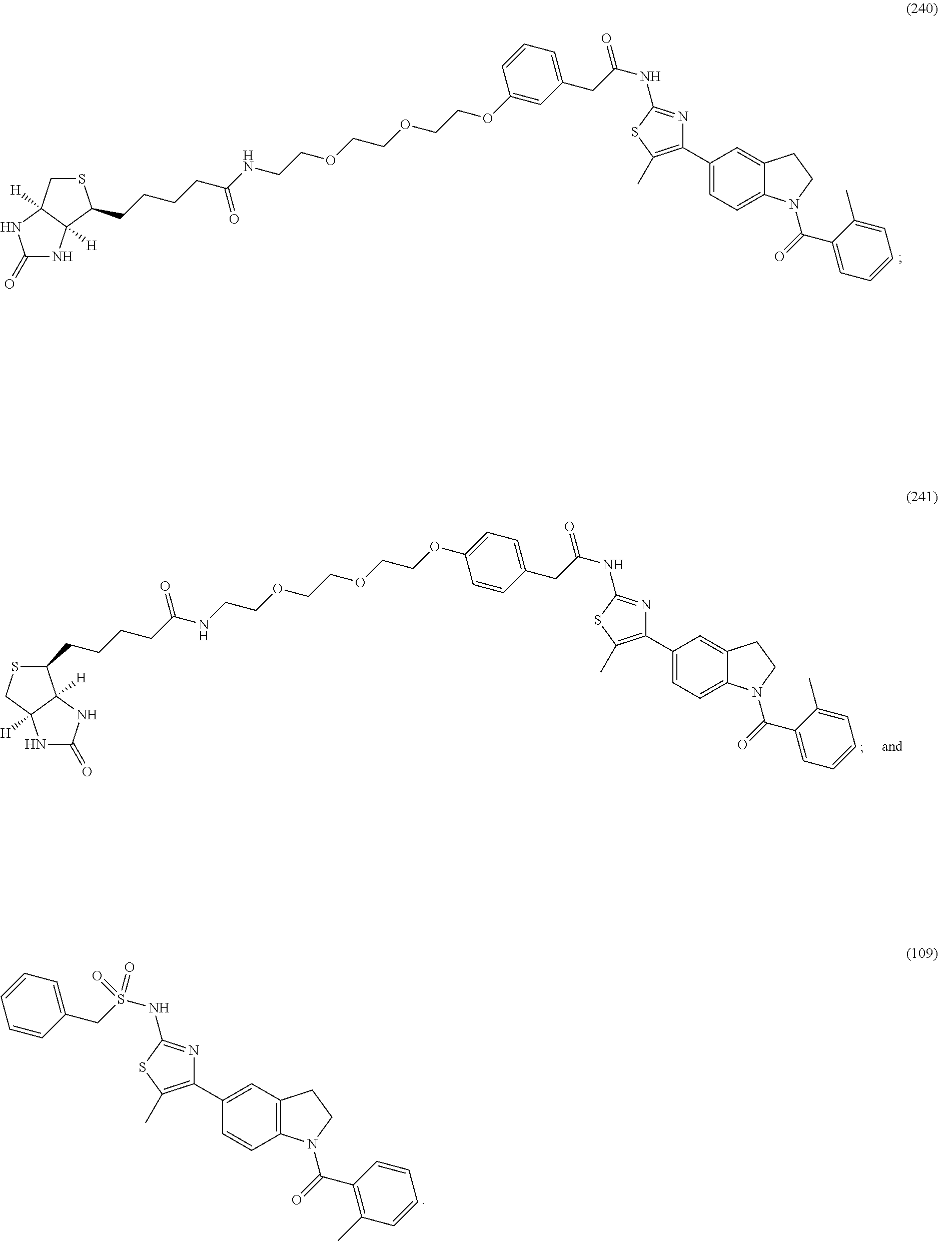 [SMILES: Cc1ccccc1C(=O)N1CCc2cc(-c3nc(NC(=O)Cc4cccc(OCCOCCOCCNC(=O)CCCC[C@@H]5SC[C@@H]6NC(=O)N[C@@H]65)c4)sc3C)ccc21 ; Cc1ccccc1C(=O)N1CCc2cc(-c3nc(NC(=O)Cc4ccc(OCCOCCOCCNC(=O)CCCC[C@@H]5SC[C@@H]6NC(=O)N[C@@H]65)cc4)sc3C)ccc21 ; Cc1ccccc1C(=O)N1CCc2cc(-c3nc(NS(=O)(=O)Cc4ccccc4)sc3C)ccc21]

4. The compound of claim 1, wherein the compound is a compound of formula (1a″):

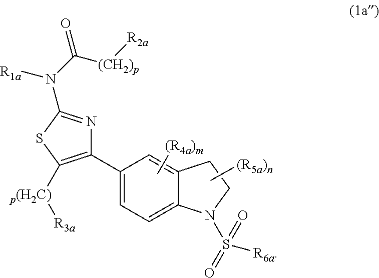

5. The compound of claim 4, wherein the compound of formula (1a″) is selected from the group consisting of:

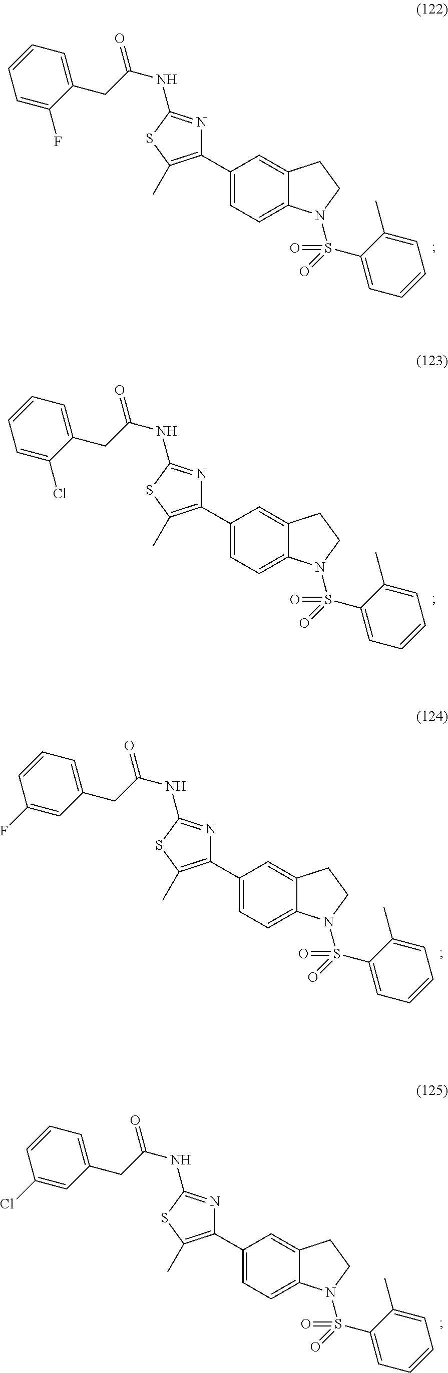

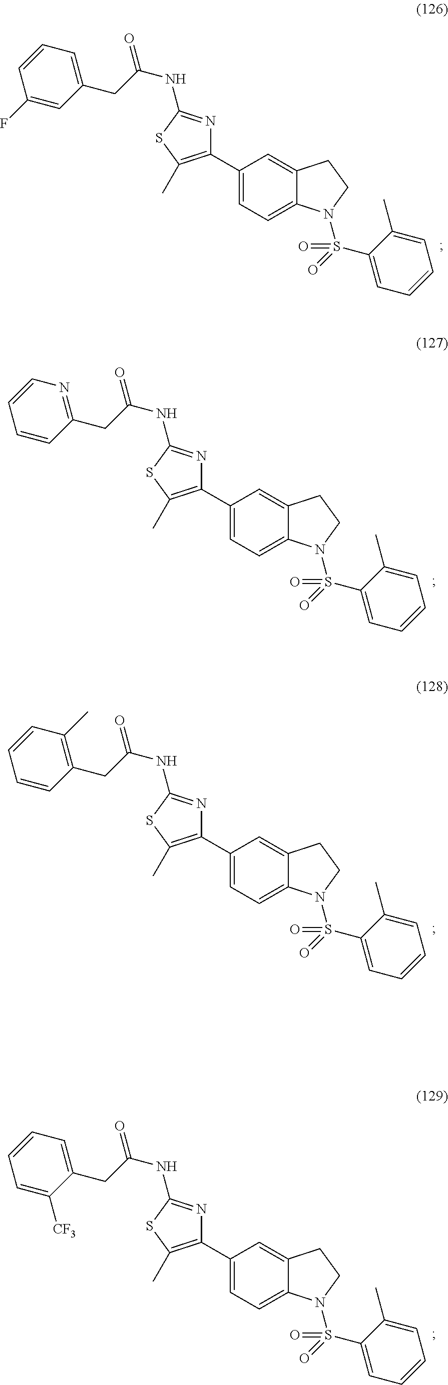

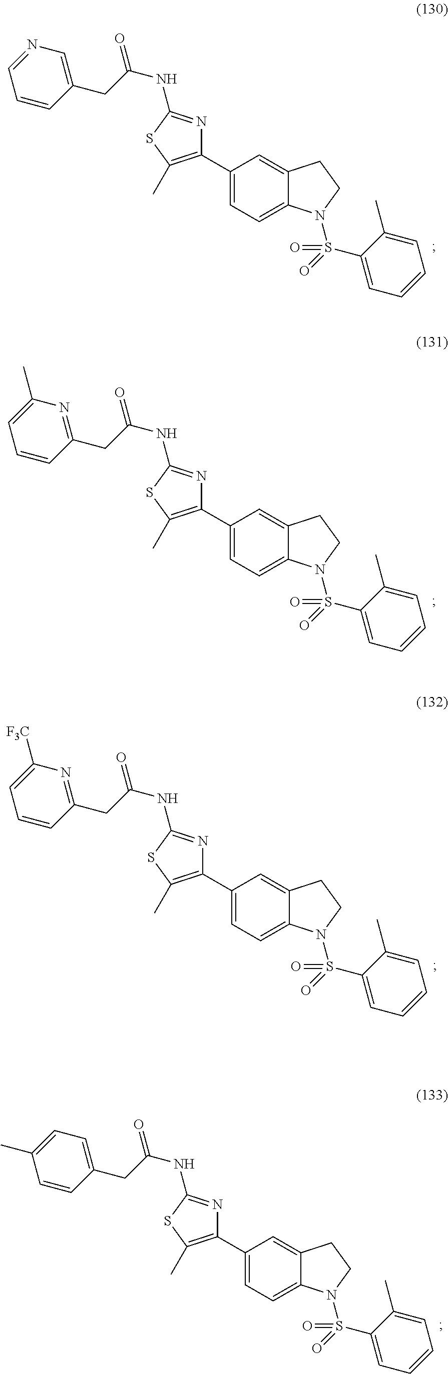

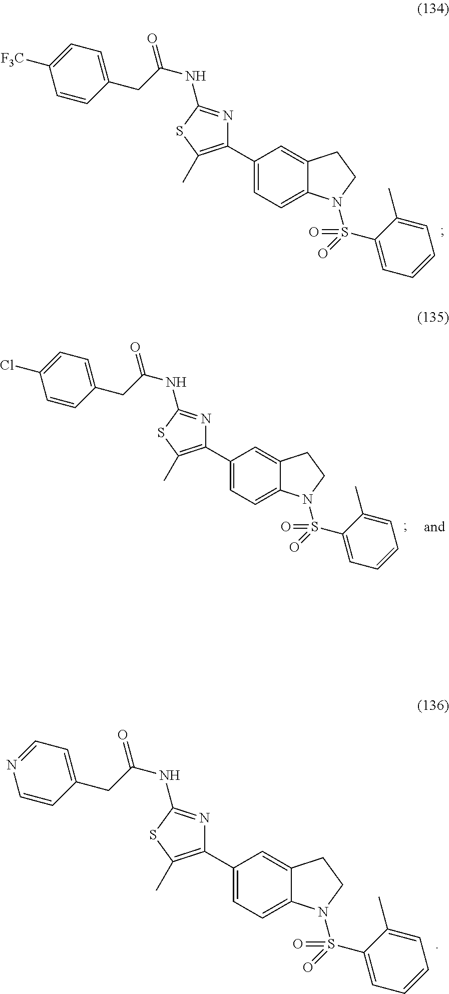

6. The compound of claim 1, wherein the compound is a compound of formula (1b′):

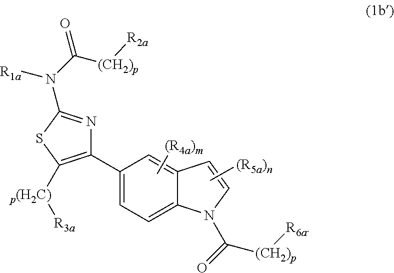

7. The compound of claim 6, wherein the compound of formula (1b′) is:

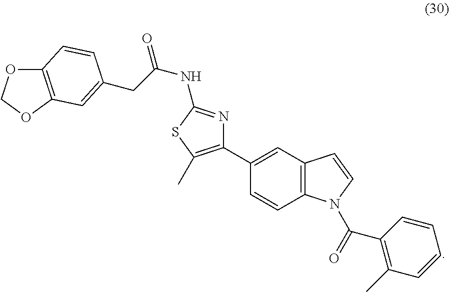 [SMILES: Cc1ccccc1C(=O)n1ccc2cc(-c3nc(NC(=O)Cc4ccc5c(c4)OCO5)sc3C)ccc21]

8. A compound selected from the group consisting of: 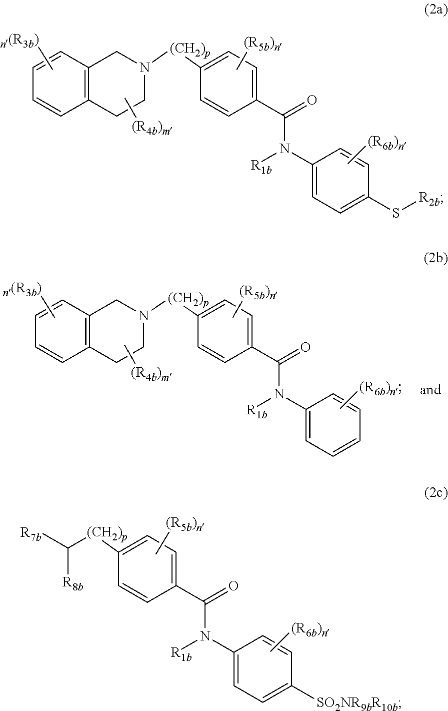
m′ is an integer selected from the group consisting of 0, 1, 2, and 3;
n′ is an integer selected from the group consisting of 0, 1, 2, 3, and 4;
each p is independently an integer selected from the group consisting of 0, 1, and 2;
R_{1b} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;
R_{2b} is selected from the group consisting of substituted or unsubstituted straight-chain or branched alkyl, substituted or unsubstituted cycloalkyl, substituted or unsubstituted cycloheteroalkyl, substituted or unsubstituted aryl, and substituted or unsubstituted heteroaryl;
each R_{3b}, R_{4b}, R_{5b}, or R_{b6} is independently selected from the group consisting of H, substituted or unsubstituted straight-chain or branched alkyl, alkenyl, alkynyl, hydroxyl, alkoxyl, halogen, amino, nitro, carbonyl, carboxyl, mercapto, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;
or for compounds of formula (2b) at least one R_{6b} is selected from the group consisting of H, substituted or unsubstituted cycloheteroalkyl, —(CH₂)ₚ—R_{6b}, and —C(═O)—R_{6b}; p is an integer selected from the group consisting of 0, 1, and 2;
R_{7b} and R_{8b} are each independently selected from the group consisting of substituted or unsubstituted straight-chain or branched alkyl and substituted or unsubstituted cycloheteroalkyl, or R_{7b} and R_{8b} can together form a substituted or unsubstituted heterocyclic ring;
R_{9b} and R_{10b} are each independently selected from the group consisting of substituted or unsubstituted straight-chain or branched alkyl and substituted or unsubstituted cycloheteroalkyl, or R_{9b} and R_{10b} can together form a substituted or unsubstituted heterocyclic ring;
or an enantiomer, diastereomer, racemate or pharmaceutically acceptable salt, prodrug, or solvate thereof;
under the proviso that if the compound is a compound of formula (2a), R_{2b} cannot be —CH₃ or —(O)₂OH.

9. The compound of claim 8, wherein the compound is a compound of formula (2b) and the compound is selected from the group consisting of:

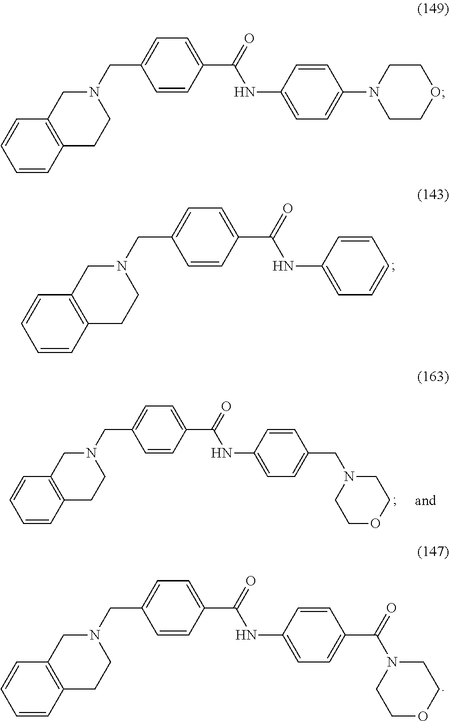 [SMILES: O=C(Nc1ccc(N2CCOCC2)cc1)c1ccc(CN2CCc3ccccc3C2)cc1 ; O=C(Nc1ccccc1)c1ccc(CN2CCc3ccccc3C2)cc1 ; O=C(Nc1ccc(CN2CCOCC2)cc1)c1ccc(CN2CCc3ccccc3C2)cc1 ; O=C(Nc1ccc(C(=O)N2CCOCC2)cc1)c1ccc(CN2CCc3ccccc3C2)cc1]

10. The compound of claim 8, wherein the compound is a compound of formula (2c) and the compound is selected from the group consisting of:

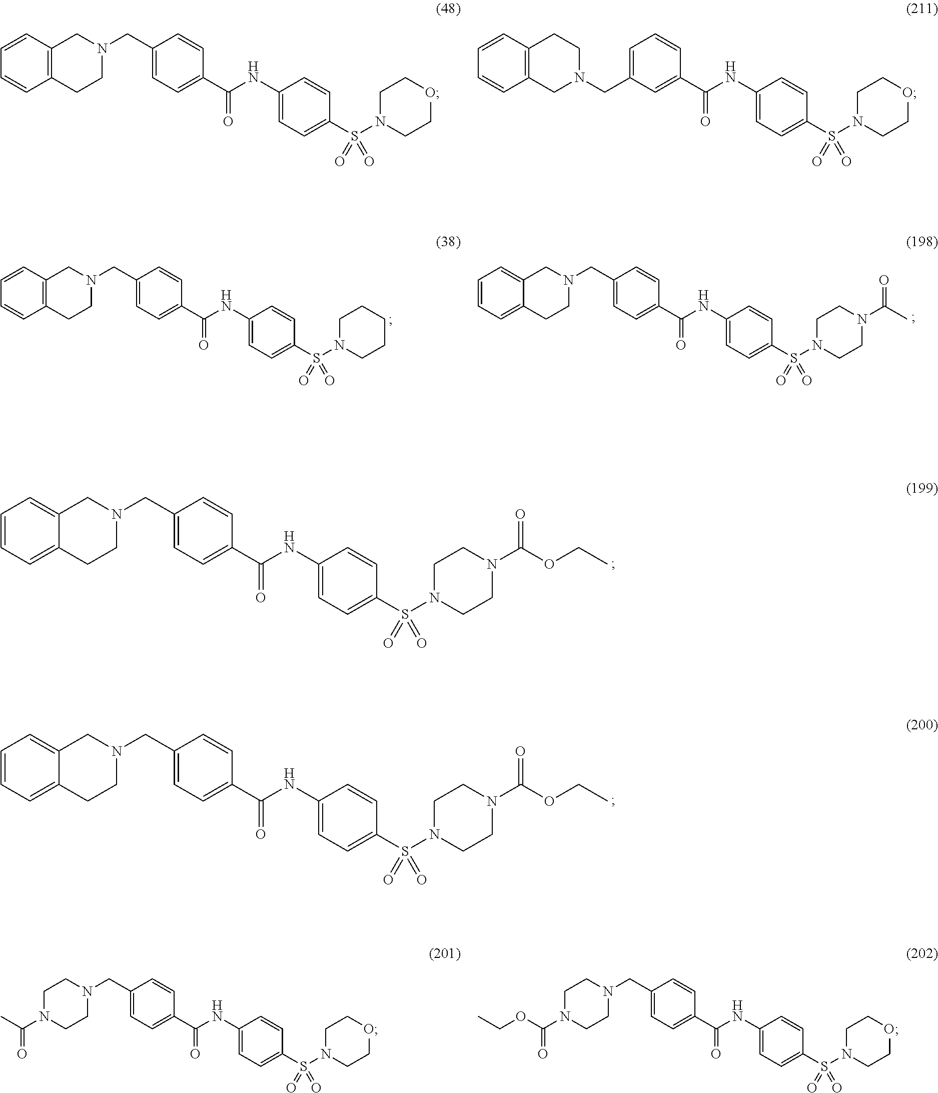

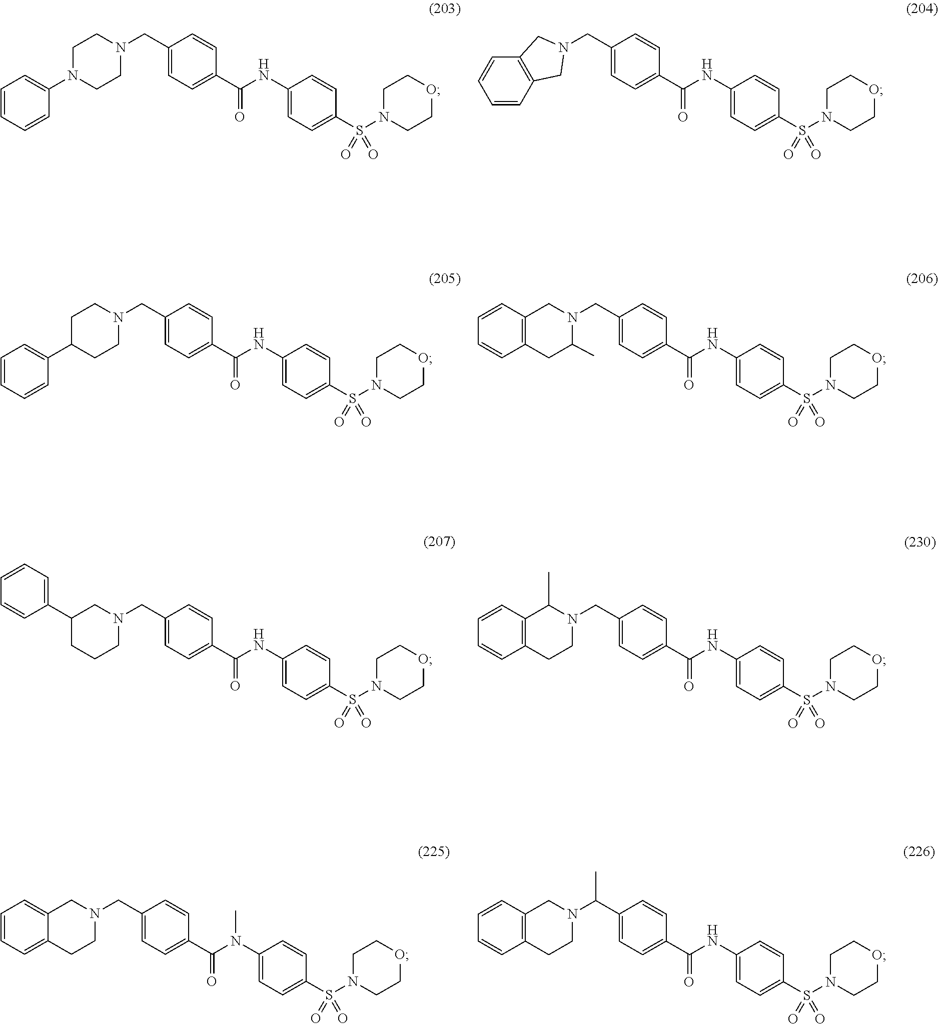

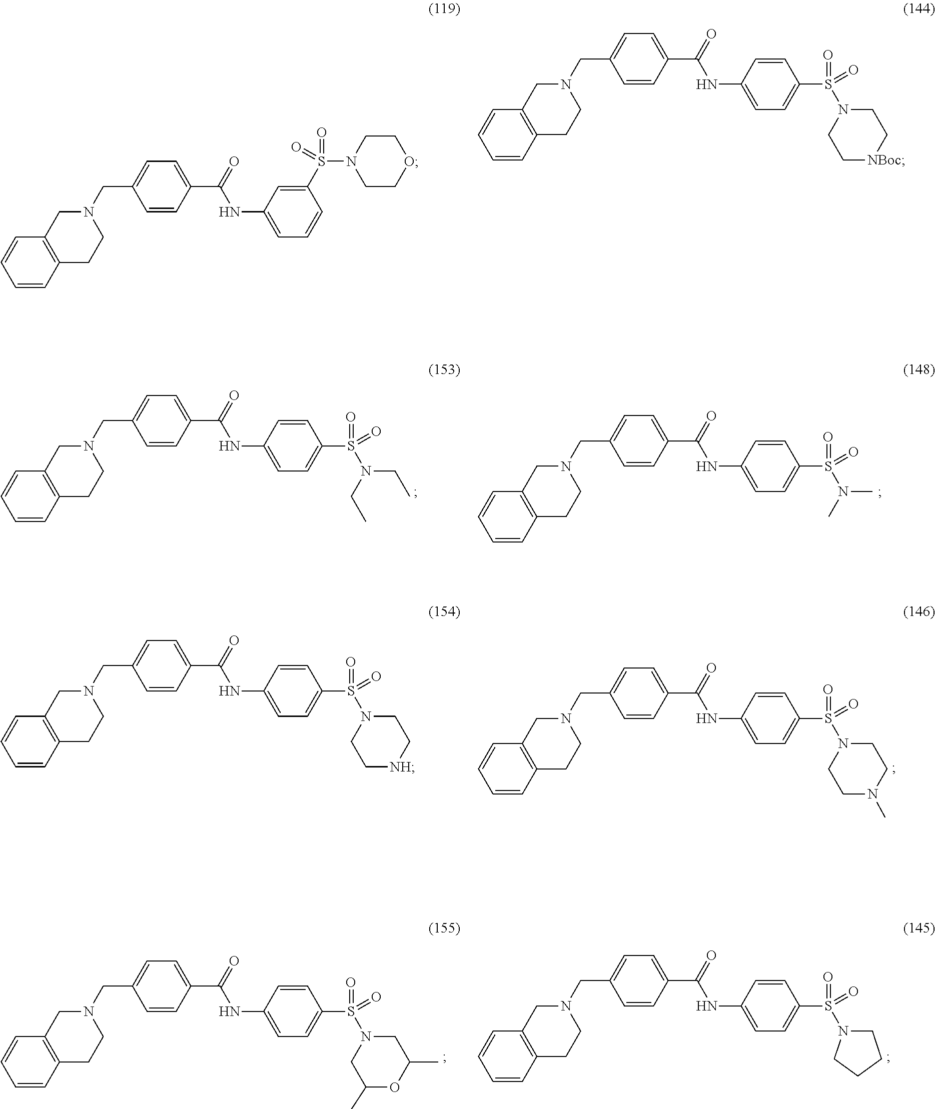

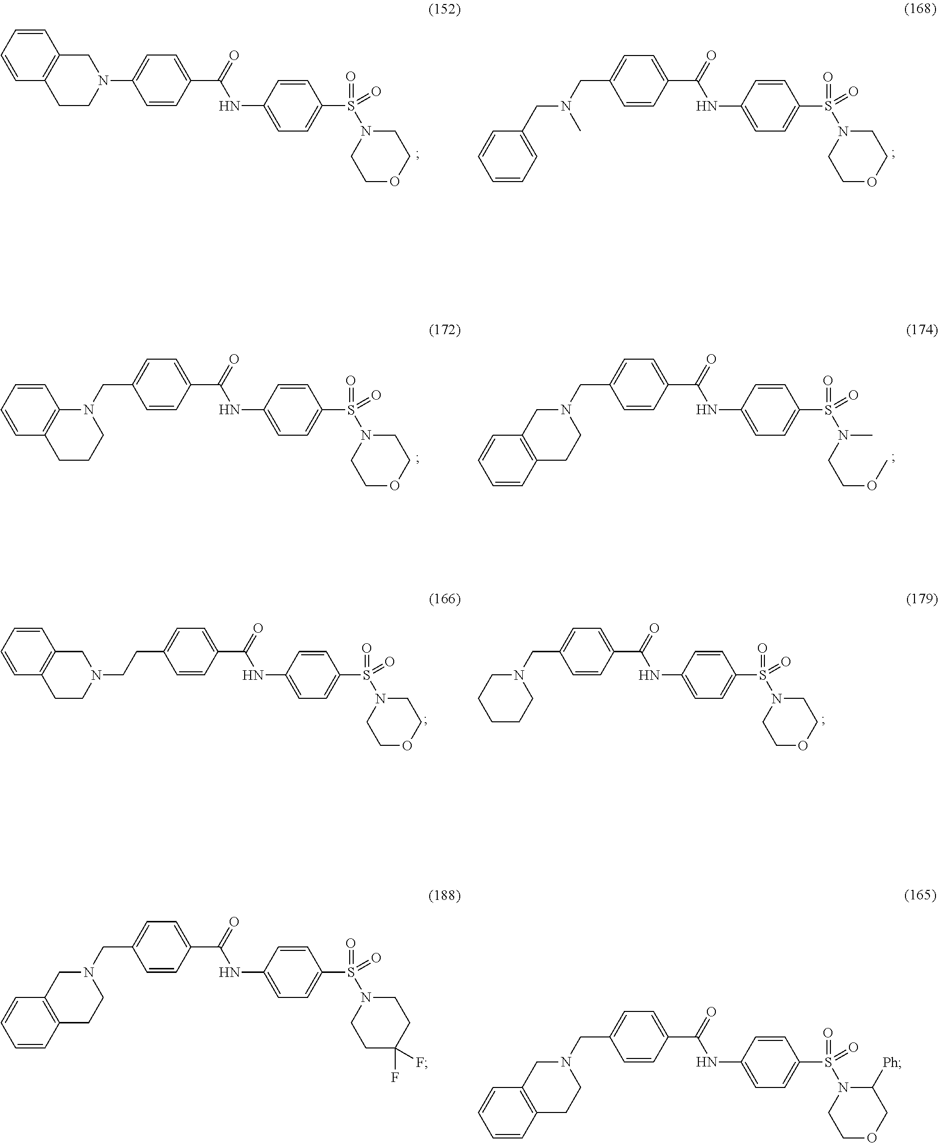

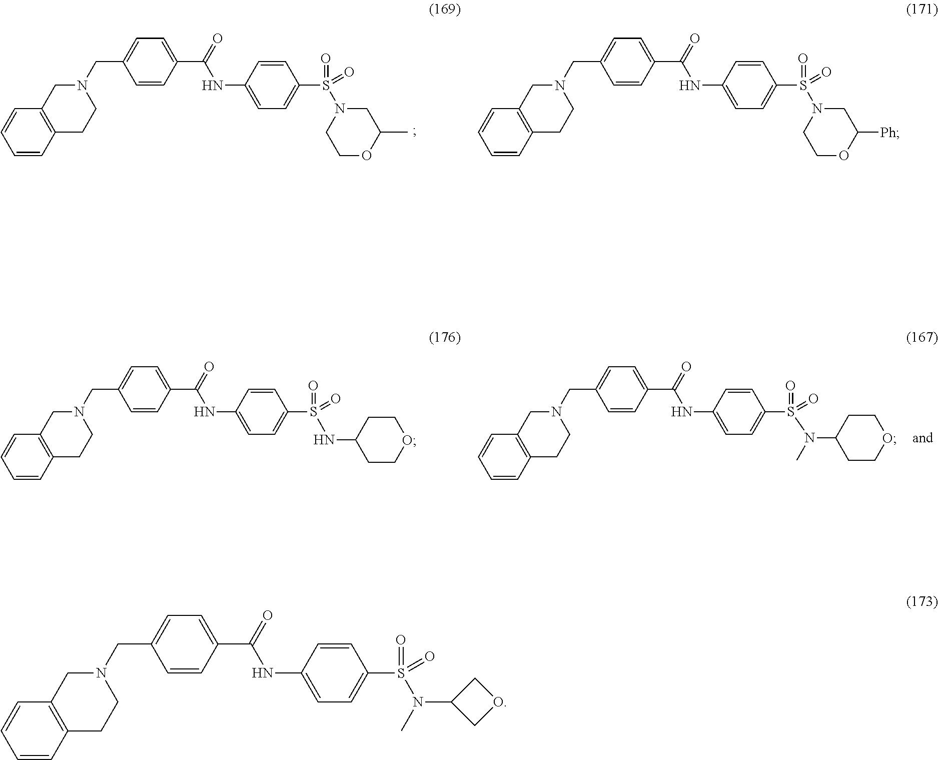 [SMILES: CC1CN(S(=O)(=O)c2ccc(NC(=O)c3ccc(CN4CCc5ccccc5C4)cc3)cc2)CCO1 ; O=C(Nc1ccc(S(=O)(=O)N2CCOC(c3ccccc3)C2)cc1)c1ccc(CN2CCc3ccccc3C2)cc1 ; O=C(Nc1ccc(S(=O)(=O)NC2CCOCC2)cc1)c1ccc(CN2CCc3ccccc3C2)cc1 ; CN(C1CCOCC1)S(=O)(=O)c1ccc(NC(=O)c2ccc(CN3CCc4ccccc4C3)cc2)cc1 ; CN(C1COC1)S(=O)(=O)c1ccc(NC(=O)c2ccc(CN3CCc4ccccc4C3)cc2)cc1]

11. A compound of formula (3): 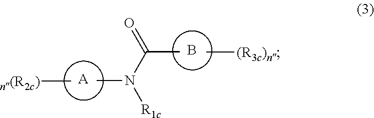
wherein:
each n″ is an integer independently selected from the group consisting of 0, 1, 2, 3, 4, 5, and 6, depending on the maximum available atoms on ring A and ring B;
A is a ring structure selected from the group consisting of: 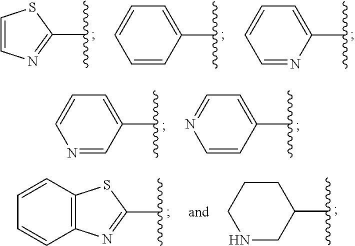
B is —(CH₂)ₙ— or a ring structure selected from the group consisting of: 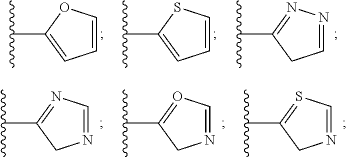
wherein the ring structure A and ring structure B are connected via an amide linkage represented by —NR_{1c}C(═O)—;
R_{1c} is selected from the group consisting of H, substituted or unsubstituted straight-chain or branched alkyl, hydroxyl, alkoxyl, substituted or unsubstituted heteroalkyl, substituted or unsubstituted cycloalkyl, substituted or unsubstituted cycloheteroalkyl, substituted or unsubstituted aryl, and substituted or unsubstituted heteroaryl;
R_{2c} and R_{3c} are each independently selected from the group consisting of substituted or unsubstituted straight-chain or branched alkyl and —(CH₂)ₚ-Cy,
wherein p is an integer selected from the group consisting of 0, 1, and 2; and Cy is selected from the group consisting of substituted or unsubstituted cycloheteroalkyl, substituted or unsubstituted aryl, and substituted or unsubstituted heteroaryl;
or an enantiomer, diastereomer, racemate or pharmaceutically acceptable salt, prodrug, or solvate thereof.

12. The compound of claim 11, wherein the compound of formula (3) is selected from the group consisting of:

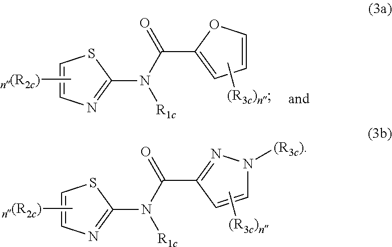

13. The compound of claim 11, wherein the compound is a compound of Formula (3a) and the compound is selected from the group consisting of:

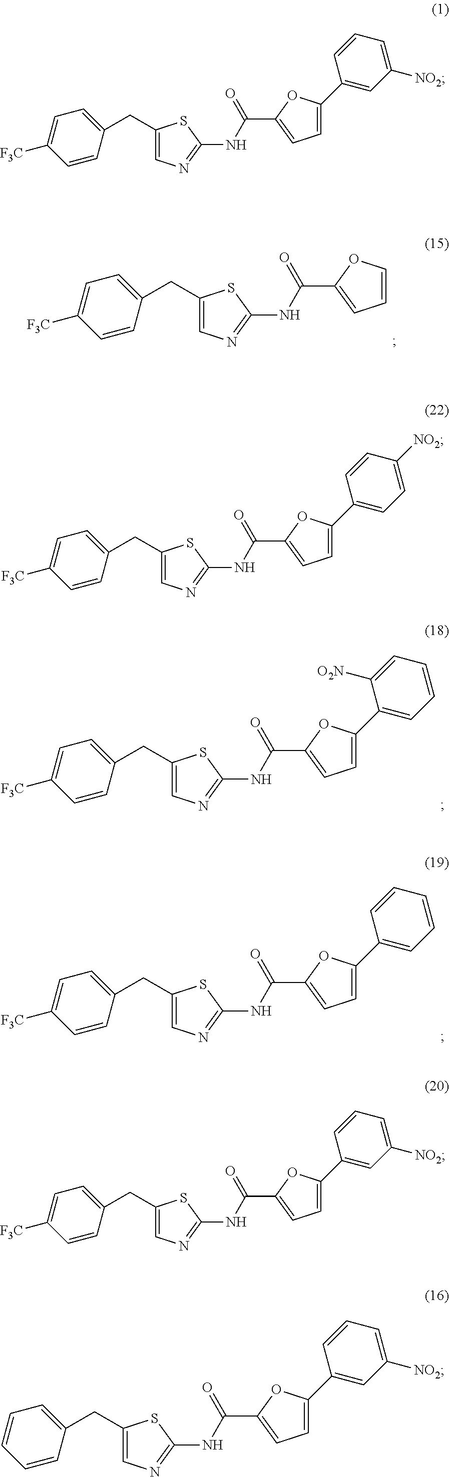

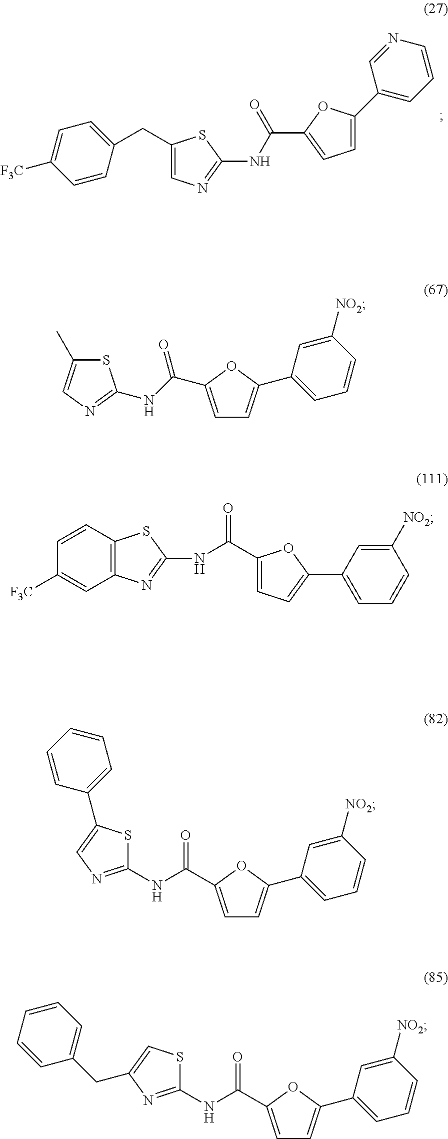 [SMILES: O=C(Nc1ncc(Cc2ccc(C(F)(F)F)cc2)s1)c1ccc(-c2cccnc2)o1 ; ";" ; Cc1cnc(NC(=O)c2ccc(-c3cccc([N+](=O)[O-])c3)o2)s1 ; O=C(Nc1nc2cc(C(F)(F)F)ccc2s1)c1ccc(-c2cccc([N+](=O)[O-])c2)o1 ; O=C(Nc1ncc(-c2ccccc2)s1)c1ccc(-c2cccc([N+](=O)[O-])c2)o1 ; O=C(Nc1nc(Cc2ccccc2)cs1)c1ccc(-c2cccc([N+](=O)[O-])c2)o1]

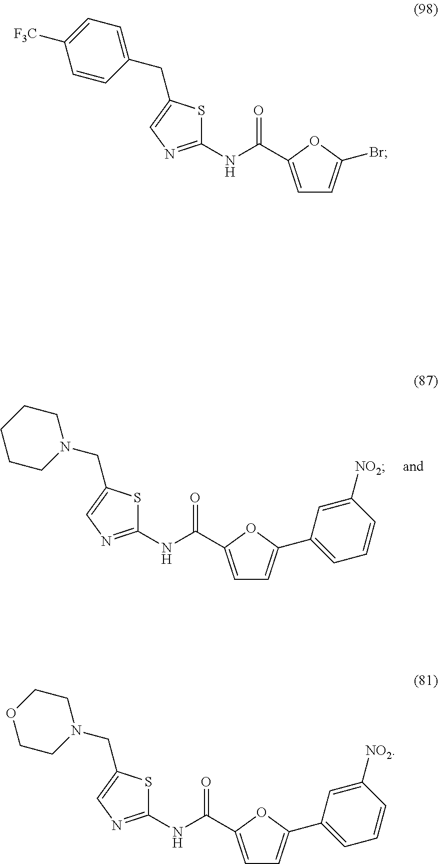 [SMILES: O=C(Nc1ncc(Cc2ccc(C(F)(F)F)cc2)s1)c1ccc(Br)o1 ; O=C(Nc1ncc(CN2CCCCC2)s1)c1ccc(-c2cccc([N+](=O)[O-])c2)o1 ; "and" ; O=C(Nc1ncc(CN2CCOCC2)s1)c1ccc(-c2cccc([N+](=O)[O-])c2)o1]

14. The compound of claim 11, wherein the compound is a compound of Formula (3b) and the compound is selected from the group consisting of:

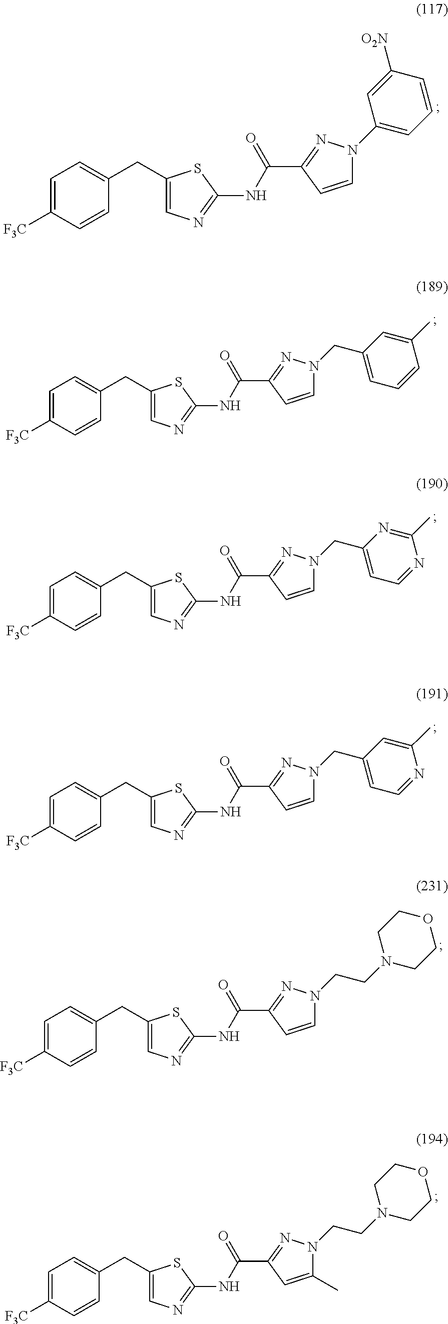 [SMILES: O=C(Nc1ncc(Cc2ccc(C(F)(F)F)cc2)s1)c1ccn(-c2cccc([N+](=O)[O-])c2)n1 ; Cc1cccc(Cn2ccc(C(=O)Nc3ncc(Cc4ccc(C(F)(F)F)cc4)s3)n2)c1 ; Cc1nccc(Cn2ccc(C(=O)Nc3ncc(Cc4ccc(C(F)(F)F)cc4)s3)n2)n1 ; Cc1cc(Cn2ccc(C(=O)Nc3ncc(Cc4ccc(C(F)(F)F)cc4)s3)n2)ccn1 ; O=C(Nc1ncc(Cc2ccc(C(F)(F)F)cc2)s1)c1ccn(CCN2CCOCC2)n1 ; Cc1cc(C(=O)Nc2ncc(Cc3ccc(C(F)(F)F)cc3)s2)nn1CCN1CCOCC1]

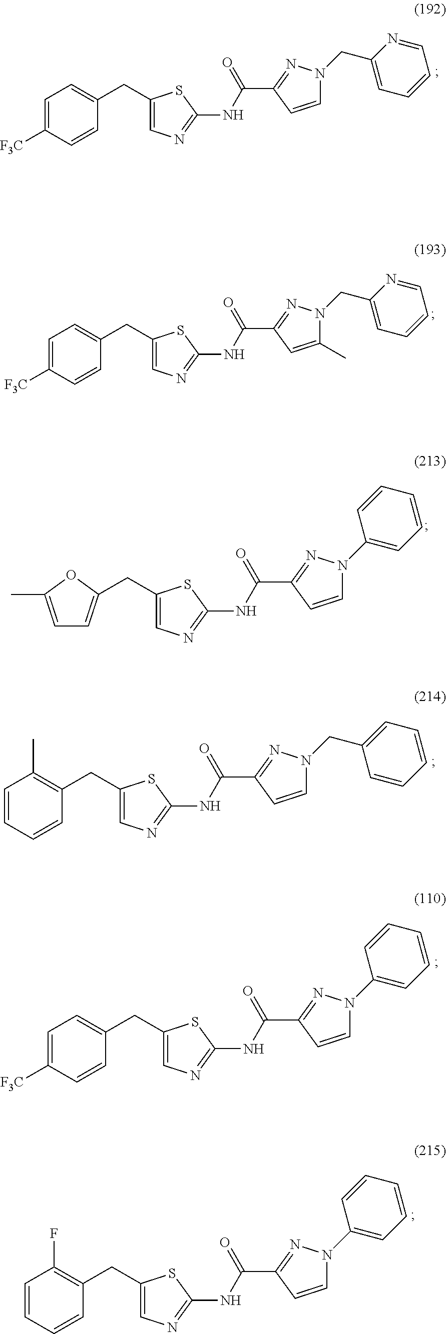 [SMILES: O=C(Nc1ncc(Cc2ccc(C(F)(F)F)cc2)s1)c1ccn(Cc2ccccn2)n1 ; Cc1cc(C(=O)Nc2ncc(Cc3ccc(C(F)(F)F)cc3)s2)nn1Cc1ccccn1 ; Cc1ccc(Cc2cnc(NC(=O)c3ccn(-c4ccccc4)n3)s2)o1 ; Cc1ccccc1Cc1cnc(NC(=O)c2ccn(Cc3ccccc3)n2)s1 ; O=C(Nc1ncc(Cc2ccc(C(F)(F)F)cc2)s1)c1ccn(-c2ccccc2)n1 ; O=C(Nc1ncc(Cc2ccccc2F)s1)c1ccn(-c2ccccc2)n1]

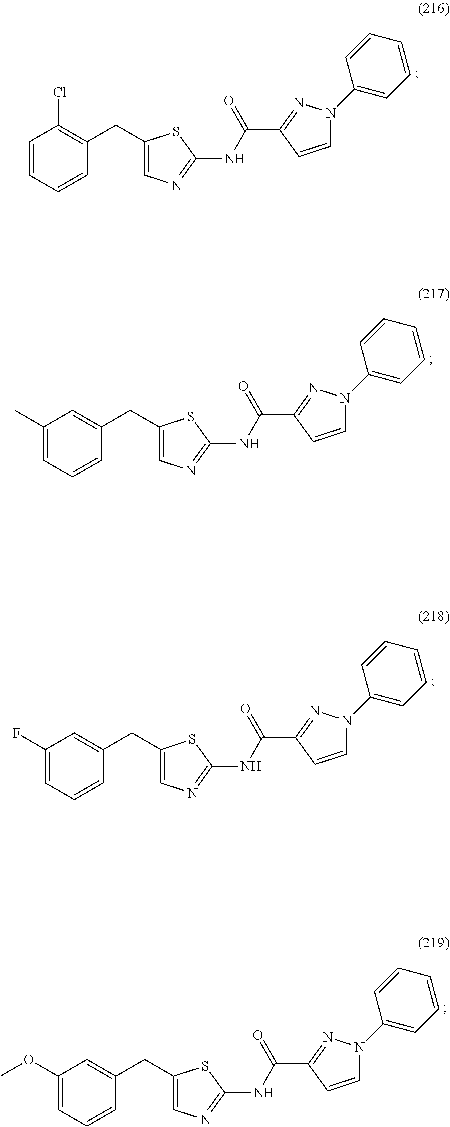 [SMILES: O=C(Nc1ncc(Cc2ccccc2Cl)s1)c1ccn(-c2ccccc2)n1 ; Cc1cccc(Cc2cnc(NC(=O)c3ccn(-c4ccccc4)n3)s2)c1 ; O=C(Nc1ncc(Cc2cccc(F)c2)s1)c1ccn(-c2ccccc2)n1 ; COc1cccc(Cc2cnc(NC(=O)c3ccn(-c4ccccc4)n3)s2)c1]

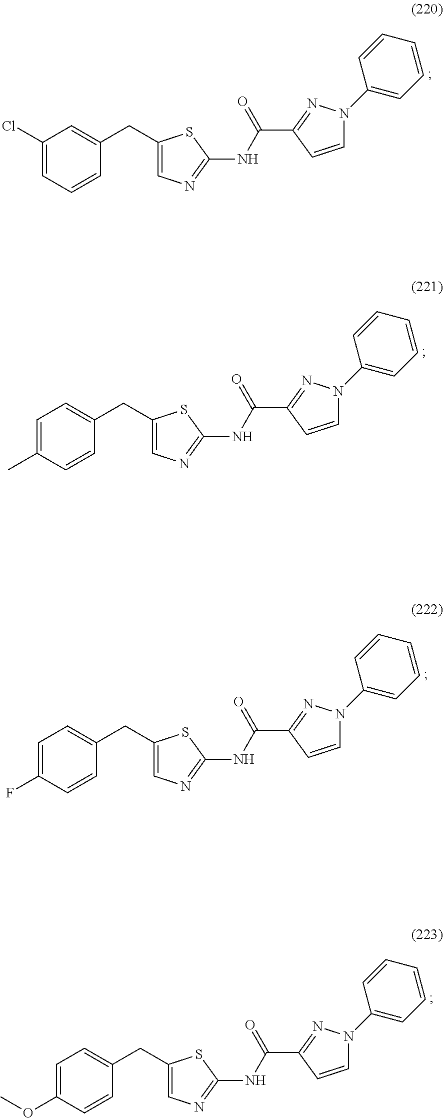 [SMILES: O=C(Nc1ncc(Cc2cccc(Cl)c2)s1)c1ccn(-c2ccccc2)n1 ; Cc1ccc(Cc2cnc(NC(=O)c3ccn(-c4ccccc4)n3)s2)cc1 ; O=C(Nc1ncc(Cc2ccc(F)cc2)s1)c1ccn(-c2ccccc2)n1 ; COc1ccc(Cc2cnc(NC(=O)c3ccn(-c4ccccc4)n3)s2)cc1]

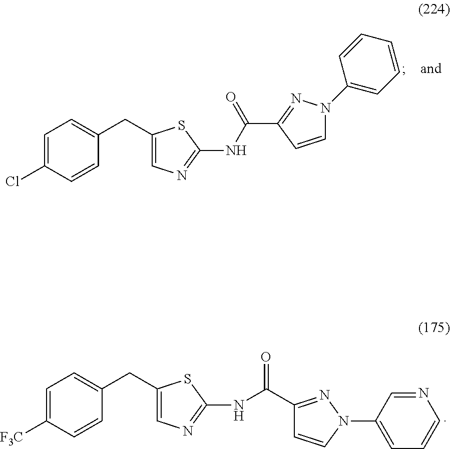 [SMILES: O=C(Nc1ncc(Cc2ccc(Cl)cc2)s1)c1ccn(-c2ccccc2)n1 ; O=C(Nc1ncc(Cc2ccc(C(F)(F)F)cc2)s1)c1ccn(-c2cccnc2)n1]

15. The compound of claim 11, wherein:
A is thiazolyl;
B is selected from the group consisting of phenyl, pyridinyl, imidazolyl, oxazolyl, thiophenyl, thiazolyl, and —(CH₂)ₙ—; and
the compound is selected from the group consisting of:

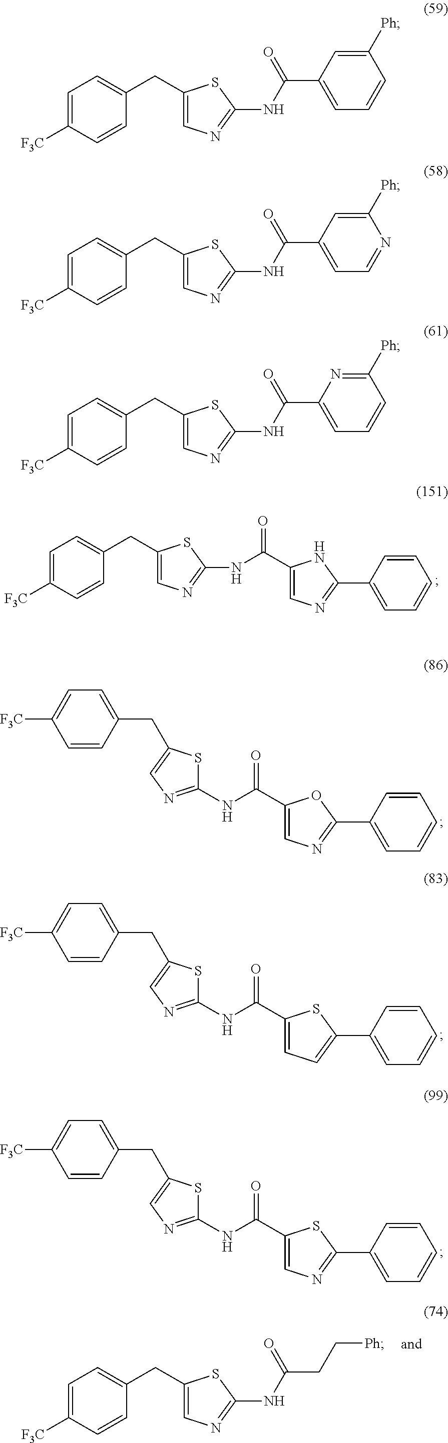

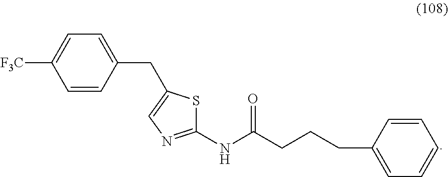 [SMILES: O=C(CCCc1ccccc1)Nc1ncc(Cc2ccc(C(F)(F)F)cc2)s1]

16. The compound of claim 11, wherein:
A is selected from the group consisting of phenyl, pyridinyl, and piperidinyl;
B is furanyl; and
the compound is selected from the group consisting of:

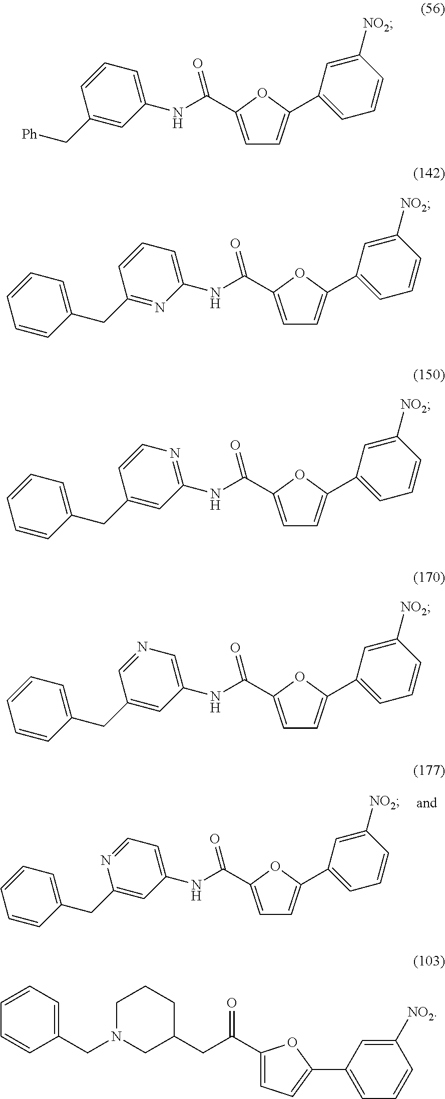 [SMILES: O=C(Nc1cccc(Cc2ccccc2)c1)c1ccc(-c2cccc([N+](=O)[O-])c2)o1 ; O=C(Nc1cccc(Cc2ccccc2)n1)c1ccc(-c2cccc([N+](=O)[O-])c2)o1 ; O=C(Nc1cc(Cc2ccccc2)ccn1)c1ccc(-c2cccc([N+](=O)[O-])c2)o1 ; O=C(Nc1cncc(Cc2ccccc2)c1)c1ccc(-c2cccc([N+](=O)[O-])c2)o1 ; O=C(Nc1ccnc(Cc2ccccc2)c1)c1ccc(-c2cccc([N+](=O)[O-])c2)o1 ; O=C(CC1CCCN(Cc2ccccc2)C1)c1ccc(-c2cccc([N+](=O)[O-])c2)o1]

17. The compound of claim 11, wherein:
A is phenyl;
B is selected from the group consisting of pyridinyl, pyrimidinyl, pyrrolidinyl, piperidinyl, and pyrazolyl; and
the compound is selected from the group consisting of:

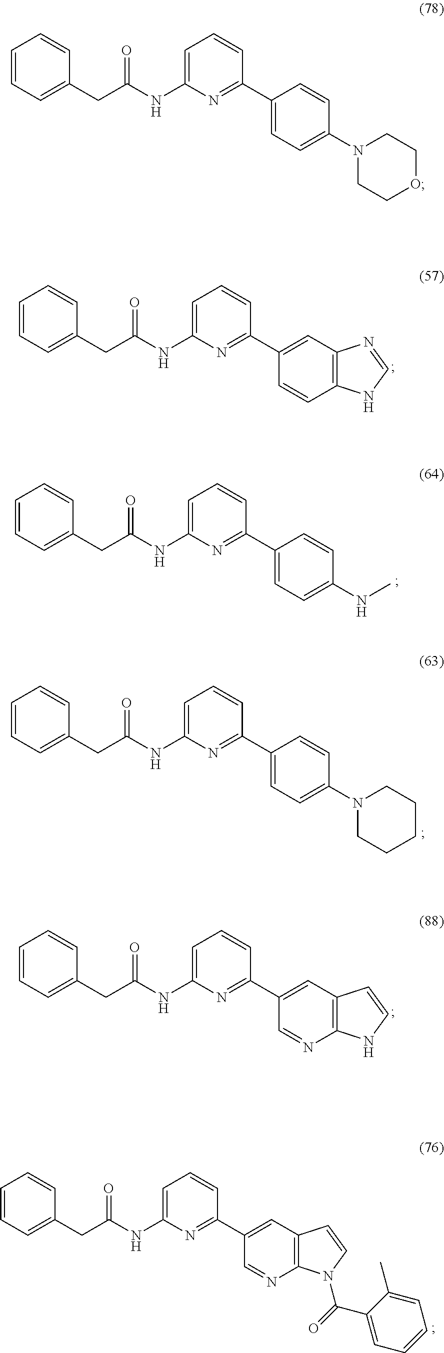 [SMILES: O=C(Cc1ccccc1)Nc1cccc(-c2ccc(N3CCOCC3)cc2)n1 ; O=C(Cc1ccccc1)Nc1cccc(-c2ccc3[nH]cnc3c2)n1 ; CNc1ccc(-c2cccc(NC(=O)Cc3ccccc3)n2)cc1 ; O=C(Cc1ccccc1)Nc1cccc(-c2ccc(N3CCCCC3)cc2)n1 ; O=C(Cc1ccccc1)Nc1cccc(-c2cnc3[nH]ccc3c2)n1 ; Cc1ccccc1C(=O)n1ccc2cc(-c3cccc(NC(=O)Cc4ccccc4)n3)cnc21]

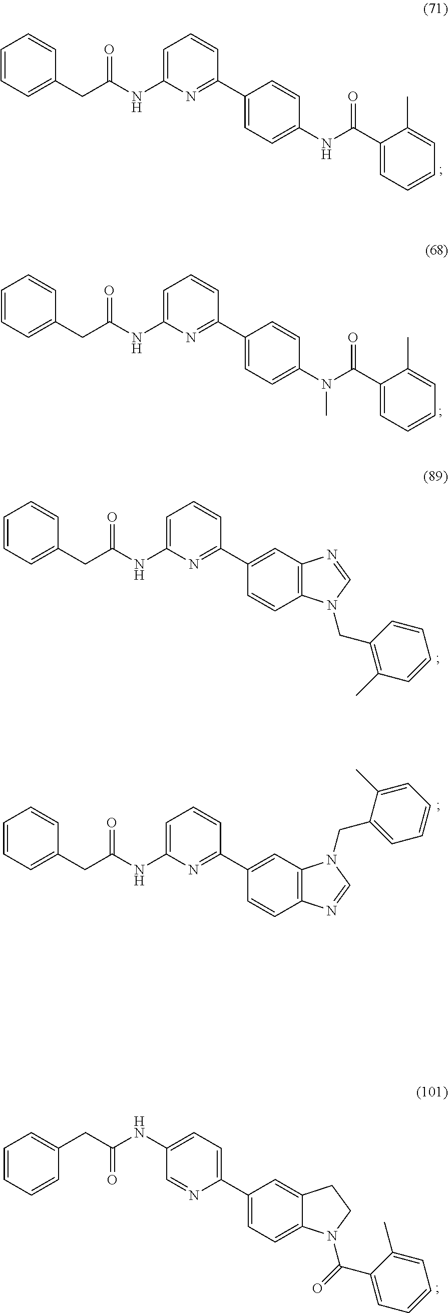 [SMILES: Cc1ccccc1C(=O)Nc1ccc(-c2cccc(NC(=O)Cc3ccccc3)n2)cc1 ; Cc1ccccc1C(=O)N(C)c1ccc(-c2cccc(NC(=O)Cc3ccccc3)n2)cc1 ; Cc1ccccc1Cn1cnc2cc(-c3cccc(NC(=O)Cc4ccccc4)n3)ccc21 ; Cc1ccccc1Cn1cnc2ccc(-c3cccc(NC(=O)Cc4ccccc4)n3)cc21 ; Cc1ccccc1C(=O)N1CCc2cc(-c3ccc(NC(=O)Cc4ccccc4)cn3)ccc21]

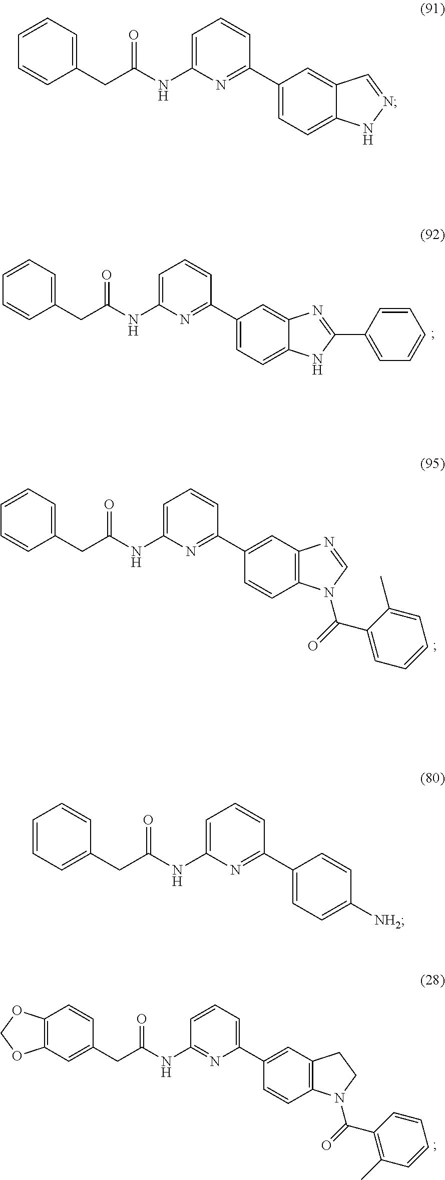 [SMILES: O=C(Cc1ccccc1)Nc1cccc(-c2ccc3[nH]ncc3c2)n1 ; O=C(Cc1ccccc1)Nc1cccc(-c2ccc3[nH]c(-c4ccccc4)nc3c2)n1 ; Cc1ccccc1C(=O)n1cnc2cc(-c3cccc(NC(=O)Cc4ccccc4)n3)ccc21 ; Nc1ccc(-c2cccc(NC(=O)Cc3ccccc3)n2)cc1 ; Cc1ccccc1C(=O)N1CCc2cc(-c3cccc(NC(=O)Cc4ccc5c(c4)OCO5)n3)ccc21]

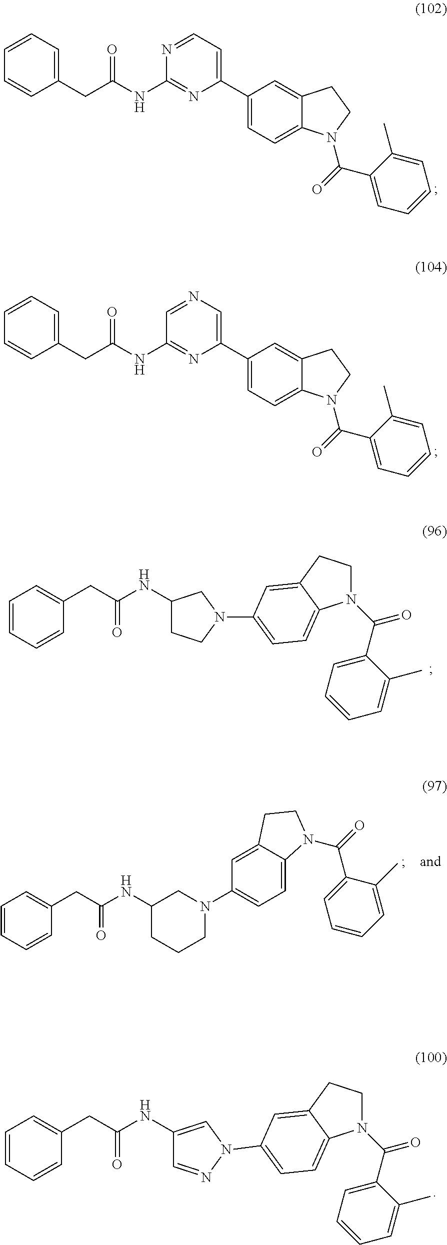 [SMILES: Cc1ccccc1C(=O)N1CCc2cc(-c3ccnc(NC(=O)Cc4ccccc4)n3)ccc21 ; Cc1ccccc1C(=O)N1CCc2cc(-c3cncc(NC(=O)Cc4ccccc4)n3)ccc21 ; Cc1ccccc1C(=O)N1CCc2cc(N3CCC(NC(=O)Cc4ccccc4)C3)ccc21 ; Cc1ccccc1C(=O)N1CCc2cc(N3CCCC(NC(=O)Cc4ccccc4)C3)ccc21 ; Cc1ccccc1C(=O)N1CCc2cc(-n3cc(NC(=O)Cc4ccccc4)cn3)ccc21]

18. The compound of claim 11, wherein:
A is phenyl;
B forms an indolinyl ring structure with the amide linkage; and
the compound is selected from the group consisting of:

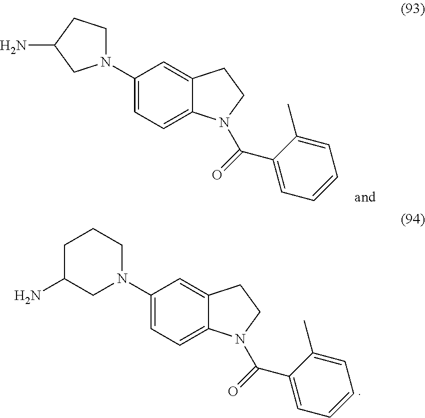 [SMILES: Cc1ccccc1C(=O)N1CCc2cc(N3CCC(N)C3)ccc21 ; Cc1ccccc1C(=O)N1CCc2cc(N3CCCC(N)C3)ccc21]

19. The compound of claim 11, wherein A and B are both phenyl and compound of formula (3) has the following structure: 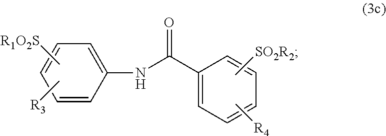
—SO₂R₁ and —SO₂R₂ can each be present or absent and, if present, R₁ and R₂ can each independently be substituted or unsubstituted heterocycloalkyl;
R₃ is selected from the group consisting of H, alkyl, O-alkyl and halogen;
R₄ is selected from the group consisting of H, alkyl, O-alkyl and halogen;
or an enantiomer, diastereomer, racemate or pharmaceutically acceptable salt, prodrug, or solvate thereof.

20. The compound of claim 18, wherein the compound of formula (3c) is selected from the group consisting of:

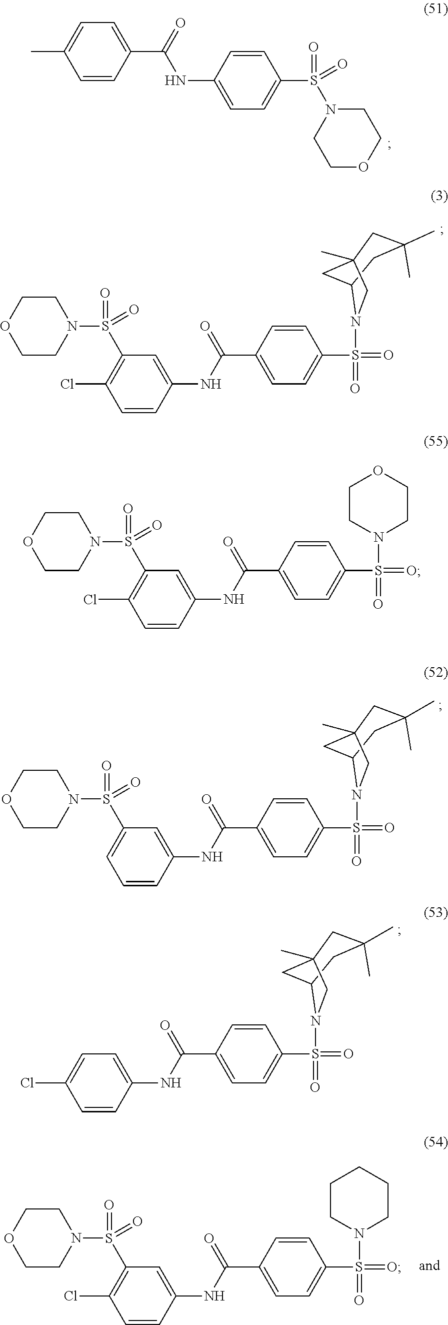

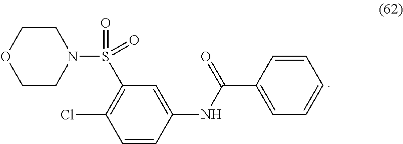

21. A method for treating or preventing a disease, disorder or condition associated with an Nrf2-regulated pathway, the method comprising administering at least one compound of formula (1), formula (2), or formula (3);
or an enantiomer, diastereomer, racemate or pharmaceutically acceptable salt, prodrug, or solvate thereof;
to a subject in an amount effective to decrease Nrf2 expression, thereby treating or preventing the disease, disorder, or condition.

22. The method of claim 21, wherein the disease, disorder or condition is associated with a disregulated Nrf2 activity.

23. The method of claim 21, wherein administering the at least one compound occurs in combination with another compound that affects an Nrf2-regulated gene to improve the efficacy of the another compound.

24. The method of claim 23, wherein the Nrf2-regulated gene is a gene that encodes for an efflux transporter or a metabolic protein.

25. The method of claim 21, wherein the at least one compound is administered before, during, or after administration of a chemotherapeutic drug and/or radiation therapy to the subject.

26. The method of claim 25, wherein administering the at least one compound enhances the efficacy of the chemotherapeutic drug and/or the radiation therapy.

27. The method of claim 25, wherein the chemotherapeutic drug is selected from the group consisting of a topoisomerase inhibitor, alkylating agent, antimetabolite, anthracycline, and plant alkoid.

28. The method of claim 27, wherein the chemotherapeutic drug is selected from the group consisting of etoposide, cisplatin, paclitaxel, gemcitabine, and carboplatin.

29. The method of claim 21, wherein the disease, disorder, or condition is cancer.

30. The method of claim 29, wherein the method suppresses tumor growth.

31. The method of claim 29, wherein the method inhibits or prevents the metastasis of a tumor.

32. The method of claim 21, wherein the at least one compound is administered by an administration route selected from the group consisting of oral, buccal, inhalation, sublingual, rectal, transdermal, vaginal, transmucosal, nasal or intestinal administration; parenteral delivery, including intramuscular, subcutaneous, intramedullary injections, intrathecal, direct intraventricular, intravenous, intra-articullar, intra-sternal, intra-synovial, intra-hepatic, intralesional, intracranial, intraperitoneal, intranasal, or intraocular injections.

33. The method of claim 21, wherein the method decreases Nrf2 transcription, Nrf2 translation, and/or Nrf2 biological activity.

34. The method of claim 21, wherein the at least one compound decreases an Nrf2 biological activity selected from the group consisting of binding of Nrf2 to an antioxidant-response element (ARE), nuclear accumulation of Nrf2, and the transcriptional induction of an Nrf2 target gene.

35. The method of claim 21, wherein the method attenuates the expression of at least one cytoprotective gene.

36. The method of claim 21, wherein the method downregulates the expression of at least one chemoresistant or radioresistant gene.

37. The method of claim 34, wherein the Nrf2 target gene is selected from the group consisting of MARCO, HO-1, NQO1, GCLm, GST α1, TrₓR₁, Pxr 1, GSR, G6PDH, GSS, GCLc, PGD, TKT, TALDO1, GST α3, GST p2, SOD2, SOD3, and GSR.

38. The method of claim 36, wherein the chemoresistant or radioresistant gene is GCLm or NQ01.

39. The method of claim 21, wherein the method attenuates at least one drug efflux pathway.
